(19) United States
(12) Patent Application Publication
(10) Pub. No.: US 2026/0250266 A1
(43) Pub. Date: Aug. 27, 2026

(54) HETEROCYCLIC PAN-KRAS INHIBITORS

(71) Applicant: Blueprint Medicines Corporation, Cambridge, MA (US)

(72) Inventors: Jason D. Brubaker, Cambridge, MA (US); Deborah G. Conrady, Arlington, MA (US); Brian J. Groendyke, Somerville, MA (US); Andrew Marc Haidle, Somerville, MA (US); Torsten Herbertz, Stow, MA (US); Richard Vargas, Bedford, MA (US)

(73) Assignee: Blueprint Medicines Corporation, Cambridge, MA (US)

(21) Appl. No.: 19/535,835

(22) Filed: Feb. 10, 2026

Related U.S. Application Data
(60) Provisional application No. 63/757,154, filed on Feb. 11, 2025.

(51) Int. Cl. C07D 401/14; C07D 471/04; C07D 487/04

(52) U.S. Cl. CPC C07D 401/14; C07D 471/04; C07D 487/04

(57) ABSTRACT

The present disclosure relates to compounds or pharmaceutically acceptable salts thereof, of Formula (I)

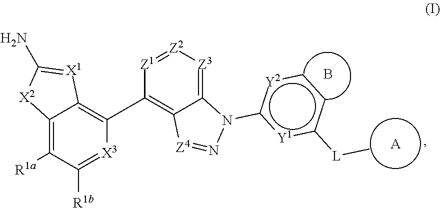

useful as pan-KRAS inhibitors, and methods of making same.

### BACKGROUND AND SUMMARY

This application claims priority to U.S. Application No. 63/757,154, filed Feb. 11, 2025, the entire content of which is incorporated by reference herein for all purposes.

The Kirsten rat sarcoma viral oncogene homolog (KRAS) gene is a small GTPase and a member of the rat sarcoma (RAS) family of genes that also includes Harvey rat sarcoma (HRAS) and neuroblastoma rat sarcoma (NRAS) viral oncogene homologs (Lee et al., Comprehensive pan-cancer genomic landscape of KRAS altered cancers and real-world outcomes in solid tumors, Npj Precision Oncology 2022, 6 (1); Liu et al., RAS-targeted cancer therapy: Advances in drugging specific mutations, MedComm. 2023, 4 (3)). The KRAS protein is part of the RAS/MAPK pathway, which signals the cell to proliferate or differentiate. To transmit these signals, the KRAS protein is activated by binding to GTP and is deactivated when the GTP is converted to GDP. Oncogenic KRAS is required for tumor cell maintenance in vitro and in vivo. KRAS alterations, particularly mutations, suppress KRAS intrinsic and GAP-induced GTPase activity, leading to an increased population of GTP-bound/active mutant KRAS protein in cancer cells (Liu et al., RAS-targeted cancer therapy: Advances in drugging specific mutations, MedComm. 2023, 4 (3)). This, in turn, leads to persistent activation of downstream effector pathways including RAF/MEK/ERK and PI3K/AKT/mTOR pathways.

In cancer cells associated with dependence on KRAS protein (e.g., cells with KRAS mutations, amplification, and overexpression), it is hoped that binders or inhibitors could deliver anti-cancer efficacy (e.g., inhibition of proliferation, survival, metastasis etc.). Genetic triple KRAS, HRAS, and NRAS knockout in adult mice causes death within a few weeks of induction (Drosten et al., Genetic analysis of Ras signaling pathways in cell proliferation, migration and survival, EMBO Journal 2010, 29 (6); Drosten et al., Ras signaling is essential for skin development, Oncogene 2014, 33 (22)). In agreement with the genetic triple RAS knockout data, RAS inhibitors in the clinic that target all three isoforms of RAS have high rates of adverse events, such as rash (cite https://www.revmed.com/wp-content/uploads/2024/10/2024_1019-RMC-6236-PDAC-ENA-POSTER-FINAL.pdf). However, genetic knockout of only KRAS in adult mice is well tolerated (PMID: 40825118). These preclinical and clinical results suggest that selectivity for KRAS over NRAS and HRAS is necessary to achieve a favorable risk: benefit ratio in the clinic.

23% of adult cancer samples (various tissue types) had KRAS alterations (e.g., mutation, over-expression, gene amplification) with KRAS activating mutations (e.g., the KRAS mutations G12D/G12V/G12C/G13D/G12R), which are associated with tumorigenesis as well as aggressive tumor growth, being among the most prevalent oncogenic driver mutations in human cancers (Lee et al., Comprehensive pan-cancer genomic landscape of KRAS altered cancers and real-world outcomes in solid tumors, Npj Precision Oncology 2022, 6 (1)). Moreover, KRAS alterations (e.g., mutation, over-expression, gene amplification) have also been described as a resistance mechanism against cancer drugs such as the EGFR antibodies cetuximab (Karapetis et al., K-ras Mutations and Benefit from Cetuximab in Advanced Colorectal Cancer, New England Journal of Medicine 2008, 359 (17)) and panitumumab (Amado et al., Wild-type KRAS is required for panitumumab efficacy in patients with metastatic colorectal cancer, Journal of Clinical Oncology 2008, 26 (10)) and the EGFR tyrosine kinase inhibitor osimertinib/AZD9291 (Xiu et al., Case Report: Outcome of Osimertinib Treatment in Lung Adenocarcinoma Patients With Acquired KRAS Mutations, Frontiers in Oncology 2021, 11). (Lee et al., Comprehensive pan-cancer genomic landscape of KRAS altered cancers and real-world outcomes in solid tumors, Npj Precision Oncology 2022, 6 (1)).

KRAS was long considered to be “undruggable” until 2013 when the Shokat lab identified covalent drugs targeting KRAS G12C(Ostrem et al., K-Ras (G12C) inhibitors allosterically control GTP affinity and effector interactions. Nature 2013, 503 (7477)). That discovery led to increased drug discovery research and eventually led to the first inhibitors targeting KRAS G12C: sotorasib and adagrasib (Hong et al., KRAS G12C Inhibition with Sotorasib in Advanced Solid Tumors, New England Journal of Medicine 2020, 383 (13); Jänne et al., Adagrasib in Non-Small-Cell Lung Cancer Harboring a KRAS G12C Mutation, New England Journal of Medicine 2022, 387 (2)). However, KRAS G12C specific inhibitors only target KRAS G12C. Similarly, KRAS G12D specific inhibitors only target KRAS G12D. Neither specific inhibitor type is effective against other KRAS mutations, since these inhibitors utilize specific interactions to the cysteine and aspartic acid residues of the KRAS mutants, respectively. For the KRAS G12C inhibitors, activity of the drug requires covalent binding of the drug to the thiol group on the cysteine (C) residue. For the KRAS G12D inhibitors, activity of the drugs requires noncovalent interactions between the drug and the aspartate (D) side chain. Since these previously described inhibitors require the presence of a specific mutant residue for binding, they are not able to target other KRAS mutant proteins, which remain an unmet need. Moreover, resistance to allele-specific KRAS inhibitors can involve the acquisition of additional mutations in KRAS which the allele-specific inhibitors cannot effectively target (see N. Engl. J. Med. 2021 Jun. 24; 384 (25): 2382-2393 (PMID: 34161704)), requiring the development of novel inhibitors that can still be efficacious in this setting. There are no FDA-approved targeted oncology treatment options for patients with types of KRAS alterations other than KRAS G12C as of today. Approaches to target other KRAS mutants could focus on binding to other regions of the KRAS protein, but since there is a high degree of similarity between KRAS and the other two RAS isoforms HRAS and NRAS, designing an inhibitor that interacts with residues specific to KRAS but not present in either HRAS or NRAS, and also not specific to a single KRAS point mutant, represents a significant technical challenge.

Therefore, there is an unmet medical need to develop new pan-KRAS inhibitors, in particular those that can inhibit all types of mutant KRAS, that present lower toxicity via exhibiting selectivity for KRAS over NRAS and HRAS and demonstrate sufficient efficacy for treating KRAS-driven cancers.

Disclosed herein are compounds of Formula (I), methods of making the same, and methods of treating a disease or disorder mediated by KRAS.

The present disclosure relates to a compound of Formula (I) or (I-a) or (I-b):

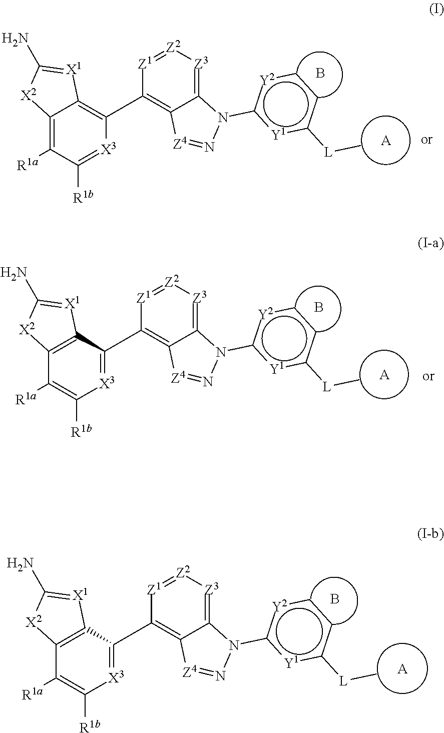

or a pharmaceutically acceptable salt thereof, wherein:
X¹ is N or C—CN;
X² is S or Se;
X³ is N or CR^{1c};
Z¹ is CR^{a} or N, Z² is CR^{b} or N; and Z³ is CR^{c} or N, provided that only one of Z¹, Z², and Z³ is N;
Z⁴ is CR^{d} or N;
Y¹ is CR^{e} or N;
Y² is CR^{f} or N;
wherein at least one of Y¹ and Y² is N;
L is selected from a bond, —(CR^{La}R^{Lb})ₙ—, —(CR^{La}R^{Lb})ₘ₁O(CR^{La}R^{Lb})ₘ₂—, and (CR^{La}R^{Lb})ₘ₁NR^{L}(CR^{La}R^{Lb})ₘ₂—;
R^{L} is H or C₁-C₄ alkyl;
R^{La} and R^{Lb} are each independently selected from H, D, and C₁-C₄ alkyl, wherein the alkyl is optionally substituted with 1 to 5 groups independently selected from D and halo;
n is selected from 1, 2, and 3;
m1 and m2 are each independently selected from 0, 1, 2, and 3, wherein m1+m2 is ≤4;
Ring A is a C₃-C₈ cycloalkyl, phenyl, 3 to 10 membered heterocyclyl ring, or 5 to 10 membered heteroaryl ring, wherein the cycloalkyl, phenyl, heterocyclyl, or heteroaryl ring are optionally substituted with 1 to 5 groups independently selected from D, halo, ═O, OR^{g}, NR^{g}R^{g}, C(O)NR^{g}R^{g}, —CN, C₁-C₄ alkyl, and C₂-C₄ alkenyl, and wherein the alkyl and alkenyl are optionally substituted with 1 to 5 groups independently selected from D, halo, ═O, OR^{g}, NR^{g}R^{g}C(O)NR^{g}R^{g}, CN, and 3 to 8 membered heterocyclyl ring optionally substituted with 1 to 5 groups independently selected from C₁-C₄ alkyl, wherein the heterocyclyl or heteroaryl ring comprises 1 or 2 heteroatoms selected from N, NR^{g}, O, and S;
Ring B is a C₃-C₁₂ cycloalkyl or 4 to 14 membered heterocyclyl, wherein the cycloalkyl or heterocyclyl are optionally substituted with 1 to 5 groups independently selected from D, halo, ═O, OR^{g}, NR^{g}R^{g}, C(O)NR^{g}R^{g}, —CN, C₁-C₄ alkyl, and C₂-C₄ alkenyl, wherein the alkyl and alkenyl are optionally substituted with 1 to 5 groups independently selected from D, halo, ═O, OR^{g}, NR^{g}R^{g}, C(O)NR^{g}R^{g}, and CN, wherein the heterocyclyl comprises 1 or 2 heteroatoms selected from N, NR^{g}, O, and S; or
R^{1a}, R^{1b}, and R^{1c} are each independently selected from H, D, halo, and C₁-C₄ alkyl, wherein the alkyl is optionally substituted with 1 to 3 groups independently selected from D and halo;
R^{a}, R^{b}, R^{c} and R^{d} are each independently selected from H, D, halo, CN, OR^{g}, N(R^{g})₂, C₁-C₄ alkyl, C₃-C₆ cycloalkyl, 5 to 6 membered heterocyclyl, and 5 or 6 membered heteroaryl, wherein the alkyl is optionally substituted with 1 to 5 groups independently selected from D, halo, ═O, OR^{g}, N(R^{g})₂C(O)NR^{g}R^{g}, 5 to 6 membered heterocyclyl and 5 to 6 membered heteroaryl, and wherein the heteroaryl and heterocyclyl comprises 1-3 heteroatoms selected from NR^{g}, O, and S and are optionally substituted with 1 to 5 groups independently selected from D, halo, ═O, C₁-C₄ alkyl, and C₁-C₄ alkoxy, wherein the alkyl and alkoxy are optionally substituted with 1 to 5 groups independently selected from D, halo, and OR^{g}, and the cycloalkyl is optionally substituted with 1 to 5 groups independently selected from D, halo, ═O, C₁-C₄ alkyl, and C₁-C₄ alkoxy, wherein the alkyl and alkoxy are optionally substituted with 1 to 5 groups independently selected from D, halo, and OR^{g}; or
R^{a} and R^{b}, together with the carbon atoms to which they are attached, form a cyclopentyl, cyclohexyl, or a 5 to 6 membered heterocyclyl, wherein the cyclopentyl, cyclohexyl, or 5 to 6 membered heterocyclyl are optionally substituted with 1 to 3 groups independently selected from D, halo, C₁-C₄ alkyl, and C₁-C₄ alkoxy, wherein the alkyl and alkoxy are optionally substituted with 1 to 5 groups independently selected from D, halo, and OR^{g}, and the heterocyclyl comprises 1 or 2 heteroatoms selected from N, NR^{g}, O, and S; or
R^{b} and R^{c}, together with the carbon atoms to which they are attached, form a cyclopentyl, cyclohexyl, or a 5 to 6 membered heterocyclyl, wherein the cyclopentyl, cyclohexyl, or 5 to 6 membered heterocyclyl are optionally substituted with 1 to 3 groups independently selected from D, halo, C₁-C₄ alkyl, and C₁-C₄ alkoxy, wherein the alkyl and alkoxy are optionally substituted with 1 to 5 groups independently selected from D, halo, and OR^{g}, and the heterocyclyl comprises 1 or 2 heteroatoms selected from N, NR^{g}, O, and S;
R^{e} and R^{f} are each selected from H, halo, and C₁-C₄ alkyl, wherein the alkyl is optionally substituted with 1 to 5 groups independently selected from D and halo; and
R^{g} is each independently selected from H and C₁-C₄ alkyl, wherein the alkyl is optionally substituted with 1 to 5 groups independently selected from D and halo.

The present disclosure also relates to the compounds of Table 1, or pharmaceutically acceptable salts thereof.

The present disclosure further relates to a method of treating a disease or disorder mediated by KRAS, comprising providing to a subject in need thereof a compound of Formula (I) or (I-a) or (I-b):

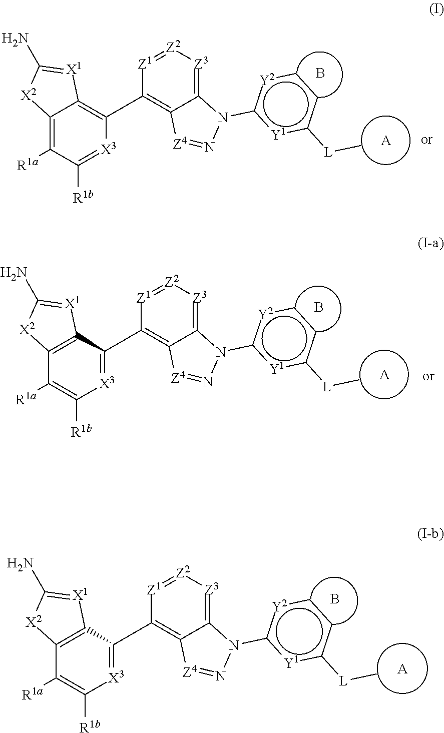

or a pharmaceutically acceptable salt thereof.

### DETAILED DESCRIPTION

### Definitions

Unless otherwise defined, all terms of art, notations and other scientific terminology used herein are intended to have the meanings commonly understood by those of skill in the art to which this disclosure pertains. In some cases, terms with commonly understood meanings are defined herein for clarity and/or for ready reference.

Unless otherwise indicated, the following terms have the following meanings:

The term “alkyl” used alone or as part of a larger moiety, such as “alkoxy”, “alkenyl”, “alkynyl”, and the like, refers to an aliphatic straight-chain or branched hydrocarbon moiety. Unless otherwise specified, an alkyl has 1, 2, 3, 4, 5, or 6 carbon atoms (i.e., C₁-C₆ alkyl) or 1, 2, 3, or 4 carbon atoms (i.e., C₁-C₄ alkyl). Examples of an alkyl include, but are not limited to, methyl, ethyl, propyl (or n-propyl or 1-propyl or prop-1-yl), isopropyl (or i-propyl or 2-propyl or prop-2-yl), butyl (or n-butyl or 1-butyl or but-1-yl), isobutyl (or i-butyl or 2-methylpropyl), sec-butyl (s-butyl or but-2-yl or 2-methylpropyl), tert-butyl (or t-butyl), pentyl (or n-pentyl or pent-1-yl), isopentyl (or i-pentyl or 3-methylbut-1-yl), sec-pentyl (or s-pentyl or pent-2-yl or 1-methylbut-1-yl), 2-methylbutan-2-yl (or 1,1-dimethylpropyl or tert-pentyl or t-pentyl), 2,2-dimethylpropyl (or 2,2,2-trimethyleth-1-yl), 3-pentyl (or pent-3-yl or 1-ethylpropyl), 3-methylbut-2-yl (or 1,2-dimethylpropyl or sec-isopentyl), 2-methylbut-2-yl, hexyl (or n-hexyl), and the like.

The term “alkenyl” refers to an alkyl in which one or more carbon/carbon single bonds is replaced by a double bond. Unless otherwise specified, an alkenyl has 2, 3, 4, 5, or 6 carbon atoms (i.e., C₂-C₆ alkenyl) or 2, 3, or 4 carbon atoms (i.e., C₂-C₄ alkenyl). Examples of an alkenyl include, but are not limited to, ethenyl, 1-propen-1-yl, 1-propen-2-yl, 2-propen-1-yl, 1-buten-1-yl, 1-buten-2-yl, 3-buten-1-yl, 3-buten-2-yl, 2-buten-1-yl, 2-buten-2-yl, 2-methyl-1-propen-1-yl, 2-methyl-2-propen-1-yl, 1,3-butadien-1-yl, 1,3-butadien-2-yl, and the like.

The term “alkoxy” refers to an alkyl attached through an oxygen linking atom, represented by —O-alkyl. Unless otherwise specified, an alkoxy has 1, 2, 3, 4, 5, or 6 carbon atoms (i.e., C₁-C₆ alkoxy) or 1, 2, 3, or 4 carbon atoms (i.e., C₁-C₄ alkoxy). Examples of an alkoxy include, but are not limited to, methoxy, ethoxy, propoxy (or n-propoxy or 1-propoxy or prop-1-yloxy), isopropoxy (or i-propoxy or 2-propoxy or prop-2-yloxy), butoxy (or n-butoxy or 1-butoxy or but-1-yloxy), isobutoxy (or i-butoxy or 2-methylpropoxy), sec-butoxy (s-butoxy or but-2-yloxy or 2-methylpropoxy), tert-butoxy (or t-butoxy), pentoxy (or n-pentoxy or pent-1-yloxy), isopentoxy (or i-pentoxy or 3-methylbut-1-yloxy), sec-pentoxy (or s-pentoxy or pent-2-yloxy or 1-methylbut-1-yloxy), 2-methylbutan-2-yloxy (or 1,1-dimethylpropoxy or tert-pentoxy or t-pentoxy), 2,2-dimethylpropoxy (or 2,2,2-trimethyleth-1-yloxy), 3-pentoxy (or pent-3-yloxy or 1-ethylpropyloxy), 3-methylbut-2-yloxy (or 1,2-dimethylpropyloxy or sec-isopentoxy), 2-methylbut-2-yloxy, hexoxy (or n-hexoxy), and the like.

The term “aryl” refers to a monocyclic or bicyclic aromatic hydrocarbon ring system. Unless otherwise specified, an aryl has 6, 7, 8, 9, 10, 11, 12, 13, or 14 carbon atoms (i.e., C₆-C₁₄ aryl); 6, 7, 8, 9, 10, 11, or 12 carbon atoms (i.e., C₆-C₁₂ aryl); or 6 carbon atoms (i.e., C₆ aryl or a phenyl ring). Examples of an aryl include, but are not limited to, phenyl, cyclooctatetraenyl, and naphthyl, and the like.

The term “cycloalkyl” refers to a monocyclic or bicyclic hydrocarbon saturated ring system, wherein at least one ring is non-aromatic. Examples of a cycloalkyl include, but are not limited to, cyclopropyl, cyclobutyl, cyclopentyl, cyclohexyl, cycloheptyl, cyclooctyl, and the like.

Unless otherwise specified, a cycloalkyl has 3, 4, 5, 6, 7, 8, 9, 10, 11, or 12 carbon atoms (i.e., C₃-C₁₂ cycloalkyl); 3, 4, 5, 6, 7, 8, 9, or 10 carbon atoms (i.e., C₃-C₁₀ cycloalkyl); 3, 4, 5, 6, 7, or 8 carbon atoms (i.e., C₃-C₈ cycloalkyl); 5, 6, 7, 8, 9, or 10 carbon atoms (i.e., C₅-C₁₀ cycloalkyl); or 3, 4, 5, or 6 carbon atoms (i.e., C₃-C₆ cycloalkyl).

A bicyclic cycloalkyl may include a fused, bridged, or spiro ring:

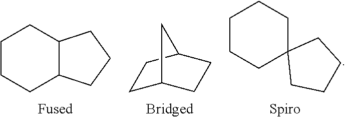

A fused cycloalkyl includes two rings sharing two adjacent carbon atoms, wherein at least one of the rings is non-aromatic. Examples of a fused cycloalkyl include, but are not limited to, decahydronaphthalene, indanyl, indenyl, tetrahydronaphthyl, bicyclo[1.1.0]butyl, bicyclo[2.1.0]pentyl, bicyclo[3.1.0]hexyl, bicyclo[3.2.0]heptyl, bicyclo[4.1.0]heptyl, bicyclo[4.2.0]octyl, and the like.

A bridged cycloalkyl includes two rings sharing two non-adjacent carbon atoms, wherein at least one of the rings is non-aromatic. Examples of a bridged cycloalkyl include, but are not limited to, bicyclo[2.1.1]hexyl, bicyclo[2.2.1]heptyl, bicyclo[3.1.1]heptyl, bicyclo[3.2.1]octyl, bicyclo[2.2.2]octyl, bicyclo[4.3.1]decane, and the like.

A spiro cycloalkyl includes two rings sharing one carbon atom, wherein at least one of the rings is non-aromatic. Examples of a spiro cycloalkyl ring include, but are not limited to, spiro[2.2]pentane, spiro[2.3]hexane, spiro[2.5]octane, spiro[2.6]nonane, spiro[3.5]nonane, spiro[4.4]nonane, spiro[2.6]nonane, spiro[4.5]decane, spiro[3.6]decane, spiro[5.5]undecane, and the like.

The term “heteroaryl” refers to a monocyclic or bicyclic aromatic ring system having ring carbon atoms and 1, 2, 3, or 4 ring heteroatoms, wherein each heteroatom is independently selected from nitrogen, substituted nitrogen, oxygen, and sulfur. Heteroaryl ring systems may include heteroatoms in one or more rings in the ring system. Unless otherwise specified, a heteroaryl has 5, 6, 7, 8, 9, 10, 11, 12, 13, or 14 ring atoms (i.e., 5- to 14-membered heteroaryl, wherein the term “membered” refers to the non-hydrogen ring atoms within the ring system); 5, 6, 7, 8, 9, 10, 11, or 12 ring atoms (i.e., 5- to 12-membered heteroaryl); 5, 6, 7, 8, 9, or 10 ring atoms (i.e., 5- to 10-membered heteroaryl); 5, 6, 7, or 8 ring atoms (i.e., 5- to 8-membered heteroaryl); or 5 or 6 ring atoms (i.e., 5- to 6-membered heteroaryl), wherein each heteroaryl has 1, 2, 3, or 4 ring heteroatoms; 1, 2, or 3 ring heteroatoms; or 1 or 2 ring heteroatoms, wherein each heteroatom is independently selected from nitrogen, substituted nitrogen, oxygen, and sulfur. Examples of a heteroaryl include, but are not limited to, furanyl, pyrrolyl, thiophenyl, imidazolyl, oxazolyl, pyrazolyl, thiazolyl, isothiazolyl, isoxazolyl, oxadiazolyl, thiadiazolyl, triazolyl, tetrazolyl, pyridinyl, pyrazinyl, pyridazinyl, pyrimidinyl, triazinyl, and the like.

A heteroaryl may include fused rings (two rings sharing two adjacent atoms) wherein one heteroaryl ring is fused with one or more heteroaryl or aryl. Examples of a fused heteroaryl include, but are not limited to, purinyl, benzo[b]thiophen-2-yl, benzo[c]thiophen-1-yl, indolizinyl, cinnolinyl, quinazolinyl, pteridinyl, and the like.

The term “heterocyclyl” refers to a monocyclic or bicyclic non-aromatic ring system having ring carbon atoms and 1, 2, 3, or 4 ring heteroatoms, wherein each heteroatom is independently selected from nitrogen, substituted nitrogen, oxygen, and sulfur. Heterocyclyl ring systems may include heteroatoms in one or more rings in the ring system. A heterocyclyl may be a saturated ring system (i.e., heterocycloalkyl) or may include one or more double bonds in the ring system (i.e., heterocycloalkenyl). Examples of a heterocyclyl include, but are not limited to, azetidinyl, oxetanyl, thietanyl, pyrrolidinyl, tetrahydrofuranyl, dioxolanyl, dithiolanyl, isoxazolinyl, oxazolinyl, pyrazolidinyl, thiazolidinyl, dithiazolyl, piperidinyl, tetrahydropyranyl, tetrahydropyridinyl, thianyl, dioxanyl, morpholinyl, piperazinyl, thiomorpholinyl, azepanyl, oxepanyl, thiepanyl, and oxazepanyl. Examples of a heterocycloalkyl include, but are not limited to, azetidinyl, oxetanyl, pyrrolidinyl, tetrahydrofuranyl, dioxolanyl, pyrazolidinyl, piperidinyl, tetrahydropyranyl, morpholinyl, piperazinyl, azepanyl, and oxazepanyl, and the like.

Unless otherwise specified, a heterocyclyl has 3, 4, 5, 6, 7, 8, 9, 10, 11, or 12 ring atoms (i.e., 3- to 12-membered heterocyclyl, wherein the term “membered” refers to the non-hydrogen ring atoms within the ring system); 4, 5, 6, 7, 8, 9, or 10 ring atoms (i.e., 4- to 10-membered heterocyclyl); 3, 4, 5, 6, 7, or 8 ring atoms (i.e., 3- to 8-membered heterocyclyl); 5, 6, 7, or 8 ring atoms (i.e., 5- to 8-membered heterocyclyl), or 4, 5, or 6 ring atoms (i.e., 4- to 6-membered heterocyclyl), wherein each heterocyclyl has 1, 2, 3, or 4 ring heteroatoms; 1, 2, or 3 ring heteroatoms; or 1 or 2 ring heteroatoms, wherein each heteroatom is independently selected from nitrogen, substituted nitrogen, oxygen, and sulfur.

A fused heterocyclyl includes two rings sharing two adjacent atoms, wherein at least one of the rings is non-aromatic. Examples of a fused heterocyclyl include, but are not limited to, hexahydropyrrolo[1,2-a]pyrazin-2(1H)-yl, hexahydro-1H-pyrrolizinyl, and the like.

A bridged heterocyclyl includes two rings sharing two non-adjacent atoms, wherein at least one of the rings is non-aromatic. Examples of a bridged heterocyclyl include, but are not limited to, 8-oxabicyclo[3.2.1]octanyl, 7-azabicyclo[2.2.1]heptanyl, 3,9-diazabicyclo[4.2.1]nonan-9-yl, 2-oxabicyclo[2.2.1]heptanyl, 6-oxabicyclo[3.1.1]heptanyl, 7-oxabicyclo[2.2.1]heptanyl, and the like.

A spiro heterocyclyl includes two rings sharing one atom, wherein at least one of the rings is non-aromatic. Examples of a spiro heterocyclyl include, but are not limited to, 5-azaspiro[2.4]heptanyl, 2,7-dioxaspiro[4.5]decanyl, and the like.

The aryl, cycloalkyl, heteroaryl, and heterocyclyl each may be attached through any ring atom unless such attachment would violate valence requirements.

The term “halo” refers to a halogen atom and includes chloro, fluoro, bromo and iodo.

The term “oxo” refers to a double bonded oxygen (═O).

The term “substituted” refers to when one or more hydrogens on the designated atom is replaced with a selection from the indicated substituents, provided that the designated atom's normal valency under the existing circumstances is not exceeded, and that the substitution results in a stable compound. The substitutions may take place independently of one another, in the form of mono- or polysubstitutions in each case, on all the hydrogen-carrying atoms. Substituents do not count towards the number of atoms. Combinations of substituents are permissible only if such combinations result in stable compounds. Polymers or similar indefinite structures arrived at by defining substituents with further substituents appended ad infinitum (e.g., a substituted aryl having a substituted alkyl which is itself substituted with a substituted aryl, which is further substituted by a substituted alkyl, etc.) are not intended for inclusion herein. Similarly, the above definitions are not intended to include impermissible substitution patterns (e.g., methyl substituted with 5 fluoro moieties or heteroaryl having two adjacent oxygen ring atoms). Such impermissible substitution patterns are well known to the skilled artisan.

The expression “optionally substituted” refers to (1) unsubstituted or (2) substituted.

The expression “compounds of the disclosure” refers to compounds of Formula (I), (I-a), (I-b), (II), (II-a), (II-b), (III), (III-a), (III-b), (IVa), (IVa-a), (IVa-b), (IVb), (IVb-a), (IVb-b), (IVc), (IVc-a), (IVc-b), (IVd), (IVd-a), (IVd-b), (IVe), (IVe-a), (IVe-b), (IVf), (IVf-a), (IVf-b), (IVg), (IVg-a), (IVg-b), (IVh), (IVh-a), (IVh-b), (IVi), (IVi-a), (IVi-b), (IVj), (IVj-a), (IVj-b), (IVk), (IVk-a), (IVk-b), (IVl), (IVl-a), (IVl-b), (IVm), (IVm-a), (IVm-b), (IVn), (IVn-a), (IVn-b), (Va), (Va-a), (Va-b), (Vb), (Vb-a), (Vb-b), (Vc), (Vc-a), (Vc-b), (Vd), (Vd-a), (Vd-b), (Ve), (Ve-a), (Ve-b), (Vf), (Vf-a), (Vf-b), (Vg), (Vg-a), (Vg-b), (Vh), (Vh-a), (Vh-b), (VIa), (VIa-a), (VIa-b), (VIb), (VIb-a), (VIb-b), (VIc), (VIc-a), (VIc-b), (VId), (VId-a), (VId-b), (VIe), (VIe-a), (VIe-b), (VIf), (VIf-a), (VIf-b), (VIg), (VIg-a), (VIg-b), (VIh), (VIh-a), (VIh-b), (VIi), (VIi-a), (VIi-b), (VIj), (VIj-a), (VIj-b), (VIk), (VIk-a), (VIk-b), (VIl), (VIl-a), (VIl-b), (VIm), (VIm-a), (VIm-b), (VIn), (VIn-a), (VIn-b), (VIo), (VIo-a), (VIo-b), (VIp), (VIp-a), (VIp-b), (VIq), (VIq-a), (VIq-b), (VIr), (VIr-a), (VIr-b), (VIs), (VIs-a), (VIs-b), (VIt), (VIt-a), (VIt-b), (VIu), (VIu-a), (VIu-b), (VIv), (VIv-a), (VIv-b), (VIw), (VIw-a), (VIw-b), (VIx), (VIx-a), (VIx-b), (VIy), (VIy-a), (VIy-b), (VIz), (VIz-a), (VIz-b), (VIaa), (VIaa-a), (VIaa-b), (VIab), (VIab-a), (VIab-b), (VIac), (VIac-a), (VIac-b), (VIad), (VIad-a), (VIad-b), (VIae), (VIae-a), (VIae-b), (VIaf), (VIaf-a), (VIaf-b), (VIag), (VIag-a), (VIag-b), (VIah), (VIah-a), (VIah-b), (VIai), (VIai-a), (VIai-b), (VIaj), (VIaj-a), (VIaj-b), (VIak), (VIak-a), (VIak-b), (VIal), (VIal-a), (VIal-b), (VIam), (VIam-a), (VIam-b), (VIan), (VIan-a), (VIan-b), (VIao), (VIao-a), or (VIao-b).

The expression “stable compound” refers to a compound of the disclosure which can be prepared and isolated and whose structure and properties remain or can be caused to remain essentially unchanged for a period of time sufficient to allow use of the compound of the disclosure for the purposes described herein (e.g., therapeutic administration to a patient).

The term “inhibitor” refers to a compound that has a biological effect to inhibit or significantly reduce or down-regulate the biological activity of a gene and/or a protein. Consequently, the expression a “KRAS inhibitor” refers to a compound that has a biological effect to inhibit or significantly reduce or down-regulate the biological activity of KRAS, for example, a mutant KRAS or wild type KRAS. The expression “pan-KRAS inhibitor” refers to a “KRAS inhibitor” that is active against KRAS across multiple KRAS mutants and/or wild type KRAS. In one embodiment, the compounds of the disclosure are KRAS inhibitors. In one embodiment, the compounds of the disclosure are pan-KRAS inhibitors. In some embodiments, the compounds of this disclosure are pan-KRAS inhibitors active against one of more KRAS mutations selected from KRAS^{G12C}, KRAS^{G12v}, KRAS^{G12D}, KRAS^{G12A}, KRAS^{G12S}, and KRAS^{G13D}. In some embodiments, the KRAS inhibitors and pan-KRAS inhibitors of the present disclosure do not show activity against NRAS and/or HRAS. In some embodiments, the compounds of the disclosure are selective for KRAS over NRAS. In some embodiments, the compounds of the disclosure are selective for KRAS over HRAS. In some embodiments, the compounds of the disclosure are selective for KRAS over NRAS and HRAS. In some embodiments, the compounds of the disclosure show at least about 5, 10, 50, 100, 200, 300, 400, 500, or 1000 selectivity for KRAS versus HRAS. In some embodiments, the compounds of the disclosure show at least about 5, 10, 50, 100, 200, 300, 400, 500, or 1000 selectivity for KRAS versus NRAS. In some embodiments, the compounds of the disclosure show at least about 5, 10, 50, 100, 200, 300, 400, 500, or 1000 selectivity for KRAS versus HRAS and NRAS. The ability to selectively target KRAS with a compound of the disclosure may provide advantages in terms of less off-target activity, and an increased probability of tolerability and therefore clinical success in comparison with a non-selective compound.

The expression “pharmaceutically acceptable excipient” or the term “excipient” refer to a broad range of ingredients that may be combined with the compounds of the disclosure in a pharmaceutical composition.

The expression “pharmaceutically acceptable salt” refers to a pharmaceutical salt that is, within the scope of sound medical judgment, suitable for use in a patient without undue toxicity, irritation, and allergic response, and is commensurate with a reasonable benefit/risk ratio.

The term “salt” refers to a salt that may be formed by mixing a compound of the disclosure as a free base or a free acid with an acid or a base, respectively.

The terms “administer”, “administering”, “administration”, and the like, refer to methods that may be used to enable delivery of a compound of the disclosure, e.g., in a pharmaceutical composition, to the desired site of biological action.

The term “patient” refers to a human in need of medical treatment.

The terms “treating” or “treatment” refer to obtaining a desired biological effect, pharmacological effect, or physiological effect, or a combination thereof in a patient. The effect may be therapeutic, which includes achieving, partially or substantially, one or more of the following results: reducing the extent of the disease; ameliorating or improving a clinical symptom or indicator associated with the disease; stabilizing the disease; delaying, inhibiting or decreasing the likelihood of the progression of the disease; decreasing the likelihood of recurrence of the disease; or prolonging the survival of a patient with the disease.

The present disclosure relates to compounds of Formula (I) or (I-a) or (I-b):

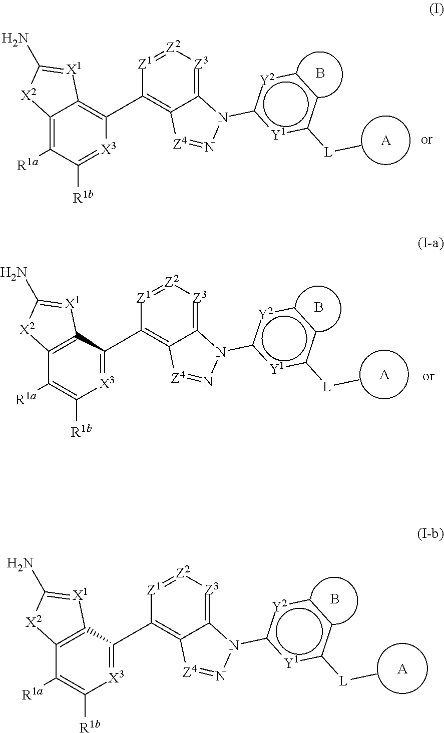

(1) or a pharmaceutically acceptable salt thereof, wherein:
X¹ is N or C—CN;
X² is S or Se;
X³ is N or CR^{1c};
Z¹ is CR^{a} or N, Z² is CR^{b} or N; and Z³ is CR^{c} or N, provided that only one of Z¹, Z², and Z³ is N;
Z⁴ is CR^{d} or N;
Y¹ is CR^{e} or N;
Y² is CR^{f} or N;
wherein at least one of Y¹ and Y² is N;
L is selected from a bond, —(CR^{La}R^{Lb})ₙ—, —(CR^{La}R^{Lb})ₘ₁O(CR^{La}R^{Lb})ₘ₂—, and (CR^{La}R^{Lb})ₘ₁NR^{L}(CR^{La}R^{Lb})ₘ₂—;
R^{L} is H or C₁-C₄ alkyl;
R^{La} and R^{Lb} are each independently selected from H, D, and C₁-C₄ alkyl, wherein the alkyl is optionally substituted with 1 to 5 groups independently selected from D and halo;
n is selected from 1, 2, and 3;
m1 and m2 are each independently selected from 0, 1, 2, and 3, wherein m1+m2 is ≤4;
Ring A is a C₃-C₈ cycloalkyl, phenyl, 3 to 10 membered heterocyclyl ring, or 5 to 10 membered heteroaryl ring, wherein the cycloalkyl, phenyl, heterocyclyl, or heteroaryl ring are optionally substituted with 1 to 5 groups independently selected from D, halo, ═O, OR^{g}, NR^{g}R^{g}, C(O)NR^{g}R^{g}, —CN, C₁-C₄ alkyl, and C₂-C₄ alkenyl, and wherein the alkyl and alkenyl are optionally substituted with 1 to 5 groups independently selected from D, halo, ═O, OR^{g}, NR^{g}R^{g}, C(O)NR^{g}R^{g}, CN, and 3 to 8 membered heterocyclyl ring optionally substituted with 1 to 5 groups independently selected from C₁-C₄ alkyl, wherein the heterocyclyl or heteroaryl ring comprises 1 or 2 heteroatoms selected from N, NR^{g}, O, and S;
Ring B is a C₃-C₁₂ cycloalkyl or 4 to 14 membered heterocyclyl, wherein the cycloalkyl or heterocyclyl are optionally substituted with 1 to 5 groups independently selected from D, halo, ═O, OR^{g}, NR^{g}R^{g}, C(O)NR^{g}R^{g}, —CN, C₁-C₄ alkyl, and C₂-C₄ alkenyl, wherein the alkyl and alkenyl are optionally substituted with 1 to 5 groups independently selected from D, halo, ═O, OR^{g}, NR^{g}R^{g}, C(O)NR^{g}R^{g}, and CN, wherein the heterocyclyl comprises 1 or 2 heteroatoms selected from N, NR^{g}, O, and S; or
R^{1a}, R^{1b}, and R^{1c} are each independently selected from H, D, halo, and C₁-C₄ alkyl, wherein the alkyl is optionally substituted with 1 to 3 groups independently selected from D and halo;
R^{a}, R^{b}, R^{c} and R^{d} are each independently selected from H, D, halo, CN, OR^{g}, N(R^{g})₂, C₁-C₄ alkyl, C₃-C₆ cycloalkyl, 5 to 6 membered heterocyclyl, and 5 or 6 membered heteroaryl, wherein the alkyl is optionally substituted with 1 to 5 groups independently selected from D, halo, ═O, OR^{g}, N(R^{g})₂C(O)NR^{g}R^{g}, 5 to 6 membered heterocyclyl and 5 to 6 membered heteroaryl, and wherein the heteroaryl and heterocyclyl comprises 1-3 heteroatoms selected from NR^{g}, O, and S and are optionally substituted with 1 to 5 groups independently selected from D, halo, ═O, C₁-C₄ alkyl, and C₁-C₄ alkoxy, wherein the alkyl and alkoxy are optionally substituted with 1 to 5 groups independently selected from D, halo, and ORS, and the cycloalkyl is optionally substituted with 1 to 5 groups independently selected from D, halo, O, C₁-C₄ alkyl, and C₁-C₄ alkoxy, wherein the alkyl and alkoxy are optionally substituted with 1 to 5 groups independently selected from D, halo, and OR^{g}; or
R^{a} and R^{b}, together with the carbon atoms to which they are attached, form a cyclopentyl, cyclohexyl, or a 5 to 6 membered heterocyclyl, wherein the cyclopentyl, cyclohexyl, or 5 to 6 membered heterocyclyl are optionally substituted with 1 to 3 groups independently selected from D, halo, C₁-C₄ alkyl, and C₁-C₄ alkoxy, wherein the alkyl and alkoxy are optionally substituted with 1 to 5 groups independently selected from D, halo, and OR^{g}, and the heterocyclyl comprises 1 or 2 heteroatoms selected from N, NR^{g}, O, and S; or
R^{b} and R^{c}, together with the carbon atoms to which they are attached, form a cyclopentyl, cyclohexyl, or a 5 to 6 membered heterocyclyl, wherein the cyclopentyl, cyclohexyl, or 5 to 6 membered heterocyclyl are optionally substituted with 1 to 3 groups independently selected from D, halo, C₁-C₄ alkyl, and C₁-C₄ alkoxy, wherein the alkyl and alkoxy are optionally substituted with 1 to 5 groups independently selected from D, halo, and ORS, and the heterocyclyl comprises 1 or 2 heteroatoms selected from N, NR^{g}, O, and S;
R^{e} and R^{f} are each selected from H, halo, and C₁-C₄ alkyl, wherein the alkyl is optionally substituted with 1 to 5 groups independently selected from D and halo; and
R^{g} is each independently selected from H and C₁-C₄ alkyl, wherein the alkyl is optionally substituted with 1 to 5 groups independently selected from D and halo.

In addition to embodiment (1) in the preceding paragraphs, the compounds of Formula (I) or (I-a) or (I-b), or pharmaceutically acceptable salts thereof, include those in which:

(2) the compound is of Formula (II) or (II-a) or (II-b):

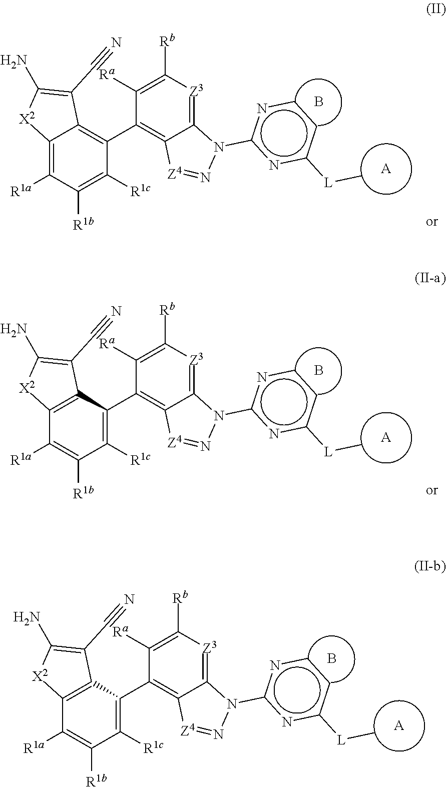

or a pharmaceutically acceptable salt thereof, wherein:
X² is S or Se;
Z³ is CR^{c} or N;
Z⁴ is CR^{d} or N;
L is selected from a bond and O(CR^{La}R^{Lb})ₘ₂—;
R^{La} and R^{Lb} are each independently selected from H, D, and C₁-C₄ alkyl, wherein the alkyl is optionally substituted with 1 to 5 groups independently selected from D and halo;
m2 is independently selected from 0, 1, and 2;
Ring A is a C₃-C₈ cycloalkyl or 3 to 10 membered heterocyclyl ring, wherein the cycloalkyl or heterocyclyl are optionally substituted with 1 to 5 groups independently selected from D, halo, ═O, OR^{g}, NR^{g}R^{g}, and C₁-C₄ alkyl, and wherein the alkyl is optionally substituted with 1 to 5 groups independently selected from D and halo, wherein the heterocyclyl ring comprises 1 or 2 heteroatoms selected from N, NR^{g}, O, and S;
Ring B is a C₃-C₈ cycloalkyl or 5 to 12 membered heterocyclyl, wherein the cycloalkyl or heterocyclyl are optionally substituted with 1 to 5 groups independently selected from D, halo, ═O, OR^{g}, and C₁-C₄ alkyl, wherein the alkyl is optionally substituted with 1 to 5 groups independently selected from D, halo, ═O, OR^{g}, and NR^{g}R^{g}, wherein the heterocyclyl comprises 1 or 2 heteroatoms selected from N, NR^{g}, O, and S; or
R^{1a}, R^{1b}, and R^{1c} are each independently selected from H, D, halo, and C₁-C₄ alkyl;
R^{a}, R^{b}, R^{c} and R^{d} are each independently selected from H, D, halo, OR^{g}, N(R^{g})₂, C₁-C₄ alkyl, and C₃-C₆ cycloalkyl, wherein the alkyl is optionally substituted with 1 to 5 groups independently selected from D, halo, and OR^{g}, and wherein the cycloalkyl is optionally substituted with 1 to 5 groups independently selected from halo, ═O, C₁-C₄ alkyl, and C₁-C₄ alkoxy, wherein the alkyl and alkoxy are optionally substituted with 1 to 5 groups independently selected from halo and OR^{g}; or
R^{a} and R^{b}, together with the carbon atoms to which they are attached, form a cyclopentyl, cyclohexyl, or a 5 to 6 membered heterocyclyl, wherein the heterocyclyl comprises 1 or 2 oxygen atoms; or
R^{b} and R^{c}, together with the carbon atoms to which they are attached, form a cyclopentyl, cyclohexyl, or a 5 to 6 membered heterocyclyl, wherein the heterocyclyl comprises 1 or 2 oxygen atoms; and
R^{g} is each independently selected from H and C₁-C₄ alkyl, wherein the alkyl is optionally substituted with 1 to 5 groups independently selected from D and halo, and the remainder of the variables are as described in embodiment (1).

In addition to embodiments (1) and (2) in the preceding paragraphs, the compounds of Formula (I), (I-a), (I-b), (II), (II-a), or (II-b) or pharmaceutically acceptable salts thereof, include those in which:

(3) the compound is of Formula (III) or (III-a) or (III-b):

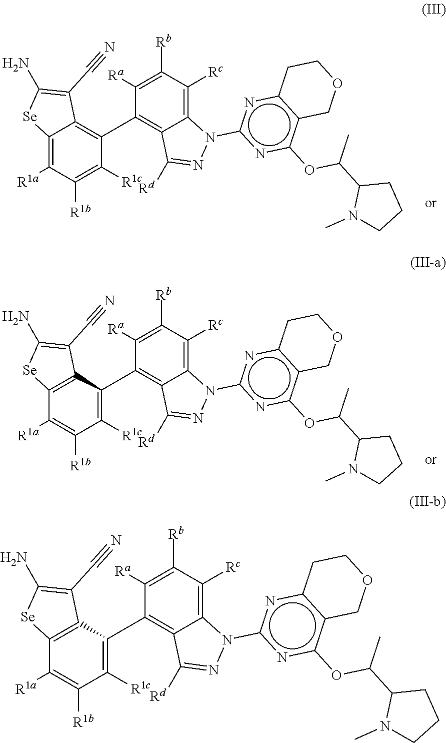

or a pharmaceutically acceptable salt thereof, wherein:
R^{1a}, R^{1b}, and R^{1c} are each independently selected from H, halo, and C₁-C₄ alkyl;
R^{a}, R^{b}, R^{c} and R^{d} are each independently selected from H, D, halo, OR^{g}, N(R^{g})₂, C₁-C₄ alkyl, and C₃-C₆ cycloalkyl, wherein the alkyl is optionally substituted with 1 to 5 groups independently selected from D, halo, and ORS, and wherein the cycloalkyl is optionally substituted with 1 to 5 groups independently selected from halo, ═O, C₁-C₄ alkyl, and C₁-C₄ alkoxy, wherein the alkyl and alkoxy are optionally substituted with 1 to 5 groups independently selected from halo and OR^{g}; or
R^{g} is each independently selected from H and C₁-C₄ alkyl, wherein the alkyl is optionally substituted with 1 to 5 groups independently selected from D and halo, and the remainder of the variables are as described in embodiments (1) and (2).

In addition to embodiments (1) through (3) in the preceding paragraphs, the compounds of Formula (I) or (I-a) or (I-b), or pharmaceutically acceptable salts thereof, include those in which:

(4) (A) the compound is of Formula (IVa) or (IVa-a) or (IVa-b):

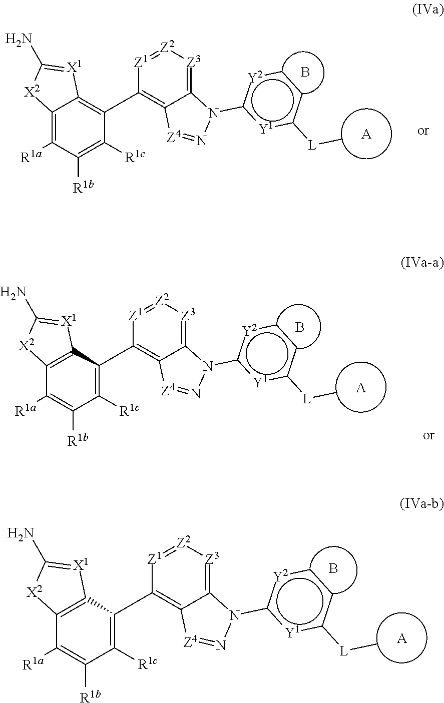

or a pharmaceutically acceptable salt thereof, and the remainder of the variables are as described in embodiment (1);
(B) the compound is of Formula (IVb) or (IVb-a) or (IVb-b):

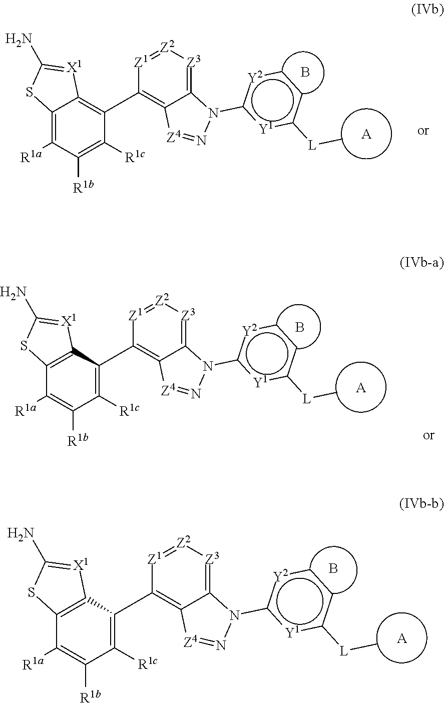

or a pharmaceutically acceptable salt thereof, and the remainder of the variables are as described in embodiment (1);
(C) the compound is of Formula (IVc) or (IVc-a) or (IVc-b):

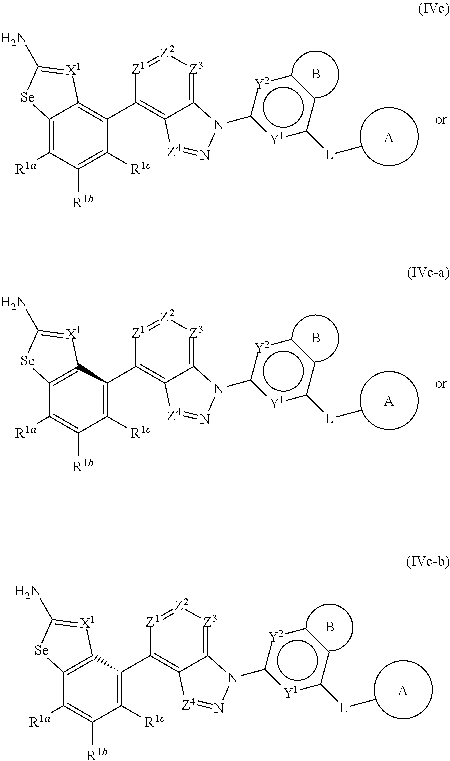

or a pharmaceutically acceptable salt thereof, and the remainder of the variables are as described in embodiment (1);
(D) the compound is of Formula (IVd) or (IVd-a) or (IVd-b):

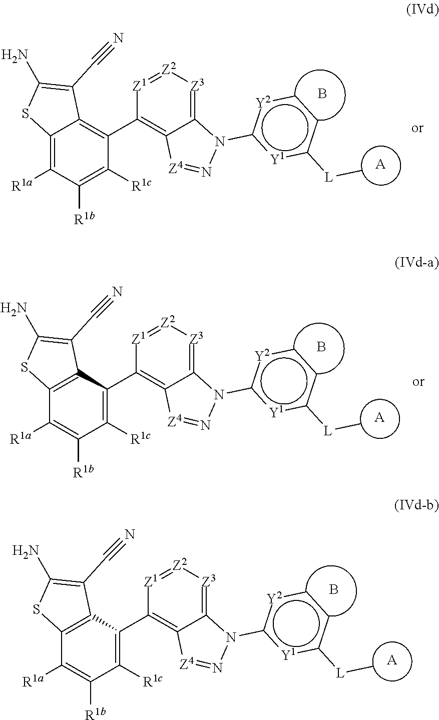

or a pharmaceutically acceptable salt thereof, and the remainder of the variables are as described in embodiment (1); or
(E) the compound is of Formula (IVe) or (IVe-a) or (IVe-b):

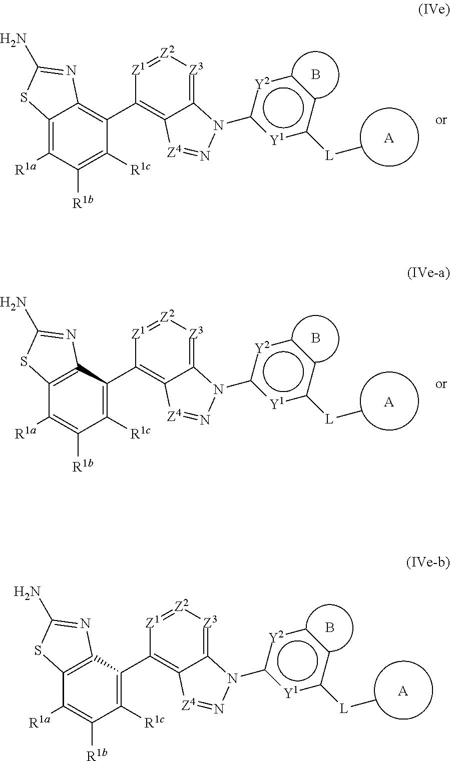

or a pharmaceutically acceptable salt thereof, and the remainder of the variables are as described in embodiment (1);
(F) the compound is of Formula (IVf) or (IVf-a) or (IVf-b):

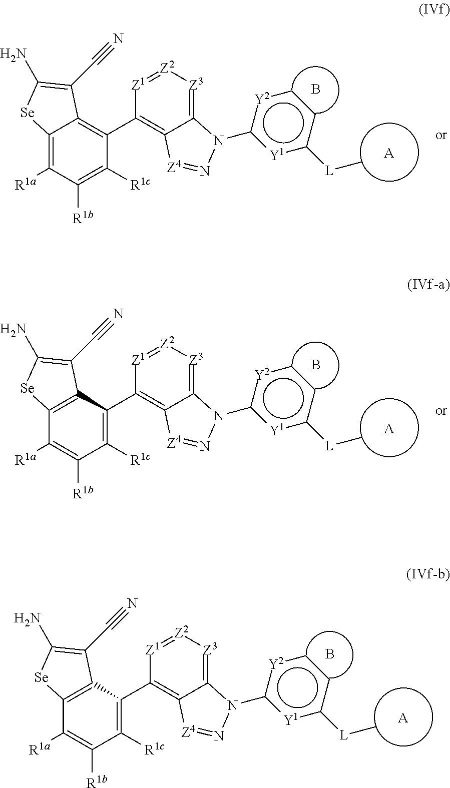

or a pharmaceutically acceptable salt thereof, and the remainder of the variables are as described in embodiment (1);
(G) the compound is of Formula (IVg) or (IVg-a) or (IVg-b):

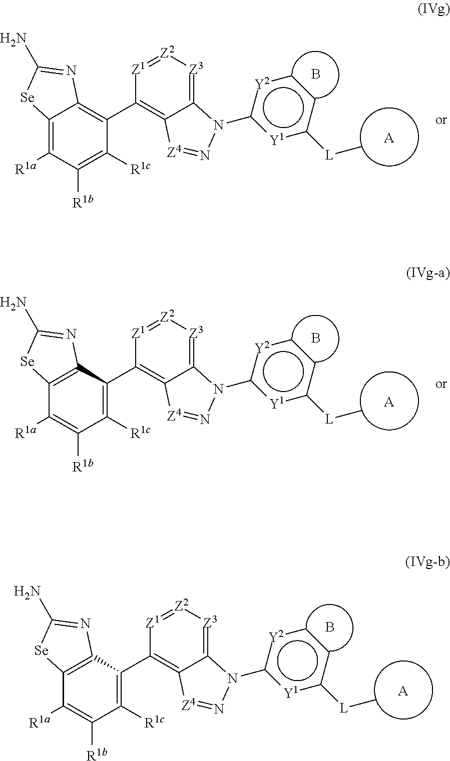

or a pharmaceutically acceptable salt thereof, and the remainder of the variables are as described in embodiment (1);
(H) the compound is of Formula (IVh) or (IVh-a) or (IVh-b):

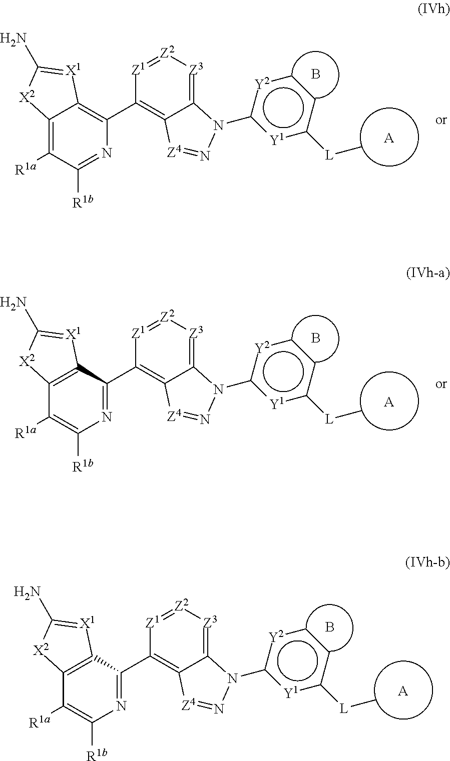

or a pharmaceutically acceptable salt thereof, and the remainder of the variables are as described in embodiment (1);
(I) the compound is of Formula (IVi) or (IVi-a) or (IVi-b):

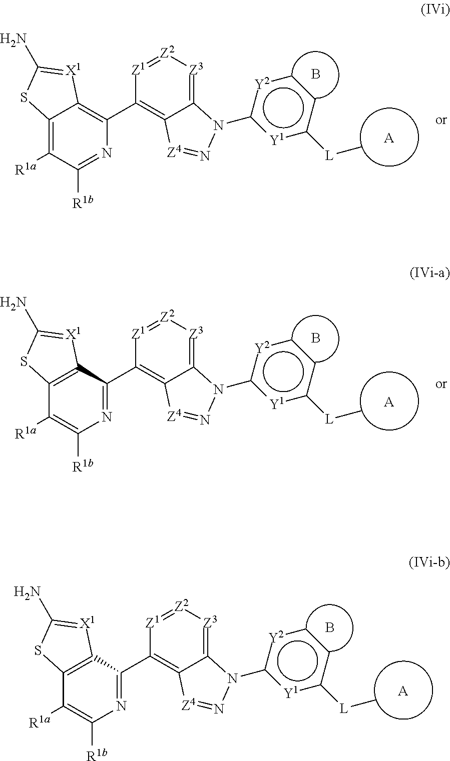

or a pharmaceutically acceptable salt thereof, and the remainder of the variables are as described in embodiment (1);
(J) the compound is of Formula (IVj) or (IVj-a) or (IVj-b):

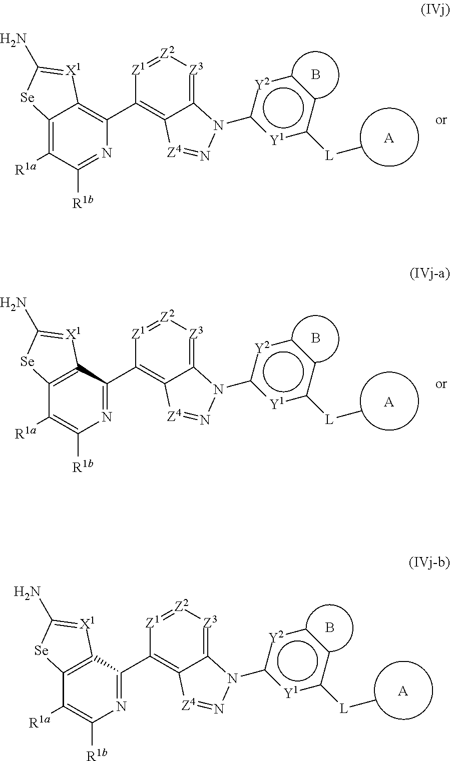

or a pharmaceutically acceptable salt thereof, and the remainder of the variables are as described in embodiment (1);
(K) the compound is of Formula (IVk) or (IVk-a) or (IVk-b):

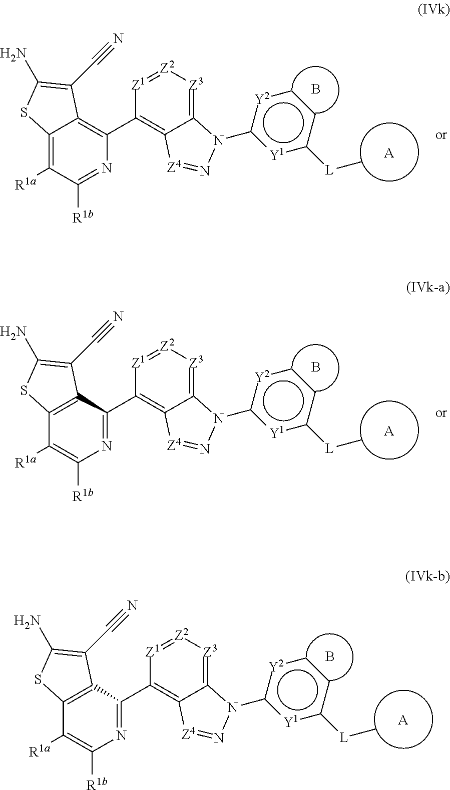

or a pharmaceutically acceptable salt thereof, and the remainder of the variables are as described in embodiment (1);
(L) the compound is of Formula (IVl) or (IVl-a) or (IVl-b):

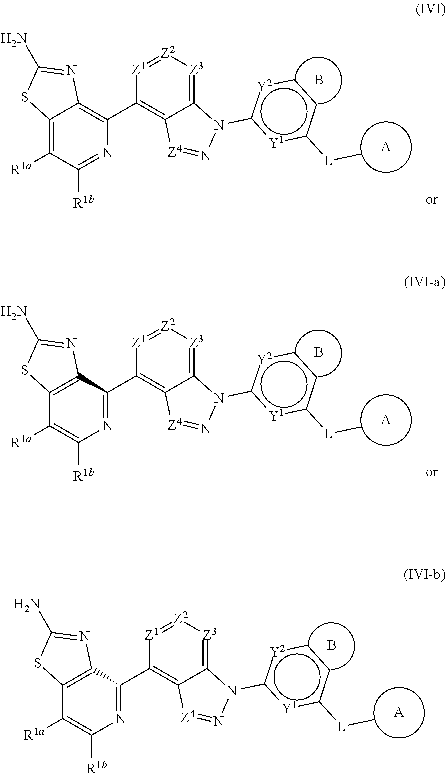

or a pharmaceutically acceptable salt thereof, and the remainder of the variables are as described in embodiment (1);
(M) the compound is of Formula (IVm) or (IVm-a) or (IVm-b):

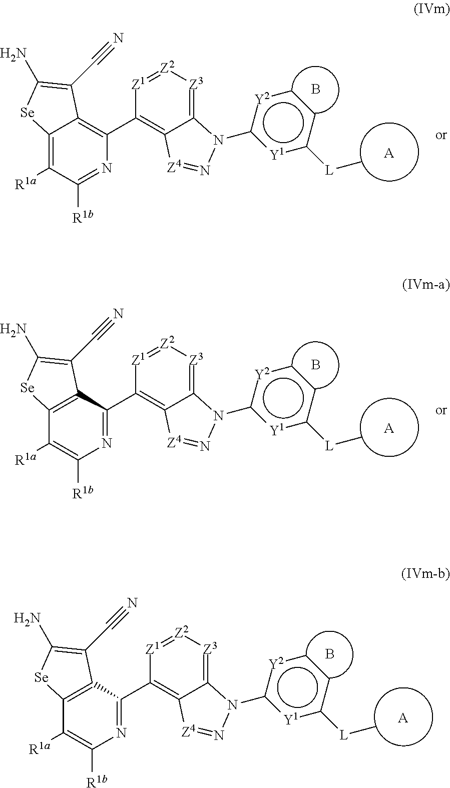

or a pharmaceutically acceptable salt thereof, and the remainder of the variables are as described in embodiment (1);
(N) the compound is of Formula (IVn) or (IVn-a) or (IVn-b):

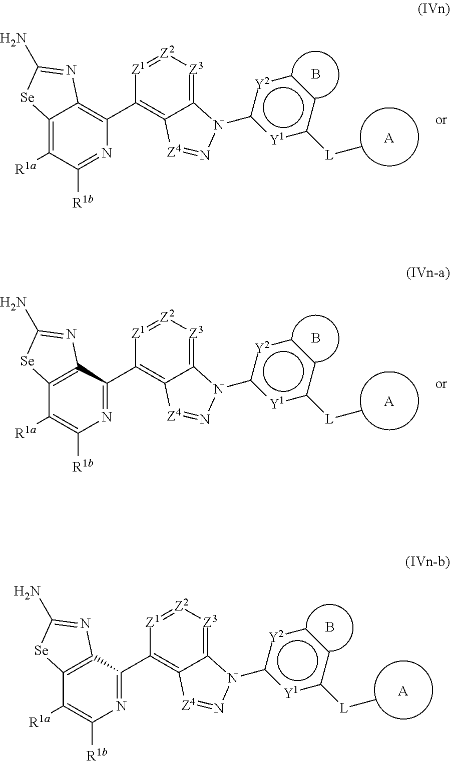

or a pharmaceutically acceptable salt thereof, and the remainder of the variables are as described in embodiment (1).

In addition to embodiments (1) through (4) in the preceding paragraphs, the compounds of Formula (I), (I-a), (I-b), (II), (II-a), (II-b), (III), (III-a), (III-b), (IVa), (IVa-a), (IVa-b), (IVb), (IVb-a), (IVb-b), (IVc), (IVc-a), (IVc-b), (IVd), (IVd-a), (IVd-b), (IVe), (IVe-a), (IVe-b), (IVf), (IVf-a), (IVf-b), (IVg), (IVg-a), (IVg-b), (IVh), (IVh-a), (IVh-b), (IVi), (IVi-a), (IVi-b), (IVj), (IVj-a), (IVj-b), (IVk), (IVk-a), (IVk-b), (IVl), (IVl-a), (IVl-b), (IVm), (IVm-a), (IVm-b), (IVn), (IVn-a), or (IVn-b), or pharmaceutically acceptable salts thereof, include those in which:

(5) R^{1a} and R^{1c} are each independently selected from H, halo, and C₁-C₄ alkyl and R^{1b} is H, and the remainder of the variables are as described in embodiments (1) through (3).

In addition to embodiments (1) through (5) in the preceding paragraphs, the compounds of Formula (I), (I-a), (I-b), (II), (II-a), (II-b), (III), (III-a), (III-b), (IVa), (IVa-a), (IVa-b), (IVb), (IVb-a), (IVb-b), (IVc), (IVc-a), (IVc-b), (IVd), (IVd-a), (IVd-b), (IVe), (IVe-a), (IVe-b), (IVf), (IVf-a), (IVf-b), (IVg), (IVg-a), (IVg-b), (IVh), (IVh-a), (IVh-b), (IVi), (IVi-a), (IVi-b), (IVj), (IVj-a), (IVj-b), (IVk), (IVk-a), (IVk-b), (IVl), (IVl-a), (IVl-b), (IVm), (IVm-a), (IVm-b), (IVn), (IVn-a), or (IVn-b), or pharmaceutically acceptable salts thereof, include those in which:

(6) (A) R^{1a} is selected from H, halo, and C₁-C₄ alkyl and R^{1b} is H, and the remainder of the variables are as described in embodiments (1) through (3) and (5);
(B) R^{1a} is H and R^{1b} is H, and the remainder of the variables are as described in embodiments (1) through (3) and (5);
(C) R^{1a} is halo and R^{1b} is H, and the remainder of the variables are as described in embodiments (1) through (3) and (5);
(D) R^{1a} is C₁-C₄ alkyl and R^{1b} is H, and the remainder of the variables are as described in embodiments (1) through (3) and (5).

In addition to embodiments (1) through (6) in the preceding paragraphs, the compounds of Formula (I), (I-a), (I-b), (II), (II-a), (II-b), (III), (III-a), (III-b), (IVa), (IVa-a), (IVa-b), (IVb), (IVb-a), (IVb-b), (IVc), (IVc-a), (IVc-b), (IVd), (IVd-a), (IVd-b), (IVe), (IVe-a), (IVe-b), (IVf), (IVf-a), (IVf-b), (IVg), (IVg-a), (IVg-b), (IVh), (IVh-a), (IVh-b), (IVi), (IVi-a), (IVi-b), (IVj), (IVj-a), (IVj-b), (IVk), (IVk-a), (IVk-b), (IVl), (IVl-a), (IVl-b), (IVm), (IVm-a), (IVm-b), (IVn), (IVn-a), or (IVn-b), or pharmaceutically acceptable salts thereof, include those in which:

(7) Z¹ is CR^{a}, Z² is CR^{b}, and Z³ is CR^{c} or N;
Z⁴ is CR^{d} or N;
Y¹ is N;
Y² is N;
L is selected from a bond and O(CR^{La}R^{Lb})ₘ₂—;
R^{La} and R^{Lb} are each independently selected from H, D, and C₁-C₄ alkyl, wherein the alkyl is optionally substituted with 1 to 4 groups independently selected from D and halo;
m2 is selected from 0, 1, and 2;
Ring A is a C₃-C₈ cycloalkyl or 3 to 10 membered heterocyclyl ring, wherein the cycloalkyl or heterocyclyl ring are optionally substituted with 1 to 4 groups independently selected from D, halo, ═O, OR^{g}, N(R^{g})₂, and C₁-C₄ alkyl, and wherein the alkyl is optionally substituted with 1 to 4 groups independently selected from D and halo, wherein the heterocyclyl ring comprises 1 or 2 heteroatoms selected from N, NR^{g}, and O;
Ring B is a C₃-C₈ cycloalkyl or 5 to 12 membered heterocyclyl, wherein the cycloalkyl or heterocyclyl are optionally substituted with 1 to 4 groups independently selected from D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂, wherein the heterocyclyl comprises 1 or 2 heteroatoms selected from N, NR^{g}, and O; or
R^{a}, R^{b}, R^{c} and R^{d} are each independently selected from H, D, halo, OR^{g}, N(R^{g})₂, and C₁-C₄ alkyl, wherein the alkyl is optionally substituted with 1 to 4 groups independently selected from D, halo, ═O, OR^{g}, and N(R^{g})₂; or
R^{a} and R^{b}, together with the carbon atoms to which they are attached, form a cyclopentyl, cyclohexyl, or a 5 to 6 membered heterocyclyl, wherein the cyclopentyl, cyclohexyl, or 5 to 6 membered heterocyclyl are optionally substituted with 1 to 3 groups independently selected from D, halo, C₁-C₄ alkyl, and C₁-C₄ alkoxy, wherein the alkyl and alkoxy are optionally substituted with 1 to 3 groups independently selected from D, halo and ORS, and the heterocyclyl comprises 1 or 2 heteroatoms selected from NR^{g} and O; or
R^{b} and R^{c}, together with the carbon atoms to which they are attached, form a cyclopentyl, cyclohexyl, or a 5 to 6 membered heterocyclyl, wherein the cyclopentyl, cyclohexyl, or 5 to 6 membered heterocyclyl are optionally substituted with 1 to 3 groups independently selected from D, halo, C₁-C₄ alkyl, and C₁-C₄ alkoxy, wherein the alkyl and alkoxy are optionally substituted with 1 to 3 groups independently selected from D, halo, and OR^{g}, and the heterocyclyl comprises 1 or 2 heteroatoms selected from NR^{g} and O; and
R^{g} is each independently selected from H and C₁-C₄ alkyl, wherein the alkyl is optionally substituted with 1 to 4 groups independently selected from D and halo, and the remainder of the variables are as described in embodiments (1) through (3), (5), and (6).

In addition to embodiment (1) through (7) in the preceding paragraphs, the compounds of Formula (I) or (I-a) or (I-b), or pharmaceutically acceptable salts thereof, include those in which:

(8) (A) the compound is of Formula (Va) or (Va-a) or (Va-b):

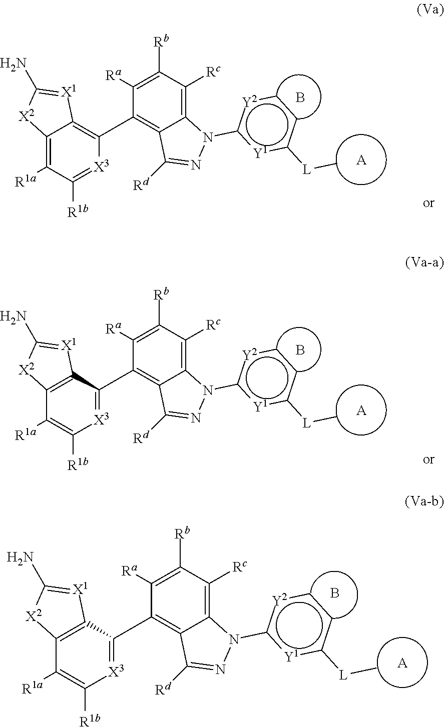

or a pharmaceutically acceptable salt thereof, and the remainder of the variables are as described in embodiment (1);
(B) the compound is of Formula (Vb) or (Vb-a) or (Vb-b):

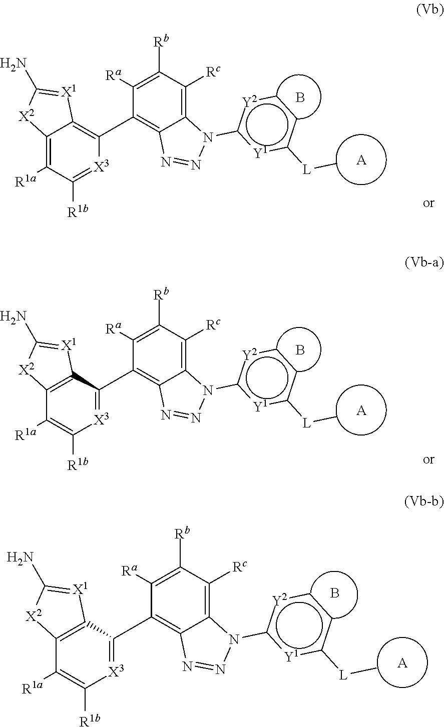

or a pharmaceutically acceptable salt thereof, and the remainder of the variables are as described in embodiment (1);
(C) the compound is of Formula (Vc) or (Vc-a) or (Vc-b):

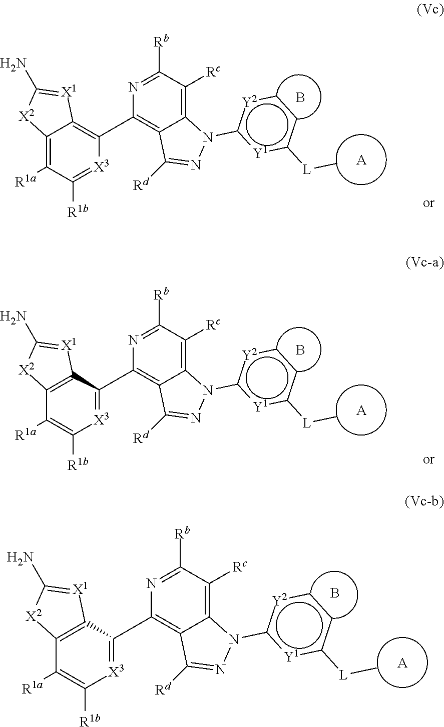

or a pharmaceutically acceptable salt thereof, and the remainder of the variables are as described in embodiment (1);
(D) the compound is of Formula (Vd) or (Vd-a) or (Vd-b):

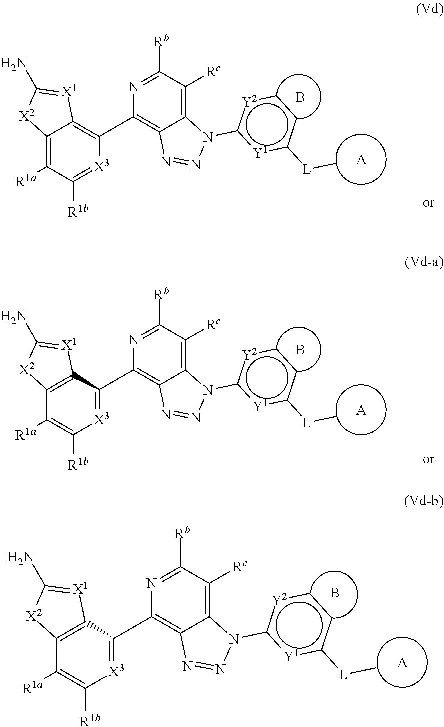

or a pharmaceutically acceptable salt thereof, and the remainder of the variables are as described in embodiment (1);
(E) the compound is of Formula (Ve) or (Ve-a) or (Ve-b):

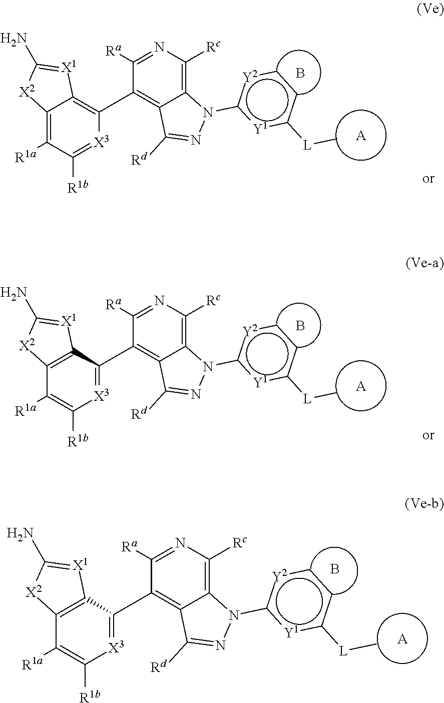

or a pharmaceutically acceptable salt thereof, and the remainder of the variables are as described in embodiment (1);
(F) the compound is of Formula (Vf) or (Vf-a) or (Vf-b):

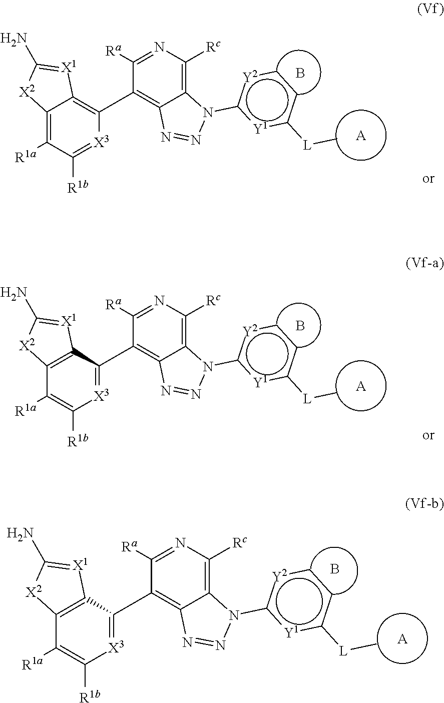

or a pharmaceutically acceptable salt thereof, and the remainder of the variables are as described in embodiment (1);
(G) the compound is of Formula (Vg) or (Vg-a) or (Vg-b):

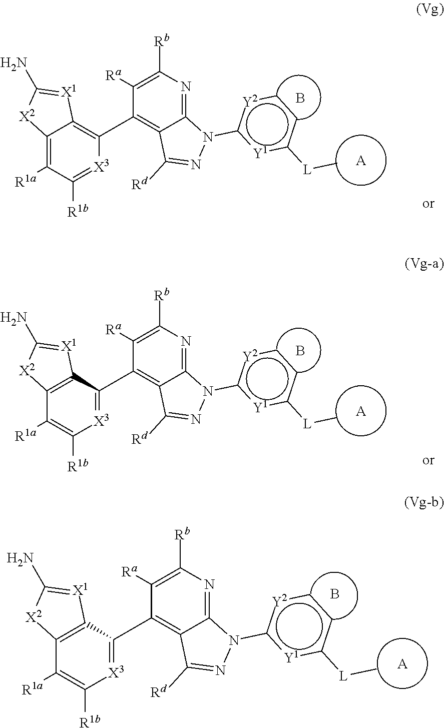

or a pharmaceutically acceptable salt thereof, and the remainder of the variables are as described in embodiment (1);
(H) the compound is of Formula (Vh) or (Vh-a) or (Vh-b):

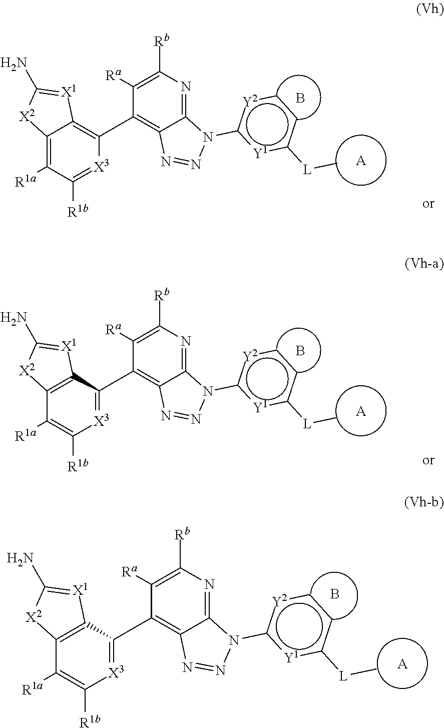

or a pharmaceutically acceptable salt thereof, and the remainder of the variables are as described in embodiment (1).

In addition to embodiments (1) and (8) in the preceding paragraphs, the compounds of Formula (I), (I-a), (I-b), (Va), (Va-a), (Va-b), (Vb), (Vb-a), (Vb-b), (Vc), (Vc-a), (Vc-b), (Vd), (Vd-a), (Vd-b), (Ve), (Ve-a), (Ve-b), (Vf), (Vf-a), (Vf-b), (Vg), (Vg-a), (Vg-b), (Vh), (Vh-a), or (Vh-b), or pharmaceutically acceptable salts thereof, include those in which:

### (9) X¹ is N or C—CN;

X² is S or Se;
X³ is N or CR^{1c};
L is selected from a bond and O(CR^{La}R^{Lb})ₘ₂—;
R^{La} and R^{Lb} are each independently selected from H, D, and C₁-C₄ alkyl, wherein the alkyl is optionally substituted with 1 to 4 groups independently selected from D and halo; m2 is selected from 0, 1, and 2;
Ring A is a C₃-C₈ cycloalkyl or 3 to 10 membered heterocyclyl ring, wherein the cycloalkyl or heterocyclyl ring are optionally substituted with 1 to 4 groups independently selected from D, halo, ═O, OR^{g}, N(R^{g})₂, and C₁-C₄ alkyl, and wherein the alkyl is optionally substituted with 1 to 4 groups independently selected from D and halo, wherein the heterocyclyl ring comprises 1 or 2 heteroatoms selected from N, NR^{g}, and O;
Ring B is a C₃-C₈ cycloalkyl or 5 to 12 membered heterocyclyl, wherein the cycloalkyl or heterocyclyl are optionally substituted with 1 to 4 groups independently selected from D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂, wherein the heterocyclyl comprises 1 or 2 heteroatoms selected from N, NR^{g}, and O; or
R^{1a} and R^{1c} are each independently selected from H, halo, and C₁-C₄ alkyl and R^{1b} is H;
R^{a}, R^{b}, R^{c} and R^{d} are each independently selected from H, D, halo, OR^{g}, N(R^{g})₂, and C₁-C₄ alkyl, wherein the alkyl is optionally substituted with 1 to 4 groups independently selected from D, halo, ═O, OR^{g}, and N(R^{g})₂; or
R^{a} and R^{b}, together with the carbon atoms to which they are attached, form a cyclopentyl, cyclohexyl, or a 5 to 6 membered heterocyclyl, wherein the cyclopentyl, cyclohexyl, or 5 to 6 membered heterocyclyl are optionally substituted with 1 to 3 groups independently selected from D, halo, C₁-C₄ alkyl, and C₁-C₄ alkoxy, wherein the alkyl and alkoxy are optionally substituted with 1 to 3 groups independently selected from D, halo, and OR^{g}, and the heterocyclyl comprises 1 or 2 heteroatoms selected from NR^{g} and O; or
R^{b} and R^{c}, together with the carbon atoms to which they are attached, form a cyclopentyl, cyclohexyl, or a 5 to 6 membered heterocyclyl, wherein the cyclopentyl, cyclohexyl, or 5 to 6 membered heterocyclyl are optionally substituted with 1 to 3 groups independently selected from D, halo, C₁-C₄ alkyl, and C₁-C₄ alkoxy, wherein the alkyl and alkoxy are optionally substituted with 1 to 3 groups independently selected from D, halo, and OR^{g}, and the heterocyclyl comprises 1 or 2 heteroatoms selected from NR^{g} and O; and
R^{g} is each independently selected from H and C₁-C₄ alkyl, wherein the alkyl is optionally substituted with 1 to 4 groups independently selected from D and halo, and the remainder of the variables are as described in embodiment (1).

In addition to embodiments (1) through (9) in the preceding paragraphs, the compounds of Formula (I), (I-a), (I-b), (Va), (Va-a), (Va-b), (Vb), (Vb-a), (Vb-b), (Vc), (Vc-a), (Vc-b), (Vd), (Vd-a), (Vd-b), (Ve), (Ve-a), (Ve-b), (Vf), (Vf-a), (Vf-b), (Vg), (Vg-a), (Vg-b), (Vh), (Vh-a), or (Vh-b), or pharmaceutically acceptable salts thereof, include those in which:

(10) Y¹ is N and Y² is N, and the remainder of the variables are as described in embodiments (1) and (9).

In addition to embodiments (1) through (10) in the preceding paragraphs, the compounds of Formula (I) or (I-a) or (I-b), or pharmaceutically acceptable salts thereof, include those in which:

(11) the compound is of Formula (VIa) or (VIa-a) or (VIa-b):

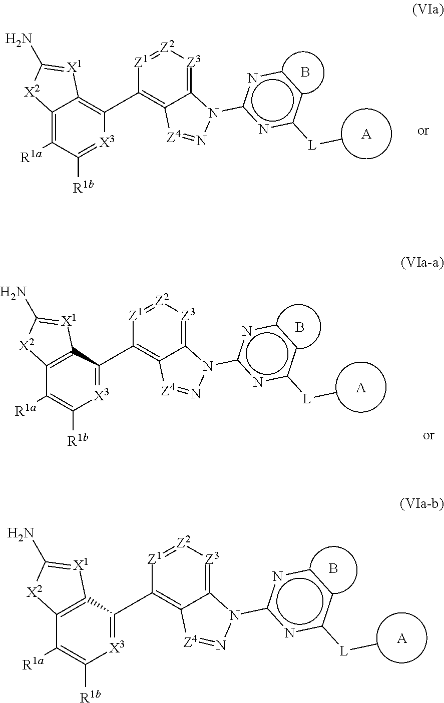

or a pharmaceutically acceptable salt thereof, wherein:
Ring B is a C₃-C₈ cycloalkyl or 5 to 12 membered heterocyclyl, wherein the cycloalkyl or heterocyclyl are optionally substituted with 1 to 4 groups independently selected from D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂, wherein the heterocyclyl comprises 1 or 2 heteroatoms selected from N, NR^{g}, and O, and the remainder of the variables are as described in embodiment (1).

In addition to embodiments (1) through (11) in the preceding paragraphs, the compounds of Formula (I) or (I-a) or (I-b), or pharmaceutically acceptable salts thereof, include those in which:

(12) the compound is of Formula (VIb) or (VIb-a) or (VIb-b):

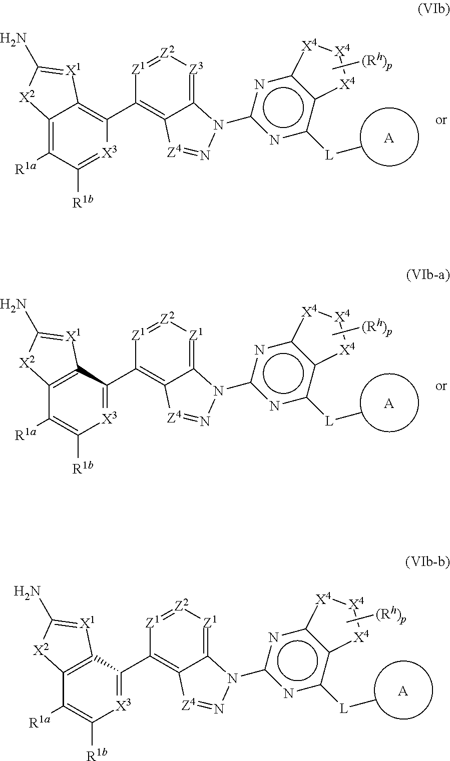

or a pharmaceutically acceptable salt thereof, wherein:
X⁴ is each independently selected from O, NR^{g}, and C(R^{h})₂, provided that at least one X⁴ is C(R^{h})₂;
R^{h} is each independently selected from H, D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂; or
2 R^{h}, along with the carbon atom to which they are attached, form a 4 to 6 membered heterocyclyl; and
p is selected from 0, 1, 2, 3, and 4, and the remainder of the variables are as described in embodiment (1).

In addition to embodiments (1) through (12) in the preceding paragraphs, the compounds of Formula (I) or (I-a) or (I-b), or pharmaceutically acceptable salts thereof, include those in which:

(13) the compound is of Formula (VIc) or (VIc-a) or (VIc-b):

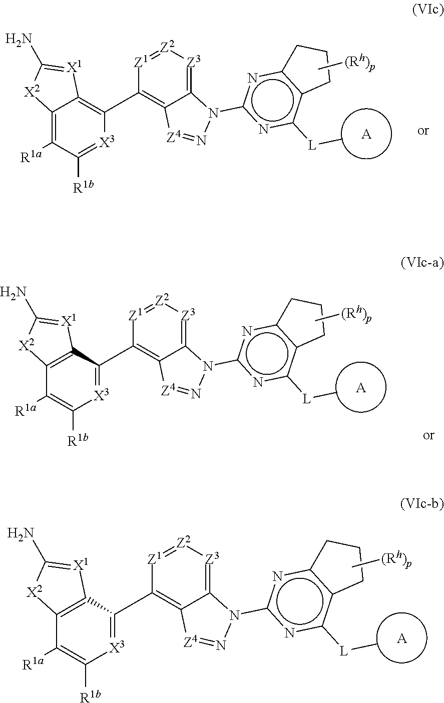

or a pharmaceutically acceptable salt thereof, wherein:
R^{h} is H, D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂; and
p is selected from 0, 1, 2, 3, and 4, and the remainder of the variables are as described in embodiment (1).

In addition to embodiments (1) through (13) in the preceding paragraphs, the compounds of Formula (I) or (I-a) or (I-b), or pharmaceutically acceptable salts thereof, include those in which:

(14) (A) the compound is of Formula (VId) or (VId-a) or (VId-b):

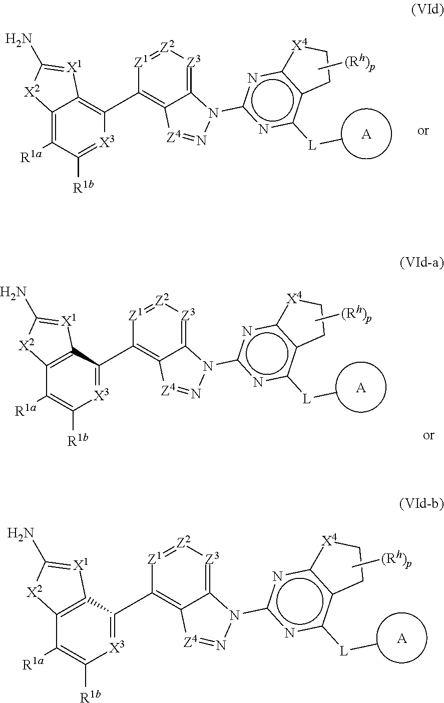

or a pharmaceutically acceptable salt thereof, wherein:
X⁴ is selected from O and NR^{g};
R^{h} is each independently selected from H, D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂; and
p is selected from 0, 1, 2, 3, and 4, and the remainder of the variables are as described in embodiment (1);
   (B) the compound is of Formula (VIe) or (VIe-a) or (VIe-b):

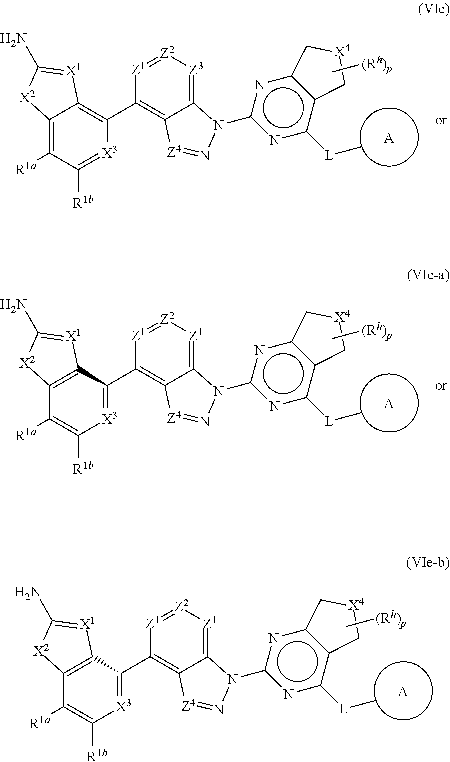

or a pharmaceutically acceptable salt thereof, wherein:
X⁴ is selected from O and NR^{g};
R^{h} is each independently selected from H, D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂; and
p is selected from 0, 1, 2, 3, and 4, and the remainder of the variables are as described in embodiment (1);
(C) the compound is of Formula (VIf) or (VIf-a) or (VIf-b):

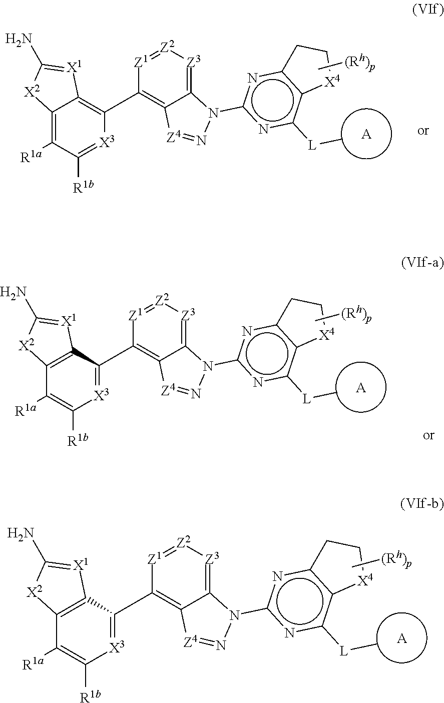

or a pharmaceutically acceptable salt thereof, wherein:
X⁴ is selected from O and NR^{g};
R^{h} is each independently selected from H, D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂; and
p is selected from 0, 1, 2, 3, and 4, and the remainder of the variables are as described in embodiment (1).

In addition to embodiments (1) through (14) in the preceding paragraphs, the compounds of Formula (I) or (I-a) or (I-b), or pharmaceutically acceptable salts thereof, include those in which:

(15) (A) the compound is of Formula (VIg) or (VIg-a) or (VIg-b):

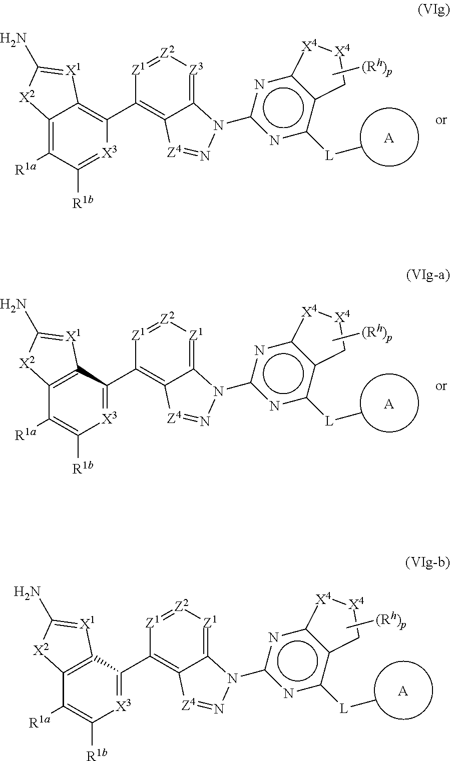

or a pharmaceutically acceptable salt thereof, wherein:
X⁴ is each independently selected from O and NR^{g};
R^{h} is each independently selected from H, D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂; and
p is selected from 0, 1, and 2, and the remainder of the variables are as described in embodiment (1);
   (B) the compound is of Formula (VIh) or (VIh-a) or (VIh-b):

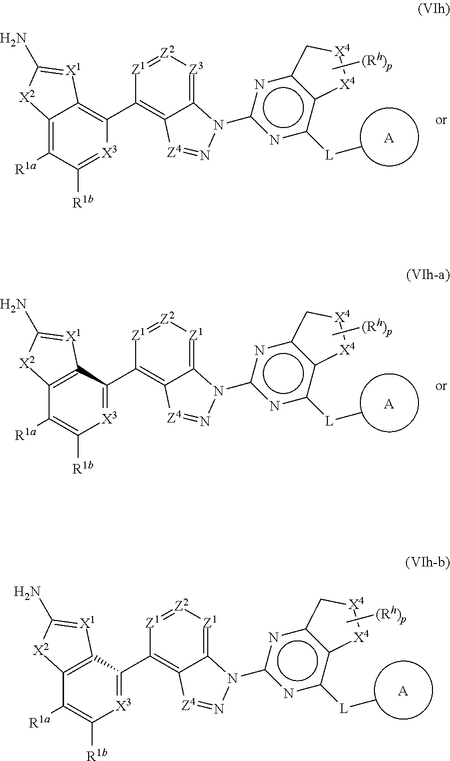

or a pharmaceutically acceptable salt thereof, wherein:
X⁴ is each independently selected from O and NR^{g};
R^{h} is each independently selected from H, D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂; and
p is selected from 0, 1, and 2, and the remainder of the variables are as described in embodiment (1);
   (C) the compound is of Formula (VIi) or (VIi-a) or (VIi-b):

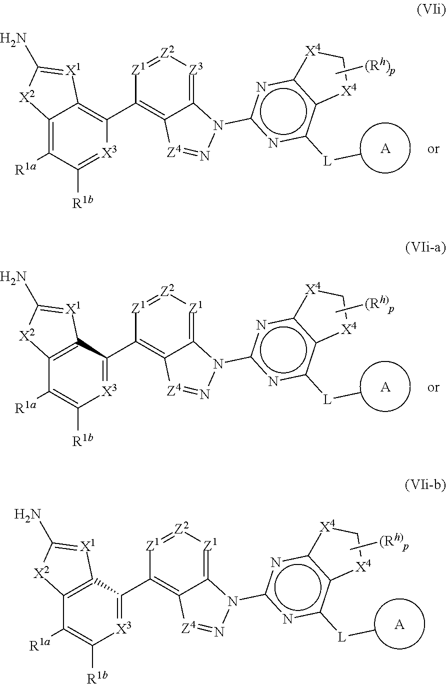

or a pharmaceutically acceptable salt thereof, wherein:
X⁴ is each independently selected from O and NR^{g};
R^{h} is each independently selected from H, D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂; and
p is selected from 0, 1, and 2, and the remainder of the variables are as described in embodiment (1).

In addition to embodiments (1) through (15) in the preceding paragraphs, the compounds of Formula (I) or (I-a) or (I-b), or pharmaceutically acceptable salts thereof, include those in which:

(16) the compound is of Formula (VIj) or (VIj-a) or (VIj-b):

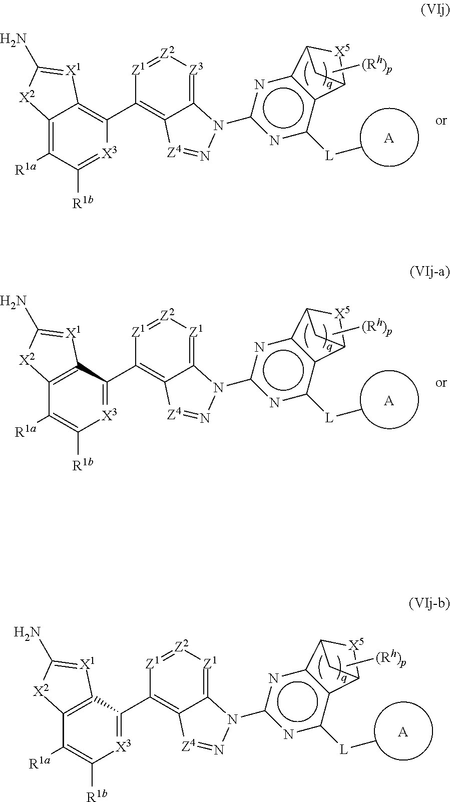

or a pharmaceutically acceptable salt thereof; wherein:
X⁵ is each independently selected from O and NR^{g},
R^{h} is each independently selected from D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂;
p is selected from 0, 1, 2, 3, and 4; and
q is selected from 1 and 2, and the remainder of the variables are as described in embodiment (1).

In addition to embodiments (1) through (16) in the preceding paragraphs, the compounds of Formula (I) or (I-a) or (I-b), or pharmaceutically acceptable salts thereof, include those in which:

(17) the compound is of Formula (VIk) or (VIk-a) or (VIk-b):

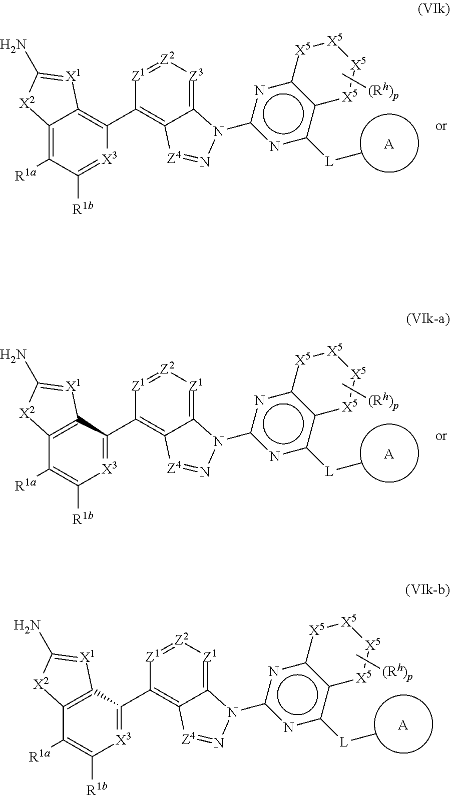

or a pharmaceutically acceptable salt thereof, wherein:
X⁵ is each independently selected from O, NR^{g}, and C(R^{h})₂, provided that at least two X⁵ are C(R^{h})₂;
R^{h} is each independently selected from H, D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂; or
2 R^{h}, along with the carbon atom to which they are attached, form a 4 to 6 membered heterocyclyl; and
p is selected from 0, 1, 2, 3, and 4, and the remainder of the variables are as described in embodiment (1).

In addition to embodiments (1) through (17) in the preceding paragraphs, the compounds of Formula (I) or (I-a) or (I-b), or pharmaceutically acceptable salts thereof, include those in which:

(18) the compound is of Formula (VII) or (VII-a) or (VII-b):

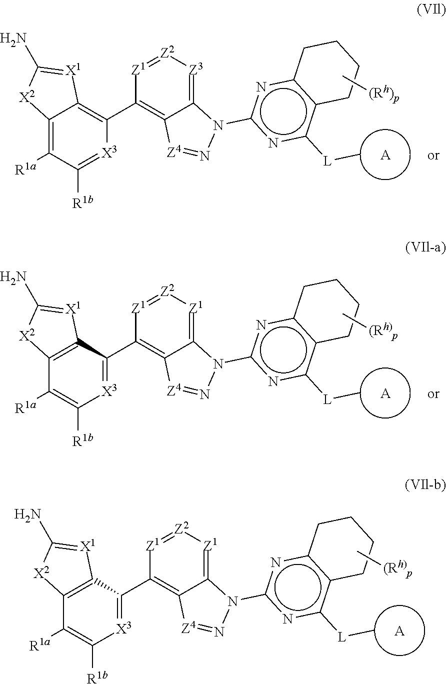

or a pharmaceutically acceptable salt thereof, wherein:
R^{h} is H, D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂; and
p is selected from 0, 1, 2, 3, and 4, and the remainder of the variables are as described in embodiment (1).

In addition to embodiments (1) through (18) in the preceding paragraphs, the compounds of Formula (I) or (I-a) or (I-b), or pharmaceutically acceptable salts thereof, include those in which:

(19) (A) the compound is of Formula (VIm) or (VIm-a) or (VIm-b):

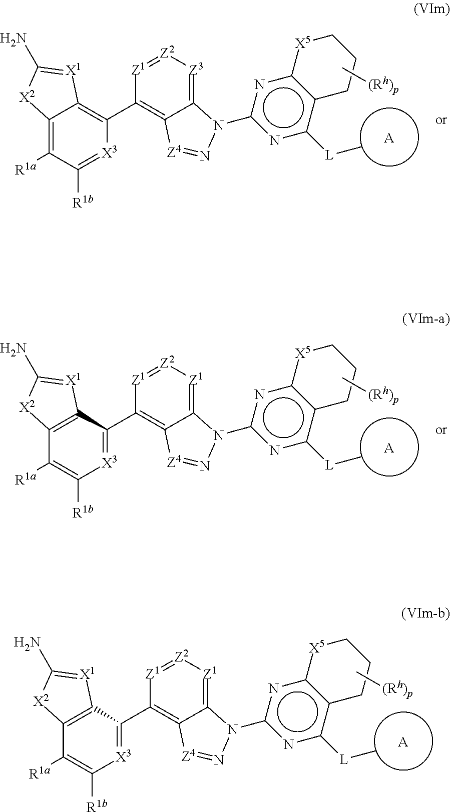

or a pharmaceutically acceptable salt thereof, wherein:
X⁵ is selected from O and NR^{g};
R^{h} is each independently selected from H, D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂; and
p is selected from 0, 1, 2, 3, and 4, and the remainder of the variables are as described in embodiment (1);
   (B) the compound is of Formula (VIn) or (VIn-a) or (VIn-b):

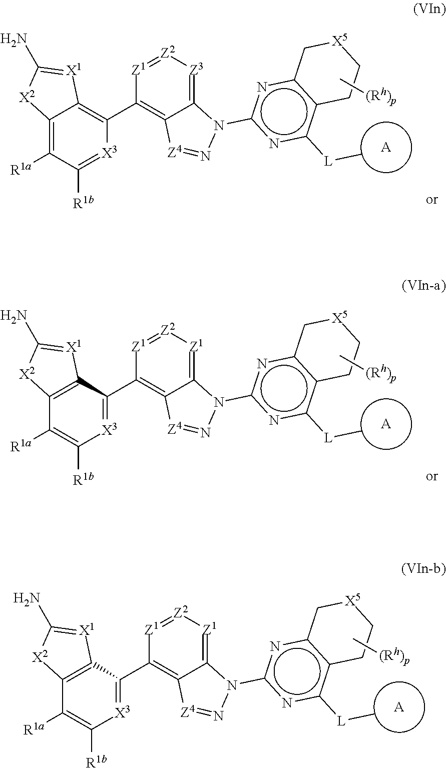

or a pharmaceutically acceptable salt thereof, wherein:
X⁵ is selected from O and NR^{g};
R^{h} is each independently selected from H, D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂; and
p is selected from 0, 1, 2, 3, and 4, and the remainder of the variables are as described in embodiment (1);
   (C) the compound is of Formula (VIo) or (VIo-a) or (VIo-b):

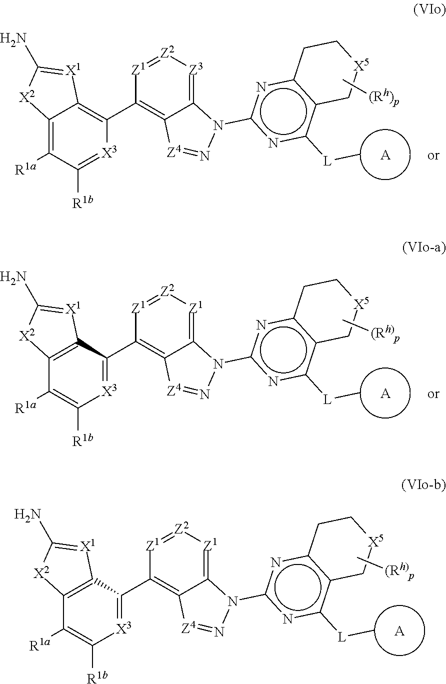

or a pharmaceutically acceptable salt thereof, wherein:
X⁵ is selected from O and NR^{g};
R^{h} is each independently selected from H, D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂; and
p is selected from 0, 1, 2, 3, and 4, and the remainder of the variables are as described in embodiment (1);
   (D) the compound is of Formula (VIp) or (VIp-a) or (VIp-b):

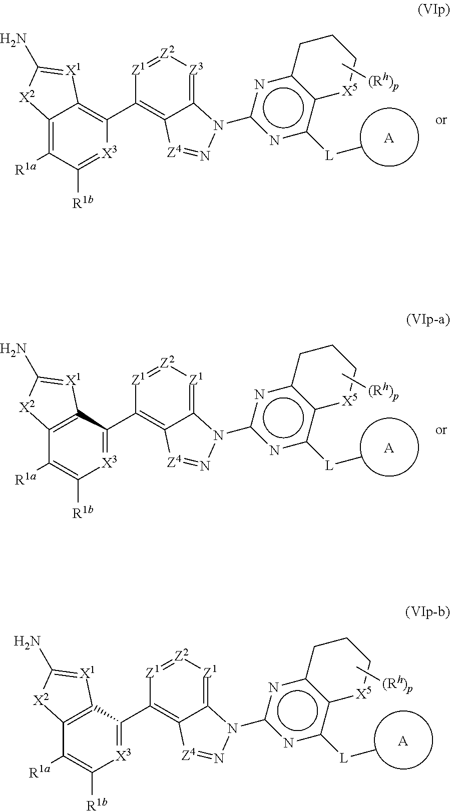

or a pharmaceutically acceptable salt thereof, wherein:
X⁵ is selected from O and NR^{g};
R^{h} is each independently selected from H, D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂; and
p is selected from 0, 1, 2, 3, and 4, and the remainder of the variables are as described in embodiment (1).

In addition to embodiments (1) through (19) in the preceding paragraphs, the compounds of Formula (I) or (I-a) or (I-b), or pharmaceutically acceptable salts thereof, include those in which:

(20) the compound is of Formula (VIq) or (VIq-a) or (VIq-b):

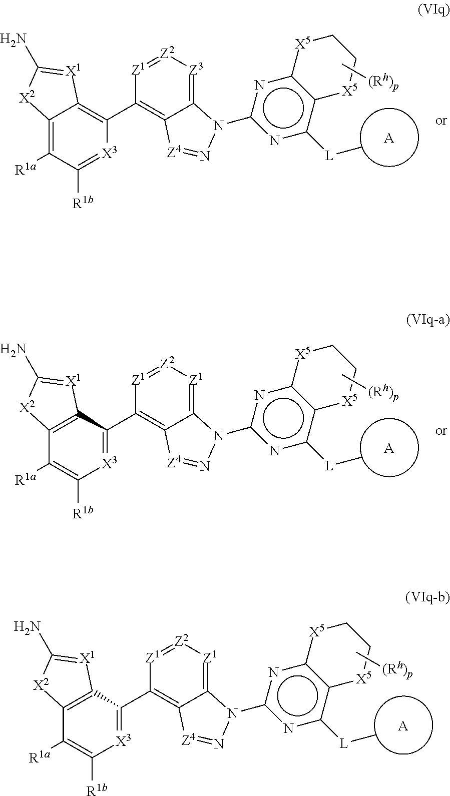

or a pharmaceutically acceptable salt thereof, wherein:
X⁵ is each independently selected from O and NR^{g};
R^{h} is each independently selected from H, D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂; and
p is selected from 0, 1, 2, 3, and 4, and the remainder of the variables are as described in embodiment (1).

In addition to embodiments (1) through (20) in the preceding paragraphs, the compounds of Formula (I) or (I-a) or (I-b), or pharmaceutically acceptable salts thereof, include those in which:

(21) (A) the compound is of Formula (VIr) or (VIr-a) or (VIr-b):

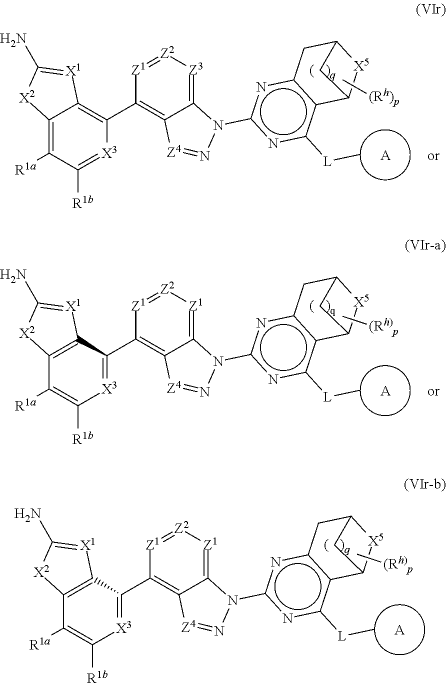

or a pharmaceutically acceptable salt thereof; wherein:
X⁵ is each independently selected from O and NR^{g},
R^{h} is each independently selected from D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂;
p is selected from 0, 1, 2, 3, and 4; and
q is selected from 1 and 2, and the remainder of the variables are as described in embodiment (1);
   (B) the compound is of Formula (VIs) or (VIs-a) or (VIs-b):

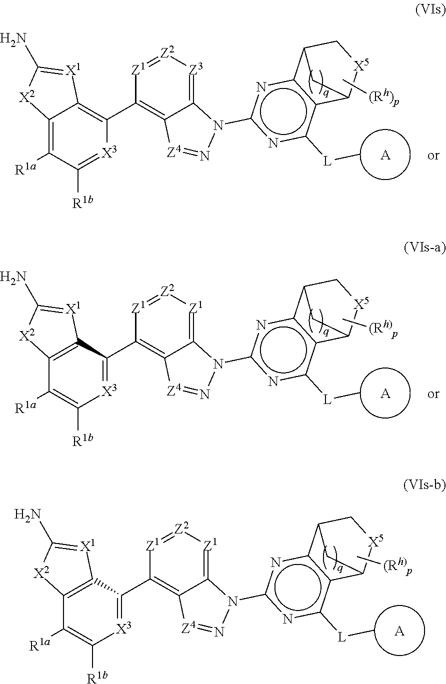

or a pharmaceutically acceptable salt thereof; wherein:
X⁵ is each independently selected from O and NR^{g},
R^{h} is each independently selected from D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂;
p is selected from 0, 1, 2, 3, and 4; and
q is selected from 1 and 2, and the remainder of the variables are as described in embodiment (1).

In addition to embodiments (1) through (21) in the preceding paragraphs, the compounds of Formula (I) or (I-a) or (I-b), or pharmaceutically acceptable salts thereof, include those in which:

(22) the compound is of Formula (VIt) or (VIt-a) or (VIt-b):

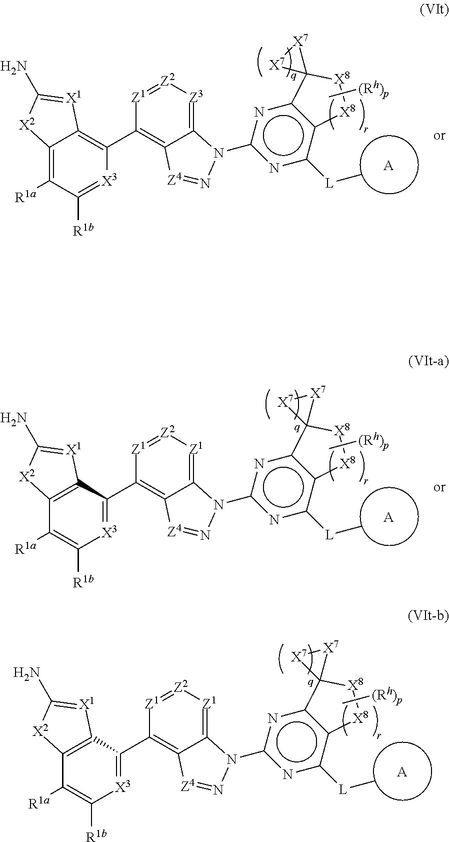

or a pharmaceutically acceptable salt thereof; wherein:
X⁷ is each independently selected from O, NR^{g}, and C(R^{h})₂, provided that only one X⁷ is O or NR^{g};
X⁸ is each independently selected from O, NR^{g}, and C(R^{h})₂, provided that only one X⁸ is O or NR^{g};
provided at least one of X⁷ and X⁸ is not C(R^{h})₂;
R^{h} is each independently selected from D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂;
p is selected from 0, 1, 2, 3, and 4;
q is selected from 1, 2, 3, and 4; and
r is selected from 1, 2, and 3, and the remainder of the variables are as described in embodiment (1).

In addition to embodiments (1) through (22) in the preceding paragraphs, the compounds of Formula (I) or (I-a) or (I-b), or pharmaceutically acceptable salts thereof, include those in which:

(23) the compound is of Formula (VIu) or (VIu-a) or (VIu-b):

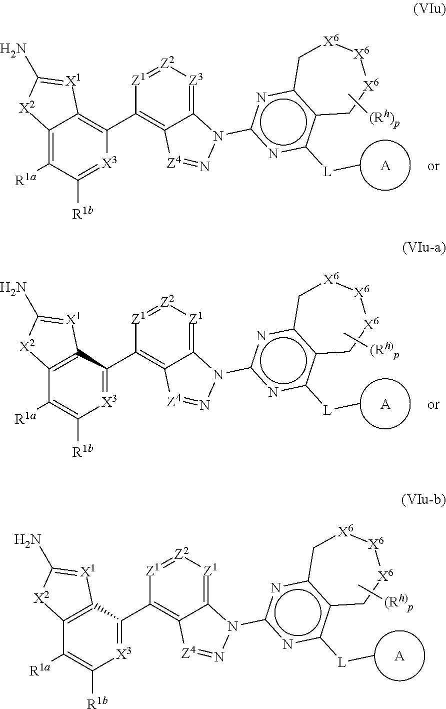

or a pharmaceutically acceptable salt thereof, wherein:
X⁶ is each independently selected from O, NR^{g}, or C(R^{h})₂, provided that at least two X⁶ are C(R^{h})₂;
R^{h} is each independently selected from H, D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂; and
p is selected from 0, 1, 2, 3, and 4, and the remainder of the variables are as described in embodiment (1).

In addition to embodiments (1) through (23) in the preceding paragraphs, the compounds of Formula (I) or (I-a) or (I-b), or pharmaceutically acceptable salts thereof, include those in which:

(24) (A) the compound is of Formula (VIv) or (VIv-a) or (VIv-b):

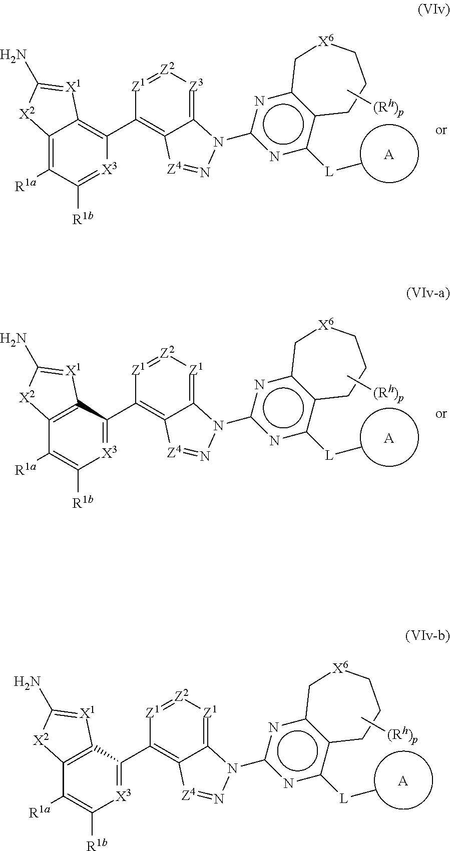

or a pharmaceutically acceptable salt thereof, wherein:
X⁶ is each independently selected from O and NR^{g};
R^{h} is each independently selected from D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂; and
p is selected from 0, 1, 2, 3, and 4, and the remainder of the variables are as described in embodiment (1);
   (B) the compound is of Formula (VIw) or (VIw-a) or (VIw-b):

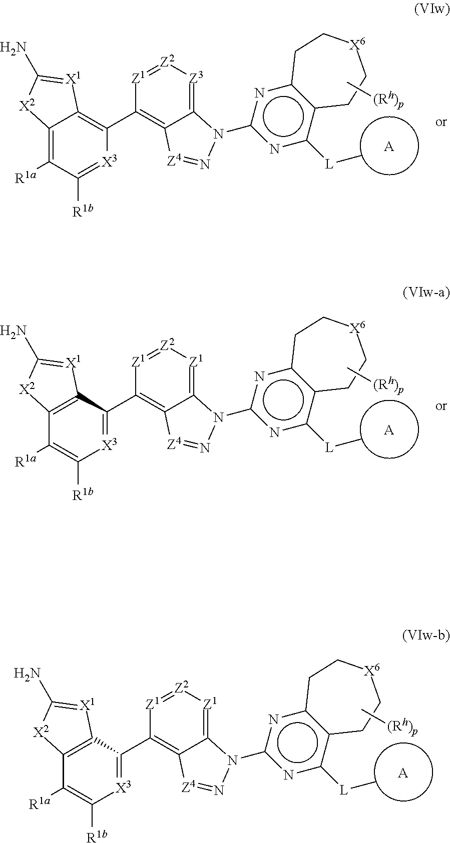

or a pharmaceutically acceptable salt thereof, wherein:
X⁶ is each independently selected from O and NR^{g};
R^{h} is each independently selected from D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂; and
p is selected from 0, 1, 2, 3, and 4, and the remainder of the variables are as described in embodiment (1);
   (C) the compound is of Formula (VIx) or (VIx-a) or (VIx-b):

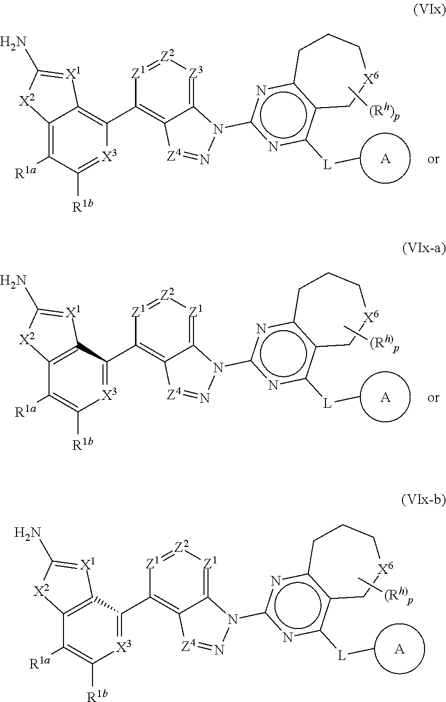

or a pharmaceutically acceptable salt thereof, wherein:
X⁶ is each independently selected from O and NR^{g};
R^{h} is each independently selected from D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂; and
p is selected from 0, 1, 2, 3, and 4, and the remainder of the variables are as described in embodiment (1).

In addition to embodiments (1) through (24) in the preceding paragraphs, the compounds of Formula (I) or (I-a) or (I-b), or pharmaceutically acceptable salts thereof, include those in which:

(25) (A) the compound is of Formula (VIy) or (VIy-a) or (VIy-b):

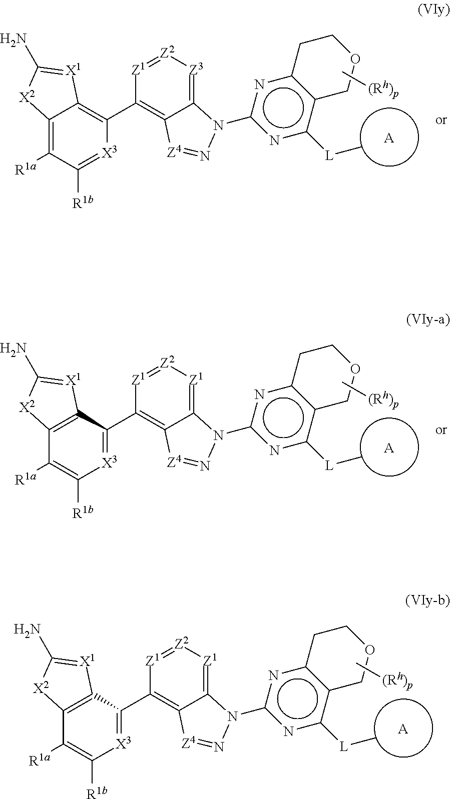

or a pharmaceutically acceptable salt thereof, wherein:
R^{h} is each independently selected from D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂; and
p is selected from 0, 1, 2, 3, and 4, and the remainder of the variables are as described in embodiment (1);
   (B) the compound is of Formula (VIz) or (VIz-a) or (VIz-b):

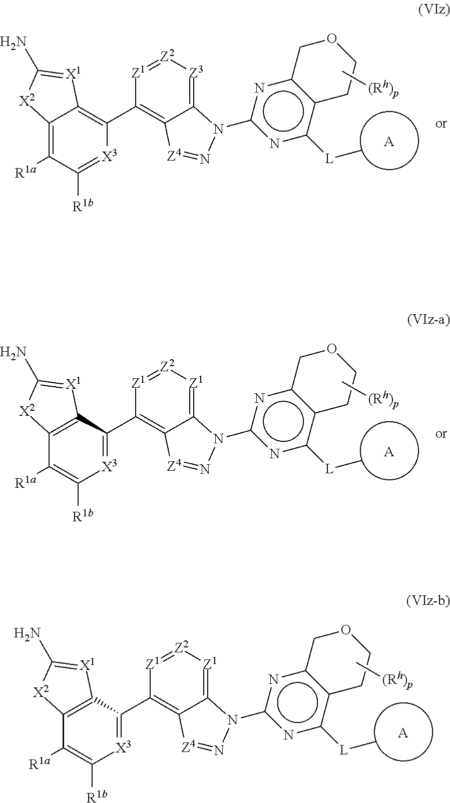

or a pharmaceutically acceptable salt thereof, wherein:
R^{h} is each independently selected from D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂; and
p is selected from 0, 1, 2, 3, and 4, and the remainder of the variables are as described in embodiment (1);
   (C) the compound is of Formula (VIaa) or (VIaa-a) or (VIaa-b):

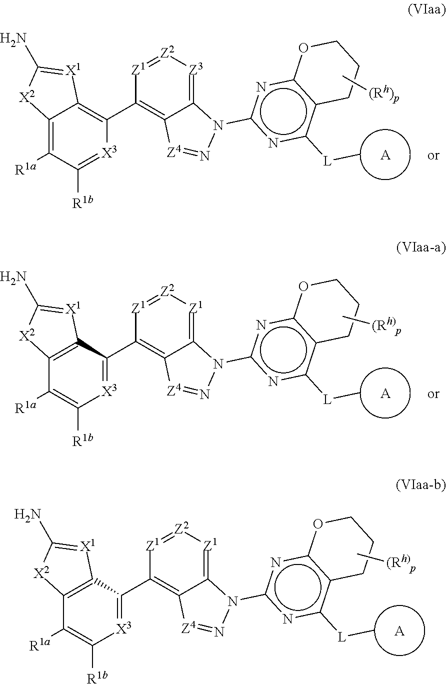

or a pharmaceutically acceptable salt thereof, wherein:
R^{h} is each independently selected from D, halo, ═O, OR^{g}, N(R^{g})₂, and C₁-C₄ alkyl, wherein the and N(R^{g})₂; and
p is selected from 0, 1, 2, 3, and 4, and the remainder of the variables are as described in embodiment (1);
   (D) the compound is of Formula (VIab) or (VIab-a) or (VIab-b):

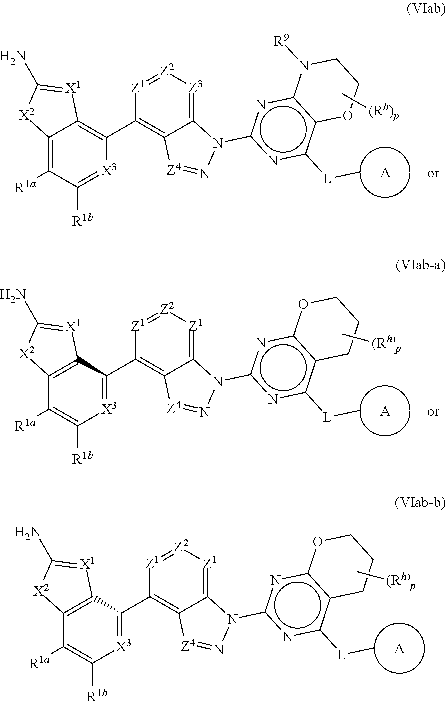

or a pharmaceutically acceptable salt thereof, wherein:
R^{h} is each independently selected from D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂; and
p is selected from 0, 1, 2, 3, and 4, and the remainder of the variables are as described in embodiment (1);
   (E) the compound is of Formula (VIac) or (VIac-a) or (VIac-b):

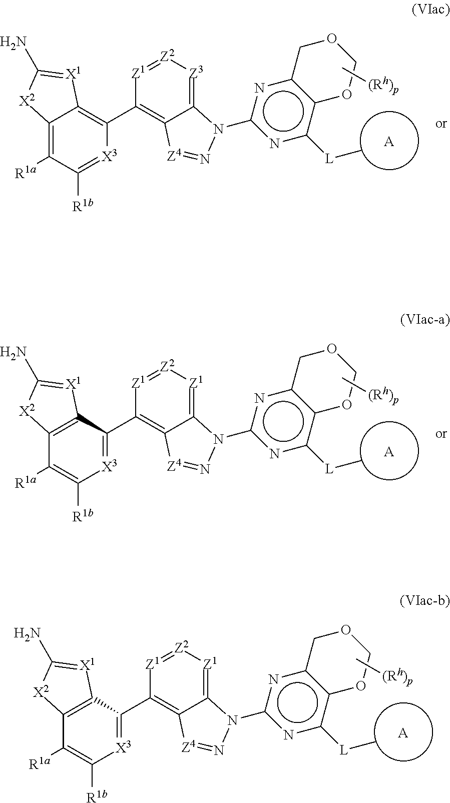

or a pharmaceutically acceptable salt thereof, wherein:
R^{h} is each independently selected from D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂; and
p is selected from 0, 1, 2, 3, and 4, and the remainder of the variables are as described in embodiment (1);
   (F) the compound is of Formula (VIad) or (VIad-a) or (VIad-b):

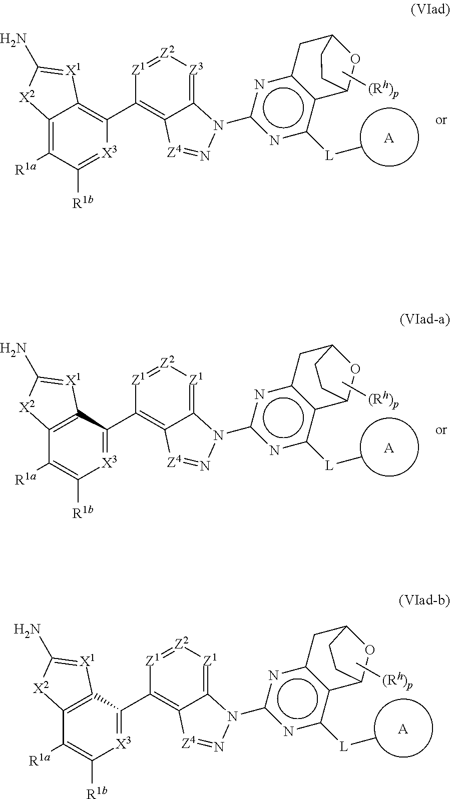

or a pharmaceutically acceptable salt thereof, wherein:
R^{h} is each independently selected from D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂; and
p is selected from 0, 1, 2, 3, and 4, and the remainder of the variables are as described in embodiment (1);
   (G) the compound is of Formula (VIae) or (VIae-a) or (VIae-b):

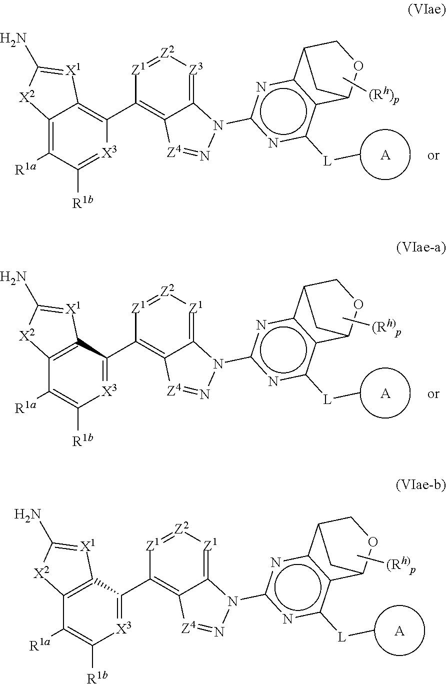

or a pharmaceutically acceptable salt thereof, wherein:
R^{h} is each independently selected from D, halo, ═O, OR^{g}, N(R^{g})₂, and C₁-C₄ alkyl, wherein the and N(R^{g})₂; and
p is selected from 0, 1, 2, 3, and 4, and the remainder of the variables are as described in embodiment (1);
   (H) the compound is of Formula (VIaf) or (VIaf-a) or (VIaf-b):

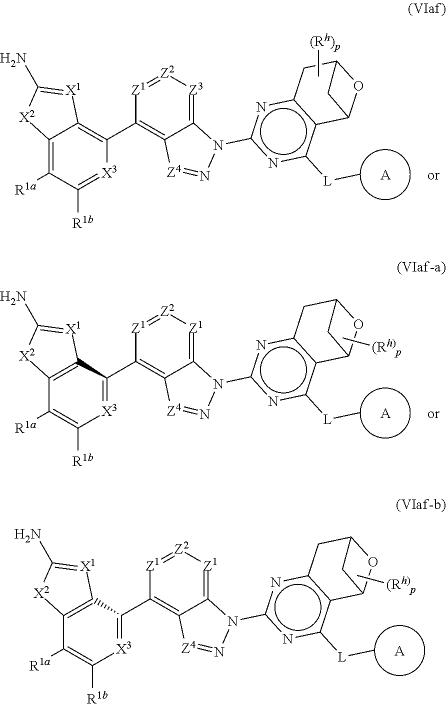

or a pharmaceutically acceptable salt thereof, wherein:
R^{h} is each independently selected from D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂; and
p is selected from 0, 1, 2, 3, and 4, and the remainder of the variables are as described in embodiment (1);
   (I) the compound is of Formula (VIag) or (VIag-a) or (VIag-b):

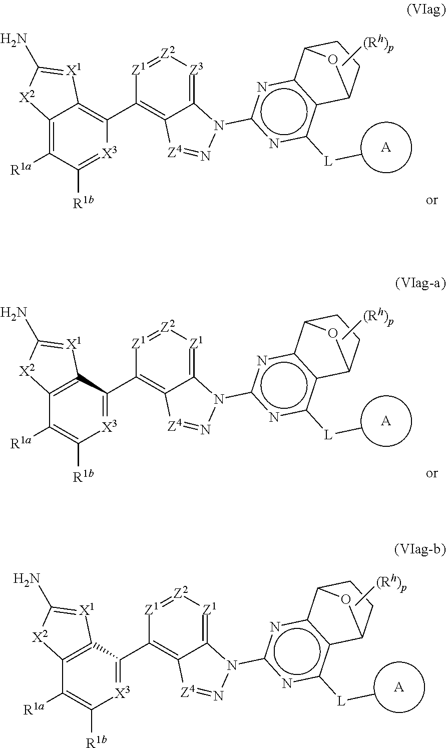

or a pharmaceutically acceptable salt thereof, wherein:
R^{h} is each independently selected from D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂; and
p is selected from 0, 1, 2, 3, and 4, and the remainder of the variables are as described in embodiment (1);
   (J) the compound is of Formula (VIah) or (VIah-a) or (VIah-b):

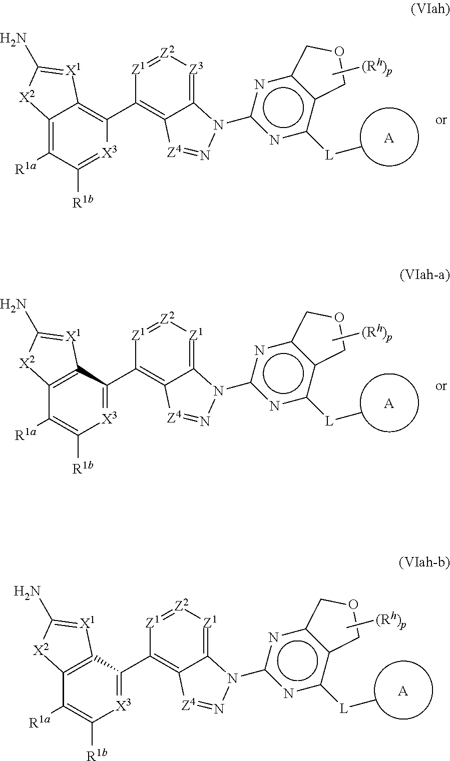

or a pharmaceutically acceptable salt thereof, wherein:
R^{h} is each independently selected from D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂; and
p is selected from 0, 1, 2, 3, and 4, and the remainder of the variables are as described in embodiment (1);
   (K) the compound is of Formula (VIai) or (VIai-a) or (VIai-b):

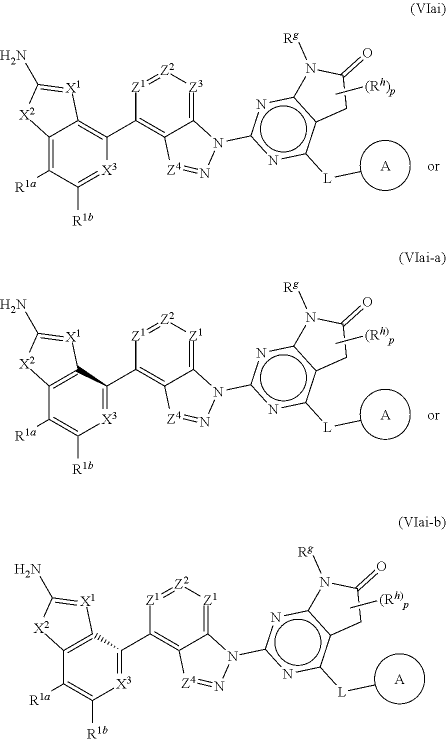

or a pharmaceutically acceptable salt thereof, wherein:
R^{h} is each independently selected from D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂; and
p is selected from 0, 1, 2, 3, and 4, and the remainder of the variables are as described in embodiment (1);
   (L) the compound is of Formula (VIaj) or (VIaj-a) or (VIaj-b):

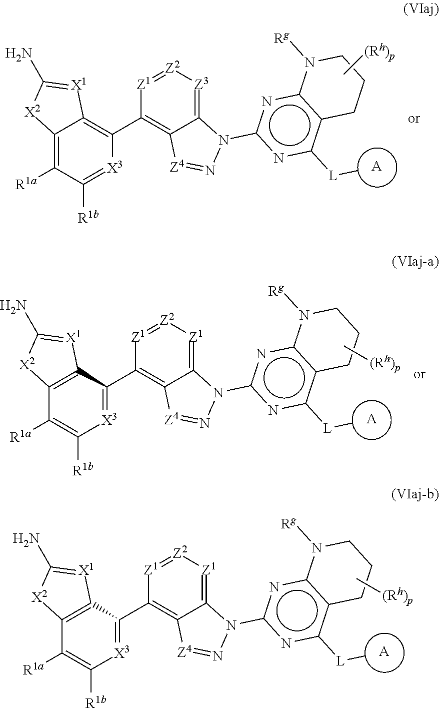

or a pharmaceutically acceptable salt thereof, wherein:
R^{h} is each independently selected from D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂; and
p is selected from 0, 1, 2, 3, and 4, and the remainder of the variables are as described in embodiment (1);
   (M) the compound is of Formula (VIak) or (VIak-a) or (VIak-b):

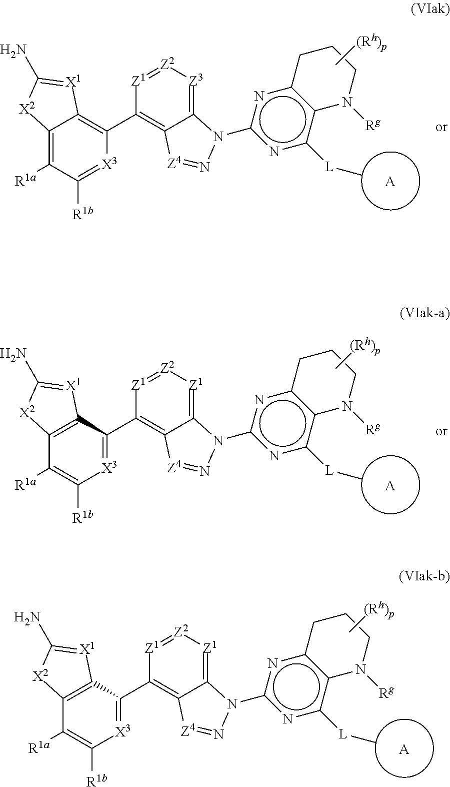

or a pharmaceutically acceptable salt thereof, wherein:
R^{h} is each independently selected from D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂; and
p is selected from 0, 1, 2, 3, and 4, and the remainder of the variables are as described in embodiment (1);
   (N) the compound is of Formula (VIal) or (VIal-a) or (VIal-b):

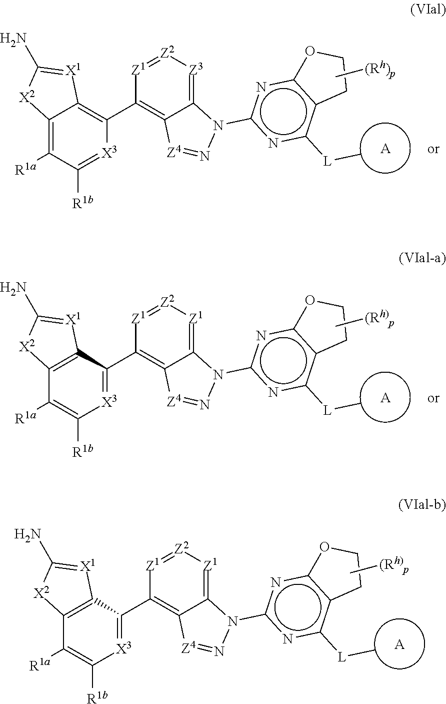

or a pharmaceutically acceptable salt thereof, wherein:
R^{h} is each independently selected from D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂; and
p is selected from 0, 1, 2, 3, and 4, and the remainder of the variables are as described in embodiment (1);
   (O) the compound is of Formula (VIam) or (VIam-a) or (VIam-b):

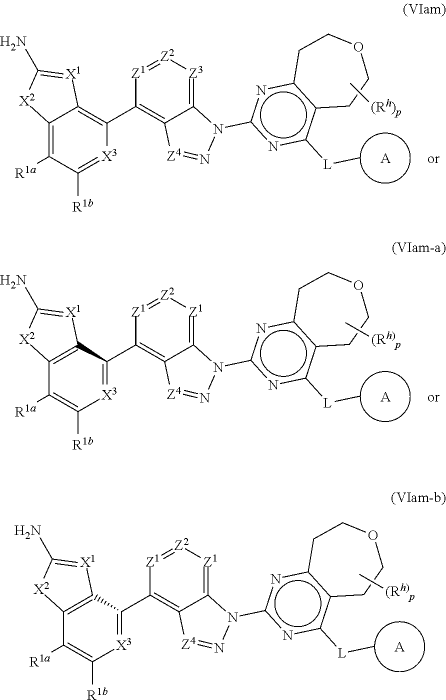

or a pharmaceutically acceptable salt thereof, wherein:
R^{h} is each independently selected from D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂; and
p is selected from 0, 1, 2, 3, and 4, and the remainder of the variables are as described in embodiment (1);
   (P) the compound is of Formula (VIan) or (VIan-a) or (VIan-b):

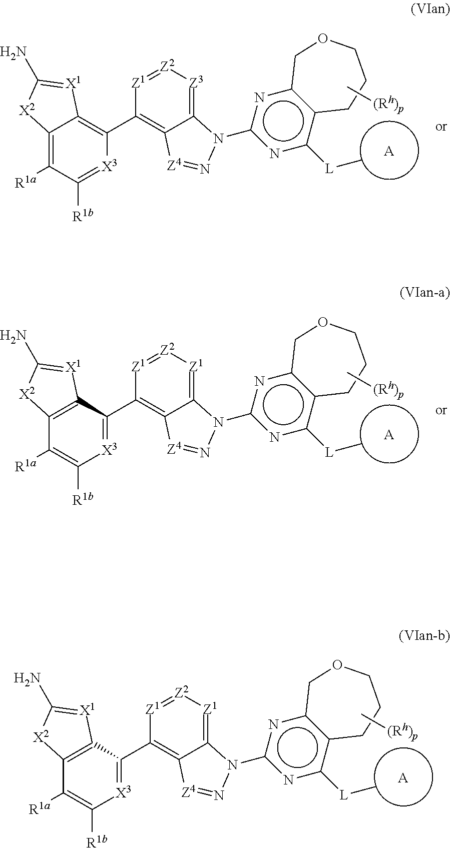

or a pharmaceutically acceptable salt thereof, wherein:
R^{h} is each independently selected from D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂; and
p is selected from 0, 1, 2, 3, and 4, and the remainder of the variables are as described in embodiment (1);
   (Q) the compound is of Formula (VIao) or (VIao-a) or (VIao-b):

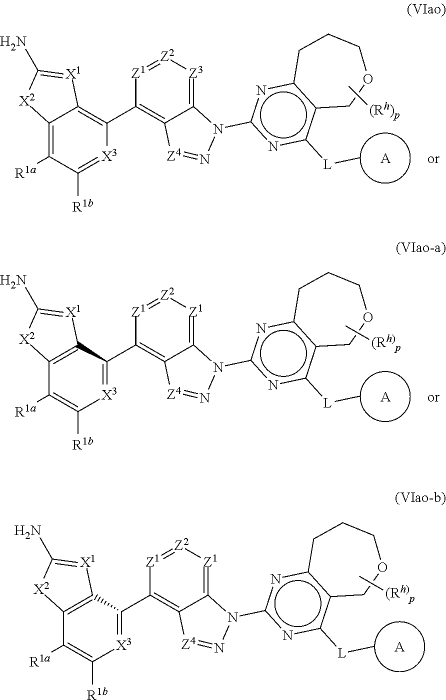

or a pharmaceutically acceptable salt thereof, wherein:
R^{h} is each independently selected from D, halo, ═O, OR^{g}, N(R^{g})₂, and C₁-C₄ alkyl, wherein the and N(R^{g})₂; and
p is selected from 0, 1, 2, 3, and 4, and the remainder of the variables are as described in embodiment (1).

In addition to embodiments (1) through (25) in the preceding paragraphs, the compounds of Formula (I), (I-a), (I-b), (VIa), (VIa-a), (VIa-b), (VIb), (VIb-a), (VIb-b), (VIc), (VIc-a), (VIc-b), (VId), (VId-a), (VId-b), (VIe), (VIe-a), (VIe-b), (VIf), (VIf-a), (VIf-b), (VIg), (VIg-a), (VIg-b), (VIh), (VIh-a), (VIh-b), (VIi), (VIi-a), (VIi-b), (VIj), (VIj-a), (VIj-b), (VIk), (VIk-a), (VIk-b), (VII), (VII-a), (VII-b), (VIm), (VIm-a), (VIm-b), (VIn), (VIn-a), (VIn-b), (VIo), (VIo-a), (VIo-b), (VIp), (VIp-a), (VIp-b), (VIq), (VIq-a), (VIq-b), (VIr), (VIr-a), (VIr-b), (VIs), (VIs-a), (VIs-b), (VIt), (VIt-a), (VIt-b), (VIu), (VIu-a), (VIu-b), (VIv), (VIv-a), (VIv-b), (VIw), (VIw-a), (VIw-b), (VIx), (VIx-a), (VIx-b), (VIy), (VIy-a), (VIy-b), (VIz), (VIz-a), (VIz-b), (VIaa), (VIaa-a), (VIaa-b), (VIab), (VIab-a), (VIab-b), (VIac), (VIac-a), (VIac-b), (VIad), (VIad-a), (VIad-b), (VIae), (VIae-a), (VIae-b), (VIaf), (VIaf-a), (VIaf-b), (VIag), (VIag-a), (VIag-b), (VIah), (VIah-a), (VIah-b), (VIai), (VIai-a), (VIai-b), (VIaj), (VIaj-a), (VIaj-b), (VIak), (VIak-a), (VIak-b), (VIal), (VIal-a), (VIal-b), (VIam), (VIam-a), (VIam-b), (VIan), (Vlan-a), (Vlan-b), (VIao), (VIao-a), or (VIao-b), or pharmaceutically acceptable salts thereof, include those in which:

### (26) X¹ is N or C—CN;

X² is S or Se;
X³ is N or CR^{1c};
Z¹ is CR^{a}, Z² is CR^{b}; and Z³ is CR^{c} or N;
Z⁴ is CR^{d} or N;
L is selected from a bond and O(CR^{La}R^{Lb})ₘ₂—;
R^{La} and R^{Lb} are each independently selected from H, D, and C₁-C₄ alkyl, wherein the alkyl is optionally substituted with 1 to 4 groups independently selected from D and halo;
m2 is selected from 0, 1, and 2;
Ring A is a C₃-C₈ cycloalkyl or 3 to 10 membered heterocyclyl ring, wherein the cycloalkyl or heterocyclyl ring are optionally substituted with 1 to 4 groups independently selected from D, halo, ═O, OR^{g}, N(R^{g})₂, and C₁-C₄ alkyl, and wherein the alkyl is optionally substituted with 1 to 4 groups independently selected from D and halo, wherein the heterocyclyl ring comprises 1 or 2 heteroatoms selected from N, NR^{g}, and O;
R^{1a} and R^{1c} are each independently selected from H, halo, and C₁-C₄ alkyl and R^{1b} is H;
R^{a}, R^{b}, R^{c} and R^{d} are each independently selected from H, D, halo, OR^{g}, N(R^{g})₂, and C₁-C₄ alkyl, wherein the alkyl is optionally substituted with 1 to 4 groups independently selected from D, halo, ═O, OR^{g}, and N(R^{g})₂; or
R^{a} and R^{b}, together with the carbon atoms to which they are attached, form a cyclopentyl, cyclohexyl, or a 5 to 6 membered heterocyclyl, wherein the cyclopentyl, cyclohexyl, or 5 to 6 membered heterocyclyl are optionally substituted with 1 to 3 groups independently selected from D, halo, C₁-C₄ alkyl, and C₁-C₄ alkoxy, wherein the alkyl and alkoxy are optionally substituted with 1 to 3 groups independently selected from D, halo, and OR^{g}, and the heterocyclyl comprises 1 or 2 heteroatoms selected from NR^{g} and O; or
R^{b} and R^{c}, together with the carbon atoms to which they are attached, form a cyclopentyl, cyclohexyl, or a 5 to 6 membered heterocyclyl, wherein the cyclopentyl, cyclohexyl, or 5 to 6 membered heterocyclyl are optionally substituted with 1 to 3 groups independently selected from D, halo, C₁-C₄ alkyl, and C₁-C₄ alkoxy, wherein the alkyl and alkoxy are optionally substituted with 1 to 3 groups independently selected from D, halo, and ORS, and the heterocyclyl comprises 1 or 2 heteroatoms selected from NR^{g} and O; and
R^{g} is each independently selected from H and C₁-C₄ alkyl, wherein the alkyl is optionally substituted with 1 to 4 groups independently selected from D and halo, and the remainder of the variables are as described in embodiments (1) and (11) through (25).

In addition to embodiments (1) through (26) in the preceding paragraphs, the compounds of Formula (I), (I-a), (I-b), (II), (II-a), (II-b), (IVa), (IVa-a), (IVa-b), (IVb), (IVb-a), (IVb-b), (IVc), (IVc-a), (IVc-b), (IVd), (IVd-a), (IVd-b), (IVe), (IVe-a), (IVe-b), (IVf), (IVf-a), (IVf-b), (IVg), (IVg-a), (IVg-b), (IVh), (IVh-a), (IVh-b), (IVi), (IVi-a), (IVi-b), (IVj), (IVj-a), (IVj-b), (IVk), (IVk-a), (IVk-b), (IVl), (IVl-a), (IVl-b), (IVm), (IVm-a), (IVm-b), (IVn), (IVn-a), (IVn-b), (Va), (Va-a), (Va-b), (Vb), (Vb-a), (Vb-b), (Vc), (Vc-a), (Vc-b), (Vd), (Vd-a), (Vd-b), (Ve), (Ve-a), (Ve-b), (Vf), (Vf-a), (Vf-b), (Vg), (Vg-a), (Vg-b), (Vh), (Vh-a), (Vh-b), (VIa), (VIa-a), (VIa-b), (VIb), (VIb-a), (VIb-b), (VIc), (VIc-a), (VIc-b), (VId), (VId-a), (VId-b), (VIe), (VIe-a), (VIe-b), (VIf), (VIf-a), (VIf-b), (VIg), (VIg-a), (VIg-b), (VIh), (VIh-a), (VIh-b), (VIi), (VIi-a), (VIi-b), (VIj), (VIj-a), (VIj-b), (VIk), (VIk-a), (VIk-b), (VII), (VII-a), (VII-b), (VIm), (VIm-a), (VIm-b), (VIn), (VIn-a), (VIn-b), (VIo), (VIo-a), (VIo-b), (VIp), (VIp-a), (VIp-b), (VIq), (VIq-a), (VIq-b), (VIr), (VIr-a), (VIr-b), (VIs), (VIs-a), (VIs-b), (VIt), (VIt-a), (VIt-b), (VIu), (VIu-a), (VIu-b), (VIv), (VIv-a), (VIv-b), (VIw), (VIw-a), (VIw-b), (VIx), (VIx-a), (VIx-b), (VIy), (VIy-a), (VIy-b), (VIz), (VIz-a), (VIz-b), (VIaa), (VIaa-a), (VIaa-b), (VIab), (VIab-a), (VIab-b), (VIac), (VIac-a), (VIac-b), (VIad), (VIad-a), (VIad-b), (VIae), (VIae-a), (VIae-b), (VIaf), (VIaf-a), (VIaf-b), (VIag), (VIag-a), (VIag-b), (VIah), (VIah-a), (VIah-b), (VIai), (VIai-a), (VIai-b), (VIaj), (VIaj-a), (VIaj-b), (VIak), (VIak-a), (VIak-b), (VIal), (VIal-a), (VIal-b), (VIam), (VIam-a), (VIam-b), (VIan), (VIan-a), (VIan-b), (VIao), (VIao-a), or (VIao-b), or pharmaceutically acceptable salts thereof, include those in which:

(27) (A) L is a bond, and the remainder of the variables are as described in embodiments (1) through (3), (5) through (7), and (9) through (26);
(B) L is —(CR^{La}R^{Lb})(CR^{La}R^{Lb})—, wherein R^{La} and R^{Lb} are each independently selected from H and C₁-C₃ alkyl, wherein the alkyl is optionally substituted with 1 to 3 groups independently selected from D, F, Cl, and Br; for example (C) R^{La} and R^{Lb} are each independently selected from H and methyl, and the remainder of the variables are as described in embodiments (1) through (3), (5) through (7), and (9) through (26);
(D) L is —CR^{La}R^{Lb}—, wherein R^{La} and R^{Lb} are each independently selected from H and C₁-C₃ alkyl, wherein the alkyl is optionally substituted with 1 to 3 groups independently selected from D, F, Cl, and Br; for example (E) R^{La} and R^{Lb} are each independently selected from H and methyl, and the remainder of the variables are as described in embodiments (1) through (3), (5) through (7), and (9) through (26);
(F) L is —(CR^{La}R^{Lb})O(CR^{La}R^{Lb})—, wherein R^{La} and R^{Lb} are each independently selected from H and C₁-C₃ alkyl, wherein the alkyl is optionally substituted with 1 to 3 groups independently selected from D, F, Cl, and Br; for example (G) R^{La} and R^{Lb} are each independently selected from H and methyl, and the remainder of the variables are as described in embodiments (1) through (3), (5) through (7), and (9) through (26);
(H) L is —O(CR^{La}R^{Lb})—, wherein R^{La} and R^{Lb} are each independently selected from H and C₁-C₃ alkyl, wherein the alkyl is optionally substituted with 1 to 3 groups independently selected from D, F, Cl, and Br; for example (I) R^{La} and R^{Lb} are each independently selected from H and methyl, and the remainder of the variables are as described in embodiments (1) through (3), (5) through (7), and (9) through (26);
(J) L is —O(CR^{La}R^{Lb})₂—, wherein R^{La} and R^{Lb} are each independently selected from H and C₁-C₃ alkyl, wherein the alkyl is optionally substituted with 1 to 3 groups independently selected from D, F, Cl, and Br; for example (K) R^{La} and R^{Lb} are each independently selected from H and methyl, and the remainder of the variables are as described in embodiments (1) through (3), (5) through (7), and (9) through (26);
(L) L is —O(CR^{La}R^{Lb})₃—, wherein R^{La} and R^{Lb} are each independently selected from H and C₁-C₃ alkyl, wherein the alkyl is optionally substituted with 1 to 3 groups independently selected from D, F, Cl, and Br; for example (M) R^{La} and R^{Lb} are each independently selected from H and methyl, and the remainder of the variables are as described in embodiments (1) through (3), (5) through (7), and (9) through (26);
(N) L is —O—, and the remainder of the variables are as described in embodiments (1) through (3), (5) through (7), and (9) through (26);
(O) L is —(CR^{La}R^{Lb})NR^{L}(CR^{La}R^{Lb})—, wherein R^{La} and R^{Lb} are each independently selected from H and C₁-C₃ alkyl, wherein the alkyl is optionally substituted with 1 to 3 groups independently selected from D, F, Cl, and Br; for example (P) R^{La} and R^{Lb} are each independently selected from H and methyl; and wherein R^{L} is H or methyl, and the remainder of the variables are as described in embodiments (1) through (3), (5) through (7), and (9) through (26);
(Q) L is —NR^{L}(CR^{La}R^{Lb})—, wherein R^{La} and R^{Lb} are each independently selected from H and C₁-C₃ alkyl, wherein the alkyl is optionally substituted with 1 to 3 groups independently selected from D, F, Cl, and Br; for example (R) R^{La} and R^{Lb} are each independently selected from H and methyl; and wherein R^{L} is H or methyl, and the remainder of the variables are as described in embodiments (1) through (3), (5) through (7), and (9) through (26);
(S) L is —NR^{L}—, wherein R^{L} is H or methyl, and the remainder of the variables are as described in embodiments (1) through (3), (5) through (7), and (9) through (26).

In addition to embodiments (1) through (27) in the preceding paragraphs, the compounds of Formula (I), (I-a), (I-b), (II), (II-a), (II-b), (IVa), (IVa-a), (IVa-b), (IVb), (IVb-a), (IVb-b), (IVc), (IVc-a), (IVc-b), (IVd), (IVd-a), (IVd-b), (IVe), (IVe-a), (IVe-b), (IVf), (IVf-a), (IVf-b), (IVg), (IVg-a), (IVg-b), (IVh), (IVh-a), (IVh-b), (IVi), (IVi-a), (IVi-b), (IVj), (IVj-a), (IVj-b), (IVk), (IVk-a), (IVk-b), (IVl), (IVl-a), (IVl-b), (IVm), (IVm-a), (IVm-b), (IVn), (IVn-a), (IVn-b), (Va), (Va-a), (Va-b), (Vb), (Vb-a), (Vb-b), (Vc), (Vc-a), (Vc-b), (Vd), (Vd-a), (Vd-b), (Ve), (Ve-a), (Ve-b), (Vf), (Vf-a), (Vf-b), (Vg), (Vg-a), (Vg-b), (Vh), (Vh-a), (Vh-b), (VIa), (VIa-a), (VIa-b), (VIb), (VIb-a), (VIb-b), (VIc), (VIc-a), (VIc-b), (VId), (VId-a), (VId-b), (VIe), (VIe-a), (VIe-b), (VIf), (VIf-a), (VIf-b), (VIg), (VIg-a), (VIg-b), (VIh), (VIh-a), (VIh-b), (VIi), (VIi-a), (VIi-b), (VIj), (VIj-a), (VIj-b), (VIk), (VIk-a), (VIk-b), (VII), (VII-a), (VII-b), (VIm), (VIm-a), (VIm-b), (VIn), (VIn-a), (VIn-b), (VIo), (VIo-a), (VIo-b), (VIp), (VIp-a), (VIp-b), (VIq), (VIq-a), (VIq-b), (VIr), (VIr-a), (VIr-b), (VIs), (VIs-a), (VIs-b), (VIt), (VIt-a), (VIt-b), (VIu), (VIu-a), (VIu-b), (VIv), (VIv-a), (VIv-b), (VIw), (VIw-a), (VIw-b), (VIx), (VIx-a), (VIx-b), (VIy), (VIy-a), (VIy-b), (VIz), (VIz-a), (VIz-b), (VIaa), (VIaa-a), (VIaa-b), (VIab), (VIab-a), (VIab-b), (VIac), (VIac-a), (VIac-b), (VIad), (VIad-a), (VIad-b), (VIae), (VIae-a), (VIae-b), (VIaf), (VIaf-a), (VIaf-b), (VIag), (VIag-a), (VIag-b), (VIah), (VIah-a), (VIah-b), (VIai), (VIai-a), (VIai-b), (VIaj), (VIaj-a), (VIaj-b), (VIak), (VIak-a), (VIak-b), (VIal), (VIal-a), (VIal-b), (VIam), (VIam-a), (VIam-b), (VIan), (VIan-a), (VIan-b), (VIao), (VIao-a), or (VIao-b), or pharmaceutically acceptable salts thereof, include those in which

(28) (A) Ring A is a C₃-C₈ cycloalkyl optionally substituted with 1 to 4 groups independently selected from D, halo, ═O, OR^{g}, NR^{g}R^{g}, C(O)NR^{g}R^{g}, —CN, C₁-C₄ alkyl, and C₂-C₄ alkenyl, wherein the alkyl and alkenyl are optionally substituted with 1 to 4 groups independently selected from D, halo, ═O, OR^{g}, NR^{g}R^{g}, C(O)NR^{g}R^{g}, CN, and 3 to 8 membered heterocyclyl ring, wherein the heterocyclyl ring is optionally substituted with 1 to 4 groups independently selected from C₁-C₄ alkyl and comprises 1 or 2 heteroatoms selected from N, NH, O, and S, and the remainder of the variables are as described in embodiments (1) through (3), (5) through (7), and (9) through (27);
(B) Ring A is a C₃-C₆ cycloalkyl optionally substituted with 1 to 3 groups independently selected from D, halo, OR^{g}, NR^{g}R^{g}, —CN, and C₁-C₄ alkyl, wherein the alkyl is optionally substituted with 1 to 3 groups independently selected from D, halo, OR^{g}, NR^{g}R^{g}, CN, and 4 to 6 membered heterocyclyl ring, wherein the heterocyclyl ring is optionally substituted with 1 to 3 groups independently selected from C₁-C₄ alkyl and comprises 1 or 2 heteroatoms selected from N, NH, and O, and the remainder of the variables are as described in embodiments (1) through (3), (5) through (7), and (9) through (27);
(C) Ring A is a C₃-C₆ cycloalkyl optionally substituted with 1 to 3 groups independently selected from D, halo, OR^{g}, NR^{g}R^{g}, and C₁-C₄ alkyl, wherein the alkyl is optionally substituted with 1 to 3 groups independently selected from D, halo, OR^{g}, and NR^{g}R^{g}, and the remainder of the variables are as described in embodiments (1) through (3), (5) through (7), and (9) through (27);
(D) Ring A is phenyl optionally substituted with 1 to 3 groups independently selected from D, halo, OH, NH₂, NHCH₃, N(CH₃)₂, CN, C₁-C₄ alkyl, and C₁-C₄ alkoxy, wherein the alkyl and alkoxy are optionally substituted with 1 to 3 groups independently selected from D, halo, OH, NH₂, NHCH₃, N(CH₃)₂, and CN, and the remainder of the variables are as described in embodiments (1) through (3), (5) through (7), and (9) through (27);
(E) Ring A is phenyl, and the remainder of the variables are as described in embodiments (1) through (3), (5) through (7), and (9) through (27);
(F) Ring A is a 3 to 10 membered heterocyclyl ring optionally substituted with 1 to 4 groups independently selected from D, halo, ═O, OR^{g}, NR^{g}R^{g}, C(O)NR^{g}R^{g}, —CN, C₁-C₄ alkyl, and C₂-C₄ alkenyl, wherein the alkyl and alkenyl are optionally substituted with 1 to 4 groups independently selected from D, halo, ═O, OR^{g}, NR^{g}R^{g}, C(O)NR^{g}R^{g}, and CN, wherein the heterocyclyl comprises 1 or 2 heteroatoms selected from N, NH, O, and S, and the remainder of the variables are as described in embodiments (1) through (3), (5) through (7), and (9) through (27);
(G) Ring A is a 4 to 9 membered heterocyclyl ring optionally substituted with 1 to 3 groups independently selected from D, halo, ═O, OR^{g}, NR^{g}R^{g}, —CN, and C₁-C₄ alkyl, wherein the alkyl is optionally substituted with 1 to 3 groups independently selected from D, halo, ═O, OR^{g}, NR^{g}R^{g}, and CN, wherein the heterocyclyl comprises 1 or 2 heteroatoms selected from N, NH, and O, and the remainder of the variables are as described in embodiments (1) through (3), (5) through (7), and (9) through (27);
(H) Ring A is a 4 to 9 membered heterocyclyl ring optionally substituted with 1 to 3 groups independently selected from D, halo, OR^{g}, NR^{g}R^{g}, and C₁-C₄ alkyl, wherein the alkyl is optionally substituted with 1 to 3 groups independently selected from D, halo, OR^{g}, and NR^{g}R^{g}, wherein the heterocyclyl comprises 1 or 2 heteroatoms selected from N, NH, and O, and the remainder of the variables are as described in embodiments (1) through (3), (5) through (7), and (9) through (27);
(I) Ring A is a 5 to 10 membered heteroaryl ring optionally substituted with 1 to 4 groups independently selected from D, halo, ═O, OR^{g}, NR^{g}R^{g}, C(O)NR^{g}R^{g}, —CN, C₁-C₄ alkyl, and C₂-C₄ alkenyl, wherein the alkyl and alkenyl are optionally substituted with 1 to 4 groups independently selected from D, halo, ═O, OR^{g}, NR^{g}R^{g}, C(O)NR^{g}R^{g}, CN, and 3 to 8 membered heterocyclyl ring, wherein the heterocyclyl ring is optionally substituted with 1 to 4 groups independently selected from C₁-C₄ alkyl, wherein the heteroaryl ring comprises 1 or 2 heteroatoms selected from N, NH, O, and S, and the remainder of the variables are as described in embodiments (1) through (3), (5) through (7), and (9) through (27);
(J) Ring A is a 5 to 10 membered heteroaryl ring optionally substituted with 1 to 3 groups independently selected from D, halo, ═O, OR^{g}, NR^{g}R^{g}, C(O)NR^{g}R^{g}, —CN, and C₁-C₄ alkyl, wherein the alkyl is optionally substituted with 1 to 3 groups independently selected from D, halo, ═O, OR^{g}, NR^{g}R^{g}, C(O)NR^{g}R^{g}, and CN, wherein the heteroaryl ring comprises 1 or 2 heteroatoms selected from N, NH, O, and S, and the remainder of the variables are as described in embodiments (1) through (3), (5) through (7), and (9) through (27);
(K) Ring A is a 4 to 9 membered heterocyclyl ring optionally substituted with 1 to 3 groups independently selected from D, halo, OR^{g}, NR^{g}R^{g}, and C₁-C₄ alkyl, wherein the alkyl is optionally substituted with 1 to 3 groups independently selected from D, halo, OR^{g}, and NR^{g}R^{g}, wherein the heterocyclyl comprises 1 or 2 heteroatoms selected from N, NH, and O, and the remainder of the variables are as described in embodiments (1) through (3), (5) through (7), and (9) through (27).

In addition to embodiments (1) through (28) in the preceding paragraphs, the compounds of Formula (I), (I-a), (I-b), (II), (II-a), (II-b), (III), (III-a), (III-b), (IVa), (IVa-a), (IVa-b), (IVb), (IVb-a), (IVb-b), (IVc), (IVc-a), (IVc-b), (IVd), (IVd-a), (IVd-b), (IVe), (IVe-a), (IVe-b), (IVf), (IVf-a), (IVf-b), (IVg), (IVg-a), (IVg-b), (IVh), (IVh-a), (IVh-b), (IVi), (IVi-a), (IVi-b), (IVj), (IVj-a), (IVj-b), (IVk), (IVk-a), (IVk-b), (IVl), (IVl-a), (IVl-b), (IVm), (IVm-a), (IVm-b), (IVn), (IVn-a), (IVn-b), (Va), (Va-a), (Va-b), (Vb), (Vb-a), (Vb-b), (Vc), (Vc-a), (Vc-b), (Vd), (Vd-a), (Vd-b), (Ve), (Ve-a), (Ve-b), (Vf), (Vf-a), (Vf-b), (Vg), (Vg-a), (Vg-b), (Vh), (Vh-a), (Vh-b), (VIa), (VIa-a), (VIa-b), (VIb), (VIb-a), (VIb-b), (VIc), (VIc-a), (VIc-b), (VId), (VId-a), (VId-b), (VIe), (VIe-a), (VIe-b), (VIf), (VIf-a), (VIf-b), (VIg), (VIg-a), (VIg-b), (VIh), (VIh-a), (VIh-b), (VIi), (VIi-a), (VIi-b), (VIj), (VIj-a), (VIj-b), (VIk), (VIk-a), (VIk-b), (VIl), (VIl-a), (VIl-b), (VIm), (VIm-a), (VIm-b), (VIn), (VIn-a), (VIn-b), (VIo), (VIo-a), (VIo-b), (VIp), (VIp-a), (VIp-b), (VIq), (VIq-a), (VIq-b), (VIr), (VIr-a), (VIr-b), (VIs), (VIs-a), (VIs-b), (VIt), (VIt-a), (VIt-b), (VIu), (VIu-a), (VIu-b), (VIv), (VIv-a), (VIv-b), (VIw), (VIw-a), (VIw-b), (VIx), (VIx-a), (VIx-b), (VIy), (VIy-a), (VIy-b), (VIz), (VIz-a), (VIz-b), (VIaa), (VIaa-a), (VIaa-b), (VIab), (VIab-a), (VIab-b), (VIac), (VIac-a), (VIac-b), (VIad), (VIad-a), (VIad-b), (VIae), (VIae-a), (VIae-b), (VIaf), (VIaf-a), (VIaf-b), (VIag), (VIag-a), (VIag-b), (VIah), (VIah-a), (VIah-b), (VIai), (VIai-a), (VIai-b), (VIaj), (VIaj-a), (VIaj-b), (VIak), (VIak-a), (VIak-b), (VIal), (VIal-a), (VIal-b), (VIam), (VIam-a), (VIam-b), (VIan), (VIan-a), (VIan-b), (VIao), (VIao-a), or (VIao-b), or pharmaceutically acceptable salts thereof, include those in which:

(29) (A) R^{1a}, R^{1b}, and R^{1c} are each H, and the remainder of the variables are as described in embodiments (1) through (28);
(B) R^{1a} is halo and R^{1b} and R^{1c} are each H, and the remainder of the variables are as described in embodiments (1) through (28);
(C) R^{1a} is F or Cl, and R^{1b} and R^{1c} are each H, and the remainder of the variables are as described in embodiments (1) through (28);
(D) R^{1a} and R^{1b} are each H and R^{1c} is F, and the remainder of the variables are as described in embodiments (1) through (28);
(E) R^{1a} and R^{1c} are each F and R^{1b} is H, and the remainder of the variables are as described in embodiments (1) through (28).

In addition to embodiments (1) through (29) in the preceding paragraphs, the compounds of Formula (I), (I-a), (I-b), (II), (II-a), (II-b), (III), (III-a), (III-b), (IVa), (IVa-a), (IVa-b), (IVb), (IVb-a), (IVb-b), (IVc), (IVc-a), (IVc-b), (IVd), (IVd-a), (IVd-b), (IVe), (IVe-a), (IVe-b), (IVf), (IVf-a), (IVf-b), (IVg), (IVg-a), (IVg-b), (IVh), (IVh-a), (IVh-b), (IVi), (IVi-a), (IVi-b), (IVj), (IVj-a), (IVj-b), (IVk), (IVk-a), (IVk-b), (IVl), (IVl-a), (IVl-b), (IVm), (IVm-a), (IVm-b), (IVn), (IVn-a), (IVn-b), (Va), (Va-a), (Va-b), (Vb), (Vb-a), (Vb-b), (Vc), (Vc-a), (Vc-b), (Vd), (Vd-a), (Vd-b), (Ve), (Ve-a), (Ve-b), (Vf), (Vf-a), (Vf-b), (Vg), (Vg-a), (Vg-b), (Vh), (Vh-a), (Vh-b), (VIa), (VIa-a), (VIa-b), (VIb), (VIb-a), (VIb-b), (VIc), (VIc-a), (VIc-b), (VId), (VId-a), (VId-b), (VIe), (VIe-a), (VIe-b), (VIf), (VIf-a), (VIf-b), (VIg), (VIg-a), (VIg-b), (VIh), (VIh-a), (VIh-b), (VIi), (VIi-a), (VIi-b), (VIj), (VIj-a), (VIj-b), (VIK), (VIk-a), (VIk-b), (VII), (VII-a), (VII-b), (VIm), (VIm-a), (VIm-b), (VIn), (VIn-a), (VIn-b), (VIo), (VIo-a), (VIo-b), (VIp), (VIp-a), (VIp-b), (VIq), (VIq-a), (VIq-b), (VIr), (VIr-a), (VIr-b), (VIs), (VIs-a), (VIs-b), (VIt), (VIt-a), (VIt-b), (VIu), (VIu-a), (VIu-b), (VIv), (VIv-a), (VIv-b), (VIw), (VIw-a), (VIw-b), (VIx), (VIx-a), (VIx-b), (VIy), (VIy-a), (VIy-b), (VIz), (VIz-a), (VIz-b), (VIaa), (VIaa-a), (VIaa-b), (VIab), (VIab-a), (VIab-b), (VIac), (VIac-a), (VIac-b), (VIad), (VIad-a), (VIad-b), (VIae), (VIae-a), (VIae-b), (VIaf), (VIaf-a), (VIaf-b), (VIag), (VIag-a), (VIag-b), (VIah), (VIah-a), (VIah-b), (VIai), (VIai-a), (VIai-b), (VIaj), (VIaj-a), (VIaj-b), (VIak), (VIak-a), (VIak-b), (VIal), (VIal-a), (VIal-b), (VIam), (VIam-a), (VIam-b), (VIan), (VIan-a), (VIan-b), (VIao), (VIao-a), or (VIao-b), or pharmaceutically acceptable salts thereof, include those in which:

(30) (A) R^{a}, R^{b}, R^{c} and R^{d} are each independently selected from H, D, halo, OR^{g}, and C₁-C₄ alkyl, wherein the alkyl is optionally substituted with 1 to 3 groups independently selected from D, halo, and OR^{g}; for example, (B) R^{a} is selected from H, F, Cl, Me, CF3, and OMe, R^{d} is selected from H, F, Cl, and Me, and R^{b} and R^{e} are both H, and the remainder of the variables are as described in embodiments (1) through (29).

In addition to embodiments (1) through (30) in the preceding paragraphs, the compounds of Formula (I), (I-a), (I-b), (II), (II-a), (II-b), (III), (III-a), (III-b), (IVa), (IVa-a), (IVa-b), (IVb), (IVb-a), (IVb-b), (IVc), (IVc-a), (IVc-b), (IVd), (IVd-a), (IVd-b), (IVe), (IVe-a), (IVe-b), (IVf), (IVf-a), (IVf-b), (IVg), (IVg-a), (IVg-b), (IVh), (IVh-a), (IVh-b), (IVi), (IVi-a), (IVi-b), (IVj), (IVj-a), (IVj-b), (IVk), (IVk-a), (IVk-b), (IVl), (IVl-a), (IVl-b), (IVm), (IVm-a), (IVm-b), (IVn), (IVn-a), (IVn-b), (Va), (Va-a), (Va-b), (Vb), (Vb-a), (Vb-b), (Vc), (Vc-a), (Vc-b), (Vd), (Vd-a), (Vd-b), (Ve), (Ve-a), (Ve-b), (Vf), (Vf-a), (Vf-b), (Vg), (Vg-a), (Vg-b), (Vh), (Vh-a), (Vh-b), (VIa), (VIa-a), (VIa-b), (VIb), (VIb-a), (VIb-b), (VIc), (VIc-a), (VIc-b), (VId), (VId-a), (VId-b), (VIe), (VIe-a), (VIe-b), (VIf), (VIf-a), (VIf-b), (VIg), (VIg-a), (VIg-b), (VIh), (VIh-a), (VIh-b), (VIi), (VIi-a), (VIi-b), (VIj), (VIj-a), (VIj-b), (VIk), (VIk-a), (VIk-b), (VII), (VII-a), (VII-b), (VIm), (VIm-a), (VIm-b), (VIn), (VIn-a), (VIn-b), (VIo), (VIo-a), (VIo-b), (VIp), (VIp-a), (VIp-b), (VIq), (VIq-a), (VIq-b), (VIr), (VIr-a), (VIr-b), (VIs), (VIs-a), (VIs-b), (VIt), (VIt-a), (VIt-b), (VIu), (VIu-a), (VIu-b), (VIv), (VIv-a), (VIv-b), (VIw), (VIw-a), (VIw-b), (VIx), (VIx-a), (VIx-b), (VIy), (VIy-a), (VIy-b), (VIz), (VIz-a), (VIz-b), (VIaa), (VIaa-a), (VIaa-b), (VIab), (VIab-a), (VIab-b), (VIac), (VIac-a), (VIac-b), (Vlad), (VIad-a), (VIad-b), (VIae), (VIae-a), (VIae-b), (VIaf), (VIaf-a), (VIaf-b), (VIag), (VIag-a), (VIag-b), (VIah), (VIah-a), (VIah-b), (VIai), (VIai-a), (VIai-b), (VIaj), (VIaj-a), (VIaj-b), (VIak), (VIak-a), (VIak-b), (VIal), (VIal-a), (VIal-b), (VIam), (VIam-a), (VIam-b), (VIan), (VIan-a), (VIan-b), (VIao), (VIao-a), or (VIao-b), or pharmaceutically acceptable salts thereof, include those in which:

(31) R^{g} is each independently selected from H, methyl, and ethyl, and the remainder of the variables are as described in embodiments (1) through (30).

In addition to embodiments (1) through (31) in the preceding paragraphs, the compounds of Formula (I), (I-a), or (I-b), or pharmaceutically acceptable salts thereof, include those in which:

### (32) X¹ is C—CN;

X² is S or Se;
X³ is N or CR^{1c};
Z¹ is CR^{a}, Z² is CR^{b} or N; and Z³ is CR^{c};
Z⁴ is CR^{d} or N;
Y¹ is N;
Y² is N;
L is selected from a bond, —(CR^{La}R^{Lb})—, and —(CR^{La}R^{Lb})ₘ₁O(CR^{La}R^{Lb})ₘ₂—;
R^{La} and R^{Lb} are each independently selected from H and C₁-C₄ alkyl, wherein the alkyl is optionally substituted with 1 to 3 groups independently selected from D and halo;
m1 and m2 are each independently selected from 0 and 1;
Ring A is a C₃-C₈ cycloalkyl or 3 to 10 membered heterocyclyl ring, wherein the cycloalkyl or heterocyclyl ring are optionally substituted with 1 to 3 groups independently selected from D, halo, ═O, OR^{g}, NR^{g}R^{g}, C(O)NR^{g}R^{g}, —CN, C₁-C₄ alkyl, and C₂-C₄ alkenyl, and wherein the alkyl and alkenyl are optionally substituted with 1 to 3 groups independently selected from D, halo, ═O, OR^{g}, NR^{g}R^{g}, C(O)NR^{g}R^{g}, CN, and 3 to 8 membered heterocyclyl ring optionally substituted with 1 to 3 groups independently selected from C₁-C₄ alkyl, wherein the heterocyclyl or heteroaryl ring comprises 1 or 2 heteroatoms selected from N, NR^{g}, O, and S;
Ring B is a C₃-C₁₂ cycloalkyl or 4 to 14 membered heterocyclyl, wherein the cycloalkyl or heterocyclyl are optionally substituted with 1 to 3 groups independently selected from D, halo, ═O, OR^{g}, NR^{g}R^{g}, C(O)NR^{g}R^{g}, —CN, C₁-C₄ alkyl, and C₂-C₄ alkenyl, wherein the alkyl and alkenyl are optionally substituted with 1 to 3 groups independently selected from D, halo, ═O, OR^{g}, NR^{g}R^{g}, C(O)NR^{g}R^{g}, and CN, wherein the heterocyclyl comprises 1 or 2 heteroatoms selected from N, NR^{g}, O, and S; or
R^{1a}, R^{1b}, and R^{1c} are each independently selected from H and halo;
R^{a}, R^{b}, R^{c} and R^{d} are each independently selected from H, halo, OR^{g}, N(R^{g})₂, and C₁-C₄ alkyl, wherein the alkyl is optionally substituted with 1 to 3 groups independently selected from D, halo, ═O, OR^{g}, N(R^{g})₂C(O)NR^{g}R^{g}, 5 to 6 membered heterocyclyl and 5 to 6 membered heteroaryl, and wherein the heteroaryl and heterocyclyl comprises 1-3 heteroatoms selected from NR^{g}, O, and S and are optionally substituted with 1 to 3 groups independently selected from D, halo, ═O, C₁-C₄ alkyl, and C₁-C₄ alkoxy, wherein the alkyl and alkoxy are optionally substituted with 1 to 3 groups independently selected from D, halo, and OR^{g}; or
R^{a} and R^{b}, together with the carbon atoms to which they are attached, form a 5 to 6 membered heterocyclyl optionally substituted with 1 to 2 groups independently selected from D, halo, C₁-C₄ alkyl, and C₁-C₄ alkoxy, wherein the alkyl and alkoxy are optionally substituted with 1 to 3 groups independently selected from D, halo, and OR^{g}, and the heterocyclyl comprises 1 or 2 heteroatoms selected from N, NR^{g}, O, and S; and
R^{g} is each independently selected from H and C₁-C₄ alkyl, wherein the alkyl is optionally substituted with 1 to 3 groups independently selected from D and halo.

In addition to embodiments (1) through (32) in the preceding paragraphs, the compounds of Formula (I), (I-a), or (I-b), or pharmaceutically acceptable salts thereof, include those in which:

(33) R^{a}, R^{b}, R^{c} and R^{d} are each independently selected from H, halo, OR^{g}, N(R^{g})₂, and C₁-C₄ alkyl, wherein the alkyl is optionally substituted with 1 to 3 groups independently selected from halo and OR^{g}, or R^{a} and R^{b}, together with the carbon atoms to which they are attached, form a 5 to 6 membered heterocyclyl optionally substituted with 1 to 2 groups independently selected from D, halo, C₁-C₄ alkyl, and C₁-C₄ alkoxy, wherein the alkyl and alkoxy are optionally substituted with 1 to 3 groups independently selected from D, halo, and ORS, and the heterocyclyl comprises 1 or 2 heteroatoms selected from N, NR^{g}, O, and S.

In addition to embodiments (1) through (33) in the preceding paragraphs, the compounds of Formula (I), (I-a), or (I-b), or pharmaceutically acceptable salts thereof, include those in which:

(34) L is —O—C(CH₃)H—, and Ring A is pyrrolidine optionally substituted with methyl, ethyl, halo, or methoxy, wherein the methyl or ethyl is optionally substituted with 1 to 3 D.

In addition to embodiments (1) through (34) in the preceding paragraphs, the compounds of Formula (I), (I-a), or (I-b), or pharmaceutically acceptable salts thereof, include those in which:

(35)

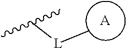

is selected from

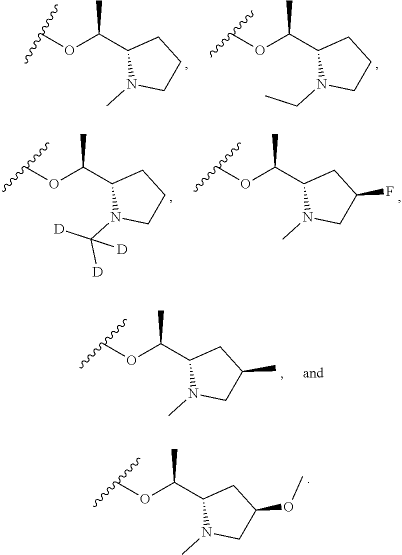

In addition to embodiments (1) through (35) in the preceding paragraphs, the compounds of Formula (I), (I-a), or (I-b), or pharmaceutically acceptable salts thereof, include those in which:

(36)

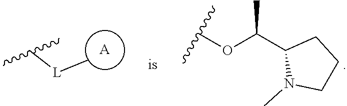

In some embodiments, the compound is selected from Table 1 or a pharmaceutically acceptable salt thereof.

TABLE 1

| Example | |
|---|---|
| No. | Structure |
| 1 | 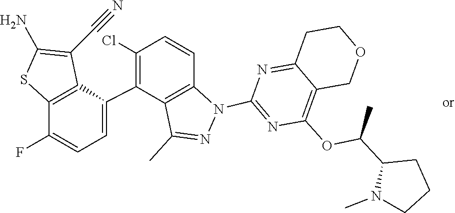 |
| | 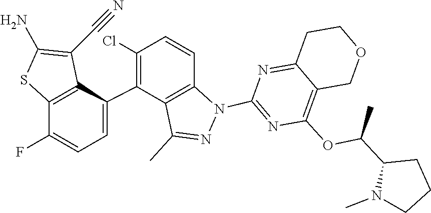 |
| 2 | 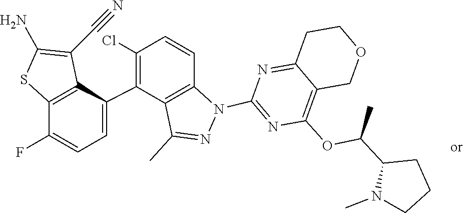 |
| | 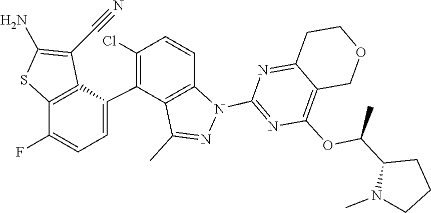 |
| 3 | 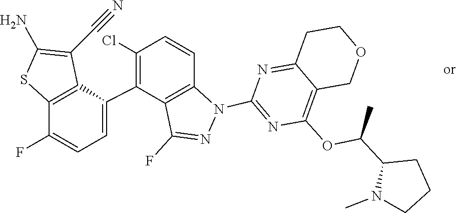 |
| | 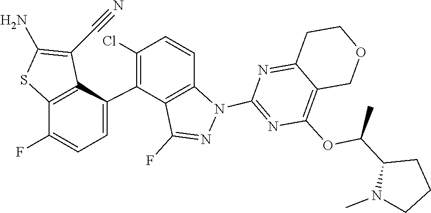 |
| 4 | 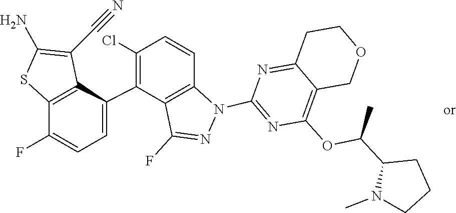 |
| | 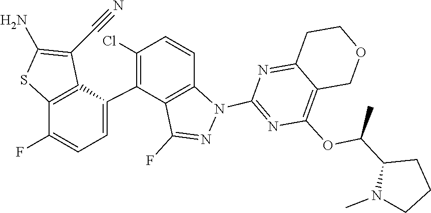 |
| 5 | 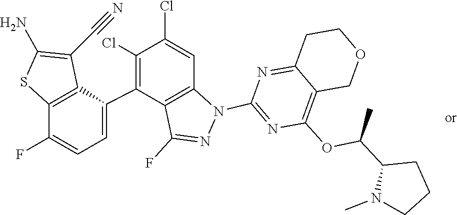 |
| | 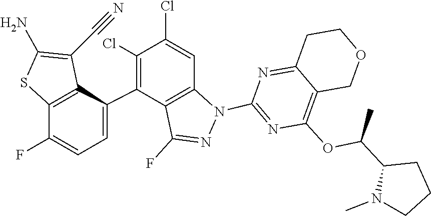 |
| 6 | 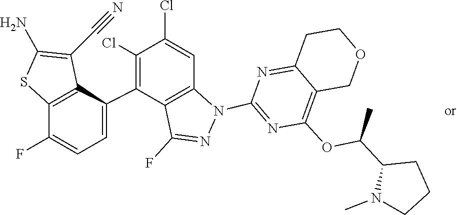 |
| | 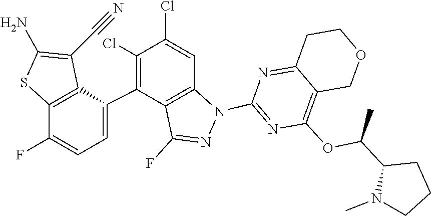 |
| 7 | 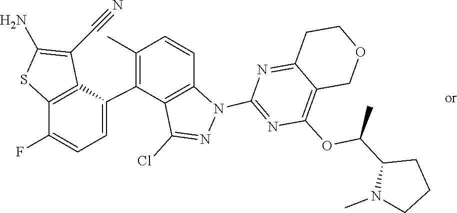 |
| | 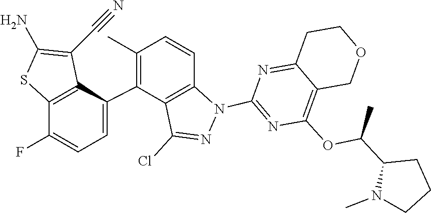 |
| 8 | 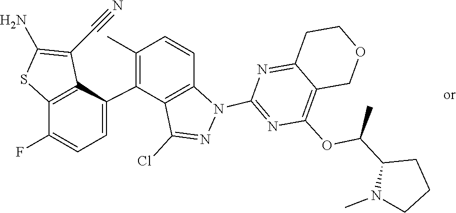 |
| | 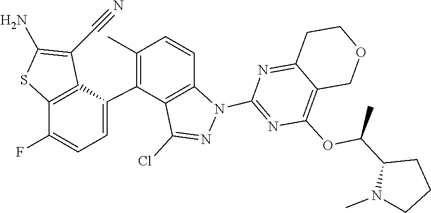 |
| 9 | 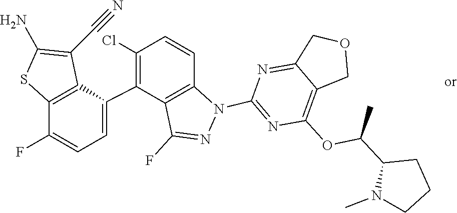 |
| | 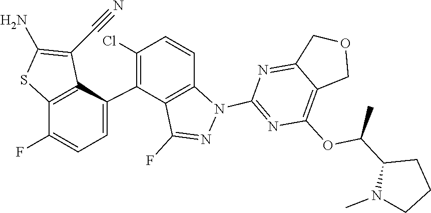 |
| 10 | 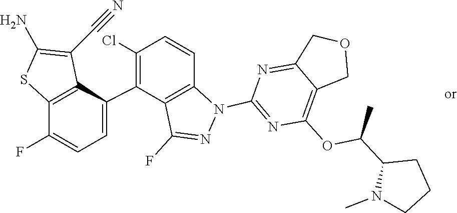 |
| | 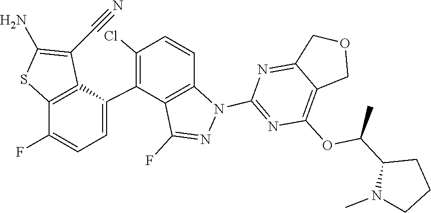 |
| 11 | 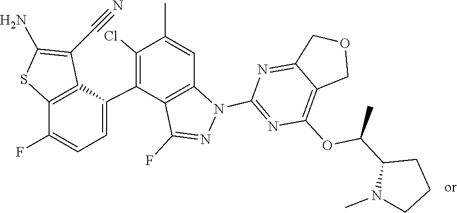 |
| | 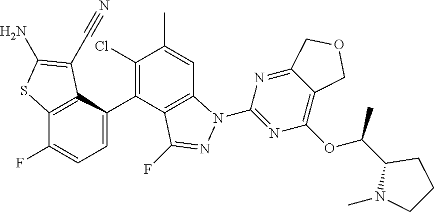 |
| 12 | 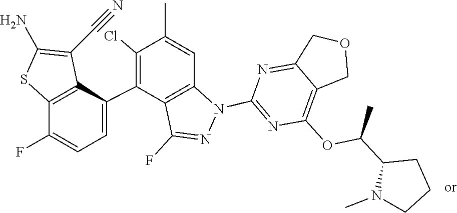 |
| | 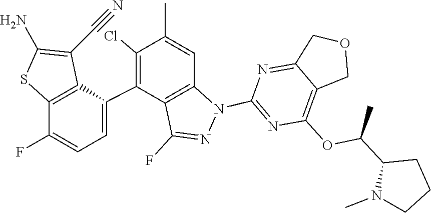 |
| 13 | 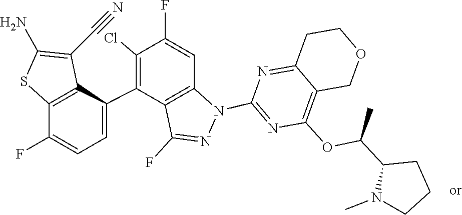 |
| | 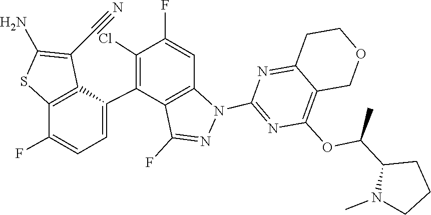 |
| 14 | 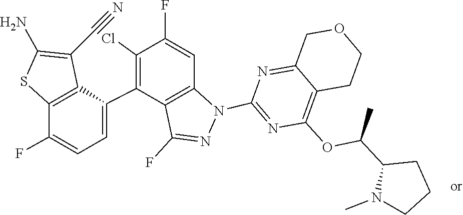 |
| | 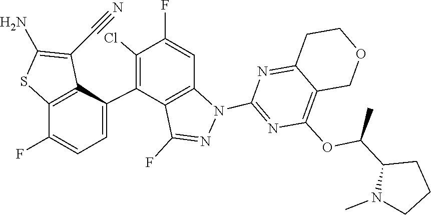 |
| 15 | 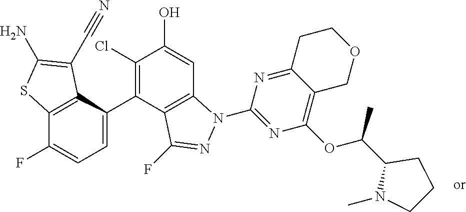 |
| | 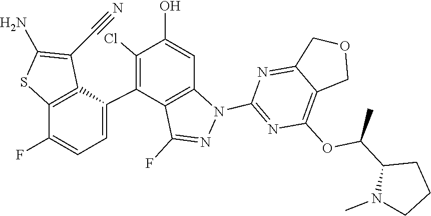 |
| 16 | 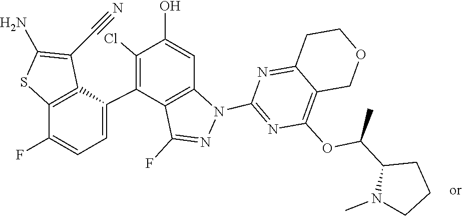 |
| | 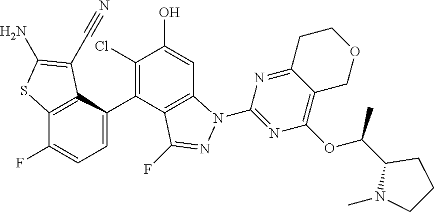 |
| 17 | 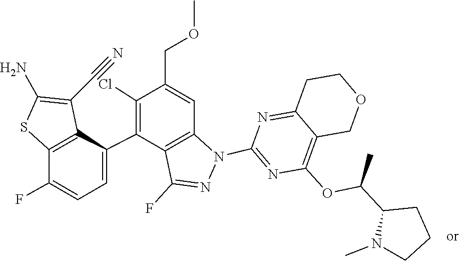 |
| | 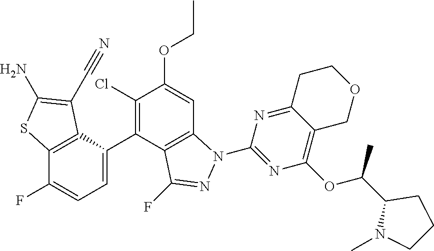 |
| 18 | 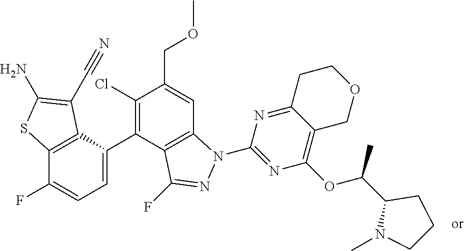 |
| | 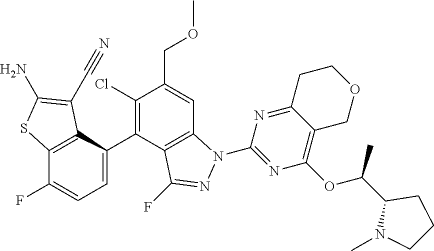 |
| 19 | 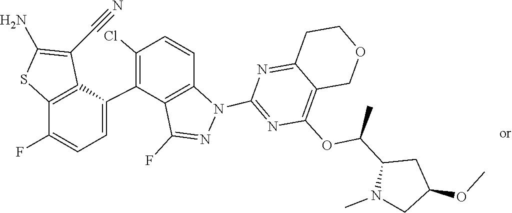 |
| | 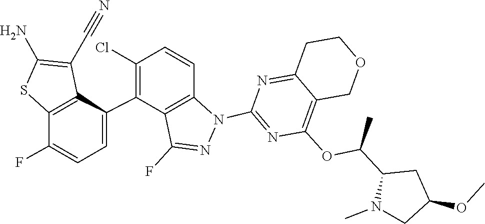 |
| 20 | 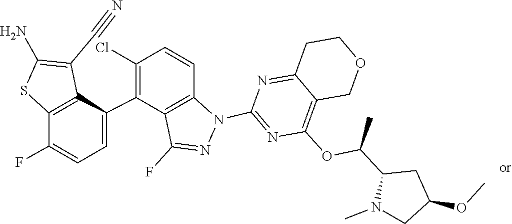 |
| | 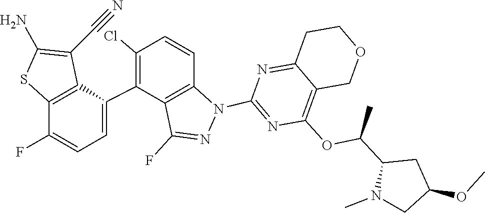 |
| 21 | 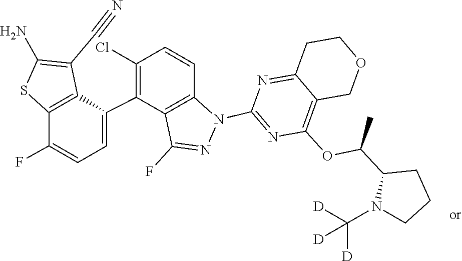 |
| | 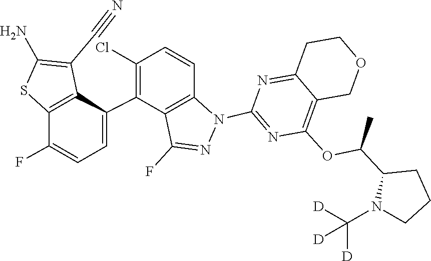 |
| 22 | 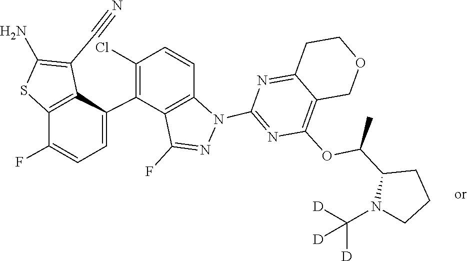 |
| | 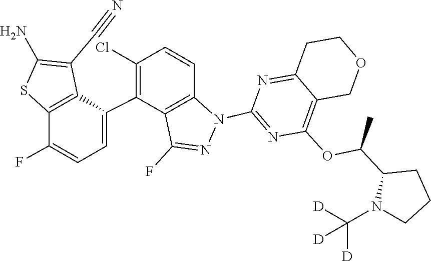 |
| 23 | 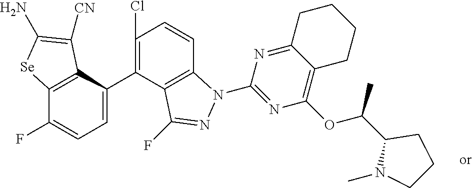 |
| | 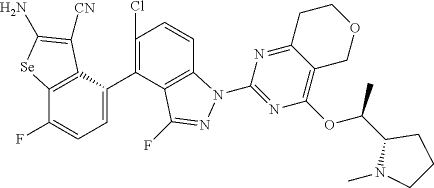 |
| 24 | 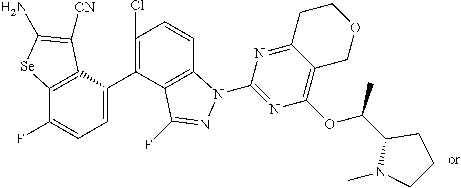 |
| | 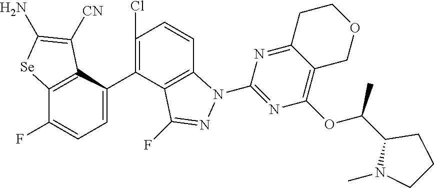 |
| 25 | 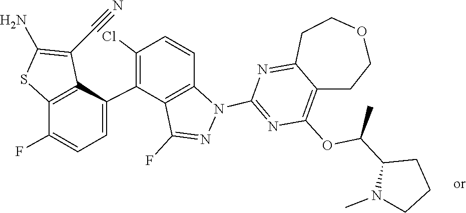 |
| | 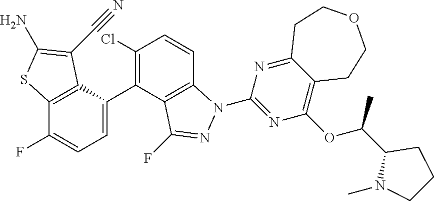 |
| 26 | 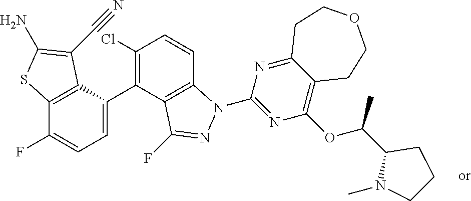 |
| | 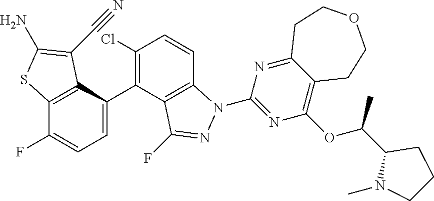 |
| 27 | 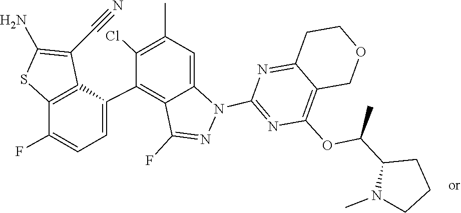 |
| | 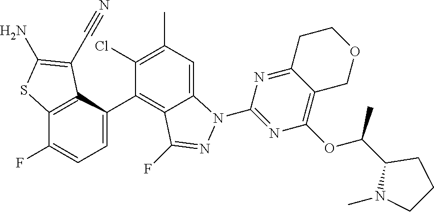 |
| 28 | 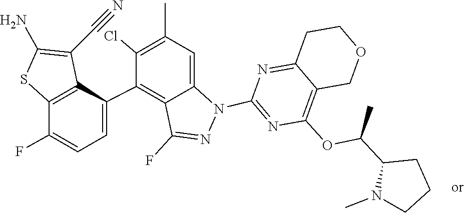 |
| | 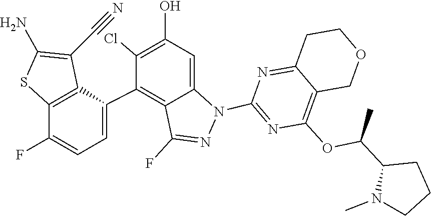 |
| 29 | 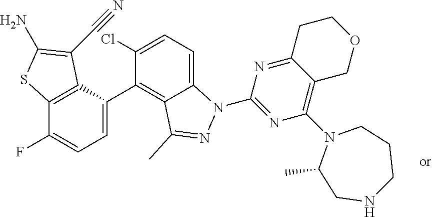 |
| | 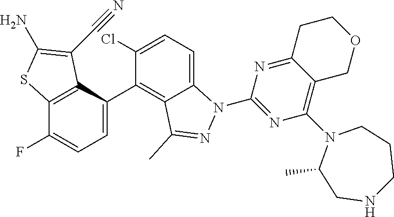 |
| 30 | 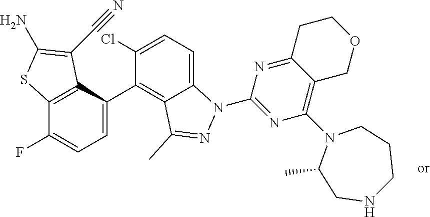 |
| | 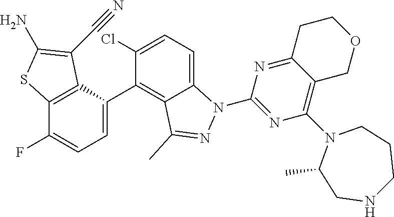 |
| 31 | 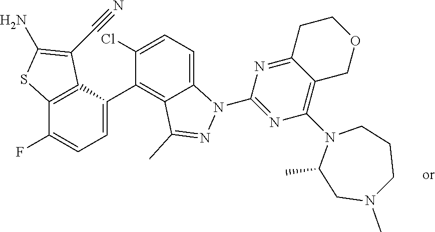 |
| | 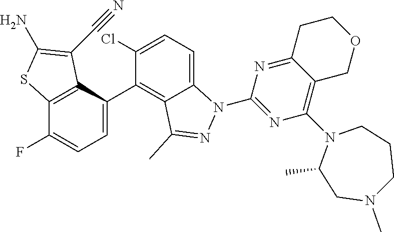 |
| 32 | 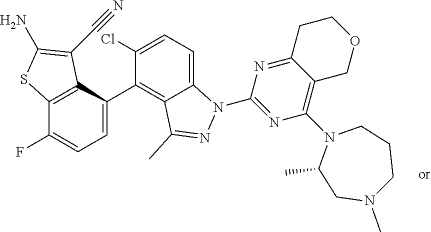 |
| | 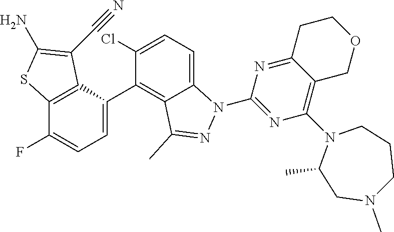 |
| 33 | 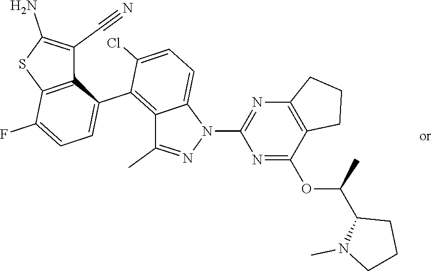 |
| | 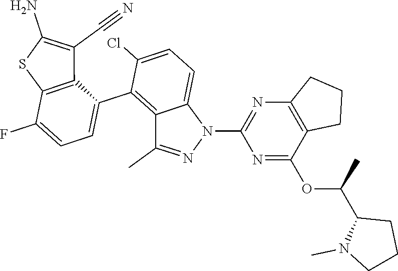 |
| 34 | 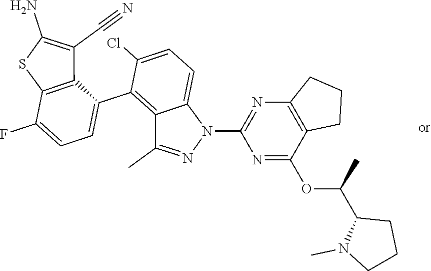 |
| | 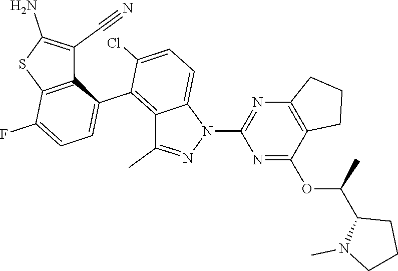 |
| 35 | 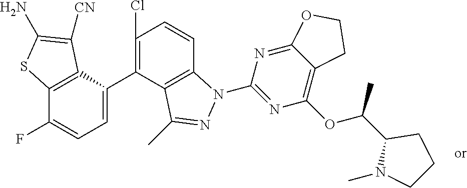 |
| | 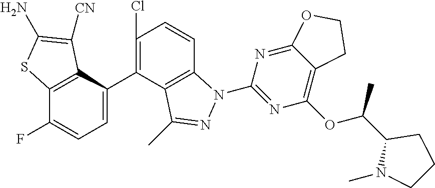 |
| 36 | 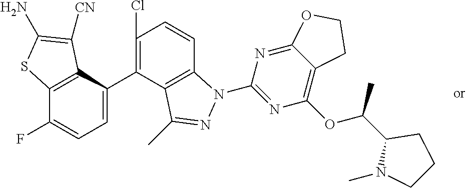 |
| | 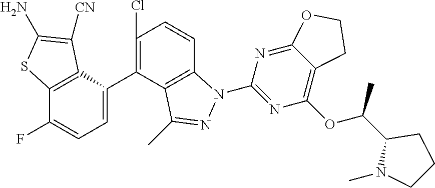 |
| 37 | 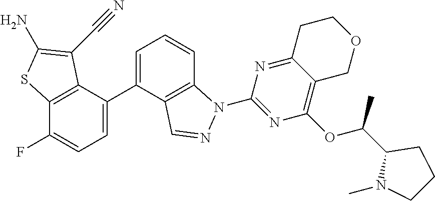 [SMILES: C[C@H](Oc1nc(-n2ncc3c(-c4ccc(F)c5sc(N)c(C#N)c45)cccc32)nc2c1COCC2)[C@@H]1CCCN1C] |
| 38 | 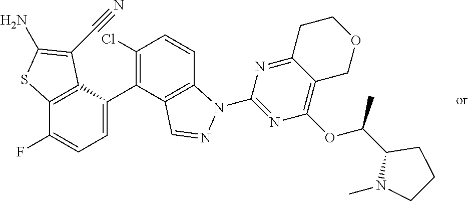 |
| | 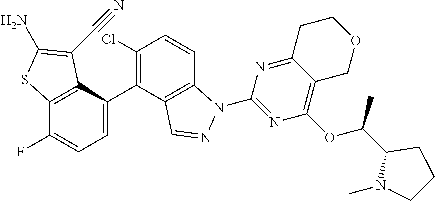 |
| 39 | 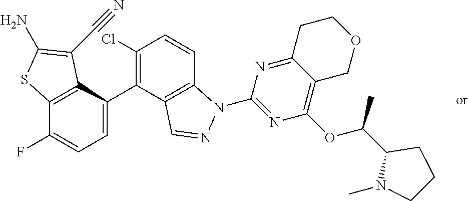 |
| | 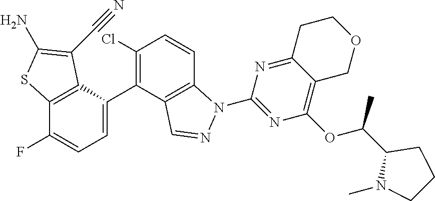 |
| 40 | 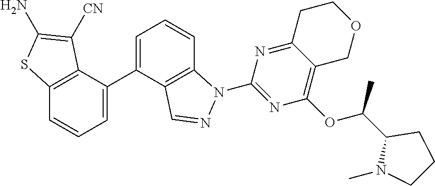 [SMILES: C[C@H](Oc1nc(-n2ncc3c(-c4cccc5sc(N)c(C#N)c45)cccc32)nc2c1COCC2)[C@@H]1CCCN1C] |
| 41 | 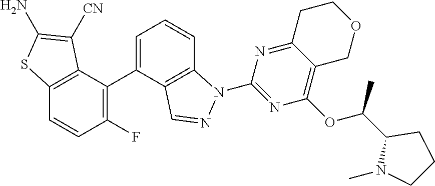 [SMILES: C[C@H](Oc1nc(-n2ncc3c(-c4c(F)ccc5sc(N)c(C#N)c45)cccc32)nc2c1COCC2)[C@@H]1CCCN1C] |
| 42 | 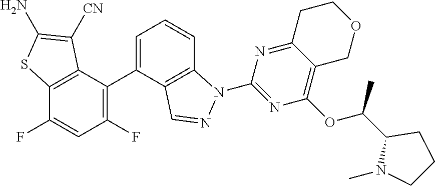 [SMILES: C[C@H](Oc1nc(-n2ncc3c(-c4c(F)cc(F)c5sc(N)c(C#N)c45)cccc32)nc2c1COCC2)[C@@H]1CCCN1C] |
| 43 | 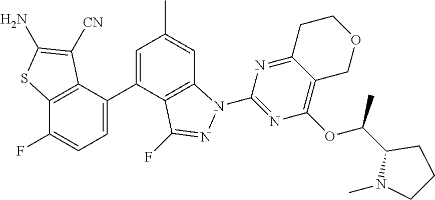 [SMILES: Cc1cc(-c2ccc(F)c3sc(N)c(C#N)c23)c2c(F)nn(-c3nc4c(c(O[C@@H](C)[C@@H]5CCCN5C)n3)COCC4)c2c1] |
| 44 | 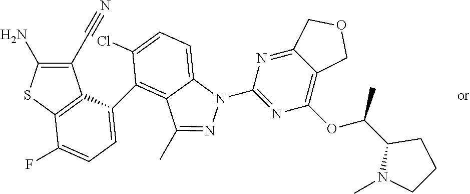 |
| | 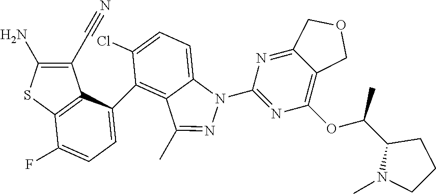 |
| 45 | 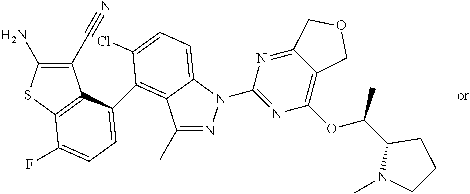 |
| | 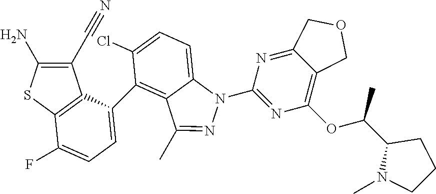 |
| 46 | 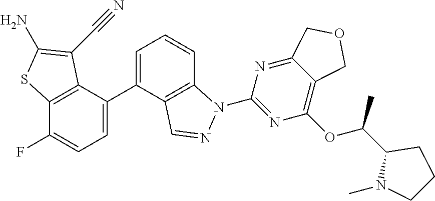 [SMILES: C[C@H](Oc1nc(-n2ncc3c(-c4ccc(F)c5sc(N)c(C#N)c45)cccc32)nc2c1COC2)[C@@H]1CCCN1C] |
| 47 | 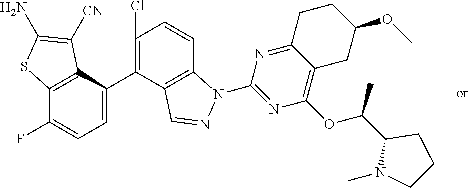 |
| | 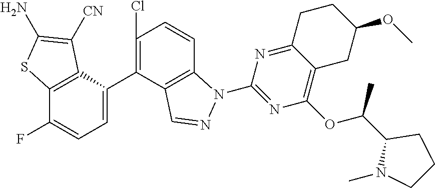 |
| | OR |
| | 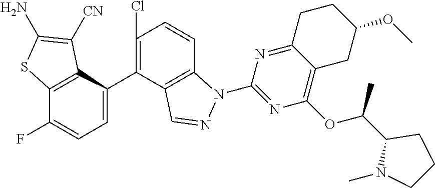 |
| | 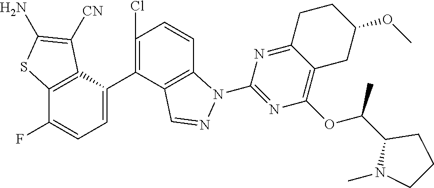 |
| 48 | 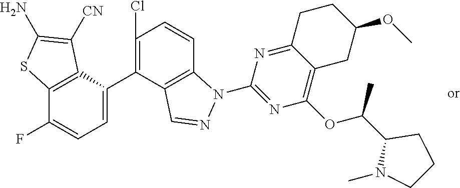 |
| | 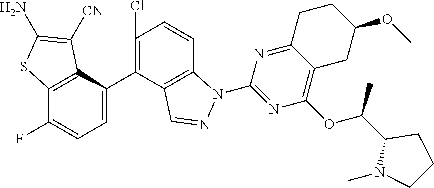 |
| | OR |
| | 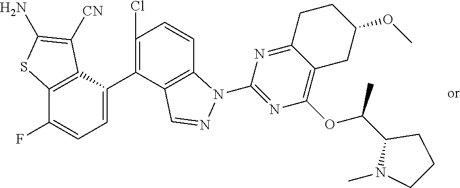 |
| | 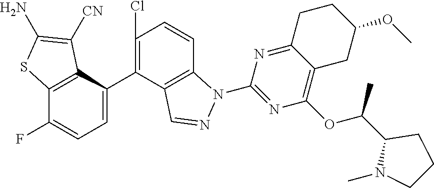 |
| 49 | 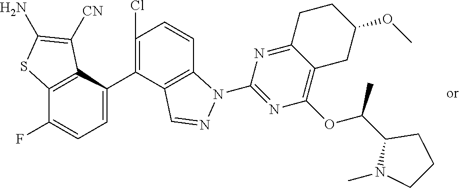 |
| | 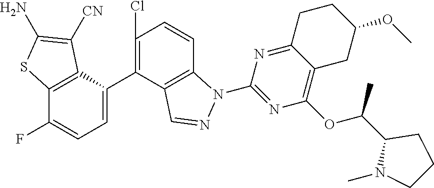 |
| | OR |
| | 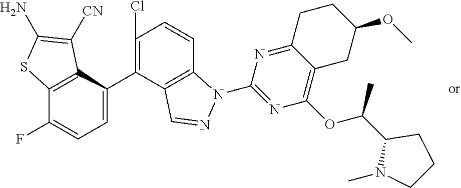 |
| | 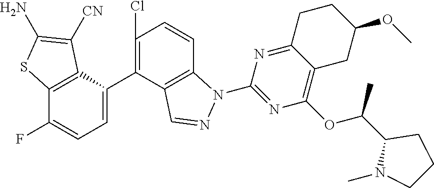 |
| 50 | 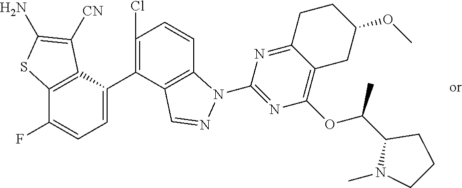 |
| | 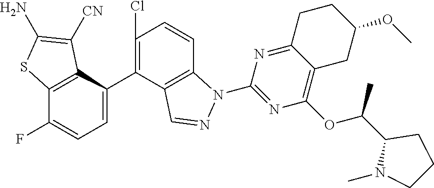 |
| | OR |
| | 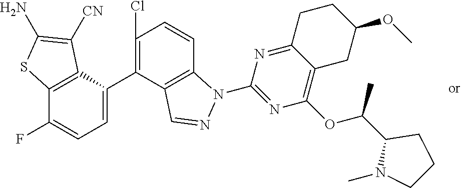 |
| | 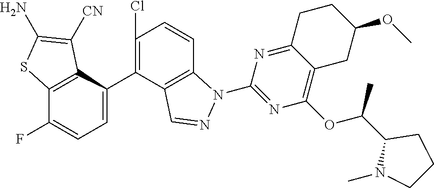 |
| 51 | 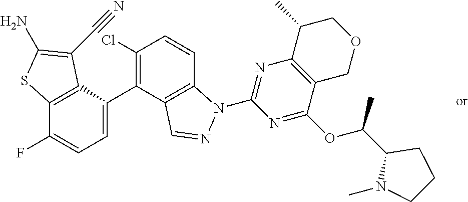 |
| | 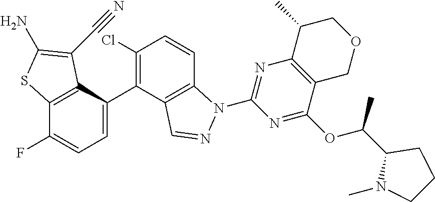 |
| | OR |
| | 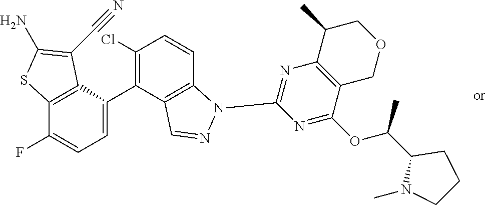 |
| | 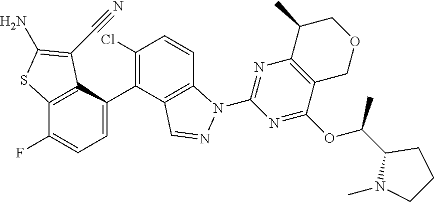 |
| 52 | 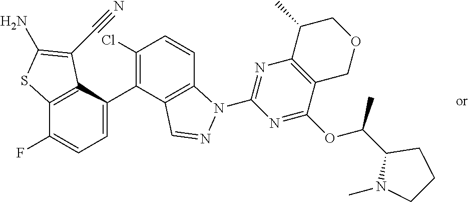 |
| | 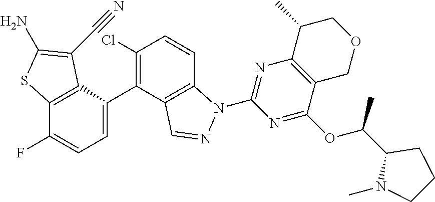 |
| | OR |
| | 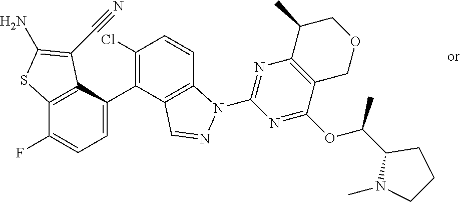 |
| | 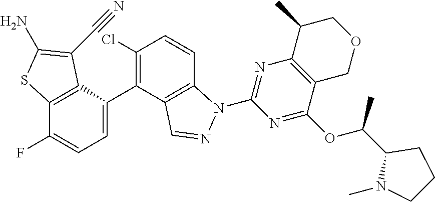 |
| 53 | 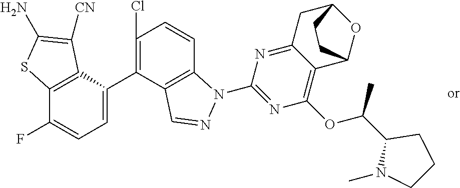 |
| | 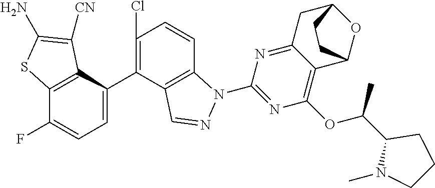 |
| | OR |
| | 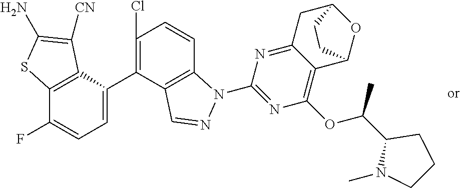 |
| | 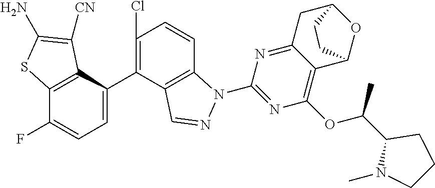 |
| 54 | 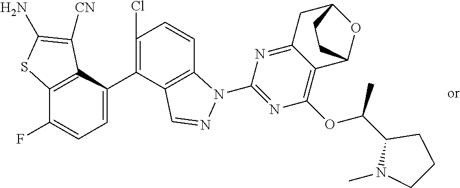 |
| | 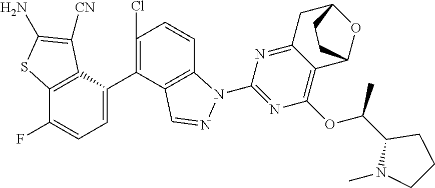 |
| | OR |
| | 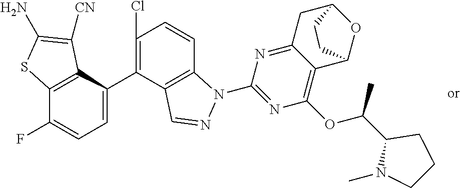 |
| | 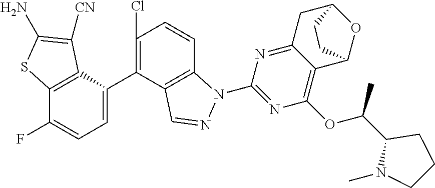 |
| 55 | 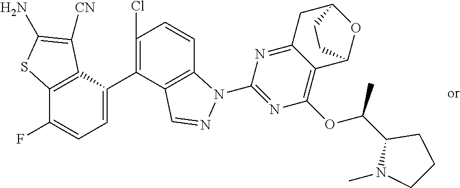 |
| | 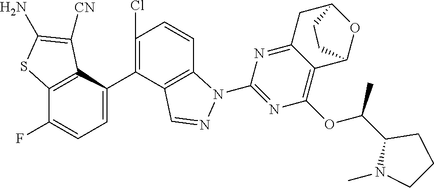 |
| | OR |
| | 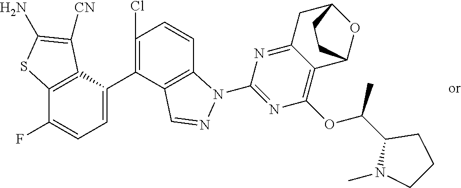 |
| | 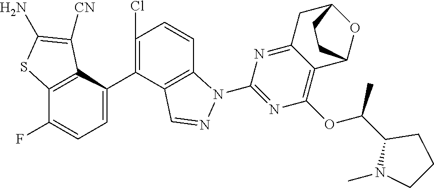 |
| 56 | 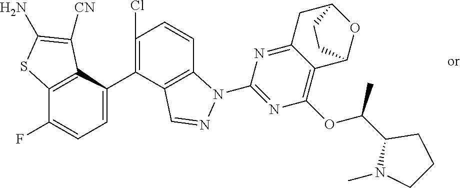 |
| | 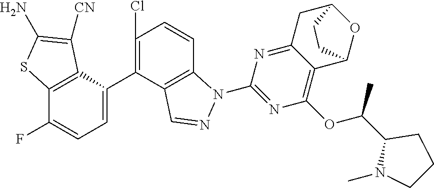 |
| | OR |
| | 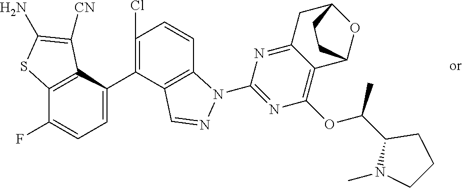 |
| | 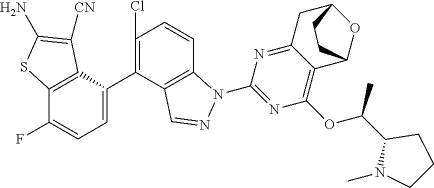 |
| 57 | 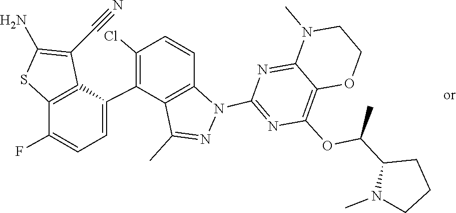 |
| | 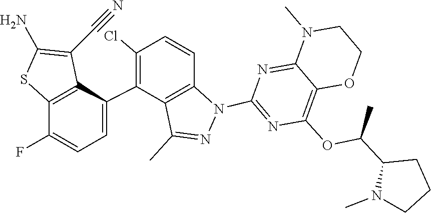 |
| 58 | 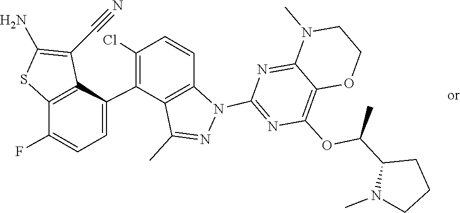 |
| | 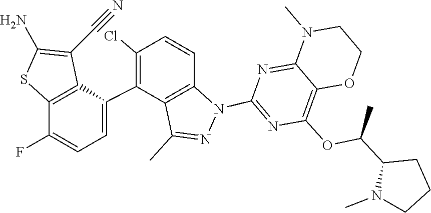 |
| 59 | 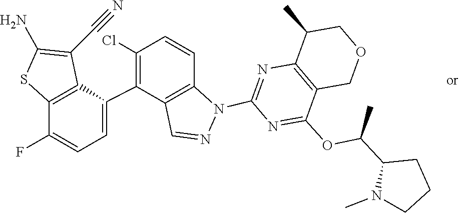 |
| | 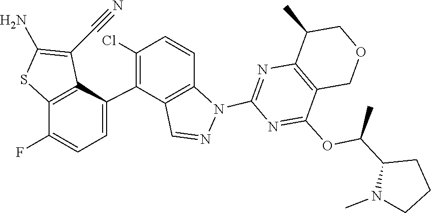 |
| | OR |
| | 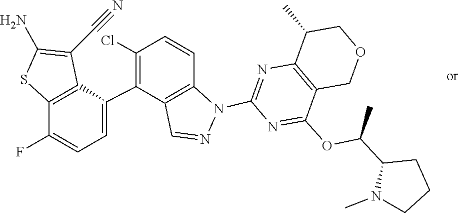 |
| | 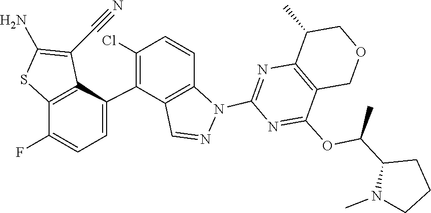 |
| 60 | 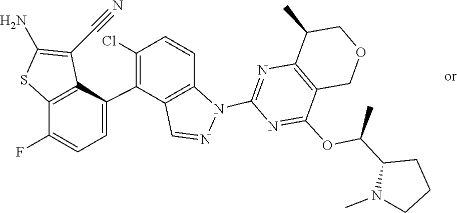 |
| | 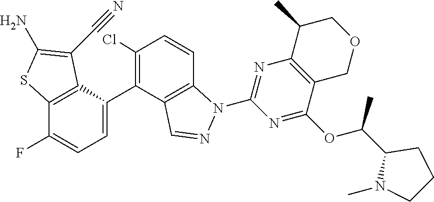 |
| | OR |
| | 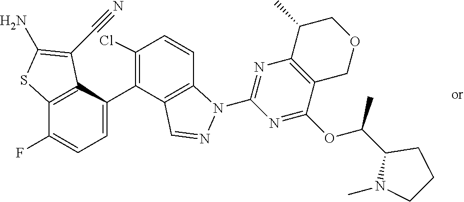 |
| | 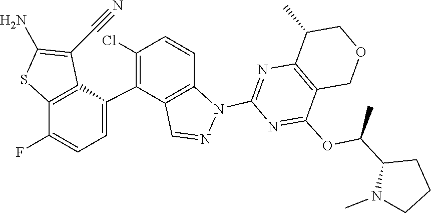 |
| 61 | 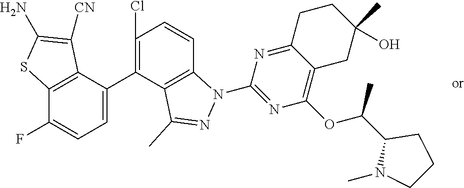 [SMILES: Cc1nn(-c2nc3c(c(O[C@@H](C)[C@@H]4CCCN4C)n2)C[C@@](C)(O)CC3)c2ccc(Cl)c(-c3ccc(F)c4sc(N)c(C#N)c34)c12] |
| | 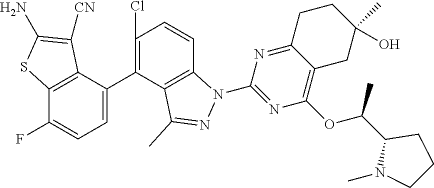 [SMILES: Cc1nn(-c2nc3c(c(O[C@@H](C)[C@@H]4CCCN4C)n2)C[C@](C)(O)CC3)c2ccc(Cl)c(-c3ccc(F)c4sc(N)c(C#N)c34)c12] |
| 62 | 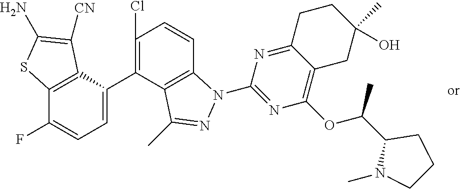 |
| | 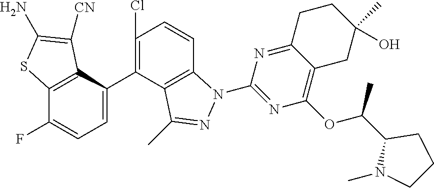 |
| | OR |
| | 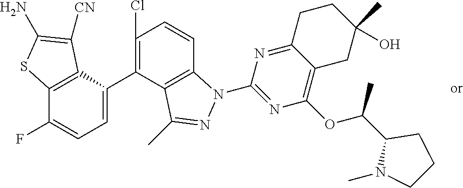 |
| | 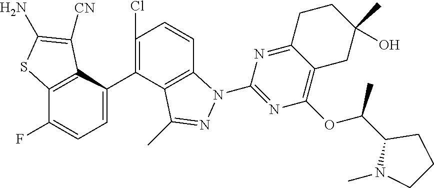 |
| 63 | 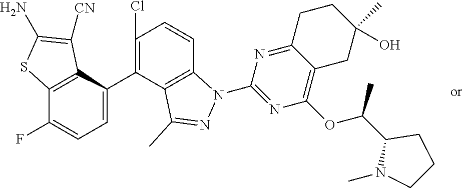 |
| | 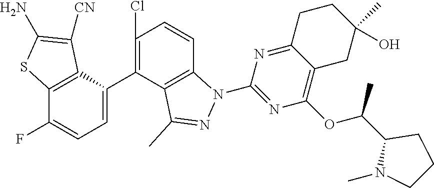 |
| | OR |
| | 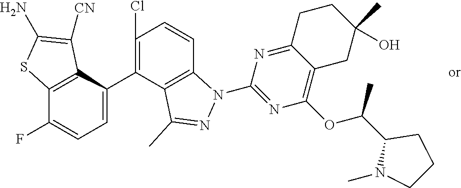 |
| | 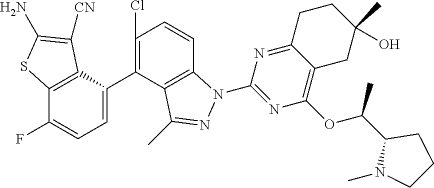 |
| 64 | 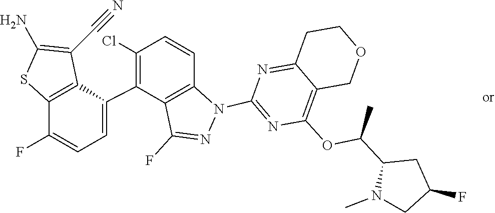 |
| | 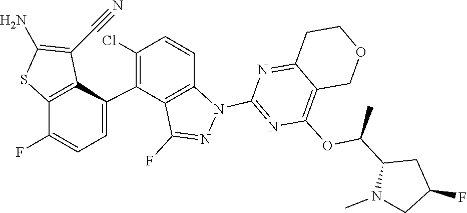 |
| 65 | 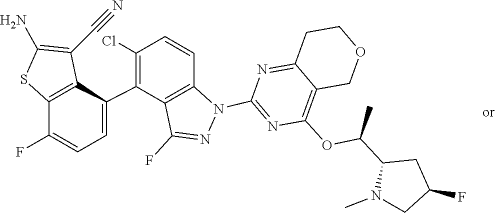 |
| | 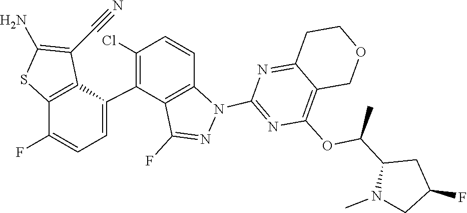 |
| 66 | 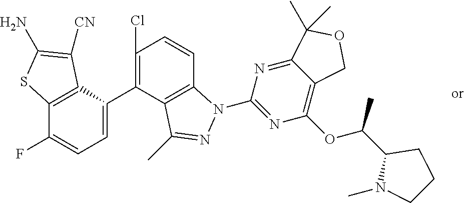 |
| | 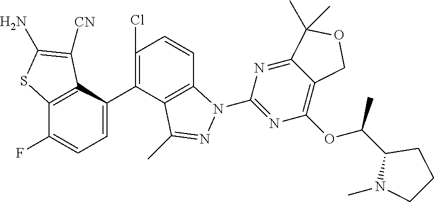 |
| 67 | 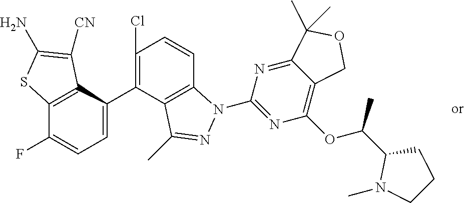 |
| | 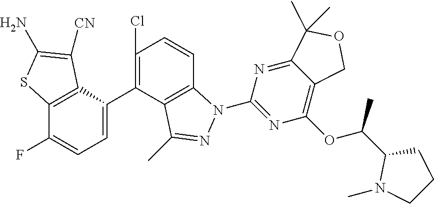 |
| 68 | 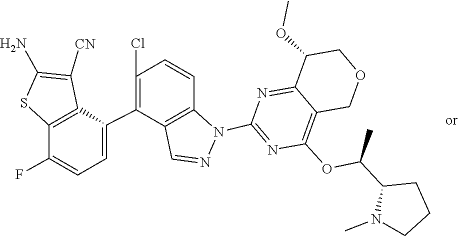 |
| | 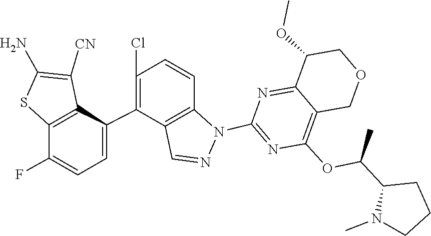 |
| | OR |
| | 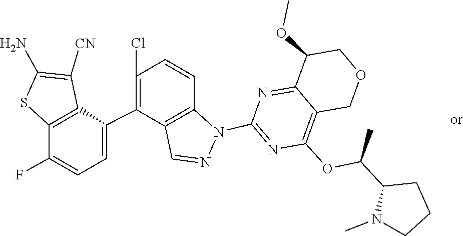 |
| | 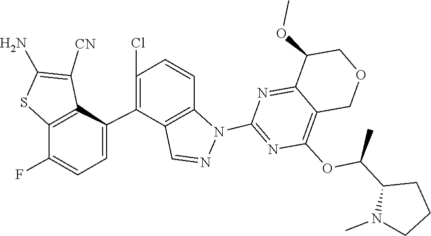 |
| 69 | 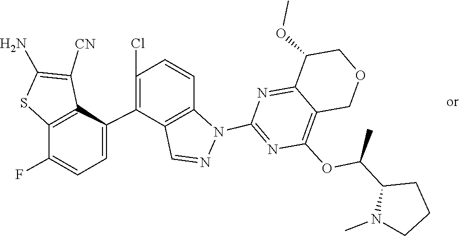 |
| | 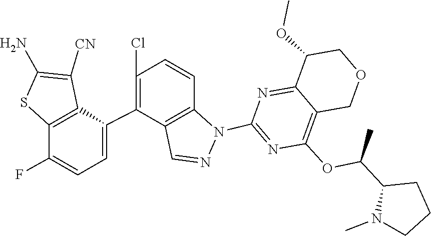 |
| | OR |
| | 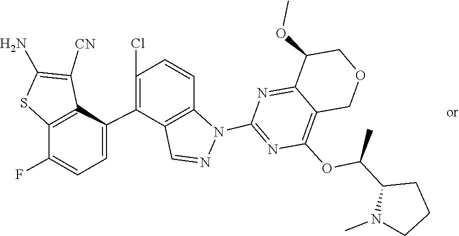 |
| | 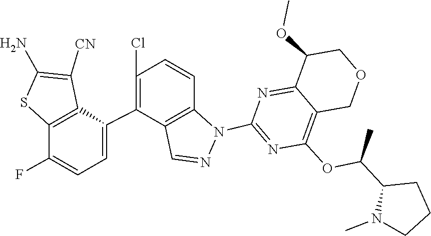 |
| 70 | 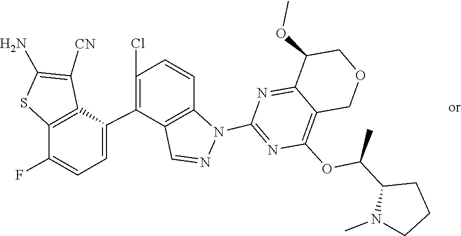 |
| | 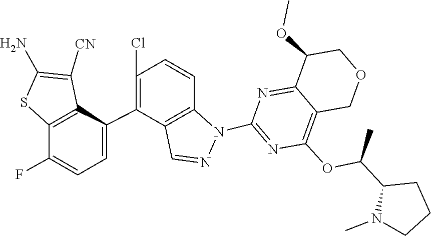 |
| | OR |
| | 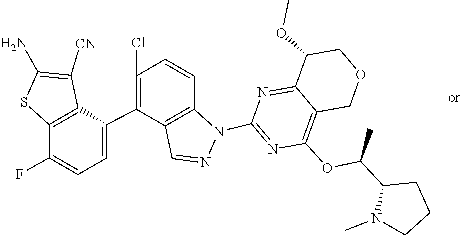 |
| | 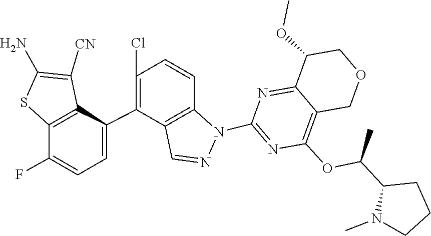 |
| 71 | 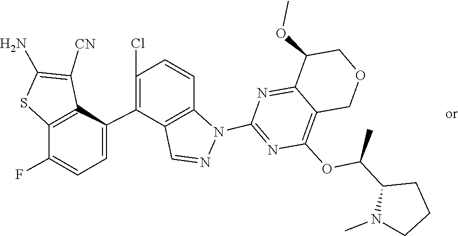 |
| | 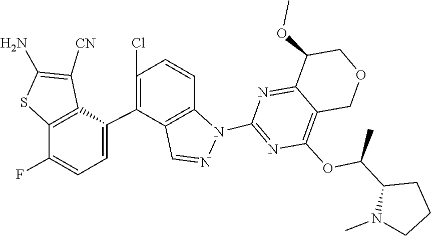 |
| | OR |
| | 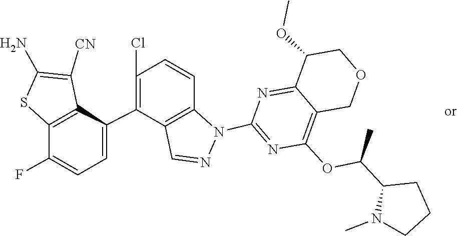 |
| | 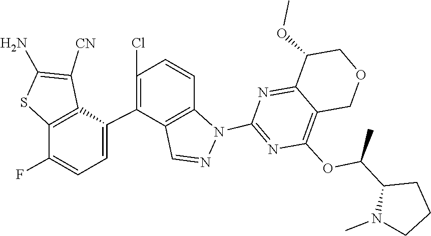 |
| 72 | 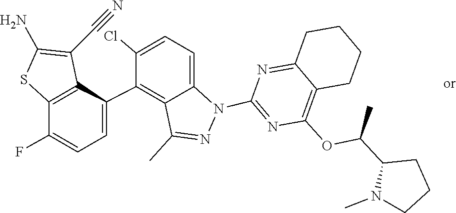 |
| | 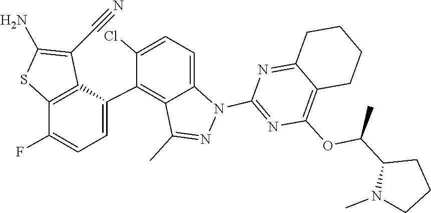 |
| 73 | 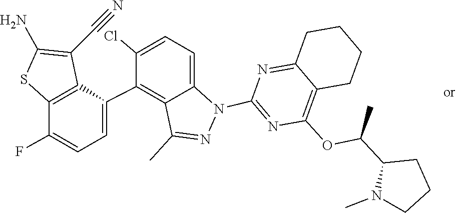 |
| | 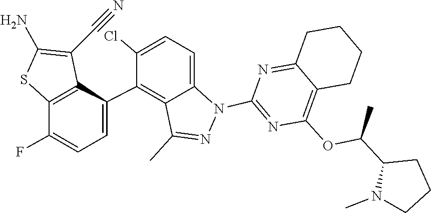 |
| 74 | 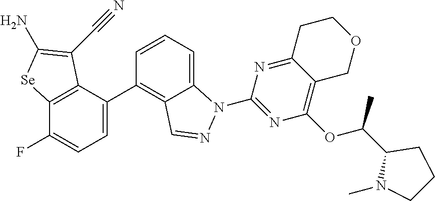 [SMILES: C[C@H](Oc1nc(-n2ncc3c(-c4ccc(F)c5[se]c(N)c(C#N)c45)cccc32)nc2c1COCC2)[C@@H]1CCCN1C] |
| 75 | 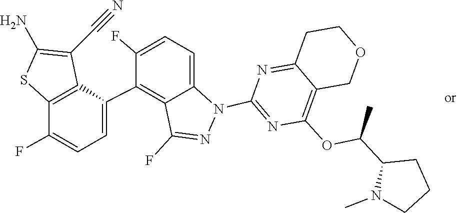 |
| | 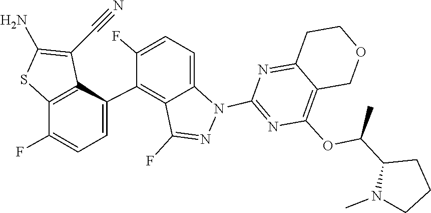 |
| 76 | 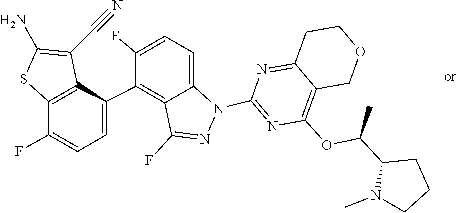 |
| | 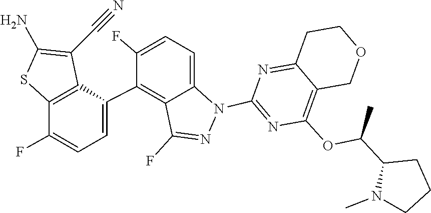 |
| 77 | 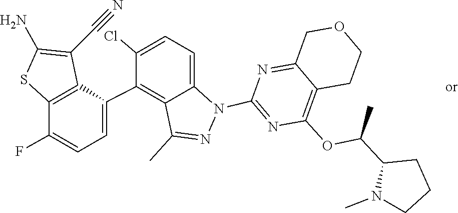 |
| | 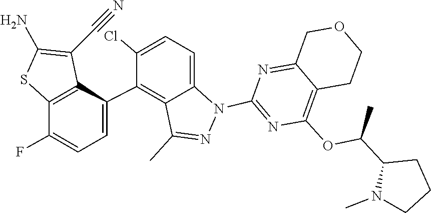 |
| 78 | 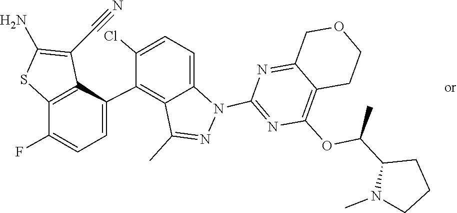 |
| | 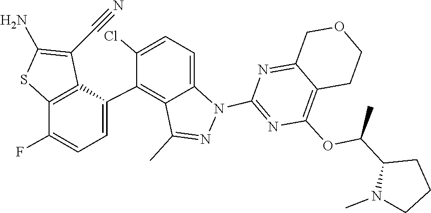 |
| 79 | 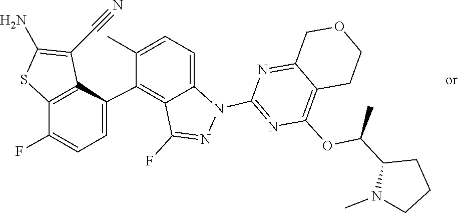 |
| | 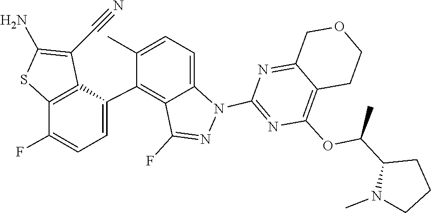 |
| 80 | 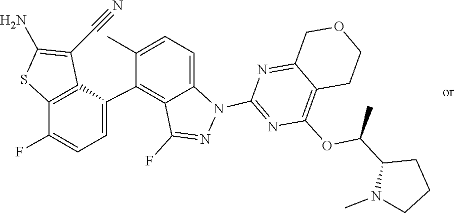 |
| | 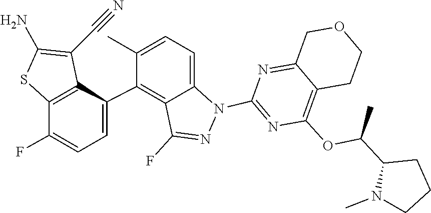 |
| 81 | 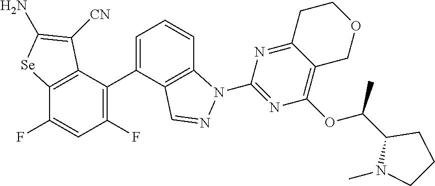 [SMILES: C[C@H](Oc1nc(-n2ncc3c(-c4c(F)cc(F)c5[se]c(N)c(C#N)c45)cccc32)nc2c1COCC2)[C@@H]1CCCN1C] |
| 82 | 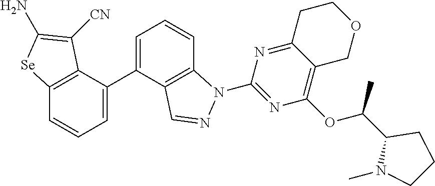 [SMILES: C[C@H](Oc1nc(-n2ncc3c(-c4cccc5[se]c(N)c(C#N)c45)cccc32)nc2c1COCC2)[C@@H]1CCCN1C] |
| 83 | 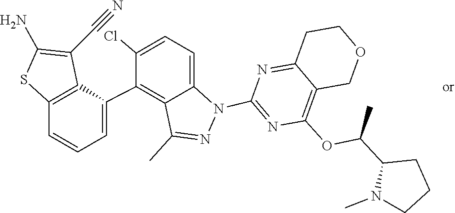 |
| | 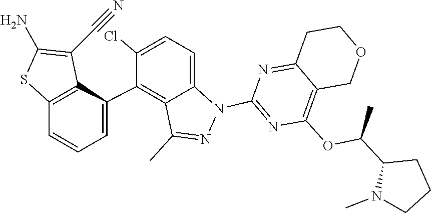 |
| 84 | 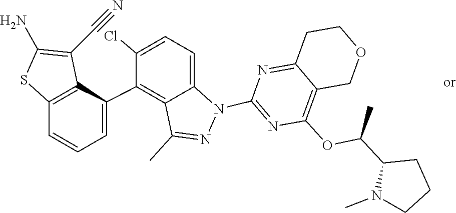 |
| | 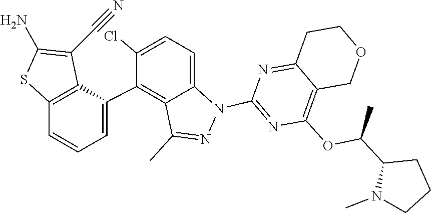 |
| 85 | 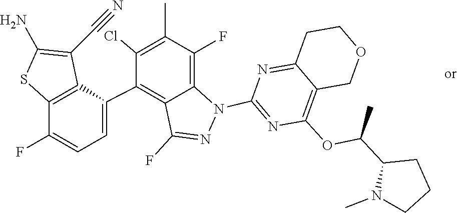 |
| | 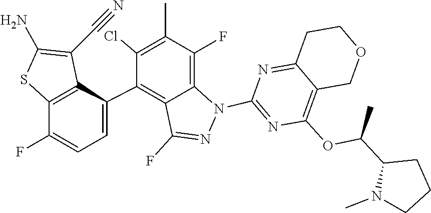 |
| 86 | 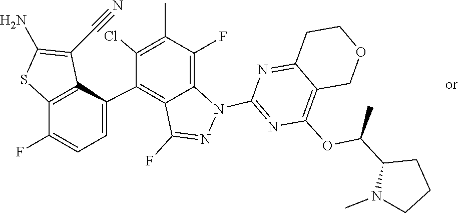 |
| | 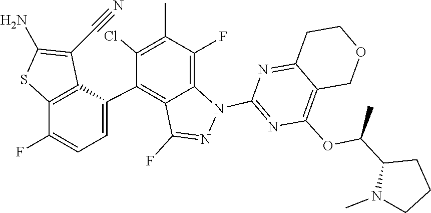 |
| 87 | 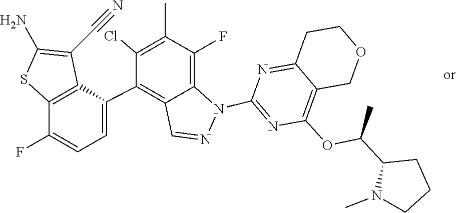 |
| | 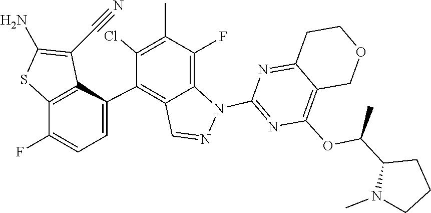 |
| 88 | 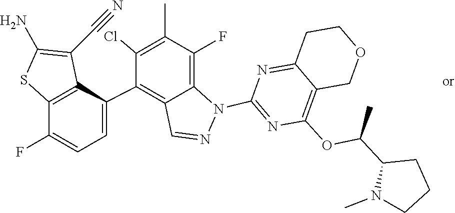 |
| | 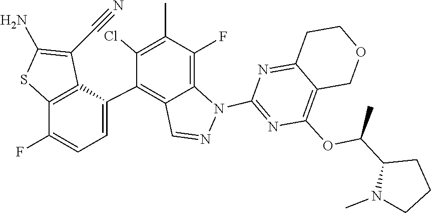 |
| 89 | 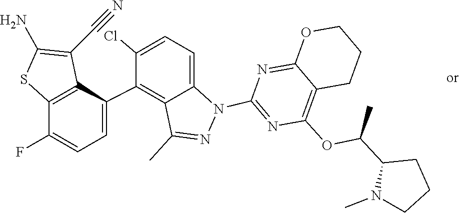 |
| | 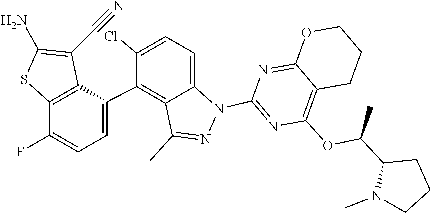 |
| 90 | 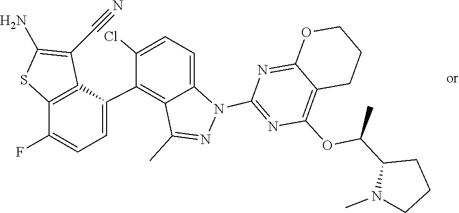 |
| | 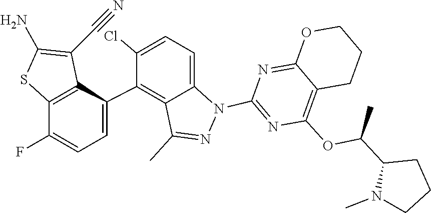 |
| 91 | 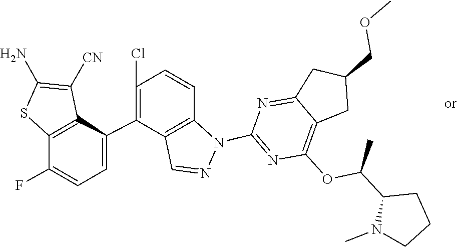 |
| | 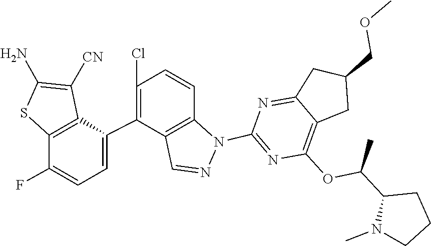 |
| | OR |
| | 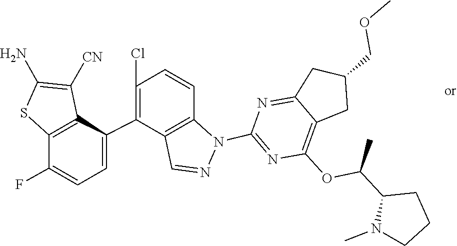 |
| | 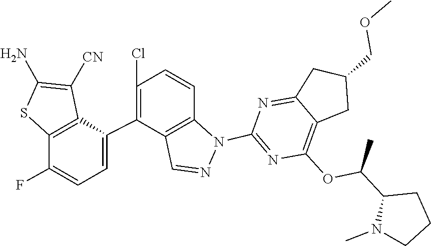 |
| 92 | 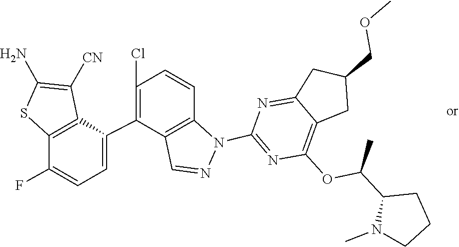 |
| | 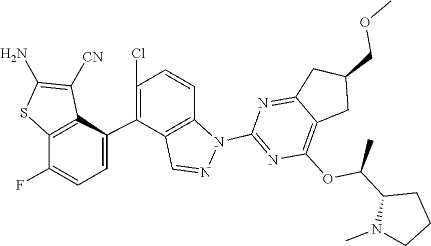 |
| | OR |
| | 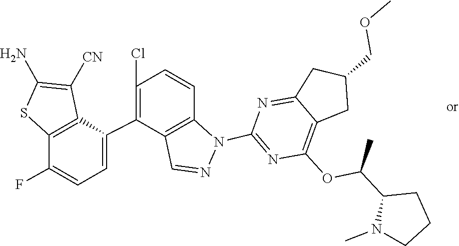 |
| | 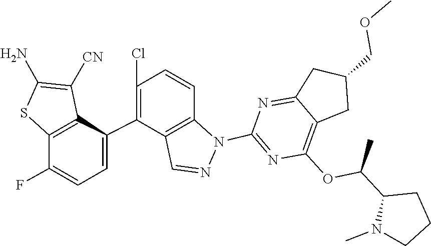 |
| 93 | 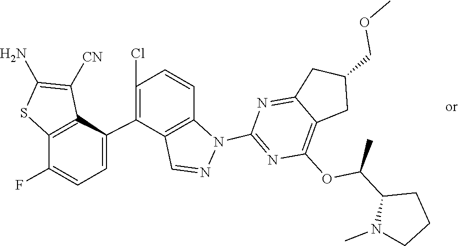 |
| | 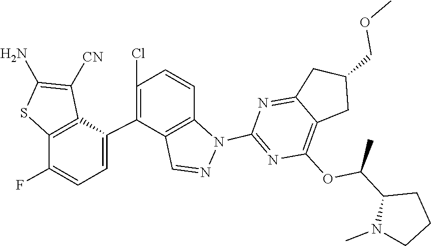 |
| | OR |
| | 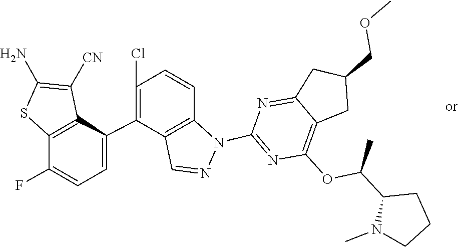 |
| | 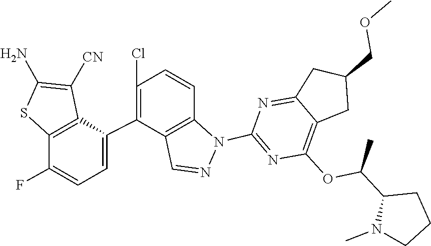 |
| 94 | 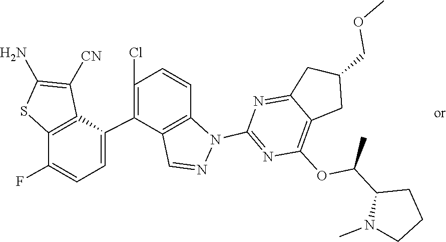 |
| | 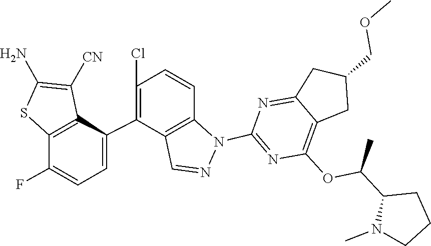 |
| | OR |
| | 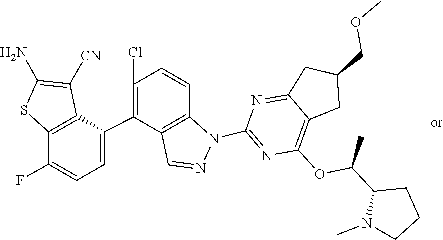 |
| | 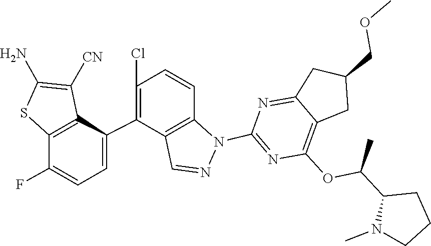 |
| 95 | 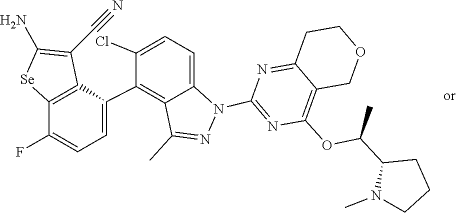 |
| | 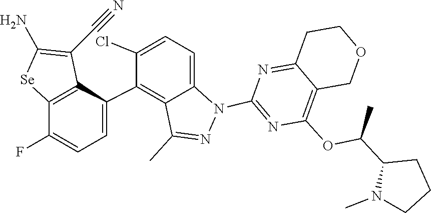 |
| 96 | 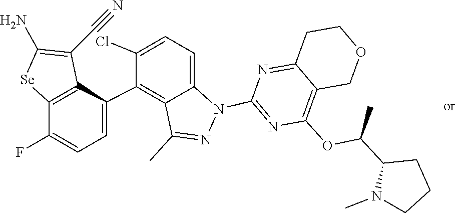 |
| | 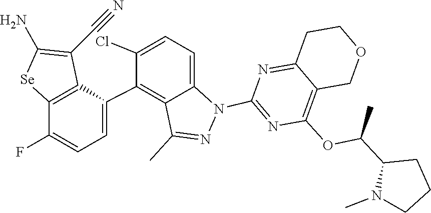 |
| 97 | 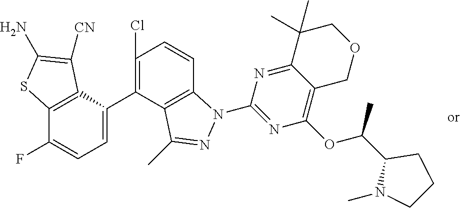 |
| | 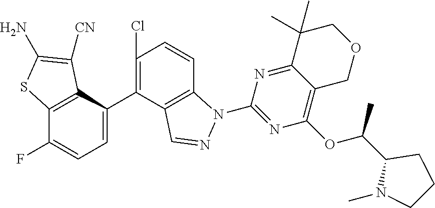 |
| 98 | 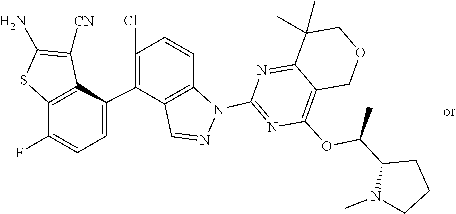 |
| | 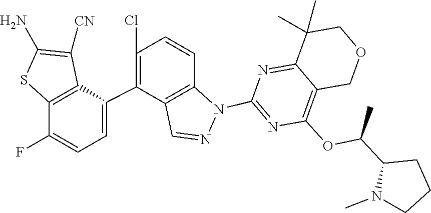 |
| 99 | 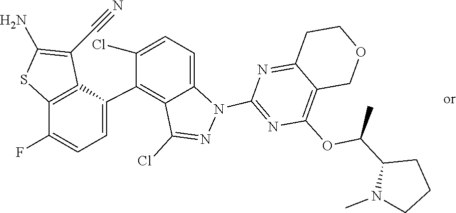 |
| | 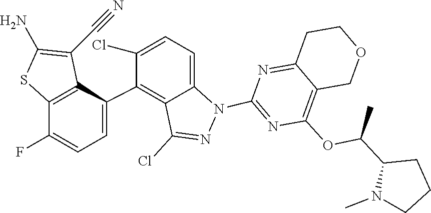 |
| 100 | 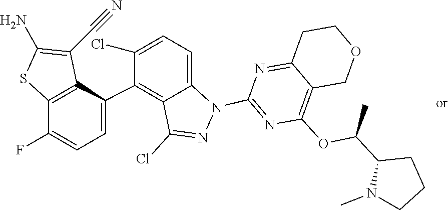 |
| | 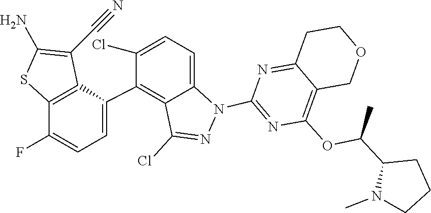 |
| 101 | 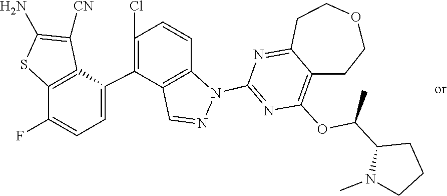 |
| | 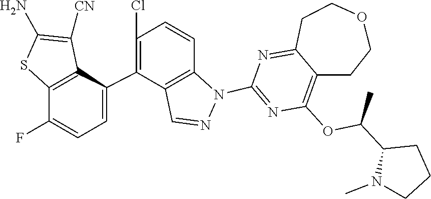 |
| 102 | 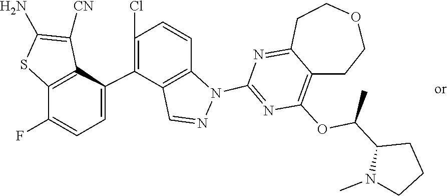 |
| | 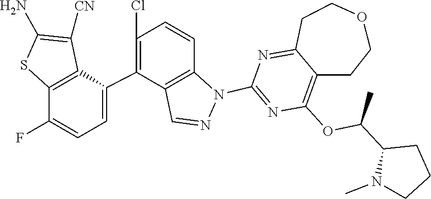 |
| 103 | 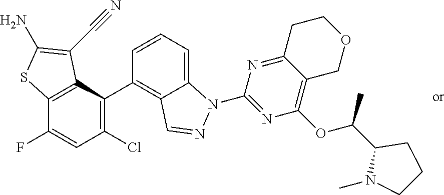 |
| | 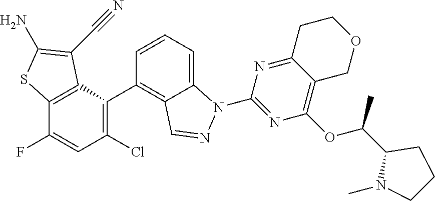 |
| 104 | 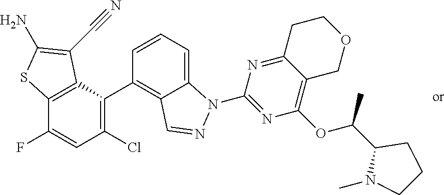 |
| | 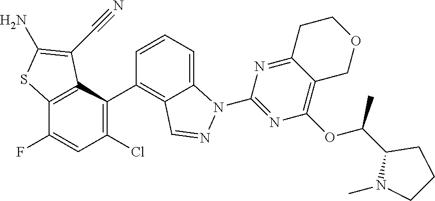 |
| 105 | 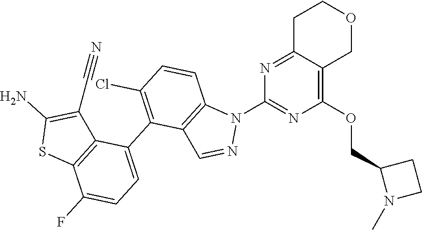 [SMILES: CN1CC[C@@H]1COc1nc(-n2ncc3c(-c4ccc(F)c5sc(N)c(C#N)c45)c(Cl)ccc32)nc2c1COCC2] |
| 106 | 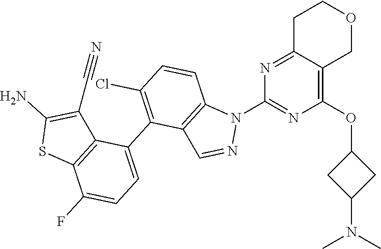 [SMILES: CN(C)C1CC(Oc2nc(-n3ncc4c(-c5ccc(F)c6sc(N)c(C#N)c56)c(Cl)ccc43)nc3c2COCC3)C1] |
| 107 | 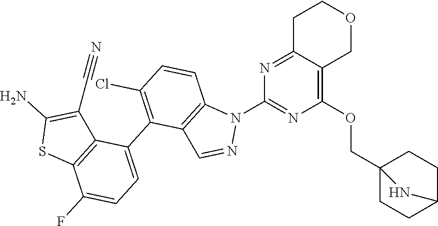 [SMILES: N#Cc1c(N)sc2c(F)ccc(-c3c(Cl)ccc4c3cnn4-c3nc4c(c(OCC56CCC(CC5)N6)n3)COCC4)c12] |
| 108 | 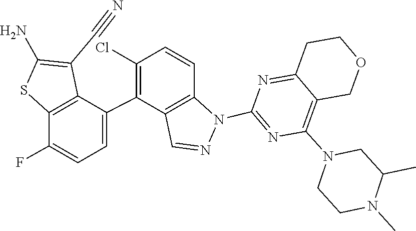 [SMILES: CC1CN(c2nc(-n3ncc4c(-c5ccc(F)c6sc(N)c(C#N)c56)c(Cl)ccc43)nc3c2COCC3)CCN1C] |
| 109 | 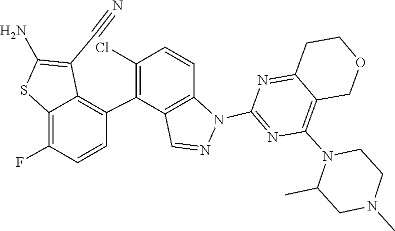 [SMILES: CC1CN(C)CCN1c1nc(-n2ncc3c(-c4ccc(F)c5sc(N)c(C#N)c45)c(Cl)ccc32)nc2c1COCC2] |
| 110 | 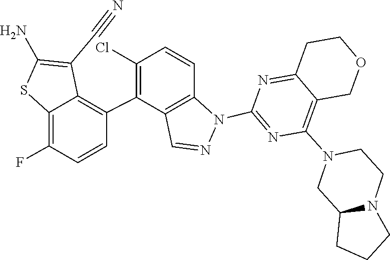 [SMILES: N#Cc1c(N)sc2c(F)ccc(-c3c(Cl)ccc4c3cnn4-c3nc4c(c(N5CCN6CCC[C@H]6C5)n3)COCC4)c12] |
| 111 | 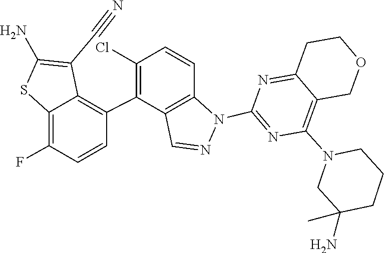 [SMILES: CC1(N)CCCN(c2nc(-n3ncc4c(-c5ccc(F)c6sc(N)c(C#N)c56)c(Cl)ccc43)nc3c2COCC3)C1] |
| 112 | 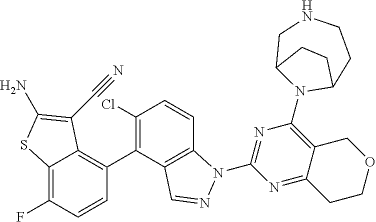 [SMILES: N#Cc1c(N)sc2c(F)ccc(-c3c(Cl)ccc4c3cnn4-c3nc4c(c(N5C6CCNCC5CC6)n3)COCC4)c12] |
| 113 | 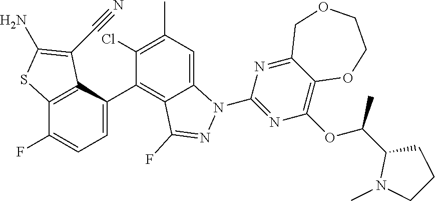 |
| 114 | 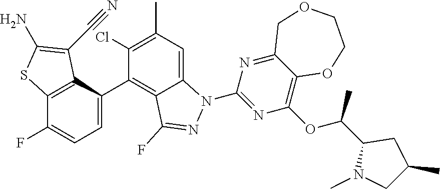 |
| 115 | 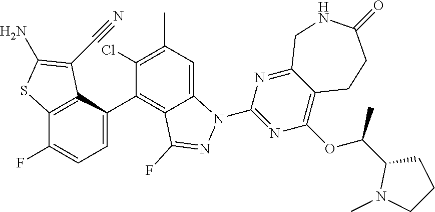 |
| 28a | 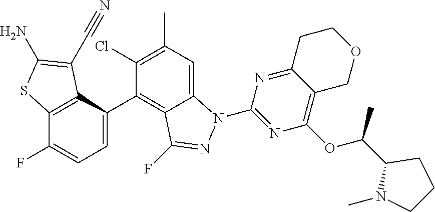 |
| 12a | 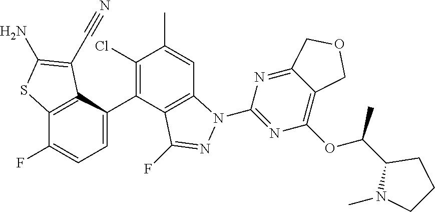 |
| 117 | 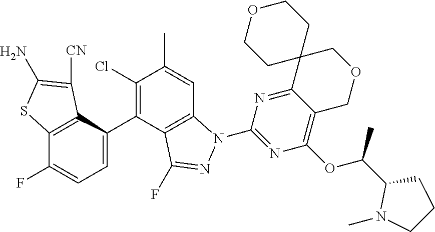 |
| 118 | 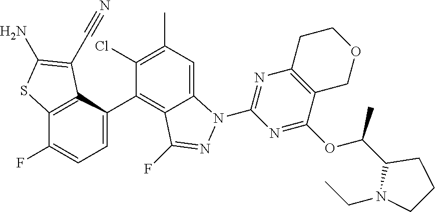 |
| 119 | 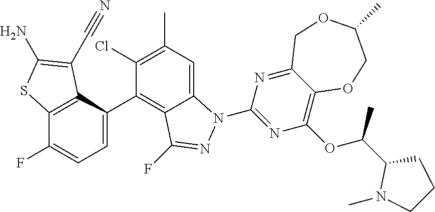 |
| 120 | 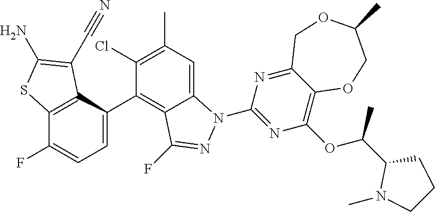 |
| 121 | 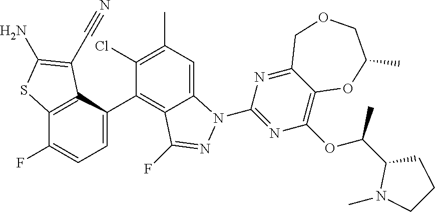 |
| 122 | 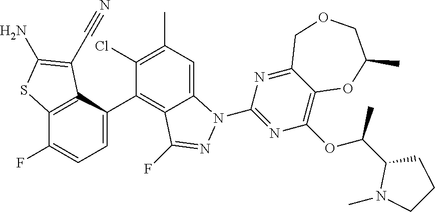 |
| 123 | 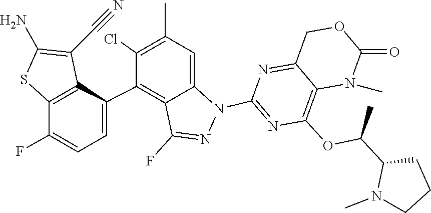 |
| 124 | 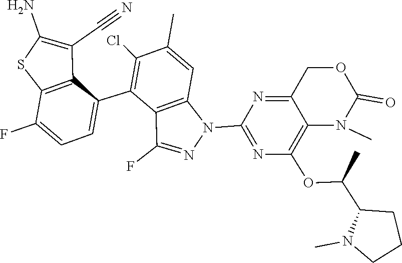 |
| 125 | 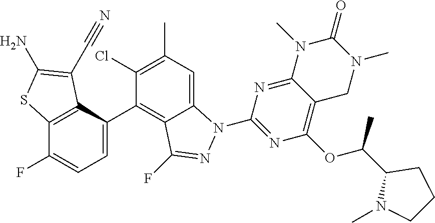 |
| 126 | 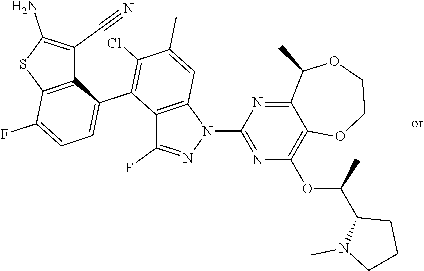 |
| | 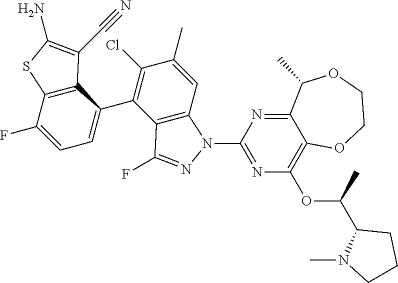 |
| 127 | 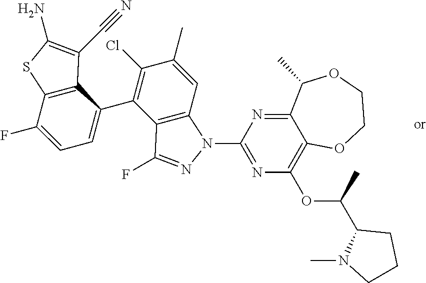 |
| | 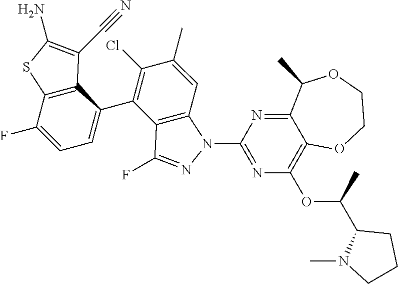 |
| 128 | 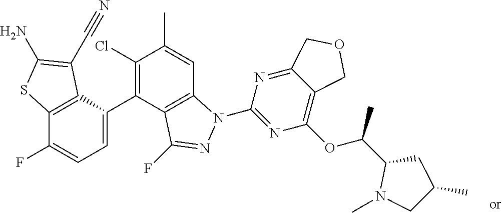 |
| | 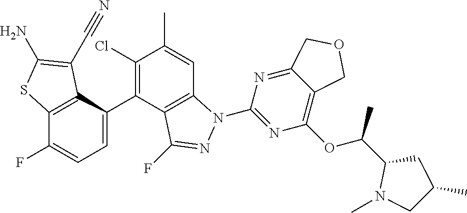 |
| 129 | 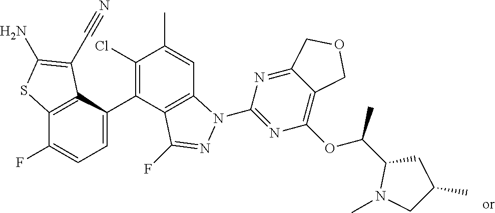 |
| | 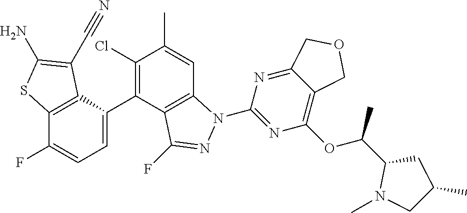 |
| 130 | 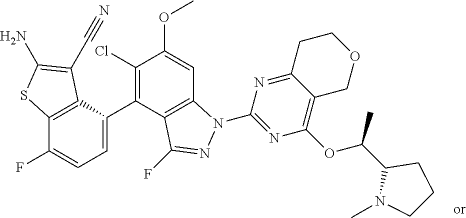 |
| | 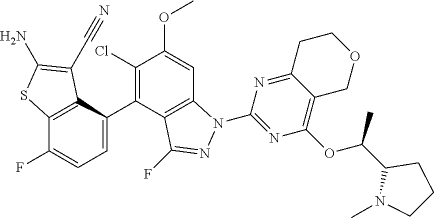 |
| 131 | 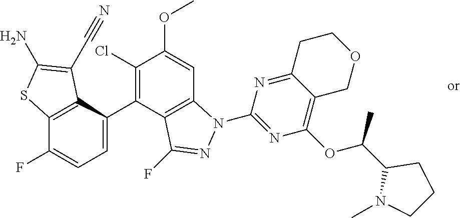 |
| | 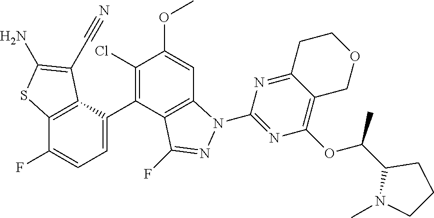 |
| 132 | 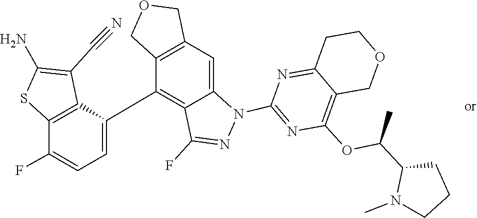 |
| | 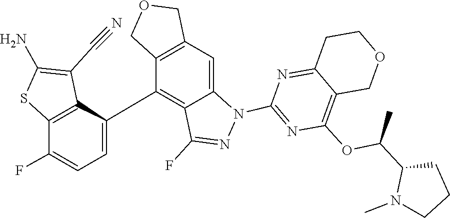 |
| 133 | 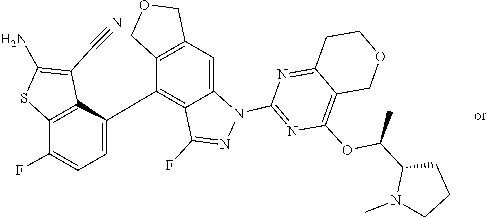 |
| | 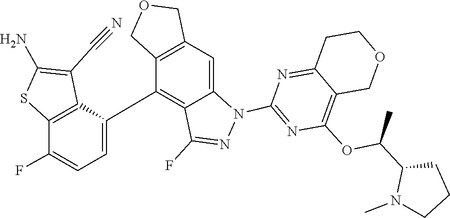 |
| 134 | 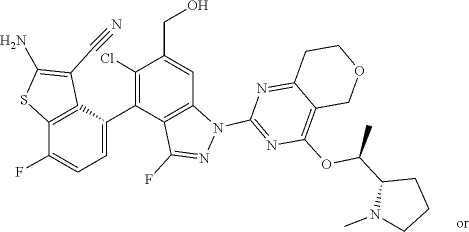 |
| | 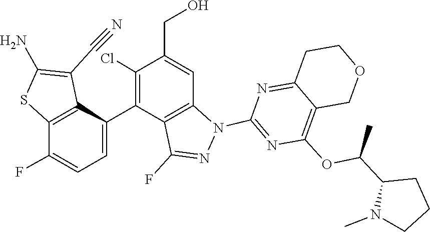 |
| 135 | 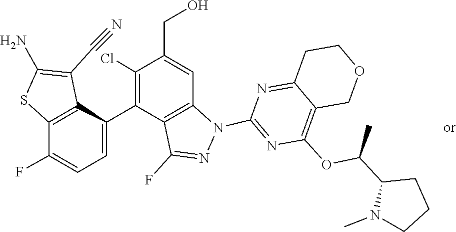 |
| | 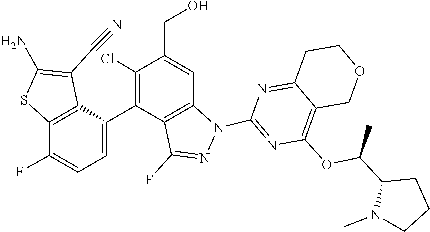 |
| 136 | 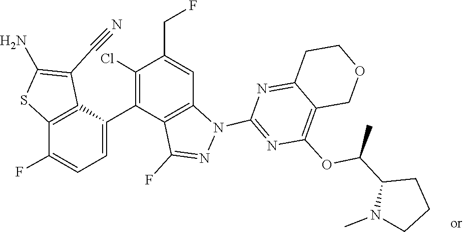 |
| | 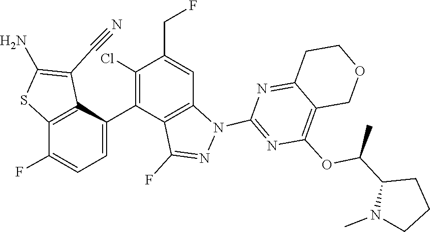 |
| 137 | 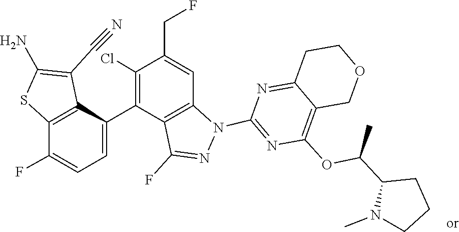 |
| | 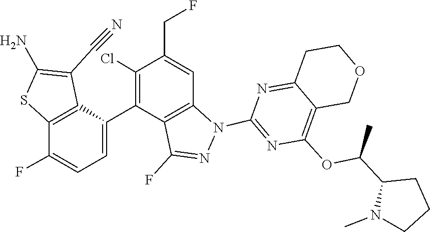 |
| 138 | 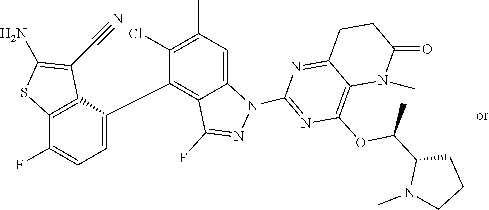 |
| | 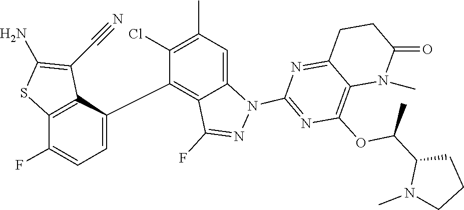 |
| 139 | 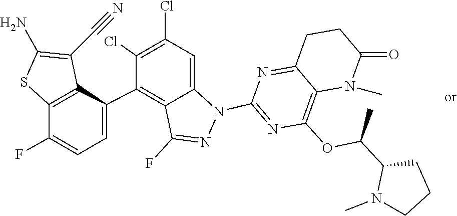 |
| | 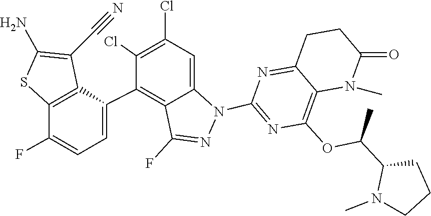 |
| 140 | 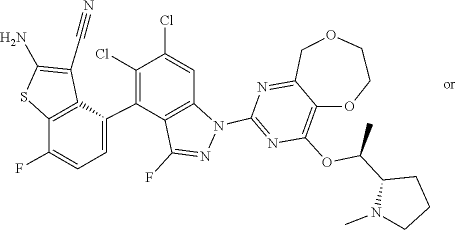 |
| | 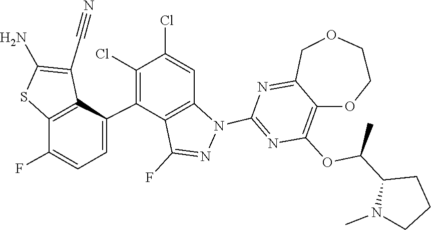 |
| 141 | 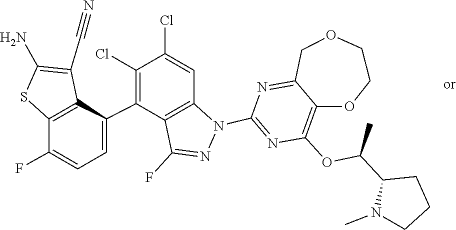 |
| | 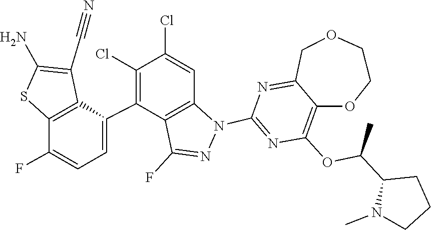 |
| 142 | 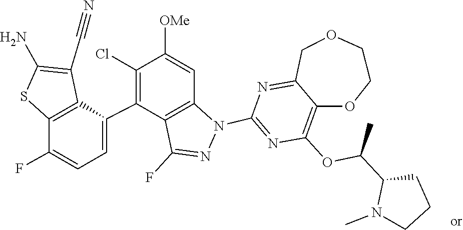 |
| | 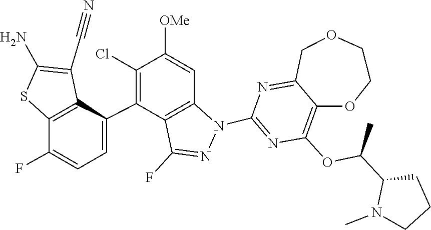 |
| 143 | 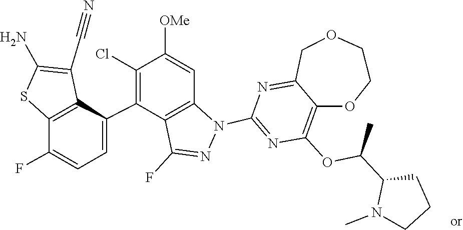 |
| | 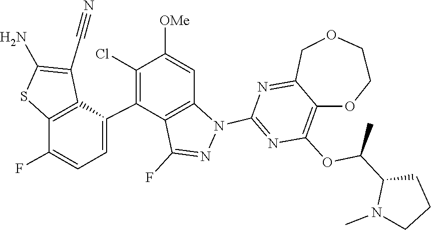 |
| 144 | 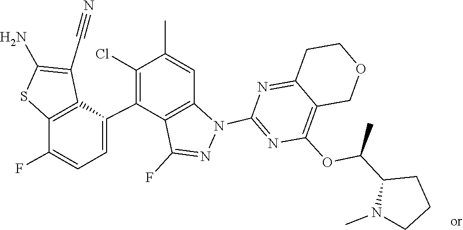 |
| | 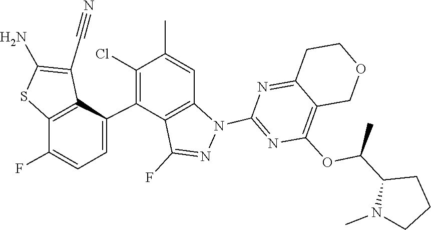 |
| 145 | 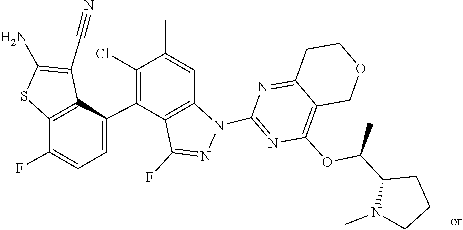 |
| | 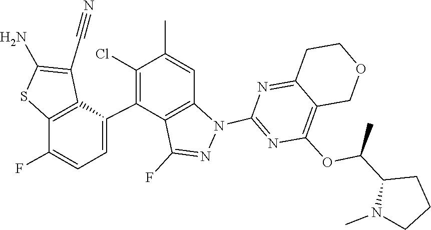 |
| 146 | 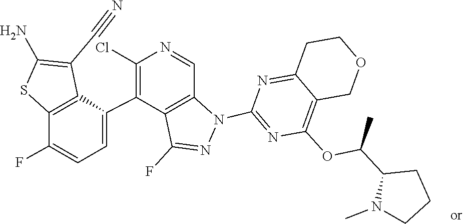 |
| | 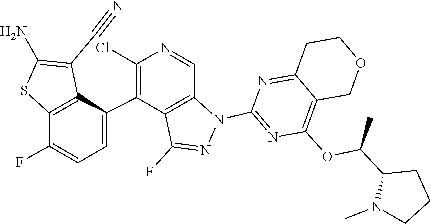 |
| 147 | 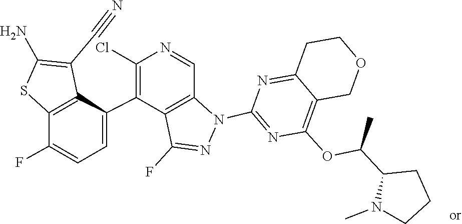 |
| | 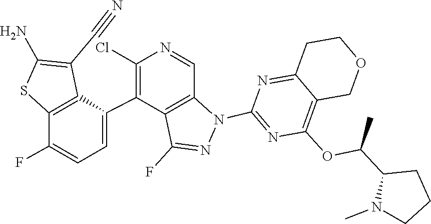 |
| 148 | 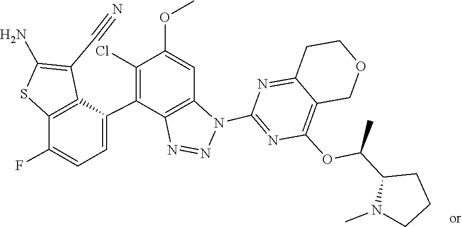 |
| | 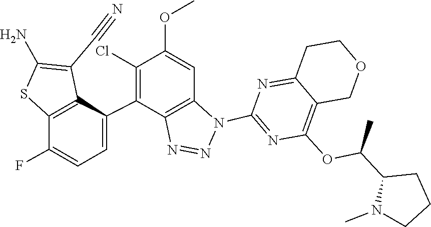 |
| 149 | 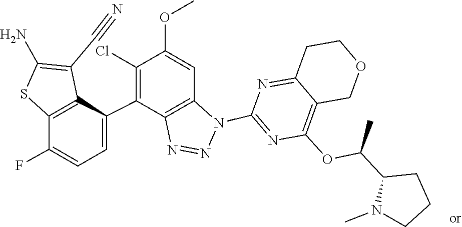 |
| | 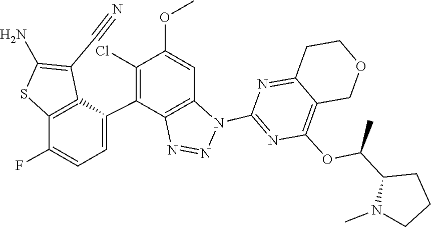 |
| 150 | 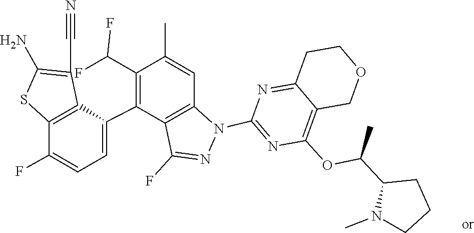 |
| | 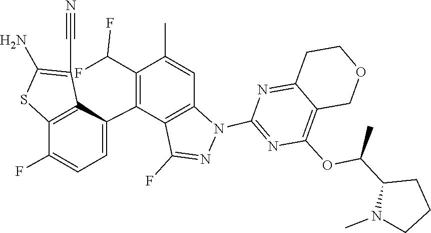 |
| 151 | 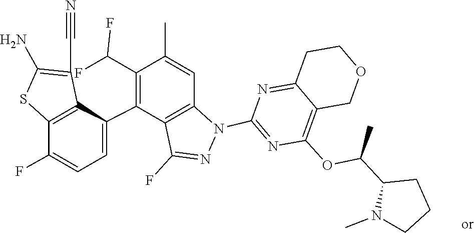 |
| | 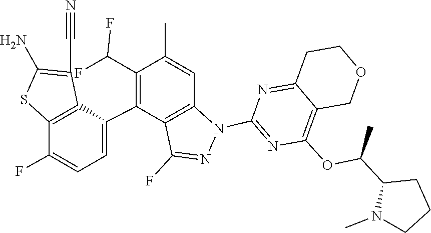 |
| 152 | 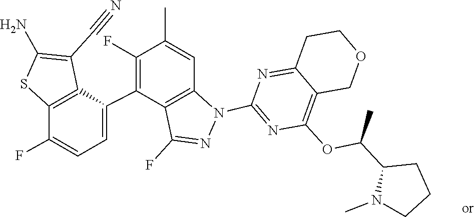 |
| | 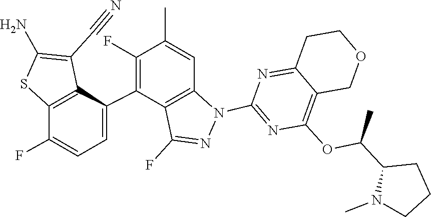 |
| 153 | 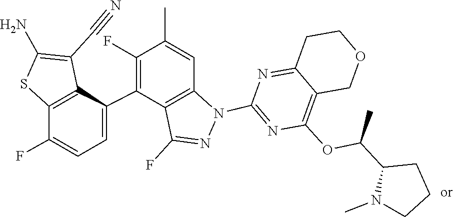 |
| | 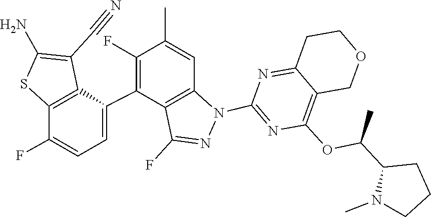 |
| 154 | 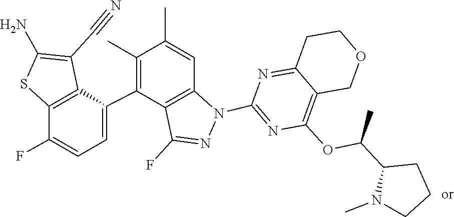 |
| | 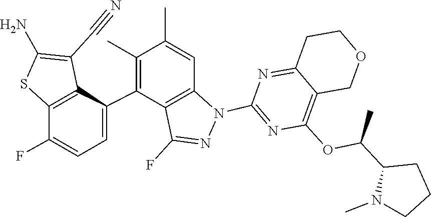 |
| 155 | 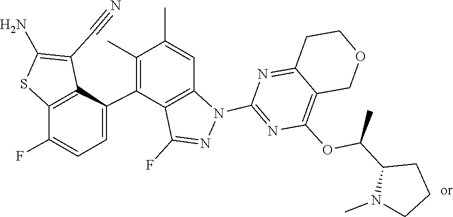 |
| | 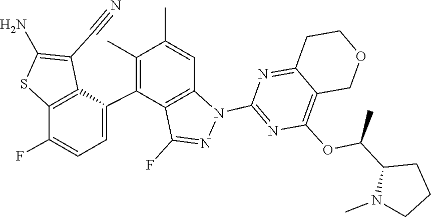 |
| 156 | 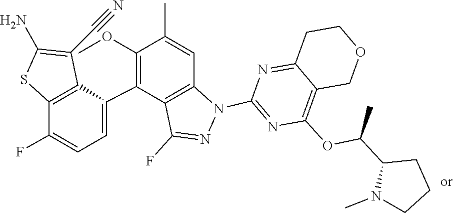 |
| | 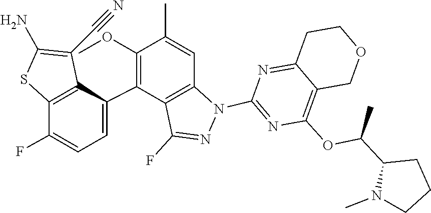 |
| 157 | 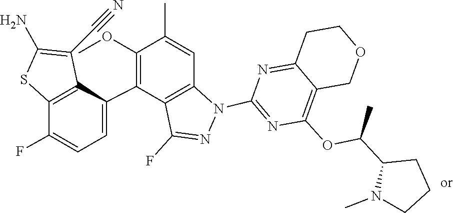 |
| | 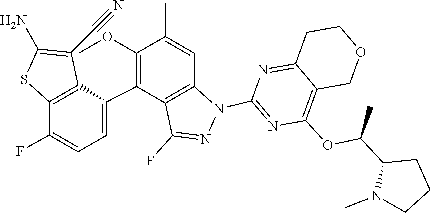 |
| 158 | 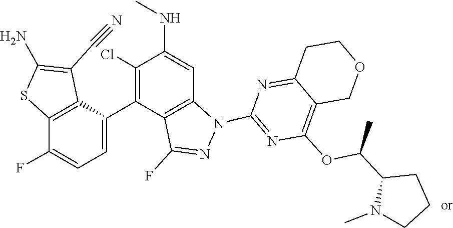 |
| | 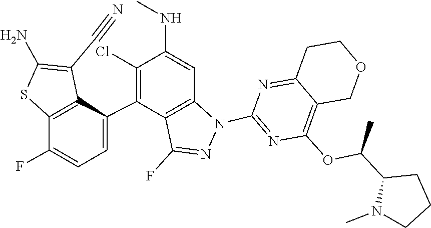 |
| 159 | 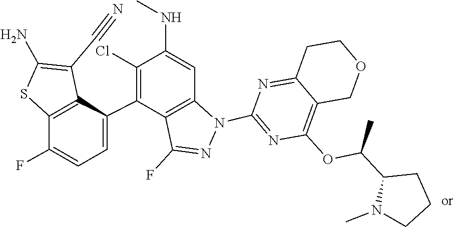 |
| | 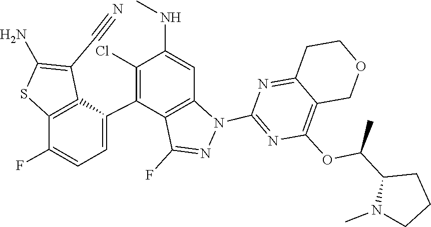 |
| 160 | 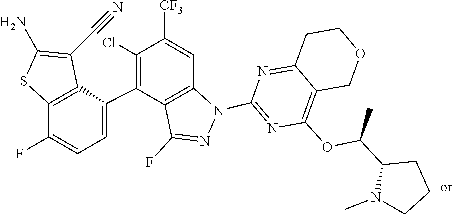 |
| | 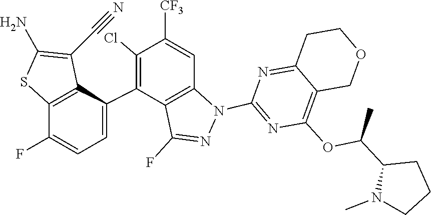 |
| 161 | 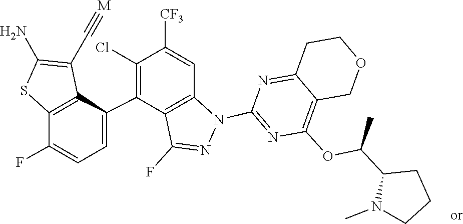 |
| | 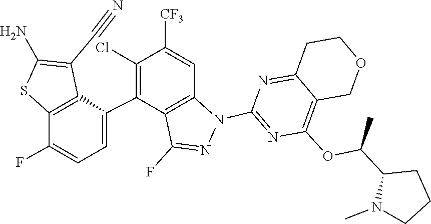 |
| 162 | 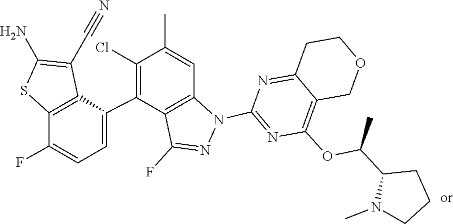 |
| | 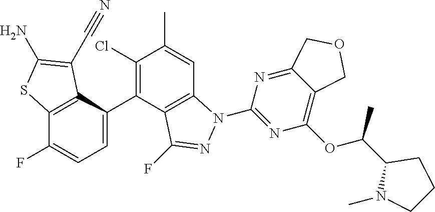 |
| 163 | 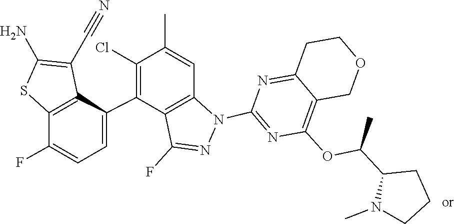 |
| | 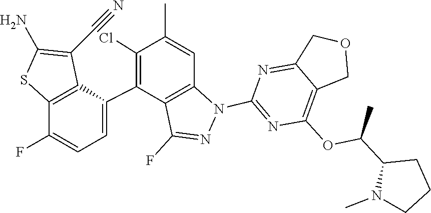 |
| 164 | 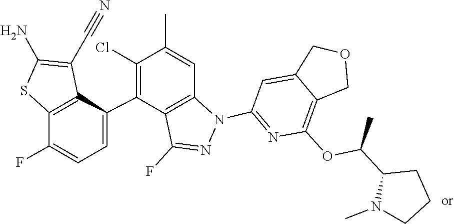 |
| | 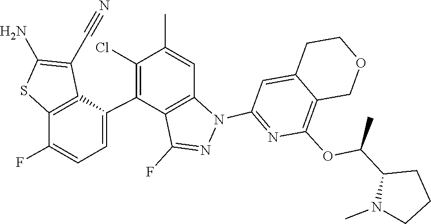 |
| 165 | 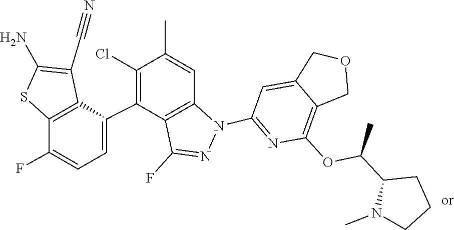 |
| | 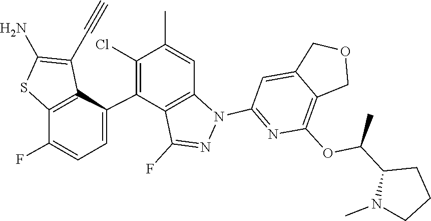 |
| 166 | 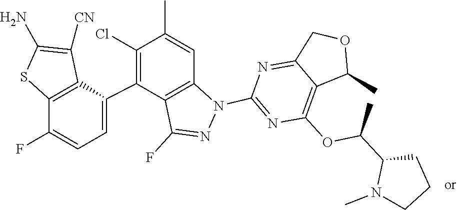 |
| | 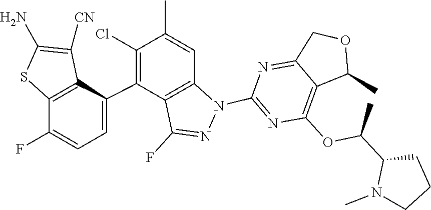 |
| | OR |
| | 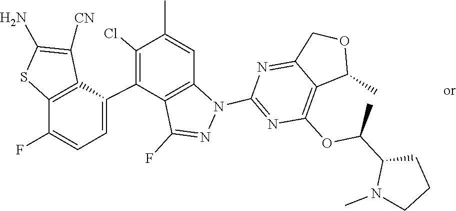 |
| | 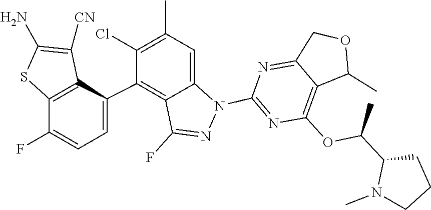 |
| 167 | 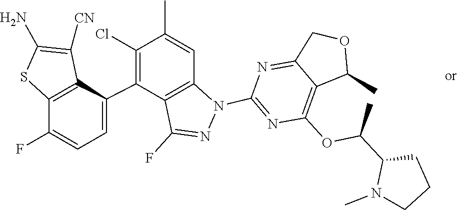 |
| | 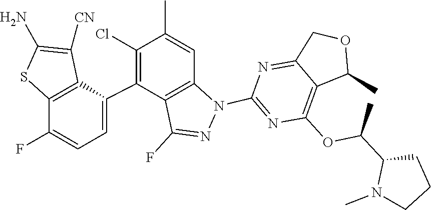 |
| | OR |
| | 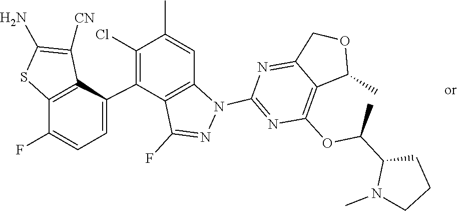 |
| | 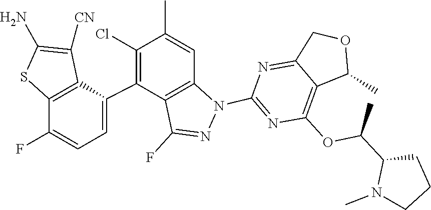 |
| 168 | 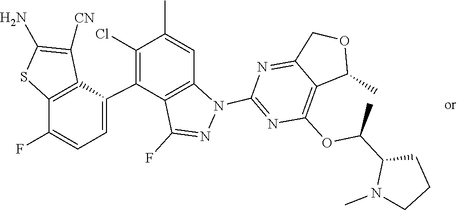 |
| | 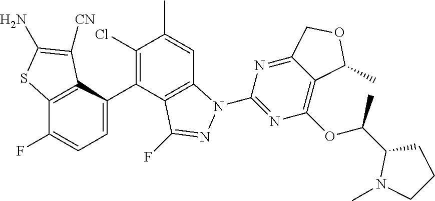 |
| | OR |
| | 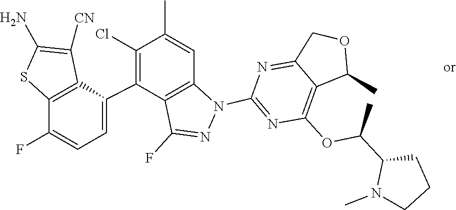 |
| | 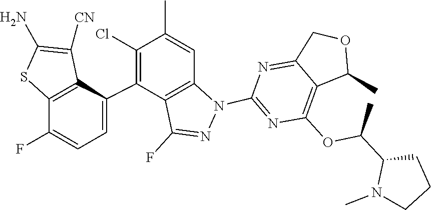 |
| 169 | 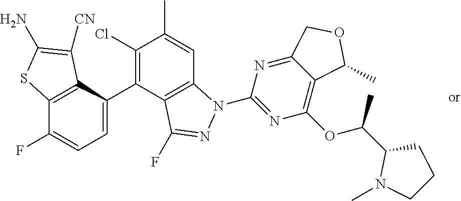 |
| | 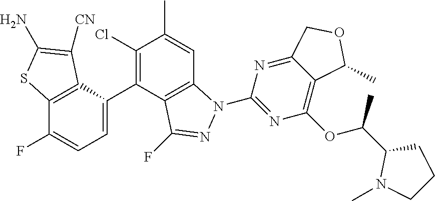 |
| | OR |
| | 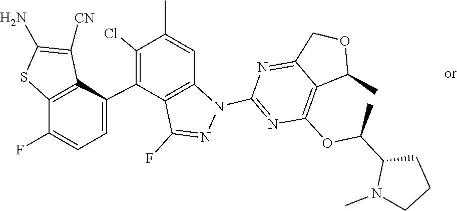 |
| | 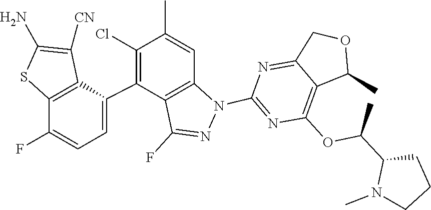 |
| 170 | 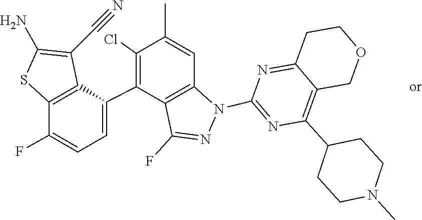 |
| | 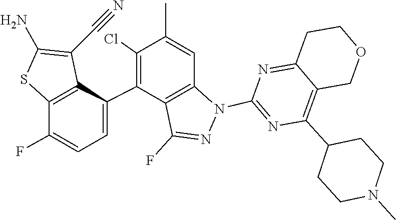 |
| 171 | 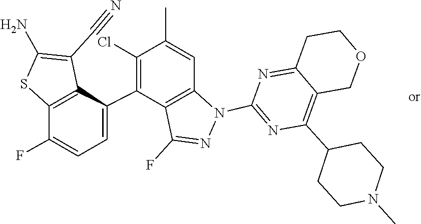 |
| | 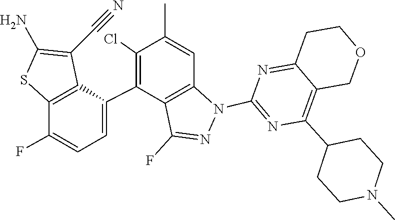 |
| 172 | 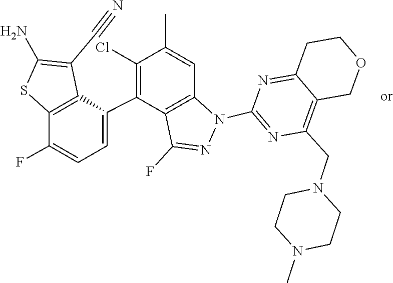 |
| | 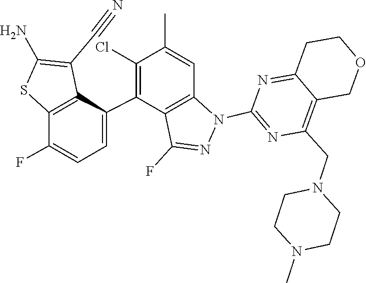 |
| 173 | 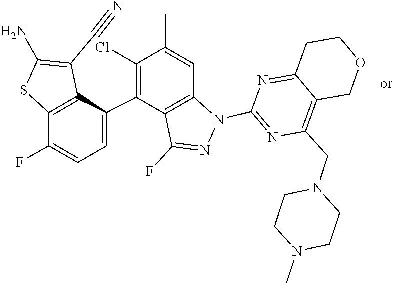 |
| | 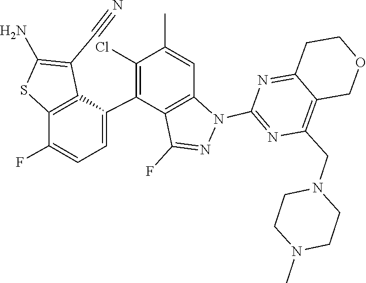 |

### Methods of Synthesis

By way of example and not in limitation, compounds of the disclosure and intermediate compounds can be prepared as outlined in Schemes 1-5, as well as in the Examples set forth herein from commercially available starting materials or known synthetic routes. The compounds employed as intermediates described in the Examples are included in the specification. It should be noted that one skilled in the art would know how to modify the procedures set forth in the illustrative schemes and Examples to arrive at the desired products. This includes the use of protecting groups: in some cases, certain substituents may interfere with the chemistry in these schemes, in which case appropriate protecting groups can be employed to avoid unwanted reactivity and side products.

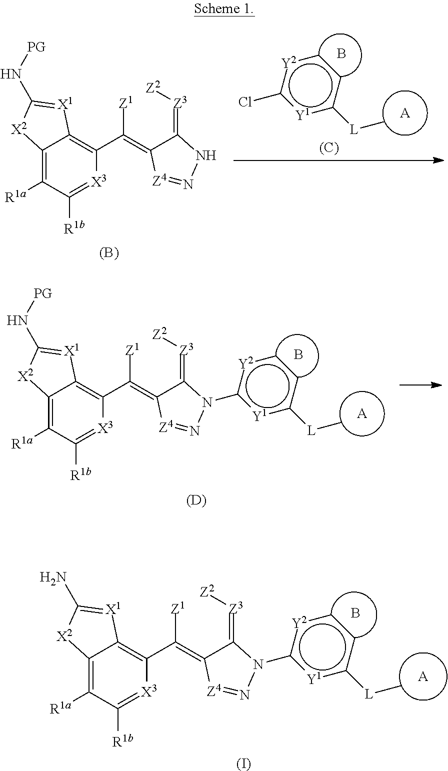

wherein PG is a suitable amine protecting group, preferably Boc. One skilled in the art would understand that the Cl group of Compound (C) can be replaced with other groups for alternative coupling reactions, e.g., methane sulfone or methyl sulfoxide.

As outlined in Scheme 1, compounds of Formula (I) can be prepared from a series of reactions from compound (B). Compound (D) can be obtained from a chloride (C) and compound (B) by a Buchwald-Hartwig type coupling reaction, using a suitable palladium catalyst in the presence of phosphine ligands, a suitable inorganic base, and a solvent such as dioxane or DCM at an elevated temperature. Alternatively, compound (D) can be obtained from a chloride, methyl sulfone or methyl sulfoxide (C) and compound (B) by an arylation reaction in the presence of a base in DMF at a temperature between ambient temperature and 100° C. Compound (I) can be obtained by deprotection of compound (D) under acidic conditions, preferably TFA in DCM or 6N HCl in dioxane at rt, or alternatively by reaction with NaI and TMSCl.

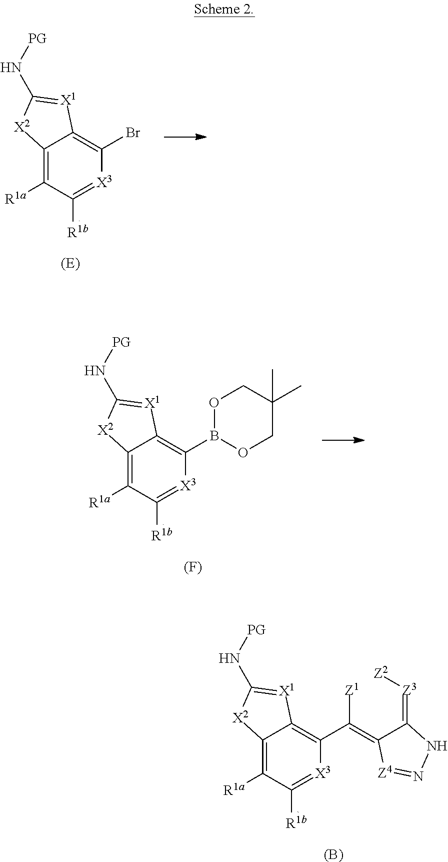

As outlined in Scheme 2, compound (B) can be prepared from a series of reactions from compound (E). Compound (F) can be obtained from a bromide (E) and 5,5,5′,5′-tetramethyl-2,2′-bi(1,3,2-dioxaborinane) in the presence of a base and a palladium catalyst in dioxane at about 95° C. Compound (B) can be obtained from compound (F) and a bromide (G) by a Suzuki-type palladium catalyzed cross coupling conditions.

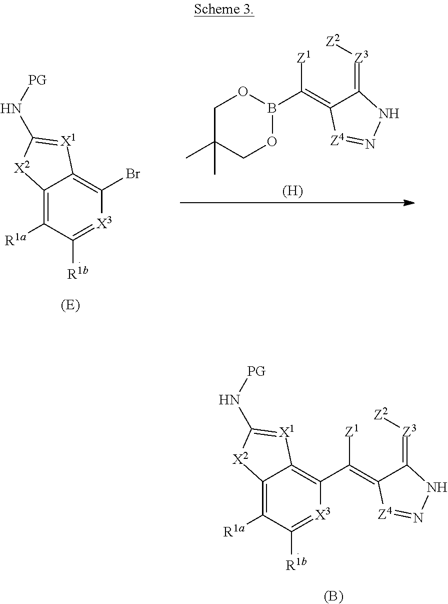

As outlined in Scheme 3, compound (B) can be prepared from a reaction from compound (E). Compound (B) can be obtained from compound (H) and a bromide (E) by Suzuki-type palladium catalyzed cross coupling conditions.

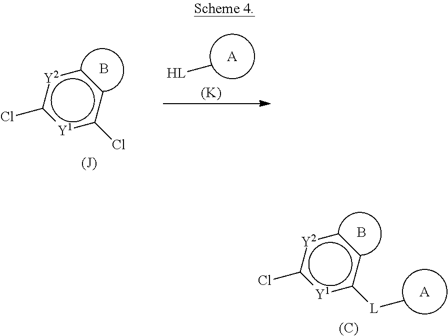

wherein L is —(CR^{La}R^{Lb})ₘ₁O(CR^{La}R^{Lb})ₘ₂— and m₁ is 0. One skilled in the art would understand that the Cl group of Compound (C) can be replaced with other groups for alternative coupling reactions, e.g., methane sulfone or methyl sulfoxide.

As outlined in Scheme 4, compound (C) can be prepared from a reaction from compound (J). Compound (C) can be obtained from compound (K) and a chloride (J) in the presence of an inorganic base in dioxane or toluene at a temperature between ambient temperature and about 80° C.

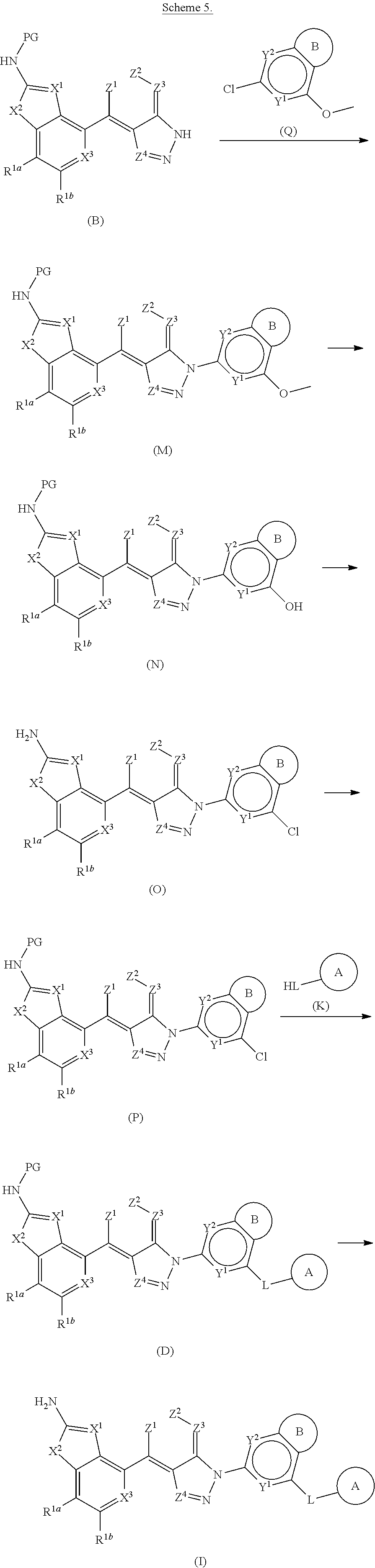

wherein L is —(CR^{La}R^{Lb})ₘ₁O(CR^{La}R^{Lb})ₘ₂— or —(CR^{La}R^{Lb})ₘ₁NR^{L}(CR^{La}R^{Lb})ₘ₂— and m1 is 0, or L is a bond and Ring A is attached to L through a N atom.

As outlined in Scheme 5, compounds of Formula (I) can be prepared from a series of reactions from compound (B). Compound (M) can be obtained from compound (B) and a chloride (Q) by a Buchwald-Hartwig type cross coupling reaction as previously described in Scheme 1. Compound (N) can be obtained from compound (M) by a de-methylation reaction using NaI and TMSCl. Compound (O) can be obtained by the chlorination of compound (N). Compound (P) can be obtained by the protection of compound (O), preferably, wherein PG is Boc. Compound (D) can be obtained from a chloride (P) and compound (K) as previously described in Scheme 4. Compound (I) can be obtained from compound (D) as previously described in Scheme 1.

Compounds of Formula (E), (F), (G), (H), (J), (K), and (Q), as described in Schemes 2-5, are commercially available or can be prepared by the methods described in the Intermediates and Examples below.

Compounds (I), (B), (C), (D), (E), (F), (G), (H), (M), (N), (O), (P), and (Q), as described in Schemes 1-5, can undergo further reaction to provide alternative compounds of formula (I), (B), (D), (E), (F), (G), (H), (M), (N), (O), (P), and (Q) by chemical transformations known to those skilled in the art. These transformations include, but are not limited to:
chlorination of an aromatic heterocycle
iodination of an aromatic heterocycle
reaction of a heteroaromatic halide with an alcohol to provide an ether
reaction of a heteroaromatic halide with an alkylzinc reagent or boronic acid or boranate to provide an alkyl substituted heteroaryl

It will be appreciated by those skilled in the art that it may be necessary to utilize a suitable protecting group strategy for the preparation of compounds of Formula (I). Typical protecting groups can comprise, a Boc or SEM group, for the protection of primary or secondary aliphatic amines, and BOC, SEM or THP for the protection of heteroaromatic N atoms.

If the chemical structure and chemical name conflict, the chemical structure is determinative of the identity of the compound.

The expressions “Peak 1” or “the first eluting compound” in the Examples section refer to a compound obtained from a chromatography separation/purification that elutes earlier than a second compound from the same reaction mixture. In some embodiments, the expressions “Peak 1” or “the first eluting isomer” in the Examples section refer to a stereoisomer of a compound of the disclosure obtained from a chromatography separation/purification that elutes earlier than a second stereoisomer of a compound of the disclosure from the same reaction mixture. The second compound is referred to as “Peak 2” or “the second eluting compound”. In some embodiments, the second stereoisomer of a compound of the disclosure is referred to as “Peak 2” or “the second eluting isomer”.

The use of stereoisomerically pure forms of the compounds of the disclosure, as well as the use of mixtures of those forms, are encompassed by the embodiments herein. For example, mixtures comprising equal or unequal amounts of the enantiomers or atropisomers of a particular compound of the disclosure can be used in the methods and compositions disclosed herein. These isomers can be asymmetrically synthesized or resolved using standard techniques such as chiral columns (e.g., chiral SFC, chiral-phase gas chromatography, or chiral-phase high performance liquid chromatography) or chiral resolving agents (e.g., crystallizing the compound in a chiral solvent), e.g., as indicated in the Examples below. Enantiomers, diastereomers, and atropisomers can also be obtained from enantiomerically-pure, diastereomerically-pure, or atropisomeric-pure intermediates, reagents, and catalysts by known synthetic methods.

The term “stereoisomers” refers to compounds that differ only in their spatial arrangement and includes all geometric isomers (E/Z), diastereomeric forms, enantiomeric forms, and atropoisomeric forms of a compound of the disclosure. Enantiomers are stereoisomers that are mirror images of each other. Diastereomers are stereoisomers having two or more chiral centers that are not superimposable and are not mirror images of each other. Atropisomers are chiral compounds in which stereoisomers arise due to hindered rotation about a single bond, where energy differences due to steric strain or other contributors create a barrier to rotation that is high enough to allow for isolation of individual conformers. A racemic mixture means a mixture of 50% of one enantiomer (or atropisomer) and 50% of its corresponding enantiomer (or atropisomer).

Compounds having one or more chiral centers may exist in various stereoisomeric forms, i.e., each chiral center may have an R or S configuration, or may be a mixture of both. The disclosure encompasses all enantiomerically-pure, enantiomerically-enriched, racemic mixtures, diastereomerically-pure, diastereomerically-enriched, diastereomeric mixtures, atropisomerically-pure, atropisomerically-enriched, atropisomeric mixtures of the compounds.

When the stereochemical configuration at a chiral center in a compound of the disclosure having one or more chiral centers is depicted by its chemical name (e.g., where the configuration is indicated in the chemical name by “R” or “S”) or structure (e.g., where the configuration is indicated by “wedge” bonds) that indicates a single enantiomer or a single atropisomer, the compound of the disclosure is at least 50%, 60%, 70%, 75%, 80%, 85%, 90%, 95%, 98%, 99% or 99.9% optically pure (also referred to as “enantiomerically-pure” or “atropisomerically-pure”). Optical purity is the weight in the mixture of the named or depicted enantiomer or atropisomer divided by the total weight in the mixture of both enantiomers or atropisomers.

When two stereoisomers are depicted by their chemical names or structures, and the names or structures are connected by an “or”, one or the other of the two stereoisomers is intended, but not both. When two stereoisomers are depicted by their chemical names or structures, and the names or structures are connected by an “and” or “+”, both stereoisomers have been prepared as described in the Examples section.

When a compound of the disclosure having a chiral center is depicted by a structure without showing a configuration at that chiral center, the structure is meant to encompass the compound with the S configuration at that chiral center, the compound with the R configuration at that chiral center, or the compound with a mixture of the R and S configuration at that chiral center. When a compound of the disclosure having a chiral center is depicted by its chemical name without indicating a configuration at that chiral center with “S” or “R”, the name is meant to encompass the compound with the S configuration at that chiral center, the compound with the R configuration at that chiral center or the compound with a mixture of the R and S configuration at that chiral center.

The term “tautomers” refers to structural isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

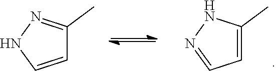

As readily understood by one skilled in the art, a wide variety of functional groups and other structures may exhibit tautomerism. Examples of proton tautomerization may occur in compounds containing an imino/amino, keto/enol, oxime/nitroso radical, or lactam/lactim radicals. The individual tautomers as well as mixtures thereof are encompassed within the compounds of the disclosure.

In the compounds of the disclosure, any position specifically designated as “D” or “deuterium” is understood to have deuterium enrichment at least 5, 10, 25, 50, 80, 90, 95, 98 or 99%. When a position is designated as “H” or “hydrogen”, the position has hydrogen at its natural abundance. When a position is silent as to whether hydrogen or deuterium is present, the position has hydrogen at its natural abundance. The expression “deuterium enrichment” refers to the percentage of enrichment at any one of the sites where hydrogen has been replaced by deuterium by at least 50%, 60%, 70%, 75%, 80%, 85%, 90%, 95%, 98%, 99%, or 99.9%. Deuterium enrichment is a mole percent and is obtained by dividing the number of compounds with deuterium enrichment at the site of enrichment with the number of compounds having hydrogen or deuterium at the site of enrichment.

The expression “isotopically enriched” refers to a compound containing one or more atoms are replaced by atoms having the same atomic number, but an atomic mass or mass number different from the atomic mass or mass number usually found in nature. The compounds of the disclosure may be isotopically enriched at one or more of the atoms. The compounds of the disclosure may be radiolabeled with radioactive isotopes, such as for example tritium (³H), iodine-125 (¹²⁵I), sulfur-35 (³⁵S), or carbon-14 (¹⁴C), or may be isotopically enriched, such as with deuterium (²H), carbon-13 (¹³C), or nitrogen-15 (¹⁵N). Radiolabeled and isotopically enriched compounds are useful as therapeutic agents, e.g., cancer therapeutic agents, research reagents, e.g., binding assay reagents, and diagnostic agents, e.g., in vivo imaging agents. All isotopic variations of the compounds of the disclosure, whether radioactive or not, are intended to be encompassed within the scope of the embodiments.

### Pharmaceutical Compositions

The disclosure also relates to a pharmaceutical composition comprising a compound of the disclosure or a pharmaceutically acceptable salt of a compound of the disclosure and a pharmaceutically acceptable excipient.

A pharmaceutical composition may comprise a compound of the disclosure or a pharmaceutically acceptable salt thereof and a pharmaceutically acceptable excipient, such as carriers, diluents, binders, fillers, disintegrants, lubricants, solubilizers, coatings, sweeteners, flavors, and colors, and the like. Such preparations can be sterilized and, if desired, mixed with one or more auxiliary agents such as lubricants, preservatives, stabilizers, wetting agents, emulsifiers, salts for influencing osmotic pressure, buffers, coloring, and aromatic substances and the like that do not deleteriously react with or interfere with the activity of the compounds of the disclosure. In some embodiments, the pharmaceutically acceptable excipient has one or more of the following effects: aiding the formulation of the compounds of the disclosure, aiding the administration of a compound of the disclosure to a patient, aiding absorption of a compound of the disclosure by a patient, and facilitating manufacture of the dosage form. More complete listings of suitable excipients may be found in the Handbook of Pharmaceutical Excipients (9^{th} Ed., Pharmaceutical Press (2020)). Conventional procedures and ingredients for the selection and preparation of suitable formulations are described, for example, in Remington: The Science and Practice of Pharmacy (23^{rd} Ed., Academic Press (2020)) and in The United States Pharmacopeia: The National Formulary (USP-NF) published in 2024.

The expression “effective amount” refers to an amount of a compound or a pharmaceutically acceptable salt thereof sufficient to elicit the desired biological response in a patient in need thereof. The precise amount of compound or pharmaceutically acceptable salt thereof administered to provide an “effective amount” to the patient will depend on the activity of the particular compound employed, the metabolic stability of the compound, the mode of administration, the time of administration, the duration of the treatment, other drugs, compounds and/or materials used in combination with the particular active ingredient employed, the type and severity of the disease, the rate of excretion of the particular active ingredient being employed, and on the characteristics of the patient, such as the age, sex, body weight, general health and prior medical history of the patient being treated, tolerance to drugs, and like factors known in the medical arts. For example, an effective amount may be about 0.01 mg/kg to about 100 mg/kg per day.

In general, a suitable dose of a compound of the disclosure will be that amount of the compound of the disclosure that is effective to produce a therapeutic effect and will depend upon the factors described above.

### Methods of Treatment

The disclosure relates to a method of treating a disease or condition mediated by KRAS, comprising providing to a patient in need thereof any of the compounds of the disclosure or a pharmaceutically acceptable salt thereof. The method comprises administrating to the patient an effective amount of a compound of the disclosure or a pharmaceutically acceptable salt thereof, or a pharmaceutical composition of the disclosure.

The disclosure also relates to a method of inhibiting KRAS in a patient in need thereof, comprising administrating to the patient an effective amount of a compound of the disclosure or a pharmaceutically acceptable salt thereof, or a pharmaceutical composition of the disclosure. The inhibition of KRAS may be assessed and demonstrated by a wide variety of ways known in the art. Kits and commercially available assays may be utilized for determining whether and to what degree KRAS has been inhibited.

The disclosure also relates to a use of a compound of the disclosure or a pharmaceutically acceptable salt thereof, or a pharmaceutical composition of the disclosure in the manufacture of a medicament.

The disclosure also relates to a use of a compound of the disclosure or a pharmaceutically acceptable salt thereof, or a pharmaceutical composition of the disclosure in the manufacture of a medicament for the treatment of a disease or condition mediated by KRAS.

The disclosure also relates to a compound of the disclosure or a pharmaceutically acceptable salt thereof, or a pharmaceutical composition of the disclosure for use as a drug.

The disclosure also relates to a compound of the disclosure or a pharmaceutically acceptable salt thereof, or a pharmaceutical composition of the disclosure for use in treating a disease or condition mediated by KRAS.

The disclosure also relates to a use of a compound of the disclosure or a pharmaceutically acceptable salt thereof for use as a drug.

The disclosure also relates to a use of a compound of the disclosure or a pharmaceutically acceptable salt thereof for the treatment of a disease or condition mediated by KRAS.

In addition to embodiments in the preceding paragraphs, the methods and uses include those in which the effect is one or more of:
(A) partially or substantially reducing the extent of the disease or condition;
(B) partially or substantially ameliorating or improving a clinical symptom or indicator associated with the disease or condition;
(C) partially or substantially stabilizing the disease or condition;
(D) partially or substantially delaying, inhibiting or decreasing the likelihood of the progression of the disease or condition;
(E) partially or substantially decreasing the likelihood of recurrence of the disease or condition;
(F) partially or substantially prolonging the survival of a patient with the disease or condition;
(G) providing partial or total remission of the disease or condition;
(H) enhancing the effect of another medication used to treat the disease or condition;
(I) increasing the quality of life of the patient having the disease or condition;
wherein the disease or condition is mediated by KRAS activity, for example, by KRAS alterations such as KRAS mutations, or by one or more of amplification, overexpression, and activation of KRAS wildtype.

In some embodiments, the disease or condition is mediated by KRAS, for example, by KRAS alterations such as KRAS mutations, or by amplification, overexpression, and/or activation of KRAS wildtype. In some embodiments, the disease or condition is cancer. In some embodiments, the cancer comprises one or more KRAS mutation or two or more combinations thereof. In some embodiments, the cancer comprises a mutation in a receptor tyrosine kinase (RTK), including EGFR, ERBB2, FGFR, and MET mutations, in addition to the KRAS mutation. In some embodiments, the disease is a developmental disease or syndrome mediated by alterations in one or more genes in the KRAS/MAPK pathway, for example, by KRAS mutations, or by mutation of other genes in the KRAS/MAPK pathway that leads to activation of KRAS signaling.

In some embodiments, the compounds disclosed herein are not potent ligands for PXR (e.g., <25% induction relative to rifampicin), and as such drug-drug interactions (DDI) related to pregnane X receptor (PXR) activation are less likely to occur for those compounds. The PXR is a nuclear hormone receptor responsible for the upregulation of enzymes and transporters involved in metabolism and clearance, including cytochrome P450 3A4 (CYP 3A4). PXR is known to bind to a wide variety of drug-like molecules, and following ligand binding, PXR can initiate the transcription of genes related to xenobiotic metabolism and transport, which ultimately allows for translation into active proteins. PXR activation thus affects the pharmacokinetics of any other drug that is metabolized and/or transported via the proteins that PXR binding upregulates. The PXR ligand is labeled a DDI perpetrator, and it is less favorable to co-administer such a compound with other drugs since this will decrease the half-life of these other drugs, potentially leading to a decrease in their efficacy. See, for example, The Pregnane X Receptor: From Bench to Bedside. Expert Opin Drug Metab Toxicol. 2008, 4, 895-908. Assays for PXR activation are commercially available or known to those of skill in the art.

Described herein is a method of treating a disease or disorder mediated by KRAS, comprising providing to a subject in need thereof any of the compounds disclosed in this application or a pharmaceutically acceptable salt thereof.

In some embodiments, the KRAS is KRAS wild type (KRAS^{WT}).

In some embodiments, the KRAS^{WT} is amplified, overexpressed, or activated.

In some embodiments, the KRAS comprises a mutation.

In some embodiments, the disease or disorder is mediated by two or more types of KRAS variants.

In some embodiments, the KRAS mutation is selected from KRAS^{G12A}, KRAS^{G12C}, KRAS^{G12D}, KRAS^{G12R}, KRAS^{G12S}, KRAS^{G12V}, KRAS^{G13A}, KRAS^{G13C}, KRAS^{G13D}, KRAS^{G13R}, KRAS^{G12S}, KRAS^{G13V}, KRAS^{Q61E}, KRAS^{Q61H}, KRAS^{Q61K}, KRAS^{Q61L}, KRAS^{Q61P}, KRAS^{Q61R}, KRAS^{A146P}, KRAS^{A146T}, and KRAS^{A146V}.

In some embodiments, the KRAS mutation is selected from KRAS^{G12D}, KRAS^{G12V} KRAS^{G12C}, KRAS^{G12A}, KRAS^{G12R}, and KRAS^{G13D}. In other embodiments, the KRAS mutation is selected from KRAS^{G12C}, KRAS^{G12V}, KRAS^{G12D}, KRAS^{G12A}, KRAS^{G12S}, and KRAS^{G13D}.

In some embodiments, the disease or disorder is cancer.

In some embodiments, the cancer is selected from pancreatic adenocarcinoma, colorectal adenocarcinoma, non-small cell lung carcinoma (e.g., squamous cell carcinoma and adenocarcinoma of the lung), prostate adenocarcinoma, uterine endometrial carcinoma, cholangiocarcinoma, testicular cancer, cervical squamous cell carcinoma, appendiceal cancer, myelodysplastic syndrome, acute myeloid leukemia (AML), juvenile myelomonocytic leukemia, ovarian adenocarcinoma, gastric adenocarcinoma, esophageal adenocarcinoma, small bowel adenocarcinoma, appendiceal carcinoma, and breast adenocarcinoma. In a particular embodiment, the cancer is selected from pancreatic adenocarcinoma, colorectal adenocarcinoma, and non-small cell lung carcinoma (e.g., squamous cell carcinoma and adenocarcinoma of the lung).

In some embodiments, the disease or disorder is a developmental disease or syndrome.

In some embodiments, the compound for administration or use is selected from Table 1.

Compounds of Formula (I), or a pharmaceutically acceptable salt thereof, and pharmaceutical compositions comprising compounds of Formula (I), or a pharmaceutically acceptable salt thereof, can be used in methods of treating a disease or disorder mediated by KRAS, for example, by KRAS alterations such as KRAS mutations, or by amplification, overexpression and/or activation of KRAS wildtype. The types of KRAS targets include, but are not limited to, KRAS G12A, KRAS G12C, KRAS G12D, KRAS G12R, KRAS G12S, KRAS G12V, KRAS G13A, KRAS G13C, KRAS G13D, KRAS G13R, KRAS G12S, KRAS G13V, KRAS Q61E, KRAS Q61H, KRAS Q61K, KRAS Q61L, KRAS Q61P, KRAS Q61R, KRAS A146P, KRAS A146T, KRAS A146V mutations. In some embodiments, the disease or condition is cancer. In some embodiments, the cancer comprises a KRAS mutation or two or more combinations thereof. In some embodiments, the cancer cells can comprise not only KRAS mutations and/or amplification/overexpression/activation of KRAS wildtype, but also a mutation in a receptor tyrosine kinase (RTK), including EGFR, ERBB2, FGFR, and MET mutations. The compounds and pharmaceutical compositions can also be used in in vitro methods, such as in vitro methods of administering a compound or pharmaceutical composition to cells to inhibit KRAS, for example, KRAS mutations. The types of KRAS targets include, but are not limited to, KRAS G12A, KRAS G12C, KRAS G12D, KRAS G12R, KRAS G12S, KRAS G12V, KRAS G13A, KRAS G13C, KRAS G13D, KRAS G13R, KRAS G12S, KRAS G13V, KRAS Q61E, KRAS Q61H, KRAS Q61K, KRAS Q61L, KRAS Q61P, KRAS Q61R, KRAS A146P, KRAS A146T, KRAS A146V mutations. The KRAS target can present one or more of these mutations. The wildtype KRAS is also a target, for example, wildtype KRAS that is amplified, overexpressed, and/or activated.

In one aspect, provided herein is a method of inhibiting KRAS, for example, KRAS mutations, comprising contacting either an effective amount of a compound described herein, or a pharmaceutically acceptable salt thereof, or an effective amount of a pharmaceutical composition provided herein, with KRAS. The types of KRAS targets include, but are not limited to, KRAS G12A, KRAS G12C, KRAS G12D, KRAS G12R, KRAS G12S, KRAS G12V, KRAS G13A, KRAS G13C, KRAS G13D, KRAS G13R, KRAS G12S, KRAS G13V, KRAS Q61E, KRAS Q61H, KRAS Q61K, KRAS Q61L, KRAS Q61P, KRAS Q61R, KRAS A146P, KRAS A146T, KRAS A146V mutations. The wildtype KRAS is also a target, for example, wildtype KRAS that is amplified, overexpressed, and/or activated. The inhibition of KRAS can be assessed and demonstrated by a wide variety of ways known in the art. Kits and commercially available assays can be utilized for determining whether and to what degree KRAS has been inhibited. In certain embodiments, the compounds of the present disclosure are pan-KRAS inhibitors. In some embodiments, the pan-KRAS inhibitors of the present disclosure inhibit a KRAS mutation or two or more combinations thereof.

In some embodiments, provided herein is a method of inhibiting KRAS comprising contacting either an effective amount of a compound described herein, or a pharmaceutically acceptable salt thereof, or an effective amount of a pharmaceutical composition provided herein, with KRAS. In some embodiments, the KRAS is amplified, or overexpressed, and/or activated KRAS wildtype. In some embodiments, the KRAS is a KRAS mutant. In certain embodiments, the compounds of the present disclosure are pan-KRAS inhibitors. In some embodiments, the pan-KRAS inhibitors of the present disclosure inhibit a KRAS mutation or two or more combinations thereof.

In another aspect, provided herein is a method for treating a disease mediated by KRAS, for example, by KRAS alterations such as KRAS mutations, or by amplification, overexpression, and/or activation of KRAS wildtype, in a subject in need thereof, comprising administering to the subject an effective amount of a compound of Formula (I) or a pharmaceutically acceptable salt thereof. In some embodiments, the disease mediated by KRAS, for example, by KRAS alterations such as KRAS mutations, or by amplification, overexpression and/or activation of KRAS wildtype, is cancer. Non-limiting examples of a cancer mediated by KRAS, for example, by KRAS alterations such as KRAS mutations, or by amplification, overexpression, and/or activation of KRAS wildtype, include lung cancer, mediastinum cancer, gastrointestinal (GI) tract cancer, testis cancer, gynecological cancer, breast cancer, endocrine system cancer, soft tissue sarcoma, bone sarcoma, mesothelioma, skin cancer, neoplasms of the central and peripheral nervous systems, lymphomas, leukemias, and head and neck cancers.

In some embodiments, cancer mediated by KRAS, for example, by KRAS alterations such as KRAS mutations, or by amplification, overexpression, and/or activation of KRAS wildtype is lung cancer. Non-limiting examples of lung cancer include non-small cell lung cancer (NSCLC) (e.g., squamous cell carcinoma, spindle cell carcinoma, adenocarcinoma, large cell carcinoma, clear cell carcinoma, and bronchioalveolar) and small cell lung cancer (SCLC) (e.g., oat cell cancer, intermediate cell cancer, and combined oat cell cancer).

In some embodiments, cancer mediated by KRAS, for example, by KRAS alterations such as KRAS mutations, or by amplification, overexpression, and/or activation of KRAS wildtype is mediastinum cancer. Non-limiting examples of mediastinum cancer include neurogenic tumors (e.g., including neurofibroma, neurilemoma, malignant schwannoma, neurosarcoma, ganglioneuroblastoma, ganglioneuroma, neuroblastoma, pheochromocytoma, and paraganglioma), germ cell tumors (e.g., seminoma, teratoma, and non-seminoma), thymic tumors (e.g., thymoma, thymolipoma, thymic carcinoma, and thymic carcinoid), mesenchymal tumors (e.g., fibroma, fibrosarcoma, lipoma, liposarcoma, myxoma, mesothelioma, leiomyoma, leiomyosarcoma, rhabdomyosarcoma, xanthogranuloma, mesenchymoma, hemangioma, hemangioendothelioma, hemangiopericytoma, lymphangioma, lymphangiopericytoma, and lymphangiomyoma).

In some embodiments, cancer mediated by KRAS, for example, by KRAS alterations such as KRAS mutations, or by amplification, overexpression, and/or activation of KRAS wildtype is gastrointestinal (GI) tract cancer. Non-limiting examples of gastrointestinal (GI) tract cancer include esophagus cancer, stomach cancer (gastric cancer), gastroesophageal junction cancer, pancreas cancer (including pancreatic adenocarcinoma), liver and biliary tree cancer (e.g., hepatocellular carcinoma (HCC) such as childhood HCC, fibrolamellar HCC, combined HCC, spindle cell HCC, clear cell HCC, giant cell HCC, carcinosarcoma HCC, sclerosing and HCC; hepatoblastoma; cholangiocarcinoma; cholangiocellular carcinoma; hepatic cystadenocarcinoma; angiosarcoma; hemangioendothelioma; leiomyosarcoma; malignant schwannoma; fibrosarcoma; and Klatskin tumor), gall bladder cancer, extrahepatic bile ducts cancer (including ampullary cancer), small intestine cancer (e.g., duodenum, jejunum, and ileum), appendiceal cancer, large intestine cancer (e.g., cecum, colon, rectum, anus, colorectal cancer (including colorectal adenocarcinoma), and gastrointestinal stroma tumor (GIST)), genitourinary system cancer (e.g., kidney cancer such as renal pelvis, renal cell carcinoma (RCC), nephroblastoma (Wilms' tumor), hypernephroma, Grawitz tumor; ureter; urinary bladder such as urachal cancer, and urothelial cancer; urethra such as distal, bulbomembranous, and prostatic (including prostate adenocarcinoma); prostate such as androgen dependent, androgen independent, castration resistant, hormone independent, and hormone refractory), and penis), and gastric cancer.

In some embodiments, cancer mediated by KRAS, for example, by KRAS alterations such as KRAS mutations, or by amplification, overexpression, and/or activation of KRAS wildtype is a reproductive system cancer including testis cancer (or testicular cancer) and gynecologic cancer. Non-limiting examples of testis cancer include seminomas and non-seminomas. Non-limiting examples of gynecologic cancer include cancers of the ovary, fallopian tube, peritoneum, cervix (including cervical squamous cell carcinoma), vulva, vagina, and uterine body, including of the endometrium (such as endometrial carcinoma) and fundus.

In some embodiments, cancer mediated by KRAS, for example, by KRAS alterations such as KRAS mutations, or by amplification, overexpression, and/or activation of KRAS wildtype is breast cancer. Non-limiting examples of breast cancer include mammary carcinoma (e.g., infiltrating ductal, colloid, lobular invasive, tubular, adenocystic, papillary, medullary, and mucinous), hormone receptor positive breast cancer (e.g., estrogen receptor positive breast cancer, and progesterone receptor positive breast cancer), Her2 positive breast cancer, triple negative breast cancer, and Paget's disease of the breast.

In some embodiments, cancer mediated by KRAS, for example, by KRAS alterations such as KRAS mutations, or by amplification, overexpression, and/or activation of KRAS wildtype is endocrine system cancer. Non-limiting examples of endocrine system cancer include endocrine glands cancer, thyroid gland cancer (e.g., thyroid carcinomas/tumors such as papillary, follicular, anaplastic, and medullary), parathyroid gland cancer (e.g., parathyroid carcinoma/tumor), adrenal cortex cancer (e.g., adrenal cortical carcinoma/tumors), pituitary gland cancer (e.g., prolactinoma and craniopharyngioma), thymus cancer, adrenal gland cancer, pineal gland cancer, carotid body cancer, islet cell tumors, paraganglion cancer, pancreatic endocrine tumors (PET) (e.g., non-functional PET, PPoma, gastrinoma, insulinoma, VIPoma, glucagonoma, somatostatinoma, GRFoma, and ACTHoma), and carcinoid tumors.

In some embodiments, cancer mediated by KRAS, for example, by KRAS alterations such as KRAS mutations, or by amplification, overexpression, and/or activation of KRAS wildtype is soft tissue sarcoma. Non-limiting examples of soft tissue sarcoma include fibrosarcoma, fibrous histiocytoma, liposarcoma, leiomyosarcoma, rhabdomyosarcoma, angiosarcoma, lymphangiosarcoma, Kaposi's sarcoma, glomus tumor, hemangiopericytoma, synovial sarcoma, giant cell tumor of tendon sheath, solitary fibrous tumor of pleura and peritoneum, diffuse mesothelioma, malignant peripheral nerve sheath tumor (MPNST), granular cell tumor, clear cell sarcoma, melanocytic schwannoma, plexosarcoma, neuroblastoma, ganglioneuroblastoma, neuroepithelioma, extraskeletal Ewing's sarcoma, paraganglioma, extraskeletal chondrosarcoma, extraskeletal osteosarcoma, mesenchymoma, alveolar soft part sarcoma, epithelioid sarcoma, extrarenal rhabdoid tumor, and desmoplastic small cell tumor.

In some embodiments, cancer mediated by KRAS, for example, by KRAS alterations such as KRAS mutations, or by amplification, overexpression, and/or activation of KRAS wildtype is bone sarcoma. Non-limiting examples of bone sarcoma include myeloma, reticulum cell sarcoma, chondrosarcoma (e.g., central, peripheral, clear cell, and mesenchymal chondrosarcoma), osteosarcoma (e.g., parosteal, periosteal, high-grade surface, small cell, radiation-induced osteosarcoma, and Paget's sarcoma), Ewing's tumor, malignant giant cell tumor, adamantinoma, (fibrous) histiocytoma, fibrosarcoma, chordoma, small round cell sarcoma, hemangioendothelioma, hemangiopericytoma, osteochondroma, osteoid osteoma, osteoblastoma, eosinophilic granuloma, and chondroblastoma.

In some embodiments, cancer mediated by KRAS, for example, by KRAS alterations such as KRAS mutations, or by amplification, overexpression, and/or activation of KRAS wildtype is mesothelioma. Non-limiting examples of mesothelioma include pleural mesothelioma and peritoneal mesothelioma.

In some embodiments, cancer mediated by KRAS, for example, by KRAS alterations such as KRAS mutations, or by amplification, overexpression, and/or activation of KRAS wildtype is skin cancer. Non-limiting examples of skin cancer include basal cell carcinoma, squamous cell carcinoma, Merkel's cell carcinoma, melanoma (e.g., cutaneous, superficial spreading, lentigo maligna, acral lentiginous, nodular, and intraocular melanoma), and actinic keratosis.

In some embodiments, cancer mediated by KRAS, for example, by KRAS alterations such as KRAS mutations, or by amplification, overexpression, and/or activation of KRAS wildtype is neoplasms of the central and peripheral nervous systems. Non-limiting examples of neoplasms of the central and peripheral nervous systems include astrocytoma (e.g., cerebral, cerebellar, diffuse, fibrillary, anaplastic, pilocytic, protoplasmic, and gemistocytary), glioblastoma, gliomas, oligodendrogliomas, oligoastrocytomas, ependymomas, ependymoblastomas, choroid plexus tumors, medulloblastomas, meningiomas, schwannomas (or neurinomas) (e.g., acoustic), hemangioblastomas, hemangiomas, hemangiopericytomas, neuromas, ganglioneuromas, neuroblastomas, retinoblastomas, and spinal axis tumors.

In some embodiments, cancer mediated by KRAS, for example, by KRAS alterations such as KRAS mutations, or by amplification, overexpression, and/or activation of KRAS wildtype is lymphomas or leukemias. Non-limiting examples of lymphomas or leukemias include B-cell non-Hodgkin lymphomas (NHL) (e.g., small lymphocytic lymphoma (SLL), lymphoplasmacytoid lymphoma (LPL), mantle cell lymphoma (MCL), follicular lymphoma (FL), diffuse large cell lymphoma (DLCL), and Burkitt's lymphoma (BL)), T-cell non-Hodgkin lymphomas (e.g., anaplastic large cell lymphoma (ALCL), adult T-cell leukemia/lymphoma (ATLL), cutaneous T-cell lymphoma (CTCL), and peripheral T-cell lymphoma (PTCL)), lymphoblastic T-cell lymphoma (T-LBL), adult T-cell lymphoma, lymphoblastic B-cell lymphoma (B-LBL), cutaneous T-cell lymphoma (CTLC), primary central nervous system lymphoma (PCNSL), immunoblastoma, Hodgkin's disease (HD) (e.g., nodular lymphocyte predominance HD (NLPHD), nodular sclerosis HD (NSHD), mixed-cellularity HD (MCHD), lymphocyte-rich classic HD, and lymphocyte-depleted HD (LDHD)), large granular lymphocyte leukemia (LGL), chronic myelogenous leukemia (CML), acute myelogenous/myeloid leukemia (AML), acute lymphatic/lymphoblastic leukemia (ALL), acute promyelocytic leukemia (APL), chronic B-cell lymphocytic leukemia (or chronic lymphocytic/lymphatic leukemia (CLL)), T-cell prolymphocytic leukemia (PLL), hairy cell leukemia, chronic myelogenous/myeloid leukemia (CML), myeloma, plasmacytoma, multiple myeloma (MM), myelodysplastic syndromes (MDS), juvenile myelomonocytic leukemia (JMML), and chronic myelomonocytic leukemia (CMML).

In some embodiments, cancer mediated by KRAS, for example, by KRAS alterations such as KRAS mutations, or by amplification, overexpression, and/or activation of KRAS wildtype is head and neck cancers. Non-limiting examples of head-and-neck cancers include cancers of the oral cavity, cancers of the throat/pharynx, cancers of the larynx, cancers of the paranasal sinuses and nasal cavity (e.g. sinonasal carcinomas, sinonasal adenocarcinomas), and cancers of the salivary glands.

In some embodiments, cancer mediated by KRAS, for example, by KRAS alterations such as KRAS mutations, or by amplification, overexpression, and/or activation of KRAS wildtype is a cancer selected from pancreatic adenocarcinoma, colorectal adenocarcinoma, lung adenocarcinoma, prostate adenocarcinoma, uterine endometrial carcinoma, cholangiocarcinoma, testicular cancer, cervical squamous cell carcinoma, appendiceal cancer, myelodysplastic syndrome, acute myeloid leukemia (AML), and juvenile myelomonocytic leukemia.

In some embodiments, cancer mediated by KRAS, for example, by KRAS alterations such as KRAS mutations, or by amplification, overexpression, and/or activation of KRAS wildtype is epithelial cancer. Non-limiting examples of epithelial cancer include squamous cell carcinoma (SCC) (e.g., carcinoma in situ, superficially invasive, verrucous carcinoma, pseudosarcoma, anaplastic, transitional cell, and lymphoepithelial), adenocarcinoma (AC) (e.g., well-differentiated, mucinous, papillary, pleomorphic giant cell, ductal, small cell, signet-ring cell, spindle cell, clear cell, oat cell, colloid, adenosquamous, mucoepidermoid, and adenoid cystic), mucinous cystadenocarcinoma, acinar cell carcinoma, large cell carcinoma, small cell carcinoma, neuroendocrine tumors (e.g., small cell carcinoma, paraganglioma, and carcinoid), and oncocytic carcinoma. Epithelial cancers may occur, for example, in the lung, mediastinum, gastrointestinal (GI) tract, reproductive system (including the testis and gynecological system including the ovary, fallopian tube, peritoneum, cervix, vulva, vagina, and uterine body including the endometrium and fundus), breast, endocrine system, soft tissue, bone, mesothelioma, skin, central nervous system and brain, and head and neck.

In some embodiments, cancer mediated by KRAS, for example, by KRAS alterations such as KRAS mutations, or by amplification, overexpression, and/or activation of KRAS wildtype is nonepithelial cancer. Non-limiting examples of nonepithelial cancer include sarcomas (e.g., fibrosarcoma, chondrosarcoma, rhabdomyosarcoma, leiomyosarcoma, hemangiosarcoma, giant cell sarcoma, lymphosarcoma, fibrous histiocytoma, liposarcoma, angiosarcoma, lymphangiosarcoma, and neurofibrosarcoma), lymphoma, melanoma, germ cell tumors, hematological neoplasms, and mixed and undifferentiated carcinomas. Nonepithelial cancers may occur, for example, as sarcomas, lymphomas, or leukemias.

In one embodiment, the disease mediated by alterations in one or more genes in the KRAS/MAPK pathway, for example, by KRAS mutations, or by mutation of other genes in the KRAS/MAPK pathway that leads to activation of KRAS signaling. In some embodiments, the disease or condition is a developmental syndrome or disease driven by the alterations in KRAS/MAPK pathway, for example, by KRAS mutations. The types of KRAS mutations that would be targeted in developmental syndrome or disease include, but are not limited to, KRAS V14I, KRAS Q22R, KRAS P34L, KRAS P34Q, KRAS P34R, KRAS 136M, KRAS T58I, KRAS G60R, KRAS G60S, KRAS G60V, KRAS Y71H, KRAS K147E, KRAS D153V, KRAS V152G. Non-limiting examples of developmental syndrome or disease driven by the alterations in KRAS/MAPK pathway, for example, by KRAS mutations of KRAS wildtype, include Noonan Syndrome (NS), Neurofibromatosis type 1 (NF1), Noonan Syndrome with Multiple Lentigines (NSML) (also referred to as LEOPARD syndrome), Capillary Malformation-Arteriovenous Malformation Syndrome (CM-AVM), Costello Syndrome (CS), Cardio-Facio-Cutaneous Syndrome (CFC), Legius Syndrome (also known as NF1-like Syndrome) and Hereditary gingival fibromatosis.

In some embodiments, the present disclosure relates to administering a compound of Formula (I), or a pharmaceutically acceptable salt thereof, to a subject in need thereof. In some embodiments, administering a compound of Formula (I), or a pharmaceutically acceptable salt thereof, to a subject in need thereof stabilizes a disease mediated by KRAS activity, for example, by KRAS alterations such as KRAS mutations, or by amplification, overexpression, and/or activation of KRAS wildtype. In some embodiments, administering a compound of Formula (I), or a pharmaceutically acceptable salt thereof, to a subject in need thereof enhances the effect of another medication used to treat a disease mediated by KRAS, for example, by KRAS alterations such as KRAS mutations, or by amplification, overexpression and/or activation of KRAS wildtype. In some embodiments, administering a compound of Formula (I), or a pharmaceutically acceptable salt thereof, to a subject in need thereof delays the progression of a disease mediated by KRAS, for example, by KRAS alterations such as KRAS mutations, or by amplification, overexpression, and/or activation of KRAS wildtype. In some embodiments, administering a compound of Formula (I), or a pharmaceutically acceptable salt thereof, to a subject in need thereof increases the quality of life of the subject having a disease mediated by KRAS, for example, by KRAS alterations such as KRAS mutations, or by amplification, overexpression, and/or activation of KRAS wildtype. In some embodiments, administering a compound of Formula (I), or a pharmaceutically acceptable salt thereof, to a subject in need thereof prolongs survival of a subject having a disease mediated by KRAS, for example, by KRAS alterations such as KRAS mutations, or by amplification, overexpression, and/or activation of KRAS wildtype.

In some aspects, provided herein is a method of slowing or delaying progression of a disease mediated by KRAS, for example, by KRAS alterations such as KRAS mutations, or by amplification, overexpression, and/or activation of KRAS wildtype in a subject in need thereof, the method comprising administering a compound of Formula (I), or a pharmaceutically acceptable salt thereof, to the subject. In some embodiments, provided herein is a method of stabilizing a disease mediated by KRAS, for example, by KRAS alterations such as KRAS mutations, or by amplification, overexpression, and/or activation of KRAS wildtype in a subject in need thereof, the method comprising administering a compound of Formula (I), or a pharmaceutically acceptable salt thereof, to the subject. In some embodiments, the method provides a partial or total remission of a disease mediated by KRAS, for example, by KRAS alterations such as KRAS mutations, or by amplification, overexpression, and/or activation of KRAS wildtype in a subject in need thereof, the method comprising administering a compound of Formula (I), or a pharmaceutically acceptable salt thereof, to the subject. In some embodiments, the method increases the quality of life of the subject having a disease mediated by KRAS, for example, by KRAS alterations such as KRAS mutations, or by amplification, overexpression, and/or activation of KRAS wildtype in a subject in need thereof, the method comprising administering a compound of Formula (I), or a pharmaceutically acceptable salt thereof, to the subject. In some embodiments, the method prolongs survival of the subject having a disease mediated by KRAS, for example, by KRAS alterations such as KRAS mutations, or by amplification, overexpression, and/or activation of KRAS wildtype in a subject in need thereof, the method comprising administering a compound of Formula (I), or a pharmaceutically acceptable salt thereof, to the subject.

In another aspect, provided herein is a method of delaying the occurrence or recurrence of a disease mediated by KRAS, for example, by KRAS alterations such as KRAS mutations, or by amplification, overexpression, and/or activation of KRAS wildtype in a subject in need thereof, the method comprising administering a compound of Formula (I), or a pharmaceutically acceptable salt thereof, to the subject.

In further aspects, provided herein is a method of decreasing the dose of one or more other medications required to treat a disease mediated by KRAS, for example, by KRAS alterations such as KRAS mutations, or by amplification, overexpression, and/or activation of KRAS wildtype in a subject in need thereof, the method comprising administering a compound of Formula (I), or a pharmaceutically acceptable salt thereof, to the subject. In some embodiments, provided herein is a method of enhancing the effect of another medication used to treat a disease mediated by KRAS, for example, by KRAS alterations such as KRAS mutations, or by amplification, overexpression, and/or activation of KRAS wildtype in a subject in need thereof, the method comprising administering a compound of Formula (I), or a pharmaceutically acceptable salt thereof, to the subject.

In some aspects, provided herein is a compound of Formula (I), or a pharmaceutically acceptable salt thereof, for use as a drug. In some aspects, provided herein is a compound of Formula (I), or a pharmaceutically acceptable salt thereof, for use in treating a disease mediated by KRAS, for example, by KRAS alterations such as KRAS mutations, or by amplification, overexpression, and/or activation of KRAS wildtype. In some aspects, provided herein is a compound of Formula (I), or a pharmaceutically acceptable salt thereof, for use in the manufacture of a medicament. In other aspects, provided herein is a compound of Formula (I), or a pharmaceutically acceptable salt thereof, for use in the manufacture of a medicament for treating a disease mediated by KRAS, for example, by KRAS alterations such as KRAS mutations, or by amplification, overexpression, and/or activation of KRAS wildtype.

In any of the embodiments above, the disease mediated by KRAS, for example, by KRAS alterations such as KRAS mutations, or by amplification, overexpression, and/or activation of KRAS wildtype, is cancer. In some embodiments, the disease is a developmental disease or syndrome mediated by alterations in one or more genes in the KRAS/MAPK pathway, for example, by KRAS mutations, including, but not limited to, KRAS^{G12A} mutant, KRAS^{G12C} mutant, KRAS^{G12D} mutant, KRAS^{G12R} mutant, KRAS^{G12S} mutant, KRAS^{G12V} mutant, KRAS^{G13A} mutant, KRAS^{G13C} mutant, KRAS^{G13D} mutant, KRAS^{G13R} mutant, KRAS^{G12S} mutant, KRAS^{G13V} mutant, KRAS^{Q61E} mutant, KRAS^{Q61H} mutant, KRAS^{Q61K} mutant, KRAS^{Q61L} mutant, KRAS^{Q61P} mutant, KRAS^{Q61R} mutant, KRAS^{A146P} mutant, KRAS^{A146T} mutant, or KRAS^{A146V} mutant, or by mutation of other genes in the KRAS/MAPK pathway that leads to activation of KRAS signaling. In some embodiments, the disease is a cancer mediated by alterations in one or more genes in the KRAS/MAPK pathway, for example, by KRAS mutations, including, but not limited to, KRAS^{G12A} mutant, KRAS^{G12C} mutant, KRAS^{G12D} mutant, KRAS^{G12R} mutant, KRAS^{G12S} mutant, KRAS^{G12V} mutant, KRAS^{G13A} mutant, KRAS^{G13C}-mutant, KRAS^{G13D} mutant, KRAS^{G13R} mutant, KRAS^{G12S} mutant, KRAS^{G13V} mutant, KRAS^{Q61E} mutant, KRAS^{Q61H} mutant, KRAS^{Q61K} mutant, KRAS^{Q61L} mutant, KRAS^{Q61P} mutant, KRAS^{Q61R} mutant, KRAS^{A146P} mutant, KRAS^{A146T} mutant, or KRAS^{A146V} mutant, or by mutation of other genes in the KRAS/MAPK pathway that leads to activation of KRAS signaling.

In a particular embodiment, the disease is a developmental disease or syndrome or a cancer mediated by alterations in one or more genes in the KRAS/MAPK pathway or and the KRAS mutation is KRAS^{G12A} mutant, KRAS^{G12C} mutant, KRAS^{G12D} mutant, KRAS^{G12S} mutant, KRAS^{G12V} mutant, KRAS^{G13A} mutant, KRAS^{G13D} mutant, KRAS^{G13R} mutant, KRAS^{G12S} mutant, KRAS^{G13V} mutant, KRAS^{Q61E} mutant, KRAS^{Q61H} mutant, KRAS^{Q61L} mutant, KRAS^{Q61P} mutant, KRAS^{Q61R} mutant, KRAS^{A146P} mutant, KRAS^{A146T} mutant, or KRAS^{A146V} mutant, or a combination thereof. In an alternative embodiment, the disease is a developmental disease or syndrome or a cancer mediated by alterations in one or more genes in the KRAS/MAPK pathway or and the KRAS mutation is any activating KRAS mutations or combination thereof, with the exception of KRAS^{G12R}, KRAS^{G13C}, and KRAS^{Q61K}.

In some aspects, provided herein is a method of treating a subject having cancer comprising determining whether a biological sample obtained from the subject is classified as amplified, overexpressed, and/or activated KRAS wild type (KRAS^{WT}) or KRAS mutant and administering to the subject a compound of Formula (I), or a pharmaceutically acceptable salt thereof, if the biological sample is classified as amplified, overexpressed, and/or activated KRAS^{WT}, KRAS^{G12A} mutant, KRAS^{G12C} mutant, KRAS^{G12D} mutant, KRAS^{G12R} mutant, KRAS^{G12S} mutant, KRAS^{G12V} mutant, KRAS^{G13A} mutant, KRAS^{G13C} mutant, KRAS^{G13D} mutant, KRAS^{G13R} mutant, KRAS^{G12S} mutant, KRAS^{G13V} mutant, KRAS^{Q61E} mutant, KRAS^{Q61H} mutant, KRAS^{Q61K} mutant, KRAS mutant, KRAS^{Q61P} mutant, KRAS^{Q61R}, mutant, KRAS^{A146P} mutant, KRAS^{A146T} mutant, or KRAS^{A146V} mutant, among other KRAS mutations and/or KRAS amplification. Methods for detecting a mutation in a KRAS nucleotide sequence are known by those of skill in the art. These methods include, but are not limited to, polymerase chain reaction-restriction fragment length polymorphism (PCR-RFLP) assays, PCR-single strand conformation polymorphism (PCR-SSCP) assays, real-time PCR assays, PCR sequencing, mutant allele-specific PCR amplification (MASA) assays, direct sequencing, primer extension reactions, electrophoresis, oligonucleotide ligation assays, hybridization assays, TaqMan assays, SNP genotyping assays, high resolution melting assays and microarray analyses.

### Modes of Administration

The particular mode of administration and the dosage regimen will be selected by the attending clinician, taking into account the particulars of the case (e.g., the patient, the disease, the disease state involved, and the particular treatment). Treatment may involve daily or multi-daily or less than daily (such as weekly or monthly etc.) doses over a period of a few days to months, or even years. A compound of the disclosure can be administered by any suitable route in the form of a pharmaceutical composition adapted to such a route and in an effective amount (e.g., a dose) for the treatment intended. A compound of the disclosure or a pharmaceutical composition of the disclosure can, e.g., be administered orally, topically, parenterally, or by inhalation, in dosage unit formulations comprising pharmaceutically acceptable excipients. In some embodiments, the compounds of the disclosure are administered orally.

In addition, a compound of the disclosure, a pharmaceutically acceptable salt thereof, or a pharmaceutical composition of the disclosure can be co-administered with other therapeutic agents. The term or expression “co-administration”, “administered in combination with”, and their grammatical equivalents, refer to administration of two or more therapeutic agents to a single patient, and are intended to include treatment regimens in which the agents are administered by the same or different route of administration or at the same or different times. These terms encompass administration of two or more agents to the patient so that both agents and/or their metabolites are present in the patient at the same time. In some embodiments, the compounds of the disclosure and the other agent(s) can be administered in a single composition. In some embodiments, the compounds of the disclosure and the other agent(s) are administered in different compositions.

The compounds of the disclosure, pharmaceutically acceptable salts thereof, or pharmaceutical compositions disclosed herein can be used in combination with one or more additional pharmacologically active substances. For example, the disclosure includes methods of treating a condition/disease/or cancer comprising administering to a subject in need thereof a compound of the disclosure or a pharmaceutically acceptable salt or a pharmaceutical composition disclosed herein thereof in combination with an EGFR (or EGFR mutant) inhibitor, such as afatinib, osimertinib, lapatinib, erlotinib, dacomitinib, poziotinib, neratinib, gefitinib JBJ-04-125-02, alflutinib (AST 2818), almonertinib (HS10296), BBT-176, BI-4020, CH7233163, gilitertinib, JND-3229, lazertinib, nazartinib (EGF 816), PCC-0208027, rezivertinib (BPI-7711), TQB3804, zorifertinib (AZ-3759), or DZD9008; an EGFR antibody such as cetuximab, panitumumab, necitumumab, HLX07, JMT101; or a bispecific EGFR and MET antibody (e.g., amivantamab ((JNJ-61186372, JNJ-372)). Alternatively, a compound of the disclosure, a pharmaceutically acceptable salt thereof or a pharmaceutical composition disclosed herein can be administered in combination with other anti-cancer agents e.g., in combination with MEK, including mutant MEK inhibitors (atebimetinib, trametinib, cobimtetinib, binimetinib, selumetinib, refametinib); c-MET, including mutant c-Met inhibitors (savolitinib, cabozantinib, foretinib, glumetinib, tepotinib) and MET antibodies (emibetuzumab, telisotuzumab vedotin (ABBV 339)); mitotic kinase inhibitors (CDK4/6 inhibitors such as palbociclib, ribociclib, abemacicilb, GIT38); anti-angiogenic agents (e.g., bevacizumab, nintedanib); apoptosis inducers such as Bcl-2 inhibitors e.g, venetoclax, obatoclax, navitoclax, palcitoclax (APG-1252), and Mcl-1 inhibitors e.g., AZD-5991, AMG-176, S-64315; mTOR inhibitors e.g, rapamycin, temsirolimus, everolimus, ridoforolimus; RET inhibitors, like pralsetinib and selpercatinib, and PI3K inhibitors dactolisib (BEZ235), pictilisib (GDC-0941), LY294002, idelalisib (CAL-101); JAK inhibitors (e.g., AZD4205, itacitinib), Aurora A inhibitors (e.g., alisertib); BCR/ABL and/or Src family tyrosine kinase inhibitors (e.g., dasatinib); VEGF inhibitors (e.g., MP0250; ramucirumab); multi-kinase protein inhibitors (e.g., anlotinib, midostaurin); PARP inhibitors (e.g., niraparib); cytotoxic chemotherapy (e.g., platinum therapies (e.g., cisplatin (CDDP), carboplatin (CBDCA), nedaplatin (CDGP), oxaliplatin), 5-fluorouracil, irinotecan, gemcitabine, paclitaxel); PD-1/PD-L1 inhibitors (e.g., durvalumab (MEDI 4736), pembrolizumab, nivolumab, atezolizumab, cemiplimab) including bispecific inhibitors that in part target PD-1/PD-L1 (e.g. SAR445877, ivonescimab, LBL-015); HER2/neu receptor inhibitors (e.g., trastuzumab); anti-HER2 or anti-HER3 antibody-drug conjugates (e.g., patritumab deruxtecan (U3-1402), trastuzumab emtansine); or immunogene therapy (e.g., oncoprex); RAS inhibitors (e.g. sotorasib, adagrasib, daraxonrasib); Anti-estrogen therapy (e.g. tamoxifen, letrozole, anastrozole).

In a particular embodiment, the disclosure includes methods of treating a pancreatic cancer comprising administering to a subject in need thereof a compound of the disclosure or a pharmaceutically acceptable salt or a pharmaceutical composition disclosed herein thereof in combination with FOLFIRINOX (5-fluorouracil, leucovorin, irinotecan and oxaliplatin), modified FOLFIRINOX, gemcitabine plus nab-paclitaxel (Gnp), or modified gemcitabine plus nab-paclitaxel. In a particular embodiment, the disclosure includes methods of treating a colorectal cancer comprising administering to a subject in need thereof a compound of the disclosure or a pharmaceutically acceptable salt or a pharmaceutical composition disclosed herein thereof in combination with FOLFOX (folinic acid (leucovorin), fluorouracil (5-FU), and oxaliplatin (Eloxatin)) and anti-EGFR (cetuximab or panitumumab), FOLFOX and anti-VEGF (bevacizumab), FOLFIRI ((folinic acid (leucovorin), fluorouracil (5-FU), and irinotecan (Camptosar)) and anti-EGFR (cetuximab or panitumumab), FOLRIRI and anti-VEGF (bevacizumab), or anti-EGFR (cetuximab or panitumumab). In a particular embodiment, the disclosure includes methods of treating a NSCLC comprising administering to a subject in need thereof a compound of the disclosure or a pharmaceutically acceptable salt or a pharmaceutical composition disclosed herein thereof in combination with PD-1 (pembrolizumab) with or without chemotherapy (cisplatin/carboplatin with pemetrexed/paclitaxel).

### EXAMPLES

The following Examples are presented by way of illustration and are not intended to limit the scope of the disclosure. One skilled in the art may modify the procedures set forth in the illustrative examples to arrive at the desired products. As appropriate, procedures involving the use of commercially available kits and reagents are generally carried out in accordance with manufacturer-defined protocols and conditions unless otherwise noted.

### ABBREVIATIONS

The following abbreviations may be relevant for this application.

| | | | |
|---|---|---|---|
| AcOH | acetic acid | CDCl₃ | deutero-chloroform |
| aq. | Aqueous | Cs₂CO₃ | cesium carbonate |
| BH₃-Me₂S | borane-dimethyl sulfide | CuBr | copper bromide |
| Boc₂O | di-tert-butyl carbonate | d | doublet |
| (BPin)₂ | bis(pinacolato)diboron | DCM | dichloromethane |
| br | Broad | DePhosPdCl₂ | dichloro[2bis(2- |
| BINAP | 2,2′- | | (diphenylphosphino) |
| | bis(diphenylphosphino)- | | phenyl)ether]palladium(II) |
| | 1,1′-binaphthyl | DHP | 3,4-dihydropyran |
| BINAP Pd G3 | [2′-(amino-κN)[1,1′- | DIAD | diisopropyl |
| | biphenyl]-2-yl-κC][2′- | | azodicarboxylate |
| | (diphenylphosphino)[1,1′- | DIPEA | N,N-diisopropylethylamine |
| | binaphthalen]-2- | DMA | dimethylacetamide |
| | yl]diphenylphosphine- | DMAP | 4-dimethylaminopyridine |
| | κP](methanesulfonato- | DMF | dimethylformamide |
| | κO)-palladium | DMSO | dimethyl sulfoxide |
| t-BuOH | tert-butanol | DMSO-d₆ | hexadeuterodimethyl |
| t-BuOK | potassium tert-butoxide | | sulfoxide |
| tBuBrettPhos | 2-(di-tert- | DPPA | diphenyl phosphoryl azide |
| | butylphosphino)-2′,4′,6′- | DTAD | di-tert-butyl |
| | triisopropyl-3,6- | | azodicarboxylate |
| | dimethoxy-1,1′-biphenyl | Ephos | dicyclohexyl(3-isopropoxy- |
| tBuBrettPhos | [(2-di-tert-butylphosphino- | | 2′,4′,6′-triisopropyl-[1,1′- |
| Pd G3 | 3,6-dimethoxy-2′,4′,6′- | | biphenyl]-2-yl)phosphane |
| | triisopropyl-1,1′-biphenyl)- | LiAlH₄ | lithium aluminum hydride |
| | 2-(2′-amino-1,1′- | LiHMDS | lithium |
| | biphenyl)]palladium(II) | | bis(trimethylsilyl)amide |
| | methanesulfonate | LiOH | lithium hydroxide |
| EPhos Pd G4 | [dicyclohexyl[3-(1- | M | molar |
| | methylethoxy)-2′,4′,6′- | m | multiplet |
| | tris(1-methylethyl)[1,1′- | mg | milligram |
| | biphenyl]-2- | MHz | mega Hertz |
| | yl]phosphine- | MeCN, ACN | acetonitrile |
| | κP](methanesulfonato- | MeI | iodomethane |
| | κO)[2′-(methylamino- | MeMgBr | methyl magnesium bromide |
| | κN)[1,1′-biphenyl]-2-yl- | Me₃O•BF₄ | trimethyloxonium |
| | κC]palladium | | tetrafluoroborate |
| Eq. | Equivalent | MeOH | methanol |
| EtOAc | ethyl acetate | MeOH-d₄ | deutero-methanol |
| EtOH | Ethanol | MgSO₄ | magnesium sulfate |
| | | Ms₂O | methanesulfonic anhydride |
| EtO₂ | ethyl ether | mL | millilitres |
| FA, HCO₂H | formic acid | mmol | millimole |
| g | Gram | MS m/z | mass spectrum peak |
| H | Proton | MTBE | methyl tert-butyl ether |
| HBr | hydrogen bromide | N | normal |
| HCl | hydrochloric acid | N₂ | nitrogen |
| ¹H NMR | proton nuclear magnetic | NaBH₄ | sodium borohydride |
| | resonance | Na₂CO₃ | sodium carbonate |
| HPLC | high-performance liquid | NaH | sodium hydride |
| | chromatography | | |
| IPA/iPrOH | isopropyl alcohol | NaHCO₃ | sodium bicarbonate |
| K₂CO₃ | potassium carbonate | NaI | sodium iodide |
| KOAc | potassium acetate | NaNO₂ | sodium nitrite |
| L | Litre | MS m/z | mass spectrum peak |
| LCMS | liquid chromatography- | PEPPSI-Pd | dichloro[1,3-bis(2,6-Di-3- |
| | mass spectrometry | | pentylphenyl)imidazol-2- |
| LiCl | lithium chloride | | ylidene](3- |
| NaOH | sodium hydroxide | | chloropyridyl)palladium(II) |
| NaOMe | sodium methoxide | PE | petroleum ether |
| NaOt-Bu | sodium tert-butoxide | PPh₃ | triphenylphosphine |
| Na₂SO₄ | sodium sulfate | POCl₃ | phosphorus oxychloride |
| NCS | N-chlorosuccinimide | ppm | parts per million |
| NH₂NH₂•H₂O | hydrazine hydrate | Prep | preparative |
| NH₃ | Ammonia | q | quartet |
| NH₄Cl | ammonium chloride | rt | room temperature |
| NH₄OAc | ammonium acetate | s | singlet |
| NH₄OH | ammonium hydroxide | sat. | saturated |
| Pd(dppf)Cl₂ | [1,1′- | SEMCl | 2-(trimethylsilyl) |
| | bis(diphenylphosphino) | | ethoxymethyl chloride |
| | ferrocene] | SFC | supercritical fluid |
| | dichloropalladium(II) | | chromatography |
| Pd(dtbpf)Cl₂ | [1,1′-bis(di-tert- | t | triplet |
| | butylphosphino) | TBAF | tetra-n-butylammonium |
| | ferrocene] | | fluoride |
| | dichloropalladium(II) | TEA | triethylamine |
| | | TFA | trifluoroacetic acid |
| Pd(PPh₃)Cl₂ | palladium(II) bis | THF | tetrahydrofuran |
| | (triphenylphosphine) | TLC | thin-layer chromatography |
| | dichloride | TMSCl | trimethylsilyl chloride |
| Pd(PPh₃)₄ | tetrakis | TMSCN | trimethylsilyl cyanide |
| | (triphenylphosphine) | TsOH | p-toluenesulfonic acid |
| | palladium(0) | μL | micro litres |
| Pd₂(dba)₃ | tris | μmol | micromole |
| | (dibenzylideneacetone) | | |
| | palladium(0) | | |

### HPLC Conditions

### Acidic

HPLC Method A1: Column: Xselect CSH C18 OBD Column 30*150 mm 5 μm; Mobile Phase A: Water (0.1% FA), Mobile Phase B: MeCN; Flow rate: 30 mL/min;
HPLC Method A2: Column: Xselect CSH C18 OBD Column 30*150 mm 5 μm; Mobile Phase A: Water (0.1% FA), Mobile Phase B: MeCN; Flow rate: 60 mL/min;
HPLC Method A3: Column: Phenomenex luna C18 250*70 mm, 10 μm; mobile phase A: Water (0.225% FA)-MeCN,
HPLC Method A4: Column: Phenomenex luna C18 150*25 mm*10 μm; mobile phase: [Water (0.1% TFA)-MeCN]

### Basic

Method B1: Column: XBridge Prep OBD C18 Column, 30*150 mm, 5 μm; Mobile Phase A: Water (10 mmol/L NH4HCO3)+0.05% NH3·H2O, Mobile Phase B: MeCN; Flow rate: 60 mL/min

### Intermediates

### Intermediate 1

### 2-chloro-4-((S)-1-((S)-1-methylpyrrolidin-2-yl)ethoxy)-7,8-dihydro-5H-pyrano[4,3-d]pyrimidine

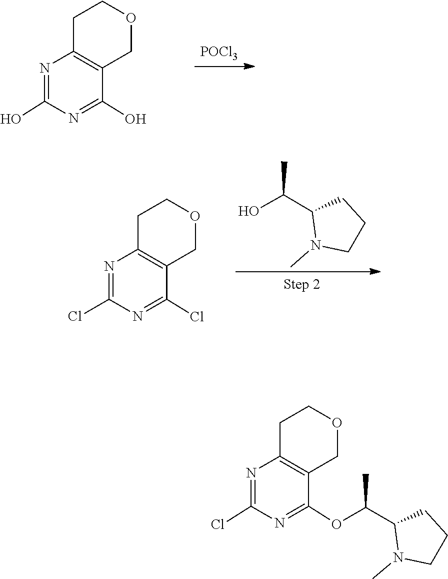

Step 1: Synthesis of 2,4-dichloro-7,8-dihydro-5H-pyrano[4,3-d]pyrimidine

POCl₃ (980 g, 6.39 mol) followed by DIEA (281 g, 2.17 mol) were added to a solution of 7,8-dihydro-5H-pyrano[4,3-d]pyrimidine-2,4-diol (215 g, 1.28 mol) in MeCN (2.15 L) at rt and the mixture stirred at 80° C. for 2 h, then cooled to rt. LiCl (217 g, 5.11 mol) was added and the reaction stirred at 80° C. for 14 h. The cooled mixture was poured into water (3 L), the mixture extracted with DCM (3.5 L×3) and the combined organic extracts were washed with brine (2 L×2), then concentrated in vacuo. The crude product was purified by silica gel chromatography eluting with PE/EtOAc=I/O to 5/1 to give the title compound (89.0 g, 33.2%) as a yellow solid. LCMS m/z=205, 207 [M+H]+

Step 2: Synthesis of 2-chloro-4-((S)-1-((S)-1-methylpyrrolidin-2-yl)ethoxy)-7,8-dihydro-5H-pyrano[4,3-d]pyrimidine

A solution of (1S)-1-((2S)-1-methylpyrrolidin-2-yl)ethanol (12.6 g, 97.5 mmol) and the title compound from step 1 (20.0 g, 97.5 mmol) in THF (200 mL) was degassed and purged with N₂, then cooled to 0° C. An ice-cooled solution of KOtBu (11.0 g, 97.5 mmol) in THF was added and the reaction mixture was stirred at 0° C. for 1 h. The mixture was poured slowly into H₂O (500 mL), extracted with EtOAc (400 L×2) and the combined organic extracts were washed with brine and concentrated in vacuo. The crude product was purified by column chromatography (SiO₂, DCM:MeOH=50/1-5/1) to give the title compound 11.0 g as a yellow solid. LCMS m/z=298, 300 [M+H]+

### Intermediate 2

### 2-chloro-4-((S)-1-((S)-1-methylpyrrolidin-2-yl)ethoxy)-5,7-dihydrofuro[3,4-d]pyrimidine

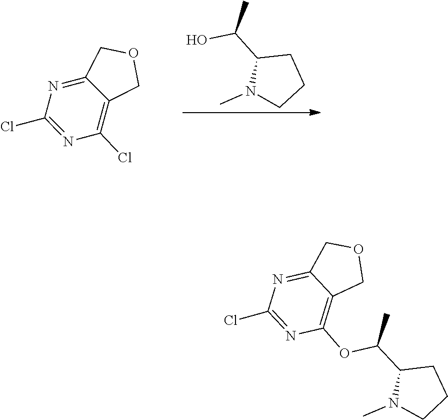

To a solution of 2,4-dichloro-5,7-dihydrofuro[3,4-d]pyrimidine (1.00 g, 5.24 mmol) and (S)-1-((S)-1-methylpyrrolidin-2-yl)ethan-1-ol (676 mg, 5.24 mmol) in dioxane (20 mL) was added NaOtBu (603 mg, 6.28 mmol) and the mixture was stirred at 25° C. for 2 h. The reaction mixture was diluted with water (50 mL) and extracted with EtOAc (30 mL×3). The combined organic layers were washed with brine (30 mL×3), dried over anhydrous Na₂SO₄, filtered and concentrated under reduced pressure. The crude product was purified by reverse-phase HPLC (0.1% formic acid) to give compound the title compound (300 mg, 20%) as yellow oil. LCMS m/z=283 [M+H]+

### Intermediate 3A

### (5R,8S)-2-chloro-4-((S)-1-((S)-1-methylpyrrolidin-2-yl)ethoxy)-6,7,8,9-tetrahydro-5H-5,8-epoxycyclohepta[d]pyrimidine or (5S,8R)-2-chloro-4-((S)-1-((S)-1-methylpyrrolidin-2-yl)ethoxy)-6,7,8,9-tetrahydro-5H-5,8-epoxycyclohepta[d]pyrimidine

### And Intermediate 3B

### (5S,8R)-2-chloro-4-((S)-1-((S)-1-methylpyrrolidin-2-yl)ethoxy)-6,7,8,9-tetrahydro-5H-5,8-epoxycyclohepta[d]pyrimidine or (5R,8S)-2-chloro-4-((S)-1-((S)-1-methylpyrrolidin-2-yl)ethoxy)-6,7,8,9-tetrahydro-5H-5,8-epoxycyclohepta[d]pyrimidine

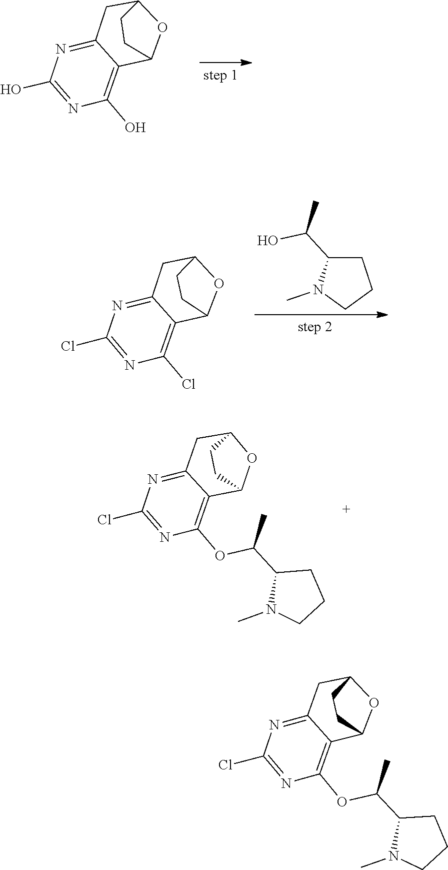

Step 1: Synthesis of 2,4-dichloro-6,7,8,9-tetrahydro-5H-5,8-epoxycyclohepta[d]pyrimidine:

A mixture of 6,7,8,9-tetrahydro-5H-5,8-epoxycyclohepta[d]pyrimidine-2,4-diol (1.6 g, 8.23 mmol) in POCl₃ (20 mL) was stirred at 100° C. for 3 h. The mixture was quenched with ice water, filtered and extracted with DCM (3×100 mL). The organic layer was dried with Na₂SO₄ and concentrated in vacuo. The residue was purified by silica gel column chromatography with PE:EtOAc=4:1 to afford the title compound (1 g, 52.6%) as an off-white oil.

Step 2: Synthesis of (5R,8S)-2-chloro-4-((S)-1-((S)-1-methylpyrrolidin-2-yl)ethoxy)-6,7,8,9-tetrahydro-5H-5,8-epoxycyclohepta[d]pyrimidine and (5S,8R)-2-chloro-4-((S)-1-((S)-1-methylpyrrolidin-2-yl)ethoxy)-6,7,8,9-tetrahydro-5H-5,8-epoxycyclohepta[d]pyrimidine

A mixture of the title compound from step 1 (400 mg, 1.73 mmol), (S)-1-((S)-1-methylpyrrolidin-2-yl)ethan-1-ol (223 mg, 1.73 mmol) and KOtBu (388 mg, 3.46 mmol) in THF (12 mL) was stirred at 80° C. for 3 h. The resulting mixture was filtered and concentrated in vacuo. The mixture was quenched with water, filtered and extracted with DCM (3×80 mL). The organic layer was dried with Na₂SO₄ and concentrated in vacuo. The residue was purified by silica gel column chromatography with DCM:MeOH=20:1. The crude product (130 mg) was purified by Prep-HPLC: (Method B1, Gradient: 34% B to 49% B in 7 min), to afford Peak 1, Intermediate 3A (50 mg, 8.9%) and Peak 2, Intermediate 3B (50 mg, 8.9%) as a yellow solid.

### Intermediate 4A

### (S)-2-chloro-6-methyl-4-((S)-1-((S)-1-methylpyrrolidin-2-yl)ethoxy)-5,6,7,8-tetrahydroquinazolin-6-ol or (R)-2-chloro-6-methyl-4-((S)-1-((S)-1-methylpyrrolidin-2-yl)ethoxy)-5,6,7,8-tetrahydroquinazolin-6-ol

### And Intermediate 4B

### (R)-2-chloro-6-methyl-4-((S)-1-((S)-1-methylpyrrolidin-2-yl)ethoxy)-5,6,7,8-tetrahydroquinazolin-6-ol or (S)-2-chloro-6-methyl-4-((S)-1-((S)-1-methylpyrrolidin-2-yl)ethoxy)-5,6,7,8-tetrahydroquinazolin-6-ol

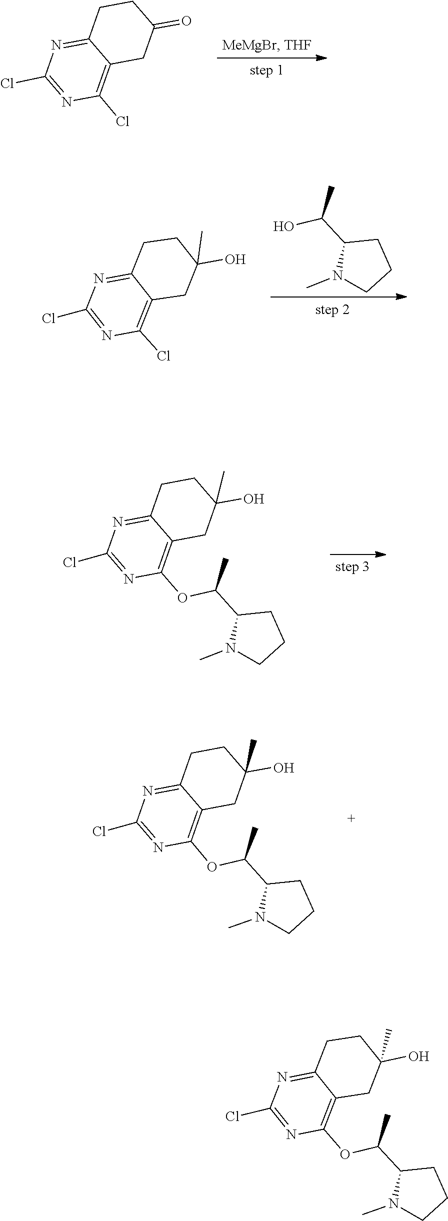

Step 1: Synthesis of 2,4-dichloro-6-methyl-5,6,7,8-tetrahydroquinazolin-6-ol

To a stirred solution of 2,4-dichloro-5,6,7,8-tetrahydroquinazolin-6-one (100 mg, 460 μmol) in Et₂O (3 mL) was added MeMgBr (230 μL, 690 μmol; 3M in Et₂O) dropwise at 0° C. under N₂. The reaction was stirred for 1 h at 0° C. and quenched with aq. citric acid solution. The mixture was extracted with EtOAc (10 mL×3), the combined organic layers were dried over Na₂SO₄ and concentrated. The crude product was purified by prep-TLC with PE:EtOAc=1:1 to give the title compound (25 mg; 23%) as a yellow oil. LCMS m/z=233 [M+H]+

Step 2: Synthesis of 2-chloro-6-methyl-4-((S)-1-((S)-1-methylpyrrolidin-2-yl)ethoxy)-5,6,7,8-tetrahydroquinazolin-6-ol

To a stirred solution of (S)-1-((S)-1-methylpyrrolidin-2-yl)ethan-1-ol (93.0 mg, 720 μmol) in THF (4 mL) was added NaH (28.7 mg, 720 μmol) at 0° C. under N₂ and the mixture stirred for 1 h at rt. The title compound from step 1 (140 mg, 600 μmol) was added at 0° C. and the reaction mixture was stirred for 2 h at rt. The reaction was quenched with ice/water and extracted with EtOAc (10 mL×3). The combined organic layers were dried over Na₂SO₄ and concentrated. The crude product was purified by Prep-TLC with DCM:MeOH=10:1 to give the title compound (70 mg, 35%) as a yellow solid. LCMS m/z=326 [M+H]+

Step 3: Synthesis of (S)-2-chloro-6-methyl-4-((S)-1-((S)-1-methylpyrrolidin-2-yl)ethoxy)-5,6,7,8-tetrahydroquinazolin-6-ol and (R)-2-chloro-6-methyl-4-((S)-1-((S)-1-methylpyrrolidin-2-yl)ethoxy)-5,6,7,8-tetrahydroquinazolin-6-ol

The title compound from step 2 (70 mg, 214 μmol) was purified by prep-HPLC (Method A2, gradient: 1% B to 20% B in 8 min) to give Peak 1, Intermediate 4A (30 mg, 43%) as a white solid and Peak 2, Intermediate 4B (30 mg, 43%) as a white solid. LCMS: m/z=326 [M+H]⁺.

### Intermediate 5A

### (R)-2-chloro-8-methyl-4-((S)-1-((S)-1-methylpyrrolidin-2-yl)ethoxy)-7,8-dihydro-5H-pyrano[4,3-d]pyrimidine or (S)-2-chloro-8-methyl-4-((S)-1-((S)-1-methylpyrrolidin-2-yl)ethoxy)-7,8-dihydro-5H-pyrano[4,3-d]pyrimidine

### And Intermediate 5B

### (S)-2-chloro-8-methyl-4-((S)-1-((S)-1-methylpyrrolidin-2-yl)ethoxy)-7,8-dihydro-5H-pyrano[4,3-d]pyrimidine or (S)-2-chloro-8-methyl-4-((S)-1-((S)-1-methylpyrrolidin-2-yl)ethoxy)-7,8-dihydro-5H-pyrano[4,3-d]pyrimidine

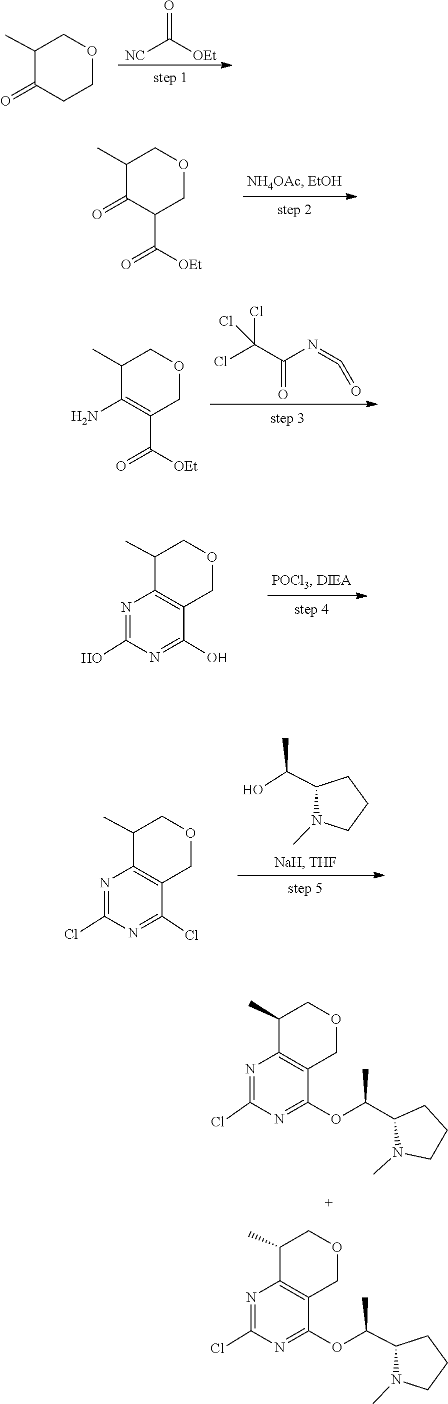

Step 1: Synthesis of ethyl 5-methyl-4-oxotetrahydro-2H-pyran-3-carboxylate

To a stirred solution of 3-methyltetrahydro-4H-pyran-4-one (3.0 g, 26.2 mmol) in THF (30 mL) was added LiHMDS (52.4 mL, 52.4 mmol) at −78° C. and the mixture was stirred for 1 h. Ethyl carbonocyanidate (3.89 g, 39.3 mmol) was added and the reaction was stirred for 2 h at −78° C. Water (30 mL) was added and the mixture was extracted with EtOAc (3×20 mL). The combined organic layers were dried over anhydrous Na₂SO₄ and concentrated. The residue was purified by silica gel column with PE:EtOAc=3:1 to give the title compound (2.0 g, 41%) as a yellow oil. ¹H NMR (400 MHz, CDCl₃) δ 4.08-4.03 (m, 2H), 4.02-3.95 (m, 2H), 3.74-3.57 (m, 2H), 3.18-3.07 (m, 1H), 2.34-2.24 (m, 1H), 1.07-1.05 (m, 3H), 0.99-0.94 (m, 3H).

Step 2: Synthesis of ethyl 4-amino-5-methyl-5,6-dihydro-2H-pyran-3-carboxylate

To a stirred solution of the title compound of step 1 (2.0 g, 10.7 mmol) in EtOH (20 mL) was added NH₄OAc (1.64 g, 21.4 mmol) and the reaction was stirred for 2 h at rt. The reaction mixture was concentrated in vacuo and the residue purified by silica gel chromatography (PE:EtOAc=3:1) to give the title compound (1.5 g, 75%) as a yellow solid. LCMS: m/z=186 [M+H]⁺.

Step 3: Synthesis of 8-methyl-7,8-dihydro-5H-pyrano[4,3-d]pyrimidine-2,4-diol

To a solution of the title compound of step 2 (1.5 g, 8.09 mmol) in MeCN (15 mL) was added 2,2,2-trichloroacetyl isocyanate (3.03 g, 16.1 mmol) at 0° C. and the solution stirred for 0.5 h. NH₃·MeOH (15 mL, 105 mmol) was added and the reaction mixture was stirred for 2 h at rt. The reaction mixture was concentrated in vacuo and the residue was purified by silica gel column (PE:EtOAc=3:1) to give the title compound (1.0 g, 68%) as a white solid. LCMS: m/z=183 [M+H]⁺.

Step 4: Synthesis of 2,4-dichloro-8-methyl-7,8-dihydro-5H-pyrano[4,3-d]pyrimidine

To a stirred solution of the title compound of step 3 (2.0 g, 10.9 mmol) in POCl₃ (20 mL) was added DIEA (2.81 g, 21.8 mmol) and the reaction mixture was stirred for 2 h at 100° C. The cooled mixture was concentrated and adjusted to pH 8 with sat. NaHCO₃ (aq.). The mixture was extracted with EtOAc (3×20 mL) and the combined organic layers were dried over anhydrous Na₂SO₄ and concentrated. The residue was purified by silica gel column with PE: EtOAc=3:1 to give the title compound (1.0 g, 42%) as an off-white solid. LCMS m/z=219 [M+H]+

Step 5: Synthesis of (R)-2-chloro-8-methyl-4-((S)-1-((S)-1-methylpyrrolidin-2-yl)ethoxy)-7,8-dihydro-5H-pyrano[4,3-d]pyrimidine and (S)-2-chloro-8-methyl-4-((S)-1-((S)-1-methylpyrrolidin-2-yl)ethoxy)-7,8-dihydro-5H-pyrano[4,3-d]pyrimidine

To a stirred solution of the title compound of step 4 (1.0 g, 4.56 mmol) and (S)-1-((S)-1-methylpyrrolidin-2-yl)ethan-1-ol (706 mg, 5.47 mmol) in THF (10 mL) was added NaH (364 mg, 9.12 mmol) at 0° C. and the reaction mixture was stirred for 1 h at 0° C. Water (20 mL) was added and the mixture extracted with EtOAc (3×10 mL). The combined organic layers were dried over anhydrous Na₂SO₄ and concentrated. The residue was purified by silica gel column with DCM:MeOH=15:1 to give an oil (500 mg, 35%). This was further purified by Prep-SFC (Column: (R, R)-WHELK-O1-Kromasil, 3*25 cm, 5 μm; Mobile Phase A: CO₂, Mobile Phase B: MeOH; Flow rate: 100 mL/min; Gradient: isocratic 15% B), to give Peak 1, Intermediate 5A (110 mg, 21%) as a yellow oil and Peak 2, Intermediate 5B (115 mg, 21%) as a yellow oil.

Intermediate 5A: LCMS: m/z=312 [M+H]+

Intermediate 5B: LCMS: m/z=312 [M+H]+

### Intermediate 6A

### (S)-2-chloro-6-(methoxymethyl)-4-((S)-1-((S)-1-methylpyrrolidin-2-yl)ethoxy)-6,7-dihydro-5H-cyclopenta[d]pyrimidine or (R)-2-chloro-6-(methoxymethyl)-4-((S)-1-((S)-1-methylpyrrolidin-2-yl)ethoxy)-6,7-dihydro-5H-cyclopenta[d]pyrimidine

### And Intermediate 6B

### (R)-2-chloro-6-(methoxymethyl)-4-((S)-1-((S)-1-methylpyrrolidin-2-yl)ethoxy)-6,7-dihydro-5H-cyclopenta[d]pyrimidine or (S)-2-chloro-6-(methoxymethyl)-4-((S)-1-((S)-1-methylpyrrolidin-2-yl)ethoxy)-6,7-dihydro-5H-cyclopenta[d]pyrimidine

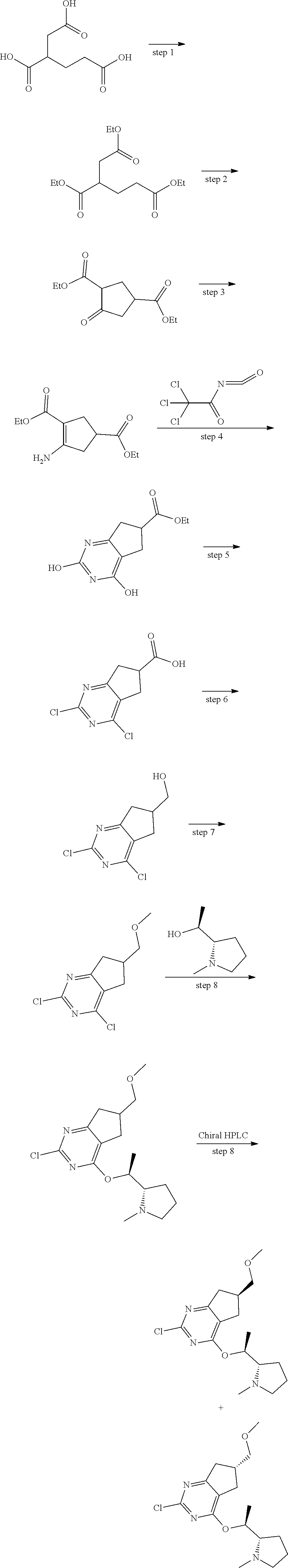

Step 1: Synthesis of triethyl butane-1,2,4-tricarboxylate

To a stirred solution of butane-1,2,4-tricarboxylic acid (35.0 g, 184 mmol) in EtOH (350 mL) was added conc. H₂SO₄ (0.1 mL) and the reaction was stirred for 24 h at 80° C. The mixture was concentrated in vacuo, the residue diluted with water and extracted with DCM (3×300 mL). The combined organic layers were dried over Na₂SO₄ and evaporated under reduced pressure to give the title compound (50.0 g, crude) as a yellow oil. ¹H NMR (400 MHz, CDCl₃) δ 4.20-4.09 (m, 6H), 2.95-2.81 (m, 1H), 2.78-2.68 (m, 1H), 2.53-2.32 (m, 3H), 2.06-1.83 (m, 2H), 1.32-1.20 (m, 9H).

Step 2: Synthesis of diethyl 4-oxocyclopentane-1,3-dicarboxylate

To a stirred solution of the title compound from step 1 (50.0 g, 182 mmol) in toluene (300 mL) was added EtONa (61.5 g, 182 mmol; 20% in EtOH) and the reaction mixture was stirred for 4 h at 100° C. The mixture was cooled to rt and adjusted to pH 7 with 2 M HCl. The resulting solution was extracted with DCM (3×300 mL), the combined organic layers were dried over Na₂SO₄ and concentrated. The crude product was purified by silica gel column with PE:EtOAc=3:1 to give the title compound (13.5 g, 33%) as a yellow oil. ¹H NMR (400 MHz, CDCl₃) δ 4.29-4.09 (m, 4H), 3.68-3.49 (m, 1H), 3.49-3.01 (m, 1H), 2.93-2.15 (m, 3H), 2.14-1.78 (m, 1H), 1.32-1.23 (m, 6H).

Step 3: Synthesis of diethyl 4-aminocyclopent-3-ene-1,3-dicarboxylate

The title compound was obtained from the title compound from step 2 and NH₄OAc, 3.2 g, 24%, as a yellow oil, following a similar procedure to that described in Intermediate 5A/B, step 2. LCMS: m/z=228 [M+H]+

Step 4: Synthesis of ethyl 2,4-dihydroxy-6,7-dihydro-5H-cyclopenta[d]pyrimidine-6-carboxylate

To a solution of the title compound from step 3 (3.2 g, 14.0 mmol) in MeCN (30 mL) was added 2,2,2-trichloroacetyl isocyanate (5.27 g, 28.0 mmol) at 0° C. and the mixture was stirred for 45 mins at rt. The reaction mixture was evaporated under reduced pressure and the residue was dissolved in EtOH (30 mL). EtONa (10.9 g, 32.1 mmol; 20% in EtOH) was added and the reaction was stirred for 16 h at rt. The reaction mixture was adjusted to pH 5 with 6 M HCl and evaporated under reduced pressure to give the title compound (2.9 g, crude) as a yellow oil. LCMS: m/z=225 [M+H]⁺.

Step 5: Synthesis of 2,4-dichloro-6,7-dihydro-5H-cyclopenta[d]pyrimidine-6-carboxylic acid

The title compound was obtained as a brown solid, 2 g, 67%, from the title compound from step 4, following a similar procedure to that described in Intermediate 5A and 5B, step 4. LCMS: m/z=233 [M+H]⁺.

Step 6: Synthesis of (2,4-dichloro-6,7-dihydro-5H-cyclopenta[d]pyrimidin-6-yl)methanol

To a stirred solution of the title compound from step 5 (1.7 g, 7.29 mmol) in THE (20.0 mL) was added BH₃ (14.5 mL, 14.5 mmol; 1 M in THF) and the reaction was stirred for 1 h at rt. Water was added at 0° C. and the resulting solution was extracted with DCM (3×40 mL). The combined organic layers were dried over Na₂SO₄ and concentrated in vacuo. The crude product was purified by silica gel column with PE:EtOAc=1:1 to give the title compound (1.0 g, 63%) as a yellow oil. LCMS: m/z=219 [M+H]⁺.

Step 7: Synthesis of 2,4-dichloro-6-(methoxymethyl)-6,7-dihydro-5H-cyclopenta[d]pyrimidine

To a stirred solution of the title compound from step 6 (1.0 g, 4.56 mmol) in DCM (10.0 mL) were added Me₃O·BF₄ (1.34 g, 9.12 mmol) and 1,8-bis(dimethylamino)naphthalene (2.91 g, 13.6 mmol) and the reaction mixture was stirred for 1.5 h at rt. Water was added, and the mixture was extracted with DCM (3×30 mL). The combined organic layers were dried over Na₂SO₄ and concentrated. The crude product was purified by silica gel column with PE:EtOAc=2:1 to give the title compound (880 mg, 83%) as a yellow oil. LCMS: m/z=233 [M+H]⁺.

Step 8: Synthesis of 2-chloro-6-(methoxymethyl)-4-((S)-1-((S)-1-methylpyrrolidin-2-yl)ethoxy)-6,7-dihydro-5H-cyclopenta[d]pyrimidine

To a stirred solution of the title compound from step 7 (830 mg, 3.56 mmol) in toluene (10 mL) were added NaOtBu (683 mg, 7.12 mmol) and (S)-1-((S)-1-methylpyrrolidin-2-yl)ethan-1-ol (551 mg, 4.27 mmol) and the reaction mixture was stirred for 2 h at 80° C. The cooled mixture was concentrated and purified by Prep-TLC with DCM:MeOH=7:1 to give the title compound (720 mg, 63%) as a yellow oil. LCMS: m/z=326 [M+H]⁺.

Step 9: Synthesis of (S)-2-chloro-6-(methoxymethyl)-4-((S)-1-((S)-1-methylpyrrolidin-2-yl)ethoxy)-6,7-dihydro-5H-cyclopenta[d]pyrimidine and (R)-2-chloro-6-(methoxymethyl)-4-((S)-1-((S)-1-methylpyrrolidin-2-yl)ethoxy)-6,7-dihydro-5H-cyclopenta[d]pyrimidine

The title compound from step 8 (720 mg, 2.20 mmol) was purified by Prep-Chiral-HPLC (Column: CHIRALPAK IC, 2*25 cm, 5 μm; Mobile Phase A: Hex (0.2% IPA), Mobile Phase B: Hex: IPA=9:1; Flow rate: 20 mL/min; Gradient: 15% B isocratic in 20 min) to give Peak 1, Intermediate 6A (220 mg, 31%) as a light yellow oil and Peak 2, Intermediate 6B (250 mg, 35%) as a light yellow oil.

Peak 1, Intermediate 6A: LCMS: m/z=326 [M+H]+

Peak 2, Intermediate 6B: LCMS m/z=326 [M+H]+

### Intermediate 7

### tert-butyl (4-bromo-3-cyano-5,7-difluorobenzo[b]thiophen-2-yl)carbamate

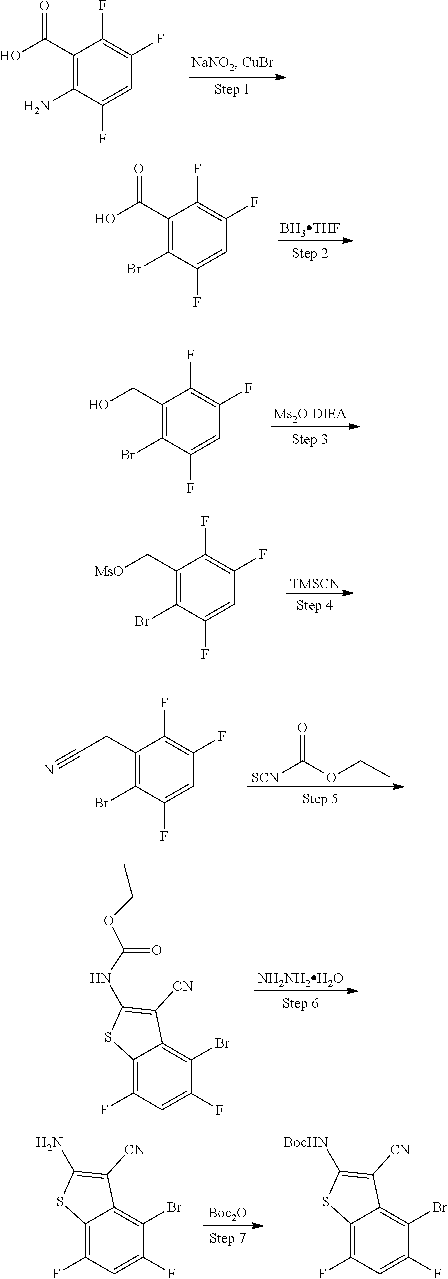

Step 1: Synthesis of 2-bromo-3,5,6-trifluorobenzoic acid

Three reactions were conducted in parallel. To an ice-cooled solution of 2-amino-3,5,6-trifluorobenzoic acid (150 g, 776 mmol) in MeCN (750 mL) was added HBr (750 mL). A mixture of NaNO₂ (58.9 g, 853 mmol) in H₂O (75.0 mL) was added and the reaction mixture was stirred at 0° C. for 1 h. CuBr (133 g, 931 mmol) was added and the mixture was stirred at 70° C. for 1 h. The three reaction mixtures were cooled, combined and concentrated in vacuo. The residue was dissolved in EtOAc, washed with brine, dried over Na₂SO₄, filtered and evaporated under reduced pressure to give the title compound, as a crude yellow liquid, 550 g, ¹H NMR: (400 MHz, DMSO-d₆) δ 7.94-7.86 (m, 1H).

Step 2: Synthesis of (2-bromo-3,5,6-trifluorophenyl)methanol

To a solution of the title compound of step 1 (150 g, 588 mmol) in THF (1.50 L) was added BH₃·THF (1.0 M, 1.29 L) dropwise at 0° C. under N₂. The mixture was slowly heated to 60° C. and stirred for 12 h under N₂, then cooled to 0° C. Sat. aq. K₂CO₃ (1.0 L) was added and the layers separated. The aqueous phase was extracted with DCM (1.0 L×3), the combined organic phase was washed with brine (500 mL), dried with Na₂SO₄, filtered and concentrated in vacuo at 50° C. The residue was purified by column chromatography (SiO₂, PE/EtOAc=10/1 to 3/1), to give the title compound as a yellow solid, 63 g. ¹H NMR: (400 MHz, CDCl₃) δ 7.07-7.01 (m, 1H), 4.88 (s, 2H), 2.25 (br s, 1H).

Step 3: Synthesis of 2-bromo-3,5,6-trifluorobenzyl methanesulfonate

To a solution of the title compound of step 2 (214 g, 864 mmol) in THF (2.14 L) was added DIEA (190 g, 1.47 mol) and Ms₂O (210 g, 1.21 mol) and the reaction mixture was stirred at 20° C. for 12 h. The mixture was filtered, the filtrate was diluted with water (1.0 L), the layers separated and the aqueous phase was extracted with EtOAc (500 mL×3). The combined organic extracts were washed with brine (500 mL), dried with Na₂SO₄, filtered and concentrated in vacuo at 50° C. to give the title compound as a yellow oil, 275 g, crude. ¹H NMR (400 MHz, CDCl₃) δ 7.19-7.13 (m, 1H), 5.42 (s, 2H), 3.10 (s, 3H).

Step 4: Synthesis of 2-(2-bromo-3,5,6-trifluorophenyl) acetonitrile

Three reactions were conducted in parallel containing 137 g, 137 g and 81 g of the title compound from step 3, respectively. The three reactions were performed employing the molar equivalents and, in the manner, described as follows. To a solution of the title compound of step 3 (137 g, 430 mmol) in MeCN (2.06 L) was added TMSCN (85.5 g, 861 mmol) and K₂CO₃ (119 g, 861 mmol) and the mixture degassed and purged with N₂. The reaction mixture was stirred at 80° C. for 2 h. The reaction was quenched by addition to sat. NaHCO₃ solution (2.0 L) at 0° C. under N₂ and extracted with EtOAc (1.0 L×3). The combined organic layers were washed with brine (1.0 L), dried over Na₂SO₄, filtered and concentrated in vacuo. The crude residue of the three parallel reactions were combined and purified by column chromatography (SiO₂, PE/EtOAc=10/1 to 5/1) to give the title compound, 242 g, as a yellow solid. ¹H NMR (400 MHz, CDCl₃) δ 7.15-7.09 (m, 1H), 3.93 (s, 2H).

Step 5: Synthesis of ethyl (4-bromo-3-cyano-5,7-difluorobenzo[b]thiophen-2-yl)carbamate

To a solution of the title compound from step 4 (60 g, 231 mmol) in DMF (600 mL) was added KOtBu (27.2 g, 242 mmol) at 0° C. and the solution stirred at 0° C. for 30 mins. O-Ethyl carbonisothiocyanatidate (31.8 g, 242 mmol) was added and the reaction mixture was stirred at 20° C. for 1 h, then 100° C. for 1 h. The cooled mixture was filtered and the filtrate evaporated under reduced pressure to give the title compound (89.0 g, crude) as a yellow solid. LCMS m/z=307 [M+H]+

Step 6: Synthesis of 2-amino-4-bromo-5,7-difluorobenzo[b]thiophene-3-carbonitrile

To a solution of the title compound from step 5 (89.0 g) in EtOH (445 mL) was added NH₂NH₂·H₂O (84.3 ml) under N₂ and the reaction mixture was stirred at 80° C. for 2 h. The mixture was cooled to rt, then concentrated in vacuo. The crude product was purified by prep-HPLC (Column: I.D.100 mm*H 400 mm Phenomenex Luna C18 (2) 15 μm; 100A; Flow rate 350 mL/min; Mobile phase: H₂O+MeCN; Gradient B %: 50% to 10% over 30 min) to give the title compound as a yellow solid, 29.4 g, 40% over two steps. LCMS m/z=289 [M+H]+

Step 7: Synthesis of tert-butyl (4-bromo-3-cyano-5,7-difluorobenzo[b]thiophen-2-yl)carbamate

To a solution of the title compound from step 6 (29.4 g, 99.5 mmol) in THF (50.0 mL) was added Boc₂O (26.0 g, 119 mmol), DMAP (1.22 g, 9.96 mmol) and DIEA (25.7 g, 199 mmol) and the reaction mixture was stirred at 20° C. for 16 h. The reaction mixture was concentrated in vacuo and the residue triturated with MeCN (50 mL) twice, the solid filtered off and dried to afford the title compound as a yellow solid, 31.6 g, 79.7%. LCMS m/z=388.9 [M+H]+

### Intermediate 8

### tert-butyl (4-bromo-5-chloro-3-cyano-7-fluorobenzo[b]thiophen-2-yl)carbamate

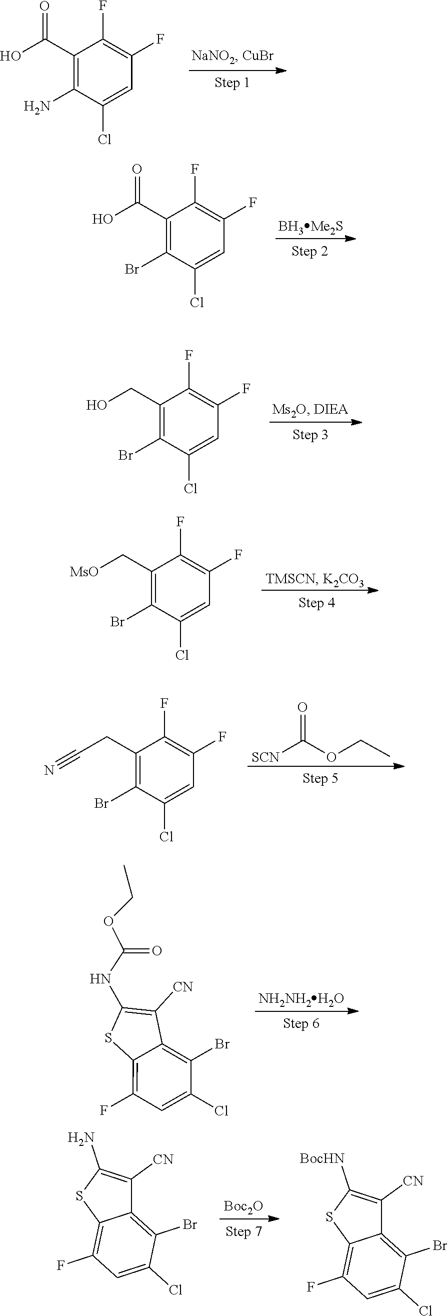

Step 1: Synthesis of 2-bromo-3-chloro-5,6-difluorobenzoic acid

To a solution of 2-amino-3-chloro-5,6-difluorobenzoic acid (65.0 g, 313 mmol) in MeCN (800 mL) and HBr (800 mL) was added CuBr (51.7 g, 360 mmol) and NaNO₂ (23.8 g, 345 mmol) in H₂O (80 mL) at 0° C. and the reaction mixture was stirred at 70° C. for 2 h. Two reactions were combined for work up. The mixture was concentrated in vacuo, the solution was poured into brine (1 L), the aqueous layer was separated and extracted with EtOAc (2×1 L). The combined organic layers were dried over anhydrous Na₂SO₄, filtered and concentrated in vacuo to give the title compound (104.5 g, 58.5%) as a yellow solid. ¹H NMR (400 MHz, CDCl₃) δ 8.00 (br t, 1H)

Step 2: Synthesis of (2-bromo-3-chloro-5,6-difluorophenyl)methanol

To a solution of the title compound of step 1 (52.0 g, 191 mmol) in THF (500 mL) was added BH₃-Me₂S (10 M, 95.8 mL) at 0° C. under N₂ and the reaction mixture was stirred at 20° C. for 16 h. Two reactions were combined for work up. The reaction mixture was quenched with MeOH (100 mL) at 0° C., then concentrated in vacuo. The residue was purified by silica gel chromatography (ISCO®; SepaFlash® Column, Eluent of 0~10% EtOAc/PE gradient @ 100 mL/min) to give the title compound (77.0 g, 55.4%) as a yellow solid. ¹H NMR: (400 MHz, CDCl₃) δ 7.26 (dd, 1H), 4.81 (d, 2H), 3.65 (br s, 1H)

Step 3: Synthesis of 2-bromo-3-chloro-5,6-difluorobenzyl methanesulfonate

The title compound was obtained, as a yellow solid, 92 g, crude, from the title compound from step 2, following a similar procedure to that described in Intermediate 7, step 3. 1H NMR: (400 MHz, DMSO-d₆) δ 8.19-8.08 (m, 1H), 5.45-5.40 (m, 2H), 3.33 (s, 3H)

Step 4: Synthesis of 2-(2-bromo-3-chloro-5,6-difluorophenyl) acetonitrile

The title compound was obtained as a yellow solid, 52 g, 50%, from the title compound of step 3, following a similar prcedure to that described in Intermediate 7, step 4. 1H NMR: (400 MHz, CDCl₃) δ 7.42-7.33 (m, 1H), 3.88 (d, 2H)

Step 5: Synthesis of ethyl (4-bromo-5-chloro-3-cyano-7-fluorobenzo[b]thiophen-2-yl)carbamate

The title compound was obtained as a yellow solid, from the title compound of step 4, following a similar procedure to that described in Intermediate 7, step 5. LCMS m/z=377 [M−H]−

Step 6: Synthesis of 2-amino-4-bromo-5-chloro-7-fluorobenzo[b]thiophene-3-carbonitrile

To a solution of the title compound from step 5 (28.0 g, 74.2 mmol) in EtOH (140 mL) was added NH₂NH₂·H₂O (71.3 g, 1.42 mol) and the reaction mixture was stirred at 80° C. for 6 h. The mixture was cooled to 20° C., filtered and the resulting solid dried to afford the title compound (19.0 g, crude) as a yellow solid. LCMS m/z=305 [M−H]−

Step 7: Synthesis of tert-butyl (4-bromo-5-chloro-3-cyano-7-fluorobenzo[b]thiophen-2-yl)carbamate

To a solution of the title compound from step 6 (9.0 g, 29.5 mmol) in THF (90 mL) was added Boc₂O (19.3 g, 88.4 mmol), TEA (4.47 g, 44.2 mmol) and DMAP (1.80 g, 14.7 mmol) and the reaction mixture was stirred at 25° C. for 2 h. The mixture was filtered, the collected solid triturated with MeOH and filtered to give the title compound as a white solid, (5.71 g, 46.1%). LCMS m/z=405 [M−H]−

### Intermediate 9

### tert-butyl (3-cyano-4-(5,5-dimethyl-1,3,2-dioxaborinan-2-yl)-7-fluorobenzo[b]thiophen-2-yl)carbamate

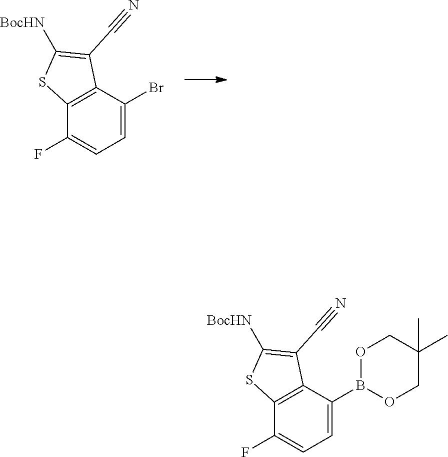 [SMILES: CC(C)(C)OC(=O)Nc1sc2c(F)ccc(Br)c2c1C#N ; CC1(C)COB(c2ccc(F)c3sc(NC(=O)OC(C)(C)C)c(C#N)c23)OC1]

KOAc (79.3 g, 808 mmol) and 5,5,5′,5′-tetramethyl-2,2′-bi(1,3,2-dioxaborinane) (231 g, 1.02 mol) were added to tert-butyl (4-bromo-3-cyano-7-fluorobenzo[b]thiophen-2-yl)carbamate (100 g, 269 mmol) in dioxane (1.80 L) and the mixture was stirred at 50° C. for 1 h. DePhosPdCl₂ (19.2 g, 26.9 mmol) was added and the reaction mixture stirred at 95° C. for 1 h. 5 reactions were combined for work-up. The cooled mixture was filtered and the filter cake washed with PE/MTBE (1:1, 35 L). The filtrate was concentrated in vacuo, the residue suspended in DCM (3.5 L), filtered through silica gel, rinsing through with EtOAc:heptane (1:1, 35 L). The filtrate was washed with saturated NH₄Cl solution, then saturated brine and concentrated in vacuo. The crude product was purified by silica gel chromatography (PE/EtOAc=5/1) and the product was triturated with heptane: 1,2-dimethoxyethane=5/1 at rt for 30 min. The mixture was filtered, the filter cake washed with PE and dried in vacuo to give the title compound, 560 g, 49%, as a yellow solid. LCMS m/z=281 [M+H]+

### Intermediate 10

### tert-butyl (3-cyano-4-(5,5-dimethyl-1,3,2-dioxaborinan-2-yl)-7-fluorobenzo[b]thiophen-2-yl)((2-(trimethylsilyl)ethoxy)methyl)carbamate

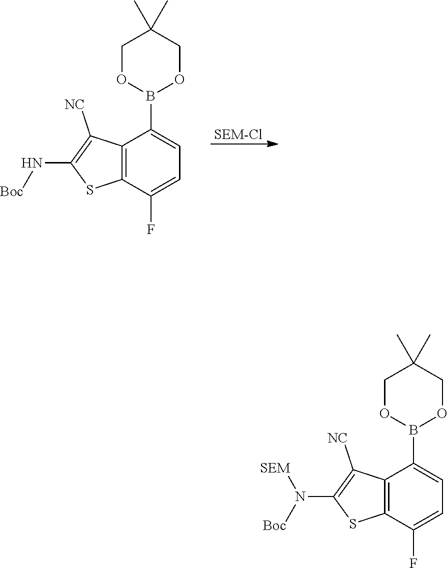 [SMILES: CC1(C)COB(c2ccc(F)c3sc(NC(=O)OC(C)(C)C)c(C#N)c23)OC1 ; "SEM-Cl" ; CC1(C)COB(c2ccc(F)c3sc(N(COCC[Si](C)(C)C)C(=O)OC(C)(C)C)c(C#N)c23)OC1]

K₂CO₃ (273 g, 1.98 mol) and SEMCl (495 g, 2.97 mol) were added to a solution of Intermediate 9 (400 g, 989 mmol) in DMF (2.0 L) and the reaction mixture stirred at rt for 12 h. The mixture was poured into water (6.0 L) and extracted with EtOAc (2.0 L×2). The combined organic extracts were washed with brine (2.0 L), dried (Na₂SO₄), filtered and concentrated in vacuo. The residue was triturated with PE (1.0 L) at rt for 1 h, filtered and dried under vacuum to give the title compound, 255 g, 46.5%, as a white solid. ¹H NMR (400 MHz, DMSO-d₆) δ 7.72-7.75 (m, 1H), 7.37-7.42 (m, 1H), 5.14 (s, 2H), 3.74 (s, 4H), 3.63-3.67 (m, 2H), 1.46 (s, 9H), 1.03 (s, 6H), 0.93-0.97 (s, 2H), 0.01 (s, 9H).

### Intermediate 11

### tert-butyl (3-cyano-4-(5,6-dichloro-3-fluoro-1H-indazol-4-yl)-7-fluorobenzo[b]thiophen-2-yl)carbamate

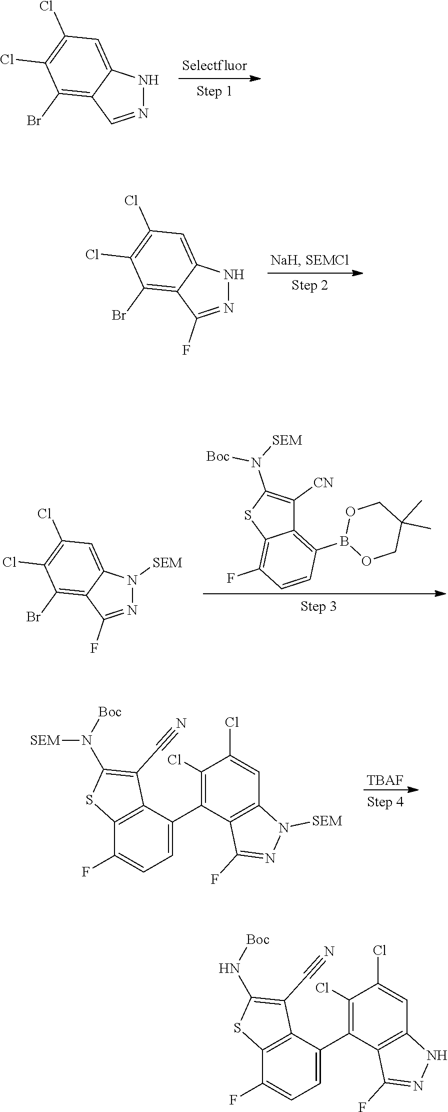

Step 1: Synthesis of 4-bromo-5,6-dichloro-3-fluoro-1H-indazole

Ten reactions were performed in parallel as follows. 4-Bromo-5,6-dichloro-1H-indazole 1 (500 mg, 1.88 mmol) and SelectFluor (1.33 g, 3.76 mmol) were taken up into a microwave tube in MeCN (10 mL) under N₂. The sealed tube was heated at 100° C. for 2 hours under microwave. The cooled reaction mixtures were combined and was concentrated in vacuo and purified by silica gel column chromatography (PE:EtOAc=1:0 to 5:1) to give the title compound (3.40 g, 64%) as a yellow solid. ¹H NMR (400 MHz, DMSO-d₆): δ ppm 13.18 (s, 1H), 7.91 (s, 1H).

Step 2: Synthesis of 4-bromo-5,6-dichloro-3-fluoro-1-((2-(trimethylsilyl)ethoxy)methyl)-1H-indazole

To a solution of the title compound from step 1 (3.00 g, 10.6 mmol) in THF (75 mL) was added NaH (507 mg, 12.7 mmol, 60% in mineral oil) at 0° C. under N₂. SEMCl (2.11 g, 12.7 mmol) was added and the reaction mixture stirred at rt for 25 mins. The reaction mixture was quenched with aq. NH₄Cl solution (100 mL) and extracted with EtOAc (80 mL×3). The combined organic layers were dried over Na₂SO₄, filtered and concentrated under reduced pressure. The residue was purified by silica gel column chromatography (PE:EtOAc=1:0 to 10:1) to give the title compound (3.50 g, 80%) as a yellow solid. ¹H NMR (400 MHz, DMSO-d₆) δ ppm 8.34 (s, 1H), 5.66 (s, 2H), 3.54-3.52 (m, 2H), 0.82-0.79 (m, 2H), 0.09 (s, 9H).

Step 3: Synthesis of tert-butyl (3-cyano-4-(5,6-dichloro-3-fluoro-1-((2-(trimethylsilyl)ethoxy)methyl)-1H-indazol-4-yl)-7-fluorobenzo[b]thiophen-2-yl)((2-(trimethylsilyl)ethoxy)methyl)carbamate

A mixture of Intermediate 10 (4.13 g, 7.73 mmol), the title compound from step 2 (3.20 g, 7.73 mmol), DePhosCl₂ (1.66 g, 2.32 mmol) and Cs₂CO₃ (7.55 g, 23.2 mmol) in dioxane (40 mL) was degassed and purged with N₂ and the reaction mixture was stirred at 100° C. for 16 h under N₂. The reaction mixture was concentrated under reduced pressure and the residue was purified by silica gel column chromatography (PE:EtOAc=1:0 to 10:1) to give the title compound (1.20 g, 21%) as a yellow solid. ¹H NMR (400 MHz, CDCl₃) δ ppm 7.80 (d, 1H), 7.40-7.36 (m, 1H), 7.32-7.28 (m, 1H), 5.65-5.49 (m, 2H), 5.14-5.06 (m, 2H), 3.70-3.67 (m, 2H), 3.61-3.56 (m, 2H), 1.43 (s, 9H), 1.03-1.00 (m, 2H), 0.95-0.91 (m, 2H), 0.02 (s, 9H), 0.02 (s, 9H).

Step 4: Synthesis of tert-butyl (3-cyano-4-(5,6-dichloro-3-fluoro-1H-indazol-4-yl)-7-fluorobenzo[b]thiophen-2-yl)carbamate

To a solution of the title compound from step 3 (1.20 g, 1.59 mmol) in THF (15 mL) was added TBAF (1 M in THF, 15 mL) and the reaction mixture was stirred at 60° C. for 16 h. The reaction mixture was diluted with water (50 mL) and extracted with EtOAc (50 mL×3). The combined organic layers were dried over Na₂SO₄, filtered and concentrated in vacuo. The residue was purified by silica gel column chromatography (PE:EtOAc=1:0 to 3:1) to give the title compound (460 mg, 39%) as a yellow solid. LCMS m/z=495 [M+H]+

### Intermediate 12

### tert-butyl (3-cyano-7-fluoro-4-(3-fluoro-5-methyl-1H-indazol-4-yl)benzo[b]thiophen-2-yl)carbamate

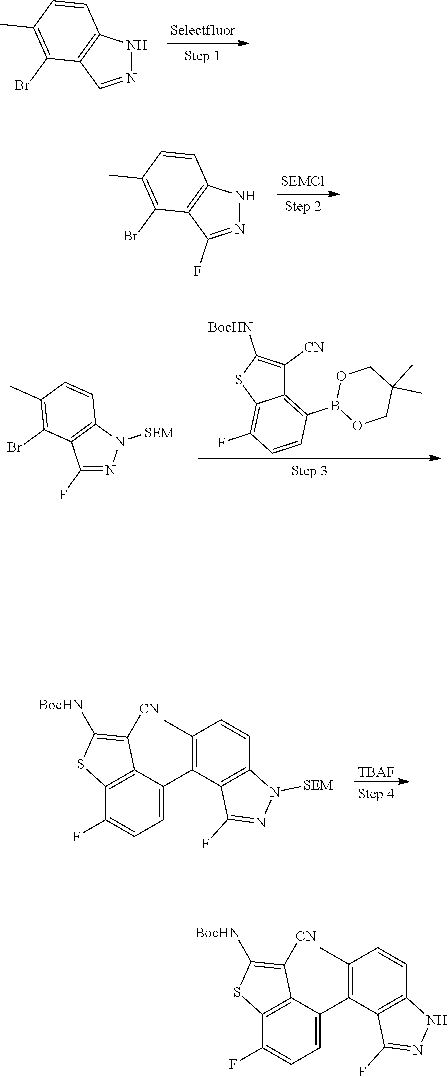

Step 1: Synthesis of 4-bromo-3-fluoro-5-methyl-1H-indazole

A solution of 4-bromo-5-methyl-1H-indazole (135 g, 639 mmol) and DMA (3.0 L) was degassed and purged with N₂. Selectfluor (453 g, 1.28 mol) was added at rt and the reaction mixture stirred at 70° C. for 2 h. The cooled mixture was poured into water, the mixture extracted with EtOAc, the combined organic layers washed with brine, dried over Na₂SO₄ and concentrated in vacuo. The residue was purified by column chromatography (SiO₂, PE/EtOAc=100/1 to 20/1). The product was further purified by prep-HPLC (Method A3, gradient: 38%-68% B over 30 min). The product containing fractions were concentrated in vacuo and the aqueous phase was extracted with EtOAc (150 mL×3). The combined organic layers were washed with brine (150 mL), dried over Na₂SO₄ and evaporated under reduced pressure to give the title compound (14.5 g, 9.9%) as a white solid. LCMS m/z=231 [M+H]+

Step 2: Synthesis of 4-bromo-3-fluoro-5-methyl-1-((2-(trimethylsilyl)ethoxy)methyl)-1H-indazole

A mixture of the title compound from step 1 (38.0 g, 165 mmol), Cs₂CO₃ (108 g, 331 mmol) and DMF (380 mL) was degassed and purged with N₂ then cooled to 0° C. SEMCl (96.8 g, 580 mmol) was added, and the reaction mixture was stirred at 25° C. for 2 h. The mixture was poured into water (500 mL), then extracted with EtOAc (150 mL×3). The combined organic layers were washed with brine (150 mL), dried over Na₂SO₄ and evaporated under reduced pressure to give the title compound (59.0 g, crude) as a yellow oil. LCMS m/z=361 [M+H]+

Step 3: Synthesis of tert-butyl (3-cyano-7-fluoro-4-(3-fluoro-5-methyl-1-((2-(trimethylsilyl)ethoxy)methyl)-1H-indazol-4-yl)benzo[b]thiophen-2-yl)carbamate

Pd(dppf)Cl₂ (24.0 g, 32.8 mmol) and Cs₂CO₃ (107 g, 328 mmol) were added to a solution of the title compound from step 2 (59.0 g, 164 mmol), Intermediate 9 (66.4 g, 164 mmol) and dioxane (1.0 L) and the mixture was degassed and purged with N₂. The reaction mixture was stirred at 90° C. for 2 h. The cooled mixture was poured into water (1.0 L) and then extracted with EtOAc (1.0 L×2). The combined organic layers were washed with brine (1.0 L), dried over Na₂SO₄ and concentrated. The residue was purified by column chromatography (SiO₂, PE/EtOAc=100/1 to 2/1) to give the title compound (56 g, 59.8%) as a white solid. LCMS m/z=593 [M+H]+

Step 4: Synthesis of tert-butyl (3-cyano-7-fluoro-4-(3-fluoro-5-methyl-1H-indazol-4-yl)benzo[b]thiophen-2-yl)carbamate

A solution of the title compound from step 3 (56.0 g, 98.1 mmol) in TBAF (1.0 M, 560 mL) was pumped at 8 mL/min to a flow reactor (SS, Coils reactor, 3.175 (⅛″) mm, 120 mL, 150° C.) with a pressure of 4 Mpa and the reaction mixture collected in a bottle over 85 min. The mixture was cooled to 20° C., poured into water (500 mL) and extracted with EtOAc (250 mL×3). The combined organic layers were washed with brine (200 mL), dried over Na₂SO₄, concentrated in vacuo and the residue was purified by column chromatography (SiO₂, PE/(DCM:THF=5:1)=10/1 to 3/1). The product was triturated with MeCN to give the title compound as a yellow solid, 22.9 g, 51.3%. LCMS m/z=441 [M+H]+.

### Intermediate 13

### tert-butyl (4-(3-chloro-5-methyl-1H-indazol-4-yl)-3-cyano-7-fluorobenzo[b]thiophen-2-yl)carbamate

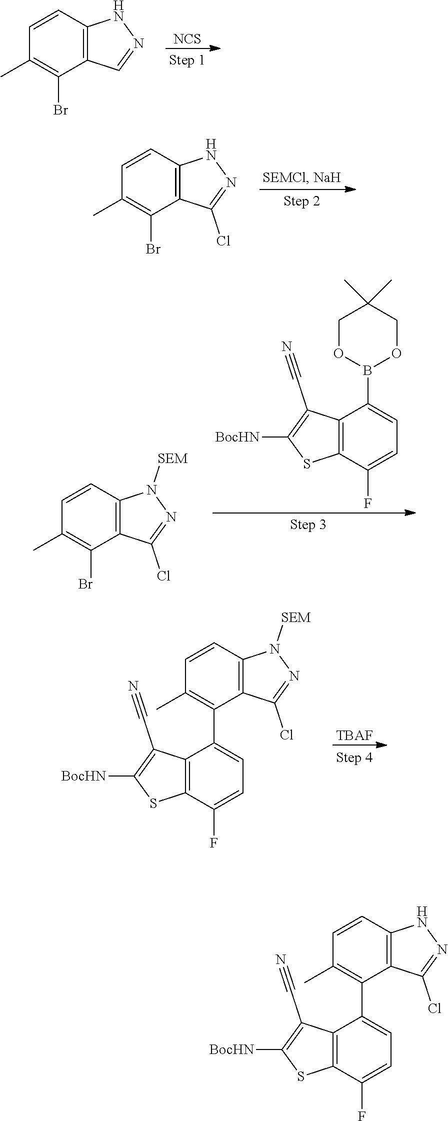

Step 1: Synthesis of 4-bromo-3-chloro-5-methyl-1H-indazole

To a solution of 4-bromo-5-methyl-1H-indazole (50.0 g, 237 mmol) in MeCN (500 mL) was added NCS (34.8 g, 260 mmol) and the reaction mixture was stirred at 65° C. for 6 h. The solution was poured into sat aq. NaHCO₃ (1 L), the aqueous layer was separated and further extracted with EtOAc (2×1 L). The combined organic layers were dried over anhydrous Na₂SO₄, filtered and evaporated under reduced pressure to give the title compound as a white solid, (65 g, crude). LCMS: m/z=247 [M+H]⁺

Step 2: Synthesis of 4-bromo-3-chloro-5-methyl-1-((2-(trimethylsilyl)ethoxy)methyl)-1H-indazole

The title compound was obtained as a yellow oil, 100 g, 95%, from the title compound from step 1 and SEMCl, following a similar procedure to that described in Intermediate 11, step 2. LCMS: m/z=319 [M+H]⁺

Step 3: Synthesis of tert-butyl (4-(3-chloro-5-methyl-1-((2-(trimethylsilyl)ethoxy)methyl)-1H-indazol-4-yl)-3-cyano-7-fluorobenzo[b]thiophen-2-yl)carbamate

To a solution of the title compound from step 2 (32 g, 85.2 mmol) in dioxane (300 mL) was added K₂CO₃ (23.5 g, 170.3 mmol), Intermediate 9 (41.3 g, 102 mmol) and Pd-118 (5.6 g, 8.5 mmol) and the reaction mixture was stirred at 100° C. for 16 h. The solution was poured into brine (2 L), the aqueous layer was separated and further extracted with EtOAc (2×1 L). The combined organic layers were dried over anhydrous Na₂SO₄, filtered and concentrated in vacuo. The residue was purified by silica gel chromatography (ISCO®; SepaFlash® Silica Column, Eluent of 0~20% EtOAc/PE gradient @ 60 mL/min) to give the title compound (7 g, 7.0%) as a yellow solid.

Step 4: Synthesis of tert-butyl (4-(3-chloro-5-methyl-1H-indazol-4-yl)-3-cyano-7-fluorobenzo[b]thiophen-2-yl)carbamate

A solution of the title compound of step 3 (14 g, 23.8 mmol) in TBAF (1 M, 140 mL) was stirred at 145° C. for 16 h. The solution was poured into H₂O (100 mL), the aqueous layer was separated and further extracted with EtOAc (2×500 mL). The combined organic layers were dried over anhydrous Na₂SO₄, filtered and concentrated in vacuo. The crude product was triturated with EtOAc at 20° C. for 30 min, the mixture was filtered and the solid dried to give the title compound as a white solid, (2 g, 18.4%). LCMS: m/z=457 [M+H]+

### Intermediate 14

### tert-butyl (4-(5-chloro-3-fluoro-1H-indazol-4-yl)-3-cyano-7-fluorobenzo[b]thiophen-2-yl)carbamate

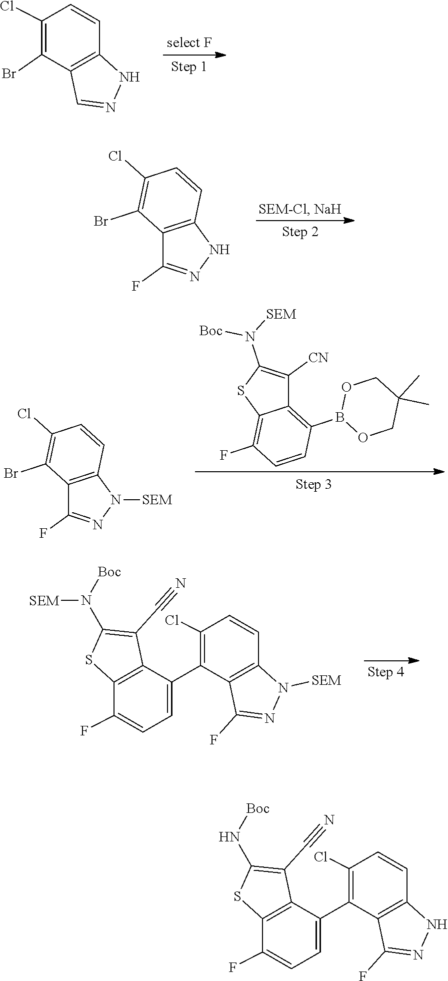

Step 1: Synthesis of 4-bromo-5-chloro-3-fluoro-1H-indazole

SelectFluor (1.99 g, 5.62 mmol) was added to a solution of 4-bromo-5-chloro-1H-indazole (1.00 g, 4.32 mmol, two batches) in MeCN (10 mL) and the reaction mixture was stirred at 100° C. for 2 h under microwave irradiation. The reaction mixture was concentrated under reduced pressure and the residue was purified by prep-HPLC [column: Phenomenex luna C18 150*40 mm*15 μm; mobile phase: [water (formic acid)-MeCN]; gradient: 42%-72% B over 15 min) to afford the title compound (650 mg, 30%) as an off-white solid. LCMS m/z=251 [M+H]+

Step 2: Synthesis of 4-bromo-5-chloro-3-fluoro-1-((2-(trimethylsilyl)ethoxy)methyl)-1H-indazole

The title compound was obtained as a colorless oil, 600 mg, 71%, from the title compound of step 1, following a similar procedure to that described in Intermediate 13, step 2. 1H NMR (400 MHz, CDCl₃) δ ppm 7.49-7.46 (m, 1H), 7.42-7.38 (m, 1H), 5.55 (s, 2H), 3.59-3.53 (m, 2H), 0.92-0.84 (m, 2H), −0.01-−0.06 (m, 9H).

Step 3: Synthesis of tert-butyl (4-(5-chloro-3-fluoro-1-((2-(trimethylsilyl)ethoxy)methyl)-1H-indazol-4-yl)-3-cyano-7-fluorobenzo[b]thiophen-2-yl) ((2-(trimethylsilyl)ethoxy)methyl)carbamate

A mixture of the title compound of step 2 (450 mg, 1.19 mmol), Intermediate 10 (950 mg, 1.78 mmol), DePhosPdCl₂ (254 mg, 356 μmol) and Cs₂CO₃ (1.16 g, 3.56 mmol) in dioxane (9 mL) was degassed and purged with N₂, then stirred at 100° C. for 4 h. The cooled reaction mixture was filtered, washed with EtOAc (20 mL), then diluted with water (20 mL) and extracted with EtOAc (20 mL×2). The combined organic layers were washed with brine (30 mL×2), dried over anhydrous Na₂SO₄, filtered and concentrated in vacuo. The residue was purified by column chromatography (SiO₂, PE/EtOAc=100/1 to 1/1) to afford the title compound (600 mg, 70%) as yellow solid. LCMS m/z=720 [M+H]⁺

Step 4: Synthesis of tert-butyl (4-(5-chloro-3-fluoro-1H-indazol-4-yl)-3-cyano-7-fluorobenzo[b]thiophen-2-yl)carbamate

The title compound was obtained as a yellow solid, 250 mg, 65%, from the title compound from step 3, following a similar procedure to that described in Intermediate 11, step 4. LCMS m/z=460 [M+H]+

### Intermediate 15

### tert-butyl (4-(5-chloro-3-methyl-1H-indazol-4-yl)-3-cyano-7-fluorobenzo[b]thiophen-2-yl)carbamate

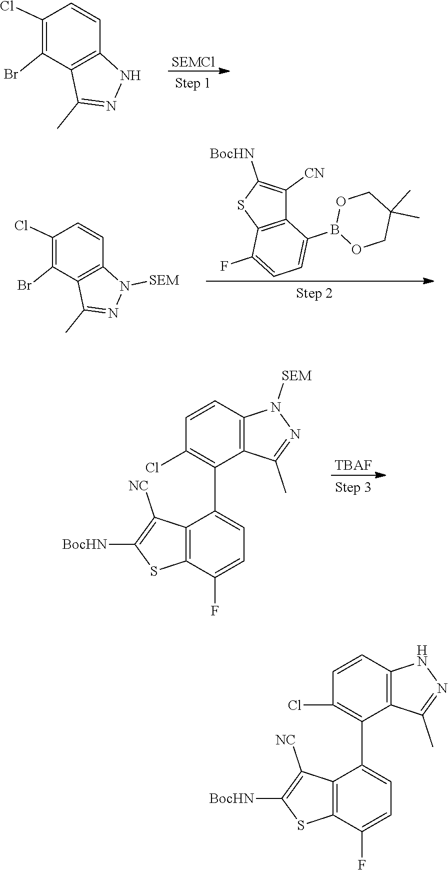

Step 1: Synthesis of 4-bromo-5-chloro-3-methyl-1-((2-(trimethylsilyl)ethoxy)methyl)-1H-indazole

The title compound was obtained as a yellow oil, 235 g, 63.8%, from 4-bromo-5-chloro-3-methyl-1H-indazole and SEMCl, following a similar procedure to that described in Intermediate 13, step 2. LCMS m/z=377 [M+H]+.

Step 2: Synthesis of tert-butyl (4-(5-chloro-3-methyl-1-((2-(trimethylsilyl)ethoxy)methyl)-1H-indazol-4-yl)-3-cyano-7-fluorobenzo[b]thiophen-2-yl)carbamate

K₂CO₃ (87.9 g, 636 mmol), Intermediate 9 (140 g, 190 mmol) and Pd (dtbpf) C12 (10.3 g, 15.9 mmol) were added to a solution of the title compound of step 1 (72.0 g, 159 mmol) in dioxane (720 mL) and the reaction mixture was stirred at 80° C. for 3 h under N₂. 2 reactions were combined for workup. The cooled mixture was poured into water (3 L) and the mixture extracted with EtOAc (1.5 L×2). The combined organic extracts were washed with brine (1.2 L), dried over Na₂SO₄ and concentrated in vacuo. The crude product was triturated with DCM at 20-25° C. for 10 min, the mixture filtered, washing through with DCM (200 mL). The filtrate was concentrated in vacuo and the product purified by reverse-phase HPLC (0.10% FA condition) to give the title compound, 24.0 g, 11.8% as a brown solid. LCMS m/z=588 [M+H]⁺

Step 3: Synthesis of tert-butyl (4-(5-chloro-3-methyl-1H-indazol-4-yl)-3-cyano-7-fluorobenzo[b]thiophen-2-yl)carbamate

A solution of the title compound of step 2 (23.0 g, 39.1 mmol) in TBAF (240 mL) was pumped at 4 mL/min to flow reactor 1 (SS,Coils reactor, 3.175 (⅛″) mm, 60.0 mL, 150° C.). The residence time of flow reactor 1 was 15 min. The mixture was collected in a bottle for 100 mins. The mixture was poured into H₂O (3.0 L) and stirred at rt for 10 min. The mixture was extracted with EtOAc (1.50 L×3), the combined organic phase was washed with brine (2.0 L), dried over Na₂SO₄ and concentrated in vacuo. The crude product was purified by HPLC (Method A3, gradient: 20%-50% B over 30 min). The aqueous residue was extracted with EtOAc (1.80 L×2), the combined organic extracts dried over Na₂SO₄ and evaporated under reduced pressure to give the title compound (13.5 g, 74.6%) as an off-white solid. LCMS m/z=457 [M+H]+

### Intermediate 16

### tert-butyl (4-(5-chloro-1H-indazol-4-yl)-3-cyano-7-fluorobenzo[b]thiophen-2-yl)carbamate

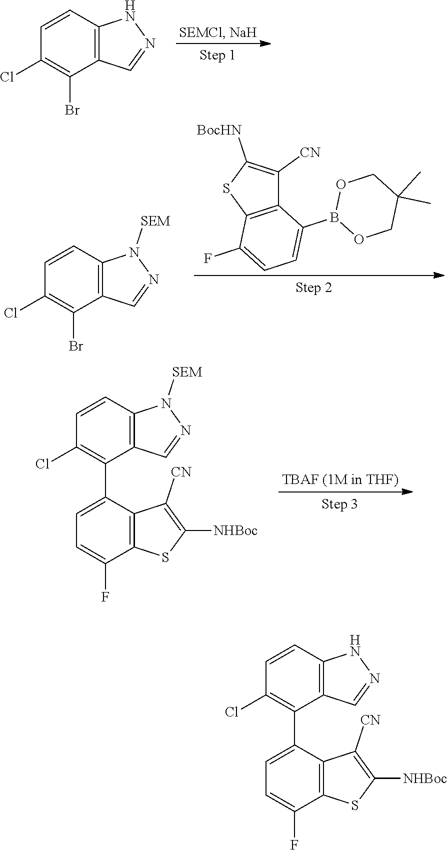

Step 1: Synthesis of 4-bromo-5-chloro-1-((2-(trimethylsilyl)ethoxy)methyl)-1H-indazole

The title compound was obtained as a yellow oil, 140 g, 94.3%, from 4-bromo-5-chloro-1H-indazole and SEMCl, following a similar procedure to that described in Intermediate 13, step 2. LCMS: m/z=362.9 (M+H)⁺

Step 2: Synthesis of tert-butyl (4-(5-chloro-1-((2-(trimethylsilyl)ethoxy)methyl)-1H-indazol-4-yl)-3-cyano-7-fluorobenzo[b]thiophen-2-yl)carbamate

The title compound was obtained as a brown solid, 101 g, 28.3%, from the title compound of step 1 and Intermediate 9, following the procedure described in Intermediate 15, step 2. LCMS m/z=573 [M+H]+

Step 3: Synthesis of tert-butyl (4-(5-chloro-1H-indazol-4-yl)-3-cyano-7-fluorobenzo[b]thiophen-2-yl)carbamate

The title compound was obtained as an off-white solid, 23.1 g, 36.4%, from the title compound of step 2, following a similar procedure to that described in Intermediate 15, step 3. LCMS m/z=443 [M+H]+

### Intermediate 17

### tert-butyl (3-cyano-4-(3,5-dichloro-1H-indazol-4-yl)-7-fluorobenzo[b]thiophen-2-yl)carbamate

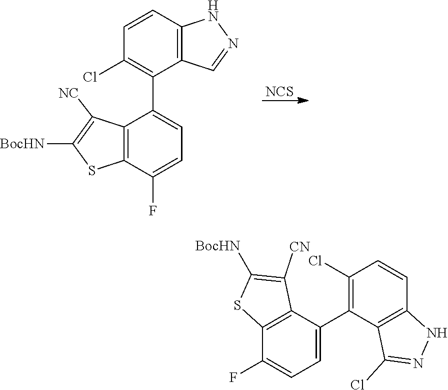 [SMILES: CC(C)(C)OC(=O)Nc1sc2c(F)ccc(-c3c(Cl)ccc4[nH]ncc34)c2c1C#N ; "NCS" ; CC(C)(C)OC(=O)Nc1sc2c(F)ccc(-c3c(Cl)ccc4[nH]nc(Cl)c34)c2c1C#N]

NCS (29.6 g, 222 mmol) was added to a solution of Intermediate 16 (100 g, 201 mmol) in MeCN (1 L) and the reaction mixture stirred at 60° C. for 6 h. Three reaction batches were combined. The reaction mixture was poured into 10% aq. Na₂SO₃ (2.50 L) and extracted with 2-MeTHF (3.0 L×2). The combined organic extracts were washed with brine (2.0 L), dried over Na₂SO₄ filtered, and concentrated in vacuo. The crude product was purified by reverse-phase HPLC (0.1% FA). The product containing fractions were concentrated in vacuo, the pH adjusted to 8 using saturated Na₂CO₃ solution and the aqueous solution extracted with 2-MeTHF (2.0 L×2). The combined organic extracts were washed with brine (1.50 L), dried over Na₂SO₄, filtered, and concentrated in vacuo. The crude product (75.0 g) was triturated with MTBE (300 mL) for 3 h, the mixture filtered and the solid dried to give the title compound, 59.5 g, as a yellow solid. ¹H NMR (400 MHz, DMSO-d₆) δ 13.57 (s, 1H), 11.58 (s, 1H), 7.68 (d, 1H), 7.57 (d, 1H), 7.36 (d, 2H), 1.50 (s, 9H)

### Intermediate 18

### tert-butyl (3-cyano-7-fluoro-4-(1H-indazol-4-yl)benzo[b]thiophen-2-yl)carbamate

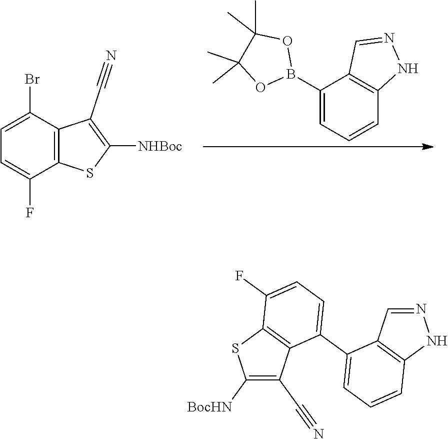 [SMILES: CC(C)(C)OC(=O)Nc1sc2c(F)ccc(Br)c2c1C#N ; CC1(C)OB(c2cccc3[nH]ncc23)OC1(C)C ; CC(C)(C)OC(=O)Nc1sc2c(F)ccc(-c3cccc4[nH]ncc34)c2c1C#N]

K₂CO₃ (156 g, 1.13 mol) and 4-(4,4,5,5-tetramethyl-1,3,2-dioxaborolan-2-yl)-1H-indazole (207 g, 849 mmol) were added to a solution of (tert-butyl (4-bromo-3-cyano-7-fluorobenzo[b]thiophen-2-yl)carbamate (210 g, 566 mmol) in dioxane (2.10 L) and H₂O (210 mL) at 20° C. and the mixture purged with N₂. Pd(dppf)Cl₂ (41.4 g, 56.6 mmol) was added, the mixed purged with N₂ and the reaction mixture heated at 105° C. for 16 h. The cooled reaction was quenched with water, the mixture filtered and the filtered solid dried under vacuum. The solid was triturated with heptane, filtered and dried to give the title compound, 160 g, 67.7%. LCMS m/z=409 [M+H]⁺

### Intermediate 19

### tert-butyl (4-bromo-3-cyano-5,7-difluorobenzo[b]selenophen-2-yl)carbamate

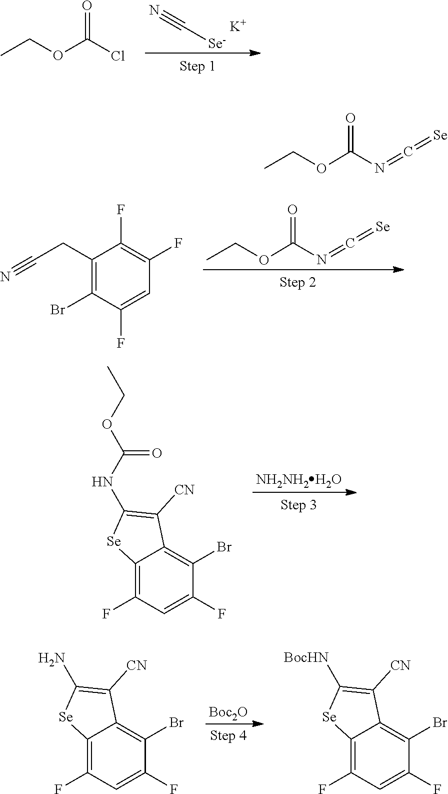

Step 1: Synthesis of O-ethyl carbonisoselenocyanatidate

To a solution of potassium selenocyanate (266 g, 1.85 mol) in toluene (2.0 L) was added H₂O (3.33 g, 184 mmol) and pyridine (1.46 g, 18.4 mmol) at 20° C. under N₂. Ethyl carbonochloridate (200 g, 1.85 mol) was added dropwise and the reaction mixture was stirred at 30° C. for 3 h under N₂. The reaction mixture was filtered, and the filter cake was washed with toluene (500 mL×3). The filtrate was concentrated under reduced pressure to give the title compound (57.0 g, crude) as a brown solid.

Step 2: Synthesis of ethyl (4-bromo-3-cyano-5,7-difluorobenzo[b]selenophen-2-yl)carbamate

The title compound was obtained from the title compounds of step 1 and Intermediate 7, step 4, 65.5 g, 58.5%, following a similar procedure to that described in Intermediate 7, step 5. LCMS m/z 409 [M+H]+

Step 3: Synthesis of 2-amino-4-bromo-5,7-difluorobenzo[b]selenophene-3-carbonitrile

The title compound was obtained as a yellow solid, 18.1 g, 43%, from the title compound of step 2 and NH₂NH₂·H₂O, following a similar procedure to that described in Intermediate 7, step 6. LCMS m/z=337 [M+H]+

Step 4: Synthesis of tert-butyl (4-bromo-3-cyano-5,7-difluorobenzo[b]selenophen-2-yl)carbamate

The title compound was obtained as an off-white solid, from the title compound of step 3, 20.8 g, 87%, following a similar procedure to that described in Intermediate 7, step 7. LCMS m/z=437 [M+H]⁺

### Intermediate 20

### tert-butyl (4-bromo-3-cyano-7-fluorobenzo[b]selenophen-2-yl)carbamate

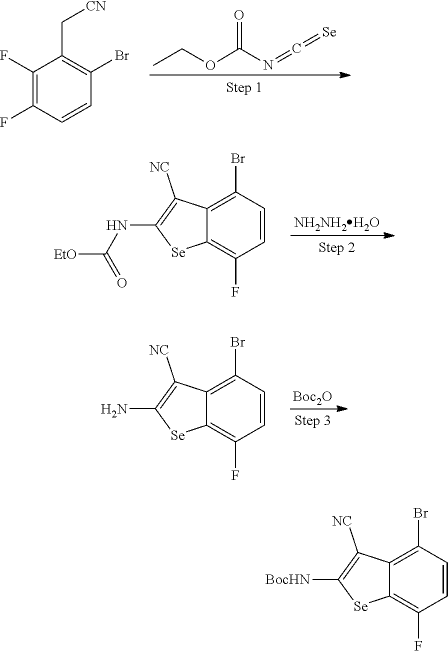

Step 1: Synthesis of ethyl (4-bromo-3-cyano-7-fluorobenzo[b]selenophen-2-yl)carbamate

To a solution of 2-(6-bromo-2,3-difluorophenyl) acetonitrile (28.1 g, 120.9 mmol) in DMF (280 mL) was added KOtBu (14.2 g, 127 mmol) at 0° C. and the mixture was stirred at this temperature for 30 mins. Intermediate 19, step 1 (28.0 g, crude) in DMF (280 mL) was added dropwise at 0° C. and the reaction mixture was stirred at 25° C. for 1 h and then 100° C. for 30 min. The reaction was poured into ice water (1.5 L) and filtered. The crude product was triturated with EtOAc at 25° C. for 30 min to give the title compound (13 g, 27.5%) as a gray solid. LCMS m/z=389 [M−H]⁻.

Step 2: Synthesis of 2-amino-4-bromo-7-fluorobenzo[b]selenophene-3-carbonitrile

Two reactions were conducted in parallel containing, 26 g and 20.5 g of the title compound from step 1, respectively. The two reactions were performed employing the molar equivalents and, in the manner, described as follows. To a solution of the title compound of step 1 (26.0 g, 66.6 mmol) in EtOH (130 mL) was added NH₂NH₂·H₂O (24.2 g, 410 mmol, 85% purity) and the reaction mixture was stirred at 80° C. for 3 h. The reaction mixture was concentrated in vacuo. The residues of the two parallel reactions were combined and triturated with DCM (250 mL) to give the title compound, 35.3 g, as a gray solid. LCMS m/z=319 [M−H

Step 3: Synthesis of tert-butyl (4-bromo-3-cyano-7-fluorobenzo[b]selenophen-2-yl)carbamate

To a solution of the title compound of step 2 (20.0 g, 62.9 mmol) in DMF (200 mL) was added DMAP (768 mg, 6.29 mmol), DIEA (16.3 g, 126 mmol) and Boc₂O (16.5 g, 75.5 mmol) and the reaction mixture was stirred at 25° C. for 16 h. The reaction mixture was concentrated in vacuo and the residue was purified by prep-HPLC (column: Welch Ultimate XB-CN 250×70×10 μm; mobile phase: [Hexane-EtOH]; gradient: 1%-33% B over 19 mins). The product was triturated with iPr₂O to give the title compound as a white solid, (9.98 g, 21.5%). LCMS m/z=417 [M−H]−

### Intermediate 21

### tert-butyl (4-bromo-3-cyanobenzo[b]selenophen-2-yl)carbamate

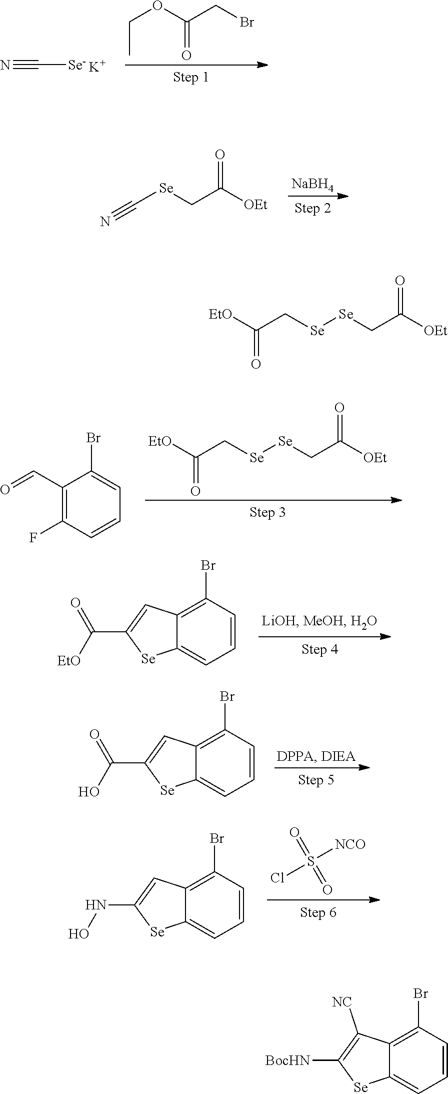

Steps 1 and 2: Synthesis of diethyl 2,2′-diselanediyldiacetate

Two reactions were performed in parallel as follows. To a solution of potassium selenocyanate (200 g, 1.39 mol) in EtOH (2000 mL) was added ethyl 2-bromoacetate (153 mL, 1.39 mol) and the reaction mixture was stirred at 80° C. for 2 h. The mixture was filtered, cooled to 0° C. and NaBH₄ (13.7 g, 363 mmol) was added. The mixture was stirred at 0° C. for 2 h. The reaction was quenched with H₂O (10 mL) at 0° C. and the mixture concentrated in vacuo. The combined residues of the two reactions were purified by silica gel chromatography (ISCO®; SepaFlash® Column, 0-10% EtOAc/PE gradient @ 100 mL/min) to give the title compound (270 g, 26% over two steps) as a yellow oil. LCMS m/z=335 [M+H]+

Step 3: Synthesis of ethyl 4-bromobenzo[b]selenophene-2-carboxylate

To a solution of 2-bromo-6-fluorobenzaldehyde (100 g, 379 mmol) in DMF (1 L) was added dithiothreitol (58.4 g, 379 mmol), the title compound of step 2 (209 g, 568 mmol) and K₂CO₃ (104 g, 757 mmol) and the reaction mixture was stirred at 60° C. for 1.5 h. The reaction mixture was concentrated in vacuo and the residue was purified by column chromatography (SiO₂, PE/EtOAc=100/1 to 10/1) to give the title compound (80.0 g, 59.0%) as an off-white solid. LCMS m/z=333 [M+H]+

Step 4: Synthesis of 4-bromobenzo[b]selenophene-2-carboxylic acid

To a solution of the title compound of step 3 (80.0 g, 241 mmol) in H₂O (400 mL) and MeOH (400 mL) was added LiOH·H₂O (30.3 g, 722 mmol) and the reaction mixture was stirred at 50° C. for 2 h. The reaction mixture was concentrated in vacuo, the residue acidified to pH 2 with 1M HCl and the mixture filtered. The solid was freeze-dried to give the title compound as an off-white solid, 55 g, crude. ¹H NMR (400 MHz, DMSO-d₆)=8.25 (s, 1H), 8.17 (d, 1H), 7.69 (d, 1H), 7.37 (t, 1H)

Step 5: Synthesis of tert-butyl (4-bromobenzo[b]selenophen-2-yl)carbamate

To a solution of the title compound of step 4 (45.0 g, 148 mmol) in t-BuOH (900 mL) and toluene (900 mL) was added DIEA (51.6 mL, 296 mmol) and DPPA (57.4 g, 208 mmol). The mixture was stirred at 90° C. for 3 h under N₂. The reaction mixture was evaporated under reduced pressure to give the title compound (27.0 g, crude) as a yellow solid. LCMS m/z=320 [M+H]+

Step 6: Synthesis of tert-butyl (4-bromo-3-cyanobenzo[b]selenophen-2-yl)carbamate

To a solution of the title compound of step 5 (22.0 g, 58.6 mmol) in MeCN (110 mL) was added dropwise sulfurisocyanatidic chloride (7.66 mL, 87.9 mmol) at −10° C. under N₂. The reaction mixture was stirred at −10° C. for 1.5 h, then, DMF (110 mL) was slowly added, and the mixture was stirred at −10° C. for 15 min. The mixture was filtered and the resulting solid dried to give the title compound, (28.5 g, 73.0%). LCMS: m/z=399, 401 [M+H]⁺

### Intermediate 22

### (S)-1-((S)-1-(methyl-d3)pyrrolidin-2-yl)ethan-1-ol

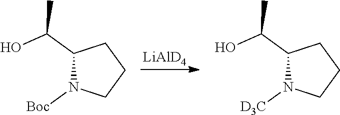

Two reactions were performed in parallel as follows. A solution of tert-butyl (S)-2-((S)-1-hydroxyethyl)pyrrolidine-1-carboxylate (65.0 g, 302 mmol) in THF (420 mL) was pumped at 0.778 mL/min to a flow reactor (PFA, Coils reactor, 3.175 (⅛″) mm, 15 mL, 60° C.). A solution of LiAlD₄, (2.0 M in THF, 453 mL) was pumped at 0.722 mL/min to the flow reactor. The residence time of flow reactor was 10 min. The mixture was collected with a bottle over 3 h. The reaction mixture was cooled to 0° C. and H₂O (35 mL) was added dropwise. 15% NaOH solution (103 mL) was then added drop-wise, followed by additional H₂O (35 mL) and the mixture was filtered. The combined filtrate's of the two combined reactions were evaporated under reduced pressure to give the title compound as an oil, 52 g. ¹H NMR (400 MHz, CDCl₃) δ=3.38 (quin, 1H), 3.02 (ddd, 1H), 2.26-2.44 (m, 2H), 1.83-1.96 (m, 1H), 1.64-1.81 (m, 2H), 1.49 (ddt, 1H), 1.14 (d, 3H)

### Intermediate 23

### tert-butyl (4-(5-chloro-1-(4-chloro-7,8-dihydro-5H-pyrano[4,3-d]pyrimidin-2-yl)-1H-indazol-4-yl)-3-cyano-7-fluorobenzo[b]thiophen-2-yl)carbamate

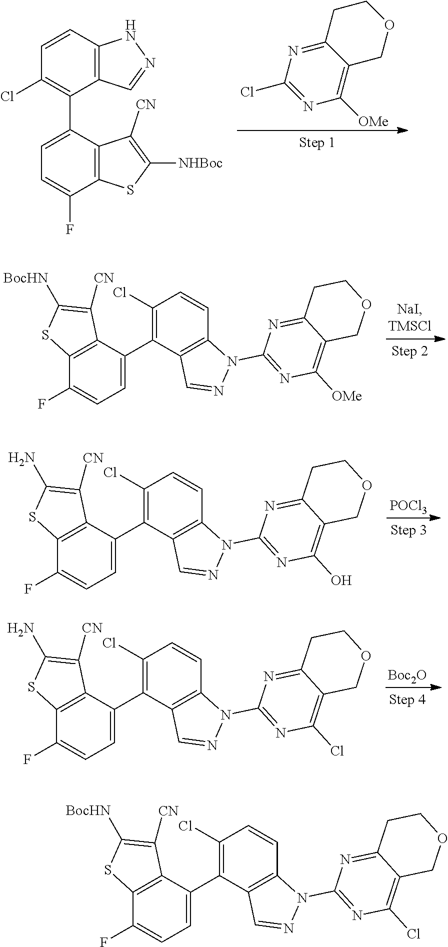

Step 1: Synthesis of tert-butyl (4-(5-chloro-1-(4-methoxy-7,8-dihydro-5H-pyrano[4,3-d]pyrimidin-2-yl)-1H-indazol-4-yl)-3-cyano-7-fluorobenzo[b]thiophen-2-yl)carbamate

Ephos Pd G4 (15.4 g, 16.7 mmol), Cs₂CO₃ (109 g, 334 mmol) and 2-chloro-4-methoxy-7,8-dihydro-5H-pyrano[4,3-d]pyrimidine (33.5 g, 167 mol) were added to a solution Intermediate 16 (74.0 g, 167 mmol) in dioxane (0.74 L) and the reaction mixture was stirred at 90° C. for 2 h under N₂. The mixture was poured into water (1.50 L) and extracted with DCM (2.00 L×2). The combined organic extracts were washed with brine (1.5 L×2), dried over Na₂SO₄, filtered and concentrated in vacuo. The crude product was triturated with PE (300 mL) at 25° C. for 30 min, the mixture filtered and the solid dried to give the title compound as a yellow solid, 86 g, 63%. LCMS m/z=607 [M+H]+

Step 2: Synthesis of 2-amino-4-(5-chloro-1-(4-hydroxy-7,8-dihydro-5H-pyrano[4,3-d]pyrimidin-2-yl)-1H-indazol-4-yl)-7-fluorobenzo[b]thiophene-3-carbonitrile

NaI (19.0 g, 175 mmol), TMSCl (26.2 g, 175 mmol) and MeCN (0.85 L) were added to the title compound of step 1 (85 g, 140 mmol) and the reaction mixture was stirred at 85° C. for 2 h. The reaction was poured into water (1.5 L) and the mixture extracted with EtOAc (1.5 L×2). The combined organic extracts were washed with brine (1.5 L), dried over Na₂SO₄, filtered and evaporated under reduced pressure to afford the title compound, 76.5 g, crude as a yellow solid. LCMS m/z=593 [M+H]+

Step 3: Synthesis of 2-amino-4-(5-chloro-1-(4-chloro-7,8-dihydro-5H-pyrano[4,3-d]pyrimidin-2-yl)-1H-indazol-4-yl)-7-fluorobenzo[b]thiophene-3-carbonitrile

POCl₃ (191 g, 1.25 mol) was added to a solution of the title compound of step 2 (70 g, 118 mmol) in toluene (700 mL) and the reaction mixture was stirred at 100° C. for 2 h. The reaction was cooled to 25° C., quenched with water (1.5 L) and the pH adjusted to 8 using aq. Na₂CO₃ solution. The mixture was extracted with EtOAc (1.5 L×2) and the combined organic extracts were washed with brine (2 L×2), dried over Na₂SO₄ and filtered. The filtrate was evaporated under reduced pressure to give the title compound as a brown solid, 55 g, 65%. LCMS m/z=511 [M+H]+

Step 4: Synthesis of tert-butyl (4-(5-chloro-1-(4-chloro-7,8-dihydro-5H-pyrano[4,3-d]pyrimidin-2-yl)-1H-indazol-4-yl)-3-cyano-7-fluorobenzo[b]thiophen-2-yl)carbamate

Boc₂O (38.4 g, 176 mmol), followed by TEA (17.8 g, 176 mmol) and DMAP (5.38 g, 44.0 mmol) were added to a solution of the title compound from step 3 (45 g, 88 mmol) in DCM (450 mL) and the reaction mixture was stirred at rt for 2 h. The reaction was poured into water (500 mL) and the mixture extracted with EtOAc (450 mL×2). The combined organic extracts were washed with brine (450 mL), dried over Na₂SO₄ and filtered. The filtrate was concentrated in vacuo and the crude product was triturated with DCM (120 mL) at rt for 30 mins. The mixture was filtered, the solid washed with DCM and dried in vacuo at 40° C. to give the title compound as an off-white solid, 27.5 g, 50.2%. LCMS m/z=611.0 [M+H]⁺

### Intermediate 24

### tert-butyl(S)-(4-(5-chloro-3-fluoro-6-methyl-1H-indazol-4-yl)-3-cyano-7-fluorobenzo[b]thiophen-2-yl)carbamate

### And Intermediate 25

### tert-butyl (R)-(4-(5-chloro-3-fluoro-6-methyl-1H-indazol-4-yl)-3-cyano-7-fluorobenzo[b]thiophen-2-yl)carbamate

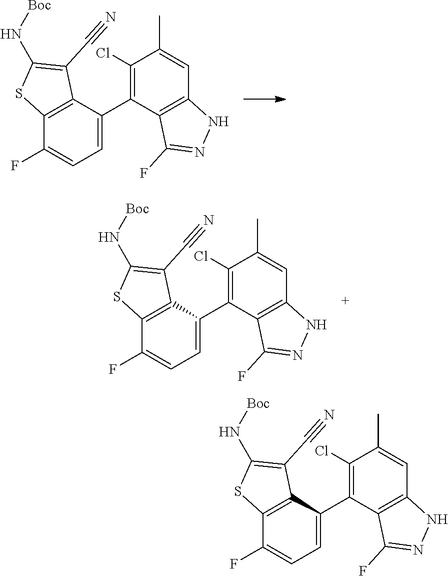

The title compound from Example 27 and 28, step 6 (150 g, 318 mmol) was separated by SFC (column: DAICEL CHIRALCEL OD (250 mm*50 mm, 10 μm); mobile phase: [CO2-MeOH(0.2% NH3H2O)]; B %: 40%, isocratic elution mode) to give Peak 1, Intermediate 24 (73 g, 150 mmol, 48% yield, Rt=1.351) as a yellow solid and Peak 2, Intermediate 25 (72 g, 150 mmol, 48% yield, Rt=1.470) as a yellow solid.

Intermediate 24, LCMS m/z=475.2 [M+H]⁺, ¹H NMR (400 MHz, CDCl₃) δ=9.56 (s, 1H), 7.95 (s, 1H), 7.39-7.30 (m, 2H), 7.20-7.09 (m, 1H), 2.59 (s, 3H), 1.56 (s, 9H).

Intermediate 25, LCMS m/z=475.2 [M+H]⁺, ¹H NMR (400 MHz, CDCl₃) δ=9.60 (s, 1H), 7.95 (s, 1H), 7.39-7.29 (m, 2H), 7.15 (dd, 1H), 2.59 (s, 3H), 1.56 (s, 9H).

The absolute configuration of Intermediate 25 was determined to be

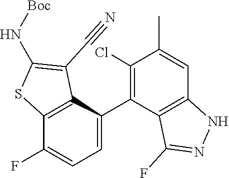

by micro-crystal electron diffraction (MicroED) using methods established by Jones et al. ACS.Cent. Sci. 2018, 4, 1587-1592. Experimental procedures were as follows:

A sample of material was spread over a holey carbon EM grid for data collection in a Thermo Fisher Scientific Talos F200C electron microscope at cryogenic temperature (~100 K). Crystals were illuminated with a parallel electron beam in NanoProbe mode. The total rotation angle per micro-crystal was 80-110° with an exposure time of 0.5 seconds. Data sets from 5 crystals were solved independently. The correct absolute structure out of the two possible enantiomorphs was then determined through dynamical refinement in PETS2 and Jana2020. The five datasets were individually refined against the two potential absolute structures, and each confirmed the same enantiomorph.

### Intermediate 26

### 2-chloro-4-((S)-1-((S)-1-methylpyrrolidin-2-yl)ethoxy)-6,7-dihydro-9H-[1,4]dioxepino[6,5-d]pyrimidine

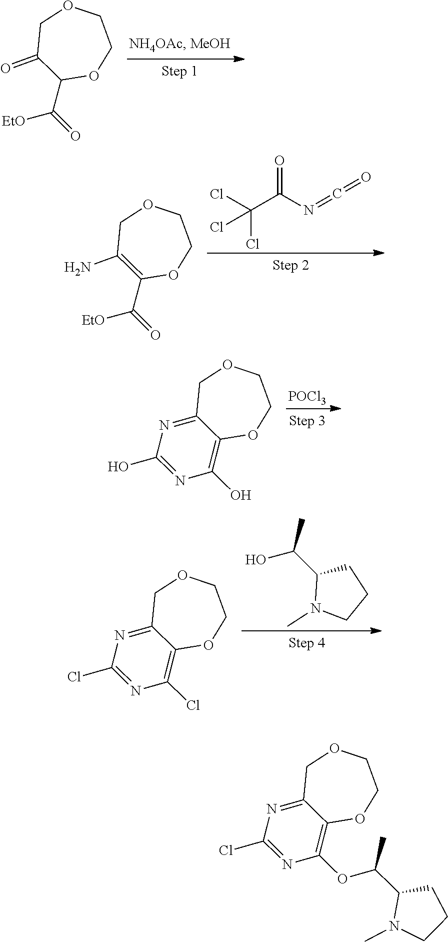

Step 1: Synthesis of ethyl 6-amino-2,3-dihydro-5H-1,4-dioxepine-7-carboxylate

To a stirred solution of ethyl 6-oxo-1,4-dioxepane-5-carboxylate (184 g, 977 mmol) in MeOH (1.8 L) was added NH₄OAc (452 g, 5.87 mmol) and the reaction was stirred for 16 h at 45-50° C. The reaction mixture was cooled, concentrated in vacuo, diluted in H₂O (1.5 L) then extracted in EtOAc (3×1.0 L). The combined organic layers were dried over anhydrous Na₂SO₄, filtered and concentrated to afford a crude residue.

Step 2: Synthesis of 6,7-dihydro-9H-[1,4]dioxepino[6,5-d]pyrimidine-2,4-diol

To a solution of the crude residue from step 1 in MeCN (1.6 L) cooled to 0-5° C. was added 2,2,2-trichloroacetyl isocyanate (179 g, 953 mmol) at 0° C. and the solution stirred for 0.5 h at rt. The mixture was then concentrated in vacuo and treated with NH₃·MeOH (7M, 259 mL) and the reaction mixture was stirred at 40-45° C. for 0.5 h. The reaction mixture was concentrated in vacuo and the residue was triturated with EtOH (1.0 L) to give the title compound 70.0 g, 68% over two steps) as a yellow solid. LCMS: m/z=185 [M+H]⁺.

Step 3: Synthesis of 2,4-dichloro-6,7-dihydro-9H-[1,4]dioxepino[6,5-d]pyrimidine

To a stirred solution of the title compound of step 2 (70.0 g, 380 mmol) in dioxane (700 mL) at rt was treated with POCl₃ (177 mL) and the reaction mixture was stirred for 16 h at 100° C. The cooled mixture was concentrated and slowly poured into H₂O (1.40 L) stirring at rt for 0.5 h then extracted with EtOAc (3×700 ml). The combined organic layers were dried over anhydrous Na₂SO₄ and concentrated. The residue was purified by silica gel column with Hexanes:EtOAc=1:0-5:1. The residue was then triturated with hexanes: EtOAc 20:1 to give the title compound (7.5 g, 8.9%) as an off-white solid. LCMS: m/z=221 [M+H]⁺.

Step 4: Synthesis of 2-chloro-4-((S)-1-((S)-1-methylpyrrolidin-2-yl)ethoxy)-6,7-dihydro-9H-[1,4]dioxepino[6,5-d]pyrimidine

To a stirred solution of (S)-1-((S)-1-methylpyrrolidin-2-yl)ethan-1-ol (4.0 g, 31.0 mmol) in toluene (80 ml) was added potassium tert-butoxide (4.06 g, 36.2 mmol) at 25° C. After stirring for 10 min, a solution of the title compound from step 3 (4.0 g, 18.1 mmol) in toluene (40 ml) was added at rt and stirred for 3 h. The reaction mixture was diluted in water (150 mL) and then extracted with EtOAc (3×100 mL). The combined organic layers were dried over anhydrous Na₂SO₄ and concentrated. The residue was purified by silica gel column with (PE:EtOAc=1:0 to 0:1). to give the title compound as a yellow oil (3.93 g, 68%). LCMS: m/z=314.1 [M+H]⁺.

### Intermediate 27

### 2-chloro-4-((S)-1-((S)-1-methylpyrrolidin-2-yl)ethoxy)-5,6,8,9-tetrahydro-7H-pyrimido[4,5-c]azepin-7-one

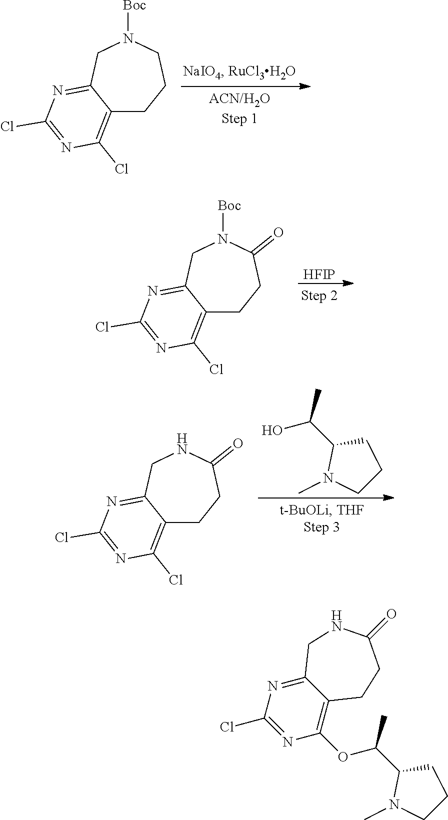

Step 1: Synthesis of tert-butyl 2,4-dichloro-7-oxo-5,6,7,9-tetrahydro-8H-pyrimido[4,5-c]azepine-8-carboxylate

To a stirred solution of tert-butyl 2,4-dichloro-5,6,7,9-tetrahydro-8H-pyrimido[4,5-c]azepine-8-carboxylate (1.07 g, 3.36 mmol) in ACN (10.7 ml) and water (21.4 ml) was added sodium periodate (2.16 g, 10.1 mmol, 559.00 μL) and trichlororuthenium; trihydrate (132 mg, 504 μmol). The mixture was stirred at rt over 1 h then diluted with water (10.0 mL), quenched with aqueous saturated Na₂SO₃ (20 mL) and extracted with EtOAc (3×30 mL). The combined organic layers were washed with brine (2×20 mL), dried over Na₂SO₄, filtered and concentrated under reduced pressure. The residue was purified by column chromatography (SiO₂, hexanes:EtOAc=20/1) to afford the title compound (0.64 g, 1.83 mmol, 54.4% yield) as white solid. ¹H NMR (400 MHz, CDCl₃): δ 5.11 (s, 2H), 3.20-3.14 (m, 2H), 3.12-3.05 (m, 2H), 1.52 (s, 9H)

Step 2: Synthesis of 2,4-dichloro-5,6,8,9-tetrahydro-7H-pyrimido[4,5-c]azepin-7-one

A solution of the title compound from step 1 (500 mg, 1.51 mmol) in HFIP (10.0 ml) was stirred at 70° C. for 12 h. The mixture was concentrated under reduced pressure, and the residue was purified by column chromatography (SiO₂, hexanes:EtOAc=1:1 to 0:1) to afford the title compound (270 mg, 1.16 mmol, 77.3% yield) as white solid. ¹H NMR (400 MHz, 6d-DMSO): δ 8.03 (t, 1H), 4.44 (d, 2H), 3.00-2.90 (m, 2H), 2.83-2.75 (m, 2H).

Step 3: Synthesis of 2-chloro-4-((S)-1-((S)-1-methylpyrrolidin-2-yl)ethoxy)-5,6,8,9-tetrahydro-7H-pyrimido[4,5-c]azepin-7-one

To a stirred solution of (S)-1-((S)-1-methylpyrrolidin-2-yl)ethan-1-ol (55.7 mg, 431 μmol) in THF (4.0 ml) was added lithium tert-butoxide (2.2 M, 294 μL) at 25° C. After stirring for 0.5 h, the title compound from step 2 (2.2 M, 294 μL) was added at rt and stirred at 30° C. for 12 h. The reaction mixture was quenched by the addition of HCl (1M aqueous) adjusting the pH to 6. The resulting mixture was purified by prep-HPLC (column: Phenomenex Luna C18 150*25 mm*10 μm; mobile phase: [H2O(0.225% FA)-ACN]; gradient: 0%-25% B over 12.0 min) to afford the title compound (20 mg, 61.0 μmol, 14.2% yield) as a yellow solid.

### Intermediate 28

### 2-chloro-4-((S)-1-((2S,4R)-1,4-dimethylpyrrolidin-2-yl)ethoxy)-6,7-dihydro-9H-[1,4]dioxepino[6,5-d]pyrimidine

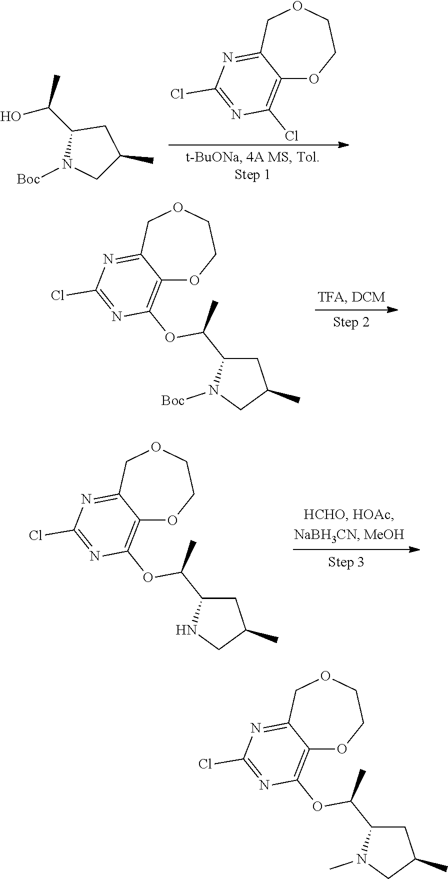 [SMILES: C[C@@H]1C[C@@H]([C@H](C)O)N(C(=O)OC(C)(C)C)C1 ; Clc1nc(Cl)c2c(n1)COCCO2 ; C[C@@H]1C[C@@H]([C@H](C)Oc2nc(Cl)nc3c2OCCOC3)N(C(=O)OC(C)(C)C)C1 ; C[C@H]1CN[C@H]([C@H](C)Oc2nc(Cl)nc3c2OCCOC3)C1 ; C[C@@H]1C[C@@H]([C@H](C)Oc2nc(Cl)nc3c2OCCOC3)N(C)C1]

Step 1: Synthesis of tert-butyl(2S,4R)-2-((S)-1-((2-chloro-6,7-dihydro-9H-[1,4]dioxepino[6,5-d]pyrimidin-4-yl)oxy)ethyl)-4-methylpyrrolidine-1-carboxylate

To a stirred solution of tert-butyl (2S,4R)-2-((S)-1-hydroxyethyl)-4-methylpyrrolidine-1-carboxylate (250 mg, 1.09 mmol) and the title compound from step 3 Intermediate 26 (241 mg, 1.09 mmol) in toluene (5 mL) was added sodium tert-butoxide (157.2 mg, 1.64 mmol) and molecular sieves (250 mg) at 25° C. The mixture was stirred for 1 h, diluted with water (10 mL) and extracted with EtOAc (3×10 mL). The combined organic layers were washed with brine (3×10 mL), dried over Na₂SO₄, filtered and concentrated under reduced pressure. The residue was purified by column chromatography (SiO₂, hexanes:EtOAc=100:1 to 3:1) to give the title compound (450 mg, 1.09 mmol, 99% yield) as a colorless oil. LCMS data: M−100+1 (314.1)

Step 2: Synthesis of 2-chloro-4-((S)-1-((2S,4R)-4-methylpyrrolidin-2-yl)ethoxy)-6,7-dihydro-9H-[1,4]dioxepino[6,5-d]pyrimidine

A solution of the title compound from step 1 (400 mg, 966 μmol) in DCM (6.0 mL) was added TFA (3.07 g, 26.9 mmol, 2.00 mL) and stirred at 25° C. for 1 h. The mixture was concentrated under reduced pressure to give the title compound (410 mg, 958 μmol, 99% yield, TFA) as a yellow oil.

Step 3: Synthesis of 2-chloro-4-((S)-1-((2S,4R)-1,4-dimethylpyrrolidin-2-yl)ethoxy)-6,7-dihydro-9H-[1,4]dioxepino[6,5-d]pyrimidine

To a solution of the title compound from step 2 (410 mg, 958 μmol, TFA) and formaldehyde (446 mg, 5.51 mmol, 410 μL) in methanol (8 mL) was added acetic acid (215 mg, 3.58 mmol, 205 μL) and it was stirred at 25° C. for 0.5 h. To the mixture was added sodium cyanoborohydride (301 mg, 4.79 mmol) and it was stirred at 25° C. for 0.5 h then concentrated under reduced pressure. The crude product was purified by reverse-phase HPLC (0.1% FA condition) to give the title compound (350 mg, 883 μmol, 92% yield, FA) as a colorless oil. LCMS m/z=328.2 [M+H]+

### Intermediate 29

### 2′-chloro-4′-((S)-1-((S)-1-methylpyrrolidin-2-yl)ethoxy)-2,3,5,6-tetrahydro-5′H,7′H-spiro[pyran-4,8′-pyrano[4,3-d]pyrimidine]

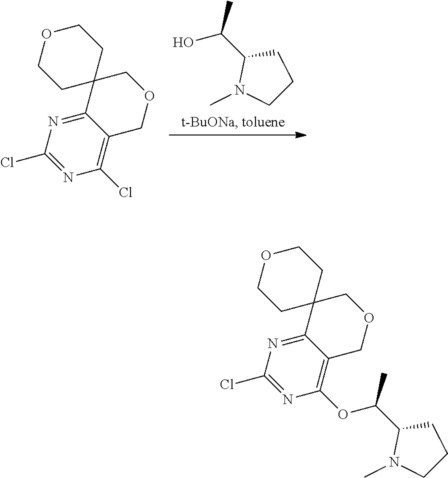

To a mixture of 2′,4′-dichloro-2,3,5,6-tetrahydro-5′H,7′H-spiro[pyran-4,8′-pyrano[4,3-d]pyrimidine] (180 mg, 654 μmol) and (S)-1-((S)-1-methylpyrrolidin-2-yl)ethan-1-ol (101 mg, 785 μmol) in toluene (3 mL) was added sodium tert-butoxide (125 mg, 1.31 mmol), the reaction mixture was stirred at 25° C. for 0.5 h. The reaction mixture was diluted with water (10 ml) and extracted with EtOAc (2×10 mL). The combined organic layers were washed with brine (10 mL), dried over Na₂SO₄, filtered and concentrated under reduced pressure to give a residue. The crude product was purified by reverse-phase HPLC (0.1% FA condition) to give the title compound (240 mg, 98% yield) as a colorless oil. LCMS m/z=368.2 [M+H]+

### Intermediate 30

### 2-chloro-4-((S)-1-((S)-1-methylpyrrolidin-2-yl)ethoxy)-7,8-dihydro-5H-pyrano[4,3-d]pyrimidine

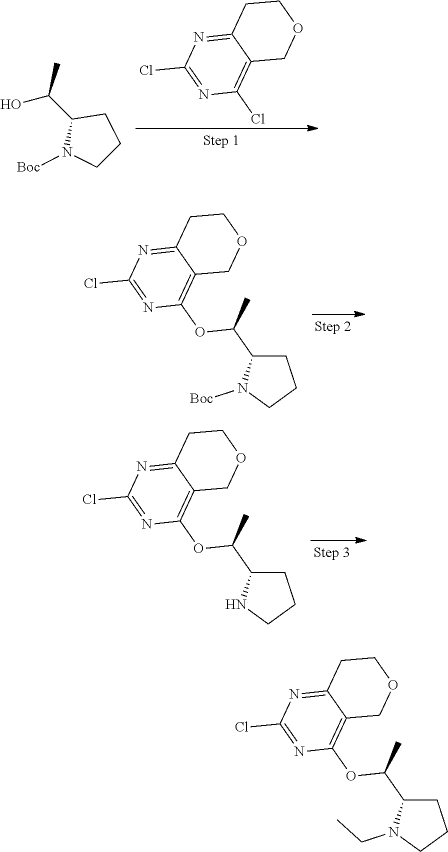

Step 1: Synthesis of tert-butyl(S)-2-((S)-1-((2-chloro-7,8-dihydro-5H-pyrano[4,3-d]pyrimidin-4-yl)oxy)ethyl)pyrrolidine-1-carboxylate

To a stirred solution of tert-butyl(S)-2-((S)-1-hydroxyethyl)pyrrolidine-1-carboxylate (647 mg, 3.0 mmol) and 2,4-dichloro-7,8-dihydro-5H-pyrano[4,3-d]pyrimidine (684 mg, 3.30 mmol) in THF (12.0 mL) cooled to 0° C. was added sodium tert-butoxide (1.8 mL, 2.0M in THF). After 15 min at 0° C. the mixture was stirred at rt for 2 h. The mixture was quenched with saturated. aq. NH₄Cl diluted with water (10 ml) and extracted with EtOAc (3×40 mL). The combined organic layers were washed with brine, dried over Na₂SO₄, filtered and concentrated under reduced pressure. The residue was purified by column chromatography (SiO2, 0-80% hexanes:EtOAc) to give a 2:1 mixture of the desired product and a hydroxy pyrimidine byproduct compound as a colorless oil. The material was carried forward (450 mg, 99% yield) as a colorless oil. LCMS data: M−100+1 (314.1)

Step 2: Synthesis of 2-chloro-4-((S)-1-((S)-pyrrolidin-2-yl)ethoxy)-7,8-dihydro-5H-pyrano[4,3-d]pyrimidine

A solution of the mixture from step 1 (693 mg) in DCM (6.0 ml) was added TFA (1.5 mL) and stirred at 30° C. for 1 h. The mixture was concentrated under reduced pressure and azeotroped with toluene to afford a residue that was carried forward without further purification.

Step 3: Synthesis of 2-chloro-4-((S)-1-((S)-1-methylpyrrolidin-2-yl)ethoxy)-7,8-dihydro-5H-pyrano[4,3-d]pyrimidine

To a solution of the crude material from step 2 in THF (10 ml) was added TEA (0.38 mL) and acetaldehyde (0.150 mL) and the mixture was stirred at rt for 10 min then treated with sodium triacetoxyborohydride (559 mg). The mixture was stirred at rt overnight then quenched with saturated. aq. NaHCO₃, extracted with EtOAc (4×40 mL). The combined organics were washed with brine then dried over Na₂SO₄, filtered and concentrated under reduced pressure. The crude product was purified by column chromatography (SiO2, MeOH:DCM=0-20%) to afford the title compound (118 mg) as yellow solid. ¹H NMR (500 MHz, CDCl₃) δ 5.37-5.20 (m, 1H), 4.57 (q, 2H), 3.98 (td, 2H), 3.12 (s, 1H), 2.95-2.80 (m, 3H), 2.80-2.69 (m, 1H), 2.40 (dt, 1H), 2.23 (q, 1H), 1.89-1.62 (m, 4H), 1.29 (d, 3H), 1.08 (t, 3H)

### Intermediate 31

### (S)-2-chloro-7-methyl-4-((S)-1-((S)-1-methylpyrrolidin-2-yl)ethoxy)-6,7-dihydro-9H-[1,4]dioxepino[6,5-d]pyrimidine

### And Intermediate 32

### (R)-2-chloro-6-methyl-4-((S)-1-((S)-1-methylpyrrolidin-2-yl)ethoxy)-6,7-dihydro-9H-[1,4]dioxepino[6,5-d]pyrimidine

### And Intermediate 33

### (S)-2-chloro-6-methyl-4-((S)-1-((S)-1-methylpyrrolidin-2-yl)ethoxy)-6,7-dihydro-9H-[1,4]dioxepino[6,5-d]pyrimidine

### And Intermediate 34

### (R)-2-chloro-7-methyl-4-((S)-1-((S)-1-methylpyrrolidin-2-yl)ethoxy)-6,7-dihydro-9H-[1,4]dioxepino[6,5-d]pyrimidine

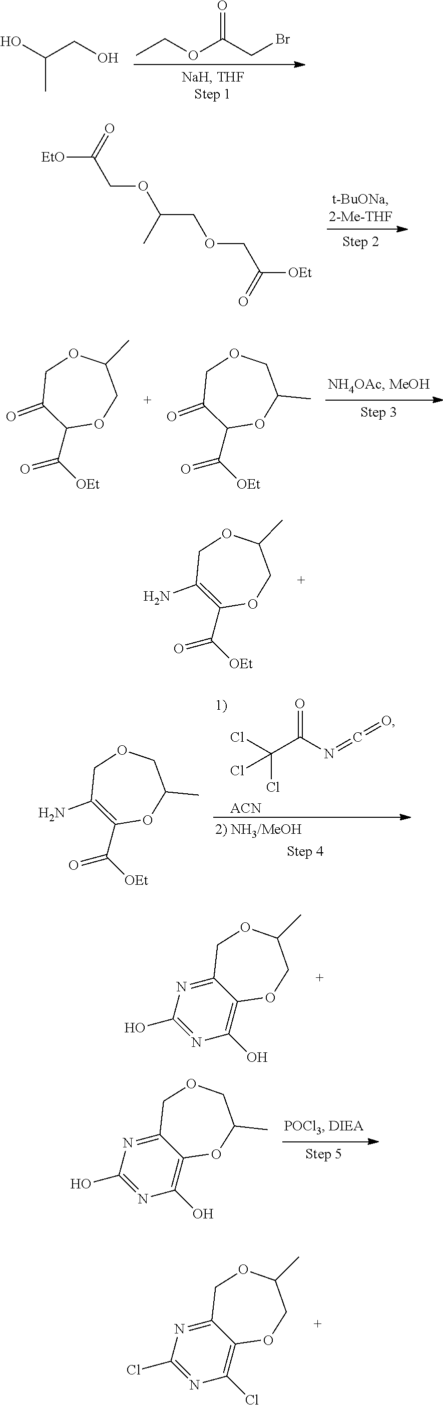

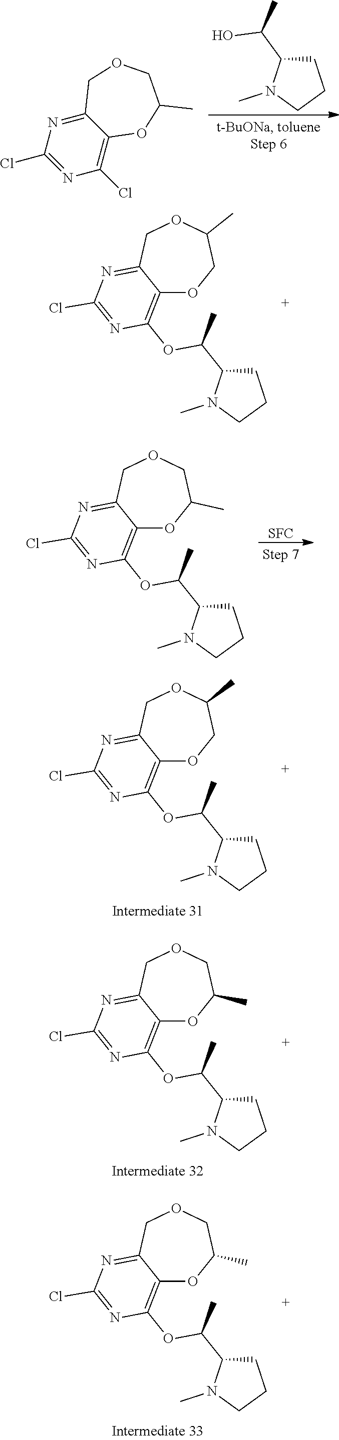

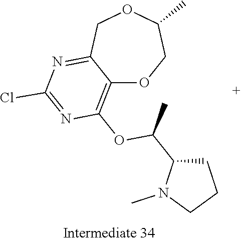 [SMILES: C[C@@H]1COc2c(nc(Cl)nc2O[C@@H](C)[C@@H]2CCCN2C)CO1]

Step 1: Synthesis of diethyl 2,2′-(propane-1,2-diylbis(oxy))diacetate

To a solution of propane-1,2-diol (20 g, 262 mmol) in THF (400 mL) was added sodium hydride, 60% in mineral oil (22.0 g, 551 mmol) at 0° C. under N₂ and was stirred at rt for 0.5 h. The mixture was then cooled to 0° C. and treated with ethyl 2-bromoacetate (89.9 g, 538 mmol), then warmed to rt and stirred for 11.5 h. The mixture was then quenched by addition of aqueous saturated. NH₄Cl solution (200 mL) at 0° C., diluted with water (200 mL) and extracted with EtOAc (2×250 mL). The combined organic layers were washed with brine (2×200 mL), dried over Na₂SO₄, filtered and concentrated under reduced pressure. The residue was purified by column chromatography (SiO₂, eluting with PE/EtOAc=40/1 to 1/1) to afford the title compound (14 g, 21.4% yield) as a yellow oil. ¹H NMR (400 MHz, CDCl₃) δ ppm 4.25-4.17 (m, 6H), 4.13 (d, J=8.0 Hz, 2H), 3.84-3.77 (m, 1H), 3.62-3.57 (m, 2H), 1.31-1.27 (m, 6H), 1.24-1.21 (m, 3H)

Step 2: Synthesis of ethyl 2-methyl-6-oxo-1,4-dioxepane-5-carboxylate and ethyl 3-methyl-6-oxo-1,4-dioxepane-5-carboxylate

A mixture of sodium tert-butoxide (10.2 g, 106 mmol) in 2-MeTHF (120 mL) was heated to 70° C. under N₂ then a solution of the title compound from step 1 (12 g, 48.3 mmol) in 2-methyltetrahydrofuran (30 mL) was added to the mixture slowly at 70° C. and stirred for 1 h, then cooled to 20° C. and stirred for 11 h. The mixture was acidified by addition of 6 M aqueous HCl (25 mL), diluted with water (60 mL) and extracted with EtOAc (3×100 mL). The combined organic layers were dried over Na₂SO₄, filtered and concentrated under reduced pressure. The residue was purified by column chromatography (SiO₂ eluting with PE/EtOAc=40/1 to 1/1) to afford a mixture of ethyl 2-methyl-6-oxo-1,4-dioxepane-5-carboxylate and ethyl 3-methyl-6-oxo-1,4-dioxepane-5-carboxylate as a yellow oil. ¹H NMR (400 MHz, CDCl₃) δ ppm 4.99-4.82 (m, 1H), 4.39-4.07 (m, 5H), 4.03-3.80 (m, 1H), 3.56-3.33 (m, 1H), 1.36-1.28 (m, 3H), 1.26-1.17 (m, 3H).

Step 3: Synthesis of ethyl 6-amino-3-methyl-2,3-dihydro-5H-1,4-dioxepine-7-carboxylate and ethyl 6-amino-2-methyl-2,3-dihydro-5H-1,4-dioxepine-7-carboxylate

To a solution of the mixture from step 2 (1.15 g, 5.69 mmol) in MeOH (30 mL) was added NH₄OAc (2.63 g, 34.1 mmol) at rt and stirred for 12 h. The mixture was partitioned between DCM (100 mL) and water (50 mL). The organic phase was separated, washed with brine (2×50 mL), dried over Na₂SO₄, filtered and concentrated under reduced pressure to give a mixture of ethyl 6-amino-3-methyl-2,3-dihydro-5H-1,4-dioxepine-7-carboxylate and ethyl 6-amino-2-methyl-2,3-dihydro-5H-1,4-dioxepine-7-carboxylate (1.14 g, crude) as a yellow gum.

Step 4: Synthesis of 7-methyl-6,7-dihydro-9H-[1,4]dioxepino[6,5-d]pyrimidine-2,4-diol and 6-methyl-6,7-dihydro-9H-[1,4]dioxepino[6,5-d]pyrimidine-2,4-diol

To a solution of the crude mixture from step 3 (1.14 g, 5.67 mmol) in ACN (25 mL) was added 2,2,2-trichloroacetyl isocyanate (2.35 g, 12.4 mmol) at rt and the mixture was stirred for 0.5 h. The resultant precipitate was collected by filtration, dissolved in NH₃ in MeOH solution (7 M, 22.9 mL) then heated at 70° C. for 8 h. The mixture was concentrated under reduced pressure then triturated with PE:EtOAc=1:3 (4 mL) to afford a mixture of the title compounds (0.7 g, 62.3% yield) as a yellow solid. LCMS m/z=199.0 [M+H]⁺, ¹H NMR (400 MHz, 6d-DMSO) δ ppm 10.65-8.87 (m, 2H), 4.60-4.31 (m, 2H), 4.20-3.76 (m, 2H), 3.57-3.41 (m, 1H), 1.24-0.95 (m, 3H)

Step 5: Synthesis of 2,4-dichloro-7-methyl-6,7-dihydro-9H-[1,4]dioxepino[6,5-d]pyrimidine and 2,4-dichloro-6-methyl-6,7-dihydro-9H-[1,4]dioxepino[6,5-d]pyrimidine

To the mixture from step 4 (700 mg, 3.53 mmol) in POCl₃ (30 mL) was added DIEA (2.28 g, 17.6 mmol) at rt then stirred at 110° C. for 12 h under N₂. The mixture was poured into ice water (300 mL) and extracted with EtOAc (3×200 mL). The combined organic layers were washed with brine (3×300 mL), dried over Na₂SO₄, filtered, concentrated under reduced pressure then purified by column chromatography (SiO₂, PE/EtOAc=40/1 to 1/1) to give a mixture of the title compounds (0.65 g, 75.9% yield) as a yellow gum. LCMS m/z=235.1 [M+H]⁺, ¹H NMR (400 MHz, CDCl₃) δ ppm 4.93-4.67 (m, 2H), 4.50-4.19 (m, 1H), 4.17-4.02 (m, 1H), 3.82-3.68 (m, 1H), 1.45-1.22 (m, 3H).

Step 6: Synthesis of 2-chloro-7-methyl-4-((S)-1-((S)-1-methylpyrrolidin-2-yl)ethoxy)-6,7-dihydro-9H-[1,4]dioxepino[6,5-d]pyrimidine and 2-chloro-6-methyl-4-((S)-1-((S)-1-methylpyrrolidin-2-yl)ethoxy)-6,7-dihydro-9H-[1,4]dioxepino[6,5-d]pyrimidine

To a solution of the mixture from step 5 (0.5 g, 2.13 mmol) in toluene (20 mL) was added(S)-1-((S)-1-methylpyrrolidin-2-yl)ethan-1-ol, 4A molecular sieve (200 mg) and sodium tert-butoxide (817 mg, 8.51 mmol) at 0° C. The mixture was stirred at 0° C. for 0.5 h then partitioned between EtOAc (200 mL) and water (50 mL). The organic phase was separated, washed with brine (2×50 mL), dried over Na₂SO₄, filtered and concentrated under reduced pressure to give a crude product was purified by reversed-phase HPLC (0.1% formic acid condition) to give a mixture of the title compounds (0.65 g, 76.8% yield) as a yellow gum. LCMS m/z=328.2 [M+H]+

Step 7: Synthesis of (S)-2-chloro-7-methyl-4-((S)-1-((S)-1-methylpyrrolidin-2-yl)ethoxy)-6,7-dihydro-9H-[1,4]dioxepino[6,5-d]pyrimidine and (R)-2-chloro-6-methyl-4-((S)-1-((S)-1-methylpyrrolidin-2-yl)ethoxy)-6,7-dihydro-9H-[1,4]dioxepino[6,5-d]pyrimidine and (S)-2-chloro-6-methyl-4-((S)-1-((S)-1-methylpyrrolidin-2-yl)ethoxy)-6,7-dihydro-9H-[1,4]dioxepino[6,5-d]pyrimidine and (R)-2-chloro-7-methyl-4-((S)-1-((S)-1-methylpyrrolidin-2-yl)ethoxy)-6,7-dihydro-9H-[1,4]dioxepino[6,5-d]pyrimidine

The mixture from step 7 was separated by chiral SFC (column: DAICEL CHIRALPAK AD (250 mm*50 mm, 10 μm); mobile phase: [carbon dioxide-isopropanol (0.1% ammonium hydroxide)]; B %: 20%, isocratic elution mode) to give three fractions. The first fraction was further separated by SFC (column: DAICEL CHIRALPAK IG (250 mm*50 mm, 10 μm); mobile phase: [Heptane-ethanol (0.1% trifluoroacetic acid)]; B %: 2%, isocratic elution mode) to give Intermediate 31 (S)-2-chloro-7-methyl-4-((S)-1-((S)-1-methylpyrrolidin-2-yl)ethoxy)-6,7-dihydro-9H-[1,4]dioxepino[6,5-d]pyrimidine as a yellow gum, LCMS m/z=328.1 [M+H]⁺, ¹H NMR (400 MHz, CDCl₃) δ ppm 5.31-5.15 (m, 1H), 4.83-4.73 (m, 1H), 4.72-4.63 (m, 1H), 4.42-4.25 (m, 1H), 4.09-3.91 (m, 1H), 3.73-3.57 (m, 1H), 3.14-3.02 (m, 1H), 2.71-2.57 (m, 1H), 2.47 (s, 3H), 2.34-2.22 (m, 1H), 1.97-1.87 (m, 1H), 1.77-1.65 (m, 3H), 1.35 (d, 3H), 1.23 (d, 3H), SFC showed 99% ee

and Intermediate 32 (R)-2-chloro-6-methyl-4-((S)-1-((S)-1-methylpyrrolidin-2-yl)ethoxy)-6,7-dihydro-9H-[1,4]dioxepino[6,5-d]pyrimidine as a yellow gum, LCMS m/z=328.1 [M+H]+, ¹H NMR (400 MHz, CDCl₃) δ ppm 5.23-5.12 (m, 1H), 4.79-4.70 (m, 1H), 4.67-4.58 (m, 1H), 4.16-4.06 (m, 1H), 4.01 (d, 1H), 3.73-3.62 (m, 1H), 3.08 (t, 1H), 2.67-2.57 (m, 1H), 2.49 (s, 3H), 2.34-2.24 (m, 1H), 1.95-1.88 (m, 1H), 1.81-1.67 (m, 3H), 1.35 (d, 3H), 1.31 (d, 3H), SFC showed 84% ee.

The second fraction afforded Intermediate 33 (S)-2-chloro-6-methyl-4-((S)-1-((S)-1-methylpyrrolidin-2-yl)ethoxy)-6,7-dihydro-9H-[1,4]dioxepino[6,5-d]pyrimidine as a yellow gum; LCMS m/z=328.1, ¹H NMR (400 MHz, CDCl₃) δ ppm 5.22-5.10 (m, 1H), 4.75-4.65 (m, 1H), 4.61-4.51 (m, 1H), 4.13-4.00 (m, 1H), 3.98-3.90 (m, 1H), 3.64-3.54 (m, 1H), 3.18-3.10 (m, 1H), 2.76-2.64 (m, 1H), 2.53 (s, 3H), 2.39-2.27 (m, 1H), 1.98-1.89 (m, 1H), 1.86-1.60 (m, 3H), 1.29 (d, 3H), 1.24 (d, 3H), SFC showed 99% ee.

The third fraction afforded Intermediate 34 (R)-2-chloro-7-methyl-4-((S)-1-((S)-1-methylpyrrolidin-2-yl)ethoxy)-6,7-dihydro-9H-[1,4]dioxepino[6,5-d]pyrimidine as a yellow gum, LCMS m/z=328.1, ¹H NMR (400 MHz, CDCl₃) δ ppm 5.32-5.22 (m, 1H), 4.85-4.63 (m, 2H), 4.32-4.21 (m, 1H), 4.05-3.93 (m, 1H), 3.67-3.57 (m, 1H), 3.30-3.23 (m, 1H), 2.86-2.77 (m, 1H), 2.62 (s, 3H), 2.50-2.38 (m, 1H), 2.05-1.97 (m, 1H), 1.93-1.70 (m, 3H), 1.37 (d, 3H), 1.20 (d, 3H), SFC showed 97% ee.

### Intermediate 35

### tert-butyl (4-(5-chloro-3-fluoro-6-methoxy-1H-indazol-4-yl)-3-cyano-7-fluorobenzo[b]thiophen-2-yl)carbamate

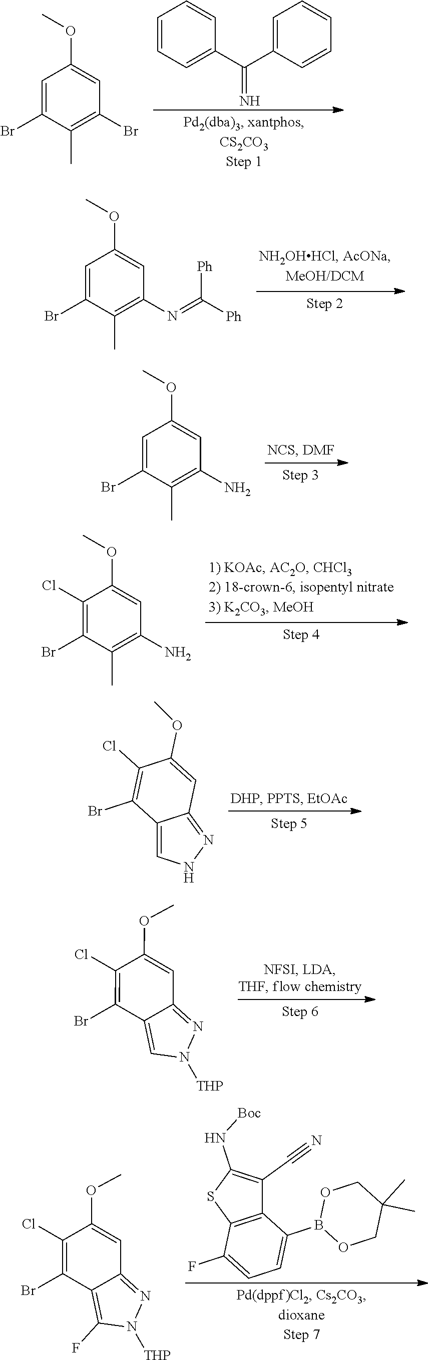

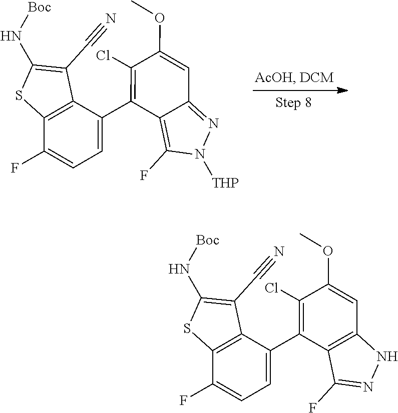

Step 1: Synthesis of N-(3-bromo-5-methoxy-2-methylphenyl)-1,1-diphenylmethanimine

A mixture of 1,3-dibromo-5-methoxy-2-methyl-benzene (100 g, 357 mmol, diphenylmethanimine (64.7 g, 357 mmol, 59.9 mL), 4,5-bis(diphenylphosphino)-9,9-dimethylxanthene (41.3 g, 71.4 mmol), tris(dibenzylideneacetone) dipalladium (0) (32.7 g, 35.7 mmol) and Cs₂CO₃ (349 g, 1.07 mol) in dioxane (500 mL) was degassed and purged with N₂ and the mixture was stirred at 100° C. for 5 h under N₂. The mixture was cooled to rt and filtered through Celite washing with EtOAc. The filtrate was concentrated under reduced pressure. The resultant residue was purified by column chromatography (SiO₂, PE/EtOAc=1/0, PE/EtOAc=10/1) to give the title compound (110 g, 76.3% yield) as a yellow solid. LCMS m/z=380.0 [M+H]+

Step 2: Synthesis of 3-bromo-5-methoxy-2-methylaniline

To a solution of the title compound from step 1 (110 g, 273 mmol in MeOH (612 mL) and DCM (612 mL) was added sodium acetate (157 g, 1.91 mol) and hydroxylamine hydrochloride (114 g, 1.64 mol) at rt. The mixture was then stirred at rt for 16 h and then concentrated in vacuo. The residue was diluted with water (600 mL) and extracted with EtOAc (3×600 mL). The combined organic layers were concentrated in vacuo. The residue was diluted with 1 N aqueous HCl (600 mL) to pH=1 and stirred at rt for 0.5 hr. The mixture was then extracted with EtOAc (2×600 mL). The pH of the aqueous layer was adjusted with aqueous 1 N NaOH solution to pH 8-9 then extracted with EtOAc (3×600 mL). The combined organics were washed with brine (600 mL), filtered and concentrated in vacuo to give the title compound (45.9 g, 77.4% yield) as a yellow solid. LCMS m/z=218.0 [M+H]+

Step 3: Synthesis of 3-bromo-4-chloro-5-methoxy-2-methylaniline

To a solution of the title compound from step 2 (45.9 g, 211 mmol) in DMF (420 mL) cooled to 0° C. was added NCS (31.0 g, 232 mmol) at 0° C., then the reaction mixture was stirred at rt for 5 h. The mixture was diluted with water (1 L), extracted with EtOAc (3×450 mL). The combined organic layers were washed with brine (500 mL), dried over Na₂SO₄, filtered and concentrated in vacuo. The crude product was purified by column chromatography (SiO₂ hexanes:EtOAc=100/1 to 5/1) to give the title compound (41.5 g, 78.3% yield) as a brown solid. LCMS m/z=250.0 [M+H]+

Step 4: Synthesis of 4-bromo-5-chloro-6-methoxy-2H-indazole

A mixture of the title compound from step 3 (41.5 g, 166 mmol), potassium acetate (19.5 g, 198 mmol), and acetic anhydride (67.6 g, 662 mmol, 62.2 mL) in chloroform (415 mL) was stirred at 70° C. for 1 h. 18-crown-6 (4.38 g, 16.5 mmol) and isopentyl nitrite (194 g, 1.66 mol, 223 mL) were added, then the mixture was stirred at 70° C. for 3 h. K₂CO₃ (68.6 g, 497 mmol) and MeOH (415 mL) were added and stirred at 60° C. for 16 h. The mixture was cooled then concentrated in vacuo, diluted with water (1.5 L), extracted with EtOAc (1.5 L×3). The combined organic layers were washed with brine (1.5 L), dried over Na₂SO₄, filtered and concentrated in vacuo. The crude product was triturated with n-hexane (1.0 L) at 25° C. for 30 min to give the title compound (25 g, 57.7% yield) as a brown solid. LCMS m/z=261.0 [M+H]+

Step 5: Synthesis of 4-bromo-5-chloro-6-methoxy-2-(tetrahydro-2H-pyran-2-yl)-2H-indazole

To a solution of the title compound from step 4 (25.0 g, 95.6 mmol) in EtOAc (250 mL) was added 3,4-dihydro-2H-pyran (24.1 g, 286 mmol) and PPTS (2.4 g, 9.56 mmol) at rt under N₂, the mixture was stirred at 30-35° C. for 16 h. The mixture was then diluted with saturated aqueous NaHCO₃ and extracted with EtOAc (3×250 mL). The combined organics were washed with brine (250 mL), dried over Na₂SO₄, filtered and concentrated in vacuo. The residue was purified by column chromatography (SiO₂, hexanes/EtOAc=1/0 to 3/1) to give the title compound (20.0 g, 60.2% yield) as a brown solid. LCMS m/z=345.0 [M+H]+

Step 6: Synthesis of 4-bromo-5-chloro-3-fluoro-6-methoxy-2-(tetrahydro-2H-pyran-2-yl)-2H-indazole

Solution 1: the title compound from step 5 (20.0 g, 57.6 mmol) in THF (300 mL). Solution 2: LDA (1 M, 86.4 mL). Solution 3: N-Fluorobenzenesulfonimide (27.2 g, 86.4 mmol) in THF (300 mL). The solution 1 was pumped by Pump 1 {S1, P1, 39.59 mL/min} to flow reactor 1 {FLR1, PFA, Coils reactor, 3.175 (⅛″) mm, 1.021 mL, 20.0° C.}. The solution 2 was pumped by Pump 2 {S2, P2, 11.46 mL/min} to flow reactor 1 {FLR1, PFA, Coils reactor,3.175 (⅛″) mm, 1.021 mL, 0.0° C.}. The solution 3 was pumped by Pump 3 {S3, P3, 39.61 mL/min} to flow reactor 2 {FLR2, PFA, Coils reactor, 3.175 (⅛″) mm, 2.042 mL, 20.0° C.}. The residence time of flow reactor 1 {FLR1, 0.1 min}. The residence time of flow reactor 2 {FLR2, 0.113 min}. Pump 1 and Pump 2 were started at the same time, Pump 3 was started 0.1 min after. The mixture was collected with a bottle. The reaction mixture was quenched by addition water (25 mL) at rt and then diluted with water (250 mL) and extracted with EtOAc (250 mL×3). The combined organic layers were washed with brine (250 mL), dried over Na₂SO₄, filtered and concentrated under reduced pressure. The crude product was purified by column chromatography (SiO₂, hexanes:EtOAc=100/1 to 2/1) to give the title compound (9.0 g, 41.6% yield) as a yellow solid. LCMS m/z=363 [M+H]+

Step 7: Synthesis of tert-butyl (4-(5-chloro-3-fluoro-6-methoxy-2-(tetrahydro-2H-pyran-2-yl)-2H-indazol-4-yl)-3-cyano-7-fluorobenzo[b]thiophen-2-yl)carbamate

A mixture of title compound from step 6 (18.8 g, 51.7 mmol), Intermediate 9 (25.0 g, 62.0 mmol), dichloro(1,1′-bis(diphenylphosphino)ferrocene)palladium(II) (3.78 g, 5.17 mmol) and Cs₂CO₃ (33.6 g, 103 mmol) in dioxane (188 mL) was degassed and purged with N₂ then stirred at 100° C. for 3 h under N₂. The mixture was cooled to rt, diluted with water (400 mL), extracted with EtOAc (200 mL×3). The combined organic layers were washed with brine (200 mL), dried over Na₂SO₄, filtered and concentrated in vacuo. The residue was purified by column chromatography (SiO₂,hexanes:EtOAc=100/1 to 2/1) to give the title compound (12.9 g, 37.9% yield) as a yellow solid. LCMS m/z=575.1 [M+H]+

Step 8: Synthesis of tert-butyl (4-(5-chloro-3-fluoro-6-methoxy-1H-indazol-4-yl)-3-cyano-7-fluorobenzo[b]thiophen-2-yl)carbamate

To a solution of the title compound from step 7 (16.2 g, 28.1 mmol) in DCM (74 mL) was added AcOH (5.08 g, 84.5 mmol, 4.84 mL) and the mixture was heated to 35-40° C. for 3 h. The mixture was cooled to 0-5° C. for 0.5 h and then filtered. The filter cake was washed with DCM and then dried under vacuum to give the title compound (9.1 g, 64.7% yield) as a yellow solid. LCMS m/z=491.1 [M+H]+ 1H NMR (400 MHz, CDCl₃) δ=9.28 (s, 1H), 7.76 (s, 1H), 7.33 (dd, 1H), 7.16 (t, 1H), 6.88 (d, 1H), 4.01 (s, 3H), 1.57 (s, 9H)

### Intermediate 36

### tert-butyl (3-cyano-7-fluoro-4-(3-fluoro-2,7-dihydro-5H-furo[3,4-f]indazol-4-yl)benzo[b]thiophen-2-yl)carbamate

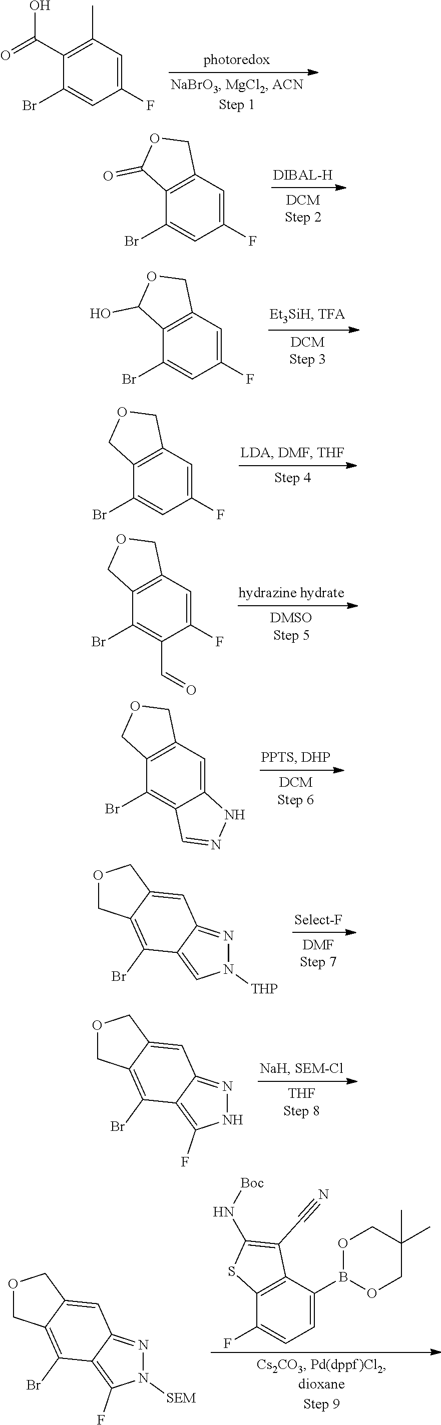

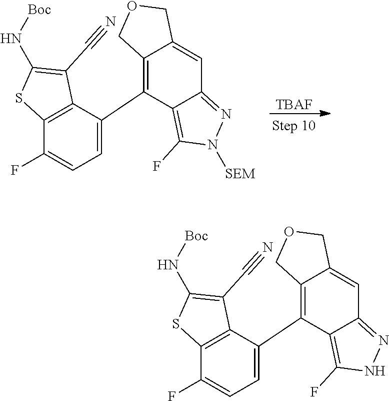

Step 1: Synthesis of 7-bromo-5-fluoroisobenzofuran-1 (3H)-one

A mixture of 2-bromo-4-fluoro-6-methylbenzoic acid (1.00 g, 4.29 mmol), magnesium chloride (409 mg, 4.29 mmol) and sodium bromate (971 mg, 6.44 mmol) in ACN (20 mL) was stirred at 25° C. for 40 h under 420 nm LEDs and N₂. The mixture was then concentrated, diluted with water (50 mL) and extracted with EtOAc (40 mL×3). The combined organic layers were washed with saturated aqueous NaHCO₃ (100 mL×3), dried over Na₂SO₄, filtered and concentrated under reduced pressure to give the title compound (1.5 g, 50% yield) as a white solid. ¹H NMR (400 MHz, 6d-DMSO) δ ppm 7.83-7.78 (m, 1H), 7.63-7.58 (m, 1H), 5.34 (s, 2H).

Step 2: Synthesis of 7-bromo-5-fluoro-1,3-dihydroisobenzofuran-1-ol

To a solution of the title compound from step 1 (2.90 g, 12.6 mmol) in DCM (40 mL) was added DIBAL-H (1 M, 18.8 mL) slowly at −78° C. under N₂, then the mixture was stirred at −78° C. for 1 h. The reaction mixture was quenched by addition 0.5 M aqueous HCl (80 mL) and extracted with DCM (60 mL×3). The combined organic layers were dried over Na₂SO₄, filtered and concentrated under reduced pressure to give the title compound (2.76 g, 94% yield) a yellow oil.

Step 3: Synthesis of 4-bromo-6-fluoro-1,3-dihydroisobenzofuran

To a solution of the title compound from step 2 (2.76 g, 11.8 mmol) in DCM (30 mL) was added Et₃SiH (8.26 g, 71.1 mmol, 11.6 mL) slowly at 0° C., after 0.17 h, TFA (4.05 g, 35.5 mmol, 2.64 mL) was added to the mixture slowly at 0° C., the mixture was warmed to 25° C. and stirred 4 h. The mixture was quenched by addition of saturated aq. NaHCO₃ (80 mL) and extracted with DCM (60 mL×3). The combined organic layers were dried over Na₂SO₄, filtered and concentrated under reduced pressure. The residue was purified by silica gel column chromatography (PE:EtOAc=1:0 to 5:1) to give the title compound (2.50 g, 97% yield) as a white solid. ¹H NMR (400 MHz, CDCl₃) δ ppm 7.18-7.12 (m, 1H), 6.89 (d, 1H), 5.18 (s, 2H), 5.05 (d, 2H).

Step 4: Synthesis of 4-bromo-6-fluoro-1,3-dihydroisobenzofuran-5-carbaldehyde

To a solution of the title compound from step 3 (2.30 g, 10.6 mmol) in THF (46 mL) was added LDA (2 M, 8 mL) at −78° C. under N₂. After 10 minutes, DMF (3.87 g, 53.0 mmol, 4 mL) in THF (5 mL) was added to the mixture at −78° C., then the mixture was warmed to 0° C. and stirred for 30 min. The mixture was quenched by addition saturated aqueous NH₄Cl (20 mL), diluted with water (30 mL) and extracted with EtOAc (50 mL×3). The combined organic layers were washed with brine (50 mL×3), dried over Na₂SO₄, filtered and concentrated under reduced pressure. The residue was purified by silica gel column chromatography (PE:EtOAc=1:0 to 5:1) to afford the title compound (1.62 g, 62% yield) as a yellow solid. ¹H NMR (400 MHz, CDCl₃) δ ppm 10.35 (s, 1H), 7.03 (d, 1H), 5.24 (s, 2H), 5.12 (s, 2H).

Step 5: Synthesis of 4-bromo-5,7-dihydro-1H-furo[3,4-f]indazole

To a solution of the title compound in Step 4 (1.62 g, 6.61 mmol) in DMSO (32.4 mL) was added hydrazine hydrate (3.34 g, 56.8 mmol, 3.24 mL), the mixture was heated to 120° C. and stirred for 16 h. The reaction mixture was diluted with EtOAc (80 mL) and washed with brine (100 mL×3), dried over NaSO₄, filtered and concentrated under reduced pressure to give the title compound (1.49 g, 61% yield) as a white solid. ¹H NMR (400 MHz, CDCl₃) δ ppm 10.43-10.14 (m, 1H), 8.07 (s, 1H), 7.25 (s, 1H), 5.27 (s, 2H), 5.15 (s, 2H).

Step 6: Synthesis of 4-bromo-2-(tetrahydro-2H-pyran-2-yl)-2,7-dihydro-5H-furo[3,4-f]indazole

To a solution of the title compound (1.49 g, 6.23 mmol) in DCM (30 mL) was added DHP (1.57 g, 18.70 mmol, 1.7 mL) and PPTS (157 mg, 623 μmol), the mixture was stirred at 35° C. for 1 h. The mixture was quenched by addition of saturated aqueous NaHCO₃ (40 mL) and extracted with DCM (30 mL×3). The combined organic layers were dried over Na₂SO₄, filtered and concentrated under reduced pressure. The residue was purified by silica gel column chromatography (PE:EtOAc=1:0 to 3:1) to afford the title compound (1.46 g, 72% yield) as an off-white solid. ¹H NMR (400 MHz, CDCl₃) δ ppm 8.15 (s, 1H), 7.44 (s, 1H), 5.69-5.64 (m, 1H), 5.21 (s, 2H), 5.08 (s, 2H), 4.18-4.13 (m, 1H), 3.84-3.76 (m, 1H), 2.25-2.18 (m, 2H), 2.12-2.04 (m, 1H), 1.81-1.75 (m, 2H), 1.72-1.67 (m, 1H).

Step 7: Synthesis of 4-bromo-3-fluoro-2,7-dihydro-5H-furo[3,4-f]indazole

To a solution of the title compound from step 6 (0.88 g, 2.72 mmol) in DMF (8.8 mL) was added Select-F (3.86 g, 10.89 mmol), the mixture was stirred at 45° C. for 1 h. The mixture was diluted with EtOAc (50 mL), washed with brine (50 mL×2) and the organic layer was dried over Na₂SO₄, filtered and concentrated to give a residue. The residue was purified by silica gel column chromatography (PE:EtOAc=1:0 to 3:1) to afford the title compound (830 mg, 47% yield) as a white solid. ¹H NMR (400 MHz, 6d-DMSO) δ ppm 13.05-12.60 (m, 1H), 7.40 (s, 1H), 5.15 (s, 2H), 4.99 (s, 2H).

Step 8: Synthesis of 4-bromo-3-fluoro-2-((2-(trimethylsilyl)ethoxy)methyl)-2,7-dihydro-5H-furo[3,4-f]indazole

To a solution of the title compound step 7 (970 mg, 3.77 mmol) in THF (20 mL) was added NaH (166 mg, 4.15 mmol, 60% in mineral oil) under N₂ at 25° C., after 15 minutes, SEM-Cl (692 mg, 4.15 mmol, 735 μL) was added and the mixture was stirred at 25° C. for 15 min. The mixture was quenched by addition saturated aqueous NH₄Cl (30 mL) and extracted with EtOAc (20 mL×3). The combined organic layers were dried over Na₂SO₄, filtered and concentrated under reduced pressure to give a residue. The residue was purified by silica gel column chromatography (PE:EtOAc=1:0 to 5:1) to afford the title compound. (1.00 g, 68% yield) as a white solid. ¹H NMR (400 MHz, CDCl₃) δ ppm 7.25 (s, 1H), 5.55 (s, 1H), 5.26 (s, 2H), 5.13 (s, 2H), 3.58-3.53 (m, 2H), 0.91-0.87 (m, 2H), 0.03 (s, 9H).

Step 9: Synthesis of tert-butyl (3-cyano-7-fluoro-4-(3-fluoro-2-((2-(trimethylsilyl)ethoxy)methyl)-2,7-dihydro-5H-furo[3,4-f]indazol-4-yl)benzo[b]thiophen-2-yl)carbamate

A mixture of the title compound from step 8 (500 mg, 1.29 mmol), Intermediate 9 (574 mg, 1.42 mmol), Cs₂CO₃ (841 mg, 2.58 mmol) and Pd(dppf)Cl₂ (94.0 mg, 129 μmol) in dioxane (10 mL) was degassed and purged with nitrogen, and then the mixture was stirred at 100° C. for 5 h under N₂. The mixture was filtered and concentrated under reduced pressure. The residue was purified by silica gel column chromatography (PE:EtOAc=1:0 to 3:1) to afford the title compound (600 mg, 78% yield) as a yellow oil. ¹H NMR (400 MHz, CDCl₃) δ ppm 7.76 (s, 1H), 7.42 (s, 1H), 7.37-7.33 (m, 1H), 7.16-7.10 (m, 1H), 5.70-5.64 (m, 1H), 5.58-5.53 (m, 1H), 5.33-5.23 (m, 2H), 4.96-4.81 (m, 2H), 3.62-3.54 (m, 2H), 1.58 (s, 9H), 0.96-0.90 (m, 2H), 0.02 (s, 9H).

Step 10: Synthesis of tert-butyl (3-cyano-7-fluoro-4-(3-fluoro-2,7-dihydro-5H-furo[3,4-f]indazol-4-yl)benzo[b]thiophen-2-yl)carbamate

A solution of title compound from step 9 (550 mg, 919 μmol) in TBAF (1 M, 5.5 mL) in flow reactor, mixture was heated to 135° C. and stirred for 15 min, then diluted with water (50 mL) and extracted with EtOAc (40 mL×3). The combined organic layers were washed with brine (50 mL×3), dried over Na₂SO₄, filtered and concentrated under reduced pressure. The residue was purified by silica gel column chromatography (PE:EtOAc=1:0 to 1:1) to afford the title compound (280 mg, 58% yield) as an off-white solid. ¹H NMR (400 MHz, CDCl₃) δ ppm 9.29 (s, 1H), 7.37-7.32 (m, 1H), 7.29 (s, 1H), 7.16-7.01 (m, 1H), 5.33-5.20 (m, 2H), 4.96-4.80 (m, 2H), 1.57 (s, 9H).

### Intermediate 37

### methyl 4-bromo-5-chloro-3-fluoro-1H-indazole-6-carboxylate

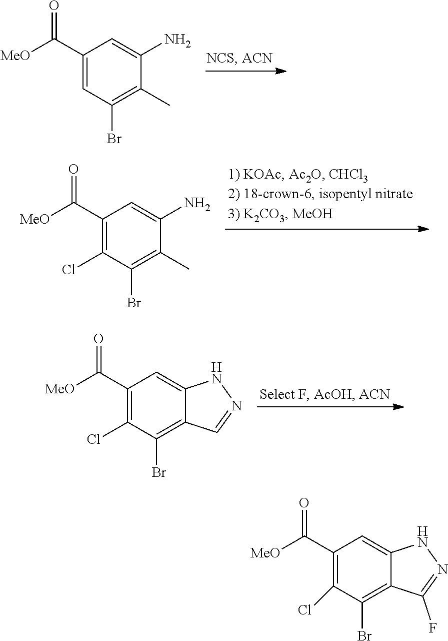 [SMILES: COC(=O)c1cc(N)c(C)c(Br)c1 ; COC(=O)c1cc(N)c(C)c(Br)c1Cl ; COC(=O)c1cc2[nH]ncc2c(Br)c1Cl ; COC(=O)c1cc2[nH]nc(F)c2c(Br)c1Cl]

Step 1: Synthesis of methyl 5-amino-3-bromo-2-chloro-4-methylbenzoate

To a solution of methyl 3-amino-5-bromo-4-methylbenzoate (250 g, 942 mmol) in ACN (2.5 L) was added NCS (125 g, 942 mmol) at 15-25° C., then the reaction mixture was stirred at 80° C. for 2 h then concentrated in vacuo. The residue was purified by column chromatography (SiO₂, hexanes:EtOAc=20/1 to 3/1) to give the title compound (206 g, 31% yield) as a brown solid. LCMS m/z=277.8 [M+H]+

Step 2: Synthesis of methyl 4-bromo-5-chloro-1H-indazole-6-carboxylate

A mixture of the title compound from step 1 (103 g, 369 mmol), potassium acetate (43.5 g, 443 mmol), acetic anhydride (151 g, 1.48 mol, 138 mL) in chloroform (960 mL) was stirred at 60-65° C. for 1 h, then 18-crown-6 (9.77 g, 36.9 mmol) and isopentyl nitrite (433 g, 3.7 mol, 497 mL) was added, then the mixture was stirred at 65° C. for 3 h. K₂CO₃ (153 g, 1.1 mol) was added portionwise and MeOH (500 mL) was added and stirred at 65° C. for 1 h. The mixture was diluted with water (1 L), extracted with EtOAc (1 L×3). The combined organic layers were washed with brine (1 L), dried over Na₂SO₄, filtered and concentrated in vacuo. The crude product was triturated with hexanes:EtOAc=10:1 (500 mL) at 25° C. for 0.5 h to give the title compound (160 g, 73% yield) as a brown solid. LCMS m/z=288.8 [M+H]+

Step 3: Synthesis of methyl 4-bromo-5-chloro-3-fluoro-1H-indazole-6-carboxylate

To a solution of the title compound from step 2 (150 g, 518 mmol) in ACN (1.3 L) was added AcOH (93.3 g, 1.5 mol, 88.9 mL) and Select-F (220 g, 621 mmol), the mixture was stirred at 80° C. for 16 h. The mixture was cooled to rt, filtered washing with ACN. The filtrate was concentrated in vacuo diluted with EtOAc (1 L), washed with brine (2×500 mL) and the organic layer was dried over Na₂SO₄, filtered and concentrated to give a residue. The crude product was purified by reversed-phase HPLC (0.2% FA condition) to afford the title compound (60 g, 36.6% yield) as a brown solid. LCMS m/z=370.0 [M+H]+

### Intermediate 38

### tert-butyl (4-(5-chloro-3-fluoro-6-(hydroxymethyl)-1H-indazol-4-yl)-3-cyano-7-fluorobenzo[b]thiophen-2-yl)carbamate

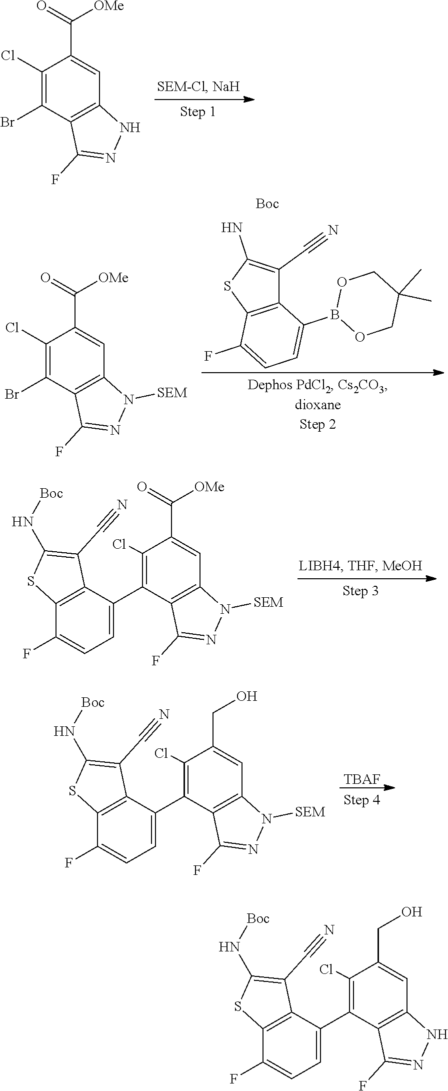

Step 1: Synthesis of methyl 4-bromo-5-chloro-3-fluoro-1-(2-trimethylsilylethoxymethyl)indazole-6-carboxylate

To a solution of Intermediate 37 (10 g, 32.52 mmol) in THF (200 mL) was added NaH (1.43 g, 35.77 mmol, 60% purity, 1.1 eq) at 0° C. under N₂, after 15 min, SEM-Cl (5.96 g, 35.77 mmol, 6.33 mL, 1.1 eq) was added to the mixture at 0° C., then the mixture was warmed to 25° C. and stirred for 45 min. The reaction mixture was quenched by saturated aqueous NH₄Cl solution (100 mL) and extracted with EtOAc (100 mL×3). The combined organic layers were dried over Na₂SO₄, filtered and concentrated under reduced pressure to afford the title compound (14 g, crude) as brown oil. ¹H NMR (CDCl₃, 400 MHz) δ 7.8-7.9 (m, 1H), 5.58 (s, 2H), 3.99 (s, 3H), 3.57 (d, 2H), 0.89 (s, 2H), −0.03 (s, 9H)

Step 2: Synthesis of methyl 4-[2-(tert-butoxycarbonylamino)-3-cyano-7-fluoro-benzothiophen-4-yl]-5-chloro-3-fluoro-1-(2-trimethylsilylethoxymethyl)indazole-6-carboxylate

A mixture of the title compound from step 1 (7 g, 15.9 mmol), Intermediate 9 (9.70 g, 23.9 mmol), [2-(2-diphenylphosphanylphenoxy)phenyl]-diphenyl-phosphane; palladium(II); dichloride (2.29 g, 3.20 mmol), Cs₂CO₃ (13.0 g, 39.9 mmol) in dioxane (100 mL) was degassed and purged with N₂ then stirred at 100° C. for 12 h under N₂. The reaction mixture was then diluted with water (100 mL) and extracted with EtOAc (100 mL×3). The combined organic layers were dried over Na₂SO₄, filtered and concentrated under reduced pressure. The residue was purified by column chromatography (SiO₂, hexanes:EtOAc=10/1 to 1/1) to give the title compound (3.2 g, 4.93 mmol, 30% yield) as a yellow oil. 1H NMR (CDCl₃, 400 MHz) δ 8.05 (d, 1H), 7.7-7.8 (m, 1H), 7.34 (dd, 1H), 7.1-7.2 (m, 1H), 5.6-5.7 (m, 2H), 4.01 (s, 3H), 3.59 (dd, 2H), 1.56 (s, 9H), 0.94 (td, 2H), 0.0-0.0 (m, 9H)

Step 3. Synthesis of tert-butyl N-[4-[5-chloro-3-fluoro-6-(hydroxymethyl)-1-(2-trimethylsilylethoxymethyl) indazol-4-yl]-3-cyano-7-fluoro-benzothiophen-2-yl]carbamate
Solution 1: the title compound from step 2 (4.6 g, 7.09 mmol) was dissolved in THF (62 mL) and MeOH (10 mL);
Solution 2: lithium borohydride (2 M, 12.40 mL)

The solution 1 was pumped by Pump 1 {S1, P1, 10.5 mL/min} to flow reactor 1 {FLR1, SS, Static mixer, 4.76 ( 3/16″) mm, 0.93 mL, 25° C.} {FLR1, PFA, Coils reactor, 3.175 (⅛″) mm, 21 mL, 25° C.}. The solution 2 was pumped by Pump 2 {S2, P2, 2.4 mL/min} to flow reactor 1 {FLR1, SS, Static mixer, 4.76 ( 3/16″) mm, 0.93 mL, 25° C.} {FLR 1, PFA, Coils reactor, 3.175 (⅛″) mm, 31 mL, 25° C.}. The residence time of flow reactor 1 was {FLR1, 5 min}. Pump 2 was started after 2 min Pump 1 was started. The reaction mixture was quenched by saturated NH₄Cl 50 mL at 0° C., stirred at 25° C. for 10 min, and then extracted with EtOAc (60 mL×3). The combined organic layers were washed with brine (30 mL), dried over Na₂SO₄, filtered and concentrated under reduced pressure. The residue was purified by column chromatography (SiO₂, hexanes:EtOAc=3/1 to 1/1) to give the title compound (4.3 g, crude) as yellow solid. ¹H NMR (400 MHz, DMSO-d₆) δ 11.5-11.7 (m, 1H), 7.96 (s, 1H), 7.3-7.5 (m, 2H), 5.80 (br t, 1H), 5.6-5.7 (m, 2H), 4.71 (br d, 2H), 3.54 (s, 2H), 1.50 (s, 9H), 0.83 (t, 2H), −0.06 (s, 9H)

Step 4: Synthesis of tert-butyl N-[4-[5-chloro-3-fluoro-6-(hydroxymethyl)-1H-indazol-4-yl]-3-cyano-7-fluoro-benzothiophen-2-yl]carbamate.

A solution of the title compound from step 3 (3.2 g, 5.15 mmol) in TBAF (1 M, 32 mL) was stirred at 135° C. for 20 min. The reaction mixture was quenched by water (80 mL) at 0° C., then extracted with EtOAc (60 mL×3). The combined organic layers were washed with brine 30 mL, dried over Na₂SO₄, filtered and concentrated under reduced pressure to give a residue. The residue was purified by column chromatography (SiO₂, hexanes:EtOAc=1/1 to 2/1) to give the title compound (1.4 g, 55% yield) as a yellow solid. ¹H NMR (DMSO-d₆, 400 MHz) δ 12.74 (s, 1H), 7.68 (s, 1H), 7.2-7.4 (m, 2H), 5.73 (t, 1H), 4.5-4.8 (m, 2H), 1.47 (s, 9H)

### Intermediate 39

### 2-(4-bromo-5-chloro-3-fluoro-6-methyl-1H-indazol-1-yl)-4-((S)-1-((S)-1-methylpyrrolidin-2-yl)ethoxy)-7,8-dihydro-5H-pyrano[4,3-d]pyrimidine

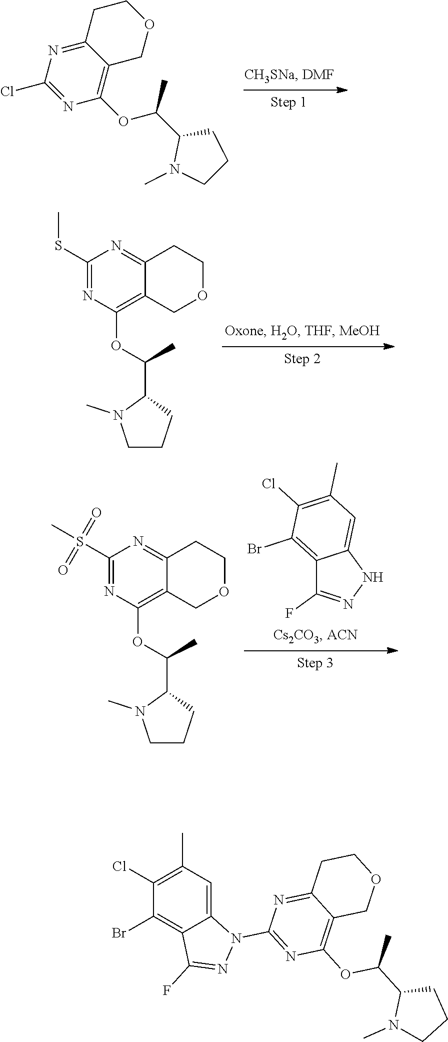

Step 1: Synthesis of 4-((S)-1-((S)-1-methylpyrrolidin-2-yl)ethoxy)-2-(methylthio)-7,8-dihydro-5H-pyrano[4,3-d]pyrimidine

To a solution of Intermediate 1 (35.0 g, 117 mmol) in DMF (350 mL) was added CH₃SNa (19.4 g, 276 mmol, 17.6 mL) into rt. The mixture was heated to 50° C. and stirred for 12 h. Upon cooling to rt the mixture was poured into H₂O (1.0 L) and extracted with EtOAc (3×1.0 L). The combined organic phase was washed with brine (2×1.0 L), dried and concentrated under vacuum to give the title compound (30.0 g, 93.1 mmol, 79.2% yield) as a yellow oil. LCMS m/z=310.2 [M+H]+

Step 2: Synthesis of 4-((S)-1-((S)-1-methylpyrrolidin-2-yl)ethoxy)-2-(methylsulfonyl)-7,8-dihydro-5H-pyrano[4,3-d]pyrimidine

To a solution of oxone (57.6 g, 93.7 mmol) in H₂O (290 mL) cooled to 0° C. was slowly added a solution of the title compound from step 1 (14.5 g, 46.9 mmol) in MeOH (116 mL) and THF (116 mL) and the mixture was then allowed to warm to rt and stirred for 4 h. The mixture was filtered and diluted with EtOAC (200 mL), quenched with Na₂SO₃ (34.0 g, aqueous solution) at 0-10° C. and pH was adjusted (pH=8) with Na₂CO₃ (solid) at 0-10° C. The layers were separated and aqueous was extracted with EtOAc (3×200 mL). The combined organic phase was washed with brine (200 mL), dried and concentrated under vacuum to give the title compound (11.0 g, 30.7 mmol, 33.2% yield) as a yellow oil. LCMS m/z=342.1 [M+H]+

Step 3: Synthesis of 2-(4-bromo-5-chloro-3-fluoro-6-methyl-1H-indazol-1-yl)-4-((S)-1-((S)-1-methylpyrrolidin-2-yl)ethoxy)-7,8-dihydro-5H-pyrano[4,3-d]pyrimidine

Three reactions were performed in parallel as follows. To a solution of the title compound from Step 2 (4.00 g, 11.7 mmol) in ACN (40.0 mL) at rt was added the title compound from Example 27 & 28, Step 1 (2.99 g, 11.4 mmol) and Cs₂CO₃ (7.63 g, 23.43 mmol). The mixture was heated 65° C. and stirred for 1 h. The 3 reactions were cooled to rt, combined and poured into water (500 mL) and extracted to DCM (2×200 mL). The combined organic phase was dried over Na₂SO₄ and concentrated under vacuum. The crude product was triturated with hexanes:EtOAc=1:1 (5 V) at 20-25° C. for 1 h to give the title compound (11.0 g, 20.1 mmol, 55.5% yield) as a white solid. LCMS m/z=526.1 [M+H]⁺. ¹H NMR 400 MHz, CDCl₃ δ 8.56 (s, 1H), 5.45-5.38 (m, 1H), 4.76-4.59 (m, 2H), 4.06 (dt, J=2.0, 6.0 Hz, 2H), 3.11 (s, 1H), 3.01 (t, J=5.6 Hz, 2H), 2.70-2.63 (m, 1H), 2.60 (s, 3H), 2.48 (s, 3H), 2.38-2.29 (m, 1H), 2.02-1.91 (m, 1H), 1.85-1.72 (m, 3H), 1.44 (d, J=6.0 Hz, 3H)

### Intermediate 40

### tert-butyl (4-(5-chloro-3-fluoro-1H-pyrazolo[3,4-c]pyridin-4-yl)-3-cyano-7-fluorobenzo[b]thiophen-2-yl)carbamate

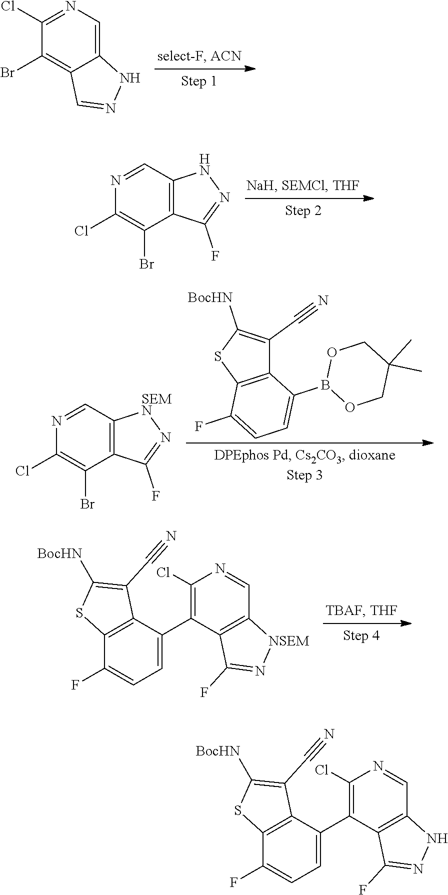

Step 1: Synthesis of 4-bromo-5-chloro-3-fluoro-1H-pyrazolo[3,4-c]pyridine

A mixture of 4-bromo-5-chloro-1H-pyrazolo[3,4-c]pyridine (600 mg, 2.58 mmol) and select-F (1.37 g, 3.87 mmol) in ACN (15 mL) was stirred at 80° C. for 48 h.

The reaction was filtered, evaporated and purified by Prep-TLC with PE:EA=1:1 to afford the title compound (200 mg, 30.9%) as a yellow solid. LCMS m/z=252 [M+H]+

Step 2: Synthesis of 4-bromo-5-chloro-3-fluoro-1-((2-(trimethylsilyl)ethoxy)methyl)-1H-pyrazolo[3,4-c]pyridine:

NaH (190 mg, 4.78 mmol, 60% in mineral oil) was added to a solution of the title compound from step 1 (600 mg, 2.39 mmol) in THF (10 mL) at 0° C. and the resulting mixture was stirred at rt for 30 min. Then SEM-C1 (596 mg, 3.58 mmol) was added at 0° C. and the resulting mixture was stirred at rt for 2 h. The reaction was quenched with aqueous NH₄Cl (20 mL) and extracted with EtOAc (20 mL×3). The organic layer was washed with brine (30 mL), dried over Na₂SO₄ and evaporated to afford crude product. The crude product was purified by Prep-TLC with PE:EA=3:1 to afford the title compound (300 mg, 33.0%) as a yellow oil. LCMS m/z=382 [M+H]+

Step 3: Synthesis of tert-butyl (4-(5-chloro-3-fluoro-1-((2-(trimethylsilyl)ethoxy)methyl)-1H-pyrazolo[3,4-c]pyridin-4-yl)-3-cyano-7-fluorobenzo[b]thiophen-2-yl)carbamate

A mixture of the title compound from step 2 (300 mg, 787 μmol), Intermediate 9 (477 mg, 1.18 mmol), Cs₂CO₃ (511 mg, 1.57 mmol) and DPEphos PdCl₂ (56.3 mg, 78.7 μmol) in dioxane (5 mL) was stirred at 100° C. for 3 h under N₂. The reaction solution was poured into water (10 mL) and extracted with EtOAc (10 mL×3). The organic layer was washed with brine (10 mL), dried over Na₂SO₄ and evaporated. The crude product was purified by Prep-TLC with PE:EA=3:1 to afford the title compound (150 mg, 32.1%) as a yellow solid. LCMS m/z=592 [M+H]+

Step 4: Synthesis of tert-butyl (4-(5-chloro-3-fluoro-1H-pyrazolo[3,4-c]pyridin-4-yl)-3-cyano-7-fluorobenzo[b]thiophen-2-yl)carbamate

A mixture of the title compound from step 3 (140 mg, 236 μmol) in 1 M TBAF in THF (3 mL) was stirred at 60° C. for 16 h. The reaction solution was poured into aqueous NH₄Cl (3 mL) and extracted with EtOAc (3 mL×3). The organic layer was washed with brine (5 mL), dried over Na₂SO₄ and evaporated. The crude product was purified by Prep-TLC with DCM:MeOH=15:1 to afford the title compound (90 mg, 82.5%) as a yellow solid. LCMS m/z=462 [M+H]+

### Intermediate 41

### tert-butyl (4-(5-chloro-6-methoxy-1H-benzo[d][1,2,3]triazol-4-yl)-3-cyano-7-fluorobenzo[b]thiophen-2-yl)carbamate

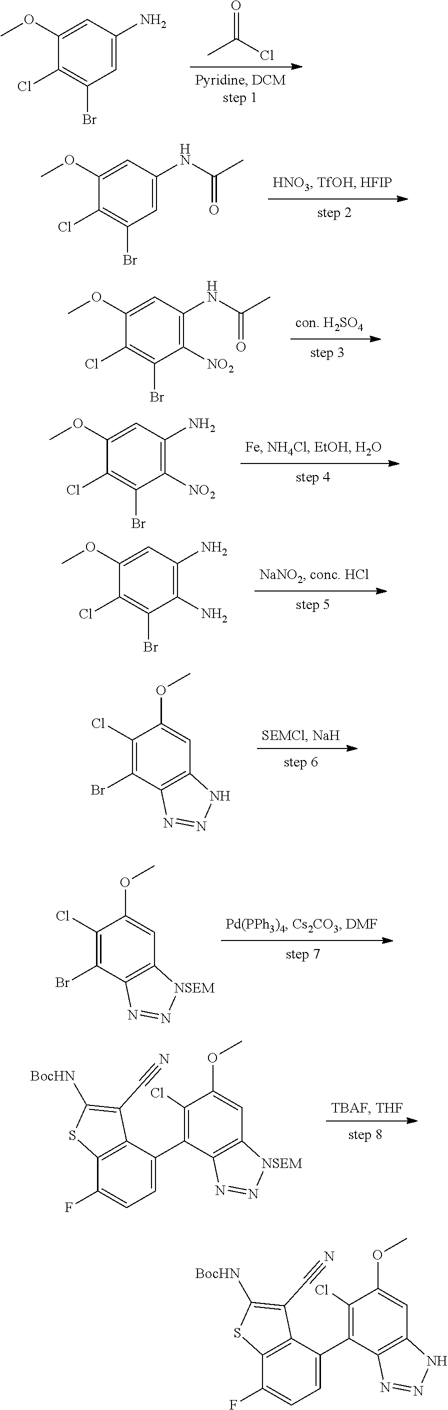

Step 1: Synthesis of N-(3-bromo-4-chloro-5-methoxyphenyl) acetamide

To a solution of 3-bromo-4-chloro-5-methoxyaniline (25 g, 105.71 mmol) in DCM (50 ml) was added pyridine (12.54 g, 158.57 mmol) at room temperature. Then acetyl chloride (8.3 g, 105.71 mmol) was added to the mixture at 0° C. The resulting mixture was stirred at room temperature for 2 h. The mixture was poured into water (200 mL) and extracted with DCM (3×200 mL). The organic layer was washed with brine (100 mL), dried with Na₂SO₄ and concentrated under vacuum. The crude product was purified by flash with DCM:MeOH=20:1 to afford the title compound (21 g, 71.3%) as a yellow solid. LCMS m/z=278 [M+H]+

Step 2: Synthesis of N-(3-bromo-4-chloro-5-methoxy-2-nitrophenyl) acetamide

TfOH (5.66 g, 37.7 mmol) was added to a mixture of the title compound from step 1 (21 g, 75.4 mmol) and 68% HNO₃ (5.46 m1, 82.93 mmol) in HFIP (50 mL) and stirred at 80° C. for 2 h. The mixture was added to saturated aqueous NaHCO₃ (150 mL) and extracted with DCM (3×150 mL). The organic layer was dried with Na₂SO₄ and concentrated under vacuum. The crude product was purified by flash silica chromatography with DCM:MeOH=20:1 to afford the title compound (18 g, 76%) as a yellow solid. LCMS m/z=321 [M−H]

Step 3: Synthesis of 3-bromo-4-chloro-5-methoxy-2-nitroaniline

The title compound from step 2 (18 g, 55.6 mmol) in conc. H₂SO₄ (50 mL) was stirred at 60° C. for 2 h. Ice water was added to the mixture and the resultant precipitate was collected via filtration, dried via vacuum pump to afford the title compound (14 g, 89.3%) as a yellow solid. LCMS m/z=279 [M−H]

Step 4: Synthesis of 3-bromo-4-chloro-5-methoxybenzene-1,2-diamine

A mixture of the title compound from step 3 (14 g, 49.7 mmol), Fe (13.9 g, 247.8 mmol) and NH₄Cl (13.2 g, 247.8 mmol) in EtOH (50 mL): H₂O (10 mL) was stirred at 80° C. for 2 h. The mixture was filtered and the filtrate was poured into water (150 mL) and extracted with EtOAc (3×150 mL). The organic layer was washed with brine (50 mL), dried over Na₂SO₄ and evaporated to afford the title compound (11 g, crude) as a yellow solid, which was used directly for next step without further purification. LCMS m/z=251 [M+H]

Step 5: Synthesis of 4-bromo-5-chloro-6-methoxy-1H-benzotriazole

To a stirred solution of the title compound from step 4 (11 g, 43.67 mmol) in conc. HCl (352 mL, 349.36 mmol) was added NaNO₂ (23.98 g, 349.36 mmol) dropwise in water (10 mL) at room temperature under air atmosphere. The resulting mixture was stirred for 10 min then treated with ice water. The resultant precipitate was collected via filtration and dried to afford the title compound (8 g, 69.7%) as a yellow solid. LCMS m/z=262 [M+H]

Step 6: Synthesis of 4-bromo-5-chloro-6-methoxy-1-{[2-(trimethylsilyl)ethoxy]methyl}-1H-benzotriazole

To a solution of the title compound from step 5 (8 g, 30.4 mmol) in THF (50 mL) was added NaH (2.184 g, 91.2 mmol, 60% in oil) at 0° C. The resulting mixture was stirred at rt for 30 min. Then SEM-C1 (7.6 g, 45.6 mmol) was added to the mixture at 0° C. The resulting mixture was stirred at rt for 2 h. The reaction was quenched with aq. NH₄Cl (20 mL). Then the mixture was extracted with EtOAc (3×100 mL) and H₂O (100 mL). The organic layer was dried with Na₂SO₄ and concentrated under vacuum to afford crude product. The crude product was purified by flash chromatography with PE:EA=15:1 to afford the title compound (7 g, 58.4%) as a yellow solid. LCMS m/z=392 [M+H]

Step 7: Synthesis of tert-butyl (4-(5-chloro-6-methoxy-1-((2-(trimethylsilyl)ethoxy)methyl)-1H-benzo[d][1,2,3]triazol-4-yl)-3-cyano-7-fluorobenzo[b]thiophen-2-yl)carbamate

A mixture of the title compound from step 6 (7 g, 27.83 mmol), Intermediate 9 (11.2 g, 27.83 mmol), Pd(PPh₃)₄ (6.43 g, 5.56 mmol) and Cs₂CO₃ (18.15 g, 55.66 mmol) in DMF (50 ml) was stirred at 100° C. for 2 h. The reaction was poured into water (100 mL) and extracted with EtOAc (3×100 mL). The organic layer was washed with brine (50 mL), dried over Na₂SO₄ and concentrated under vacuum. The crude product was purified by flash silica chromatography with PE:EA=4:1 to afford the title compound (3.2 g, 43.8%) as a white solid. LCMS m/z=604 [M+H]

Step 8: Synthesis of tert-butyl [4-(5-chloro-6-methoxy-1H-benzotriazol-4-yl)-3-cyano-7-fluoro-1-benzothiophen-2-yl]carbamate:

A mixture of the title compound from step 7 (3.2 g, 5.3 mmol) in TBAF (20 ml, 1 M THF solution) was stirred at 60° C. for 12 h. The mixture was poured into aq. NH₄Cl (100 mL) and extracted with EtOAc (3×100 mL). The organic layer was washed with brine (50 mL), dried over Na₂SO₄ and concentrated. The crude product was purified by Prep-TLC with DCM: MeOH=20:1 to afford the title compound (1.8 g, 71.7%) as a yellow solid. LCMS m/z=474 [M+H]

### Intermediate 42

### tert-butyl (3-cyano-4-(5-(difluoromethyl)-3-fluoro-6-methyl-1H-indazol-4-yl)-7-fluorobenzo[b]thiophen-2-yl)carbamate

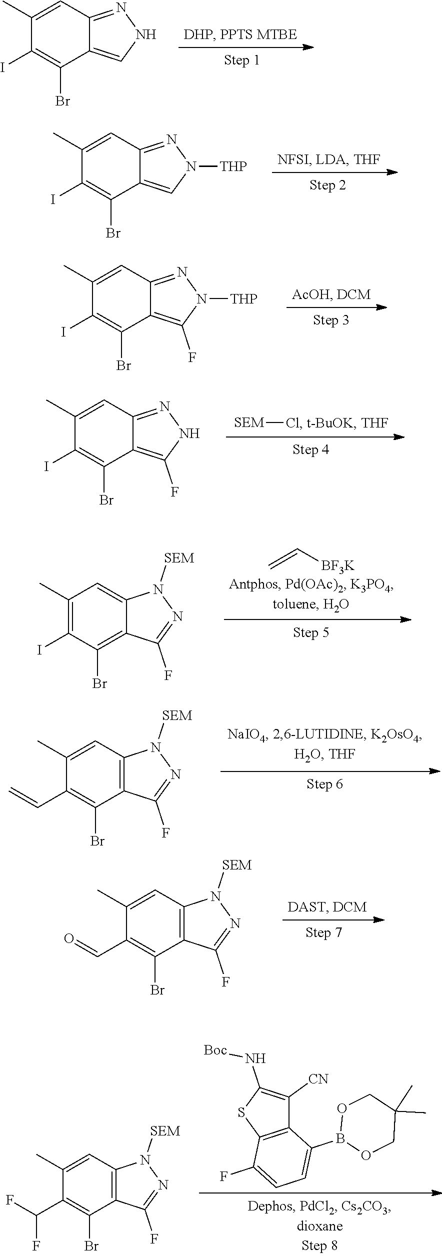

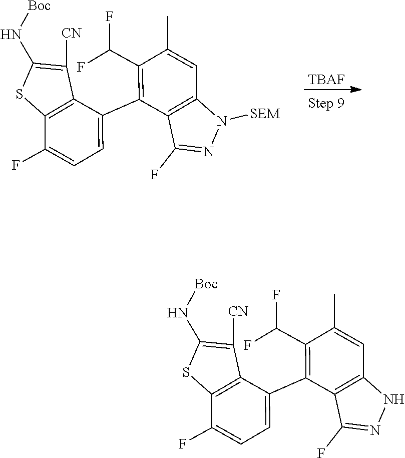 [SMILES: Cc1cc2c(c(F)nn2COCC[Si](C)(C)C)c(-c2ccc(F)c3sc(NC(=O)OC(C)(C)C)c(C#N)c23)c1C(F)F ; "TBAF" ; Cc1cc2[nH]nc(F)c2c(-c2ccc(F)c3sc(NC(=O)OC(C)(C)C)c(C#N)c23)c1C(F)F]

Step 1: Synthesis of 4-bromo-5-iodo-6-methyl-2-(tetrahydro-2H-pyran-2-yl)-2H-indazole

To a solution of 4-bromo-5-iodo-6-methyl-1H-indazole (100 g, 296 mmol) in MBTE (500 mL) was added PPTS (7.46 g, 29.6 mmol) and DHP (49.9 g, 594 mmol). The mixture was stirred at 40° C. for 16 h. The reaction mixture was quenched by the addition of H₂O (1.00 L) at 25° C. and then extracted with MTBE (2×500 mL). The combined organic layers were washed with brine (500 mL) and concentrated under reduced pressure. The crude product was purified by re-crystallization from acetone (300 mL) at 50° C. The filtrate was concentrated under reduced pressure, and the residue was purified by column chromatography (SiO₂, hexanes:EtOAc=100/1 to 5/1) to afford the title compound (83.0 g, 197 mmol, 66.4% yield) as a white solid. H NMR (400 MHz, DMSO-d₆) δ 8.41 (s, 1H), 7.64 (s, 1H), 5.74 (dd, 1H), 3.92-4.07 (m, 1H), 3.65-3.78 (m, 1H), 2.58 (s, 3H), 2.13-2.25 (m, 1H), 1.99-2.10 (m, 1H), 1.87-1.99 (m, 1H), 1.64-1.78 (m, 1H), 1.55-1.63 (m, 2H).

Step 2: Synthesis of 4-bromo-3-fluoro-5-iodo-6-methyl-2-(tetrahydro-2H-pyran-2-yl)-2H-indazole
Solution 1: The title compound from step 1 (53.0 g, 126 mmol)) in THF (800 mL).
Solution 2: {LDA (1.00 M, 176 mL)} at 20-25° C.
Solution 3: {NFSI (59.5 g, 188 mmol)) in THF (800 mL).

Solution 1 was pumped by Pump 1 {S1, P1, 41.45 mL/min} to flow reactor 1 {FLR1, PFA, Coils reactor, 3.175 (⅛″) mm, 5.0 mL, 0° C.}. Solution 2 was pumped by Pump 2 {S2, P2, 8.55 mL/min} to flow reactor 1 {FLR1, PFA, Coils reactor, 3.175 (⅛″) mm, 5.0 mL, 0° C.}. Solution 3 was pumped by Pump 3 {S3, P3, 41.806 mL/min} to flow reactor 2 {FLR2, PFA, Coils reactor, 3.175 (⅛″) mm, 10.0 mL, 0° C.}. The residence time of flow reactor 1 {FLR1, 0.1 min}. The residence time of flow reactor 2 {FLR2, 0.109 min}. The mixture was collected with a bottle and quenched by 500 mL H₂O at 20° C., diluted with H₂O (500 mL) and extracted with EtOAc (2×1.0 L). The combined organic layers were washed with brine (2×1.0 L) and saturated aqueous NaHCO₃ (1.0 L), dried over anhydrous Na₂SO₄, filtered and concentrated under reduced pressure. The residue was purified by column chromatography (SiO₂, hexanes:EtOAc=100/1 to 100/6) to afford the title compound (22.0 g, 39.0 mmol, 31.0% yield, 77.8% purity) was obtained as a white solid. The material was carried forward without further purification.

Step 3: Synthesis of 4-bromo-3-fluoro-5-iodo-6-methyl-2H-indazole

A mixture of the title compound from step 2 (22.0 g, 50.1 mmol) and AcOH (4.51 g, 75.2 mmol) in DCM (132 mL) was degassed and purged with N₂ (3 times) then the mixture was stirred at 40° C. for 16 h under N₂. The mixture was cooled to 0° C. and the resultant solid was collected via filtration, washing with three 50 mL portions of cold water and then dried at 50° C. to afford the title compound (12.4 g, 34.8 mmol, 69.4% yield) as a white solid.

Step 4: Synthesis of 4-bromo-3-fluoro-5-iodo-6-methyl-1-((2-(trimethylsilyl)ethoxy)methyl)-1H-indazole

A solution of the title compound from step 3 (13.5 g, 38.0 mmol) in THF (135 mL) cooled to 0° C. was added t-BuOK (10.6 g, 95.0 mmol) followed by SEM-C1 (14.6 g, 87.4 mmol, 15.4 mL) dropwise at 0-5° C. The mixture was then warmed to rt and stirred for 30 min. The reaction mixture was quenched by addition H₂O (200 mL) and then extracted with EtOAc (2×100 mL). The combined organic layers were washed with brine (100 mL), dried over anhydrous Na₂SO₄, filtered and concentrated under reduced pressure. The crude product was triturated with 75 mL hexanes at 0° C. for 30 min to afford the title compound (26.0 g, 51.9 mmol, 68.2% yield) as a white solid. ¹H NMR: (400 MHz, DMSO-d₆) δ 7.82 (s, 1H), 5.59 (s, 2H), 3.51 (t, 2H), 2.64 (s, 3H), 0.80 (t, 2H), −0.10 (s, 9H).

Step 5: Synthesis of 4-bromo-3-fluoro-6-methyl-1-((2-(trimethylsilyl)ethoxy)methyl)-5-vinyl-1H-indazole

A mixture of the title compound from step 4 (5.00 g, 10.30 mmol), potassium trifluoro(vinyl)borate (2.07 g, 15.5 mmol), AntPhos (763 mg, 2.06 mmol), palladium(ii) acetate (231 mg, 1.03 mmol) and potassium phosphate (6.56 g, 30.9 mmol) in water (10 mL) and toluene (50 mL) was degassed and purged with N₂ (3 times), then stirred at 100° C. for 12 h under N₂. The reaction mixture was diluted with water (50 mL) and extracted with EtOAc (3×30 mL). The combined organic layers were washed with brine (50 mL), dried over anhydrous Na₂SO₄, filtered and concentrated under reduced pressure. The residue was then purified by column chromatography (SiO₂, hexanes:EtOAc=1/0 to 15/1) to give the title compound (360 mg, 841 μmol, 8% yield) as a yellow oil. ¹H NMR (400 MHz, DMSO-d6) δ 7.67 (s, 1H), 6.69 (dd, 1H), 5.70 (dd, 1H), 5.59 (s, 2H), 5.45 (dd, 1H), 3.50 (d, 2H), 2.44 (s, 3H), 0.82-0.77 (m, 2H), −0.09-−0.10 (m, 9H)

Step 6: Synthesis of 4-bromo-3-fluoro-6-methyl-1-((2-(trimethylsilyl)ethoxy)methyl)-1H-indazole-5-carbaldehyde

To a solution of the title compound from step 5 (1.24 g, 3.22 mmol) in water (12.5 mL) and anhydrous tetrahydrofuran (12.5 mL) was added sodium periodate (2.06 g, 9.65 mmol, 535 μL), potassium osmate dihydrate (119 mg, 322 μmol) and 2,6-lutidine (690 mg, 6.44 mmol, 750 μL). The mixture was stirred at 50° C. for 2 h then quenched by sodium sulfite aqueous solution (30 mL), and extracted with EtOAc (3×30 mL). The combined organic layers were washed with brine (3×30 mL), dried over Na₂SO₄, filtered and concentrated under reduced pressure. The residue was purified by column chromatography (SiO₂,hexanes/EtOAc=100/1 to 8/1) to give compound the title compound (740 mg, 1.91 mmol, 59% yield) as a white solid. 1H NMR (400 MHz, MeOD): δ ppm 10.59 (s, 1H), 7.54 (s, 1H), 5.63 (d, 2H), 3.65-3.60 (m, 2H), 2.71 (s, 3H), 0.92-0.87 (m, 2H), −0.04 (s, 9H).

Step 7: Synthesis of 4-bromo-5-(difluoromethyl)-3-fluoro-6-methyl-1-((2-(trimethylsilyl)ethoxy)methyl)-1H-indazole

To a solution of the title compound from step 6 (300 mg, 775 μmol) in DCM (3 mL) degassed and purged with N₂ for 3 times was added DAST (624 mg, 3.87 mmol, 512 μL) at −78° C. under N₂. The mixture was then warmed to rt and stirred for 12 h. The mixture was then quenched by addition of aqueous NaHCO₃ solution (20 mL) and extracted with EtOAc (3×20 mL). The combined organic layers were washed with brine (3×20 mL), dried over Na₂SO₄, filtered and concentrated under reduced pressure. The residue was purified by column chromatography (SiO₂, hexanes/EtOAc=20/1 to 10/1) to give compound the title compound (280 mg, 684 μmol, 88% yield) as a white oil. ¹H NMR (400 MHz, MeOD): δ ppm 7.63-7.29 (m, 2H), 5.62 (s, 2H), 3.65-3.57 (m, 2H), 2.69 (s, 3H), 0.88 (t, 2H), −0.05 (s, 9H).

Step 8: Synthesis of tert-butyl (3-cyano-4-(5-(difluoromethyl)-3-fluoro-6-methyl-1-((2-(trimethylsilyl)ethoxy)methyl)-1H-indazol-4-yl)-7-fluorobenzo[b]thiophen-2-yl)carbamate

A mixture of the title compound from step 7 (230 mg, 562 μmol), Intermediate 9 (454 mg, 1.12 mmol), DPEphos PdCl₂ (40.2 mg, 56.2 μmol) and Cs₂CO₃ (549 mg, 1.69 mmol) in dioxane (5 mL) degassed and purged with N₂ (3 times) was stirred at 100° C. for 12 h under N₂. The mixture was then diluted with water (10 mL) and extracted with EtOAc (3×10 mL). The combined organic layers were washed with brine (3×10 mL), dried over Na₂SO₄, filtered and concentrated under reduced pressure. The residue was purified by column chromatography (SiO2, hexanes/EtOAc=100/1 to 8/1) to give compound the title compound (150 mg, 161 μmol, 34% yield) as a yellow oil. ¹H NMR (400 MHz, CDCl₃): δ ppm 7.74 (br s, 1H), 7.51 (s, 1H), 7.35-7.31 (m, 1H), 7.20-7.12 (m, 1H), 6.61-6.30 (m, 1H), 5.70-5.53 (m, 2H), 3.63-3.56 (m, 2H), 2.78 (s, 3H), 1.59 (s, 9H), 0.98-0.91 (m, 2H), 0.00 (s, 9H).

Step 9: Synthesis of tert-butyl (3-cyano-4-(5-(difluoromethyl)-3-fluoro-6-methyl-1H-indazol-4-yl)-7-fluorobenzo[b]thiophen-2-yl)carbamate

A solution of the title compound from step 8 (150 mg, 242 μmol) in TBAF (1 M THE solution, 4.5 mL) was stirred at 80° C. for 2 h then diluted with water (10 mL) and extracted with EtOAc (3×10 mL). The combined organic layers were washed with brine (3×10 mL), dried over Na₂SO₄, filtered and concentrated under reduced pressure. The residue was purified by column chromatography (SiO₂, hexanes/EtOAc=100/1 to 4/1) to give the title compound (110 mg, 224 μmol, 92% yield) as a white solid. LCMS m/z=493.3 [M+H]+, ¹H NMR (400 MHz, 6d-DMSO): δ ppm 12.78 (s, 1H), 11.61 (br s, 1H), 7.50 (s, 1H), 7.36 (d, 2H), 6.75-6.41 (m, 1H), 2.64 (s, 3H), 1.50 (s, 9H).

### Example 1

### (S)-2-amino-4-(5-chloro-3-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (R)-2-amino-4-(5-chloro-3-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

### And Example 2

### (R)-2-amino-4-(5-chloro-3-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (S)-2-amino-4-(5-chloro-3-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

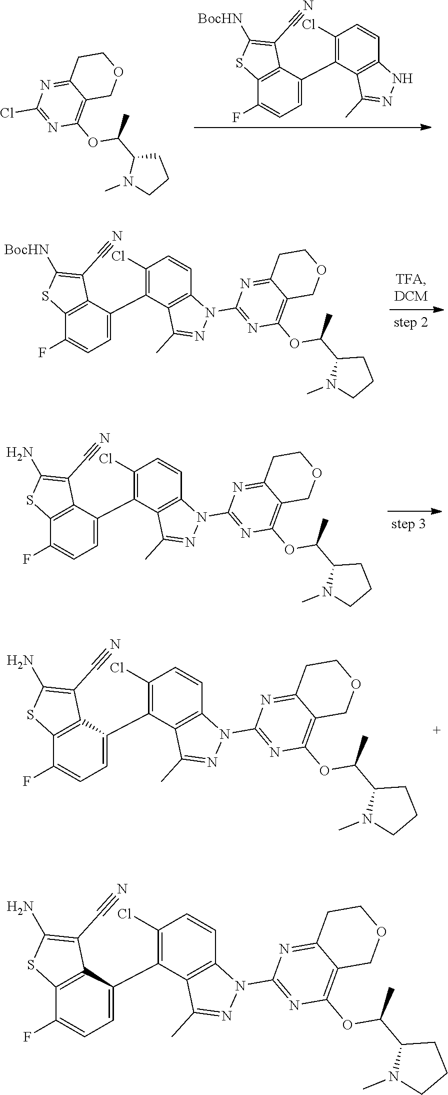

Step 1: Synthesis of tert-butyl (4-(5-chloro-3-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-3-cyano-7-fluorobenzo[b]thiophen-2-yl)carbamate

To a stirred solution of Intermediate 1 (130 mg, 436 μmol) and Intermediate 15 (238 mg, 523 μmol) in dioxane (3 mL) were added BINAP (13.5 mg, 21.8 μmol), BINAP Pd G3 (21.6 mg, 21.8 μmol) and Cs₂CO₃ (284 mg, 872 μmol) under N₂ and the reaction mixture was stirred for 12 h at 100° C. Water (10 mL) was added and the mixture extracted with DCM (5 mL×3). The combined organic layers were dried over Na₂SO₄ and concentrated. The crude product was purified by prep-TLC with DCM:MeOH=10:1 to give the title compound (120 mg, 38%) as a yellow solid. LCMS m/z=719 [M+H]⁺

Step 2: Synthesis of 2-amino-4-(5-chloro-3-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

To a stirred solution of the title compound from step 1 (120 mg, 167 μmol) in DCM (3 mL) was added TFA (1 mL) and the reaction mixture was stirred for 2 h at rt and concentrated. The pH of the residue was adjusted to 7 using sat. aq. NaHCO₃ and the mixture was extracted with DCM (10 mL×3). The combined organic layers were dried over Na₂SO₄ and concentrated. The crude product was purified by Prep-TLC with DCM:MeOH=5:1 to give the title compound (70 mg, 67%) as a yellow solid. LCMS m/z=619 [M+H]+

Step 3: Synthesis of (S)-2-amino-4-(5-chloro-3-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile and (R)-2-amino-4-(5-chloro-3-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

The title compound from step 2 was purified by prep-HPLC (Method A2, Gradient: 10% B to 41% B in 15 min) to give Peak 1, Example 1 (21.1 mg 26%) as a white solid and Peak 2, Example 2 (28.4 mg, 33%) as a white solid.

Example 1: LCMS: m/z=618 [M+H]⁺, ¹H NMR (400 MHz, DMSO-d₆) δ 8.68 (d, 1H), 8.04 (s, 2H), 7.70 (d, 1H), 7.26 (dd, 1H), 7.16 (dd, 1H), 5.43 (p, 1H), 4.65-4.51 (m, 2H), 4.00 (t, 2H), 2.98 (ddd, 1H), 2.90 (t, 2H), 2.66 (dt, 1H), 2.39 (s, 3H), 2.22 (td, 1H), 1.92-1.58 (m, 7H), 1.33 (d, 3H).

Example 2: LCMS: m/z=618 [M+H]⁺, ¹H NMR (400 MHz, DMSO-d₆) δ 8.69 (d, 1H), 8.05 (s, 2H), 7.70 (d, 1H), 7.26 (dd, 1H), 7.21-7.12 (m, 1H), 5.46 (p, 1H), 4.65-4.51 (m, 2H), 4.00 (t, 2H), 2.97 (ddd, 1H), 2.93-2.87 (m, 2H), 2.66 (dt, 1H), 2.39 (s, 3H), 2.26-2.15 (m, 1H), 1.87-1.63 (m, 7H), 1.33 (d, 3H).

### Example 3

### (S)-2-amino-4-(5-chloro-3-fluoro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (R)-2-amino-4-(5-chloro-3-fluoro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

### And Example 4

### (R)-2-amino-4-(5-chloro-3-fluoro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (S)-2-amino-4-(5-chloro-3-fluoro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

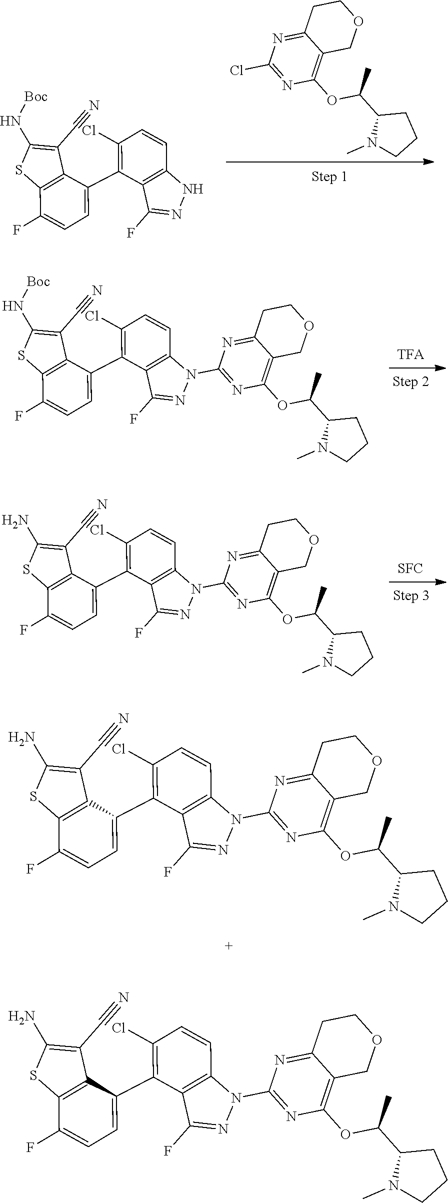

Step 1: Synthesis of tert-butyl (4-(5-chloro-3-fluoro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-3-cyano-7-fluorobenzo[b]thiophen-2-yl)carbamate

A mixture of Intermediate 14 (140 mg, 304 μmol), Intermediate 1 (90.5 mg, 304 μmol), EPhos Pd G4 (27.9 mg, 30.4 μmol) and Cs₂CO₃ (198 mg, 608 μmol) in dioxane (3 mL) was degassed and purged with N₂. The reaction mixture was stirred at 100° C. for 2 h under N₂, then cooled, filtered and concentrated under reduced pressure. The crude product was purified by reverse-phase HPLC (0.1% formic acid) to afford the title compound (200 mg, 91%) as a white solid. LCMS m/z=721 [M+H]+

Step 2: Synthesis of 2-amino-4-(5-chloro-3-fluoro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

To a solution of the title compound from step 1 (180 mg, 249 μmol) in DCM (4 mL) was added TFA (0.8 mL) and the reaction mixture was stirred at 25° C. for 1 h. The reaction was quenched with NH₄OH (1 mL), then diluted with water (6 mL) and extracted with DCM (10 mL×2). The combined organic layers were concentrated under reduced pressure. The residue was purified by prep-HPLC (Method A4, gradient: 23%-53% B over 10 min) to afford the title compound (100 mg, 63%) as a white solid. LCMS m/z=621 [M+H]+

Step 3: Synthesis of (S)-2-amino-4-(5-chloro-3-fluoro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile and (R)-2-amino-4-(5-chloro-3-fluoro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

The title compound from step 2 (100 mg, 161 μmol) was purified by prep-SFC (column: DAICEL CHIRALPAK IC (250 mm*30 mm, 10 μm); mobile phase: [CO₂-MeOH (0.1% NH₃H₂O)]; Gradient: 40% B isocratic elution mode) to afford Peak 1, Example 3 (20.7 mg, 21%) as a white solid and Peak 2. Peak 2 was further purified by prep-HPLC (column: Phenomenex luna C18 250*50 mm*15 μm; mobile phase: [water (formic acid)-MeCN]; gradient: 22%-52% B over 9 min) to give Example 4 (16.8 mg, 16%) as a white solid.

Example 3: LCMS m/z=622 [M+H]+. 1H NMR (400 MHz, DMSO-d₆) δ ppm 8.75-8.64 (m, 1H), 8.07 (s, 2H), 7.82 (d, 1H), 7.40-7.28 (m, 1H), 7.15 (t, 1H), 5.41 (t, 1H), 4.64-4.49 (m, 2H), 3.99 (t, 2H), 3.02-2.84 (m, 3H), 2.70-2.58 (m, 1H), 2.37 (s, 3H), 2.25-2.15 (m, 1H), 1.86-1.60 (m, 4H), 1.32 (d, 3H).

Example 4: LCMS m/z=622 [M+H]+1H NMR (400 MHz, DMSO-d₆) δ ppm 8.75-8.70 (m, 1H), 8.14 (s, 1H), 8.08 (s, 2H), 7.86 (d, 1H), 7.35-7.30 (m, 1H), 7.16 (t, 1H), 5.46-5.37 (m, 1H), 4.68-4.56 (m, 2H), 4.01 (t, 2H), 3.23 (s, 1H), 2.92 (t, 2H), 2.61 (s, 3H), 2.53-2.51 (m, 1H), 2.49-2.48 (m, 1H), 2.07-1.96 (m, 1H), 1.83 (d, 3H), 1.40 (d, 3H).

### Example 5

### (R)-2-amino-4-(5,6-dichloro-3-fluoro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile formate or (S)-2-amino-4-(5,6-dichloro-3-fluoro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile formate

### And Example 6

### (S)-2-amino-4-(5,6-dichloro-3-fluoro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile formate or (R)-2-amino-4-(5,6-dichloro-3-fluoro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile formate

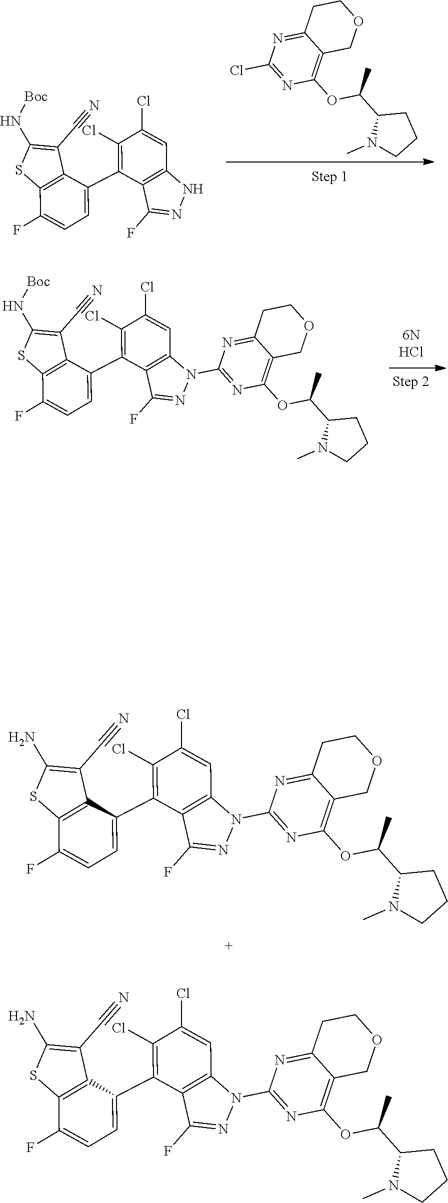

Step 1: Synthesis of tert-butyl (3-cyano-4-(5,6-dichloro-3-fluoro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophen-2-yl)carbamate

A mixture of Intermediate 11 (100 mg, 202 μmol), Intermediate 1 (60.0 mg, 202 μmol), Cs₂CO₃ (132 mg, 404 μmol) and EPhos Pd G4 (46.0 mg, 50.0 μmol) in dioxane (2 mL) was degassed and purged with N₂ and the reaction mixture was stirred at 60° C. for 3 h under N₂. The reaction mixture was diluted with water (10 mL) and extracted with EtOAc (10 mL×3). The combined organic layers were dried over Na₂SO₄, filtered and concentrated in vacuo. The residue was purified by reverse-phase HPLC (MeCN:H₂O (0.1% TFA)=0%~60%) to give the title compound (60.0 mg, 39%) as a yellow oil. LCMS m/z=756 [M+H]+

Step 2: Synthesis of (R)-2-amino-4-(5,6-dichloro-3-fluoro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile formate and (S)-2-amino-4-(5,6-dichloro-3-fluoro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile formate

To a solution of the title compound from step 1 (56.0 mg, 74.0 μmol) in dioxane (4 mL) was added HCl (6N, 4 mL) and the reaction mixture was stirred at 50° C. for 20 min. The reaction mixture was basified to pH 8 using NaHCO₃ and extracted with EtOAc (20 mL×3). The combined organic layers were dried over Na₂SO₄, filtered and concentrated in vacuo. The residue was purified by prep-HPLC (Method A3, gradient:25%-55% B over 10.0 min) to give Peak 1, Example 5 (2.30 mg, 4.4%) as a yellow solid and Peak 2, Example 6 (3.70 mg, 7.12%) as a yellow solid.

Example 5. LCMS m/z=656 [M+H]+ 1H NMR (400 MHz, DMSO-d₆) δ ppm 8.98 (d, 1H), 8.45 (s, 1H), 8.15 (s, 2H), 7.42-7.36 (m, 1H), 7.22-7.14 (m, 1H), 5.44-5.38 (m, 1H), 4.66-4.54 (m, 2H), 4.05-3.96 (m, 2H), 3.02-2.88 (m, 3H), 2.66-2.61 (m, 1H), 2.38 (s, 3H), 2.26-2.20 (m, 1H), 1.91-1.82 (m, 1H), 1.77-1.65 (m, 3H), 1.34 (d, 3H).

Example 6, LCMS m/z=656 [M+H]+ 1H NMR (400 MHz, DMSO-d₆) δ ppm 8.97 (d, 1H), 8.42 (s, 1H), 8.15 (s, 2H), 7.42-7.35 (m, 1H), 7.22-7.14 (m, 1H), 5.44-5.37 (m, 1H), 4.65-4.52 (m, 2H), 4.03-3.96 (m, 2H), 2.99-2.88 (m, 3H), 2.68-2.60 (m, 1H), 2.37 (s, 3H), 2.25-2.18 (m, 1H), 1.91-1.80 (m, 1H), 1.78-1.62 (m, 3H), 1.34 (d, 3H).

### Example 7

### (S)-2-amino-4-(3-chloro-5-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (R)-2-amino-4-(3-chloro-5-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

### And Example 8

### (R)-2-amino-4-(3-chloro-5-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (S)-2-amino-4-(3-chloro-5-methyl-1-(4-((S)-1-((S)-1-methylpyrrolidin-2-yl)ethoxy)-7,8-dihydro-5H-pyrano[4,3-d]pyrimidin-2-yl)-1H-indazol -4-yl)-7-fluorobenzo[b]thiophene-3-carbonitrile

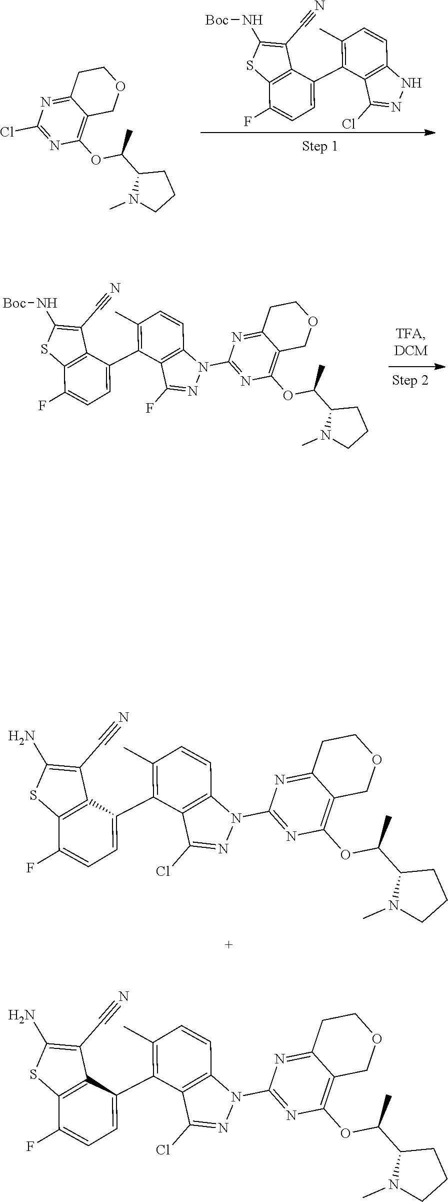

Step 1: Synthesis of tert-butyl (4-(3-chloro-5-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-3-cyano-7-fluorobenzo[b]thiophen-2-yl)carbamate

To a solution of Intermediate 1 (130 mg, 437 μmol), Intermediate 13 (199 mg, 437 μmol) in dioxane (5 mL) was added EPhos Pd G4 (40.1 mg, 43.7 μmol) and Cs₂CO₃ (284 mg, 873 μmol) and the reaction mixture was stirred at 100° C. for 1 h. Water (3 mL) was added to the cooled reaction and the mixture was extracted with EtOAc (15 mL×3). The combined organic extracts were concentrated, and the residue was purified by prep-HPLC (Method A4, gradient:40%-70% B over 20 min) to give the title compound (120 mg, 38%) as white solid. LCMS m/z=719 [M+H]+

Step 2: Synthesis of (R)-2-amino-4-(3-chloro-5-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (S)-2-amino-4-(3-chloro-5-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

To a solution of the title compound of step 1 (120 mg, 167 μmol) in DCM (2 mL) was added TFA (0.7 mL) and the mixture was stirred at 25° C. for 0.5 h. The reaction was evaporated under reduced pressure and the residue was purified by prep-HPLC (Method A4, gradient:18%-48% B over 10 min) to give Peak 1 and Peak 2. Peak 1 was purified by prep-HPLC (Method A4, gradient:28%-58% B over 20 min) to give Example 7 (24.0 mg, 23%) as white solid. LCMS m/z=619 [M+H]^{+ 1}H NMR (400 MHz, DMSO-d₆) δ ppm 10.13 (s, 1H), 8.67 (d, 1H), 7.96 (s, 2H), 7.65 (d, 1H), 7.19-7.11 (m, 1H), 5.48-5.43 (m, 1H), 4.68-4.65 (m, 2H), 4.05-4.01 (m, 2H), 3.80-3.79 (m, 1H), 3.40-3.15 (m, 2H), 2.97-2.96 (m, 5H), 2.35-2.25 (m, 1H), 2.15-2.09 (m, 4H), 1.97-1.95 (m, 2H), 1.51 (d, 3H)

Peak 2 was purified by prep-HPLC (Method A4, gradient:38%-68% B over 20 min) and further purified by SFC (column: DAICEL CHIRALPAK AD (250 mm*30 mm, 10 um); mobile phase: [CO₂-ACN/i-PrOH (0.1% NH₃H₂O)]; gradient 60% B, isocratic elution mode) to give Example 8 (18.8 mg, 51%) as white solid. LCMS m/z=619 [M+H]+ 1H NMR (400 MHz, DMSO-d₆) δ ppm 8.65 (d, 1H), 7.62 (d, 2H), 7.19-7.11 (m, 2H), 5.47-5.44 (m, 1H), 4.64-4.54 (m, 2H), 4.02-3.99 (m, 2H), 3.00 (m, 1H), 2.92 (m, 2H), 2.71 (m, 1H), 2.41 (s, 3H), 2.26 (m, 1H), 2.12 (s, 3H), 1.85-1.71 (m, 4H), 1.35 (d, 3H)

### Example 9

### (S)-2-amino-4-(5-chloro-3-fluoro-1-(4-((S)-1-((S)-1-methylpyrrolidin-2-yl)ethoxy)-5,7-dihydrofuro[3,4-d]pyrimidin-2-yl)-1H-indazol-4-yl)-7-fluorobenzo[b]thiophene-3-carbonitrile or (R)-2-amino-4-(5-chloro-3-fluoro-1-(4-((S)-1-((S)-1-methylpyrrolidin-2-yl)ethoxy)-5,7-dihydrofuro[3,4-d]pyrimidin-2-yl)-1H-indazol-4-yl)-7-fluorobenzo[b]thiophene-3-carbonitrile

### And Example 10

### (R)-2-amino-4-(5-chloro-3-fluoro-1-(4-((S)-1-((S)-1-methylpyrrolidin-2-yl)ethoxy)-5,7-dihydrofuro[3,4-d]pyrimidin-2-yl)-1H-indazol-4-yl)-7-fluorobenzo[b]thiophene-3-carbonitrile or (S)-2-amino-4-(5-chloro-3-fluoro-1-(4-((S)-1-((S)-1-methylpyrrolidin-2-yl)ethoxy)-5,7-dihydrofuro[3,4-d]pyrimidin-2-yl)-1H-indazol-4-yl)-7-fluorobenzo[b]thiophene-3-carbonitrile

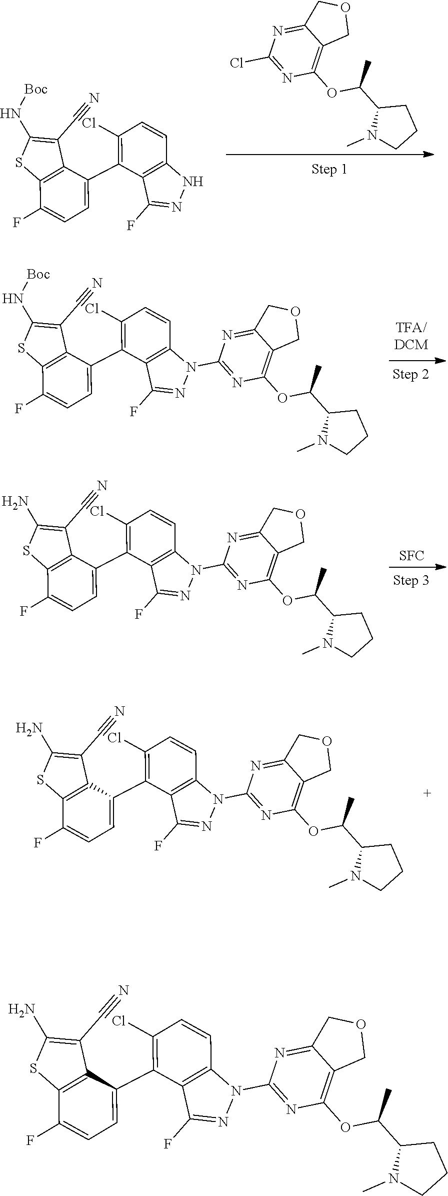

Step 1: Synthesis of tert-butyl (4-(5-chloro-3-fluoro-1-(4-((S)-1-((S)-1-methylpyrrolidin-2-yl)ethoxy)-5,7-dihydrofuro[3,4-d]pyrimidin-2-yl)-1H-indazol-4-yl)-3-cyano-7-fluorobenzo[b]thiophen-2-yl)carbamate

The title compound was obtained as a yellow solid, 120 mg, 74%, from Intermediate 2 and Intermediate 14, following a similar procedure to that described in Example 7 and 8, step 1. LCMS m/z=708 [M+H]+

Step 2: Synthesis of 2-amino-4-(5-chloro-3-fluoro-1-(4-((S)-1-((S)-1-methylpyrrolidin-2-yl)ethoxy)-5,7-dihydrofuro[3,4-d]pyrimidin-2-yl)-1H-indazol-4-yl)-7-fluorobenzo[b]thiophene-3-carbonitrile

To a solution of the title compound from step 1 (100 mg, 141 μmol) in DCM (1.5 mL) was added TFA (767 mg, 6.73 mmol) and the reaction mixture was stirred at 25° C. for 0.5 h. The reaction mixture was concentrated in vacuo and the residue was purified by prep-HPLC (Method A4, gradient:28%-58% B over 15.0 min) to give the title compound (60.0 mg, 67%) as white solid. LCMS m/z=608 [M+H]+

Step 3: Synthesis of (S)-2-amino-4-(5-chloro-3-fluoro-1-(4-((S)-1-((S)-1-methylpyrrolidin-2-yl)ethoxy)-5,7-dihydrofuro[3,4-d]pyrimidin-2-yl)-1H-indazol-4-yl)-7-fluorobenzo[b]thiophene-3-carbonitrile and (R)-2-amino-4-(5-chloro-3-fluoro-1-(4-((S)-1-((S)-1-methylpyrrolidin-2-yl)ethoxy)-5,7-dihydrofuro[3,4-d]pyrimidin-2-yl)-1H-indazol-4-yl)-7-fluorobenzo[b]thiophene-3-carbonitrile

The title compound from step 2 was further purified by SFC (column: DAICEL CHIRALCEL OD (250 mm*30 mm, 10 um), mobile phase: [CO₂-MeOH (0.1% NH₃·H₂O)], gradient:50% B, isocratic elution mode) to give compound Peak 1, Example 9 (18.1 mg, 29%) as a yellow solid and Peak 2, Example 10 (17.4 mg, 29%) as a yellow solid.

Example 9. LCMS m/z=608 [M+H]+ 1H NMR (400 MHz, DMSO-d₆) δ ppm 8.74-8.66 (m, 1H), 8.09 (s, 2H), 7.88 (d, 1H), 7.38-7.30 (m, 1H), 7.17 (t, 1H), 5.51-5.42 (m, 1H), 5.04 (s, 4H), 2.99-2.93 (m, 1H), 2.68-2.64 (m, 1H), 2.38 (s, 3H), 2.25-2.18 (m, 1H), 1.89-1.81 (m, 1H), 1.76-1.65 (m, 3H), 1.37 (d, 3H).

Example 10. LCMS m/z=608 [M+H]+. 1H NMR (400 MHz, DMSO-d₆) δ ppm 8.73-8.65 (m, 1H), 8.09 (s, 2H), 7.88 (d, 1H), 7.38-7.30 (m, 1H), 7.21-7.12 (m, 1H), 5.50-5.38 (m, 1H), 5.12-5.00 (m, 4H), 3.01-2.93 (m, 1H), 2.68-2.62 (m, 1H), 2.38 (s, 3H), 2.26-2.17 (m, 1H), 1.90-1.80 (m, 1H), 1.77-1.63 (m, 3H), 1.36 (d, 3H).

### Example 11

### (S)-2-amino-4-(5-chloro-3-fluoro-6-methyl-1-(4-((S)-1-((S)-1-methylpyrrolidin-2-yl)ethoxy)-5,7-dihydrofuro[3,4-d]pyrimidin-2-yl)-1H-indazol-4-yl)-7-fluorobenzo[b]thiophene-3-carbonitrile or (R)-2-amino-4-(5-chloro-3-fluoro-6-methyl-1-(4-((S)-1-((S)-1-methylpyrrolidin-2-yl)ethoxy)-5,7-dihydrofuro[3,4-d]pyrimidin-2-yl)-1H-indazol-4-yl)-7-fluorobenzo[b]thiophene-3-carbonitrile

### And Example 12

### (R)-2-amino-4-(5-chloro-3-fluoro-6-methyl-1-(4-((S)-1-((S)-1-methylpyrrolidin-2-yl)ethoxy)-5,7-dihydrofuro[3,4-d]pyrimidin-2-yl)-1H-indazol-4-yl)-7-fluorobenzo[b]thiophene-3-carbonitrile or (S)-2-amino-4-(5-chloro-3-fluoro-6-methyl-1-(4-((S)-1-((S)-1-methylpyrrolidin-2-yl)ethoxy)-5,7-dihydrofuro[3,4-d]pyrimidin-2-yl)-1H-indazol-4-yl)-7-fluorobenzo[b]thiophene-3-carbonitrile

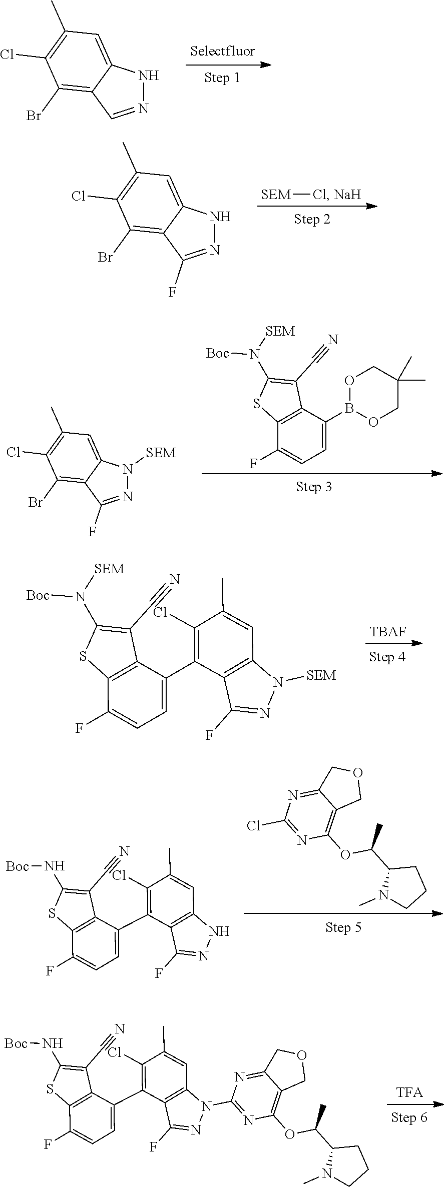

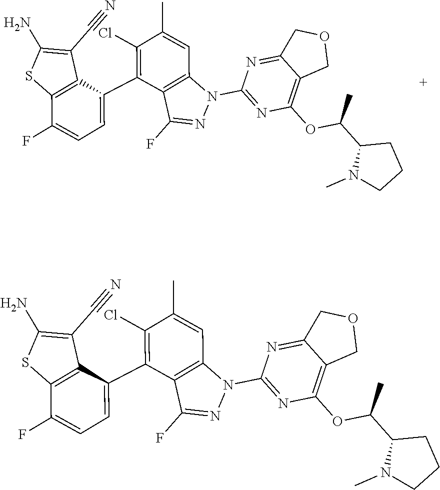

Step 1: Synthesis of 4-bromo-5-chloro-3-fluoro-6-methyl-1H-indazole

A mixture of 4-bromo-5-chloro-6-methyl-1H-indazole (1.0 g, 4.07 mmol) and Selectfluor (1.88 g, 5.30 mmol) in MeCN (20 mL) was stirred at 60° C. for 2 h under microwave irradiation. The reaction mixture was concentrated in vacuo and the residue was purified by prep HPLC (column: Princeton SFC DNP 250*50 mm*10 um, mobile phase: [Hexane-EtOH]; gradient:1%-40% B over 15.0 min) to give the title compound (260 mg, 12%) as a white solid. LCMS m/z=263 [M+H]+

Step 2: Synthesis of 4-bromo-5-chloro-3-fluoro-6-methyl-1-((2-(trimethylsilyl)ethoxy)methyl)-1H-indazole

The title compound was obtained as a white solid, 300 mg, 77%, from the title compound from step 1 and SEMCl, following a similar procedure to that described in Intermediate 11, step 2. 1H NMR (400 MHz, CDCl₃) δ ppm 7.33 (d, 1H), 5.53 (s, 2H), 3.59-3.50 (m, 2H), 2.57 (s, 3H), 0.93-0.82 (m, 2H), −0.03 (s, 9H).

Step 3: Synthesis of tert-butyl (4-(5-chloro-3-fluoro-6-methyl-1-((2-(trimethylsilyl)ethoxy)methyl)-1H-indazol-4-yl)-3-cyano-7-fluorobenzo[b]thiophen-2-yl) ((2-(trimethylsilyl)ethoxy)methyl)carbamate

The title compound was obtained as a white solid, 450 mg, 74%, from the title compound from step 2 and Intermediate 10 following a similar procedure to that described in Intermediate 14, step 3. 1H NMR (400 MHz, DMSO-d₆) δ ppm 8.04 (s, 1H), 7.68-7.64 (m, 2H), 5.72 (s, 2H), 5.17 (s, 2H), 3.68-3.64 (m, 2H), 3.61-3.56 (m, 2H), 2.60 (s, 3H), 1.42 (s, 9H), 1.00-0.96 (m, 2H), 0.93-0.87 (m, 2H), 0.03 (s, 9H), 0.00 (s, 9H).

Step 4: Synthesis of tert-butyl (4-(5-chloro-3-fluoro-6-methyl-1H-indazol-4-yl)-3-cyano-7-fluorobenzo[b]thiophen-2-yl)carbamate

A mixture of the title compound from step 3 (400 mg, 544 μmol) in TBAF (1 M, 4.0 mL) was pumped into a flow reactor (150° C.). The residence time of the flow reactor was 10 mins. The reaction mixture was collected in a bottle and on completion, was diluted with water (10 mL), then extracted with EtOAc (20 mL×3). The combined organic layers were washed with brine (30 mL), dried over Na₂SO₄, filtered and evaporated. The crude product was purified by reverse-phase HPLC (0.1% formic acid) to give the title compound (130 mg, 45%) as a yellow solid. LCMS m/z=474 [M+H]+

Step 5: Synthesis of tert-butyl (4-(5-chloro-3-fluoro-6-methyl-1-(4-((S)-1-((S)-1-methylpyrrolidin-2-yl)ethoxy)-5,7-dihydrofuro[3,4-d]pyrimidin-2-yl)-1H-indazol-4-yl)-3-cyano-7-fluorobenzo[b]thiophen-2-yl)carbamate

The title compound was obtained as a yellow solid, 80 mg, 50%, from the title compound from step 4 and Intermediate 2, following a similar procedure to that described in Example 3 and 4, step 1. LCMS m/z=722 [M+H]+

Step 6: Synthesis of (S)-2-amino-4-(5-chloro-3-fluoro-6-methyl-1-(4-((S)-1-((S)-1-methylpyrrolidin-2-yl)ethoxy)-5,7-dihydrofuro[3,4-d]pyrimidin-2-yl)-1H-indazol-4-yl)-7-fluorobenzo[b]thiophene-3-carbonitrile trifluoroacetate and (R)-2-amino-4-(5-chloro-3-fluoro-6-methyl-1-(4-((S)-1-((S)-1-methylpyrrolidin-2-yl)ethoxy)-5,7-dihydrofuro[3,4-d]pyrimidin-2-yl)-1H-indazol-4-yl)-7-fluorobenzo[b]thiophene-3-carbonitrile trifluoroacetate

The title compounds were obtained from the title compound from step 5, following a similar procedure to that described in Example 9 and 10, step 2.

Example 11, (30.7 mg, 45%) as white solid: LCMS m/z=622 [M+H]+ 1H NMR (400 MHz, DMSO-d₆) δ ppm 8.71 (s, 1H), 8.06 (s, 2H), 7.36-7.27 (m, 1H), 7.21-7.07 (m, 1H), 5.57-5.39 (m, 1H), 5.16-5.01 (m, 4H), 3.86-3.76 (m, 1H), 3.65-3.61 (m, 1H), 3.26-3.11 (m, 1H), 2.96 (d, 3H), 2.61 (s, 3H), 2.36-2.25 (m, 1H), 2.16-2.08 (m, 1H), 2.04-1.86 (m, 2H), 1.55 (d, 3H).

Example 12: (6.0 mg, 9%) as white solid. LCMS m/z=622 [M+H]+ 1H NMR (400 MHz, DMSO-d₆) δ ppm 8.69 (s, 1H), 8.06 (s, 2H), 7.37-7.27 (m, 1H), 7.22-7.06 (m, 1H), 5.52-5.39 (m, 1H), 5.10 (s, 4H), 3.86-3.73 (m, 1H), 3.59-3.53 (m, 1H), 3.22-3.12 (m, 1H), 2.95 (d, 3H), 2.61 (s, 3H), 2.36-2.20 (m, 1H), 2.17-2.02 (m, 1H), 2.00-1.82 (m, 2H), 1.54 (d, 3H).

### Example 13

### (R)-2-amino-4-(5-chloro-3,6-difluoro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile trifluoroacetate or (S)-2-amino-4-(5-chloro-3,6-difluoro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile trifluoroacetate

### And Example 14

### (S)-2-amino-4-(5-chloro-3,6-difluoro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile trifluoroacetate or (R)-2-amino-4-(5-chloro-3,6-difluoro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile trifluoroacetate

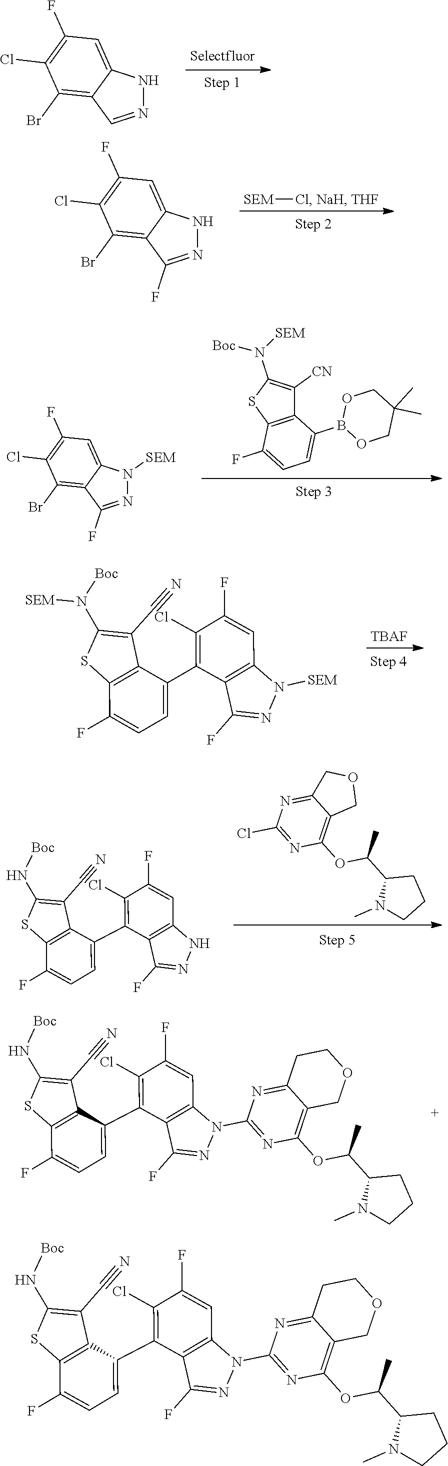

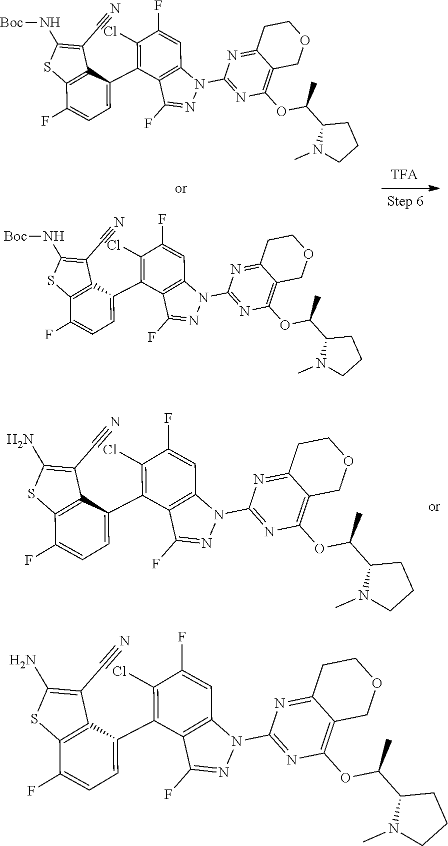

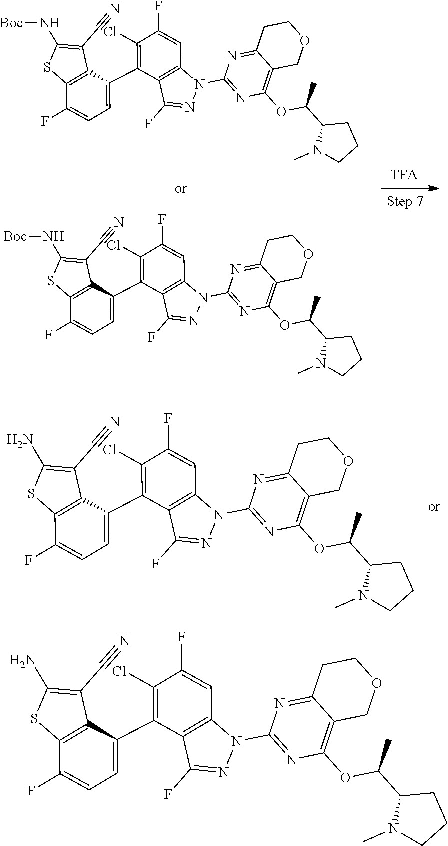

Step 1: Synthesis of 4-bromo-5-chloro-3,6-difluoro-1H-indazole

The title compound was obtained as a white solid, 200 mg, 10%, from 4-bromo-5-chloro-6-fluoro-1H-indazole and Selectfluor, following a similar procedure to that described in Intermediate 14, step 1. LCMS m/z=267 [M+H]+

Step 2: Synthesis of 4-bromo-5-chloro-3,6-difluoro-1-((2-(trimethylsilyl)ethoxy)methyl)-1H-indazole

The title compound was obtained as a white solid, 220 mg, 74%, from the title compound of step 1 and SEMCl, following a similar procedure to that described in Intermediate 11, step 2. 1H NMR (400 MHz, CDCl₃) δ ppm 7.28 (d, 1H), 5.52 (s, 2H), 3.62-3.49 (m, 2H), 0.93-0.84 (m, 2H), −0.03 (s, 9H).

Step 3: Synthesis of tert-butyl (4-(5-chloro-3,6-difluoro-1-((2-(trimethylsilyl)ethoxy)methyl)-1H-indazol-4-yl)-3-cyano-7-fluorobenzo[b]thiophen-2-yl) ((2-(trimethylsilyl)ethoxy)methyl)carbamate

The title compound was obtained as a yellow oil, 370 mg, 84%, from the title compound from step 2 and Intermediate 10, following a similar procedure to that described in Example 11 and 12, step 3. 1H NMR (400 MHz, CDCl₃) δ ppm 7.45-7.37 (m, 2H), 7.33-7.28 (m, 1H), 5.70-5.42 (m, 2H), 5.24-5.00 (m, 2H), 3.71-3.65 (m, 2H), 3.61-3.51 (m, 2H), 1.43 (s, 9H), 1.05-0.99 (m, 2H), 0.96-0.88 (m, 2H), 0.03-0.02 (m, 9H), −0.02 (s, 9H).

Step 4: Synthesis of tert-butyl (4-(5-chloro-3,6-difluoro-1H-indazol-4-yl)-3-cyano-7-fluorobenzo[b]thiophen-2-yl)carbamate

The title compound was obtained as a yellow solid, 100 mg, 45%, from the title compound from step 3, following a similar procedure to that described in Example 11 and 12, step 4. 1H NMR (400 MHz, DMSO-d₆) δ ppm 12.99 (s, 1H), 11.88-11.48 (m, 1H), 7.75-7.68 (m, 1H), 7.52-7.45 (m, 1H), 7.43-7.34 (m, 1H), 1.50 (s, 9H).

Step 5: Synthesis of tert-butyl ((R)-4-(5-chloro-3,6-difluoro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-3-cyano-7-fluorobenzo[b]thiophen-2-yl)carbamate and tert-butyl ((S)-4-(5-chloro-3,6-difluoro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-3-cyano-7-fluorobenzo[b]thiophen-2-yl)carbamate

A mixture of the title compound from step 4 (90.0 mg, 188 μmol), Intermediate 1 (67.2 mg, 226 μmol), EPhos Pd G4 (17.3 mg, 18.8 μmol) and Cs₂CO₃ (122 mg, 376 μmol) in dioxane (3 mL) was stirred at 100° C. for 2 h under N₂. The mixture was filtered and concentrated in vacuo. The mixture was purified by prep-HPLC (Method A4, gradient:40%-50% B over 10.0 min) to give Peak 1, enantiomer 1 (25.0 mg, 18%) as yellow solid and Peak 2, enantiomer 2 (40.0 mg, 26%) as yellow solid. LCMS m/z=739 [M+H]+

Step 6: Synthesis of (R)-2-amino-4-(5-chloro-3,6-difluoro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile trifluoroacetate or (S)-2-amino-4-(5-chloro-3,6-difluoro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile trifluoroacetate, Example 13.

The title compound was obtained as a yellow solid, 21.7 mg, 85%, from Peak 1, from step 5 following a similar procedure to that described in Example 9, step 2. LCMS m/z=640 [M+H]+ 1H NMR (400 MHz, DMSO-d₆) δ ppm 9.91 (s, 1H), 8.79-8.58 (m, 1H), 8.15 (s, 2H), 7.54-7.31 (m, 1H), 7.25-7.07 (m, 1H), 5.48-5.19 (m, 1H), 4.73-4.53 (m, 2H), 4.12-3.95 (m, 2H), 3.86-3.73 (m, 1H), 3.60-3.54 (m, 1H), 3.26-3.12 (m, 1H), 2.99 (s, 2H), 2.96 (d, 3H), 2.38-2.21 (m, 1H), 2.19-2.03 (m, 1H), 2.01-1.82 (m, 2H), 1.48 (d, 3H).

Step 7: Synthesis of (S)-2-amino-4-(5-chloro-3,6-difluoro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile trifluoroacetate or (R)-2-amino-4-(5-chloro-3,6-difluoro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile trifluoroacetate, Example 14.

The title compound was obtained as a yellow solid, 27.6 mg, 77%, from Peak 2, step 5, following the procedure described in Example 9, step 2. LCMS m/z=640 [M+H]+ 1H NMR (400 MHz, DMSO-d₆) δ ppm 9.90 (s, 1H), 8.79-8.59 (m, 1H), 8.15 (s, 2H), 7.48-7.29 (m, 1H), 7.25-7.10 (m, 1H), 5.47-5.31 (m, 1H), 4.76-4.55 (m, 2H), 4.15-3.93 (m, 2H), 3.85-3.71 (m, 1H), 3.63-3.58 (m, 1H), 3.25-3.11 (m, 1H), 3.05-2.99 (m, 2H), 2.96 (d, 3H), 2.35-2.21 (m, 1H), 2.16-2.02 (m, 1H), 2.01-1.82 (m, 2H), 1.47 (d, 3H).

### Example 15

### (R)-2-amino-4-(5-chloro-3-fluoro-6-hydroxy-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile formate and (S)-2-amino-4-(5-chloro-3-fluoro-6-hydroxy-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile formate

### And Example 16

### (S)-2-amino-4-(5-chloro-3-fluoro-6-hydroxy-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile formate and (R)-2-amino-4-(5-chloro-3-fluoro-6-hydroxy-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile formate

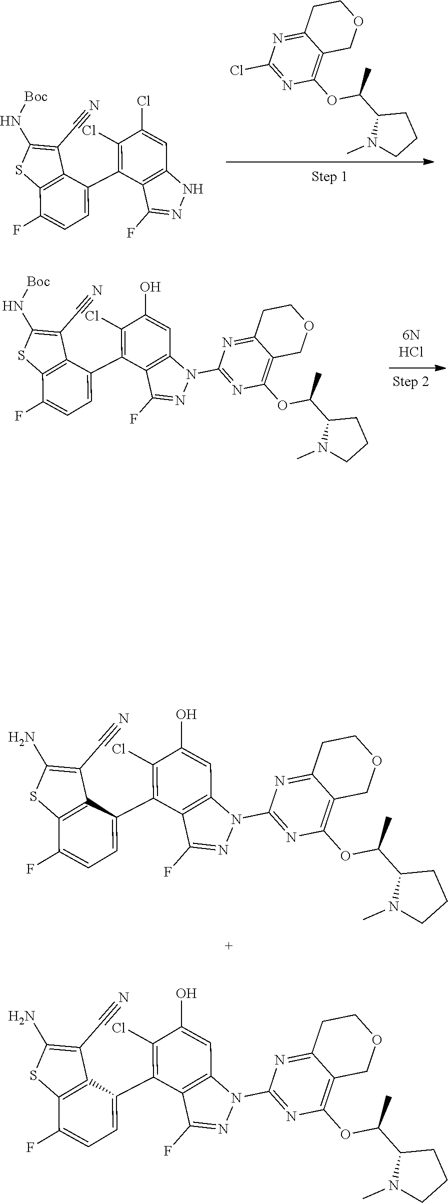

Step 1: Synthesis of tert-butyl (4-(5-chloro-3-fluoro-6-hydroxy-1-(4-((S)-1-((S)-1-methylpyrrolidin-2-yl)ethoxy)-7,8-dihydro-5H-pyrano[4,3-d]pyrimidin-2-yl)-1H-indazol-4-yl)-3-cyano-7-fluorobenzo[b]thiophen-2-yl)carbamate

A mixture of Intermediate 11 (76.0 mg, 153 μmol), Intermediate 1 (50.0 mg, 168 μmol) and Cs₂CO₃ (100 mg, 307 μmol), EPhos Pd G4 (14.0 mg, 15.3 μmol) in dioxane (3 mL) was degassed and purged with N₂, and the reaction mixture was stirred at 80° C. for 2 h. The reaction mixture was diluted with water (10 mL) and extracted with EtOAc (10 mL×3). The combined organic layers were dried over anhydrous Na₂SO₄, filtered and concentrated in vacuo. The residue was purified by reverse-phase HPLC (80 g, MeCN:H2O (0.1% TFA)=0%~60%) to give the title compound (40.0 mg, 35%) as yellow oil. LCMS m/z 738 [M+H]+

Step 2: Synthesis of (R)-2-amino-4-(5-chloro-3-fluoro-6-hydroxy-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile formate and (S)-2-amino-4-(5-chloro-3-fluoro-6-hydroxy-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile formate

To a solution of the title compound of step 1 (56.0 mg, 74.0 μmol) in dioxane (4 mL) was added HCl (6 M, 4 mL) and the reaction the mixture was stirred at 50° C. for 20 min. The reaction mixture was basified to pH 8 using aq. sat. NaHCO₃ and the mixture extracted with EtOAc (20 mL×3). The combined organic layers were dried over Na₂SO₄, filtered and concentrated under reduced pressure. The residue was purified by prep-HPLC (Method A4, gradient:25%-55% B over 10.0 min) to give Peak 1, Example 15 (2.30 mg, 4.4%) as yellow solid and Peak 2, Example 16 (3.70 mg, 7.1%) as a yellow solid.

Example 15: LCMS m/z=656 [M+H]+ 1H NMR (400 MHz, DMSO-d₆) δ ppm 8.98 (d, 1H), 8.45 (s, 1H), 8.15 (s, 2H), 7.42-7.36 (m, 1H), 7.22-7.14 (m, 1H), 5.44-5.38 (m, 1H), 4.66-4.54 (m, 2H), 4.05-3.96 (m, 2H), 3.02-2.88 (m, 3H), 2.66-2.61 (m, 1H), 2.38 (s, 3H), 2.26-2.20 (m, 1H), 1.91-1.82 (m, 1H), 1.77-1.65 (m, 3H), 1.34 (d, 3H).

Example 16: LCMS m/z=656 [M+H]+ 1H NMR (400 MHz, DMSO-d₆) δ ppm 8.97 (d, 1H), 8.42 (s, 1H), 8.15 (s, 2H), 7.42-7.35 (m, 1H), 7.22-7.14 (m, 1H), 5.44-5.37 (m, 1H), 4.65-4.52 (m, 2H), 4.03-3.96 (m, 2H), 2.99-2.88 (m, 3H), 2.68-2.60 (m, 1H), 2.37 (s, 3H), 2.25-2.18 (m, 1H), 1.91-1.80 (m, 1H), 1.78-1.62 (m, 3H), 1.34 (d, 3H).

### Example 17

### (R)-2-amino-4-(5-chloro-3-fluoro-6-(methoxymethyl)-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (S)-2-amino-4-(5-chloro-3-fluoro-6-(methoxymethyl)-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

### And Example 18

### (S)-2-amino-4-(5-chloro-3-fluoro-6-(methoxymethyl)-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (R)-2-amino-4-(5-chloro-3-fluoro-6-(methoxymethyl)-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

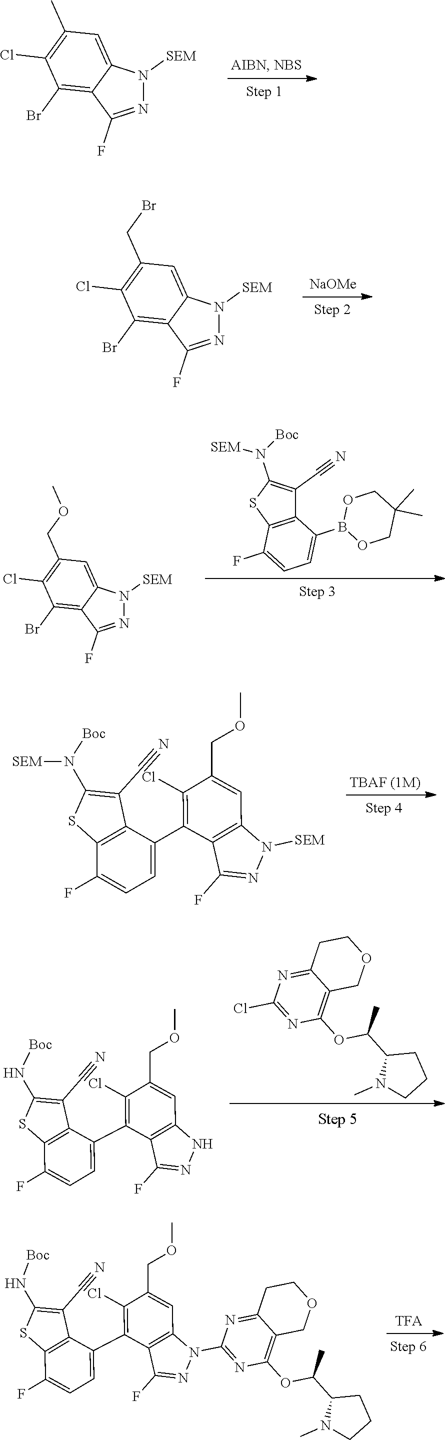

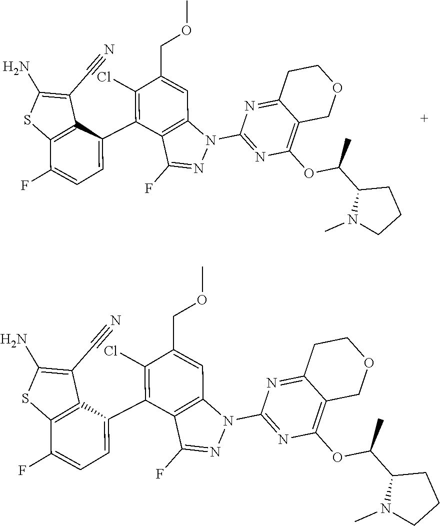

Step 1: Synthesis of 4-bromo-6-(bromomethyl)-5-chloro-3-fluoro-1-((2-(trimethylsilyl)ethoxy)methyl)-1H-indazole

To a solution of the compound from Example 11 and 12, step 2, (530 mg, 1.35 mmol) in 1,2-dichloroethane (5 mL) was added azodiisobutyronitrile (22.1 mg, 135 μmol) and N-bromosuccinimide (287 mg, 1.62 mmol) and the reaction mixture was stirred at 70° C. for 2 h. The reaction was diluted with water (50 mL) and then extracted with EtOAc (50 mL×3). The combined organic layers were washed with brine (50 mL), dried over Na₂SO₄, filtered and evaporated. The residue was purified by column chromatography (SiO₂, PE/EtOAc=1/0 to 20/1) to give the title compound (70.0 mg, 11%) as white solid. 1H NMR (400 MHz, CDCl₃) δ ppm 7.59 (s, 1H), 5.57 (s, 2H), 4.73 (s, 2H), 3.64-3.52 (m, 2H), 0.91 (d, 2H), −0.03 (s, 9H).

Step 2: Synthesis of 4-bromo-5-chloro-3-fluoro-6-(methoxymethyl)-1-((2-(trimethylsilyl)ethoxy)methyl)-1H-indazole

To a solution of the title compound from step 1 (100 mg, 212 μmol) in MeOH (0.5 mL) was added NaOMe (1.7 M in MeOH, 0.5 mL) and the reaction mixture was stirred at 25° C. for 0.5 h. The reaction was diluted with water (10 mL) and then extracted with EtOAc (20 mL×3). The combined organic layers were washed with brine (30 mL), dried over Na₂SO₄, filtered and evaporated. The residue was purified by prep-TLC (SiO₂, PE:EtOAc=10:1) to afford the title compound (70.0 mg, 78%) as white solid. 1H NMR (400 MHz, CDCl₃) δ ppm 7.67 (s, 1H), 5.60 (s, 2H), 4.67 (s, 2H), 3.69-3.47 (m, 5H), 1.01-0.82 (m, 2H), 0.00 (s, 9H).

Step 3: Synthesis of tert-butyl (4-(5-chloro-3-fluoro-6-(methoxymethyl)-1-((2-(trimethylsilyl)ethoxy)methyl)-1H-indazol-4-yl)-3-cyano-7-fluorobenzo[b]thiophen-2-yl) ((2-(trimethylsilyl)ethoxy)methyl)carbamate

The title compound was obtained as a white solid, 100 mg, 70%, from the title compound from step 2 and Intermediate 10, following the procedure described in Example 11 and 12, step 3. LCMS m/z=785 [M+Na]+

Step 4: Synthesis of tert-butyl (4-(5-chloro-3-fluoro-6-(methoxymethyl)-1H-indazol-4-yl)-3-cyano-7-fluorobenzo[b]thiophen-2-yl)carbamate

The title compound was obtained as a white solid, 40 mg, 64%, from the title compound from step 3, following the procedure described in Example 11 and 12, step 4. LCMS m/z=525 [M+Na]+

Step 5: Synthesis of tert-butyl (4-(5-chloro-3-fluoro-6-(methoxymethyl)-1-(4-((S)-1-((S)-1-methylpyrrolidin-2-yl)ethoxy)-7,8-dihydro-5H-pyrano[4,3-d]pyrimidin-2-yl)-1H-indazol-4-yl)-3-cyano-7-fluorobenzo[b]thiophen-2-yl)carbamate

The title compound was obtained as a yellow solid, 40 mg, 75%, from Intermediate 1 and the title compound from step 4, following a similar procedure to that described in Example 3 and 4, step 1, except prep-HPLC (Method A4, gradient:33%-63% B over 15.0 min) was used. LCMS m/z=766 [M+H]+

Step 6: Synthesis of (R)-2-amino-4-(5-chloro-3-fluoro-6-(methoxymethyl)-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile and (S)-2-amino-4-(5-chloro-3-fluoro-6-(methoxymethyl)-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

A mixture of the title compound from step 5 (35.0 mg, 45.7 μmol) in TFA (0.5 mL) and DCM (2 mL) was stirred at 20° C. for 0.5 h. The mixture was evaporated and the residue was purified by prep-HPLC (Method A4, gradient:20%-50% B over 15.0 min) to give a white solid (30.0 mg). This was purified by prep-SFC (column: DAICEL CHIRALPAK AD (250 mm*30 mm, 10 um), mobile phase: [CO₂-EtOH (0.1% NH₃·H₂O)]; gradient:50% B, isocratic elution mode) to give Peak 1, Example 17 (14.0 mg, 47%) as a white solid and Peak 2, Example 18 (12.9 mg, 43%) as a white solid.

Example 17. LCMS m/z=666 [M+H]+; 1H NMR (400 MHz, DMSO-d₆) δ ppm 8.88 (s, 1H), 8.06 (s, 2H), 7.34-7.30 (m, 1H), 7.16 (t, 1H), 5.44-5.36 (m, 1H), 4.69 (s, 2H), 4.64-4.54 (m, 2H), 4.01-3.98 (m, 2H), 3.52 (s, 3H), 2.98-2.95 (m, 1H), 2.89-2.85 (m, 2H), 2.65-2.61 (m, 1H), 2.38 (s, 3H), 2.25-2.20 (m, 1H), 1.94-1.90 (m, 1H), 1.75-1.67 (m, 3H), 1.37 (d, 3H).

Example 18. LCMS m/z=666 [M+H]+; 1H NMR (400 MHz, DMSO-d₆) δ ppm 8.88 (s, 1H), 8.06 (s, 2H), 7.34-7.30 (m, 1H), 7.16 (t, 1H), 5.46-5.42 (m, 1H), 4.69 (s, 2H), 4.64-4.57 (m, 2H), 4.01-3.98 (m, 2H), 3.53 (s, 3H), 3.02-2.96 (m, 1H), 2.89-2.86 (m, 2H), 2.65-2.61 (m, 1H), 2.36 (s, 3H), 2.26-2.21 (m, 1H), 1.95-1.90 (m, 1H), 1.77-1.66 (m, 3H), 1.38 (d, 3H).

### Example 19

### (S)-2-amino-4-(5-chloro-3-fluoro-1-(4-((S)-1-((2S,4R)-4-methoxy-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (R)-2-amino-4-(5-chloro-3-fluoro-1-(4-((S)-1-((2S,4R)-4-methoxy-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

### And Example 20

### (R)-2-amino-4-(5-chloro-3-fluoro-1-(4-((S)-1-((2S,4R)-4-methoxy-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (S)-2-amino-4-(5-chloro-3-fluoro-1-(4-((S)-1-((2S,4R)-4-methoxy-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

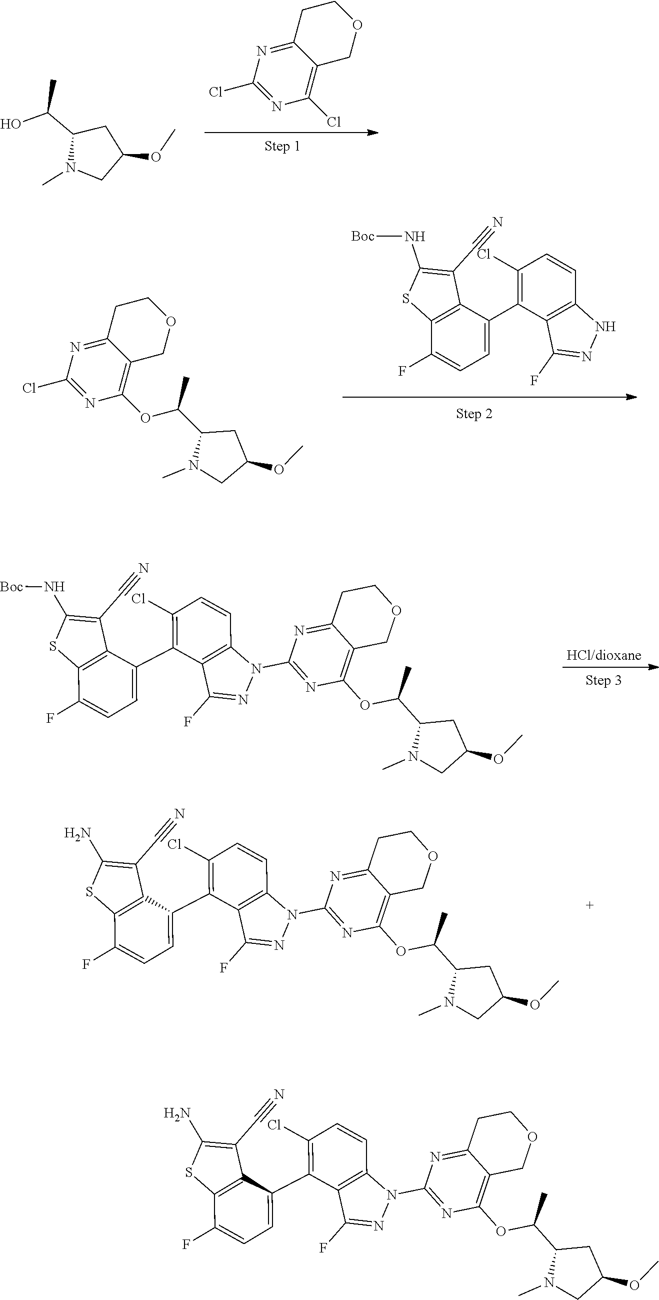

Step 1: Synthesis of 2-chloro-4-((S)-1-((2S,4R)-4-methoxy-1-methylpyrrolidin-2-yl)ethoxy)-7,8-dihydro-5H-pyrano[4,3-d]pyrimidine

The title compound was obtained as a white solid, 500 mg, 77%, from(S)-1-((2S,4R)-4-methoxy-1-methylpyrrolidin-2-yl)ethan-1-ol and Intermediate 1, step 1, following the procedure described in Intermediate 2. LCMS m/z=328 [M+H]+

Step 2: Synthesis of tert-butyl (4-(5-chloro-3-fluoro-1-(4-((S)-1-((2S,4R)-4-methoxy-1-methylpyrrolidin-2-yl)ethoxy)-7,8-dihydro-5H-pyrano[4,3-d]pyrimidin-2-yl)-1H-indazol-4-yl)-3-cyano-7-fluorobenzo[b]thiophen-2-yl)carbamate

The title compound was obtained as a yellow solid, 320 mg, 90%, from the title compound from step 1 and Intermediate 14, following a similar procedure to that described in Example 11 and 12, step-4 step3. LCMS m/z=752 [M+H]+

Step 3: Synthesis of (S)-2-amino-4-(5-chloro-3-fluoro-1-(4-((S)-1-((2S,4R)-4-methoxy-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile and (R)-2-amino-4-(5-chloro-3-fluoro-1-(4-((S)-1-((2S,4R)-4-methoxy-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

A solution of the title compound from step 2 (270 mg, 358 μmol) in 2M HCl in dioxane (10 mL) was stirred at 40° C. for 1 h. The reaction mixture was concentrated under reduced pressure and the residue was purified by reverse-phase HPLC (Method A4, gradient:25%-55% B over 10.0 min). The product was further purified by SFC (column: DAICEL CHIRALCEL OD (250 mm*30 mm, 10 um), mobile phase: [CO₂-MeOH (0.1% NH₃·H₂O)], Gradient: 45% B, isocratic elution mode) to give Peak 1, Example 19 (42.4 mg, 42%) as a white solid and Peak 2, Example 20 (30.1 mg, 30%) as white solid.

Example 19; LCMS m/z=652 [M+H]+ 1H NMR (400 MHz, DMSO-d₆) δ ppm 8.72 (d, 1H), 8.09 (s, 2H), 7.85 (d, 1H), 7.33 (dd, 1H), 7.16 (t, 1H), 5.42 (quin, 1H), 4.70-4.49 (m, 2H), 4.03-3.96 (m, 2H), 3.94-3.78 (m, 1H), 3.28 (s, 1H), 3.24-3.13 (m, 3H), 2.91 (s, 2H), 2.87-2.75 (m, 1H), 2.41 (s, 3H), 2.29-2.17 (m, 1H), 2.00-1.76 (m, 2H), 1.40-1.27 (m, 3H).

Example 20. LCMS m/z=652 [M+H]+ 1H NMR (400 MHz, DMSO-d₆) δ ppm 8.72 (dd, 1H), 8.09 (s, 2H), 7.87 (d, 1H), 7.32 (dd, 1H), 7.22-7.09 (m, 1H), 5.51-5.33 (m, 1H), 4.73-4.54 (m, 2H), 4.27-3.83 (m, 4H), 3.82-3.51 (m, 1H), 3.48-3.36 (m, 1H), 3.29 (s, 3H), 3.17-3.00 (m, 1H), 3.00-2.87 (m, 3H), 2.47-2.34 (m, 1H), 2.26-1.64 (m, 2H), 1.56-1.28 (m, 3H).

### Example 21

### (S)-2-amino-4-(5-chloro-3-fluoro-1-(4-((S)-1-((S)-1-(methyl-d3)pyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (R)-2-amino-4-(5-chloro-3-fluoro-1-(4-((S)-1-((S)-1-(methyl-d3)pyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

### And Example 22

### (R)-2-amino-4-(5-chloro-3-fluoro-1-(4-((S)-1-((S)-1-(methyl-d3)pyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (S)-2-amino-4-(5-chloro-3-fluoro-1-(4-((S)-1-((S)-1-(methyl-d3)pyrrolidin-2-yl)ethoxy)-7,8-dihydro-5H-pyrano[4,3-d]pyrimidin-2-yl)-1H-indazol -4-yl)-7-fluorobenzo[b]thiophene-3-carbonitrile

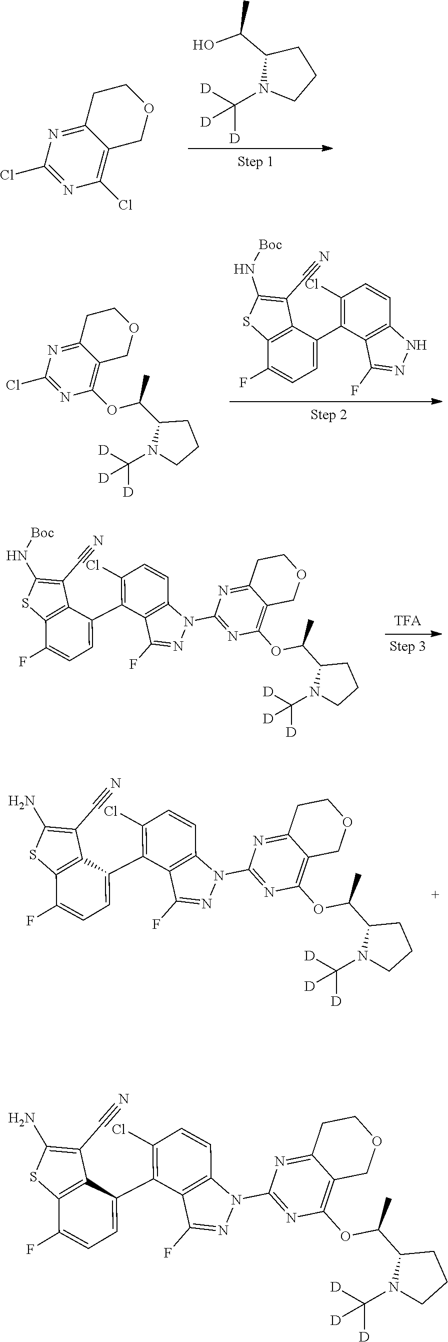

Step 1: Synthesis of 2-chloro-4-((S)-1-((S)-1-(methyl-d3)pyrrolidin-2-yl)ethoxy)-7,8-dihydro-5H-pyrano[4,3-d]pyrimidine trifluoroacetate

To a solution of Intermediate 22 (903 mg, 6.83 mmol) in dioxane (14 mL) was added KOtBu (919 mg, 8.19 mmol), the mixture was stirred at 25° C. for 30 mins, then Intermediate 1, step 1 (1.4 g, 6.83 mmol) was added and the reaction stirred for 15.5 h. The reaction mixture was diluted with water (20 mL) and extracted with EtOAc (20 mL×3). The combined organic layers were dried over Na₂SO₄, filtered and concentrated in vacuo. The residue was purified by prep-HPLC (column: Phenomenex luna C18 250×80 mm×10 μm; mobile phase: [H₂O (0.1% TFA)-MeCN]; gradient:1%-31% B over 20.0 min) to give the title compound (400 mg, 14%) as yellow solid. LCMS m/z=301 [M+H]+

Step 2: Synthesis of tert-butyl (4-(5-chloro-3-fluoro-1-(4-((S)-1-((S)-1-(methyl-d3) pyrrolidin-2-yl)ethoxy)-7,8-dihydro-5H-pyrano[4,3-d]pyrimidin-2-yl)-1H-indazol-4-yl)-3-cyano-7-fluorobenzo[b]thiophen-2-yl)carbamate

The title compound was obtained as a yellow solid, 320 mg, 42%, from the title compound from step 1 (380 mg, 916 μmol), and Intermediate 14, following a similar procedure to that described in Example 8, step 1. LCMS m/z=725 [M+H]+

Step 3: Synthesis of (S)-2-amino-4-(5-chloro-3-fluoro-1-(4-((S)-1-((S)-1-(methyl-d3) pyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile and (R)-2-amino-4-(5-chloro-3-fluoro-1-(4-((S)-1-((S)-1-(methyl-d3)pyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

To a solution of the title compound from step 2 (320 mg, 441 μmol) in DCM (3 mL) was added TFA (982 mg, 8.62 mmol) and the reaction was stirred at 25° C. for 4 h. The mixture was basified to pH 8 by addition of sat. aq. Na₂CO₃ and the mixture was extracted with EtOAc (30 mL×3). The combined organic phases were dried with Na₂SO₄, filtered and concentrated in vacuo. The residue was purified by prep-HPLC (Method A4, gradient:28%-58% B over 15.0 min) to give Peak 1, Example 21 (90.6 mg, 33%) and Peak 2, Example 22 (58.2 mg, 21%) as off-white solid.

Example 21, LCMS m/z=621 [M+H]+. 1H NMR (400 MHz, CD₃OD) δ ppm 8.87-8.72 (m, 1H), 7.79 (d, 1H), 7.32-7.19 (m, 1H), 7.12-6.93 (m, 1H), 5.64-5.51 (m, 1H), 4.81-4.63 (m, 2H), 4.18-4.00 (m, 2H), 3.95-3.80 (q, 1H), 3.79-3.68 (m, 1H), 3.30-3.26 (m, 1H), 3.00 (d, 2H), 2.54-2.37 (m, 1H), 2.31-2.09 (m, 2H), 2.08-1.92 (m, 1H), 1.56 (d, 3H)

Example 22, LCMS m/z=621 [M+H]+ 1H NMR (400 MHz, CD₃OD) δ ppm 8.91-8.68 (m, 1H), 7.78 (d, 1H), 7.32-7.15 (m, 1H), 7.12-6.97 (m, 1H), 5.71-5.36 (m, 1H), 4.82-4.60 (m, 2H), 4.23-3.96 (m, 2H), 3.91-3.80 (m, 1H), 3.79-3.63 (m, 1H), 3.30-3.25 (m, 1H), 3.12-2.89 (m, 2H), 2.54-2.35 (m, 1H), 2.30-2.10 (m, 2H), 2.09-1.93 (m, 1H), 1.55 (d, 3H)

### Example 23

### (R)-2-amino-4-(5-chloro-3-fluoro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]selenophene-3-carbonitrile formate or (S)-2-amino-4-(5-chloro-3-fluoro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]selenophene-3-carbonitrile formate

### And Example 24

### (S)-2-amino-4-(5-chloro-3-fluoro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]selenophene-3-carbonitrile formate and (R)-2-amino-4-(5-chloro-3-fluoro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]selenophene-3-carbonitrile formate

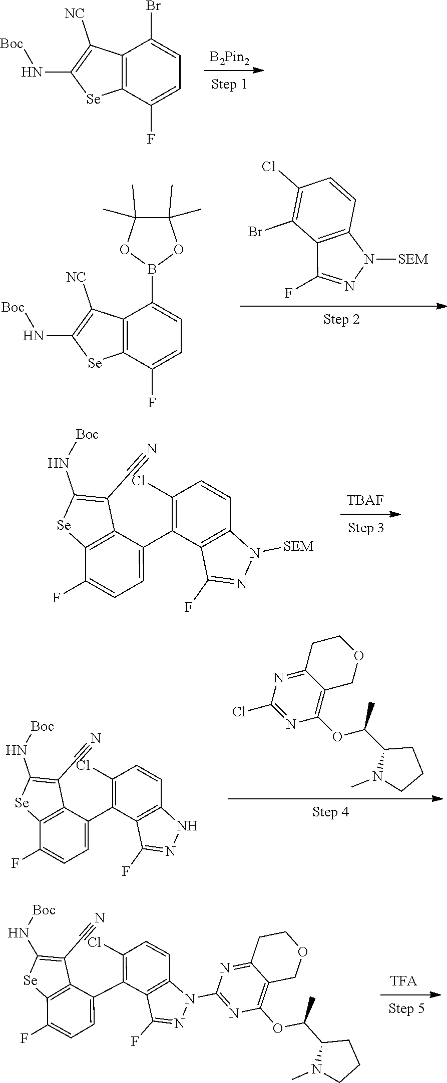

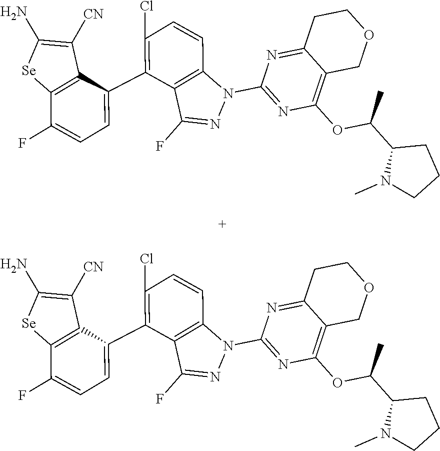

Step 1: Synthesis of tert-butyl (3-cyano-7-fluoro-4-(4,4,5,5-tetramethyl-1,3,2-dioxaborolan-2-yl)benzo[b]selenophen-2-yl)carbamate

A mixture of Intermediate 20 (1.30 g, 3.11 mmol), 4,4,5,5-tetramethyl-2-(4,4,5,5-tetramethyl-1,3,2-dioxaborolan-2-yl)-1,3,2-dioxaborolane (2.37 g, 9.33 mmol), KOAc (915 mg, 9.33 mmol) and Pd(dppf)Cl₂ (227 mg, 310 μmol) in dioxane (15 mL) was stirred at 95° C. for 2 h under N₂. The reaction mixture was diluted with water (50 mL) and extracted with EtOAc (30 mL×3). The combined organic layers were washed with brine (30 mL), dried over with anhydrous Na₂SO₄, filtered and evaporated. The residue was purified by column chromatography (SiO₂, PE/EtOAc=100/1 to 30/1) to give the title compound (620 mg, 42%) as white solid. 1H NMR (400 MHz, DMSO-d₆) δ ppm 11.89 (s, 1H), 7.70-7.47 (m, 1H), 7.28-6.96 (m, 1H), 1.53 (s, 9H), 1.35 (s, 12H)

Step 2: Synthesis of tert-butyl (4-(5-chloro-3-fluoro-1-((2-(trimethylsilyl)ethoxy)methyl)-1H-indazol-4-yl)-3-cyano-7-fluorobenzo[b]selenophen-2-yl)carbamate

The title compound was obtained as a white solid, 290 mg, 57%, from the title compound from step 1 and Intermediate 14, step 2, following a similar procedure to that described in Intermediate 14, step 3. 1H NMR (400 MHz, DMSO-d₆) δ ppm 11.76 (s, 1H), 7.99-7.79 (m, 1H), 7.67 (d, 1H), 7.43-7.36 (m, 1H), 7.35-7.24 (m, 1H), 5.83-5.50 (m, 2H), 3.58-3.48 (m, 2H), 1.50 (s, 9H), 0.86-0.78 (m, 2H), −0.01-−0.13 (m, 9H).

Step 3: Synthesis of tert-butyl (4-(5-chloro-3-fluoro-1H-indazol-4-yl)-3-cyano-7-fluorobenzo[b]selenophen-2-yl)carbamate

The title compound was obtained as a white solid (100 mg, 46%) from the title compound from step 2, following a similar procedure to that described in Intermediate 14, step 4. 1H NMR (400 MHz, DMSO-d₆) δ12.83 (s, 1H), 11.74 (s, 1H), 7.62-7.49 (m, 2H), 7.45-7.35 (m, 1H), 7.33-7.24 (m, 1H), 1.50 (s, 9H).

Step 4: Synthesis of tert-butyl (4-(5-chloro-3-fluoro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-3-cyano-7-fluorobenzo[b]selenophen-2-yl)carbamate

The title compound was obtained as a white solid, 100 mg, 66%, from the title compound from step 3 and Intermediate 1, following a similar procedure to that described in Example 3 and 4, step 1. LCMS m/z=770 [M+H]+

Step 5: Synthesis of (R)-2-amino-4-(5-chloro-3-fluoro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]selenophene-3-carbonitrile formate and (S)-2-amino-4-(5-chloro-3-fluoro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]selenophene-3-carbonitrile formate

The title compounds were obtained as Peak 1, Example 23 (26.3 mg, 37%) and Peak 2, Example 24 (35.3 mg, 5%) from the title compound of step 4, following a similar procedure to that described in Example 3 and 4, step 2.

Example 23: LCMS m/z=670 [M+H]+ 1H NMR (400 MHz, DMSO-d₆) δ ppm 8.70 (d, 1H), 8.09 (s, 2H), 7.83 (d, 1H), 7.31-7.27 (m, 1H), 7.10-7.06 (m, 1H), 5.43-5.35 (m, 1H), 4.62-4.53 (m, 2H), 4.01-3.98 (m, 2H), 3.00-2.96 (m, 1H), 2.91-2.89 (m, 1H), 2.64-2.62 (m, 1H), 2.37 (s, 3H), 2.22-2.20 (m, 1H), 1.81-1.79 (m, 1H), 1.70-1.67 (m, 3H), 1.32 (d, 3H).

Example 24:670 [M+H]+. 1H NMR (400 MHz, DMSO-d₆) δ ppm 8.70 (d, 1H), 8.10 (s, 2H), 7.85 (d, 1H), 7.31-7.27 (m, 1H), 7.10-7.06 (m, 1H), 5.42-5.37 (m, 1H), 4.66-4.57 (m, 2H), 4.03-4.00 (m, 2H), 3.40-3.10 (m, 2H) 3.00-2.90 (m, 3H), 2.74-2.67 (m, 3H), 2.30-2.10 (m, 1H), 2.20-1.70 (m, 3H), 1.42 (d, 3H).

### Example 25

### (R)-2-amino-4-(5-chloro-3-fluoro-1-(4-((S)-1-((S)-1-methylpyrrolidin-2-yl)ethoxy)-5,6,8,9-tetrahydrooxepino[4,5-d]pyrimidin-2-yl)-1H-indazol-4-yl)-7-fluorobenzo[b]thiophene-3-carbonitrile formate and (S)-2-amino-4-(5-chloro-3-fluoro-1-(4-((S)-1-((S)-1-methylpyrrolidin-2-yl)ethoxy)-5,6,8,9-tetrahydrooxepino[4,5-d]pyrimidin-2-yl)-1H-indazol-4-yl)-7-fluorobenzo[b]thiophene-3-carbonitrile formate

### And Example 26

### (S)-2-amino-4-(5-chloro-3-fluoro-1-(4-((S)-1-((S)-1-methylpyrrolidin-2-yl)ethoxy)-5,6,8,9-tetrahydrooxepino[4,5-d]pyrimidin-2-yl)-1H-indazol-4-yl)-7-fluorobenzo[b]thiophene-3-carbonitrile formate and (R)-2-amino-4-(5-chloro-3-fluoro-1-(4-((S)-1-((S)-1-methylpyrrolidin-2-yl)ethoxy)-5,6,8,9-tetrahydrooxepino[4,5-d]pyrimidin-2-yl)-1H-indazol-4-yl)-7-fluorobenzo[b]thiophene-3-carbonitrile formate

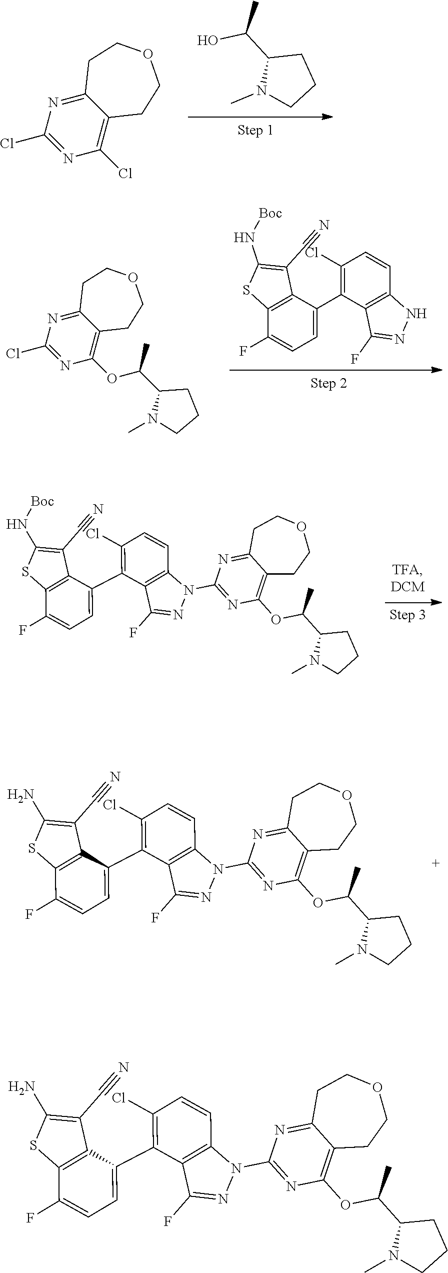

Step 1: Synthesis of 2-chloro-4-((S)-1-((S)-1-methylpyrrolidin-2-yl)ethoxy)-5,6,8,9-tetrahydrooxepino[4,5-d]pyrimidine

To(S)-1-((S)-1-methylpyrrolidin-2-yl)ethan-1-ol (323 mg, 2.50 mmol) in THF (3.0 mL) was added NaH (60% mineral oil, 109 mg, 4.56 mmol) at 0° C. and the mixture was stirred at rt for 30 min. 2,4-Dichloro-5,6,8,9-tetrahydrooxepino[4,5-d]pyrimidine (500 mg, 2.28 mmol) in THF (3.0 mL) was added at 0° C. and the reaction mixture was stirred at 25° C. for 12 h. The mixture was diluted with EtOAc (100 mL×3), the combined organic extracts were washed with water (50 mL×2), dried with Na₂SO₄ and concentrated in vacuo. The crude product was purified by column with DCM:MeOH=10:1 to give the title compound (300 mg, 42.2%) as a yellow solid. LCMS m/z=312 [M+H]+

Step 2: Synthesis of tert-butyl (4-(5-chloro-3-fluoro-1-(4-((S)-1-((S)-1-methylpyrrolidin-2-yl)ethoxy)-5,6,8,9-tetrahydrooxepino[4,5-d]pyrimidin-2-yl)-1H-indazol-4-yl)-3-cyano-7-fluorobenzo[b]thiophen-2-yl)carbamate

The title compound was obtained as a white solid, 65 mg, 52.5%, from the title compound of step 1 and Intermediate 14, following a similar procedure to that described in Example 3 and 4, step 1. LCMS m/z=736 [M+H]+

Step 3: Synthesis of (R)-2-amino-4-(5-chloro-3-fluoro-1-(4-((S)-1-((S)-1-methylpyrrolidin-2-yl)ethoxy)-5,6,8,9-tetrahydrooxepino[4,5-d]pyrimidin-2-yl)-1H-indazol-4-yl)-7-fluorobenzo[b]thiophene-3-carbonitrile formate and (S)-2-amino-4-(5-chloro-3-fluoro-1-(4-((S)-1-((S)-1-methylpyrrolidin-2-yl)ethoxy)-5,6,8,9-tetrahydrooxepino[4,5-d]pyrimidin-2-yl)-1H-indazol-4-yl)-7-fluorobenzo[b]thiophene-3-carbonitrile formate

To a solution of the title compound of step 2 (65.0 mg, 88.3 μmol) in DCM (2 mL) was added TFA (1 mL) and the mixture was stirred at 20° C. for 30 min. The reaction was concentrated. The residue was purified by Prep-HPLC (Method A3, gradient:32%-42% B over 10.0 min) to give Peak 1, Example 25 (6.80 mg, 12.1%) as white solid and Peak 2, Example 26 (19.5 mg, 34.7%) as white solid.

Example 25: LCMS m/z=635 [M+H]+ 1H NMR (400 MHz, DMSO-d₆) δ ppm 10.01-9.75 (m, 1H), 8.82-8.68 (m, 1H), 8.08 (s, 2H), 7.86 (d, 1H), 7.32 (dd, 1H), 7.16 (t, 1H), 5.49-5.27 (m, 1H), 3.91-3.79 (m, 2H), 3.78-3.65 (m, 2H), 3.65-3.43 (m, 1H), 3.33-3.13 (m, 5H), 3.09-2.78 (m, 4H), 2.34-2.19 (m, 1H), 2.15-2.03 (m, 1H), 2.00-1.71 (m, 2H), 1.60-1.21 (m, 3H).

Example 26. LCMS m/z=635 [M+H]+. 1H NMR (400 MHz, DMSO-d₆) δ ppm 9.98 (d, 1H), 8.81-8.70 (m, 1H), 8.09 (s, 2H), 7.86 (d, 1H), 7.41-7.28 (m, 1H), 7.17 (t, 1H), 5.56-5.33 (m, 1H), 3.95-3.66 (m, 5H), 3.59-3.38 (m, 1H), 3.30-3.09 (m, 3H), 3.08-2.75 (m, 5H), 2.35-2.20 (m, 1H), 2.16-1.82 (m, 3H), 1.60-1.29 (m, 3H).

### Example 27

### (S)-2-amino-4-(5-chloro-3-fluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (R)-2-amino-4-(5-chloro-3-fluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

### And Example 28

### (R)-2-amino-4-(5-chloro-3-fluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (S)-2-amino-4-(5-chloro-3-fluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

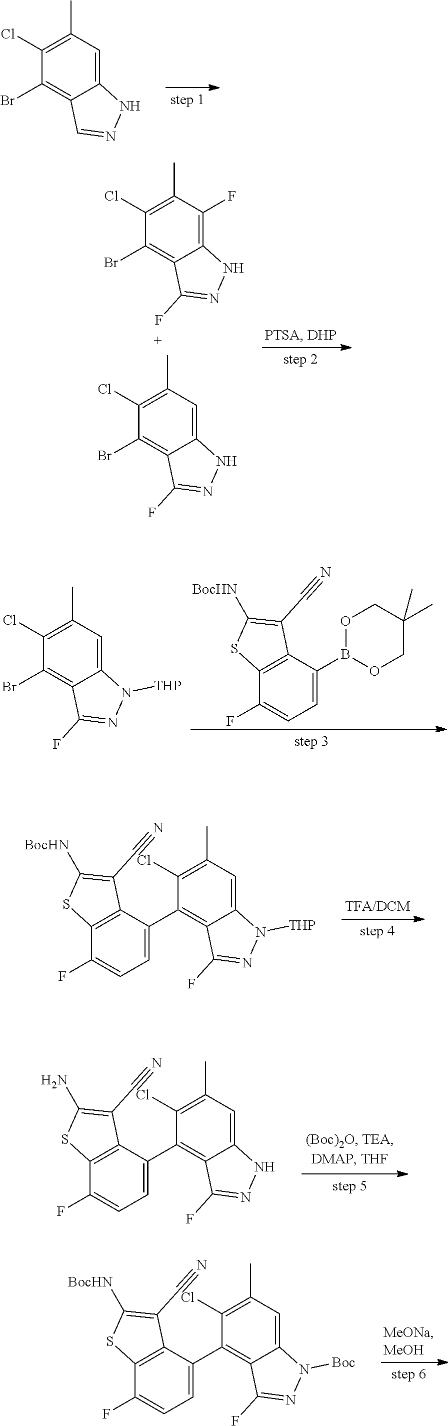

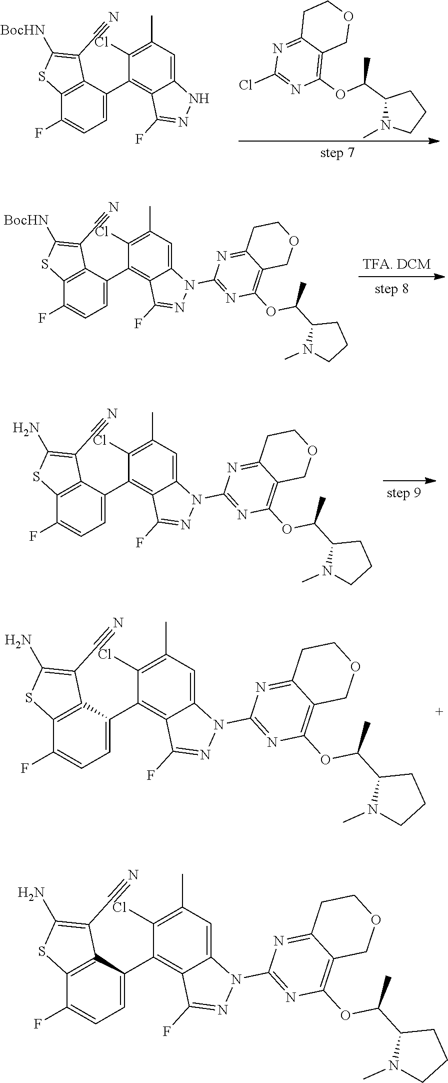

Step 1: Synthesis of 4-bromo-5-chloro-3-fluoro-6-methyl-1H-indazole

To a stirred solution of 4-bromo-5-chloro-6-methyl-1H-indazole (200 g, 814 mmol) in MeCN (2 L) was added Selectfluor (345 g, 974 mmol) and the reaction mixture was stirred for 4 h at 80° C. Water (1 L) was added to the cooled mixture and the resulting solution was extracted with DCM (3×1 L). The combined organic extracts were dried over Na₂SO₄ and concentrated in vacuo. The crude product was purified by silica gel column eluting with PE:EtOAc=10:1 to give the title compound (30 g, 14%) as a yellow solid, 4-bromo-5-chloro-7-fluoro-6-methyl-1H-indazole (35 g, 16%) and 4-bromo-5-chloro-3,7-difluoro-6-methyl-1H-indazole (20 g, 9%) LCMS: m/z=263 [M+H]+.

Step 2: Synthesis of 4-bromo-5-chloro-3-fluoro-6-methyl-1-(tetrahydro-2H-pyran-2-yl)-1H-indazole

To a stirred solution of 4-bromo-5-chloro-3-fluoro-6-methyl-1H-indazole (30 g, 113 mmol) and DHP (18.5 g, 220 mmol) in DCM (300 mL) was added TsOH (2.14 g, 11.3 mmol) and the reaction mixture was stirred for 2 h at rt. The mixture was concentrated, and the crude product was purified by silica gel column with PE:EtOAc=20:1 to give the title compound (35 g, 89%) as a white solid. ¹H NMR (400 MHz, DMSO-d₆) δ 7.84 (s, 1H), 5.89-5.63 (m, 1H), 3.90-3.79 (m, 1H), 3.76-3.54 (m, 1H), 2.51 (s, 3H), 2.29-2.10 (m, 1H), 2.06-1.80 (m, 2H), 1.76-1.61 (m, 1H), 1.59-1.48 (m, 2H).

Step 3: Synthesis of tert-butyl (4-(5-chloro-3-fluoro-6-methyl-1-(tetrahydro-2H-pyran-2-yl)-1H-indazol-4-yl)-3-cyano-7-fluorobenzo[b]thiophen-2-yl)carbamate

To a stirred solution of the title compound from step 2 (30 g, 86.3 mmol), Intermediate 9 (41.6 g, 103 mmol) and K₂CO₃ (23.7 g, 172 mmol) in dioxane (150 mL) was added Pd(dtbpf)Cl₂ (5.62 g, 8.70 mmol) and the reaction mixture was stirred for 2 h at 80° C. Water (200 mL) was added and the mixture was extracted with DCM (3×150 mL). The combined organic layer was dried over Na₂SO₄ and concentrated in vacuo. The crude product was purified by silica gel column with PE:EtOAc=3:1 to give the title compound (8.0 g, 17%) as a brown solid. LCMS m/z=559 [M+H]+

Step 4: Synthesis of 2-amino-4-(5-chloro-3-fluoro-6-methyl-1H-indazol-4-yl)-7-fluorobenzo[b]thiophene-3-carbonitrile

To a stirred solution of the title compound from step 3 (8.0 g, 14.3 mmol) in DCM (30 mL) was added TFA (10 mL) and the reaction mixture was stirred for 2 h at rt and concentrated. The mixture was adjusted to pH 7 with NH₃·MeOH solution and concentrated. The crude product was purified by silica gel column with DCM:MeOH=10:1 to give the title compound (3.8 g, 71%) as a brown solid. LCMS m/z=375 [M+H]+

Step 5: Synthesis of tert-butyl 4-(2-((tert-butoxycarbonyl)amino)-3-cyano-7-fluorobenzo[b]thiophen-4-yl)-5-chloro-3-fluoro-6-methyl-1H-indazole-1-carboxylate

To a stirred solution of the title compound from step 4 (3.8 g, 10.1 mmol), (Boc)₂O (4.40 g, 20.2 mmol) and TEA (2.04 g, 20.2 mmol) in THF (50 mL) was added DMAP (123 mg, 1.01 mmol) and the reaction mixture was stirred for 2 h at rt and concentrated. The crude product was purified by silica gel column with PE:EtOAc=10:1 to give the title compound (4.5 g, 78%) as a yellow solid. LCMS m/z=575 [M+H]+

Step 6: Synthesis of tert-butyl (4-(5-chloro-3-fluoro-6-methyl-1H-indazol-4-yl)-3-cyano-7-fluorobenzo[b]thiophen-2-yl)carbamate

To a stirred solution of the title compound from step 5 (4.5 g, 7.82 mmol) in THF (30 mL) was added NaOMe (15 mL, 30% in MeOH) and the reaction mixture was stirred for 1 h at rt and concentrated. The crude product was purified by silica gel column with PE:EtOAc=3:1 to give the title compound (3.2 g, 86%) as a yellow solid. LCMS m/z=475 [M+H]+

Step 7: Synthesis of tert-butyl (4-(5-chloro-3-fluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-3-cyano-7-fluorobenzo[b]thiophen-2-yl)carbamate

To a stirred solution of the title compound from step 6 (3.0 g, 6.31 mmol), Intermediate 1 (2.25 g, 7.57 mmol) and Cs₂CO₃ (4.09 g, 12.6 mmol) in dioxane (30 mL) were added Ephos Pd G4 (579 mg, 631 μmol) and Ephos (337 mg, 631 μmol) under N₂. The reaction mixture was stirred for 2 h at 80° C. then cooled to rt. Water (50 mL) was added and the mixture was extracted with DCM (3×50 mL). The combined organic layer was dried over Na₂SO₄ and concentrated. The crude product was purified by silica gel column with DCM:MeOH=10:1 to give the title compound (3.3 g, 71%) as a brown solid. LCMS m/z=736 [M+H]+

Step 8: Synthesis of 2-amino-4-(5-chloro-3-fluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

To a stirred solution of the title compound from step 7 (3.3 g, 4.48 mmol) in DCM (30 mL) was added TFA (10 mL) and the reaction mixture was stirred for 1 h at rt. The mixture was concentrated in vacuo and the pH of the residue adjusted to pH 7 with NH₃·MeOH solution. The solution was concentrated in vacuo and the crude product was purified by silica gel column with DCM:MeOH=10:1 (0.05% TEA) and reverse phase HPLC chromatography with MeOH:H₂O=9:1 to give the title compound (2.2 g, 77%) as a white solid. LCMS m/z=636 [M+H]+

Step 9: Synthesis of (S)-2-amino-4-(5-chloro-3-fluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile and (R)-2-amino-4-(5-chloro-3-fluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

The title compound from step 8 (2.2 g, 3.46 mmol) was purified by Prep-Chiral-SFC: Column: CHIRAL ART Cellulose-SB, 3*25 cm, 5 μm; Mobile Phase A: CO₂, Mobile Phase B: MeOH:MeCN=1:1 (0.1% 2M NH₃-MeOH); Flow rate: 100 mL/min; Gradient: isocratic 30% B, to give Peak 1, Example 27, (614.8 mg, 28%) as an off-white solid and Peak 2, Example 28, (751.3 mg, 34%) as an off-white solid.

Example 27, LCMS: m/z=636 [M+H]⁺, 1H NMR (400 MHz, DMSO-d₆) δ 8.71 (d, 1H), 8.05 (s, 2H), 7.33 (dd, 1H), 7.16 (dd, 1H), 5.40 (p, 1H), 4.65-4.51 (m, 2H), 4.00 (t, 2H), 3.02-2.87 (m, 3H), 2.63 (dt, 1H), 2.58 (s, 3H), 2.37 (s, 3H), 2.21 (td, 1H), 1.86 (ddd, 1H), 1.76-1.63 (m, 3H), 1.34 (d, 3H).

Example 28, LCMS: m/z=636 [M+H]⁺, 1H NMR (400 MHz, DMSO-d₆) δ 8.71 (d, 1H), 8.05 (s, 2H), 7.32 (dd, 1H), 7.15 (dd, 1H), 5.42 (p, 1H), 4.65-4.51 (m, 2H), 4.00 (t, 2H), 3.02-2.87 (m, 3H), 2.63 (q, 1H), 2.58 (s, 3H), 2.37 (s, 3H), 2.21 (q, 1H), 1.85 (dq, 1H), 1.78-1.62 (m, 3H), 1.34 (d, 3H).

### Example 29

### (S)-2-amino-4-(5-chloro-3-methyl-1-(4-((S)-2-methyl-1,4-diazepan-1-yl)-7,8-dihydro-5H-pyrano[4,3-d]pyrimidin-2-yl)-1H-indazol-4-yl)-7-fluorobenzo[b]thiophene-3-carbonitrile or (R)-2-amino-4-(5-chloro-3-methyl-1-(4-((S)-2-methyl-1,4-diazepan-1-yl)-7,8-dihydro-5H-pyrano[4,3-d]pyrimidin-2-yl)-1H-indazol-4-yl)-7-fluorobenzo[b]thiophene-3-carbonitrile

### And Example 30

### (R)-2-amino-4-(5-chloro-3-methyl-1-(4-((S)-2-methyl-1,4-diazepan-1-yl)-7,8-dihydro-5H-pyrano[4,3-d]pyrimidin-2-yl)-1H-indazol-4-yl)-7-fluorobenzo[b]thiophene-3-carbonitrile or (S)-2-amino-4-(5-chloro-3-methyl-1-(4-((S)-2-methyl-1,4-diazepan-1-yl)-7,8-dihydro-5H-pyrano[4,3-d]pyrimidin-2-yl)-1H-indazol-4-yl)-7-fluorobenzo[b]thiophene-3-carbonitrile

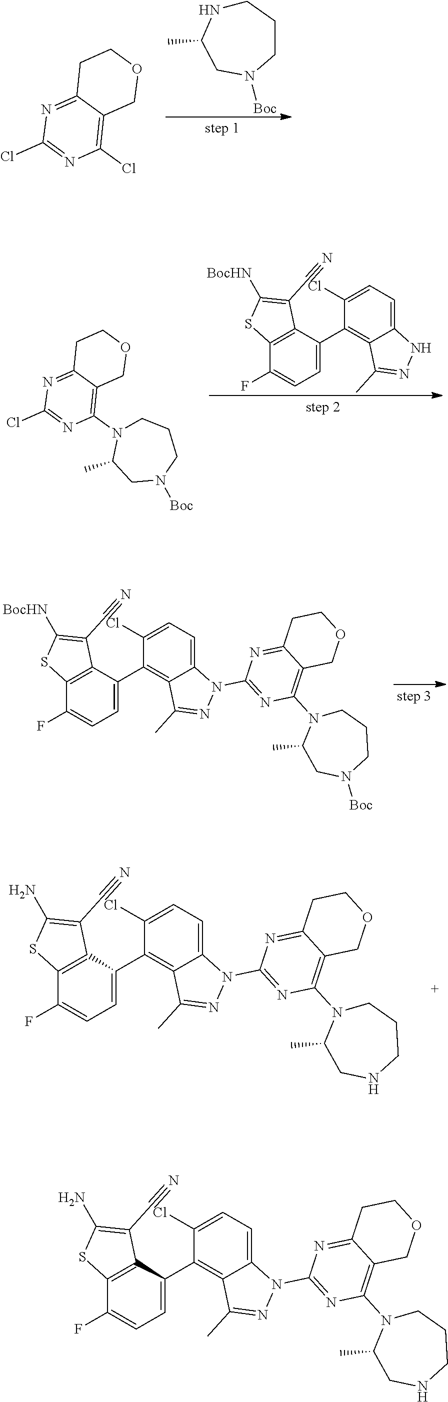

Step 1: Synthesis of tert-butyl (S)-4-(2-chloro-7,8-dihydro-5H-pyrano[4,3-d]pyrimidin-4-yl)-3-methyl-1,4-diazepane-1-carboxylate

To a stirred solution of Intermediate 1, step 1 (500 mg, 2.43 mmol) in DMF (6 mL) were added tert-butyl (S)-3-methyl-1,4-diazepane-1-carboxylate (623 mg, 2.91 mmol) and DIEA (628 mg, 4.86 mmol) and the reaction mixture was stirred overnight at 80° C. EtOAc (50 mL) was added and the resulting solution was washed with water (3×20 mL), dried over Na₂SO₄ and concentrated. The crude product was purified by silica gel column with PE:EtOAc=3:1 to give the title compound (800 mg, 86%) as a yellow oil. LCMS m/z=383 [M+H]+

Step 2: Synthesis of tert-butyl (3S)-4-(2-(4-(2-((tert-butoxycarbonyl)amino)-3-cyano-7-fluorobenzo[b]thiophen-4-yl)-5-chloro-3-methyl-1H-indazol-1-yl)-7,8-dihydro-5H-pyrano[4,3-d]pyrimidin-4-yl)-3-methyl-1,4-diazepane-1-carboxylate

To a stirred solution of the title compound from step 1 (200 mg, 522 μmol) in dioxane (5 mL) were added Intermediate 15, 286 mg, 626 μmol), Cs₂CO₃ (338 mg, 1.04 mmol), EPhos (55.6 mg, 104 μmol) and EPhos Pd G4 (47.9 mg, 52.2 μmol) under N₂. The reaction mixture was stirred for 2 h at 100° C. and then cooled to rt. Water (40 mL) was added and the resulting solution was extracted with DCM (3×40 mL). The combined organic layers were dried over Na₂SO₄ and concentrated in vacuo. The crude product was purified by Prep-TLC with DCM: MeOH=10:1 to give the title compound (120 mg, 29%) as a yellow solid. LCMS m/z=803 [M+H]+

Step 3: Synthesis of (S)-2-amino-4-(5-chloro-3-methyl-1-(4-((S)-2-methyl-1,4-diazepan-1-yl)-7,8-dihydro-5H-pyrano[4,3-d]pyrimidin-2-yl)-1H-indazol-4-yl)-7-fluorobenzo[b]thiophene-3-carbonitrile and (R)-2-amino-4-(5-chloro-3-methyl-1-(4-((S)-2-methyl-1,4-diazepan-1-yl)-7,8-dihydro-5H-pyrano[4,3-d]pyrimidin-2-yl)-1H-indazol-4-yl)-7-fluorobenzo[b]thiophene-3-carbonitrile

To a stirred solution of the title compound from step 2 (100 mg, 124 μmol) in DCM (4 mL) was added TFA (1 mL) and the reaction mixture was stirred for 2 h at rt and concentrated. Saturated NaHCO₃ solution was added to adjust the pH to 7 and the mixture was extracted with DCM (10 mL×3). The combined organic layers were dried over Na₂SO₄ and concentrated. The crude product was purified by Prep-TLC with DCM:MeOH=6:1 and the product was purified by Prep-HPLC (Method A1, Gradient: 10% B to 37% B in 15 min), to give Peak 1, Example 29 (27.8 mg, 37%) as an off-white solid and Peak 2, Example 30, (25 mg, 33%) as an off-white solid.

Example 29: LCMS: m/z=603 [M+H]⁺; 1H NMR (400 MHz, DMSO-d₆) δ 8.65 (d, 1H), 8.28 (s, 2H), 8.04 (s, 2H), 7.67 (d, 1H), 7.25 (dd, 1H), 7.16 (dd, 1H), 4.90 (d, 1H), 4.69 (d, 1H), 4.45 (s, 1H), 4.07 (dt, 1H), 3.98-3.85 (m, 2H), 3.41 (t, 2H), 3.11-3.00 (m, 1H), 2.98-2.78 (m, 4H), 1.94-1.92 (m, 1H), 1.84 (d, 1H), 1.74 (s, 3H), 1.23 (d, 3H).

Example 30: LCMS: m/z=603 [M+H]⁺; 1H NMR (400 MHz, DMSO-d₆) δ 8.64 (d, 1H), 8.27 (s, 2H), 8.04 (s, 2H), 7.66 (d, 1H), 7.24 (dd, 1H), 7.15 (dd, 1H), 4.90 (d, 1H), 4.68 (d, 1H), 4.43 (s, 1H), 4.06 (dt, 1H), 3.98-3.86 (m, 2H), 3.41 (d, 2H), 3.07-2.78 (m, 5H), 1.94-1.92 (m, 1H), 1.85 (s, 1H), 1.74 (s, 3H), 1.22 (d, 3H).

### Example 31

### (S)-2-amino-4-(5-chloro-1-(4-((S)-2,4-dimethyl-1,4-diazepan-1-yl)-7,8-dihydro-5H-pyrano[4,3-d]pyrimidin-2-yl)-3-methyl-1H-indazol-4-yl)-7-fluorobenzo[b]thiophene-3-carbonitrile or (R)-2-amino-4-(5-chloro-1-(4-((S)-2,4-dimethyl-1,4-diazepan-1-yl)-7,8-dihydro-5H-pyrano[4,3-d]pyrimidin-2-yl)-3-methyl-1H-indazol-4-yl)-7-fluorobenzo[b]thiophene-3-carbonitrile

### And Example 32

### (R)-2-amino-4-(5-chloro-1-(4-((S)-2,4-dimethyl-1,4-diazepan-1-yl)-7,8-dihydro-5H-pyrano[4,3-d]pyrimidin-2-yl)-3-methyl-1H-indazol-4-yl)-7-fluorobenzo[b]thiophene-3-carbonitrile or (S)-2-amino-4-(5-chloro-1-(4-((S)-2,4-dimethyl-1,4-diazepan-1-yl)-7,8-dihydro-5H-pyrano[4,3-d]pyrimidin-2-yl)-3-methyl-1H-indazol-4-yl)-7-fluorobenzo[b]thiophene-3-carbonitrile

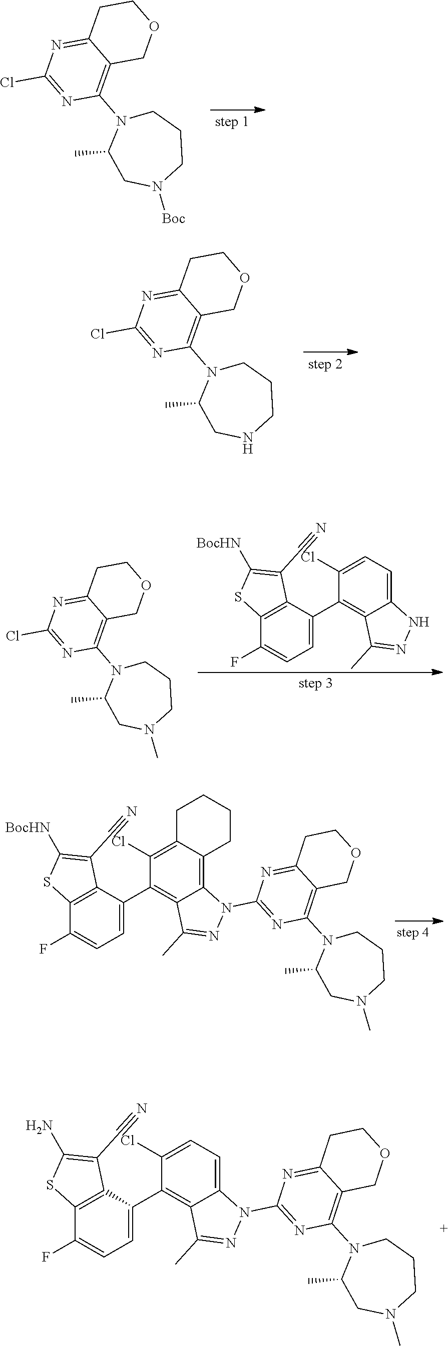

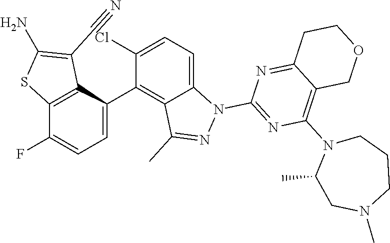

Step 1: Synthesis of (S)-2-chloro-4-(2-methyl-1,4-diazepan-1-yl)-7,8-dihydro-5H-pyrano[4,3-d]pyrimidine

The title compound was obtained as a yellow oil, 280 mg, 89%, from the compound from Example 29 and 30, step 1, following a similar procedure to that described in Example 1 and 2, step 2. LCMS m/z=283 [M+H]+

Step 2: Synthesis of (S)-2-chloro-4-(2,4-dimethyl-1,4-diazepan-1-yl)-7,8-dihydro-5H-pyrano[4,3-d]pyrimidine

To a stirred solution of the title compound from step 1 (270 mg, 954 μmol) in MeOH (5 mL) were added HCHO (387 mg, 4.77 mmol, 37% aq. solution) and sodium triacetoxyborohydride (1.0 g, 4.77 mmol) and the reaction mixture was stirred for 2 h at rt. Water (20 mL) was added and the resulting solution was extracted with DCM (3×20 mL). The combined organic layers were dried over Na₂SO₄ and concentrated in vacuo. The crude product was purified by silica gel column with DCM:MeOH=12:1 to give the title compound (150 mg, 53%) as a yellow oil. LCMS m/z=297 [M+H]+

Step 3: Synthesis of tert-butyl (4-(5-chloro-1-(4-((S)-2,4-dimethyl-1,4-diazepan-1-yl)-7,8-dihydro-5H-pyrano[4,3-d]pyrimidin-2-yl)-3-methyl-1H-indazol-4-yl)-3-cyano-7-fluorobenzo[b]thiophen-2-yl)carbamate

The title compound was obtained as a yellow solid, 200 mg, 64%, from the title compound from step 2 and Intermediate 15, following a similar procedure to that described in Example 29 and 30, step 2. LCMS m/z=717 [M+H]+

Step 4: Synthesis of (S)-2-amino-4-(5-chloro-1-(4-((S)-2,4-dimethyl-1,4-diazepan-1-yl)-7,8-dihydro-5H-pyrano[4,3-d]pyrimidin-2-yl)-3-methyl-1H-indazol-4-yl)-7-fluorobenzo[b]thiophene-3-carbonitrile and (R)-2-amino-4-(5-chloro-1-(4-((S)-2,4-dimethyl-1,4-diazepan-1-yl)-7,8-dihydro-5H-pyrano[4,3-d]pyrimidin-2-yl)-3-methyl-1H-indazol-4-yl)-7-fluorobenzo[b]thiophene-3-carbonitrile

To a stirred solution of the title compound from step 3 (180 mg, 250 μmol) in DCM (3 mL) wad added TFA (1 mL) and the reaction mixture was stirred for 2 h at rt and concentrated. Saturated aq. NaHCO₃ solution was added to adjust the pH to 7. The mixture was extracted with DCM (10 mL×3) and the combined organic layers were dried over Na₂SO₄ and concentrated. The crude product was purified by Prep-TLC with DCM:MeOH=5:1. The product was further purified by Chiral-Prep-HPLC: Column: CHIRALPAK IG, 2*25 cm, 5 μm; Mobile Phase A: Hex (0.5% 2M NH₃-MeOH), Mobile Phase B: EtOH: DCM=1:1; Flow rate: 20 mL/min; Gradient: 65% B isocratic in 6 min to give Peak 1, Example 31 (42.2 mg, 27%) as a white solid and Peak 2, Example 32 (30.1 mg, 20%) a white solid.

Example 31: LCMS: m/z=617 [M+H]⁺; 1H NMR (400 MHz, DMSO-d₆) δ 8.65 (d, 1H), 8.03 (s, 2H), 7.66 (d, 1H), 7.24 (dd, 1H), 7.15 (dd, 1H), 4.85 (d, 1H), 4.69 (d, 1H), 4.43 (q, 1H), 4.05 (dt, 1H), 3.97-3.81 (m, 2H), 3.35-3.27 (m, 1H), 3.00-2.88 (m, 2H), 2.83 (dt, 1H), 2.71-2.63 (m, 1H), 2.55 (d, 1H), 2.42 (td, 1H), 2.30 (s, 3H), 1.95 (d, 1H), 1.74 (s, 3H), 1.72-1.71 (m, 1H), 1.17 (d, 3H).

Example 32: LCMS: m/z=617 [M+H]⁺; 1H NMR (400 MHz, DMSO-d₆) δ 8.64 (d, 1H), 8.03 (s, 2H), 7.66 (d, 1H), 7.24 (dd, 1H), 7.15 (t, 1H), 4.86 (d, 1H), 4.69 (d, 1H), 4.43 (q, 1H), 4.05 (dt, 1H), 3.97-3.82 (m, 2H), 3.30 (d, 1H), 2.94 (dq, 2H), 2.88-2.79 (m, 1H), 2.71-2.63 (m, 1H), 2.59-2.51 (m, 1H), 2.43 (td, 1H), 2.30 (s, 3H), 1.95 (d, 1H), 1.74 (s, 3H), 1.72-1.71 (m, 1H), 1.18 (d, 3H).

### Example 33

### (R)-2-amino-4-(5-chloro-3-methyl-1-(4-((S)-1-((S)-1-methylpyrrolidin-2-yl)ethoxy)-6,7-dihydro-5H-cyclopenta[d]pyrimidin-2-yl)-1H-indazol-4-yl)-7-fluorobenzo[b]thiophene-3-carbonitrile or (S)-2-amino-4-(5-chloro-3-methyl-1-(4-((S)-1-((S)-1-methylpyrrolidin-2-yl)ethoxy)-6,7-dihydro-5H-cyclopenta[d]pyrimidin-2-yl)-1H-indazol-4-yl)-7-fluorobenzo[b]thiophene-3-carbonitrile

### And Example 34

### (S)-2-amino-4-(5-chloro-3-methyl-1-(4-((S)-1-((S)-1-methylpyrrolidin-2-yl)ethoxy)-6,7-dihydro-5H-cyclopenta[d]pyrimidin-2-yl)-1H-indazol-4-yl)-7-fluorobenzo[b]thiophene-3-carbonitrile or (R)-2-amino-4-(5-chloro-3-methyl-1-(4-((S)-1-((S)-1-methylpyrrolidin-2-yl)ethoxy)-6,7-dihydro-5H-cyclopenta[d]pyrimidin-2-yl)-1H-indazol -4-yl)-7-fluorobenzo[b]thiophene-3-carbonitrile

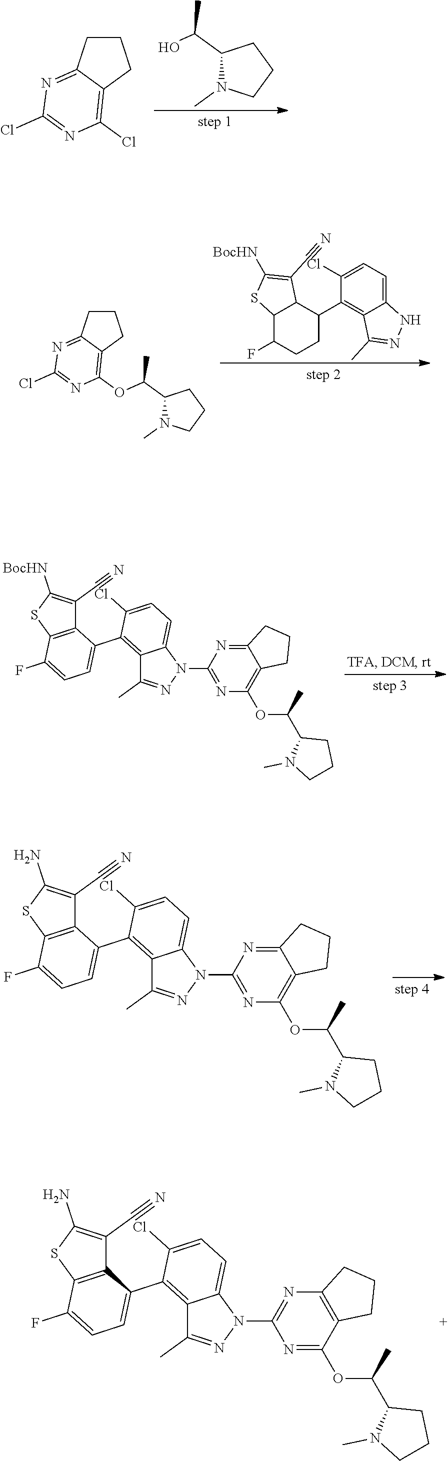

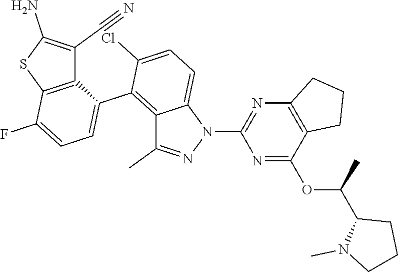

Step 1: Synthesis of 2-chloro-4-((S)-1-((S)-1-methylpyrrolidin-2-yl)ethoxy)-6,7-dihydro-5H-cyclopenta[d]pyrimidine

To a solution of (S)-1-((S)-1-methylpyrrolidin-2-yl)ethan-1-ol (92.1 mg, 713 μmol) in THF (2 mL) was added NaH (60% in mineral oil, 63.1 mg, 1.58 mmol) at 0° C. and the solution stirred for 30 min. 2,4-Dichloro-6,7-dihydro-5H-cyclopenta[d]pyrimidine (150 mg, 793 μmol) was added and the reaction mixture was stirred at rt for 2 h. The reaction was diluted with water (30 mL) and the mixture extracted with EtOAc (30 mL×3). The combined organic extracts were dried with Na₂SO₄ and concentrated in vacuo. The crude product was purified by Prep-TLC (PE:EtOAc=5:1) to give the title compound (90 mg, 40.3%) as a white solid. LCMS m/z=282 [M+H]+

Step 2: Synthesis of tert-butyl (4-(5-chloro-3-methyl-1-(4-((S)-1-((S)-1-methylpyrrolidin-2-yl)ethoxy)-6,7-dihydro-5H-cyclopenta[d]pyrimidin-2-yl)-1H-indazol-4-yl)-3-cyano-7-fluorobenzo[b]thiophen-2-yl)carbamate

The title compound was obtained as a yellow solid, 120 mg, 78.4%, from Intermediate 15 and the title compound from step 1, following a similar procedure to that described in Example 29 and 30, step 2. LCMS: m/z=702 [M+H]⁺.

Step 3: Synthesis of 2-amino-4-(5-chloro-3-methyl-1-(4-((S)-1-((S)-1-methylpyrrolidin-2-yl)ethoxy)-6,7-dihydro-5H-cyclopenta[d]pyrimidin-2-yl)-1H-indazol-4-yl)-7-fluorobenzo[b]thiophene-3-carbonitrile

To a solution of the title compound from step 2 (120 mg, 170 μmol) in DCM (1.5 mL) was added TFA (0.5 mL) and the reaction was stirred at 25° C. for 1.5 h. The resulting solution was diluted with water (30 mL) and extracted with EtOAc (30 mL×3). The combined organic layer was dried with Na₂SO₄ and concentrated in vacuo. The crude product was purified by Prep-TLC (DCM:MeOH=10:1) to give the title compound (80 mg, 78.4%) as a yellow solid. LCMS: m/z=602 [M+H]⁺.

Step 4: Synthesis of (R)-2-amino-4-(5-chloro-3-methyl-1-(4-((S)-1-((S)-1-methylpyrrolidin-2-yl)ethoxy)-6,7-dihydro-5H-cyclopenta[d]pyrimidin-2-yl)-1H-indazol-4-yl)-7-fluorobenzo[b]thiophene-3-carbonitrile and (S)-2-amino-4-(5-chloro-3-methyl-1-(4-((S)-1-((S)-1-methylpyrrolidin-2-yl)ethoxy)-6,7-dihydro-5H-cyclopenta[d]pyrimidin-2-yl)-1H-indazol-4-yl)-7-fluorobenzo[b]thiophene-3-carbonitrile

The title compound from step 3 (80 mg, 132 μmol) was separated by prep-HPLC (Method A1, Gradient: 20% B to 50% B in 10 min); to afford Peak 1, Example 33, 27.2 mg (34.2%) as an off-white solid, and Peak 2, Example 34, 30.5 mg, 38.4% as an off-white solid.

Example 33, LCMS: m/z=602 [M+H]⁺, 1H NMR (400 MHz, DMSO-d₆) δ 8.67 (d, 1H), 8.03 (s, 2H), 7.70 (d, 1H), 7.25 (dd, 1H), 7.16 (dd, 1H), 5.42 (p, 1H), 2.99 (t, 3H), 2.81 (q, 2H), 2.71 (d, 1H), 2.44 (s, 3H), 2.26 (q, 1H), 2.13 (p, 2H), 1.92-1.83 (m, 1H), 1.79-1.66 (m, 6H), 1.35 (d, 3H).

Example 34, LCMS: m/z=602 [M+H]⁺, 1H NMR (400 MHz, DMSO-d₆) δ 8.68 (d, 1H), 8.03 (s, 2H), 7.70 (d, 1H), 7.25 (dd, 1H), 7.16 (dd, 1H), 5.44 (p, 1H), 2.99 (t, 3H), 2.85-2.66 (m, 3H), 2.44 (s, 3H), 2.29-2.09 (m, 3H), 1.89-1.81 (m, 1H), 1.78-1.64 (m, 6H), 1.36 (d, 3H).

### Example 35

### (S)-2-amino-4-(5-chloro-3-methyl-1-(4-((S)-1-((S)-1-methylpyrrolidin-2-yl)ethoxy)-5,6-dihydrofuro[2,3-d]pyrimidin-2-yl)-1H-indazol-4-yl)-7-fluorobenzo[b]thiophene-3-carbonitrile or (R)-2-amino-4-(5-chloro-3-methyl-1-(4-((S)-1-((S)-1-methylpyrrolidin-2-yl)ethoxy)-5,6-dihydrofuro[2,3-d]pyrimidin-2-yl)-1H-indazol-4-yl)-7-fluorobenzo[b]thiophene-3-carbonitrile

### And Example 36

### (R)-2-amino-4-(5-chloro-3-methyl-1-(4-((S)-1-((S)-1-methylpyrrolidin-2-yl)ethoxy)-5,6-dihydrofuro[2,3-d]pyrimidin-2-yl)-1H-indazol-4-yl)-7-fluorobenzo[b]thiophene-3-carbonitrile or (S)-2-amino-4-(5-chloro-3-methyl-1-(4-((S)-1-((S)-1-methylpyrrolidin-2-yl)ethoxy)-5,6-dihydrofuro[2,3-d]pyrimidin-2-yl)-1H-indazol-4-yl)-7-fluorobenzo[b]thiophene-3-carbonitrile

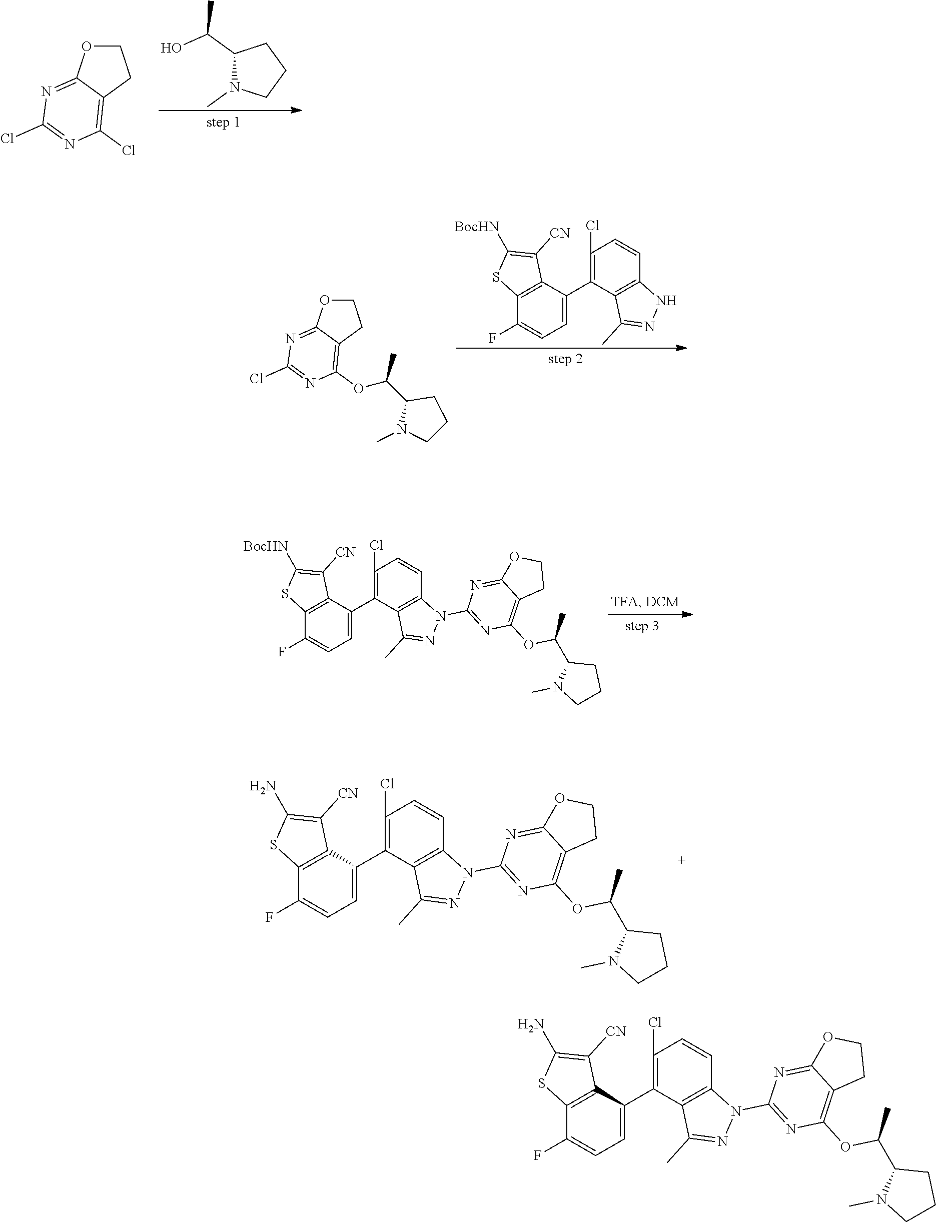

Step 1: Synthesis of 2-chloro-4-((S)-1-((S)-1-methylpyrrolidin-2-yl)ethoxy)-5,6-dihydrofuro[2,3-d]pyrimidine

The title compound was obtained as a colorless liquid, 60 mg, 20%, from 2,4-dichloro-5H,6H-furo[2,3-d]pyrimidine and (S)-1-((S)-1-methylpyrrolidin-2-yl)ethan-1-ol, following a similar procedure to that described in Intermediate 4A and B, step 2. LCMS: m/z=284 [M+H]⁺.

Step 2: Synthesis of tert-butyl (4-(5-chloro-3-methyl-1-(4-((S)-1-((S)-1-methylpyrrolidin-2-yl)ethoxy)-5,6-dihydrofuro[2,3-d]pyrimidin-2-yl)-1H-indazol-4-yl)-3-cyano-7-fluorobenzo[b]thiophen-2-yl)carbamate

The title compound was obtained as a brown solid, 100 mg, 51%, from the title compound from step 1 and Intermediate 15, following a similar procedure to that described in Example 29 and 30, step 2. LCMS m/z=704 [M+H]⁺

Step 3: Synthesis of (S)-2-amino-4-(5-chloro-3-methyl-1-(4-((S)-1-((S)-1-methylpyrrolidin-2-yl)ethoxy)-5,6-dihydrofuro[2,3-d]pyrimidin-2-yl)-1H-indazol-4-yl)-7-fluorobenzo[b]thiophene-3-carbonitrile and (R)-2-amino-4-(5-chloro-3-methyl-1-(4-((S)-1-((S)-1-methylpyrrolidin-2-yl)ethoxy)-5,6-dihydrofuro[2,3-d]pyrimidin-2-yl)-1H-indazol-4-yl)-7-fluorobenzo[b]thiophene-3-carbonitrile

To a stirred solution of the title compound from step 2 (100 mg, 142 μmol) in DCM (3 mL) was added TFA (1 mL) and the reaction mixture was stirred for 2 h at rt. The mixture was concentrated, the pH of the residue was adjusted to 7 with NH₃·MeOH solution and the solution concentrated. The crude product was purified by Prep-TLC with DCM:MeOH=10:1 and Prep-HPLC (Method A2, Gradient: 13% B to 45% B in 15 min) to give Peak 1, Example 35 (20.0 mg, 23%) as a white solid and Peak 2, Example 36, (23.2 mg, 27%) as a white solid.

Example 35. LCMS: m/z=604 [M+H]⁺, 1H NMR (400 MHz, DMSO-d₆) δ 8.62 (d, 1H), 8.04 (s, 2H), 7.71 (d, 1H), 7.31-7.10 (m, 2H), 5.40 (p, 1H), 4.85-4.58 (m, 2H), 3.13 (dp, 2H), 3.02-2.95 (m, 1H), 2.71-2.60 (m, 1H), 2.41 (s, 3H), 2.21 (q, 1H), 1.85 (dq, 1H), 1.80-1.49 (m, 6H), 1.35 (d, 3H).

Example 36. LCMS: m/z=604 [M+H]⁺, 1H NMR (400 MHz, DMSO-d₆) δ 8.63 (d, 1H), 8.05 (s, 2H), 7.71 (d, 1H), 7.25 (dd, 1H), 7.16 (dd, 1H), 5.43 (p, 1H), 4.75 (t, 2H), 3.12 (tt, 2H), 2.98-2.97 (m, 1H), 2.75-2.64 (m, 1H), 2.42 (s, 3H), 2.22 (q, 1H), 1.90-1.79 (m, 1H), 1.79-1.60 (m, 6H), 1.36 (d, 3H).

### Example 37

### 2-amino-7-fluoro-4-(1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophene-3-carbonitrile

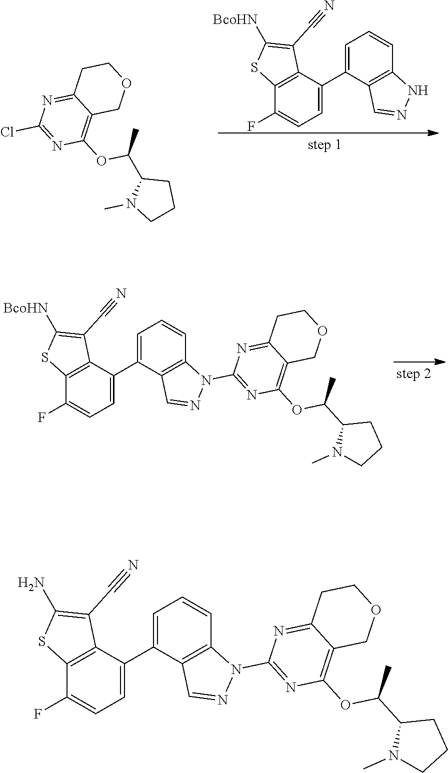

Step 1: Synthesis of tert-butyl (3-cyano-7-fluoro-4-(1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophen-2-yl)carbamate

The title compound was obtained as a brown solid, 90 mg, 81%, from Intermediate 1 and Intermediate 18, following a similar procedure to that described in Example 29 and 30, step 2. LCMS m/z=670 [M+H]+

Step 2: Synthesis of 2-amino-7-fluoro-4-(1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophene-3-carbonitrile

The title compound was obtained as a white solid, 36.4 mg, 48%, from the title compound from step 1, following a similar procedure to that described in Example 35 and 36, step 3. LCMS: m/z=570 [M+H]⁺; ¹H NMR (400 MHz, DMSO-d₆) δ 8.70 (dd, 1H), 8.01 (s, 1H), 7.97 (s, 2H), 7.71-7.58 (m, 1H), 7.42-7.25 (m, 2H), 7.14 (t, 1H), 5.48 (p, 1H), 4.71-4.49 (m, 2H), 4.01 (t, 2H), 3.02-2.87 (m, 3H), 2.65 (dt, 1H), 2.38 (s, 3H), 2.22 (q, 1H), 1.92-1.59 (m, 4H), 1.34 (d, 3H).

### Example 38

### (S)-2-amino-4-(5-chloro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (R)-2-amino-4-(5-chloro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

### And Example 39

### (R)-2-amino-4-(5-chloro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (S)-2-amino-4-(5-chloro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

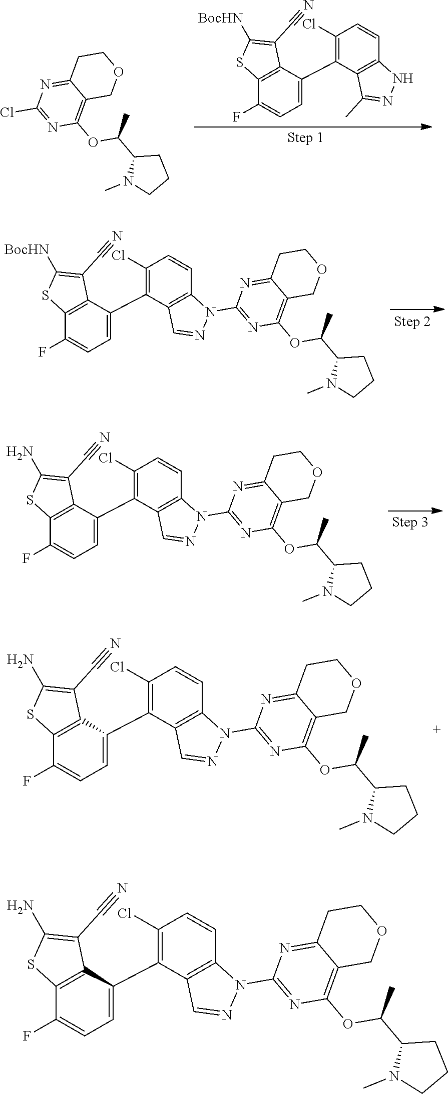

Step 1: Synthesis of tert-butyl (4-(5-chloro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-3-cyano-7-fluorobenzo[b]thiophen-2-yl)carbamate

The title compound was obtained as a yellow solid, 150 mg, 48%, from Intermediate 1 and Intermediate 16 following a similar procedure to that described in Example 29 and 30, step 2. LCMS m/z=704 [M+H]+

Step 2: Synthesis of 2-amino-4-(5-chloro-1-(4-((S)-1-((S)-pyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

To a solution of the title compound from step 1 (150 mg, 213 μmol) in DCM (2 mL) was added TFA (1 mL) and the reaction mixture was stirred for 2 h at rt and concentrated. The pH of the residue was adjusted to 7, using sat. aq. NaHCO₃ and the mixture extracted with DCM (10 mL×3). The combined organic layers were dried over Na₂SO₄ and concentrated. The crude product was purified by Prep-HPLC (Method B1, Gradient: 50% B to 80% B in 8 min), to give the title compound (100 mg, 78%) as a white solid. LCMS m/z=604 [M+H]+

Step 3: Synthesis of (S)-2-amino-4-(5-chloro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile and (R)-2-amino-4-(5-chloro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

The title compound from step 2 (100 mg, 165 μmol) was purified by Prep-Chiral-HPLC: Column: CHIRALPAK IG, 2*25 cm, 5 μm; Mobile Phase A: Hex (0.5% 2M NH₃-MeOH), Mobile Phase B: EtOH: DCM=1:1; Flow rate: 20 mL/min; Gradient: isocratic 50% B to give Peak 1, Example 38 (37.0 mg, 37%) as a white solid and Peak 2, Example 39 (40.3 mg, 40%) as a white solid.

Example 38 LCMS: m/z=604 [M+H]⁺, 1H NMR (400 MHz, DMSO-d₆) δ 8.69 (d, 1H), 8.02 (s, 2H), 7.92 (s, 1H), 7.73 (d, 1H), 7.28 (dd, 1H), 7.22-7.13 (m, 1H), 5.43 (p, 1H), 4.67-4.53 (m, 2H), 4.01 (t, 2H), 2.99-2.91 (m, 3H), 2.68 (d, 1H), 2.40 (s, 3H), 2.33-2.28 (m, 1H), 1.87-1.69 (m, 4H), 1.34 (d, 3H).

Example 39 LCMS: m/z=604 [M+H]⁺, 1H NMR (400 MHz, DMSO-d₆) δ 8.68 (d, 1H), 8.02 (s, 2H), 7.92 (s, 1H), 7.73 (d, 1H), 7.28 (dd, 1H), 7.17 (dd, 1H), 5.45 (p, 1H), 4.67-4.53 (m, 2H), 4.01 (t, 2H), 3.00-2.88 (m, 3H), 2.67-2.64 (m, 1H), 2.38 (s, 3H), 2.20 (dd, 1H), 1.90-1.70 (m, 4H), 1.34 (d, 3H).

### Example 40

### 2-amino-4-(1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophene-3-carbonitrile

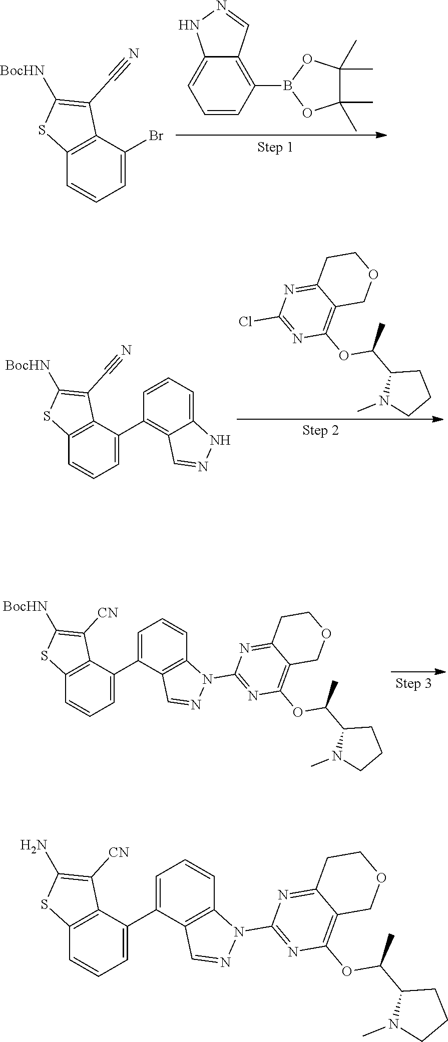 [SMILES: CC(C)(C)OC(=O)Nc1sc2cccc(Br)c2c1C#N ; CC1(C)OB(c2cccc3[nH]ncc23)OC1(C)C ; CC(C)(C)OC(=O)Nc1sc2cccc(-c3cccc4[nH]ncc34)c2c1C#N ; C[C@H](Oc1nc(Cl)nc2c1COCC2)[C@@H]1CCCN1C ; C[C@H](Oc1nc(-n2ncc3c(-c4cccc5sc(NC(=O)OC(C)(C)C)c(C#N)c45)cccc32)nc2c1COCC2)[C@@H]1CCCN1C ; C[C@H](Oc1nc(-n2ncc3c(-c4cccc5sc(N)c(C#N)c45)cccc32)nc2c1COCC2)[C@@H]1CCCN1C]

Step 1: Synthesis of tert-butyl (3-cyano-4-(1H-indazol-4-yl)benzo[b]thiophen-2-yl)carbamate

The title compound was obtained as a yellow solid, 150 mg, 67%, from tert-butyl N-(4-bromo-3-cyano-1-benzothiophen-2-yl)carbamate and 4-(4,4,5,5-tetramethyl-1,3,2-dioxaborolan-2-yl)-1H-indazole, following a similar procedure to that described in Example 27 and 28, step 3. LCMS m/z=391 [M+H]+

Step 2: Synthesis of tert-butyl (3-cyano-4-(1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophen-2-yl)carbamate

The title compound was obtained as a yellow solid, 100 mg, 46%, from tert-butyl (3-cyano-4-(1H-indazol-4-yl)benzo[b]thiophen-2-yl)carbamate and Intermediate 1, following a similar procedure to that described in Example 29 and 30, step 2. LCMS m/z=652 [M+H]+

Step 3: Synthesis of 2-amino-4-(1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophene-3-carbonitrile

The title compound was obtained as an off-white solid, 32.2 mg, 42%, from the title compound from step 2, following a similar procedure to that described in Example 38 and 39, step 2. LCMS: m/z=552 [M+H]⁺, ¹H NMR (400 MHz, DMSO-d₆) δ 8.69 (dd, 1H), 7.97 (s, 1H), 7.79 (dd, 1H), 7.71 (s, 2H), 7.63 (dd, 1H), 7.34-7.18 (m, 3H), 5.49 (dt, 1H), 4.67-4.53 (m, 2H), 4.02 (t, 2H), 2.98-2.91 (m, 3H), 2.67-2.60 (m, 1H), 2.39 (s, 3H), 2.23 (d, 1H), 1.80-1.66 (m, 4H), 1.34 (dd, 3H).

### Example 41

### 2-amino-5-fluoro-4-(1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophene-3-carbonitrile

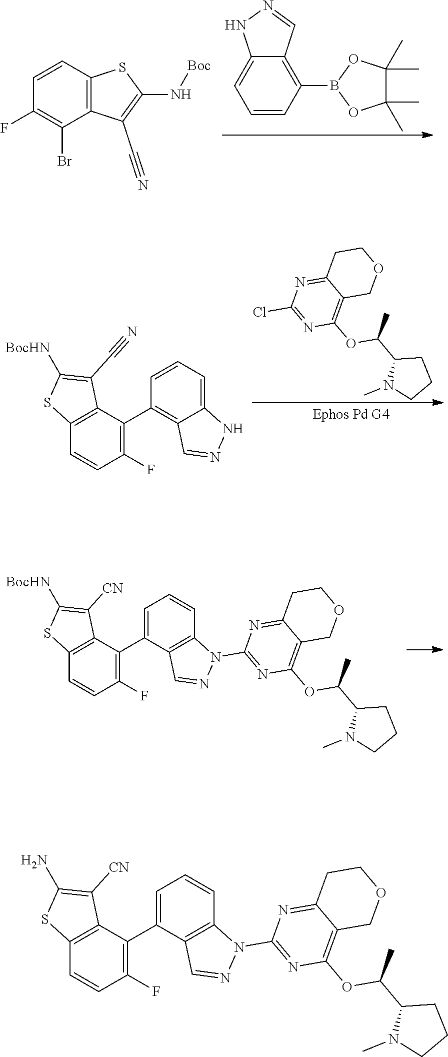 [SMILES: CC(C)(C)OC(=O)Nc1sc2ccc(F)c(Br)c2c1C#N ; CC1(C)OB(c2cccc3[nH]ncc23)OC1(C)C ; CC(C)(C)OC(=O)Nc1sc2ccc(F)c(-c3cccc4[nH]ncc34)c2c1C#N ; C[C@H](Oc1nc(Cl)nc2c1COCC2)[C@@H]1CCCN1C ; C[C@H](Oc1nc(-n2ncc3c(-c4c(F)ccc5sc(NC(=O)OC(C)(C)C)c(C#N)c45)cccc32)nc2c1COCC2)[C@@H]1CCCN1C ; C[C@H](Oc1nc(-n2ncc3c(-c4c(F)ccc5sc(N)c(C#N)c45)cccc32)nc2c1COCC2)[C@@H]1CCCN1C]

Step 1: Synthesis of tert-butyl (3-cyano-5-fluoro-4-(1H-indazol-4-yl)benzo[b]thiophen-2-yl)carbamate

The title compound was obtained as a yellow solid, 150 mg, 68%, from tert-butyl N-(4-bromo-3-cyano-5-fluoro-1-benzothiophen-2-yl)carbamate and 4-(4,4,5,5-tetramethyl-1,3,2-dioxaborolan-2-yl)-1H-indazole, following the procedure described in Example 27 and 28, step 3. LCMS: m/z=409 [M+H]⁺.

Step 2: Synthesis of tert-butyl (3-cyano-5-fluoro-4-(1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophen-2-yl)carbamate

The title compound was obtained as a yellow solid, 100 mg, 47%, from Intermediate 1 and the title compound from step 1, following a similar procedure to that described in Example 29 and 30, step 2. LCMS m/z=670 [M+H]+

Step 3: Synthesis of 2-amino-5-fluoro-4-(1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophene-3-carbonitrile

The title compound was obtained as a solid, 41.7 mg, 45%, from the title compound from step 2, following a similar procedure to that described in Example 29 and 30, step 3. LCMS: m/z=570 [M+H]⁺, 1H NMR (400 MHz, DMSO-d₆) δ 8.73 (dd, 1H), 8.00 (s, 1H), 7.86-7.78 (m, 3H), 7.65 (dd, 1H), 7.32 (d, 1H), 7.14 (t, 1H), 5.49 (dt, 1H), 4.67-4.53 (m, 2H), 4.02 (t, 2H), 3.00-2.91 (m, 3H), 2.68 (dt, 1H), 2.40 (d, 3H), 2.30-2.18 (m, 1H), 1.94-1.64 (m, 4H), 1.35 (dd, 3H).

### Example 42

### 2-amino-5,7-difluoro-4-(1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophene-3-carbonitrile

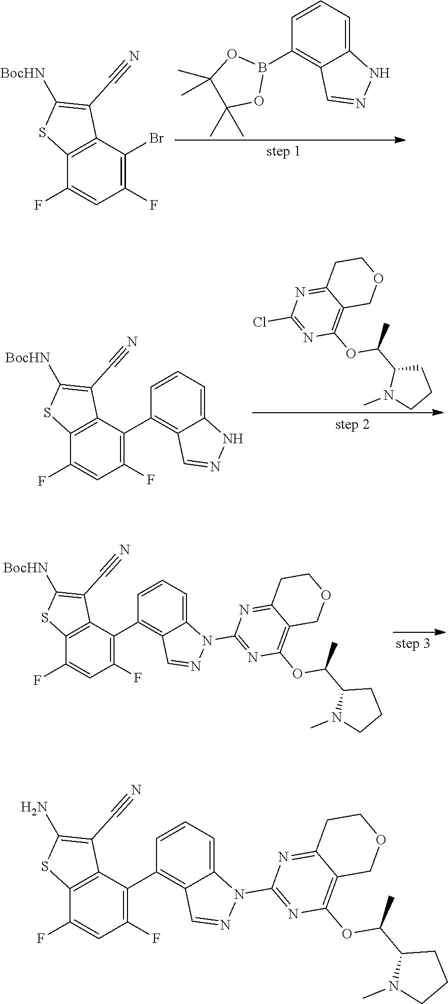 [SMILES: CC(C)(C)OC(=O)Nc1sc2c(F)cc(F)c(Br)c2c1C#N ; CC1(C)OB(c2cccc3[nH]ncc23)OC1(C)C ; CC(C)(C)OC(=O)Nc1sc2c(F)cc(F)c(-c3cccc4[nH]ncc34)c2c1C#N ; C[C@H](Oc1nc(Cl)nc2c1COCC2)[C@@H]1CCCN1C ; C[C@H](Oc1nc(-n2ncc3c(-c4c(F)cc(F)c5sc(NC(=O)OC(C)(C)C)c(C#N)c45)cccc32)nc2c1COCC2)[C@@H]1CCCN1C ; C[C@H](Oc1nc(-n2ncc3c(-c4c(F)cc(F)c5sc(N)c(C#N)c45)cccc32)nc2c1COCC2)[C@@H]1CCCN1C]

Step 1: Synthesis of tert-butyl (3-cyano-5,7-difluoro-4-(1H-indazol-4-yl)benzo[b]thiophen-2-yl)carbamate

The title compound was obtained as a brown solid, 150 mg, 91%, from Intermediate 7 and 4-(4,4,5,5-tetramethyl-1,3,2-dioxaborolan-2-yl)-1H-indazole, following a similar procedure to that described in Example 27 and 28, step 3. LCMS m/z=427 [M+H]+

Step 2: Synthesis of tert-butyl (3-cyano-5,7-difluoro-4-(1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophen-2-yl)carbamate

The title compound was obtained as a brown solid, 100 mg, 63%, from the title compound from step 1 and Intermediate 1, following a similar procedure to that described in Example 29 and 30, step 2. LCMS m/z=688 [M+H]+

Step 3: Synthesis of 2-amino-5,7-difluoro-4-(1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophene-3-carbonitrile

The title compound was obtained as a white solid, 31.5 mg, 46%, from the title compound from step 2, following a similar procedure to that described in Example 35 and 36, step 3. LCMS: m/z=588 [M+H]+; 1H NMR (400 MHz, DMSO-d₆) δ 8.74 (dd, 1H), 8.09 (s, 1H), 8.06 (s, 2H), 7.66 (dd, 1H), 7.35-7.24 (m, 2H), 5.48 (h, 1H), 4.68-4.54 (m, 2H), 4.02 (t, 2H), 3.04-2.98 (m, 1H), 2.93 (t, 2H), 2.73-2.71 (m, 1H), 2.42 (s, 3H), 2.30-2.28 (m, 1H), 1.88-1.70 (m, 4H), 1.35 (d, 3H).

### Example 43

### 2-amino-7-fluoro-4-(3-fluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophene-3-carbonitrile

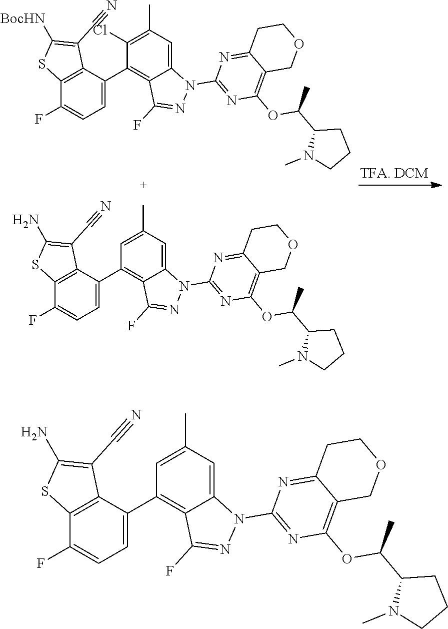 [SMILES: Cc1cc2c(c(F)nn2-c2nc3c(c(O[C@@H](C)[C@@H]4CCCN4C)n2)COCC3)c(-c2ccc(F)c3sc(NC(=O)OC(C)(C)C)c(C#N)c23)c1Cl ; Cc1cc(-c2ccc(F)c3sc(N)c(C#N)c23)c2c(F)nn(-c3nc4c(c(O[C@@H](C)[C@@H]5CCCN5C)n3)COCC4)c2c1 ; Cc1cc(-c2ccc(F)c3sc(N)c(C#N)c23)c2c(F)nn(-c3nc4c(c(O[C@@H](C)[C@@H]5CCCN5C)n3)COCC4)c2c1]

To a solution of the title compound from Example 27 and 28, step 7 (30 mg, 48.7 μmol) in DCM (0.5 mL) was added TFA (0.1 mL) and the reaction was stirred for 2 h at rt and concentrated. The mixture was adjusted pH to 7 with NH3·MeOH solution and concentrated. The crude product was purified by Prep-TLC with DCM:MeOH=5:1 and Prep-HPLC (Method B1, Gradient (B %): 65% B to 82% B in 7 min) to give the title compound (1.7 mg, yield: 4%) as a white solid. LCMS m/z=602 [M+H]⁺; 1H NMR (400 MHz, DMSO-d₆) δ 8.53 (s, 1H), 7.98 (s, 2H), 7.30 (dd, 1H), 7.18 (s, 1H), 7.12 (dd, 1H), 5.44 (h, 1H), 4.73-4.40 (m, 2H), 4.00 (t, 2H), 3.01-2.97 (m, 1H), 2.91 (t, 2H), 2.76-2.56 (m, 1H), 2.56 (s, 3H), 2.49-2.32 (m, 4H), 2.03-1.74 (m, 4H), 1.37 (d, 3H).

### Example 44

### (S)-2-amino-4-(5-chloro-3-methyl-1-(4-((S)-1-((S)-1-methylpyrrolidin-2-yl)ethoxy)-5,7-dihydrofuro[3,4-d]pyrimidin-2-yl)-1H-indazol-4-yl)-7-fluorobenzo[b]thiophene-3-carbonitrile or (R)-2-amino-4-(5-chloro-3-methyl-1-(4-((S)-1-((S)-1-methylpyrrolidin-2-yl)ethoxy)-5,7-dihydrofuro[3,4-d]pyrimidin-2-yl)-1H-indazol-4-yl)-7-fluorobenzo[b]thiophene-3-carbonitrile

### And Example 45

### (R)-2-amino-4-(5-chloro-3-methyl-1-(4-((S)-1-((S)-1-methylpyrrolidin-2-yl)ethoxy)-5,7-dihydrofuro[3,4-d]pyrimidin-2-yl)-1H-indazol-4-yl)-7-fluorobenzo[b]thiophene-3-carbonitrile or (S)-2-amino-4-(5-chloro-3-methyl-1-(4-((S)-1-((S)-1-methylpyrrolidin-2-yl)ethoxy)-5,7-dihydrofuro[3,4-d]pyrimidin-2-yl)-1H-indazol-4-yl)-7-fluorobenzo[b]thiophene-3-carbonitrile

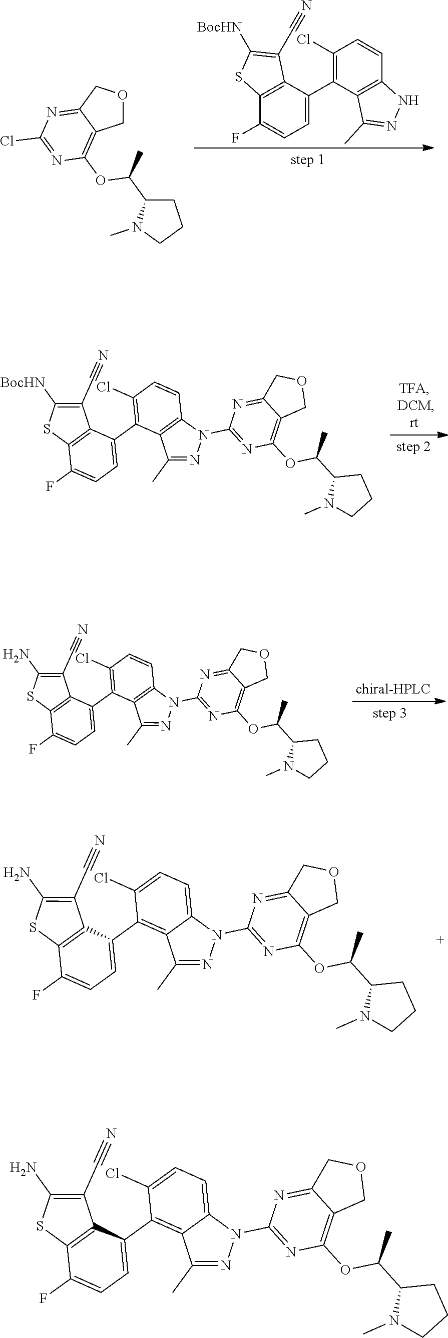

Step 1: Synthesis of tert-butyl (4-(5-chloro-3-methyl-1-(4-((S)-1-((S)-1-methylpyrrolidin-2-yl)ethoxy)-5,7-dihydrofuro[3,4-d]pyrimidin-2-yl)-1H-indazol-4-yl)-3-cyano-7-fluorobenzo[b]thiophen-2-yl)carbamate

The title compound was obtained as a yellow solid, 200 mg, 32.2%, from Intermediate 2 and Intermediate 15, following a similar procedure to that described in Example 29 and 30, step 2. LCMS m/z=704 [M+H]+

Step 2: Synthesis of 2-amino-4-(5-chloro-3-methyl-1-(4-((S)-1-((S)-1-methylpyrrolidin-2-yl)ethoxy)-5,7-dihydrofuro[3,4-d]pyrimidin-2-yl)-1H-indazol-4-yl)-7-fluorobenzo[b]thiophene-3-carbonitrile

To a solution of the title compound from step 1 (190 mg, 269 μmol) in DCM (3.0 mL) was added TFA (1.0 mL) at 0° C. and the reaction mixture was stirred at rt for 2 h. The mixture was concentrated in vacuo and the crude product was purified by HPLC (Method B1, Gradient: 56% B to 86% B in 10 min) to give the title compound, 112 mg, 68.7%, as a white solid. LCMS: m/z=604 [M+H]⁺

Step 3: Synthesis of (S)-2-amino-4-(5-chloro-3-methyl-1-(4-((S)-1-((S)-1-methylpyrrolidin-2-yl)ethoxy)-5,7-dihydrofuro[3,4-d]pyrimidin-2-yl)-1H-indazol-4-yl)-7-fluorobenzo[b]thiophene-3-carbonitrile and (R)-2-amino-4-(5-chloro-3-methyl-1-(4-((S)-1-((S)-1-methylpyrrolidin-2-yl)ethoxy)-5,7-dihydrofuro[3,4-d]pyrimidin-2-yl)-1H-indazol-4-yl)-7-fluorobenzo[b]thiophene-3-carbonitrile

The title compound from step 2 (110 mg, 182 μmol) in MeOH (4.0 mL) was purified by Chiral HPLC (Column: CHIRALPAK IG, 2*25 cm, 5 μm; Mobile Phase A: Hex (0.5% 2M NH₃-MeOH), Mobile Phase B: EtOH: DCM=1:1; Flow rate: 25 mL/min; Gradient (B %): isocratic 40, to give Peak 1, Example 44, 43.6 mg (79.4%) as a white solid and Peak 2, Example 45, 46.3 mg (84.3%) as a white solid.

Example 44: LCMS: m/z=604 [M+H]⁺, 1H NMR (400 MHz, DMSO-d₆) δ 8.65 (d, 1H), 8.05 (s, 2H), 7.72 (d, 1H), 7.27 (dd, 1H), 7.21-7.12 (m, 1H), 5.47 (p, 1H), 5.09-5.01 (m, 4H), 3.03-2.94 (m, 1H), 2.72 (s, 1H), 2.42 (s, 3H), 2.25 (q, 1H), 1.89-1.84 (m, 1H), 1.76 (s, 3H), 1.75-1.64 (m, 3H), 1.37 (d, 3H).

Example 45: LC-MS: LCMS: m/z=604 [M+H]⁺, 1H NMR (400 MHz, DMSO-d₆) δ 8.66 (d, 1H), 8.05 (s, 2H), 7.72 (d, 1H), 7.27 (dd, 1H), 7.19-7.14 (m, 1H), 5.50 (p, 1H), 5.12-4.99 (m, 4H), 3.01 (s, 1H), 2.77 (s, 1H), 2.44 (s, 3H), 2.28 (s, 1H), 1.87-1.67 (m, 7H), 1.39 (d, 3H).

### Example 46

### 2-amino-7-fluoro-4-(1-(4-((S)-1-((S)-1-methylpyrrolidin-2-yl)ethoxy)-5,7-dihydrofuro[3,4-d]pyrimidin-2-yl)-1H-indazol-4-yl)benzo[b]thiophene-3-carbonitrile

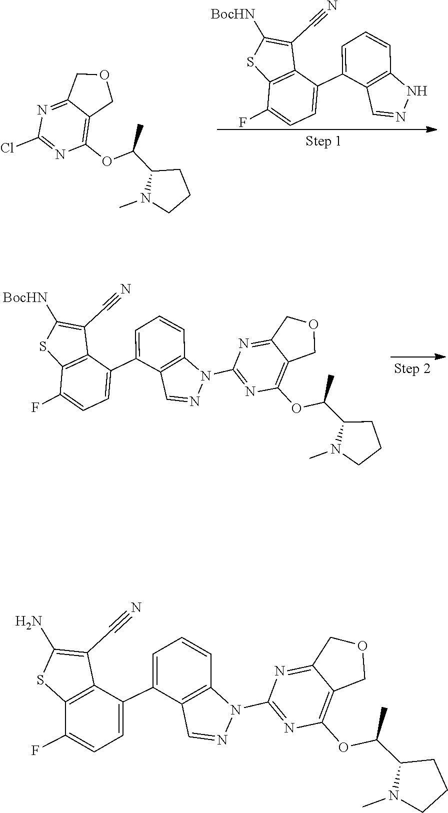 [SMILES: C[C@H](Oc1nc(Cl)nc2c1COC2)[C@@H]1CCCN1C ; CC(C)(C)OC(=O)Nc1sc2c(F)ccc(-c3cccc4[nH]ncc34)c2c1C#N ; C[C@H](Oc1nc(-n2ncc3c(-c4ccc(F)c5sc(NC(=O)OC(C)(C)C)c(C#N)c45)cccc32)nc2c1COC2)[C@@H]1CCCN1C ; C[C@H](Oc1nc(-n2ncc3c(-c4ccc(F)c5sc(N)c(C#N)c45)cccc32)nc2c1COC2)[C@@H]1CCCN1C]

Step 1: Synthesis of tert-butyl (3-cyano-7-fluoro-4-(1-(4-((S)-1-((S)-1-methylpyrrolidin-2-yl)ethoxy)-5,7-dihydrofuro[3,4-d]pyrimidin-2-yl)-1H-indazol-4-yl)benzo[b]thiophen-2-yl)carbamate

The title compound was obtained as a yellow solid, 380 mg, 65.8%, from Intermediate 2 and Intermediate 18, following a similar procedure to that described in Example 27 and 28, step 7. LCMS m/z=657 [M+H]+

Step 2: Synthesis of 2-amino-7-fluoro-4-(1-(4-((S)-1-((S)-1-methylpyrrolidin-2-yl)ethoxy)-5,7-dihydrofuro[3,4-d]pyrimidin-2-yl)-1H-indazol-4-yl)benzo[b]thiophene-3-carbonitrile

The title compound was obtained as a white solid, 190 mg, 60.7%, from the title compound from step 1, following a similar procedure to that described in Example 44 and 45, step 2. LCMS: m/z=556 [M+H]⁺, 1H NMR (400 MHz, DMSO-d₆) δ 8.68 (dd, 1H), 8.05 (s, 1H), 8.00 (s, 2H), 7.64 (dd, 1H), 7.30 (dd, 2H), 7.13 (t, 1H), 5.53 (dtd, 1H), 5.22-5.09 (m, 2H), 2.98 (d, 1H), 2.70 (s, 1H), 2.41 (s, 3H), 2.24 (d, 1H), 1.89-1.87 (m, 1H), 1.86-1.67 (m, 3H), 1.38 (dd, 3H).

### Example 47

### [(R)-2-amino-4-(5-chloro-1-((R)-6-methoxy-4-((S)-1-((S)-1-methylpyrrolidin-2-yl)ethoxy)-5,6,7,8-tetrahydroquinazolin-2-yl)-1H-indazol-4-yl)-7-fluorobenzo[b]thiophene-3-carbonitrile or (S)-2-amino-4-(5-chloro-1-((R)-6-methoxy-4-((S)-1-((S)-1-methylpyrrolidin-2-yl)ethoxy)-5,6,7,8-tetrahydroquinazolin-2-yl)-1H-indazol-4-yl)-7-fluorobenzo[b]thiophene-3-carbonitrile] or [(R)-2-amino-4-(5-chloro-1-((S)-6-methoxy-4-((S)-1-((S)-1-methylpyrrolidin-2-yl)ethoxy)-5,6,7,8-tetrahydroquinazolin-2-yl)-1H-indazol-4-yl)-7-fluorobenzo[b]thiophene-3-carbonitrile or (S)-2-amino-4-(5-chloro-1-((S)-6-methoxy-4-((S)-1-((S)-1-methylpyrrolidin-2-yl)ethoxy)-5,6,7,8-tetrahydroquinazolin-2-yl)-1H-indazol-4-yl)-7-fluorobenzo[b]thiophene-3-carbonitrile]

### And Example 48

### [(S)-2-amino-4-(5-chloro-1-((R)-6-methoxy-4-((S)-1-((S)-1-methylpyrrolidin-2-yl)ethoxy)-5,6,7,8-tetrahydroquinazolin-2-yl)-1H-indazol-4-yl)-7-fluorobenzo[b]thiophene-3-carbonitrile or (R)-2-amino-4-(5-chloro-1-((R)-6-methoxy-4-((S)-1-((S)-1-methylpyrrolidin-2-yl)ethoxy)-5,6,7,8-tetrahydroquinazolin-2-yl)-1H-indazol-4-yl)-7-fluorobenzo[b]thiophene-3-carbonitrile] or [(R)-2-amino-4-(5-chloro-1-((S)-6-methoxy-4-((S)-1-((S)-1-methylpyrrolidin-2-yl)ethoxy)-5,6,7,8-tetrahydroquinazolin-2-yl)-1H-indazol-4-yl)-7-fluorobenzo[b]thiophene-3-carbonitrile or (S)-2-amino-4-(5-chloro-1-((S)-6-methoxy-4-((S)-1-((S)-1-methylpyrrolidin-2-yl)ethoxy)-5,6,7,8-tetrahydroquinazolin-2-yl)-1H-indazol-4-yl)-7-fluorobenzo[b]thiophene-3-carbonitrile]

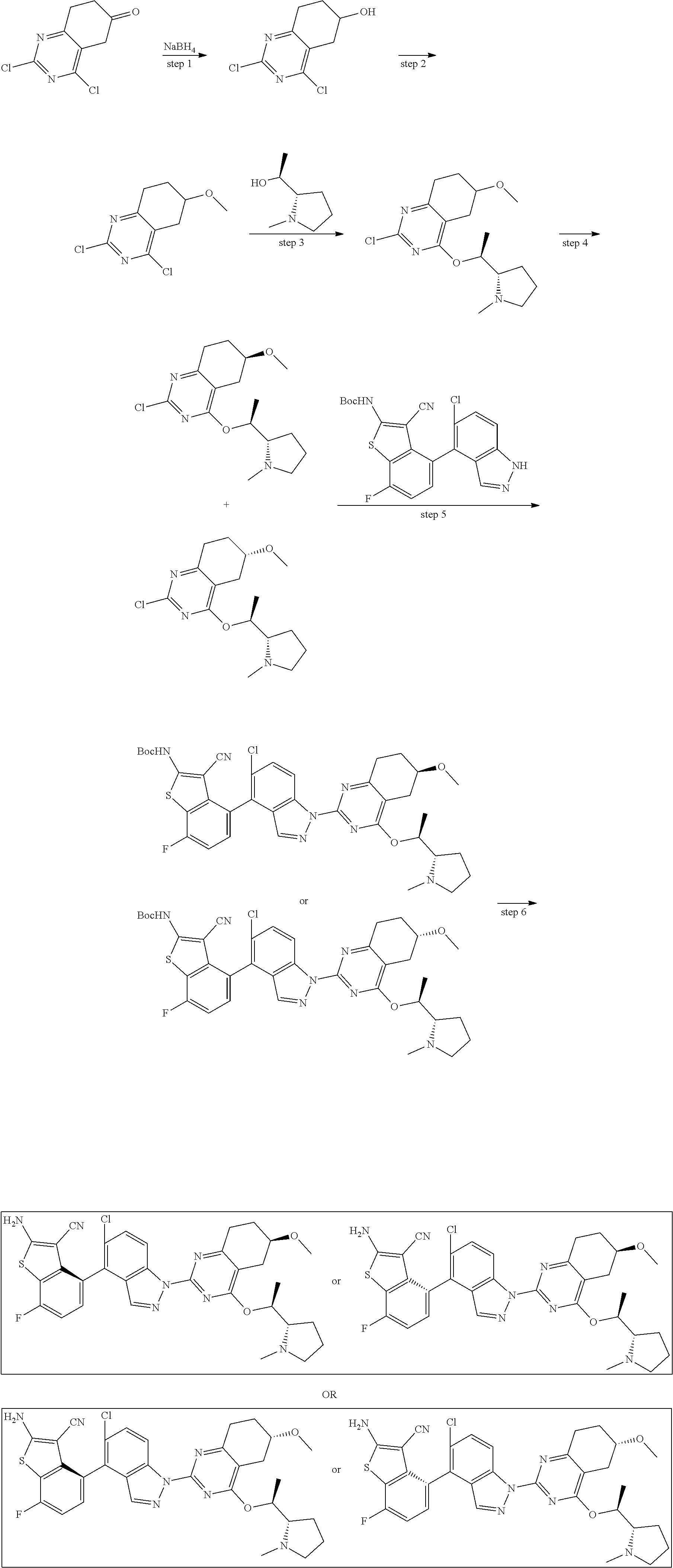

Step 1: Synthesis of 2,4-dichloro-5,6,7,8-tetrahydroquinazolin-6-ol

To a stirred solution of 2,4-dichloro-7,8-dihydroquinazolin-6 (5H)-one (1.5 g, 6.91 mmol) in EtOAc (15 mL) was added NaBH₄ (262 mg, 6.91 mmol) and the reaction was stirred for 2 h at rt. The mixture was concentrated in vacuo, the pH of the residue was adjusted to 6 using aq. HCl and filtered. The filtrate was concentrated, and the crude product was purified by silica gel column with DCM:MeOH=10:1 to give the title compound (550 mg, 55%) as a brown oil. LCMS: m/z=217 [M−H]⁻.

Step 2: Synthesis of 2,4-dichloro-6-methoxy-5,6,7,8-tetrahydroquinazoline

To a stirred solution of the title compound from step 1 (900 mg, 4.10 mmol) and Me₃O·BF₄ (1.80 g, 12.2 mmol) in DCM (10 mL) was added 1,8-bis(dimethylamino) naphthalene (2.61 g, 12.2 mmol) and the reaction was stirred for 2 h at rt. Water (20 mL) was added and the mixture was extracted with DCM (3×20 mL). The combined organic layers were dried over Na₂SO₄ and concentrated. The crude product was purified by silica gel column with PE:EtOAc=1:1 to give the title compound (300 mg, 31%) as a colorless oil. LCMS m/z=233 [M+H]+

Step 3: Synthesis of 2-chloro-6-methoxy-4-((S)-1-((S)-1-methylpyrrolidin-2-yl)ethoxy)-5,6,7,8-tetrahydroquinazoline

To a stirred solution of the title compound from step 2 (280 mg, 1.20 mmol) and (S)-1-((S)-1-methylpyrrolidin-2-yl)ethan-1-ol (170 mg, 1.32 mmol) in THF (5 mL) was added NaH (57.5 mg, 1.44 mmol) at 0° C. and the reaction mixture was stirred for 2 h at rt. Water (10 mL) was added, the resulting solution was extracted with DCM (3×10 mL) and the combined organic layers were dried over Na₂SO₄ and concentrated. The crude product was purified by Prep-TLC with DCM:MeOH=10:1 to give the title compound (100 mg, 26%) as a colorless oil. LCMS m/z=326 [M+H]+

Step 4: Synthesis of (R)-2-chloro-6-methoxy-4-((S)-1-((S)-1-methylpyrrolidin-2-yl)ethoxy)-5,6,7,8-tetrahydroquinazoline and (S)-2-chloro-6-methoxy-4-((S)-1-((S)-1-methylpyrrolidin-2-yl)ethoxy)-5,6,7,8-tetrahydroquinazoline

The title compound from step 3 (100 mg, 306 μmol) was purified by Prep-HPLC (Method A2, Gradient: 2% B to 13% B in 12 min) to give Peak 1, (R)-2-chloro-6-methoxy-4-((S)-1-((S)-1-methylpyrrolidin-2-yl)ethoxy)-5,6,7,8-tetrahydroquinazoline or (S)-2-chloro-6-methoxy-4-((S)-1-((S)-1-methylpyrrolidin-2-yl)ethoxy)-5,6,7,8-tetrahydroquinazoline (30 mg, 30%) as a colorless oil and Peak 2, (S)-2-chloro-6-methoxy-4-((S)-1-((S)-1-methylpyrrolidin-2-yl)ethoxy)-5,6,7,8-tetrahydroquinazoline or (R)-2-chloro-6-methoxy-4-((S)-1-((S)-1-methylpyrrolidin-2-yl)ethoxy)-5,6,7,8-tetrahydroquinazoline (35 mg; 35%) as a colorless oil.
Peak 1: LCMS: m/z=326 [M+H]⁺
Peak 2: LCMS: m/z=326 [M+H]⁺
   Step 5: Synthesis of tert-butyl (4-(5-chloro-1-((R)-6-methoxy-4-((S)-1-((S)-1-methylpyrrolidin-2-yl)ethoxy)-5,6,7,8-tetrahydroquinazolin-2-yl)-1H-indazol-4-yl)-3-cyano-7-fluorobenzo[b]thiophen-2-yl)carbamate or tert-butyl (4-(5-chloro-1-((S)-6-methoxy-4-((S)-1-((S)-1-methylpyrrolidin-2-yl)ethoxy)-5,6,7,8-tetrahydroquinazolin-2-yl)-1H-indazol-4-yl)-3-cyano-7-fluorobenzo[b]thiophen-2-yl)carbamate

To a stirred solution of the compound of Peak 1, step 4 (20 mg, 61.3 μmol), Intermediate 16 (32.5 mg, 73.5 μmol) and Cs₂CO₃ (39.6 mg, 122 μmol) in dioxane (2 mL) were added Ephos Pd G4 (5.62 mg, 6.13 μmol) and Ephos (3.27 mg, 6.13 μmol) under N₂. The reaction mixture was stirred for 2 h at 100° C. and cooled to rt. The crude product was purified by Prep-TLC with DCM:MeOH=10:1 to give the title compound (40 mg, 89%) as a brown solid. LCMS: m/z=732 [M+H]⁺

Step 6: Synthesis of (R)-2-amino-4-(5-chloro-1-((R)-6-methoxy-4-((S)-1-((S)-1-methylpyrrolidin-2-yl)ethoxy)-5,6,7,8-tetrahydroquinazolin-2-yl)-1H-indazol-4-yl)-7-fluorobenzo[b]thiophene-3-carbonitrile and (S)-2-amino-4-(5-chloro-1-((R)-6-methoxy-4-((S)-1-((S)-1-methylpyrrolidin-2-yl)ethoxy)-5,6,7,8-tetrahydroquinazolin-2-yl)-1H-indazol-4-yl)-7-fluorobenzo[b]thiophene-3-carbonitrile or (R)-2-amino-4-(5-chloro-1-((S)-6-methoxy-4-((S)-1-((S)-1-methylpyrrolidin-2-yl)ethoxy)-5,6,7,8-tetrahydroquinazolin-2-yl)-1H-indazol-4-yl)-7-fluorobenzo[b]thiophene-3-carbonitrile and (S)-2-amino-4-(5-chloro-1-((S)-6-methoxy-4-((S)-1-((S)-1-methylpyrrolidin-2-yl)ethoxy)-5,6,7,8-tetrahydroquinazolin-2-yl)-1H-indazol-4-yl)-7-fluorobenzo[b]thiophene-3-carbonitrile

To a stirred solution of the title compound from step 5 (30 mg, 40.9 μmol) in DCM (3 mL) was added TFA (1 mL) and the reaction mixture was stirred for 2 h at rt and concentrated. The mixture was adjusted to pH 7 with NH₃·MeOH solution and concentrated. The crude product was purified by Prep-TLC with DCM:MeOH=5:1 and further purified by Prep-HPLC (Method A2, Gradient: 15% B to 45% B in 15 min) to give Peak 1, Example 47 (3.8 mg, 15%) as a white solid and Peak 2, Example 48, (6.2 mg, 24%) as a white solid.

Example 47, LCMS: m/z=632 [M+H]⁺, 1H NMR (400 MHz, DMSO-d₆) δ 8.67 (dd, 1H), 8.02 (s, 2H), 7.89 (d, 1H), 7.71 (d, 1H), 7.28 (dd, 1H), 7.17 (dd, 1H), 5.41 (p, 1H), 3.78 (t, 1H), 3.30 (s, 3H), 3.06-2.83 (m, 3H), 2.81 (dd, 1H), 2.75 (d, 1H), 2.60 (dd, 1H), 2.40 (s, 3H), 2.24-2.23 (m, 1H), 2.00 (d, 2H), 1.87 (d, 1H), 1.83-1.62 (m, 3H), 1.34 (d, 3H).

Example 48, LCMS: m/z=632 [M+H]⁺, 1H NMR (400 MHz, DMSO-d₆) δ 8.68 (dd, 1H), 8.02 (s, 2H), 7.89 (d, 1H), 7.71 (d, 1H), 7.28 (dd, 1H), 7.17 (dd, 1H), 5.42 (p, 1H), 3.77 (q, 1H), 3.30 (s, 3H), 3.05-2.84 (m, 3H), 2.81 (dd, 1H), 2.66-2.5 (m, 2H), 2.39 (s, 3H), 2.22-2.21 (m, 1H), 2.01 (t, 2H), 1.85 (d, 1H), 1.76-1.68 (m, 3H), 1.34 (d, 3H).

### Example 49

### [(R)-2-amino-4-(5-chloro-1-((S)-6-methoxy-4-((S)-1-((S)-1-methylpyrrolidin-2-yl)ethoxy)-5,6,7,8-tetrahydroquinazolin-2-yl)-1H-indazol-4-yl)-7-fluorobenzo[b]thiophene-3-carbonitrile or (S)-2-amino-4-(5-chloro-1-((S)-6-methoxy-4-((S)-1-((S)-1-methylpyrrolidin-2-yl)ethoxy)-5,6,7,8-tetrahydroquinazolin-2-yl)-1H-indazol-4-yl)-7-fluorobenzo[b]thiophene-3-carbonitrile] or [(R)-2-amino-4-(5-chloro-1-((R)-6-methoxy-4-((S)-1-((S)-1-methylpyrrolidin-2-yl)ethoxy)-5,6,7,8-tetrahydroquinazolin-2-yl)-1H-indazol-4-yl)-7-fluorobenzo[b]thiophene-3-carbonitrile or (S)-2-amino-4-(5-chloro-1-((R)-6-methoxy-4-((S)-1-((S)-1-methylpyrrolidin-2-yl)ethoxy)-5,6,7,8-tetrahydroquinazolin-2-yl)-1H-indazol-4-yl)-7-fluorobenzo[b]thiophene-3-carbonitrile]

### And Example 50

### [(S)-2-amino-4-(5-chloro-1-((S)-6-methoxy-4-((S)-1-((S)-1-methylpyrrolidin-2-yl)ethoxy)-5,6,7,8-tetrahydroquinazolin-2-yl)-1H-indazol-4-yl)-7-fluorobenzo[b]thiophene-3-carbonitrile or (R)-2-amino-4-(5-chloro-1-((S)-6-methoxy-4-((S)-1-((S)-1-methylpyrrolidin-2-yl)ethoxy)-5,6,7,8-tetrahydroquinazolin-2-yl)-1H-indazol-4-yl)-7-fluorobenzo[b]thiophene-3-carbonitrile] or [(S)-2-amino-4-(5-chloro-1-((R)-6-methoxy-4-((S)-1-((S)-1-methylpyrrolidin-2-yl)ethoxy)-5,6,7,8-tetrahydroquinazolin-2-yl)-1H-indazol-4-yl)-7-fluorobenzo[b]thiophene-3-carbonitrile or (R)-2-amino-4-(5-chloro-1-((R)-6-methoxy-4-((S)-1-((S)-1-methylpyrrolidin-2-yl)ethoxy)-5,6,7,8-tetrahydroquinazolin-2-yl)-1H-indazol-4-yl)-7-fluorobenzo[b]thiophene-3-carbonitrile]

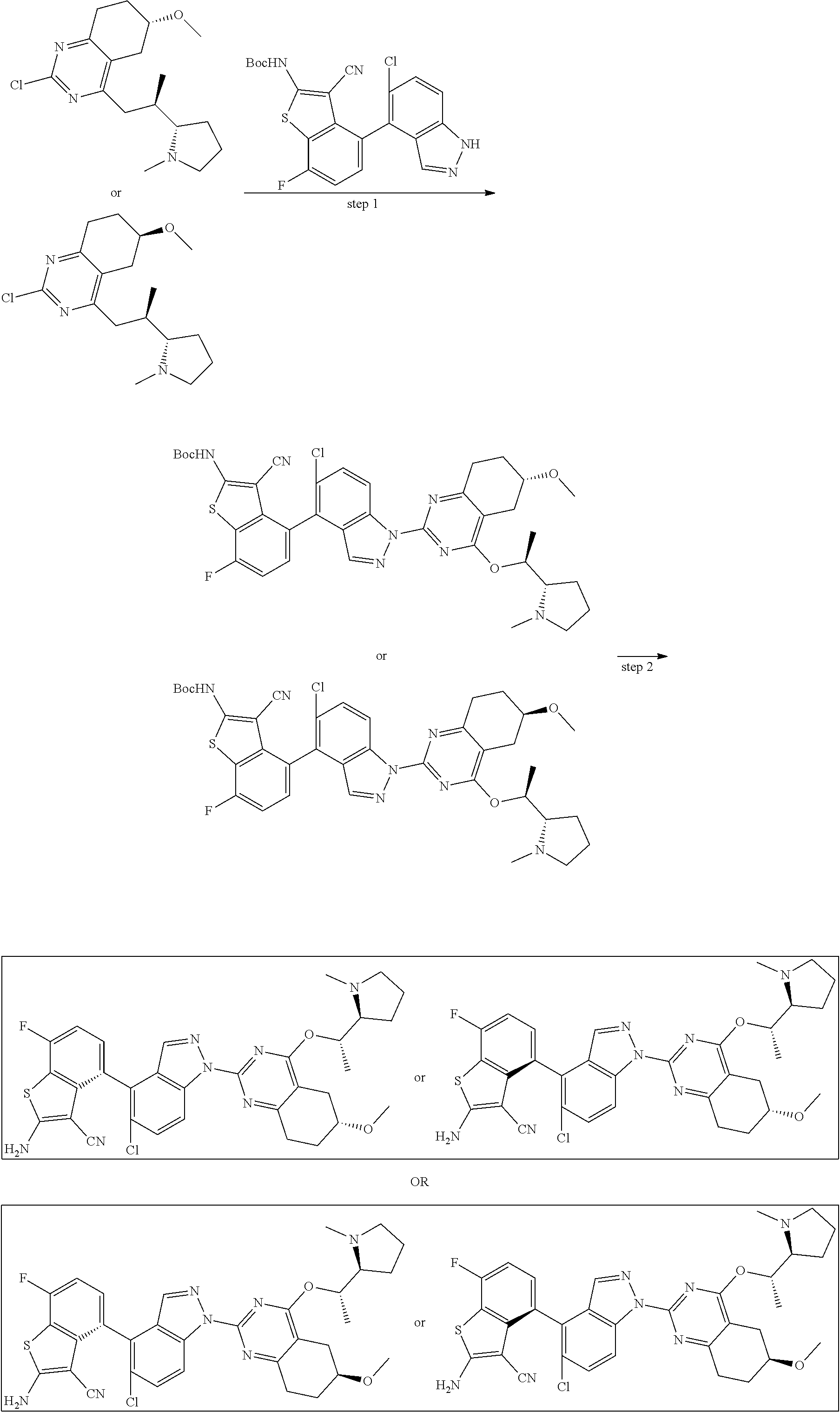

Step 1: Synthesis of tert-butyl (4-(5-chloro-1-((S)-6-methoxy-4-((S)-1-((S)-1-methylpyrrolidin-2-yl)ethoxy)-5,6,7,8-tetrahydroquinazolin-2-yl)-1H-indazol-4-yl)-3-cyano-7-fluorobenzo[b]thiophen-2-yl)carbamate or tert-butyl (4-(5-chloro-1-((R)-6-methoxy-4-((S)-1-((S)-1-methylpyrrolidin-2-yl)ethoxy)-5,6,7,8-tetrahydroquinazolin-2-yl)-1H-indazol-4-yl)-3-cyano-7-fluorobenzo[b]thiophen-2-yl)carbamate

The title compound was obtained as a brown solid, 50 mg, 89%, from the product of Example 47 and 48, step 4, peak 2 and Intermediate 16, following the procedure described in Example 47 and 48, step 5. LCMS m/z=732 [M+H]+

Step 2: Synthesis of (R)-2-amino-4-(5-chloro-1-((S)-6-methoxy-4-((S)-1-((S)-1-methylpyrrolidin-2-yl)ethoxy)-5,6,7,8-tetrahydroquinazolin-2-yl)-1H-indazol-4-yl)-7-fluorobenzo[b]thiophene-3-carbonitrile and (S)-2-amino-4-(5-chloro-1-((S)-6-methoxy-4-((S)-1-((S)-1-methylpyrrolidin-2-yl)ethoxy)-5,6,7,8-tetrahydroquinazolin-2-yl)-1H-indazol-4-yl)-7-fluorobenzo[b]thiophene-3-carbonitrile or (R)-2-amino-4-(5-chloro-1-((R)-6-methoxy-4-((S)-1-((S)-1-methylpyrrolidin-2-yl)ethoxy)-5,6,7,8-tetrahydroquinazolin-2-yl)-1H-indazol-4-yl)-7-fluorobenzo[b]thiophene-3-carbonitrile and (S)-2-amino-4-(5-chloro-1-((R)-6-methoxy-4-((S)-1-((S)-1-methylpyrrolidin-2-yl)ethoxy)-5,6,7,8-tetrahydroquinazolin-2-yl)-1H-indazol-4-yl)-7-fluorobenzo[b]thiophene-3-carbonitrile

The title compounds were obtained from the title compound from step 1, following a similar procedure to that described in Example 47 and 48, step 6.

Peak 1, Example 49 (10.8 mg, 25%) as a white solid. LCMS: m/z=632 [M+H]⁺, 1H NMR (400 MHz, DMSO-d₆) δ 8.68 (dd, 1H), 8.02 (s, 2H), 7.89 (d, 1H), 7.71 (d, 1H), 7.28 (dd, 1H), 7.17 (dd, 1H), 5.41 (p, 1H), 3.77 (dq, 1H), 3.32 (s, 3H), 2.98-2.64 (m, 5H), 2.67-2.57 (m, 1H), 2.39 (s, 3H), 2.23-2.11 (m, 1H), 2.11-1.96 (m, 2H), 1.87-1.68 (m, 4H), 1.33 (d, 3H).

and Peak 2, Example 50 (11.6 mg, 26%) as a white solid. LCMS: m/z=632 [M+H]⁺, 1H NMR (400 MHz, DMSO-d₆) δ 8.68 (d, 1H), 8.03 (s, 2H), 7.89 (s, 1H), 7.71 (d, 1H), 7.28 (dd, 1H), 7.17 (dd, 1H), 5.43 (p, 1H), 3.77-3.72 (m, 1H), 3.32 (s, 3H), 2.98 (dq, 1H), 2.93-2.67 (m, 4H), 2.58 (d, 1H), 2.40 (s, 3H), 2.24 (q, 1H), 2.00 (dt, 2H), 1.87-1.70 (m, 4H), 1.34 (d, 3H).

### Example 51

### [2-amino-4-((S)-5-chloro-1-((R)-8-methyl-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or 2-amino-4-((R)-5-chloro-1-((R)-8-methyl-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile] or [2-amino-4-((S)-5-chloro-1-((S)-8-methyl-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or 2-amino-4-((R)-5-chloro-1-((S)-8-methyl-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile]

### And Example 52

### [2-amino-4-((R)-5-chloro-1-((R)-8-methyl-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or 2-amino-4-((S)-5-chloro-1-((R)-8-methyl-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile] or [2-amino-4-((R)-5-chloro-1-((S)-8-methyl-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or 2-amino-4-((S)-5-chloro-1-((S)-8-methyl-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile]

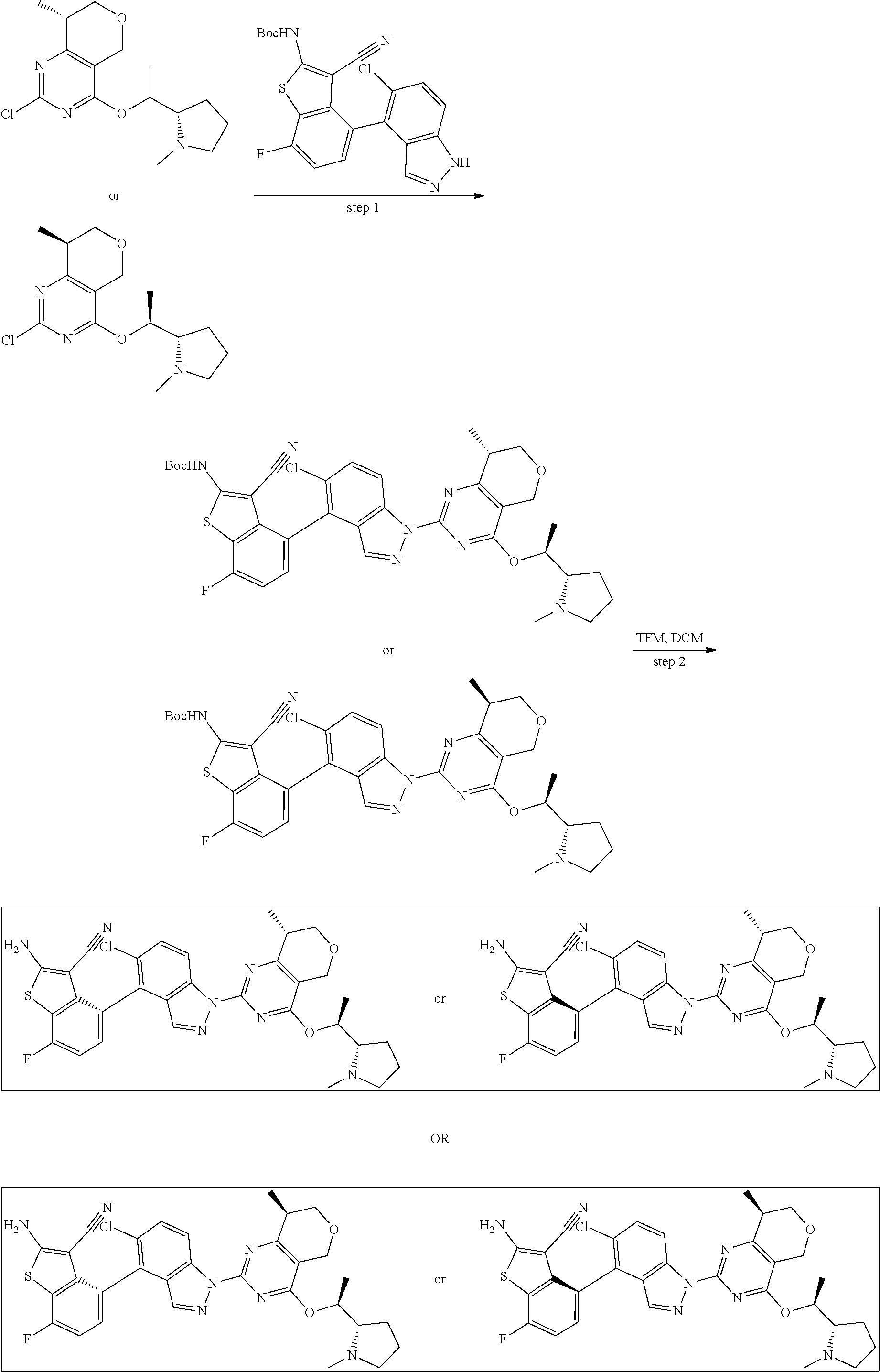

Step 1: Synthesis of tert-butyl (4-(5-chloro-1-((S)-8-methyl-4-((S)-1-((S)-1-methylpyrrolidin-2-yl)ethoxy)-7,8-dihydro-5H-pyrano[4,3-d]pyrimidin-2-yl)-1H-indazol-4-yl)-3-cyano-7-fluorobenzo[b]thiophen-2-yl)carbamate or tert-butyl (4-(5-chloro-1-((R)-8-methyl-4-((S)-1-((S)-1-methylpyrrolidin-2-yl)ethoxy)-7,8-dihydro-5H-pyrano[4,3-d]pyrimidin-2-yl)-1H-indazol-4-yl)-3-cyano-7-fluorobenzo[b]thiophen-2-yl)carbamate

The title compound was obtained as a yellow solid, 70 mg, 30%, from Intermediate 5B and Intermediate 16, following a similar procedure to that described in Example 29 and 30, step 2. LCMS m/z=718 [M+H]+

Step 2: Synthesis of [2-amino-4-((S)-5-chloro-1-((R)-8-methyl-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile and 2-amino-4-((R)-5-chloro-1-((R)-8-methyl-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile] or [2-amino-4-((S)-5-chloro-1-((S)-8-methyl-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile and 2-amino-4-((R)-5-chloro-1-((S)-8-methyl-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile]

The title compounds were obtained from the title compound from step 1, following a similar procedure to that described in Example 1 and 2, steps 2 and 3.

Peak 1, Example 51, (12.8 mg, 37%) as a white solid. LCMS: m/z=618 [M+H]⁺1H NMR (400 MHz, DMSO-d₆) δ 8.69 (d, 1H), 8.04 (s, 2H), 7.93 (s, 1H), 7.75 (d, 1H), 7.28 (dd, 1H), 7.18 (t, 1H), 5.48-5.39 (m, 1H), 4.69-4.56 (m, 2H), 4.06 (dd, 1H), 3.69 (dd, 1H), 3.06-3.01 (m, 2H), 2.79-2.74 (m, 1H), 2.44 (s, 3H), 2.36-2.31 (m, 1H), 1.91-1.87 (m, 1H), 1.81-1.65 (m, 3H), 1.36 (d, 3H), 1.33 (d, 3H).

Peak 2 Example 52 (10.8 mg, 31%) as a white solid. LCMS: m/z=618 [M+H]⁺1H NMR (400 MHz, DMSO-d₆) δ 8.70 (d, 1H), 8.04 (s, 2H), 7.93 (s, 1H), 7.76 (d, 1H), 7.28 (dd, 1H), 7.20-7.15 (m, 1H), 5.43 (t, 1H), 4.63-4.59 (m, 2H), 4.06 (dd, 1H), 3.69 (dd, 1H), 3.12-2.98 (m, 2H), 2.68-2.65 (m, 1H), 2.37 (s, 3H), 2.34-2.31 (m, 1H), 1.87-1.824 (m, 1H), 1.74-1.65 (m, 3H), 1.37 (d, 3H), 1.33 (d, 3H).

### Example 53

### [(S)-2-amino-4-(5-chloro-1-((5S,8R)-4-((S)-1-((S)-1-methylpyrrolidin-2-yl)ethoxy)-6,7,8,9-tetrahydro-5H-5,8-epoxycyclohepta[d]pyrimidin-2-yl)-1H-indazol-4-yl)-7-fluorobenzo[b]thiophene-3-carbonitrile or (R)-2-amino-4-(5-chloro-1-((5S,8R)-4-((S)-1-((S)-1-methylpyrrolidin-2-yl)ethoxy)-6,7,8,9-tetrahydro-5H-5,8-epoxycyclohepta[d]pyrimidin-2-yl)-1H-indazol-4-yl)-7-fluorobenzo[b]thiophene-3-carbonitrile] or [(S)-2-amino-4-(5-chloro-1-((5R,8S)-4-((S)-1-((S)-1-methylpyrrolidin-2-yl)ethoxy)-6,7,8,9-tetrahydro-5H-5,8-epoxycyclohepta[d]pyrimidin-2-yl)-1H-indazol-4-yl)-7-fluorobenzo[b]thiophene-3-carbonitrile or (R)-2-amino-4-(5-chloro-1-((5R,8S)-4-((S)-1-((S)-1-methylpyrrolidin-2-yl)ethoxy)-6,7,8,9-tetrahydro-5H-5,8-epoxycyclohepta[d]pyrimidin-2-yl)-1H-indazol-4-yl)-7-fluorobenzo[b]thiophene-3-carbonitrile]

### And Example 54

### [(R)-2-amino-4-(5-chloro-1-((5S,8R)-4-((S)-1-((S)-1-methylpyrrolidin-2-yl)ethoxy)-6,7,8,9-tetrahydro-5H-5,8-epoxycyclohepta[d]pyrimidin-2-yl)-1H-indazol-4-yl)-7-fluorobenzo[b]thiophene-3-carbonitrile or (S)-2-amino-4-(5-chloro-1-((5S,8R)-4-((S)-1-((S)-1-methylpyrrolidin-2-yl)ethoxy)-6,7,8,9-tetrahydro-5H-5,8-epoxycyclohepta[d]pyrimidin-2-yl)-1H-indazol-4-yl)-7-fluorobenzo[b]thiophene-3-carbonitrile] or [(R)-2-amino-4-(5-chloro-1-((5R,8S)-4-((S)-1-((S)-1-methylpyrrolidin-2-yl)ethoxy)-6,7,8,9-tetrahydro-5H-5,8-epoxycyclohepta[d]pyrimidin-2-yl)-1H-indazol-4-yl)-7-fluorobenzo[b]thiophene-3-carbonitrile or (S)-2-amino-4-(5-chloro-1-((5R,8S)-4-((S)-1-epoxycyclohepta[d]pyrimidin-2-yl)-1H-indazol-4-yl)-7-fluorobenzo[b]thiophene-3-carbonitrile]

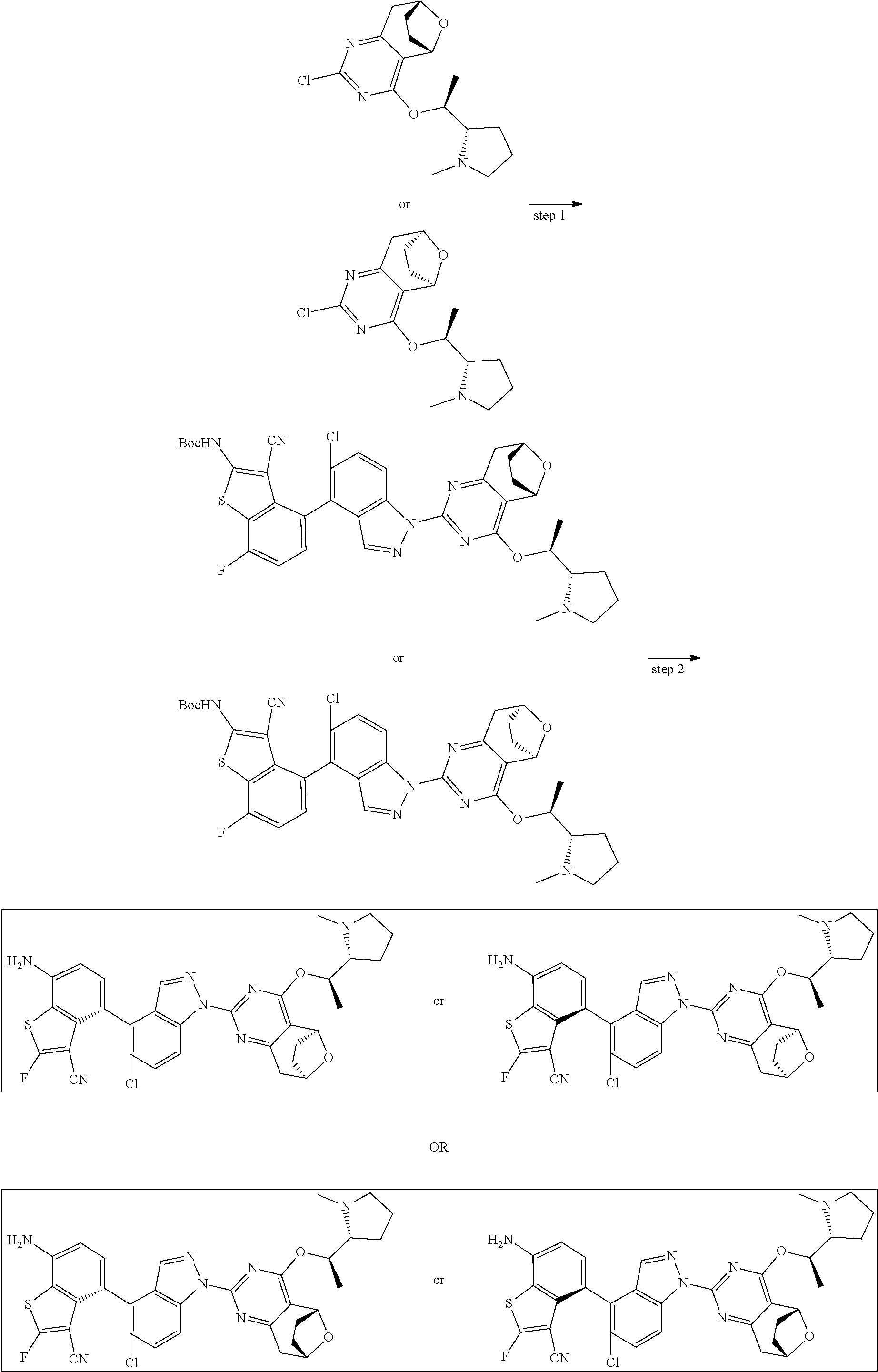

Step 1: Synthesis of tert-butyl (4-(5-chloro-1-((5S,8R)-4-((S)-1-((S)-1-methylpyrrolidin-2-yl)ethoxy)-6,7,8,9-tetrahydro-5H-5,8-epoxycyclohepta[d]pyrimidin-2-yl)-1H-indazol-4-yl)-3-cyano-7-fluorobenzo[b]thiophen-2-yl)carbamate or tert-butyl (4-(5-chloro-1-((5R,8S)-4-((S)-1-((S)-1-methylpyrrolidin-2-yl)ethoxy)-6,7,8,9-tetrahydro-5H-5,8-epoxycyclohepta[d]pyrimidin-2-yl)-1H-indazol-4-yl)-3-cyano-7-fluorobenzo[b]thiophen-2-yl)carbamate

The title compound was obtained as a yellow solid, 60 mg, 60%, from Intermediate 3 A and Intermediate 16, following a similar procedure to that described in Example 29 and 30, step 2.

Step 2: Synthesis of (S)-2-amino-4-(5-chloro-1-((5S,8R)-4-((S)-1-((S)-1-methylpyrrolidin-2-yl)ethoxy)-6,7,8,9-tetrahydro-5H-5,8-epoxycyclohepta[d]pyrimidin-2-yl)-1H-indazol-4-yl)-7-fluorobenzo[b]thiophene-3-carbonitrile and (R)-2-amino-4-(5-chloro-1-((5S,8R)-4-((S)-1-((S)-1-methylpyrrolidin-2-yl)ethoxy)-6,7,8,9-tetrahydro-5H-5,8-epoxycyclohepta[d]pyrimidin-2-yl)-1H-indazol-4-yl)-7-fluorobenzo[b]thiophene-3-carbonitrile or (S)-2-amino-4-(5-chloro-1-((5R,8S)-4-((S)-1-((S)-1-methylpyrrolidin-2-yl)ethoxy)-6,7,8,9-tetrahydro-5H-5,8-epoxycyclohepta[d]pyrimidin-2-yl)-1H-indazol-4-yl)-7-fluorobenzo[b]thiophene-3-carbonitrile and (R)-2-amino-4-(5-chloro-1-((5R,8S)-4-((S)-1-((S)-1-methylpyrrolidin-2-yl)ethoxy)-6,7,8,9-tetrahydro-5H-5,8-epoxycyclohepta[d]pyrimidin-2-yl)-1H-indazol -4-yl)-7-fluorobenzo[b]thiophene-3-carbonitrile

The title compounds were obtained from the title compounds of step 1, following a similar procedure to that described in Example 44 and 45, step 2 and step 3. Peak 1, Example 53, (8.7 mg, 18.3%) and Peak 2, Example 54 (7.0 mg, 14.7%) as an off-white solid.

Example 53: LCMS: m/z=630 [M+H]⁺, 1H NMR (400 MHz, DMSO-d₆) δ 8.67 (d, 1H), 8.04 (s, 2H), 7.91 (s, 1H), 7.72 (d, 1H), 7.29 (dd, 1H), 7.17 (t, 1H), 5.41 (p, 1H), 5.18 (d, 1H), 4.80 (t, 1H), 3.27 (dd, 1H), 3.08-2.97 (m, 1H), 2.80-2.63 (m, 2H), 2.42 (s, 3H), 2.28 (q, 1H), 2.20 (q, 1H), 2.08 (tt, 1H), 2.02-1.85 (m, 2H), 1.73 (ddd, 4H), 1.37 (d, 3H).

Example 54, LCMS: m/z=630 [M+H]⁺, 1H NMR (400 MHz, DMSO-d₆) δ 8.68 (d, 1H), 8.04 (s, 2H), 7.91 (s, 1H), 7.72 (d, 1H), 7.28 (dd, 1H), 7.17 (t, 1H), 5.43 (p, 1H), 5.18 (d, 1H), 4.80 (t, 1H), 3.27 (dd, 1H), 2.99 (d, 1H), 2.69 (t, 2H), 2.41 (s, 3H), 2.23 (dq, 2H), 2.08 (dp, 1H), 2.01-1.85 (m, 1H), 1.79-1.68 (m, 4H), 1.37 (d, 3H).

### Example 55

### [(S)-2-amino-4-(5-chloro-1-((5R,8S)-4-((S)-1-((S)-1-methylpyrrolidin-2-yl)ethoxy)-6,7,8,9-tetrahydro-5H-5,8-epoxycyclohepta[d]pyrimidin-2-yl)-1H-indazol-4-yl)-7-fluorobenzo[b]thiophene-3-carbonitrile or (R)-2-amino-4-(5-chloro-1-((5R,8S)-4-((S)-1-((S)-1-methylpyrrolidin-2-yl)ethoxy)-6,7,8,9-tetrahydro-5H-5,8-epoxycyclohepta[d]pyrimidin-2-yl)-1H-indazol-4-yl)-7-fluorobenzo[b]thiophene-3-carbonitrile] or [(S)-2-amino-4-(5-chloro-1-((5S,8R)-4-((S)-1-((S)-1-methylpyrrolidin-2-yl)ethoxy)-6,7,8,9-tetrahydro-5H-5,8-epoxycyclohepta[d]pyrimidin-2-yl)-1H-indazol-4-yl)-7-fluorobenzo[b]thiophene-3-carbonitrile or (R)-2-amino-4-(5-chloro-1-((5S,8R)-4-((S)-1-((S)-1-methylpyrrolidin-2-yl)ethoxy)-6,7,8,9-tetrahydro-5H-5,8-epoxycyclohepta[d]pyrimidin-2-yl)-1H-indazol-4-yl)-7-fluorobenzo[b]thiophene-3-carbonitrile]

### And Example 56

### [(R)-2-amino-4-(5-chloro-1-((5R,8S)-4-((S)-1-((S)-1-methylpyrrolidin-2-yl)ethoxy)-6,7,8,9-tetrahydro-5H-5,8-epoxycyclohepta[d]pyrimidin-2-yl)-1H-indazol-4-yl)-7-fluorobenzo[b]thiophene-3-carbonitrile or (S)-2-amino-4-(5-chloro-1-((5R,8S)-4-((S)-1-((S)-1-methylpyrrolidin-2-yl)ethoxy)-6,7,8,9-tetrahydro-5H-5,8-epoxycyclohepta[d]pyrimidin-2-yl)-1H-indazol-4-yl)-7-fluorobenzo[b]thiophene-3-carbonitrile] or [(R)-2-amino-4-(5-chloro-1-((5S,8R)-4-((S)-1-((S)-1-methylpyrrolidin-2-yl)ethoxy)-6,7,8,9-tetrahydro-5H-5,8-epoxycyclohepta[d]pyrimidin-2-yl)-1H-indazol-4-yl)-7-fluorobenzo[b]thiophene-3-carbonitrile or (S)-2-amino-4-(5-chloro-1-((5S,8R)-4-((S)-1-epoxycyclohepta[d]pyrimidin-2-yl)-1H-indazol-4-yl)-7-fluorobenzo[b]thiophene-3-carbonitrile]

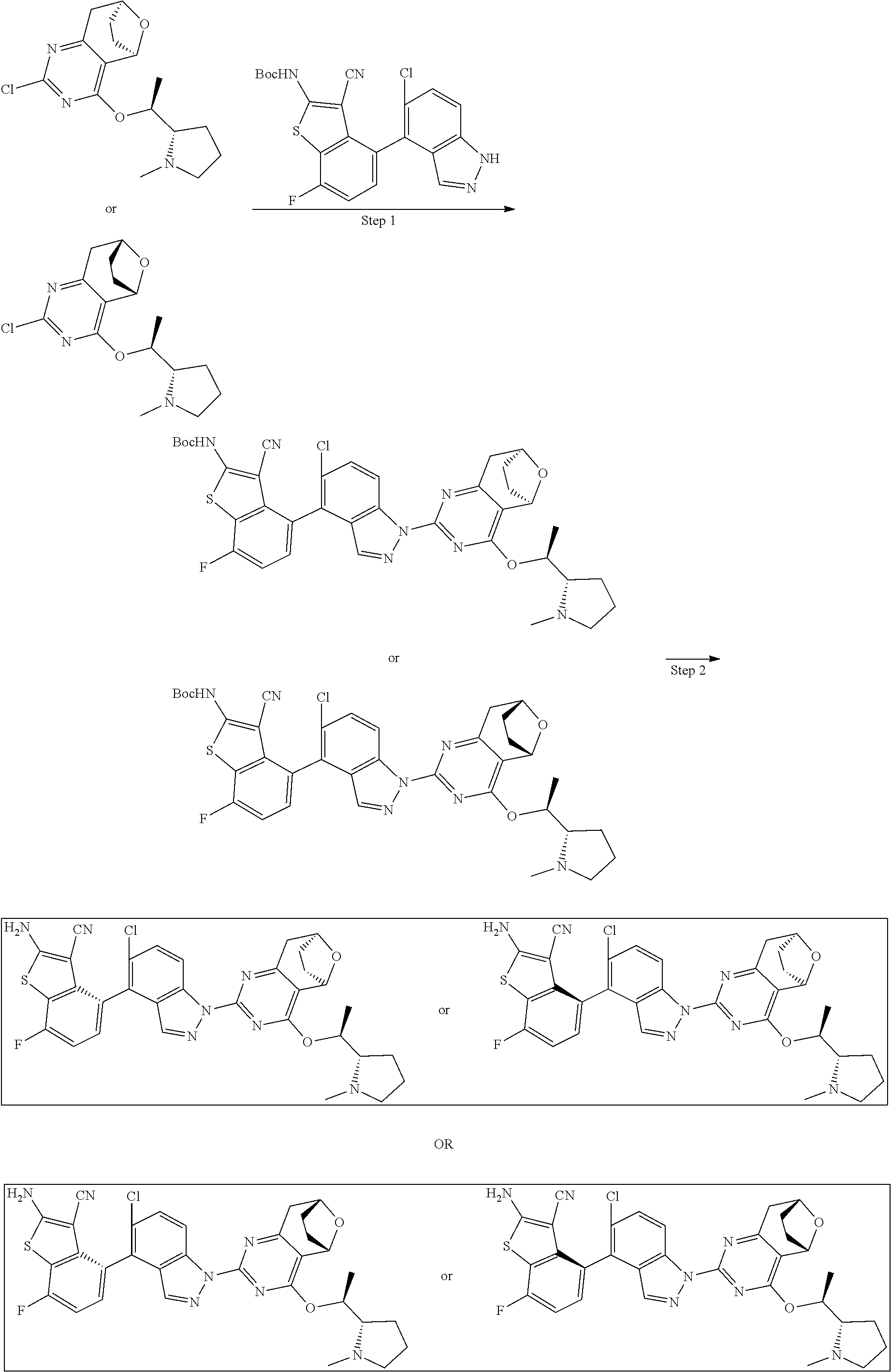

Step 1: Synthesis of tert-butyl (4-(5-chloro-1-((5R,8S)-4-((S)-1-((S)-1-methylpyrrolidin-2-yl)ethoxy)-6,7,8,9-tetrahydro-5H-5,8-epoxycyclohepta[d]pyrimidin-2-yl)-1H-indazol-4-yl)-3-cyano-7-fluorobenzo[b]thiophen-2-yl)carbamate or tert-butyl (4-(5-chloro-1-((5S,8R)-4-((S)-1-((S)-1-methylpyrrolidin-2-yl)ethoxy)-6,7,8,9-tetrahydro-5H-5,8-epoxycyclohepta[d]pyrimidin-2-yl)-1H-indazol-4-yl)-3-cyano-7-fluorobenzo[b]thiophen-2-yl)carbamate

The title compound was obtained as a yellow solid, 60 mg, 60%, from Intermediate 3B and Intermediate 16 following a similar procedure to that described in Example 29 and 30, step2.

Step 2: Synthesis of [(S)-2-amino-4-(5-chloro-1-((5R,8S)-4-((S)-1-((S)-1-methylpyrrolidin-2-yl)ethoxy)-6,7,8,9-tetrahydro-5H-5,8-epoxycyclohepta[d]pyrimidin-2-yl)-1H-indazol-4-yl)-7-fluorobenzo[b]thiophene-3-carbonitrile or (R)-2-amino-4-(5-chloro-1-((5R,8S)-4-((S)-1-((S)-1-methylpyrrolidin-2-yl)ethoxy)-6,7,8,9-tetrahydro-5H-5,8-epoxycyclohepta[d]pyrimidin-2-yl)-1H-indazol-4-yl)-7-fluorobenzo[b]thiophene-3-carbonitrile] and [(S)-2-amino-4-(5-chloro-1-((5S,8R)-4-((S)-1-((S)-1-methylpyrrolidin-2-yl)ethoxy)-6,7,8,9-tetrahydro-5H-5,8-epoxycyclohepta[d]pyrimidin-2-yl)-1H-indazol-4-yl)-7-fluorobenzo[b]thiophene-3-carbonitrile or (R)-2-amino-4-(5-chloro-1-((5S,8R)-4-((S)-1-((S)-1-methylpyrrolidin-2-yl)ethoxy)-6,7,8,9-tetrahydro-5H-5,8-epoxycyclohepta[d]pyrimidin-2-yl)-1H-indazol-4-yl)-7-fluorobenzo[b]thiophene-3-carbonitrile]

The title compounds were obtained from the title compound of step 1, following a similar procedure to that described in Example 44 and 45, step 2 and step 3.

Peak 1, Example 55, (6.8 mg, 14.3%) as an off-white solid. LCMS: m/z=630 [M+H]⁺, 1H NMR (400 MHz, DMSO) δ 8.67 (d, 1H), 8.04 (s, 2H), 7.91 (s, 1H), 7.72 (d, 1H), 7.29 (dd, 1H), 7.17 (dd, 1H), 5.46 (p, 1H), 5.15 (d, 1H), 4.87-4.74 (m, 1H), 3.28 (dd, 1H), 3.00 (ddd, 1H), 2.75-2.62 (m, 2H), 2.41 (s, 3H), 2.31-2.16 (m, 2H), 2.09 (tt, 1H), 2.01-1.84 (m, 2H), 1.83-1.65 (m, 4H), 1.33 (d, 3H).

and Peak 2, Example 56 (4 mg, 8.43%) as an off-white solid. LCMS: m/z=630 [M+H]⁺, 1H NMR (400 MHz, DMSO) δ 8.67 (d, 1H), 8.04 (s, 2H), 7.90 (s, 1H), 7.72 (d, 1H), 7.30 (dd, 1H), 7.17 (t, 1H), 5.49 (p, 1H), 5.15 (d, 1H), 4.80 (t, 1H), 3.28 (dd, 1H), 2.99 (dq, 1H), 2.78-2.63 (m, 2H), 2.41 (s, 3H), 2.31-2.15 (m, 2H), 2.10 (dq, 1H), 2.04-1.82 (m, 2H), 1.84-1.63 (m, 4H), 1.34 (d, 3H).

### Example 57

### (S)-2-amino-4-(5-chloro-3-methyl-1-(8-methyl-4-((S)-1-((S)-1-methylpyrrolidin-2-yl)ethoxy)-7,8-dihydro-6H-pyrimido[5,4-b][1,4]oxazin-2-yl)-1H-indazol-4-yl)-7-fluorobenzo[b]thiophene-3-carbonitrile or (R)-2-amino-4-(5-chloro-3-methyl-1-(8-methyl-4-((S)-1-((S)-1-methylpyrrolidin-2-yl)ethoxy)-7,8-dihydro-6H-pyrimido[5,4-b][1,4]oxazin-2-yl)-1H-indazol-4-yl)-7-fluorobenzo[b]thiophene-3-carbonitrile

### And Example 58

### (R)-2-amino-4-(5-chloro-3-methyl-1-(8-methyl-4-((S)-1-((S)-1-methylpyrrolidin-2-yl)ethoxy)-7,8-dihydro-6H-pyrimido[5,4-b][1,4]oxazin-2-yl)-1H-indazol-4-yl)-7-fluorobenzo[b]thiophene-3-carbonitrile or (S)-2-amino-4-(5-chloro-3-methyl-1-(8-methyl-4-((S)-1-((S)-1-methylpyrrolidin-2-yl)ethoxy)-7,8-dihydro-6H-pyrimido[5,4-b][1,4]oxazin-2-yl)-1H-indazol-4-yl)-7-fluorobenzo[b]thiophene-3-carbonitrile

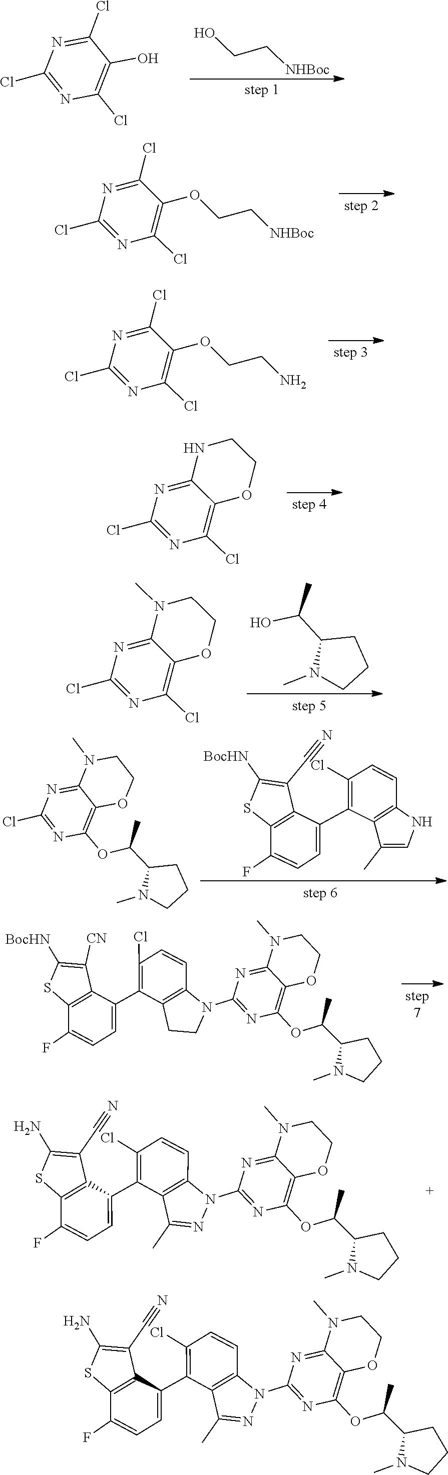

Step 1: Synthesis of tert-butyl(2-((2,4,6-trichloropyrimidin-5-yl)oxy)ethyl)carbamate

To a stirred solution of trichloropyrimidin-5-ol (2.0 g, 10.0 mmol) and tert-butyl N-(2-hydroxyethyl)carbamate (3.22 g, 20.0 mmol) in THF (30 mL) were added PPh₃ (3.93 g, 15.0 mmol) and DIAD (3.03 g, 15.0 mmol) at 0° C. and the reaction mixture was stirred overnight at rt. Water (100 mL) was added and the resulting solution was extracted with DCM (3×100 mL). The combined organic layers were dried over Na₂SO₄ and concentrated. The crude product was purified by silica gel column with PE:EtOAc=8:1 to give the title compound (2.87 g, 84%) as a white solid.

Step 2: Synthesis of 2-((2,4,6-trichloropyrimidin-5-yl)oxy)ethan-1-amine trifluoroacetate

To a stirred solution of the title compound of step 1 (1.6 g, 4.67 mmol) in DCM (15 mL) was added TFA (3 mL) and the reaction mixture was stirred overnight at rt. The mixture was concentrated in vacuo and the crude product was purified by silica gel column chromatography with DCM:MeOH=8:1 to give the title compound (1.0 g, 88%) as a colorless oil. LCMS m/z=242 [M+H]⁺

Step 3: Synthesis of 2,4-dichloro-7,8-dihydro-6H-pyrimido[5,4-b][1,4]oxazine

To a stirred solution of the title compound of step 2 (1.0 g, 4.12 mmol) in dioxane (10 mL) was added DIEA (1.58 g, 12.3 mmol) and the reaction was stirred for 18 h at 80° C. The mixture was concentrated in vacuo and the crude product was purified by silica gel column with PE: EtOAc=2:1 to give the title compound (740 mg, 87%) as a white solid. LCMS: m/z=206 [M+H]⁺

Step 4: Synthesis of 2,4-dichloro-8-methyl-7,8-dihydro-6H-pyrimido[5,4-b][1,4]oxazine.

To a stirred solution of the title compound from step 3 (300 mg, 1.45 mmol) in THF (8 mL) were added MeI (245 mg, 1.73 mmol) and KOtBu (325 mg, 2.90 mmol) at 0° C. The reaction mixture was stirred for 2 h at rt. Water (15 mL) was added and the resulting solution was extracted with DCM (3×15 mL). The combined organic layers were dried over Na₂SO₄ and concentrated. The crude product was purified by Prep-TLC with PE:EtOAc=2:1 to give the title compound (300 mg, 94%) as a white solid. LCMS m/z=220 [M+H]+

Step 5: Synthesis of 2-chloro-8-methyl-4-((S)-1-((S)-1-methylpyrrolidin-2-yl)ethoxy)-7,8-dihydro-6H-pyrimido[5,4-b][1,4]oxazine

The title compound was obtained as a yellow oil, 150 mg, 35%, from the title compound from step 4 and (S)-1-((S)-1-methylpyrrolidin-2-yl)ethan-1-ol, following a similar procedure to that described in Example 47 and 48, step 3. LCMS m/z=313 [M+H]+

Step 6: Synthesis of tert-butyl (4-(5-chloro-3-methyl-1-(8-methyl-4-((S)-1-((S)-1-methylpyrrolidin-2-yl)ethoxy)-7,8-dihydro-6H-pyrimido[5,4-b][1,4]oxazin-2-yl)-1H-indazol-4-yl)-3-cyano-7-fluorobenzo[b]thiophen-2-yl)carbamate

The title compound was obtained as a yellow solid, 200 mg, 71%, from the title compound from step 5 and Intermediate 15, following a similar procedure to that described in Example 29 and 30, step 2. LCMS m/z=733 [M+H]⁺

Step 7: Synthesis of (S)-2-amino-4-(5-chloro-3-methyl-1-(8-methyl-4-((S)-1-((S)-1-methylpyrrolidin-2-yl)ethoxy)-7,8-dihydro-6H-pyrimido[5,4-b][1,4]oxazin-2-yl)-1H-indazol-4-yl)-7-fluorobenzo[b]thiophene-3-carbonitrile and (R)-2-amino-4-(5-chloro-3-methyl-1-(8-methyl-4-((S)-1-((S)-1-methylpyrrolidin-2-yl)ethoxy)-7,8-dihydro-6H-pyrimido[5,4-b][1,4]oxazin-2-yl)-1H-indazol-4-yl)-7-fluorobenzo[b]thiophene-3-carbonitrile

The title compounds were obtained as Peak 1, Example 57 (66.6 mg, 39%) as an off-white solid and Peak 2, Example 58 (58 mg, 34%) as an off-white solid, from the title compound of step 6, following a similar procedure to that described in Example 29 and 30, step 3.

Example 57: LCMS: m/z=633 [M+H]⁺; 1H NMR (400 MHz, DMSO-d₆) δ 8.57 (d, 1H), 8.03 (s, 2H), 7.64 (d, 1H), 7.23 (dd, 1H), 7.15 (dd, 1H), 5.29 (p, 1H), 4.20 (d, 2H), 3.56 (dd, 2H), 3.21 (s, 3H), 3.06-2.95 (m, 1H), 2.72-2.63 (m, 1H), 2.43 (s, 3H), 2.29-2.19 (m, 1H), 1.88-1.74 (m, 1H), 1.72 (s, 3H), 1.70-1.65 (m, 3H), 1.28 (d, 3H).

Example 58: LCMS: m/z=633 [M+H]⁺; 1H NMR (400 MHz, DMSO-d₆) δ 8.58 (d, 1H), 8.04 (s, 2H), 7.65 (d, 1H), 7.23 (dd, 1H), 7.15 (dd, 1H), 5.30 (p, 1H), 4.20 (t, 2H), 3.56 (t, 2H), 3.21 (s, 3H), 3.06-2.97 (m, 1H), 2.73 (dt, 1H), 2.46 (s, 3H), 2.28 (td, 1H), 1.89-1.73 (m, 1H), 1.73 (s, 3H), 1.76-1.63 (m, 3H), 1.30 (d, 3H).

### Example 59

### [(S)-2-amino-4-(5-chloro-1-((R)-8-methyl-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (R)-2-amino-4-(5-chloro-1-((R)-8-methyl-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile] or [(S)-2-amino-4-(5-chloro-1-((S)-8-methyl-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (R)-2-amino-4-(5-chloro-1-((S)-8-methyl-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile]

### And Example 60

### [(R)-2-amino-4-(5-chloro-1-((R)-8-methyl-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (S)-2-amino-4-(5-chloro-1-((R)-8-methyl-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile] or [(R)-2-amino-4-(5-chloro-1-((S)-8-methyl-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (S)-2-amino-4-(5-chloro-1-((S)-8-methyl-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile]

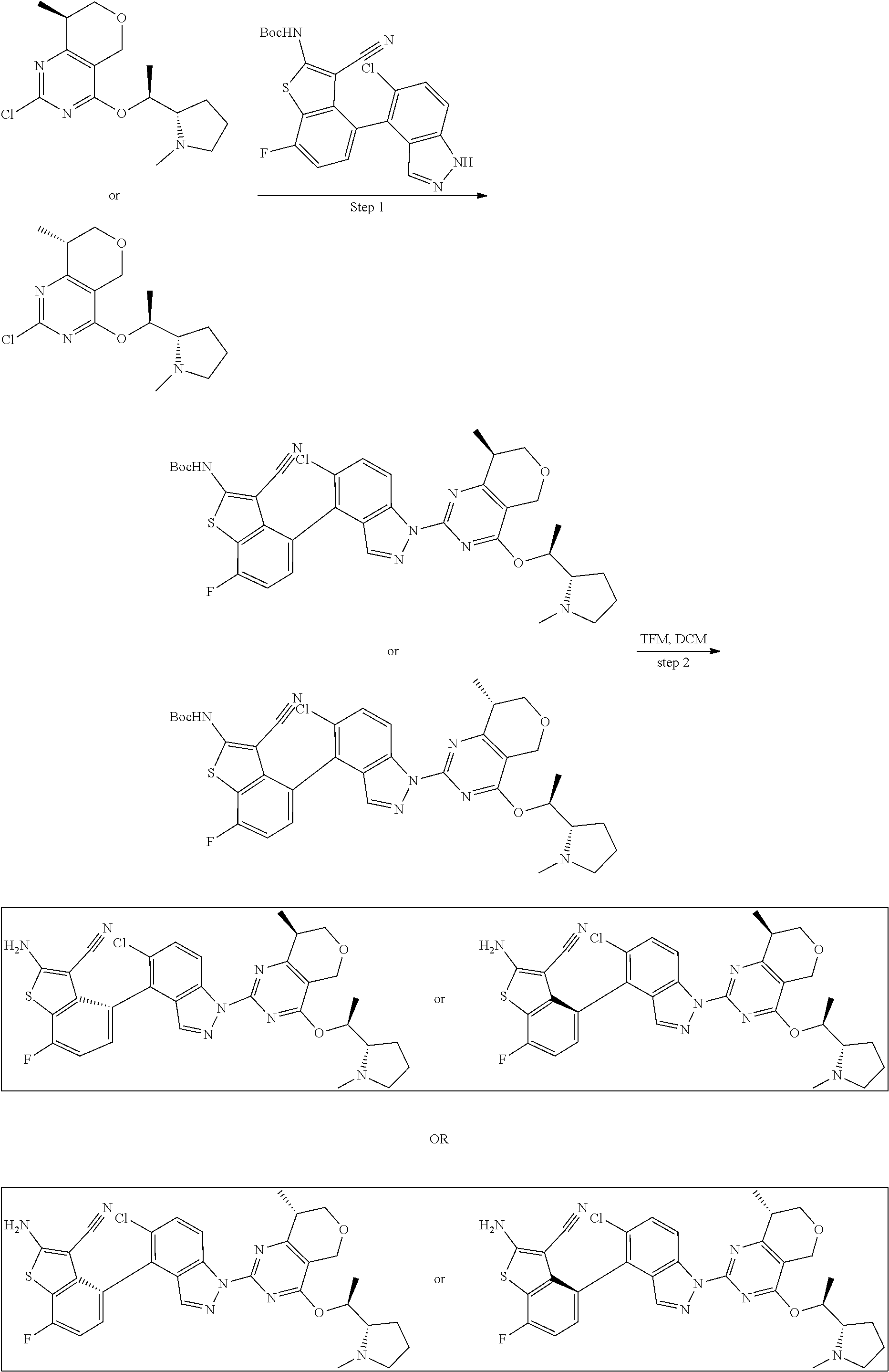

Step 1: Synthesis of tert-butyl (4-(5-chloro-1-((R)-8-methyl-4-((S)-1-((S)-1-methylpyrrolidin-2-yl)ethoxy)-7,8-dihydro-5H-pyrano[4,3-d]pyrimidin-2-yl)-1H-indazol-4-yl)-3-cyano-7-fluorobenzo[b]thiophen-2-yl)carbamate or tert-butyl (4-(5-chloro-1-((S)-8-methyl-4-((S)-1-((S)-1-methylpyrrolidin-2-yl)ethoxy)-7,8-dihydro-5H-pyrano[4,3-d]pyrimidin-2-yl)-1H-indazol-4-yl)-3-cyano-7-fluorobenzo[b]thiophen-2-yl)carbamate

The title compound was obtained as a yellow solid, 100 mg, 44% from Intermediate 5A and Intermediate 16, following a similar procedure to that described in Example 47 and 48, step 5. LCMS: m/z=718 [M+H]⁺.

Step 2: Synthesis of 2-amino-4-((S)-5-chloro-1-((R)-8-methyl-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or 2-amino-4-((S)-5-chloro-1-((S)-8-methyl-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile and 2-amino-4-((R)-5-chloro-1-((R)-8-methyl-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or 2-amino-4-((R)-5-chloro-1-((S)-8-methyl-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

The title compounds were obtained from the title compound from step 1, following a similar procedure to that described in Example 29 and 30 step 3.

Peak 1, Example 59, 19.3 mg, 39% as a white solid. LCMS: m/z=618 [M+H]⁺, 1H NMR (400 MHz, DMSO-d₆) δ 8.70 (d, 1H), 8.04 (s, 2H), 7.94 (s, 1H), 7.76 (d, 1H), 7.28 (dd, 1H), 7.21-7.14 (m, 1H), 5.46-5.39 (m, 1H), 4.69-4.56 (m, 2H), 4.06 (dd, 1H), 3.69 (dd, 1H), 3.01 (q, 2H), 2.71-2.67 (m, 1H), 2.41 (s, 3H), 2.31-2.26 (m, 1H), 1.91-1.84 (m, 1H), 1.81-1.64 (m, 3H), 1.37 (d, 3H), 1.33 (d, 3H).

Peak 2, Example 60, 18.7 mg, 37% as a white solid. LCMS: m/z=618 [M+H]⁺, 1H NMR (400 MHz, DMSO-d₆) δ 8.69 (d, 1H), 8.03 (s, 2H), 7.93 (s, 1H), 7.75 (d, 1H), 7.28 (dd, 1H), 7.21-7.14 (m, 1H), 5.46-5.39 (m, 1H), 4.68-4.56 (m, 2H), 4.06 (dd, 1H), 3.68 (dd, 1H), 3.07-2.96 (m, 2H), 2.74-2.66 (m, 1H), 2.40 (s, 3H), 2.27 (q, 1H), 1.94-1.80 (m, 1H), 1.78-1.63 (m, 3H), 1.35 (d, 3H), 1.33 (d, 3H).

### Example 61

### 2-amino-4-(5-chloro-1-((S)-6-hydroxy-6-methyl-4-((S)-1-((S)-1-methylpyrrolidin-2-yl)ethoxy)-5,6,7,8-tetrahydroquinazolin-2-yl)-3-methyl-1H-indazol-4-yl)-7-fluorobenzo[b]thiophene-3-carbonitrile or 2-amino-4-(5-chloro-1-((R)-6-hydroxy-6-methyl-4-((S)-1-((S)-1-methylpyrrolidin-2-yl)ethoxy)-5,6,7,8-tetrahydroquinazolin-2-yl)-3-methyl-1H-indazol-4-yl)-7-fluorobenzo[b]thiophene-3-carbonitrile

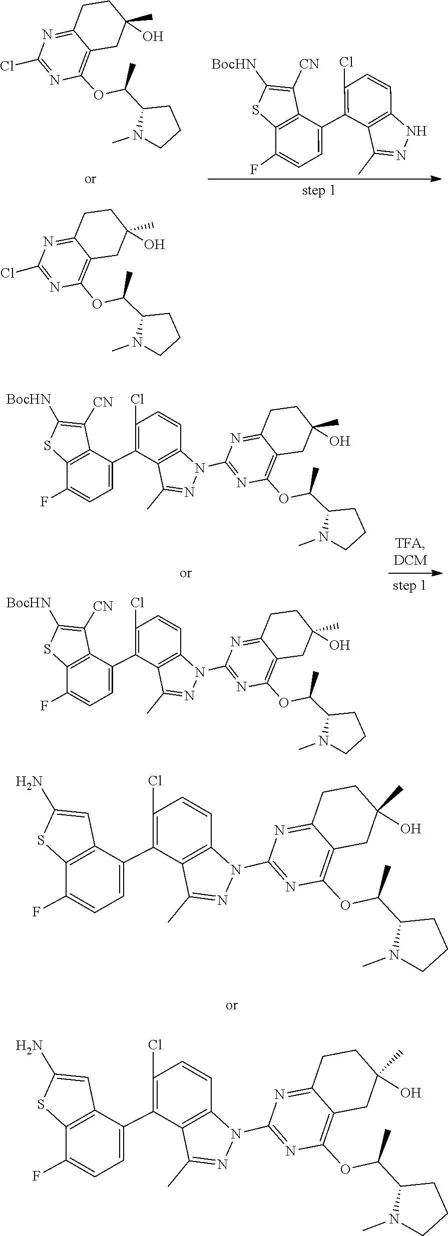

Step 1: Synthesis of tert-butyl (4-(5-chloro-1-((S)-6-hydroxy-6-methyl-4-((S)-1-((S)-1-methylpyrrolidin-2-yl)ethoxy)-5,6,7,8-tetrahydroquinazolin-2-yl)-3-methyl-1H-indazol-4-yl)-3-cyano-7-fluorobenzo[b]thiophen-2-yl)carbamate or tert-butyl (4-(5-chloro-1-((R)-6-hydroxy-6-methyl-4-((S)-1-((S)-1-methylpyrrolidin-2-yl)ethoxy)-5,6,7,8-tetrahydroquinazolin-2-yl)-3-methyl-1H-indazol-4-yl)-3-cyano-7-fluorobenzo[b]thiophen-2-yl)carbamate

The title compound was obtained as a yellow solid, 20 mg, 29%, from Intermediate 4A and Intermediate 15, following a similar procedure to that described in Example 38 and 39, step 1. LCMS m/z=746 [M+H]+

Step 2: Synthesis of 2-amino-4-(5-chloro-1-((S)-6-hydroxy-6-methyl-4-((S)-1-((S)-1-methylpyrrolidin-2-yl)ethoxy)-5,6,7,8-tetrahydroquinazolin-2-yl)-3-methyl-1H-indazol-4-yl)-7-fluorobenzo[b]thiophene-3-carbonitrile or 2-amino-4-(5-chloro-1-((R)-6-hydroxy-6-methyl-4-((S)-1-((S)-1-methylpyrrolidin-2-yl)ethoxy)-5,6,7,8-tetrahydroquinazolin-2-yl)-3-methyl-1H-indazol-4-yl)-7-fluorobenzo[b]thiophene-3-carbonitrile

The title compound was obtained as a white solid, 7.3 mg, 42%, from the title compound from step 1, following a similar procedure to that described in Example 38 and 39, step 2 and step 3. LCMS: m/z=646 [M+H]⁺, 1H NMR (400 MHz, DMSO-d₆) δ 8.68 (t, 1H), 8.04 (s, 2H), 7.68 (d, 1H), 7.25 (dd, 1H), 7.16 (dd, 1H), 5.42 (p, 1H), 4.59 (s, 1H), 3.01-2.94 (m, 2H), 2.83-2.76 (m, 1H), 2.76-2.61 (m, 2H), 2.63-2.59 (m, 1H), 2.40 (d, 3H), 2.27-2.19 (m, 1H), 1.88-1.79 (m, 2H), 1.75-1.65 (m, 7H), 1.34 (dd, 3H), 1.29 (s, 3H).

### Example 62

### [(S)-2-amino-4-(5-chloro-1-((R)-6-hydroxy-6-methyl-4-((S)-1-((S)-1-methylpyrrolidin-2-yl)ethoxy)-5,6,7,8-tetrahydroquinazolin-2-yl)-3-methyl-1H-indazol-4-yl)-7-fluorobenzo[b]thiophene-3-carbonitrile or (R)-2-amino-4-(5-chloro-1-((R)-6-hydroxy-6-methyl-4-((S)-1-((S)-1-methylpyrrolidin-2-yl)ethoxy)-5,6,7,8-tetrahydroquinazolin-2-yl)-3-methyl-1H-indazol-4-yl)-7-fluorobenzo[b]thiophene-3-carbonitrile] or [(S)-2-amino-4-(5-chloro-1-((S)-6-hydroxy-6-methyl-4-((S)-1-((S)-1-methylpyrrolidin-2-yl)ethoxy)-5,6,7,8-tetrahydroquinazolin-2-yl)-3-methyl-1H-indazol-4-yl)-7-fluorobenzo[b]thiophene-3-carbonitrile or (R)-2-amino-4-(5-chloro-1-((S)-6-hydroxy-6-methyl-4-((S)-1-((S)-1-methylpyrrolidin-2-yl)ethoxy)-5,6,7,8-tetrahydroquinazolin-2-yl)-3-methyl-1H-indazol-4-yl)-7-fluorobenzo[b]thiophene-3-carbonitrile]

### And Example 63

### [(R)-2-amino-4-(5-chloro-1-((R)-6-hydroxy-6-methyl-4-((S)-1-((S)-1-methylpyrrolidin-2-yl)ethoxy)-5,6,7,8-tetrahydroquinazolin-2-yl)-3-methyl-1H-indazol-4-yl)-7-fluorobenzo[b]thiophene-3-carbonitrile or (S)-2-amino-4-(5-chloro-1-((R)-6-hydroxy-6-methyl-4-((S)-1-((S)-1-methylpyrrolidin-2-yl)ethoxy)-5,6,7,8-tetrahydroquinazolin-2-yl)-3-methyl-1H-indazol-4-yl)-7-fluorobenzo[b]thiophene-3-carbonitrile] or [(R)-2-amino-4-(5-chloro-1-((S)-6-hydroxy-6-methyl-4-((S)-1-((S)-1-methylpyrrolidin-2-yl)ethoxy)-5,6,7,8-tetrahydroquinazolin-2-yl)-3-methyl-1H-indazol-4-yl)-7-fluorobenzo[b]thiophene-3-carbonitrile or (S)-2-amino-4-(5-chloro-1-((S)-6-hydroxy-6-methyl-4-((S)-1-((S)-1-methylpyrrolidin-2-yl)ethoxy)-5,6,7,8-tetrahydroquinazolin-2-yl)-3-methyl-1H-indazol-4-yl)-7-fluorobenzo[b]thiophene-3-carbonitrile]

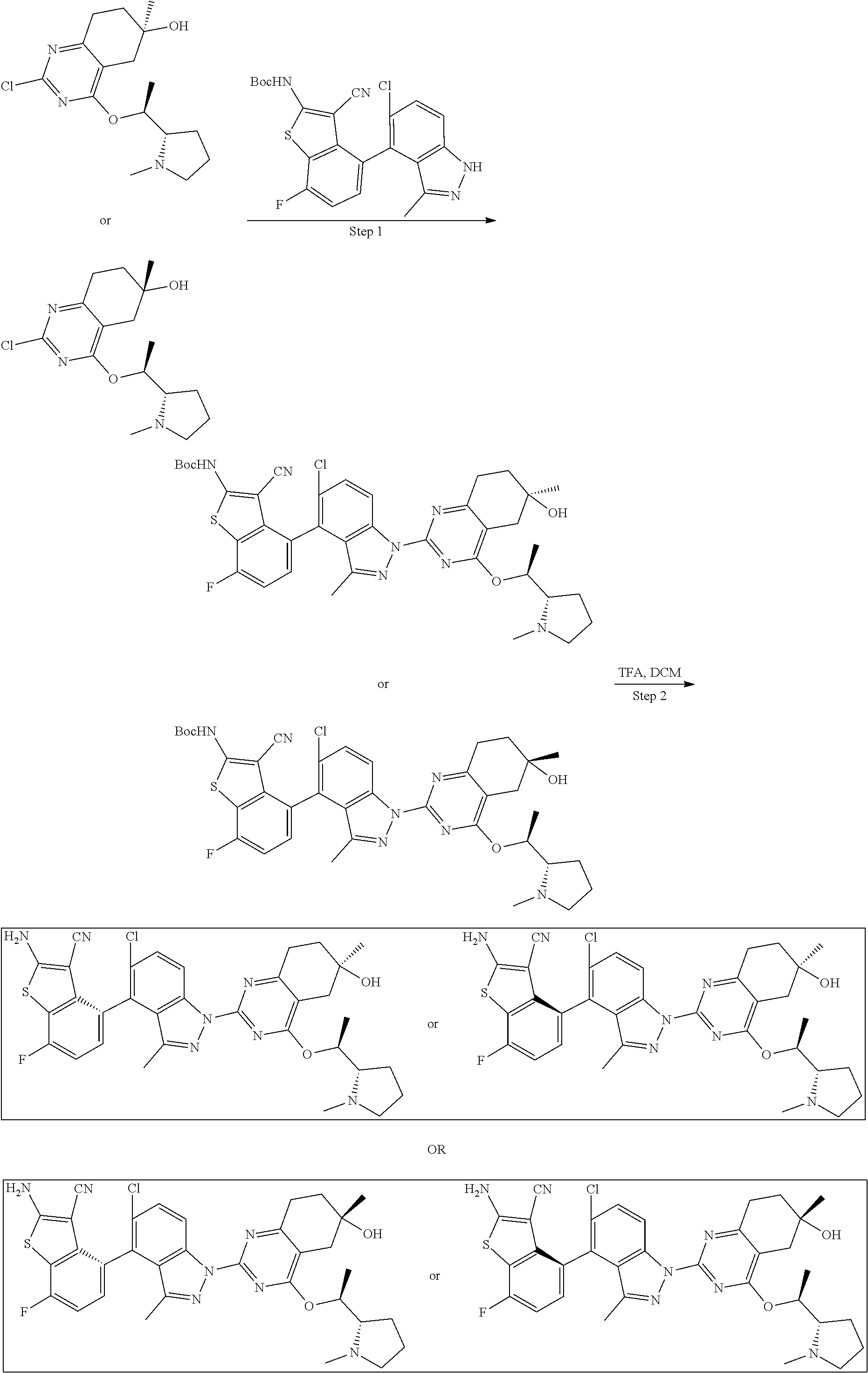

Step 1: Synthesis of tert-butyl (4-(5-chloro-1-((R)-6-hydroxy-6-methyl-4-((S)-1-((S)-1-methylpyrrolidin-2-yl)ethoxy)-5,6,7,8-tetrahydroquinazolin-2-yl)-3-methyl-1H-indazol-4-yl)-3-cyano-7-fluorobenzo[b]thiophen-2-yl)carbamate or tert-butyl (4-(5-chloro-1-((S)-6-hydroxy-6-methyl-4-((S)-1-((S)-1-methylpyrrolidin-2-yl)ethoxy)-5,6,7,8-tetrahydroquinazolin-2-yl)-3-methyl-1H-indazol-4-yl)-3-cyano-7-fluorobenzo[b]thiophen-2-yl)carbamate

The title compound was obtained as a yellow solid, 30 mg, 44%, from Intermediate 4B and Intermediate 16, following a similar procedure to that described in Example 29 and 30, step 2. LCMS m/z=746 [M+H]+

Step 2: Synthesis of (S)-2-amino-4-(5-chloro-1-((R)-6-hydroxy-6-methyl-4-((S)-1-((S)-1-methylpyrrolidin-2-yl)ethoxy)-5,6,7,8-tetrahydroquinazolin-2-yl)-3-methyl-1H-indazol-4-yl)-7-fluorobenzo[b]thiophene-3-carbonitrile or (R)-2-amino-4-(5-chloro-1-((R)-6-hydroxy-6-methyl-4-((S)-1-((S)-1-methylpyrrolidin-2-yl)ethoxy)-5,6,7,8-tetrahydroquinazolin-2-yl)-3-methyl-1H-indazol-4-yl)-7-fluorobenzo[b]thiophene-3-carbonitrile

The title compounds were obtained from the title compound from step 1, following a similar procedure to that described in Example 29 and 30, step 3.

Peak 1, Example 62, 8.0 mg, 46% as a white solid. LCMS: m/z=646 [M+H]⁺, 1H NMR (400 MHz, DMSO-d₆) δ 8.68 (d, 1H), 8.04 (s, 2H), 7.68 (d, 1H), 7.26 (dd, 1H), 7.16 (t, 1H), 5.44 (p, 1H), 4.61 (s, 1H), 2.98 (dt, 2H), 2.79-2.75 (m, 2H), 2.65 (dt, 2H), 2.57 (d, 1H), 2.40 (s, 3H), 2.21 (q, 1H), 1.84 (td, 3H), 1.80-1.66 (m, 6H), 1.39 1 1.26 (m, 6H).

And Peak 2, Example 63. (8.1 mg, 47%) as a white solid. LCMS: m/z=646 [M+H]⁺, 1H NMR (400 MHz, DMSO-d₆) δ 8.68 (d, 1H), 8.04 (s, 2H), 7.68 (d, 1H), 7.25 (dd, 1H), 7.16 (dd, 1H), 5.46 (p, 1H), 4.59 (s, 1H), 3.06-2.95 (m, 2H), 2.77-2.74 (m, 1H), 2.66 (dt, 1H), 2.55-2.50 (m, 1H), 2.41 (s, 3H), 2.21 (q, 1H), 1.89-1.77 (m, 3H), 1.77-1.65 (m, 6H), 1.33 (d, 3H), 1.29 (s, 3H).

### Example 64

### (S)-2-amino-4-(5-chloro-3-fluoro-1-(4-((S)-1-((2S,4R)-4-fluoro-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (R)-2-amino-4-(5-chloro-3-fluoro-1-(4-((S)-1-((2S,4R)-4-fluoro-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

### And Example 65

### (R)-2-amino-4-(5-chloro-3-fluoro-1-(4-((S)-1-((2S,4R)-4-fluoro-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (S)-2-amino-4-(5-chloro-3-fluoro-1-(4-((S)-1-((2S,4R)-4-fluoro-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

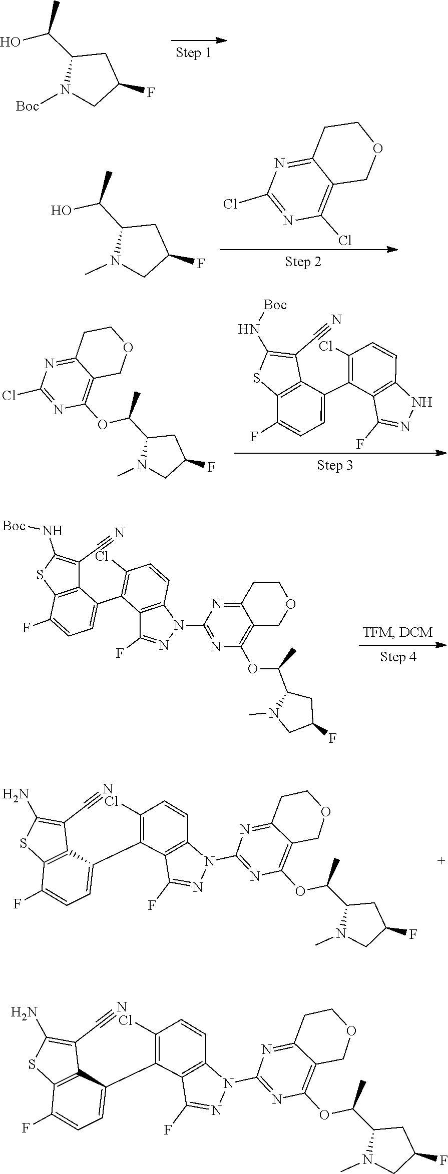

Step 1: Synthesis of (S)-1-((2S,4R)-4-fluoro-1-methylpyrrolidin-2-yl)ethan-1-ol

A solution of tert-butyl(2S,4R)-4-fluoro-2-((S)-1-hydroxyethyl)pyrrolidine-1-carboxylate (500 mg, 2.14 mmol) in THF (10 mL) was pumped by Pump 1 (3.2 mL/min) to flow reactor 1 (PFA, Coils reactor, 3.175 mm (⅛″), 64 mL, 60° C.). A solution of LiAlH₄ (2.5 M, 2.57 mL) filled to 10 mL with THE was pumped by Pump 2 (3.2 mL/min) to flow reactor 1. The residence time of flow reactor 1 was 10 min. The resulting mixture was collected with a bottle (quenched with 2 g 10H₂O·Na₂SO₄ at 0° C.). Pump 1 and Pump 2 were started simultaneously. The reaction was quenched with water (1 mL) and diluted with THF (10 mL). The mixture was filtered, and the filtrate was concentrated in vacuo. The residue was purified by column chromatography (SiO₂, EtOAc: MeOH: NH₄OH=10:1: 0.01) to give the title compound (240 mg, 76.1%) as colorless oil. 1H NMR (400 MHz, CDCl₃) δ ppm 5.32-5.04 (m, 1H), 3.52-3.39 (m, 1H), 3.32-3.16 (m, 1H), 2.95-2.74 (m, 2H), 2.60 (s, 3H), 2.32-2.17 (m, 1H), 1.83-1.76 (m, 1H), 1.73-1.68 (m, 1H), 1.18 (d, 3H).

Step 2: Synthesis of 2-chloro-4-((S)-1-((2S,4R)-4-fluoro-1-methylpyrrolidin-2-yl)ethoxy)-7,8-dihydro-5H-pyrano[4,3-d]pyrimidine

The title compound was obtained as a solid, 160 mg, 36.9%, from the title compound of step 1 and Intermediate 1, step 1, following a similar procedure to that described in Intermediate 2. LCMS m/z=316 [M+H]+

Step 3: Synthesis of tert-butyl (4-(5-chloro-3-fluoro-1-(4-((S)-1-((2S,4R)-4-fluoro-1-methylpyrrolidin-2-yl)ethoxy)-7,8-dihydro-5H-pyrano[4,3-d]pyrimidin-2-yl)-1H-indazol-4-yl)-3-cyano-7-fluorobenzo[b]thiophen-2-yl)carbamate

The title compound was obtained as a yellow solid 240 mg, 72.4%, from the title compound from step 2 and Intermediate 14, following a similar procedure to that described in Example 3 and 4, step 1. LCMS m/z=740 [M+H]+

Step 4: Synthesis of (S)-2-amino-4-(5-chloro-3-fluoro-1-(4-((S)-1-((2S,4R)-4-fluoro-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile and (R)-2-amino-4-(5-chloro-3-fluoro-1-(4-((S)-1-((2S,4R)-4-fluoro-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

The title compounds were obtained from the title compound of step 3, following a similar procedure to that described in Example 25 and 26, step 3.

Peak 1, Example 64, (41.0 mg, 23.4%) as white solid. LCMS m/z=640 [M+H]+ 1H NMR (400 MHz, DMSO-d₆) δ ppm 10.92-10.17 (m, 1H), 8.72 (dd, 1H), 7.87 (d, 1H), 7.32 (dd, 1H), 7.16 (t, 1H), 5.48-5.41 (m, 2H), 4.64-4.62 (m, 2H), 4.32-3.56 (m, 4H), 3.10-2.80 (m, 4H), 2.72-2.52 (m, 2H), 2.44-2.38 (m, 2H), 1.49-1.35 (m, 3H).

Peak 2, Example 65, (51.0 mg, 29.2%) as white solid. LCMS m/z=640 [M+H]+ 1H NMR (400 MHz, DMSO-d₆) δ ppm 10.76-10.30 (m, 1H), 8.71 (dd, 1H), 7.86 (d, 1H), 7.32 (dd, 1H), 7.16 (t, 1H), 5.51-5.48 (m, 2H), 4.66-4.61 (m, 3H), 4.32-3.49 (m, 3H), 3.15-2.79 (m, 4H), 2.7-2.53 (m, 1H), 2.49-1.73 (m, 3H), 1.55-1.27 (m, 3H).

### Example 66

### (S)-2-amino-4-(5-chloro-1-(7,7-dimethyl-4-((S)-1-((S)-1-methylpyrrolidin-2-yl)ethoxy)-5,7-dihydrofuro[3,4-d]pyrimidin-2-yl)-3-methyl-1H-indazol-4-yl)-7-fluorobenzo[b]thiophene-3-carbonitrile or (R)-2-amino-4-(5-chloro-1-(7,7-dimethyl-4-((S)-1-((S)-1-methylpyrrolidin-2-yl)ethoxy)-5,7-dihydrofuro[3,4-d]pyrimidin-2-yl)-3-methyl-1H-indazol-4-yl)-7-fluorobenzo[b]thiophene-3-carbonitrile and

### Example 67

### (R)-2-amino-4-(5-chloro-1-(7,7-dimethyl-4-((S)-1-((S)-1-methylpyrrolidin-2-yl)ethoxy)-5,7-dihydrofuro[3,4-d]pyrimidin-2-yl)-3-methyl-1H-indazol-4-yl)-7-fluorobenzo[b]thiophene-3-carbonitrile or (S)-2-amino-4-(5-chloro-1-(7,7-dimethyl-4-((S)-1-((S)-1-methylpyrrolidin-2-yl)ethoxy)-5,7-dihydrofuro[3,4-d]pyrimidin-2-yl)-3-methyl-1H-indazol -4-yl)-7-fluorobenzo[b]thiophene-3-carbonitrile

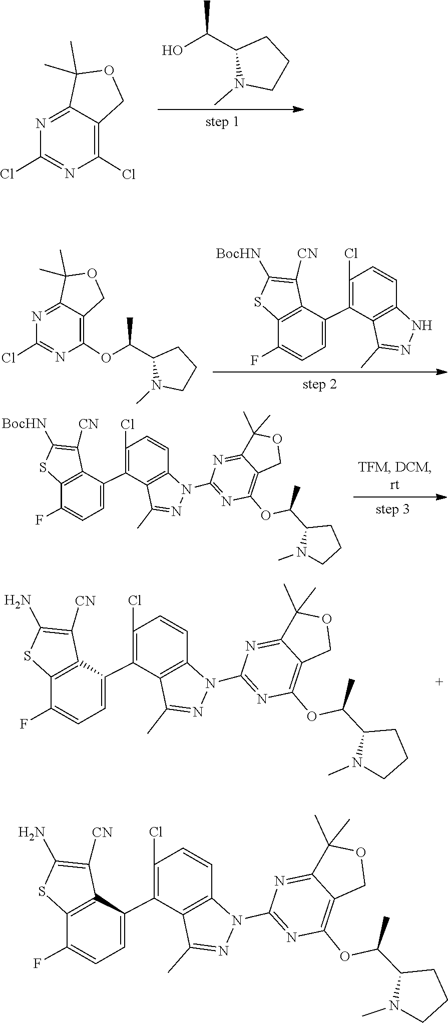

Step 1: Synthesis of 2-chloro-7,7-dimethyl-4-((S)-1-((S)-1-methylpyrrolidin-2-yl)ethoxy)-5,7-dihydrofuro[3,4-d]pyrimidine

The title compound was obtained as a yellow solid, 180 mg, 63%, from(S)-1-((S)-1-methylpyrrolidin-2-yl)ethan-1-ol and 2,4-dichloro-7,7-dimethyl-5H, 7H-furo[3,4-d]pyrimidine, following a similar procedure to that described in Intermediate 4A and 4B, step 2. LCMS m/z=312 [M+H]+

Step 2: Synthesis of tert-butyl (4-(5-chloro-1-(7,7-dimethyl-4-((S)-1-((S)-1-methylpyrrolidin-2-yl)ethoxy)-5,7-dihydrofuro[3,4-d]pyrimidin-2-yl)-3-methyl-1H-indazol-4-yl)-3-cyano-7-fluorobenzo[b]thiophen-2-yl)carbamate

The title compound was obtained as a yellow solid, 200 mg, 50%, from the title compound from step 1 and Intermediate 15, following a similar procedure to that described in Example 29 and 30, step 2. LCMS m/z=732 [M+H]+

Step 3: Synthesis of (S)-2-amino-4-(5-chloro-1-(7,7-dimethyl-4-((S)-1-((S)-1-methylpyrrolidin-2-yl)ethoxy)-5,7-dihydrofuro[3,4-d]pyrimidin-2-yl)-3-methyl-1H-indazol-4-yl)-7-fluorobenzo[b]thiophene-3-carbonitrile and (R)-2-amino-4-(5-chloro-1-(7,7-dimethyl-4-((S)-1-((S)-1-methylpyrrolidin-2-yl)ethoxy)-5,7-dihydrofuro[3,4-d]pyrimidin-2-yl)-3-methyl-1H-indazol-4-yl)-7-fluorobenzo[b]thiophene-3-carbonitrile

To a stirred solution of the title compound from step 2 (300 mg, 409 μmol) in DCM (9 mL) was added TFA (3 mL) and the reaction mixture was stirred for 1 h at rt. The mixture was concentrated, and the residue was purified by prep-HPLC (Method A2, Gradient: 15% B to 45% B in 15 min), to give Peak 1, Example 66, 45.1 mg, 17.4% as a white solid and Peak 2, Example 67, 20.7 mg, 22.2% as a white solid.

Example 66: LCMS: m/z=632 [M+H]⁺, 1H NMR (400 MHz, DMSO-d₆) δ 8.68 (d, 1H), 8.06 (s, 2H), 7.73 (d, 1H), 7.25 (dd, 1H), 7.20-7.09 (m, 1H), 5.52-5.41 (m, 1H), 5.02-4.87 (m, 2H), 3.03-2.93 (m, 1H), 2.74-2.63 (m, 1H), 2.41 (s, 3H), 2.27-2.15 (m, 1H), 1.91-1.80 (m, 1H), 1.77 (s, 3H), 1.69-1.63 (m, 3H), 1.50 (d, 6H), 1.36 (d, 3H).

Example 67: LCMS: m/z=632 [M+H]⁺, 1H NMR (400 MHz, DMSO) & 8.69 (d, 1H), 8.06 (s, 2H), 7.74 (d, 1H), 7.39-7.24 (m, 1H), 7.18-6.96 (m, 1H), 5.51-5.39 (m, 1H), 5.03-4.88 (m, 2H), 3.02 (s, 1H), 2.80 (s, 1H), 2.46 (s, 3H), 2.30 (s, 1H), 1.97-1.84 (m, 1H), 1.76 (s, 3H), 1.75-1.66 (m, 3H), 1.51 (d, 6H), 1.39 (d, 3H).

### Example 68

### [(S)-2-amino-4-(5-chloro-1-((S)-8-methoxy-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (R)-2-amino-4-(5-chloro-1-((S)-8-methoxy-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile] or [(S)-2-amino-4-(5-chloro-1-((R)-8-methoxy-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (R)-2-amino-4-(5-chloro-1-((R)-8-methoxy-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile]

### And Example 69

### [(R)-2-amino-4-(5-chloro-1-((S)-8-methoxy-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (S)-2-amino-4-(5-chloro-1-((S)-8-methoxy-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile] or [(R)-2-amino-4-(5-chloro-1-((R)-8-methoxy-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (S)-2-amino-4-(5-chloro-1-((R)-8-methoxy-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile]

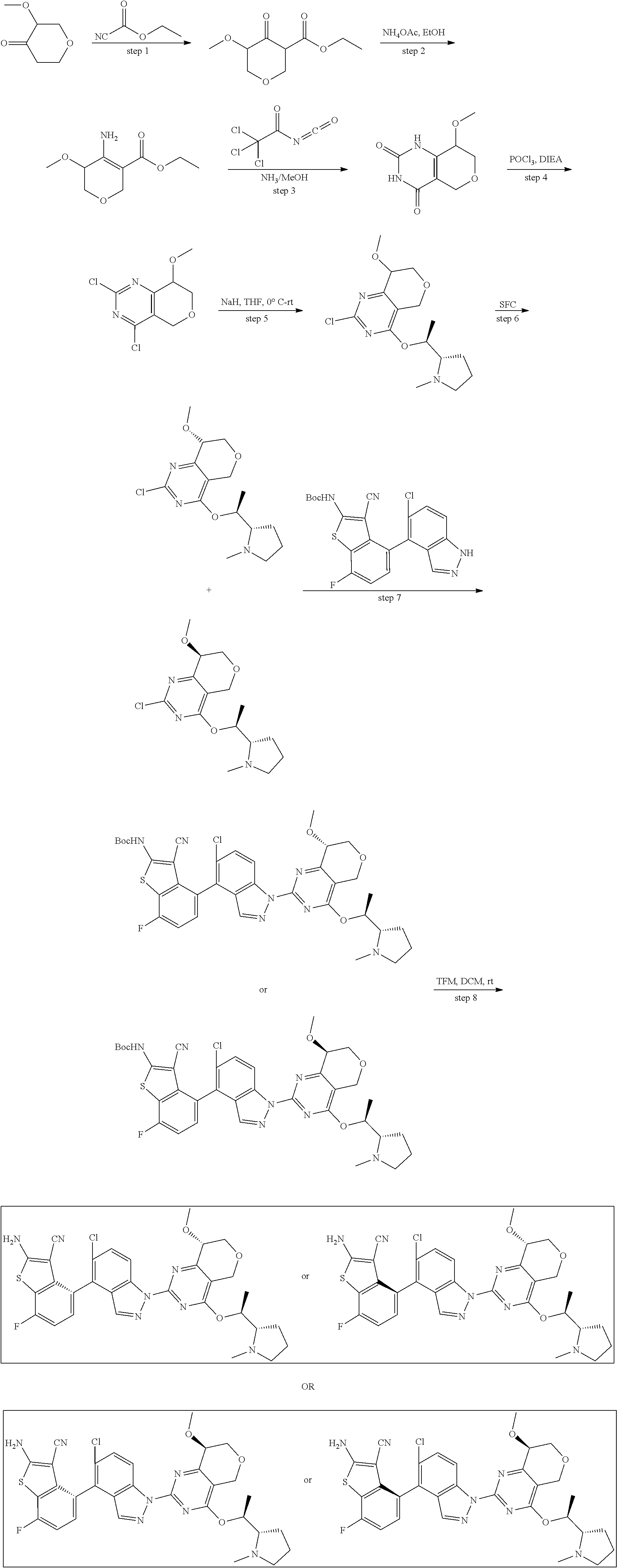

Step 1: Synthesis of ethyl 5-methoxy-4-oxotetrahydro-2H-pyran-3-carboxylate

The title compound was obtained as a white solid, 1.8 g, 38.7%, from 3-methoxyoxan-4-one and ethyl carbonocyanidate, following a similar procedure to that described in Intermediate 5A and B, step 1. LCMS: m/z=203 [M+H]⁺.

Step 2: Synthesis of ethyl 4-amino-5-methoxy-5,6-dihydro-2H-pyran-3-carboxylate

The title compound was obtained as a white solid, 1 g, 55.8%, from the title compound of step 1, following a similar procedure to that described in Intermediate 5A and B, step 2. LCMS m/z=202 [M+H]+

Step 3: Synthesis of 8-methoxy-1,5,7,8-tetrahydro-2H-pyrano[4,3-d]pyrimidine-2,4(3H)-dione

To a stirred solution of the title compound of step 2 (1 g, 4.96 mol) and trichloroacetyl isocyanate (1.86 g, 9.92 mol) in MeCN (10 mL) was stirred at 0° C. for 4 h under N₂. The mixture was filtered and the solid was dissolved in 7N NH₃ in MeOH and the mixture was stirred at 80° C. for 6 h. The cooled mixture was filtered, washing through with water to give the title compound (700 mg, 71.2%) as a yellow solid. LCMS: m/z=199 [M+H]⁺.

Step 4: Synthesis of 2,4-dichloro-8-methoxy-7,8-dihydro-5H-pyrano[4,3-d]pyrimidine:

The title compound was obtained as an off-white solid, 1 g, 42%, from the title compound of step 3, following a similar procedure to that described in Intermediate 5A and B, step 4. LCMS: m/z=235 [M+H]⁺.

Step 5: Synthesis of 2-chloro-8-methoxy-4-((S)-1-((S)-1-methylpyrrolidin-2-yl) ethoxy)-7,8-dihydro-5H-pyrano[4,3-d]pyrimidine:

The title compound was obtained as a white solid, 40 mg, 11.9%, from the title compound of step 4, following a similar procedure to that described in Example 33 and 34, step 1. LCMS: m/z=328 [M+H]⁺.

Step 6: Synthesis of (S)-2-chloro-8-methoxy-4-((S)-1-((S)-1-methylpyrrolidin-2-yl) ethoxy)-7,8-dihydro-5H-pyrano[4,3-d]pyrimidine and (R)-2-chloro-8-methoxy-4-((S)-1-((S)-1-methylpyrrolidin-2-yl)ethoxy)-7,8-dihydro-5H-pyrano[4,3-d]pyrimidine

The title compound from step 5 was purified by Prep-HPLC (Column: YMC-Actus Triart C18 ExRS, 30*150 mm, 5 μm; Mobile Phase A: Water (10 mmol/LNH₄HCO₃+0.1% NH₃·H₂O), Mobile Phase B: MeCN; Flow rate: 60 mL/min; Gradient: 27% B to 60% B in 7 min, to give Peak 1, (S)-2-chloro-8-methoxy-4-((S)-1-((S)-1-methylpyrrolidin-2-yl)ethoxy)-7,8-dihydro-5H-pyrano[4,3-d]pyrimidine or (R)-2-chloro-8-methoxy-4-((S)-1-((S)-1-methylpyrrolidin-2-yl)ethoxy)-7,8-dihydro-5H-pyrano[4,3-d]pyrimidine (20 mg, 10.4%) a white solid and Peak 2, (R)-2-chloro-8-methoxy-4-((S)-1-((S)-1-methylpyrrolidin-2-yl)ethoxy)-7,8-dihydro-5H-pyrano[4,3-d]pyrimidine or (S)-2-chloro-8-methoxy-4-((S)-1-((S)-1-methylpyrrolidin-2-yl)ethoxy)-7,8-dihydro-5H-pyrano[4,3-d]pyrimidine (20 mg, 10.4%) a white solid.

Step 7: Synthesis of tert-butyl (4-(5-chloro-1-((S)-8-methoxy-4-((S)-1-((S)-1-methylpyrrolidin-2-yl)ethoxy)-7,8-dihydro-5H-pyrano[4,3-d]pyrimidin-2-yl)-1H-indazol-4-yl)-3-cyano-7-fluorobenzo[b]thiophen-2-yl)carbamate or tert-butyl (4-(5-chloro-1-((R)-8-methoxy-4-((S)-1-((S)-1-methylpyrrolidin-2-yl)ethoxy)-7,8-dihydro-5H-pyrano[4,3-d]pyrimidin-2-yl)-1H-indazol-4-yl)-3-cyano-7-fluorobenzo[b]thiophen-2-yl)carbamate

The title compound was obtained as a white solid, 20 mg, 44.7%, from the title compound from Peak 1, step 6 and Intermediate 16, following a similar procedure to that described in Example 29 and 30, step 2. LCMS m/z=734 [M+H]+

Step 8: Synthesis of [(S)-2-amino-4-(5-chloro-1-((S)-8-methoxy-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile and (R)-2-amino-4-(5-chloro-1-((S)-8-methoxy-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile] or [(S)-2-amino-4-(5-chloro-1-((R)-8-methoxy-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile and (R)-2-amino-4-(5-chloro-1-((R)-8-methoxy-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile]

The title compounds were obtained from the title compound from step 7, following a similar procedure to that described in Example 66 and 67, step 3.

Example 68, 2.1 mg, 12.2% as a white solid. LCMS: m/z=634 [M+H]⁺, 1H NMR (400 MHz, DMSO-d₆) δ 8.71 (d, 1H), 8.04 (s, 2H), 7.95 (s, 1H), 7.76 (d, 1H), 7.29 (t, 1H), 7.18 (t, 1H), 5.45 (q, 1H), 4.70 (d, 1H), 4.48 (t, 1H), 4.24 (s, 1H), 4.17 (d, 1H), 3.91 (d, 1H), 3.60 (s, 3H), 2.96 (s, 1H), 2.64 (d, 1H), 2.36 (s, 3H), 2.21 (s, 1H), 1.88-1.83 (m, 1H), 1.75-1.62 (m, 3H), 1.35 (d, 3H).

Example 69, 1.8 mg, 10.4%, as a white solid. LCMS: m/z=634 [M+H]⁺, 1H NMR (400 MHz, DMSO-d₆) δ 8.71 (d, 1H), 8.04 (s, 2H), 7.95 (s, 1H), 7.76 (d, 1H), 7.29 (t, 1H), 7.18 (t, 1H), 5.45 (q, 1H), 4.70 (d, 1H), 4.48 (d, 1H), 4.24 (s, 1H), 4.17 (d, 1H), 3.91 (d, 1H), 2.96 (s, 1H), 2.64 (d, 1H), 2.36 (s, 3H), 2.21 (d, 1H), 1.87-1.82 (m, 1H), 1.76-1.64 (m, 3H), 1.35 (d, 3H).

### Example 70

### [(S)-2-amino-4-(5-chloro-1-((R)-8-methoxy-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (R)-2-amino-4-(5-chloro-1-((R)-8-methoxy-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile] or [(S)-2-amino-4-(5-chloro-1-((S)-8-methoxy-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (R)-2-amino-4-(5-chloro-1-((S)-8-methoxy-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile]

### And Example 71

### [(R)-2-amino-4-(5-chloro-1-((R)-8-methoxy-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (S)-2-amino-4-(5-chloro-1-((R)-8-methoxy-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile] or [(R)-2-amino-4-(5-chloro-1-((S)-8-methoxy-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (S)-2-amino-4-(5-chloro-1-((S)-8-methoxy-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile]

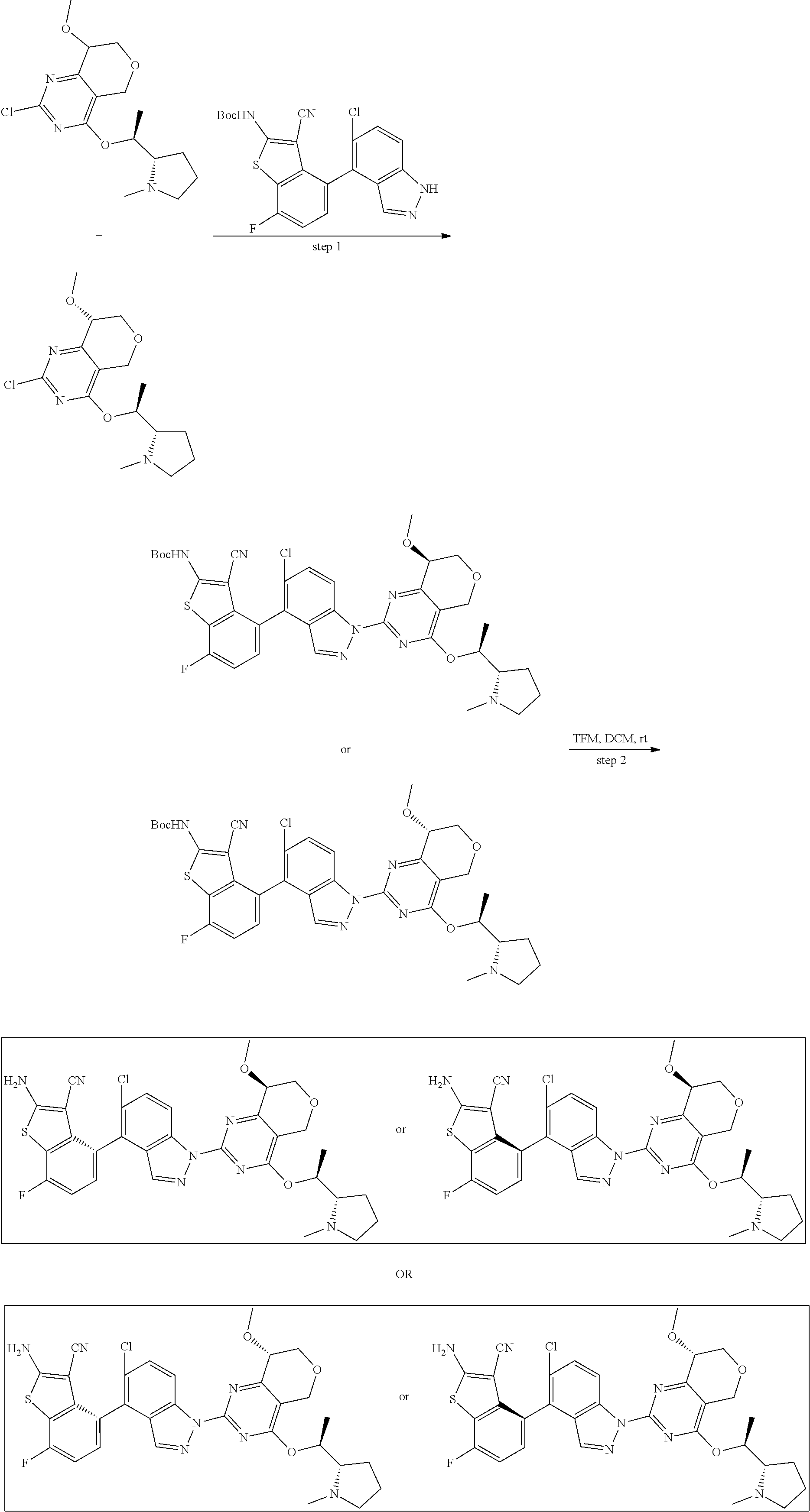

Step 1: Synthesis of tert-butyl (4-(5-chloro-1-((R)-8-methoxy-4-((S)-1-((S)-1-methylpyrrolidin-2-yl)ethoxy)-7,8-dihydro-5H-pyrano[4,3-d]pyrimidin-2-yl)-1H-indazol-4-yl)-3-cyano-7-fluorobenzo[b]thiophen-2-yl)carbamate or tert-butyl (4-(5-chloro-1-((S)-8-methoxy-4-((S)-1-((S)-1-methylpyrrolidin-2-yl)ethoxy)-7,8-dihydro-5H-pyrano[4,3-d]pyrimidin-2-yl)-1H-indazol-4-yl)-3-cyano-7-fluorobenzo[b]thiophen-2-yl)carbamate

The title compound was obtained as a white solid, 20 mg, 44.7%, from the title compound from Peak 2, step 6 (Example 68 and 69) and Intermediate 16, following a similar procedure to that described in Example 29 and 30, step 2. LCMS m/z=734 [M+H]+

Step 2: Synthesis of [(S)-2-amino-4-(5-chloro-1-((R)-8-methoxy-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile and (R)-2-amino-4-(5-chloro-1-((R)-8-methoxy-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile] or [(S)-2-amino-4-(5-chloro-1-((S)-8-methoxy-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile and (R)-2-amino-4-(5-chloro-1-((S)-8-methoxy-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile]

The title compounds were obtained from the title compound from step 1, following a similar procedure to that described in Example 66 and 67, step 3.

Example 70, 2.1 mg 12.2% as a white solid. LCMS: m/z=634 [M+H]⁺, 1H NMR (400 MHz, DMSO-d₆) δ 8.70 (d, 1H), 8.03 (s, 2H), 7.95 (s, 1H), 7.75 (d, 1H), 7.29 (dd, 1H), 7.18 (t, 1H), 5.46 (p, 1H), 4.66 (d, 1H), 4.52 (d, 1H), 4.23 (q, 1H), 4.17 (dd, 1H), 3.90 (dd, 1H), 3.60 (s, 3H), 3.02-2.93 (m, 1H), 2.66 (dt, 1H), 2.46 (s, 1H), 2.38 (s, 3H), 2.28-2.17 (m, 1H), 1.91-1.82 (m, 1H), 1.78-1.60 (m, 3H), 1.33 (d, 3H).

Example 71, (1.8 mg, 10.4%) as a white solid. LCMS: m/z=634 [M+H]⁺, 1H NMR (400 MHz, DMSO-d₆) δ 8.71 (d, 1H), 8.04 (s, 2H), 7.95 (s, 1H), 7.76 (d, 1H), 7.29 (dd, 1H), 7.18 (t, 1H), 5.52-5.44 (m, 1H), 4.66 (d, 1H), 4.52 (d, 1H), 4.23 (s, 1H), 4.21-4.13 (m, 1H), 3.90 (d, 1H), 3.60 (s, 3H), 2.96 (d, 1H), 2.38 (s, 3H), 2.22 (q, 1H), 1.93-1.79 (m, 1H), 1.75-1.67 (m, 3H), 1.34 (d, 3H).

### Example 72

### (R)-2-amino-4-(5-chloro-3-methyl-1-(4-((S)-1-((S)-1-methylpyrrolidin-2-yl)ethoxy)-5,6,7,8-tetrahydroquinazolin-2-yl)-1H-indazol-4-yl)-7-fluorobenzo[b]thiophene-3-carbonitrile or (S)-2-amino-4-(5-chloro-3-methyl-1-(4-((S)-1-((S)-1-methylpyrrolidin-2-yl)ethoxy)-5,6,7,8-tetrahydroquinazolin-2-yl)-1H-indazol-4-yl)-7-fluorobenzo[b]thiophene-3-carbonitrile

### And Example 73

### (S)-2-amino-4-(5-chloro-3-methyl-1-(4-((S)-1-((S)-1-methylpyrrolidin-2-yl)ethoxy)-5,6,7,8-tetrahydroquinazolin-2-yl)-1H-indazol-4-yl)-7-fluorobenzo[b]thiophene-3-carbonitrile or (R)-2-amino-4-(5-chloro-3-methyl-1-(4-((S)-1-((S)-1-methylpyrrolidin-2-yl)ethoxy)-5,6,7,8-tetrahydroquinazolin-2-yl)-1H-indazol-4-yl)-7-fluorobenzo[b]thiophene-3-carbonitrile

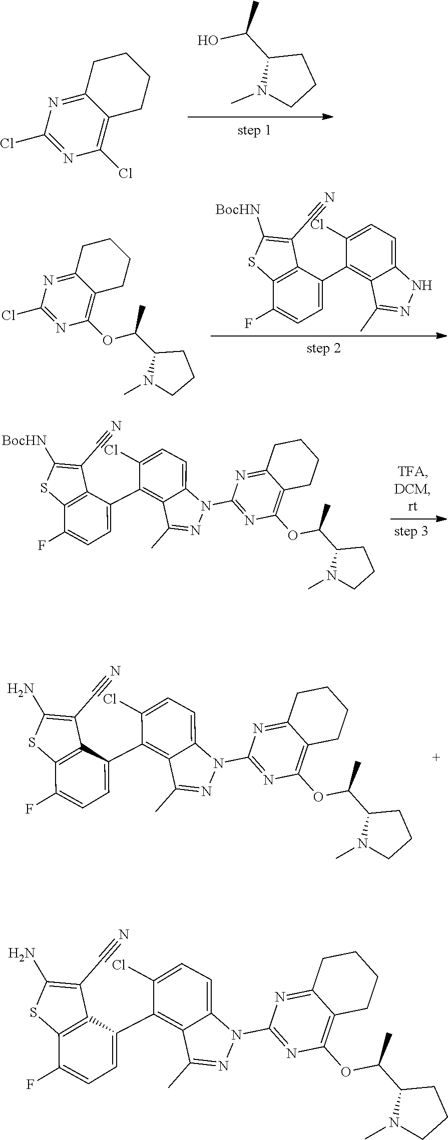

Step 1: Synthesis of 2-chloro-4-((S)-1-((S)-1-methylpyrrolidin-2-yl)ethoxy)-5,6,7,8-tetrahydroquinazoline

The title compound was obtained as a yellow solid, 300 mg, 41.2%, from(S)-1-((S)-1-methylpyrrolidin-2-yl)ethan-1-ol and 2,4-dichloro-5,6,7,8-tetrahydroquinazoline, following a similar procedure to that described in Example 47 and 48, step 3. LCMS: m/z=296 [M+H]⁺.

Step 2: Synthesis of tert-butyl (4-(5-chloro-3-methyl-1-(4-((S)-1-((S)-1-methylpyrrolidin-2-yl)ethoxy)-5,6,7,8-tetrahydroquinazolin-2-yl)-1H-indazol-4-yl)-3-cyano-7-fluorobenzo[b]thiophen-2-yl)carbamate

The title compound was obtained as a white solid, 140 mg, 27.6%, from the title compound of step 1 and Intermediate 15 following a similar procedure to that described in Example 29 and 30, step 2. LCMS: m/z=716 [M+H]⁺.

Step 3: Synthesis of (R)-2-amino-4-(5-chloro-3-methyl-1-(4-((S)-1-((S)-1-methylpyrrolidin-2-yl)ethoxy)-5,6,7,8-tetrahydroquinazolin-2-yl)-1H-indazol-4-yl)-7-fluorobenzo[b]thiophene-3-carbonitrile and (S)-2-amino-4-(5-chloro-3-methyl-1-(4-((S)-1-((S)-1-methylpyrrolidin-2-yl)ethoxy)-5,6,7,8-tetrahydroquinazolin-2-yl)-1H-indazol-4-yl)-7-fluorobenzo[b]thiophene-3-carbonitrile

The title compounds were obtained from the title compound of step 2, following a similar procedure to that described in Example 66 and 67, step 3.

Peak 1, Example 72 (36.1 mg, 38%, as a white solid). LCMS: m/z=616 [M+H]⁺, 1H NMR (400 MHz, DMSO-d₆) δ 8.67 (d, 1H), 8.04 (s, 2H), 7.68 (d, 1H), 7.25 (dd, 1H), 7.16 (t, 1H), 5.43 (q, 1H), 2.99 (q, 1H), 2.81 (d, 2H), 2.71-2.61 (m, 1H), 2.54 (s, 1H), 2.40 (s, 3H), 2.22 (q, 1H), 1.88-1.65 (m, 11H), 1.33 (d, 3H).

Peak 2, Example 73 (45.6 mg, 38.9%) as a white solid. LCMS: m/z=616 [M+H]⁺, 1H NMR (400 MHz, DMSO-d₆) δ 8.68 (d, 1H), 8.05 (s, 2H), 7.67 (d, 1H), 7.27 (dd, 1H), 7.16 (t, 1H), 5.45 (q, 1H), 2.99 (s, 1H), 2.81 (d, 2H), 2.68 (q, 1H), 2.54 (s, 1H), 2.41 (s, 3H), 2.22 (q, 1H), 1.84-1.66 (m, 11H), 1.33 (d, 3H).

### Example 74

### 2-amino-7-fluoro-4-(1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]selenophene-3-carbonitrile

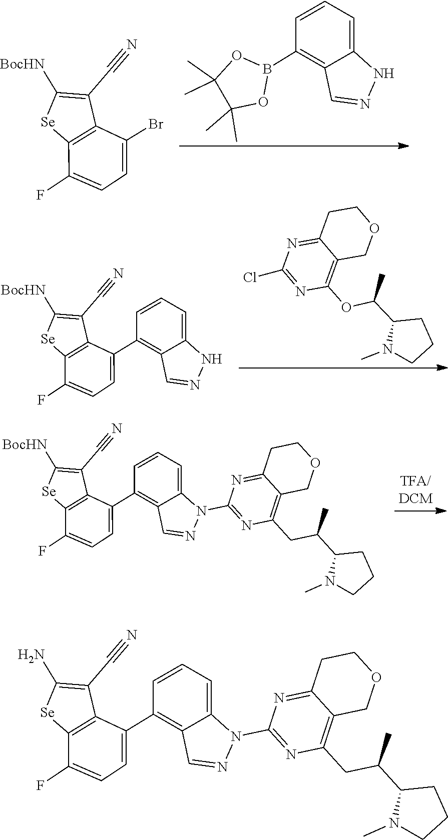 [SMILES: CC(C)(C)OC(=O)Nc1[se]c2c(F)ccc(Br)c2c1C#N ; CC1(C)OB(c2cccc3[nH]ncc23)OC1(C)C ; CC(C)(C)OC(=O)Nc1[se]c2c(F)ccc(-c3cccc4[nH]ncc34)c2c1C#N ; C[C@H](Oc1nc(Cl)nc2c1COCC2)[C@@H]1CCCN1C ; C[C@H](Cc1nc(-n2ncc3c(-c4ccc(F)c5[se]c(NC(=O)OC(C)(C)C)c(C#N)c45)cccc32)nc2c1COCC2)[C@@H]1CCCN1C ; C[C@H](Cc1nc(-n2ncc3c(-c4ccc(F)c5[se]c(N)c(C#N)c45)cccc32)nc2c1COCC2)[C@@H]1CCCN1C]

Step 1: Synthesis of tert-butyl (3-cyano-7-fluoro-4-(1H-indazol-4-yl)benzo[b]selenophen-2-yl)carbamate

To a solution of Intermediate 20 (200 mg, 478 μmol) and 4-(4,4,5,5-tetramethyl-1,3,2-dioxaborolan-2-yl)-1H-indazole (139 mg, 573 μmol) in dioxane/H₂O (4 mL/1 mL) were added K₂CO₃ (130 mg, 956 μmol) and Pd(dtbpf)Cl₂ (31.1 mg, 47.8 μmol) under N₂ and the reaction was stirred for 2 h at 80° C. Water (10 mL) was added and the mixture was extracted with DCM (10 mL×3). The combined organic layers were dried over Na₂SO₄ and concentrated. The crude product was purified by Prep-TLC with DCM:MeOH=10:1 to give the title compound (170 mg; 78%) as a yellow solid. LCMS: m/z=456 [M+H]⁺.

Step 2: Synthesis of tert-butyl (3-cyano-7-fluoro-4-(1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]selenophen-2-yl)carbamate

The title compound was obtained as a yellow solid, 120 mg, 51%, from the title compound from step 1 and Intermediate 1, following a similar procedure to that described in Example 29 and 30, step 2. LCMS m/z=718 [M+H]+

Step 3: Synthesis of 2-amino-7-fluoro-4-(1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]selenophene-3-carbonitrile

The title compound was obtained as a white solid, 23.7 mg, 34%, from the title compound from step 2, following a similar procedure to that described in Example 38 and 39, step 2 and step 3. LCMS: m/z=618 [M+H]⁺, 1H NMR (400 MHz, DMSO-d₆) δ 8.69 (dd, 1H), 7.99 (s, 2H), 7.97 (s, 1H), 7.61 (dd, 1H), 7.32-7.23 (m, 2H), 7.07 (t, 1H), 5.48 (p, 1H), 4.67-4.53 (m, 2H), 4.01 (t, 2H), 3.02-2.95 (m, 1H), 2.91 (q, 2H), 2.65 (q, 1H), 2.38 (s, 3H), 2.23-2.21 (m, 1H), 1.93-1.78 (m, 1H), 1.79-1.64 (m, 3H), 1.34 (d, 3H).

### Example 75

### (S)-2-amino-4-(3,5-difluoro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (R)-2-amino-4-(3,5-difluoro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

### And Example 76

### (R)-2-amino-4-(3,5-difluoro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (S)-2-amino-4-(3,5-difluoro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

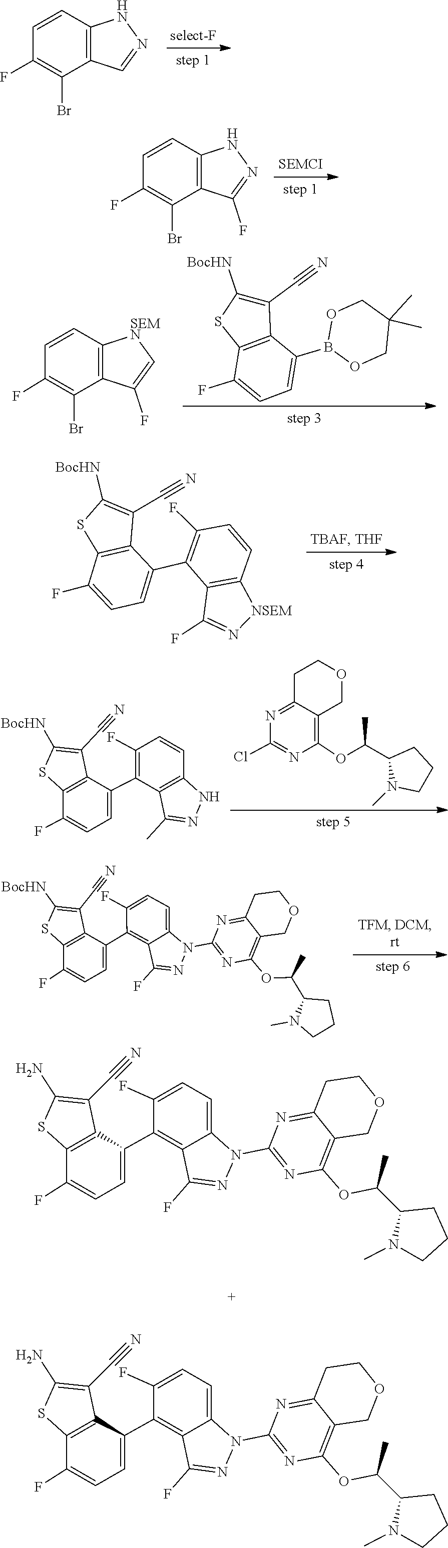

Step 1: Synthesis of 4-bromo-3,5-difluoro-1H-indazole

A solution of 4-bromo-5-fluoro-1H-indazole (2 g, 9.30 mmol) and Selectfluor (4.92 g, 13.9 mmol) in MeCN (20 mL) was stirred at 80° C. for 6 h under N₂. The mixture was diluted with EtOAc (100 mL) and washed with brine (50 mL). The organic layer was dried with Na₂SO₄ and concentrated in vacuo. The residue was purified by silica gel column chromatography (DCM:MeOH=20:1 to 10:1) to give the title compound, 700 mg, 32.4%, as a white solid. LCMS m/z=233 [M+H]+

Step 2: Synthesis of 4-bromo-3,5-difluoro-1-((2-(trimethylsilyl)ethoxy)methyl)-1H-indazole

The title compound from step 1 (680 mg, 2.91 mmol) was suspended in THF (4 mL), NaH (60% in mineral oil, 348 mg, 8.73 mmol) was added and the mixture was stirred for 30 min at 0° C. A solution of SEMCl (726 mg, 4.36 mmol) in THF (4 mL) was added and the reaction mixture was stirred for 4 h at 0° C. The resulting solution was poured into water, the mixture was extracted with EtOAc (30 mL×3) and the combined organic extracts washed with water (20 mL) and brine (10 mL). The organic layer was dried with Na₂SO₄ and concentrated in vacuo. The residue was purified by prep-TLC (EtOAc:PE=2:1) to give the title compound, 250 mg (39.4%) as a white solid. LCMS m/z=363 [M+H]+

Step 3: Synthesis of tert-butyl (3-cyano-4-(3,5-difluoro-1-((2-(trimethylsilyl)ethoxy)methyl)-1H-indazol-4-yl)-7-fluorobenzo[b]thiophen-2-yl)carbamate

A solution of the title compound from step 2 (240 mg, 1.10 mmol), Intermediate 9 (533 mg, 1.32 mmol), DePhosPdCl₂ (59.2 mg, 110 μmol) and Cs₂CO₃ (716 mg, 2.20 mmol) in dioxane (3 mL) was stirred at 80° C. for 4 h under N₂. The mixture was diluted with EtOAc (50 mL) and washed with brine (20 mL). The organic layer was dried with Na₂SO₄ and concentrated in vacuo. The residue was purified by prep-TLC (EtOAc:PE=2:1) to give the title compound, 170 mg, 49.1% as a white solid. LCMS m/z=575 [M+H]+

Step 4: Synthesis of tert-butyl (3-cyano-4-(3,5-difluoro-1H-indazol-4-yl)-7-fluorobenzo[b]thiophen-2-yl)carbamate

To a stirred solution of the title compound from step 3 (170 mg, 542 μmol) in THF (2 mL) was added TBAF (171 mg, 542 μmol, 1M solution in THF) and the reaction mixture was stirred at 80° C. for 4 h under N₂. The mixture was diluted with EtOAc (40 mL) and washed with brine (10 mL). The organic layer was dried with Na₂SO₄ and concentrated in vacuo. The residue was purified by prep-TLC (DCM:MeOH=20:1) to give the title compound, 80 mg, 33.3%, as a yellow solid. LCMS m/z=445 [M+H]+

Step 5: Synthesis of tert-butyl (3-cyano-4-(3,5-difluoro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophen-2-yl)carbamate

A solution of the title compound of step 4 (80 mg, 180 μmol), Intermediate 1 (64.0 mg, 215 μmol), GPhos Pd G3 (16.3 mg, 18.0 μmol) and Cs₂CO₃ (117 mg, 360 μmol) in dioxane (3 mL) was stirred at 100° C. for 4 h under N₂. The cooled mixture was diluted with DCM (40 mL), washed with brine (20 mL), the organic layer was dried with Na₂SO₄ and concentrated in vacuo. The crude product was purified by prep-TLC (DCM:MeOH=20:1) to give the title compound, 70 mg, 55.1%, as a white solid. LCMS m/z=706 [M+H]+

Step 6: Synthesis of (S)-2-amino-4-(3,5-difluoro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile and (R)-2-amino-4-(3,5-difluoro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

To a stirred solution of the title compound from step 5 (65 mg, 92.0 μmol) in DCM (1.5 mL) was added TFA (0.5 mL) in portions at rt under N₂ and the reaction mixture was stirred for 0.5 h. The reaction mixture was evaporated under reduced pressure and the crude product was purified by Prep-HPLC (Method A2, Gradient: 10% B to 42% B in 15 min) to give Peak 1, Example 75 (13.5 mg, 24.2%) as a white solid, and Peak 2, Example 76, (12.4 mg, 22.2%) as a white solid.

Example 75. LCMS: m/z=606 [M+H]⁺, 1H NMR (400 MHz, DMSO-d₆) δ 8.75 (ddd, 1H), 8.11 (s, 2H), 7.70 (t, 1H), 7.40 (dd, 1H), 7.16 (dd, 1H), 5.40 (p, 1H), 4.66-4.52 (m, 2H), 4.00 (t, 2H), 3.03-2.98 (m, 1H), 2.91 (t, 2H), 2.74-2.68 (m, 1H), 2.41 (s, 3H), 2.31-2.25 (m, 1H), 1.92-1.83 (m, 1H), 1.78-1.67 (m, 3H), 1.34 (d, 3H).

Example 76, LCMS: m/z=606 [M+H]⁺, 1H NMR (400 MHz, DMSO-d₆) δ 8.75 (ddd, 1H), 8.11 (s, 2H), 7.69 (t, 1H), 7.41 (dd, 1H), 7.17 (t, 1H), 5.42 (p, 1H), 4.65-4.51 (m, 2H), 4.00 (t, 2H), 3.02-2.97 (m, 1H), 2.95-2.87 (m, 2H), 2.72-2.68 (m, 1H), 2.40 (s, 3H), 2.26 (td, 1H), 1.93-1.62 (m, 4H), 1.34 (d, 3H).

### Example 77

### (S)-2-amino-4-(5-chloro-3-methyl-1-(4-((S)-1-((S)-1-methylpyrrolidin-2-yl)ethoxy)-5,8-dihydro-6H-pyrano[3,4-d]pyrimidin-2-yl)-1H-indazol-4-yl)-7-fluorobenzo[b]thiophene-3-carbonitrile or (R)-2-amino-4-(5-chloro-3-methyl-1-(4-((S)-1-((S)-1-methylpyrrolidin-2-yl)ethoxy)-5,8-dihydro-6H-pyrano[3,4-d]pyrimidin-2-yl)-1H-indazol-4-yl)-7-fluorobenzo[b]thiophene-3-carbonitrile

### And Example 78

### (R)-2-amino-4-(5-chloro-3-methyl-1-(4-((S)-1-((S)-1-methylpyrrolidin-2-yl)ethoxy)-5,8-dihydro-6H-pyrano[3,4-d]pyrimidin-2-yl)-1H-indazol-4-yl)-7-fluorobenzo[b]thiophene-3-carbonitrile or (S)-2-amino-4-(5-chloro-3-methyl-1-(4-((S)-1-((S)-1-methylpyrrolidin-2-yl)ethoxy)-5,8-dihydro-6H-pyrano[3,4-d]pyrimidin-2-yl)-1H-indazol-4-yl)-7-fluorobenzo[b]thiophene-3-carbonitrile

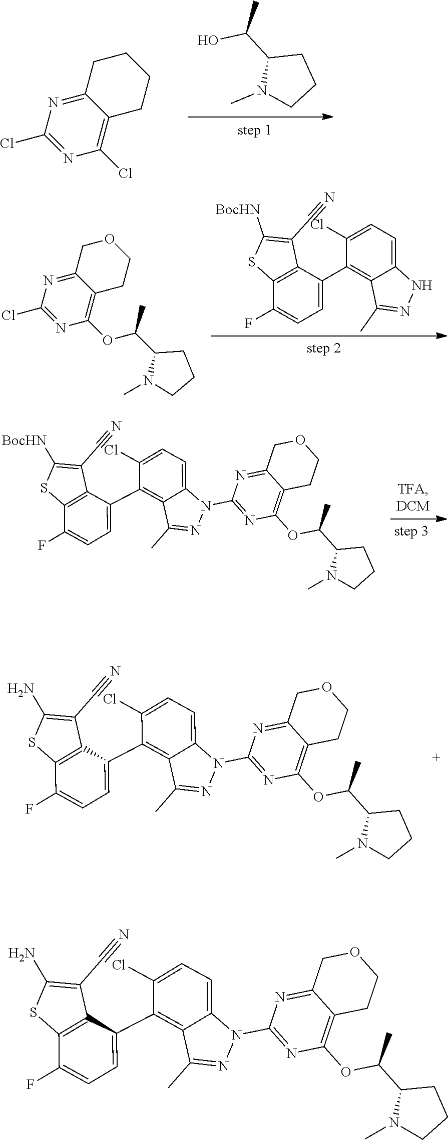

Step 1: Synthesis of 2-chloro-4-((S)-1-((S)-1-methylpyrrolidin-2-yl)ethoxy)-5,8-dihydro-6H-pyrano[3,4-d]pyrimidine

The title compound was obtained as a white solid, 140 mg, 24%, from(S)-1-((S)-1-methylpyrrolidin-2-yl)ethan-1-ol and 2,4-dichloro-5,8-dihydro-6H-pyrano[3,4-d]pyrimidine, following a similar procedure to that described in Example 47 and 48, step 3. LCMS m/z=298 [M+H]+

Step 2: Synthesis of tert-butyl (4-(5-chloro-3-methyl-1-(4-((S)-1-((S)-1-methylpyrrolidin-2-yl)ethoxy)-5,8-dihydro-6H-pyrano[3,4-d]pyrimidin-2-yl)-1H-indazol-4-yl)-3-cyano-7-fluorobenzo[b]thiophen-2-yl)carbamate

A mixture of Intermediate 15 (130 mg, 284 μmol), the title compound from step 1 (126 mg, 425 μmol), Gphos Pd G6 TES (26.8 mg, 28.4 μmol) and Cs₂CO₃ (279 mg, 851 μmol) in dioxane (20 mL) was stirred at 100° C. for 3 h. The mixture was diluted with water (50 mL) and extracted with EtOAc (100 mL×2). The combined organic layer was washed with brine (50 mL×2), dried over Na₂SO₄ and concentrated in vacuo. The residue was purified by prep-TLC with DCM/MeOH (10:1) to afford the title compound (220 mg, 97.5%) as a white solid. LCMS m/z=718 [M+H]+

Step 3: Synthesis of (S)-2-amino-4-(5-chloro-3-methyl-1-(4-((S)-1-((S)-1-methylpyrrolidin-2-yl)ethoxy)-5,8-dihydro-6H-pyrano[3,4-d]pyrimidin-2-yl)-1H-indazol-4-yl)-7-fluorobenzo[b]thiophene-3-carbonitrile and (R)-2-amino-4-(5-chloro-3-methyl-1-(4-((S)-1-((S)-1-methylpyrrolidin-2-yl)ethoxy)-5,8-dihydro-6H-pyrano[3,4-d]pyrimidin-2-yl)-1H-indazol-4-yl)-7-fluorobenzo[b]thiophene-3-carbonitrile

The title compounds were obtained from the title compound of step 2, following a similar procedure to that described in Example 75 and 76, step 6.

Peak 1, Example 77:20.9 mg, 17.5% as a white solid. LCMS: m/z=618 [M+H]⁺; 1H NMR (400 MHz, MeOD) δ 8.74 (d, 1H), 7.67 (d, 1H), 7.22 (dd, 1H), 7.07 (dd, 1H), 5.66-5.48 (m, 1H), 4.76-4.69 (m, 2H), 4.10-3.95 (m, 2H), 3.42 (s, 2H), 2.94-2.63 (m, 6H), 2.24 (s, 1H), 2.07-1.90 (m, 3H), 1.87 (s, 3H), 1.52 (d, 3H).

and Peak 2, Example 78, 7.5 mg, 3.58% as a white solid. LCMS: m/z=618 [M+H]⁺; 1H NMR (400 MHz, DMSO-d₆) δ 8.65 (d, 1H), 8.03 (s, 2H), 7.70 (d, 1H), 7.25 (dd, 1H), 7.16 (dd, 1H), 5.53-5.41 (m, 1H), 4.69 (s, 2H), 4.04-3.85 (m, 2H), 3.08 (s, 1H), 2.63 (d, 2H), 1.91 (s, 1H), 1.75-1.71 (m, 6H), 1.39 (d, 3H).

### Example 79

### (S)-2-amino-7-fluoro-4-(3-fluoro-5-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophene-3-carbonitrile or (R)-2-amino-7-fluoro-4-(3-fluoro-5-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophene-3-carbonitrile

### And Example 80

### (R)-2-amino-7-fluoro-4-(3-fluoro-5-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophene-3-carbonitrile or (S)-2-amino-7-fluoro-4-(3-fluoro-5-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophene-3-carbonitrile

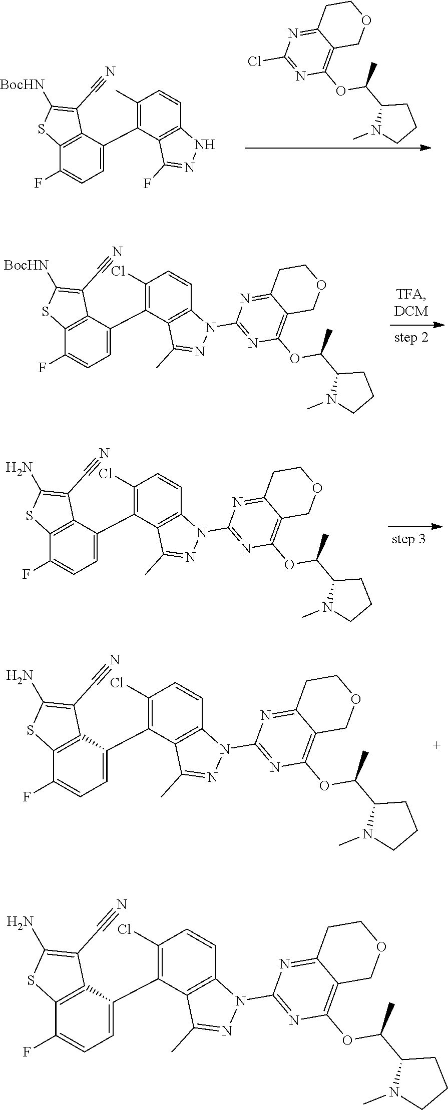

Step 1: Synthesis of tert-butyl (3-cyano-7-fluoro-4-(3-fluoro-5-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophen-2-yl)carbamate

The title compound was obtained as a brown solid, 80 mg, 25%, from Intermediate 12 and Intermediate 1, following a similar procedure to that described in Example 77 and 78, step 2. LCMS m/z=702 [M+H]+

Steps 2 and 3: Synthesis of (S)-2-amino-7-fluoro-4-(3-fluoro-5-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophene-3-carbonitrile and (R)-2-amino-7-fluoro-4-(3-fluoro-5-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophene-3-carbonitrile

The title compounds were obtained from the title compound of step 1, following a similar procedure to that described in Example 1 and 2, steps 2 and 3.

Peak 1, Example 79, (10.9 mg, 22%) as a yellow solid. LCMS: m/z=602 [M+H]⁺, 1H NMR (400 MHz, DMSO-d₆) δ 8.59 (dd, 1H), 8.03 (s, 2H), 7.62 (d, 1H), 7.24 (dd, 1H), 7.13 (t, 1H), 5.42 (p, 1H), 4.64-4.51 (m, 2H), 4.00 (t, 2H), 3.02-2.93 (m, 1H), 2.89 (t, 2H), 2.64 (dt, 1H), 2.39 (s, 3H), 2.27-2.17 (m, 1H), 2.13 (s, 3H), 1.88-1.71 (m, 4H), 1.33 (d, 3H).

and Peak 2, Example 80, (12.0 mg, 24%) as a white solid. LCMS: m/z=602 [M+H]⁺, 1H NMR (400 MHz, DMSO-d₆) δ 8.61 (dd, 1H), 8.01 (s, 2H), 7.63 (d, 1H), 7.23 (dd, 1H), 7.13 (dd, 1H), 5.44 (p, 1H), 4.64-4.50 (m, 2H), 4.00 (t, 2H), 3.01-2.93 (m, 1H), 2.89 (t, 2H), 2.67-2.59 (m, 1H), 2.38 (s, 3H), 2.21 (td, 1H), 2.13 (s, 3H), 1.90-1.66 (m, 4H), 1.33 (d, 3H).

### Example 81

### 2-amino-5,7-difluoro-4-(1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]selenophene-3-carbonitrile

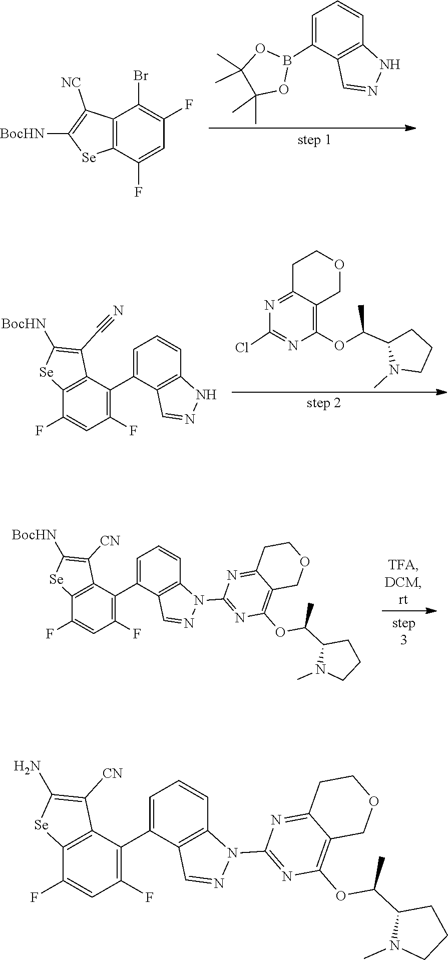

Step 1: Synthesis of tert-butyl (3-cyano-5,7-difluoro-4-(1H-indazol-4-yl)benzo[b]selenophen-2-yl)carbamate

The title compound was obtained as a yellow solid, 200 mg, 61.1%, from Intermediate 19 and 4-(4,4,5,5-tetramethyl-1,3,2-dioxaborolan-2-yl)-1H-indazole, following a similar procedure to that described in Example 74, step 1. LCMS m/z=475 [M+H]+

Step 2: Synthesis of tert-butyl (3-cyano-5,7-difluoro-4-(1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]selenophen-2-yl)carbamate:

The title compound was obtained as a white solid, 150 mg, 42.6%, from the title compound from step 1 and Intermediate 1, following a similar procedure to that described in Example 77 and 78, step 2. LCMS m/z=736 [M+H]+

Step 3: Synthesis of 2-amino-5,7-difluoro-4-(1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]selenophene-3-carbonitrile

To a stirred solution of the title compound from step 2 (150 mg, 71.5 μmol) in DCM (1.5 mL) was added TFA (0.5 mL) in portions at 25° C. under N₂ and the reaction mixture was stirred for 0.5 h. The mixture was concentrated in vacuo. The crude product was purified by Prep-HPLC: Column: YMC-Actus Triart C18 ExRS, 30*150 mm, 5 μm; Mobile Phase A: Water (10 mmol/L NH₄HCO₃+0.1% NH₃·H₂O), Mobile Phase B: MeCN; Flow rate: 60 mL/min; Gradient: 27% B to 60% B in 7 min, to give the title compound (50.2 mg, 43.0%) as an off-white solid. LCMS: m/z=636 [M+H]⁺. 1H NMR (400 MHz, DMSO-d₆) δ 8.73 (d, 1H), 8.14 (d, 2H), 8.05 (s, 1H), 7.65 (dd, 1H), 7.30 (d, 1H), 7.20 (t, 1H), 5.48 (h, 1H), 4.69-4.55 (m, 2H), 4.02 (t, 2H), 3.10 (dq, 1H), 2.94 (t, 2H), 2.89 (d, 1H), 2.43 (t, 1H), 1.98-1.90 (m, 1H), 1.84-1.69 (m, 3H), 1.38 (dd, 3H).

### Example 82

### 2-amino-4-(1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]selenophene-3-carbonitrile

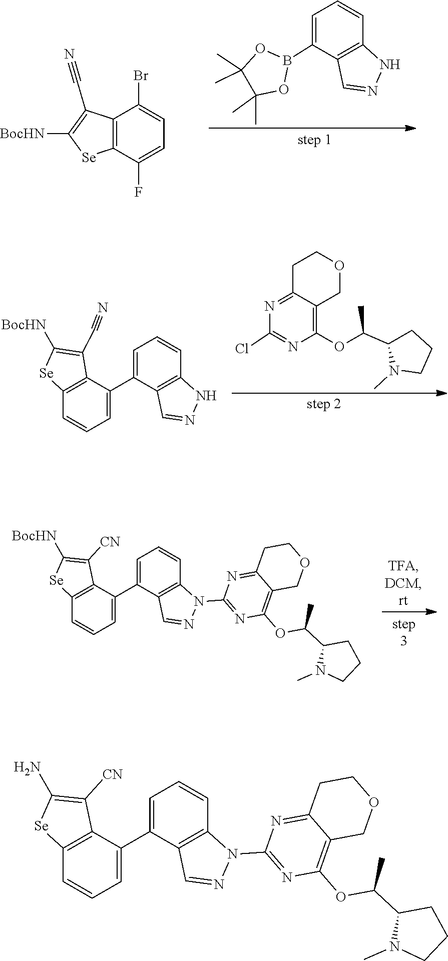 [SMILES: CC(C)(C)OC(=O)Nc1[se]c2c(F)ccc(Br)c2c1C#N ; CC1(C)OB(c2cccc3[nH]ncc23)OC1(C)C ; CC(C)(C)OC(=O)Nc1[se]c2cccc(-c3cccc4[nH]ncc34)c2c1C#N ; C[C@H](Oc1nc(Cl)nc2c1COCC2)[C@@H]1CCCN1C ; C[C@H](Oc1nc(-n2ncc3c(-c4cccc5[se]c(NC(=O)OC(C)(C)C)c(C#N)c45)cccc32)nc2c1COCC2)[C@@H]1CCCN1C ; C[C@H](Oc1nc(-n2ncc3c(-c4cccc5[se]c(N)c(C#N)c45)cccc32)nc2c1COCC2)[C@@H]1CCCN1C]

Step 1: Synthesis of tert-butyl (3-cyano-4-(1H-indazol-4-yl)benzo[b]selenophen-2-yl)carbamate

The title compound was obtained as a yellow solid, 200 mg, 61%, from Intermediate 21 and 4-(4,4,5,5-tetramethyl-1,3,2-dioxaborolan-2-yl)-1H-indazole, following a similar procedure to that described in-Example 74, step 1. LCMS: m/z=439 [M+H]⁺.

Step 2: Synthesis of tert-butyl (3-cyano-4-(1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]selenophen-2-yl)carbamate

The title compound was obtained as a white solid, 120 mg, 37.6%, from the title compound from step 1 and Intermediate 1 following a similar procedure to that described in Example 77 and 78, step 2. LCMS: m/z=700 [M+H]⁺.

Step 3: Synthesis of 2-amino-4-(1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]selenophene-3-carbonitrile

The title compound was obtained as an off-white solid, 28.3 mg, 33%, from the title compound from step 2, following a similar procedure to that described in Example 81, step 3. LCMS: m/z=600 [M+H]⁺. 1H NMR (400 MHz, DMSO-d₆) δ 8.68 (dd, 1H), 7.93 (d, 1H), 7.85 (dd, 1H), 7.74 (s, 2H), 7.61 (dd, 1H), 7.29-7.18 (m, 2H), 7.17 (t, 1H), 5.49 (dp, 1H), 4.63 (d, 1H), 4.57 (d, 1H), 4.02 (t, 2H), 2.98 (qd, 1H), 2.92 (t, 2H), 2.66 (dt, 1H), 2.54 (s, 1H), 2.39 (d, 3H), 2.28-2.17 (m, 1H), 1.93-1.78 (m, 1H), 1.76-1.65 (m, 3H), 1.34 (dd, 3H).

### Example 83

### (S)-2-amino-4-(5-chloro-3-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophene-3-carbonitrile or (R)-2-amino-4-(5-chloro-3-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophene-3-carbonitrile

### And Example 84

### (R)-2-amino-4-(5-chloro-3-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophene-3-carbonitrile or (S)-2-amino-4-(5-chloro-3-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophene-3-carbonitrile

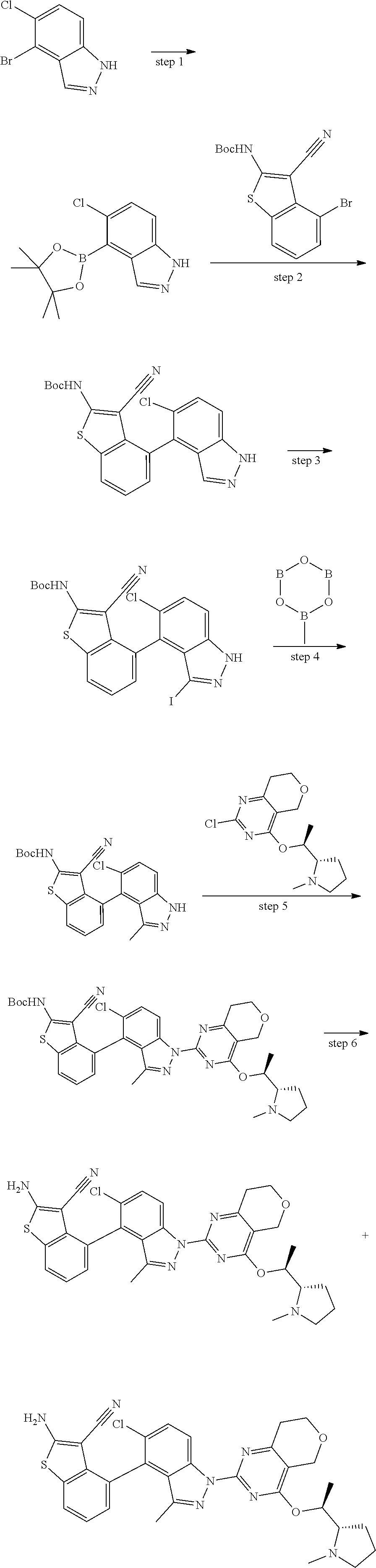

Step 1: Synthesis of 5-chloro-4-(4,4,5,5-tetramethyl-1,3,2-dioxaborolan-2-yl)-1H-indazole

The title compound was obtained as a yellow solid, 800 mg, 70%, from 4-bromo-5-chloro-1H-indazole and 5,5,5′,5′-tetramethyl-2,2′-bi (1,3,2-dioxaborolan) following a similar procedure to that described in Example 23 and 24, step 1. LCMS: m/z=279 [M+H]⁺.

Step 2: Synthesis of tert-butyl (4-(5-chloro-1H-indazol-4-yl)-3-cyanobenzo[b]thiophen-2-yl)carbamate

To a stirred solution of the title compound from step 1 (788.2 mg, 2.83 mmol) in dioxane/H₂O (15 mL/3 mL) was added Pd(dtbpf)Cl₂ (184 mg, 283 mmol) and tert-butyl (4-bromo-3-cyanobenzo[b]thiophen-2-yl)carbamate and the reaction mixture was stirred for 2 h at 80° C. Water (20 mL) was added to the cooled reaction and the mixture was extracted with DCM (3×30 mL). The combined organic layers were dried over Na₂SO₄ and concentrated. The crude product was purified by silica gel column with PE:EtOAc=2:1 to give the title compound (800 mg, 67%) as a yellow solid. LCMS: m/z=425 [M+H]⁺.

Step 3: Synthesis of tert-butyl (4-(5-chloro-3-iodo-1H-indazol-4-yl)-3-cyanobenzo[b]thiophen-2-yl)carbamate

To a stirred solution of the title compound from step 2 (800 mg, 1.64 mmol) in THF (10 mL) was added NIS (553 mg, 2.46 mmol) and the reaction mixture was stirred overnight at rt. Water (20 mL) was added and the resulting solution was extracted with DCM (3×20 mL). The combined organic layers were dried over Na₂SO₄ and concentrated. The crude product was purified by silica gel column with PE:EtOAc=1:1 to give the title compound (490 mg, 54%) as a brown solid. LCMS: m/z=549 [M−1]⁻.

Step 4: Synthesis of tert-butyl (4-(5-chloro-3-methyl-1H-indazol-4-yl)-3-cyanobenzo[b]thiophen-2-yl)carbamate

To a stirred solution of the title compound from step 3 (450 mg, 816 μmol) in dioxane/H₂O (6 mL/1.5 mL) were added 4,6-trimethyl-1,3,5,2,4,6-trioxatriborinane (306 mg, 2.44 mmol), K₂CO₃ (225 mg, 1.63 mmol) and Pd(dppf)Cl₂ (59.2 mg, 81.6 μmol) under N₂ and the reaction mixture was stirred for 2 h at 100° C. The cooled mixture was diluted with water (20 mL) and the resulting solution was extracted with DCM (3×20 mL). The combined organic layers were dried over Na₂SO₄ and concentrated. The crude product was purified by silica gel column with PE:EtOAc=1:1 to give the title compound (120 mg, 33%) as a yellow solid. LCMS: m/z=439 [M+H]⁺.

Step 5: Synthesis of tert-butyl (4-(5-chloro-3-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-3-cyanobenzo[b]thiophen-2-yl)carbamate

The title compound was obtained as a brown solid, 20 mg, 13%, from the title compound from step 4 and Intermediate 1, following a similar procedure to that described in Example 77 and 78, step 2. LCMS: m/z=700 [M+H]⁺.

Step 6: Synthesis of (S)-2-amino-4-(5-chloro-3-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophene-3-carbonitrile and (R)-2-amino-4-(5-chloro-3-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophene-3-carbonitrile

The title compounds were obtained from the title compound from step 5, following a similar procedure to that described in Example 77 and 78, step 3.

Peak 1, Example 83, (1.6 mg, 16%) as a white solid, LCMS: m/z=600 [M+H]+. 1H NMR (400 MHz, DMSO-d₆) δ 8.68 (d, 1H), 7.82 (d, 1H), 7.77 (s, 2H), 7.69 (d, 1H), 7.26 (t, 1H), 7.18 (d, 1H), 5.46 (t, 1H), 4.66-4.52 (m, 2H), 4.00 (d, 2H), 3.04-2.96 (m, 1H), 2.90 (t, 2H), 2.80-2.67 (m, 1H), 2.46 (s, 3H), 2.32 (d, 1H), 1.86 (d, 1H), 1.70 (s, 3H), 1.36 (d, 3H), 1.30 (s, 3H).

Peak 2, Example 84, (1.5 mg, 15%) as a white solid. LCMS: m/z=600 [M+H]⁺ 1H NMR (400 MHz, DMSO-d₆) δ 8.67 (d, 1H), 7.82 (d, 1H), 7.75 (s, 2H), 7.69 (d, 1H), 7.26 (t, 1H), 7.18 (d, 1H), 5.44 (p, 1H), 4.65-4.51 (m, 2H), 4.00 (t, 2H), 2.97 (d, 1H), 2.90 (t, 2H), 2.80-2.67 (m, 1H), 2.39 (s, 3H), 2.20 (t, 1H), 1.89-1.78 (m, 1H), 1.75-1.70 (m, 6H), 1.33 (d, 3H).

### Example 85

### (S)-2-amino-4-(5-chloro-3,7-difluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (R)-2-amino-4-(5-chloro-3,7-difluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

### And Example 86

### (R)-2-amino-4-(5-chloro-3,7-difluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (S)-2-amino-4-(5-chloro-3,7-difluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

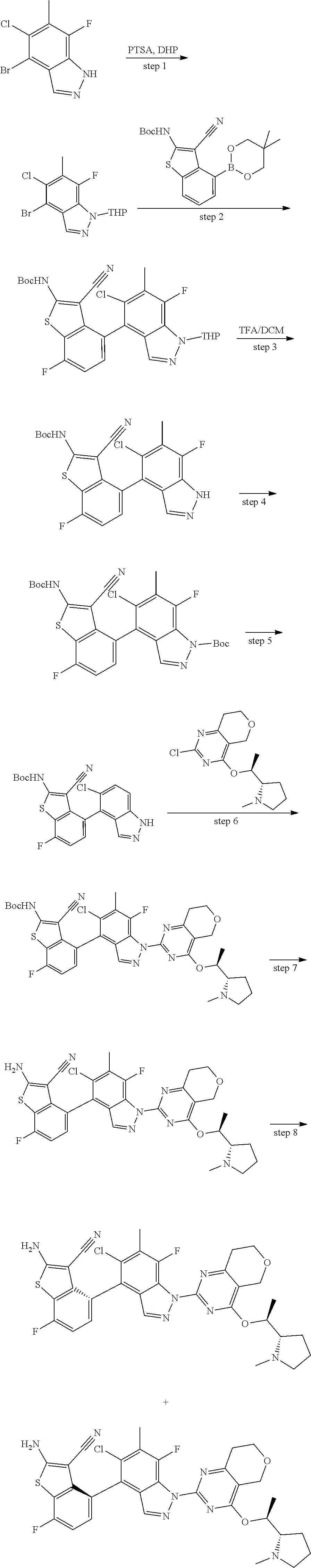

Step 1: Synthesis of 4-bromo-5-chloro-3,7-difluoro-6-methyl-1-(tetrahydro-2H-pyran-2-yl)-1H-indazole

To a stirred solution of 4-bromo-5-chloro-3,7-difluoro-6-methyl-1H-indazole (Example 27 and 28, step 1, 1.2 g, 4.26 mmol) in DCM (20 mL) were added DHP (1.07 g, 12.8 mmol) and TsOH (81 mg, 426 μmol) and the reaction mixture was stirred for 2 h at rt and concentrated. The crude product was purified by silica gel column with PE:EtOAc=20:1 to give the title compound (1.4 g, 89%) as a white solid. 1H NMR (400 MHz, DMSO-d₆) δ 5.77 (m, 1H), 3.88-3.45 (m, 2H), 2.44 (s, 3H), 2.39-1.86 (m, 4H), 1.63 (d, 2H).

Step 2: Synthesis of tert-butyl (4-(5-chloro-3,7-difluoro-6-methyl-1-(tetrahydro-2H-pyran-2-yl)-1H-indazol-4-yl)-3-cyano-7-fluorobenzo[b]thiophen-2-yl)carbamate

The title compound was obtained as a brown solid, 400 mg, 19%, from the title compound from step 1 and Intermediate 9, following a similar procedure to that described in Example 83 and 84, step 1 step 2. LCMS: m/z=577 [M+H]⁺.

Step 3: Synthesis of 2-amino-4-(5-chloro-3,7-difluoro-6-methyl-1H-indazol-4-yl)-7-fluorobenzo[b]thiophene-3-carbonitrile

The title compound was obtained as a brown solid, 190 mg, 70%, from the title compound from step 2, following a similar procedure to that described in Example 1 and 2, step 2. LCMS m/z=393 [M+H]+

Step 4: Synthesis of tert-butyl 4-(2-((tert-butoxycarbonyl)amino)-3-cyano-7-fluorobenzo[b]thiophen-4-yl)-5-chloro-3,7-difluoro-6-methyl-1H-indazole-1-carboxylate

The title compound was obtained as a yellow solid, 190 mg, 70%, from the title compound from step 3, following a similar procedure to that described in Example 27 and 28, step 5. LCMS m/z=591 [M−H]−

Step 5: Synthesis of tert-butyl (4-(5-chloro-3,7-difluoro-6-methyl-1H-indazol-4-yl)-3-cyano-7-fluorobenzo[b]thiophen-2-yl)carbamate

The title compound was obtained as a yellow solid, 120 mg, 76%, from the title compound from step 4, following a similar procedure to that described in Example 27 and 28, step 6. LCMS: m/z=491 [M−H]⁻.

Step 6: Synthesis of tert-butyl (4-(5-chloro-3,7-difluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-3-cyano-7-fluorobenzo[b]thiophen-2-yl)carbamate

The title compound was obtained as a brown solid, 75 mg, 45%, from the title compound from step 5 and Intermediate 1, following a similar procedure to that described in Example 77 and 78, step 2. LCMS: m/z=754 [M+H]⁺.

Step 7: Synthesis(S)-2-amino-4-(5-chloro-3,7-difluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile and (R)-2-amino-4-(5-chloro-3,7-difluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

The title compounds were obtained from the title compound from step 6, following a similar procedure to that described in Example 77 and 78, step 3.

Peak 1, Example 85, (6.1 mg, 14%) as a white solid. LCMS: m/z=654 [M+H]⁺; 1H NMR (400 MHz, DMSO-d₆) δ 8.10 (s, 2H), 7.33 (dd, 1H), 7.16 (dd, 1H), 5.41 (p, 1H), 4.68-4.54 (m, 2H), 4.00 (t, 2H), 2.97 (ddd, 1H), 2.87 (t, 2H), 2.64 (d, 1H), 2.48 (d, 3H), 2.33 (s, 3H), 2.21 (td, 1H), 1.82-1.65 (m, 4H), 1.26 (d, 3H).

Peak 2, Example 86, (10.9 mg, 25%) as a white solid. LCMS: m/z=654 [M+H]⁺, 1H NMR (400 MHz, DMSO-d₆) δ 8.09 (s, 2H), 7.33 (dd, 1H), 7.16 (dd, 1H), 5.35 (p, 1H), 4.68-4.54 (m, 2H), 4.00 (t, 2H), 2.97-2.95 (m, 1H), 2.87-2.81 (m, 2H), 2.64 (d, 1H), 2.47 (d, 3H), 2.36-2.30 (m, 4H), 1.83-1.65 (m, 4H), 1.26 (d, 3H).

### Example 87

### (S)-2-amino-4-(5-chloro-7-fluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (R)-2-amino-4-(5-chloro-7-fluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

### And Example 88

### (R)-2-amino-4-(5-chloro-7-fluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (S)-2-amino-4-(5-chloro-7-fluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

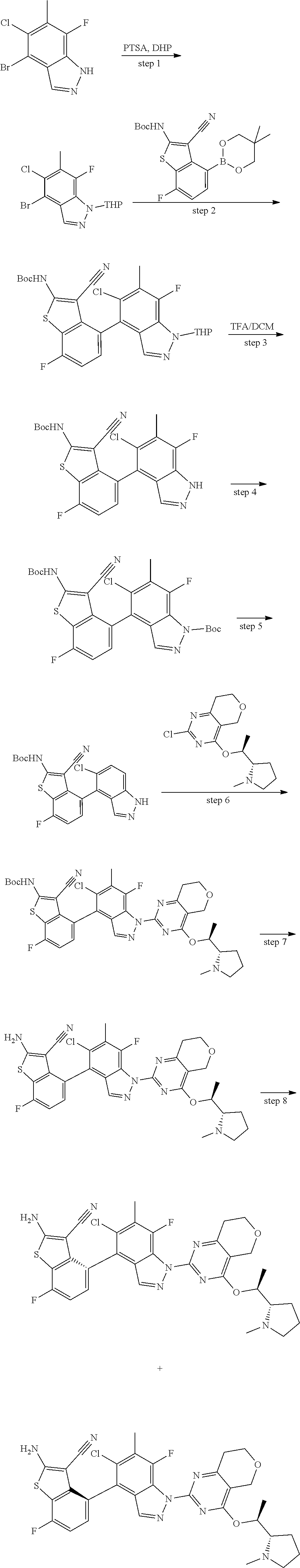

Step 1: Synthesis of 4-bromo-5-chloro-7-fluoro-6-methyl-1-(tetrahydro-2H-pyran-2-yl)-1H-indazole4-bromo-5-chloro-7-fluoro-6-methyl-1-(tetrahydro-2H-pyran-2-yl)-1H-indazole

The title compound was obtained as a white solid, 1 g, 75%, from 4-bromo-5-chloro-7-fluoro-6-methyl-1H-indazole (Example 27 and 28, step 1), following the procedure described in Example 85 and 86, step 1. 1H NMR (400 MHz, DMSO-d₆) δ 8.15 (d, 1H), 5.81 (td, 1H), 3.93-3.85 (m, 1H), 3.68-3.57 (m, 1H), 2.43-2.31 (m, 4H), 2.12-2.03 (m, 2H), 1.79-1.70 (m, 1H), 1.56 (td, 2H).

Step 2: Synthesis of tert-butyl (4-(5-chloro-7-fluoro-6-methyl-1-(tetrahydro-2H-pyran-2-yl)-1H-indazol-4-yl)-3-cyano-7-fluorobenzo[b]thiophen-2-yl)carbamate

The title compound was obtained as a brown solid, 500 mg, 30%, from the title compound of step 1 and Intermediate 9 following the procedure described in Example 85 and 86, step 2. LCMS m/z=559 [M+H]+

Step 3: Synthesis of 2-amino-4-(5-chloro-7-fluoro-6-methyl-1H-indazol-4-yl)-7-fluorobenzo[b]thiophene-3-carbonitrile

The title compound was obtained as a brown solid, 220 mg, 71%, from the title compound of step 2, following the procedure described in Example 85 and 86, step 3. LCMS m/z=375 [M+H]+

Step 4: Synthesis of tert-butyl 4-(2-((tert-butoxycarbonyl)amino)-3-cyano-7-fluorobenzo[b]thiophen-4-yl)-5-chloro-7-fluoro-6-methyl-1H-indazole-1-carboxylate

The title compound was obtained as a brown solid, 230 mg, 71%, from the title compound of step 3, following the procedure described in Example 27 and 28, step 5. LCMS m/z=573 [M−H]−

Step 5: Synthesis of tert-butyl (4-(5-chloro-7-fluoro-6-methyl-1H-indazol-4-yl)-3-cyano-7-fluorobenzo[b]thiophen-2-yl)carbamate

The title compound was obtained as a yellow solid, 140 mg, 74%, from the title compound of step 4, following the procedure described in Example 27 and 28, step 6. LCMS m/z=473 [M−H]−

Step 6: Synthesis of tert-butyl (4-(5-chloro-7-fluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-3-cyano-7-fluorobenzo[b]thiophen-2-yl)carbamate

The title compound was obtained as a brown solid, 120 mg, 51%, from the title compound of step 5 and Intermediate 1, following a similar procedure to that described in Example 77 and 78, step 2. LCMS m/z=736 [M+H]+

Step 7: Synthesis of (S)-2-amino-4-(5-chloro-7-fluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile and (R)-2-amino-4-(5-chloro-7-fluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol -4-yl)-7-fluorobenzo[b]thiophene-3-carbonitrile

2-Amino-4-(5-chloro-7-fluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile was obtained from the title compound from step 6, following a similar procedure to that described in Example 1 and 2, step 2. This solid was purified by Prep-Chiral-SFC (Column: CHIRALPAK ID, 2*25 cm, 5 μm; Mobile Phase A: Hex (0.2% IPA), Mobile Phase B: EtOH: DCM=1:1; Flow rate: 20 mL/min; Gradient: 15% B isocratic for 30 min, to give Peak 1, Example 87 (13 mg, 20%) as a white solid and Peak 2, Example 88 (4.7 mg, 6%) as a white solid.

Example 87 LCMS: m/z=636 [M+H]⁺; 1H NMR (400 MHz, DMSO-d₆) δ 8.04 (s, 2H), 7.92 (d, 1H), 7.28 (dd, 1H), 7.17 (t, 1H), 5.42 (p, 1H), 4.70-4.56 (m, 2H), 4.01 (t, 2H), 3.00 (d, 1H), 2.88 (t, 2H), 2.73-2.67 (m, 1H), 2.46 (s, 3H), 2.37-2.29 (m, 4H), 1.85-1.69 (m, 4H), 1.28 (d, 3H).

Example 88 LCMS: m/z=636 [M+H]⁺; 1H NMR (400 MHz, DMSO-d₆) δ 8.09 (s, 2H), 7.91 (d, 1H), 7.33 (dd, 1H), 7.16 (dd, 1H), 5.35 (p, 1H), 4.68-4.54 (m, 2H), 4.00 (t, 2H), 2.97 (d, 1H), 2.87 (t, 2H), 2.67-2.60 (m, 1H), 2.47 (s, 3H), 2.36-2.30 (m, 4H), 1.83-1.63 (s, 4H), 1.27 (d, 3H).

### Example 89

### (R)-2-amino-4-(5-chloro-3-methyl-1-(4-((S)-1-((S)-1-methylpyrrolidin-2-yl)ethoxy)-6,7-dihydro-5H-pyrano[2,3-d]pyrimidin-2-yl)-1H-indazol-4-yl)-7-fluorobenzo[b]thiophene-3-carbonitrile or (S)-2-amino-4-(5-chloro-3-methyl-1-(4-((S)-1-((S)-1-methylpyrrolidin-2-yl)ethoxy)-6,7-dihydro-5H-pyrano[2,3-d]pyrimidin-2-yl)-1H-indazol-4-yl)-7-fluorobenzo[b]thiophene-3-carbonitrile

### And Example 90

### (S)-2-amino-4-(5-chloro-3-methyl-1-(4-((S)-1-((S)-1-methylpyrrolidin-2-yl)ethoxy)-6,7-dihydro-5H-pyrano[2,3-d]pyrimidin-2-yl)-1H-indazol-4-yl)-7-fluorobenzo[b]thiophene-3-carbonitrile or (R)-2-amino-4-(5-chloro-3-methyl-1-(4-((S)-1-((S)-1-methylpyrrolidin-2-yl)ethoxy)-6,7-dihydro-5H-pyrano[2,3-d]pyrimidin-2-yl)-1H-indazol -4-yl)-7-fluorobenzo[b]thiophene-3-carbonitrile

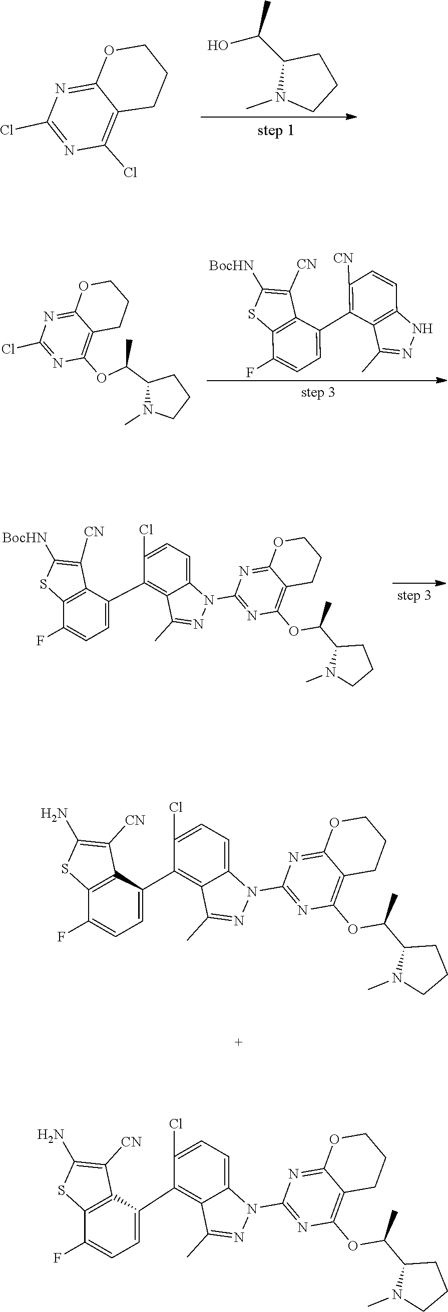

Step 1: Synthesis of 2-chloro-4-((S)-1-((S)-1-methylpyrrolidin-2-yl)ethoxy)-6,7-dihydro-5H-pyrano[2,3-d]pyrimidine

To a stirred solution of (S)-1-((S)-1-methylpyrrolidin-2-yl)ethan-1-ol (188 mg, 1.46 mmol) in THF (3.0 mL) was added NaH (87.4 mg, 2.19 mmol) at 0° C. and the mixture was stirred for 10 mins at rt. 2,4-Dichloro-6,7-dihydro-5H-pyrano[2,3-d]pyrimidine (300 mg, 1.46 mmol) was added and the reaction was stirred for 2 h at 60° C. The mixture was cooled to rt and water added. The resulting solution was extracted with DCM (3×30 mL) and the combined organic layers were dried over Na₂SO₄ and concentrated. The crude product was purified by silica gel column with DCM:MeOH=10:1 to give the title compound (140 mg, 31%) as a yellow solid. LCMS: m/z=298 [M+H]+.

Step 2: Synthesis of tert-butyl (4-(5-chloro-3-methyl-1-(4-((S)-1-((S)-1-methylpyrrolidin-2-yl)ethoxy)-6,7-dihydro-5H-pyrano[2,3-d]pyrimidin-2-yl)-1H-indazol-4-yl)-3-cyano-7-fluorobenzo[b]thiophen-2-yl)carbamate

The title compound was obtained as a yellow solid, 210 mg, 73%, from the title compound from step 1 and Intermediate 15, following a similar procedure to that described in Example 77 and 78, step 2. LCMS m/z=718 [M+H]+

Step 3: Synthesis of (R)-2-amino-4-(5-chloro-3-methyl-1-(4-((S)-1-((S)-1-methylpyrrolidin-2-yl)ethoxy)-6,7-dihydro-5H-pyrano[2,3-d]pyrimidin-2-yl)-1H-indazol-4-yl)-7-fluorobenzo[b]thiophene-3-carbonitrile and (S)-2-amino-4-(5-chloro-3-methyl-1-(4-((S)-1-((S)-1-methylpyrrolidin-2-yl)ethoxy)-6,7-dihydro-5H-pyrano[2,3-d]pyrimidin-2-yl)-1H-indazol-4-yl)-7-fluorobenzo[b]thiophene-3-carbonitrile

The title compounds were obtained from the title compound from step 2, following a similar procedure to that described in Example 77 and 78, step 3.

Example 89 (47.4 mg, 24%) as a white solid LCMS: m/z=618 [M+H]⁺ 1H NMR (400 MHz, DMSO-d₆) δ 8.64 (d, 1H), 8.04 (s, 2H), 7.70 (d, 1H), 7.25 (dd, 1H), 7.16 (dd, 1H), 5.39 (p, 1H), 4.42-4.34 (m, 2H), 3.04-2.96 (m, 1H), 2.73-2.66 (m, 1H), 2.60-2.55 (m, 2H), 2.43 (s, 3H), 2.30-2.21 (m, 1H), 2.02-1.93 (m, 2H), 1.91-1.80 (m, 1H), 1.79-1.65 (m, 6H), 1.33 (d, 3H).

Example 90 (73.2 mg, 38%) as a white solid LCMS: m/z=618 [M+H]⁺, 1H NMR (400 MHz, DMSO-d₆) δ 8.65 (d, 1H), 8.05 (s, 2H), 7.70 (d, 1H), 7.25 (dd, 1H), 7.16 (t, 1H), 5.42 (q, 1H), 4.38 (t, 2H), 3.06-2.97 (m, 1H), 2.78-2.68 (m, 1H), 2.61-2.51 (m, 2H), 2.44 (s, 3H), 2.28 (q, 1H), 2.02-1.90 (m, 2H), 1.91-1.80 (m, 1H), 1.79-1.62 (m, 6H), 1.34 (d, 3H).

### Example 91

### [(R)-2-amino-4-(5-chloro-1-((S)-6-(methoxymethyl)-4-((S)-1-((S)-1-methylpyrrolidin-2-yl)ethoxy)-6,7-dihydro-5H-cyclopenta[d]pyrimidin-2-yl)-1H-indazol-4-yl)-7-fluorobenzo[b]thiophene-3-carbonitrile or (S)-2-amino-4-(5-chloro-1-((S)-6-(methoxymethyl)-4-((S)-1-((S)-1-methylpyrrolidin-2-yl)ethoxy)-6,7-dihydro-5H-cyclopenta[d]pyrimidin-2-yl)-1H-indazol-4-yl)-7-fluorobenzo[b]thiophene-3-carbonitrile] or [(R)-2-amino-4-(5-chloro-1-((R)-6-(methoxymethyl)-4-((S)-1-((S)-1-methylpyrrolidin-2-yl)ethoxy)-6,7-dihydro-5H-cyclopenta[d]pyrimidin-2-yl)-1H-indazol-4-yl)-7-fluorobenzo[b]thiophene-3-carbonitrile or (S)-2-amino-4-(5-chloro-1-((R)-6-(methoxymethyl)-4-((S)-1-((S)-1-methylpyrrolidin-2-yl)ethoxy)-6,7-dihydro-5H-cyclopenta[d]pyrimidin-2-yl)-1H-indazol-4-yl)-7-fluorobenzo[b]thiophene-3-carbonitrile]

### And Example 92

### [(S)-2-amino-4-(5-chloro-1-((S)-6-(methoxymethyl)-4-((S)-1-((S)-1-methylpyrrolidin-2-yl)ethoxy)-6,7-dihydro-5H-cyclopenta[d]pyrimidin-2-yl)-1H-indazol-4-yl)-7-fluorobenzo[b]thiophene-3-carbonitrile or (R)-2-amino-4-(5-chloro-1-((S)-6-(methoxymethyl)-4-((S)-1-((S)-1-methylpyrrolidin-2-yl)ethoxy)-6,7-dihydro-5H-cyclopenta[d]pyrimidin-2-yl)-1H-indazol-4-yl)-7-fluorobenzo[b]thiophene-3-carbonitrile] or [(S)-2-amino-4-(5-chloro-1-((R)-6-(methoxymethyl)-4-((S)-1-((S)-1-methylpyrrolidin-2-yl)ethoxy)-6,7-dihydro-5H-cyclopenta[d]pyrimidin-2-yl)-1H-indazol-4-yl)-7-fluorobenzo[b]thiophene-3-carbonitrile or (R)-2-amino-4-(5-chloro-1-((R)-6-(methoxymethyl)-4-((S)-1-((S)-1-methylpyrrolidin-2-yl)ethoxy)-6,7-dihydro-5H-cyclopenta[d]pyrimidin-2-yl)-1H-indazol-4-yl)-7-fluorobenzo[b]thiophene-3-carbonitrile]

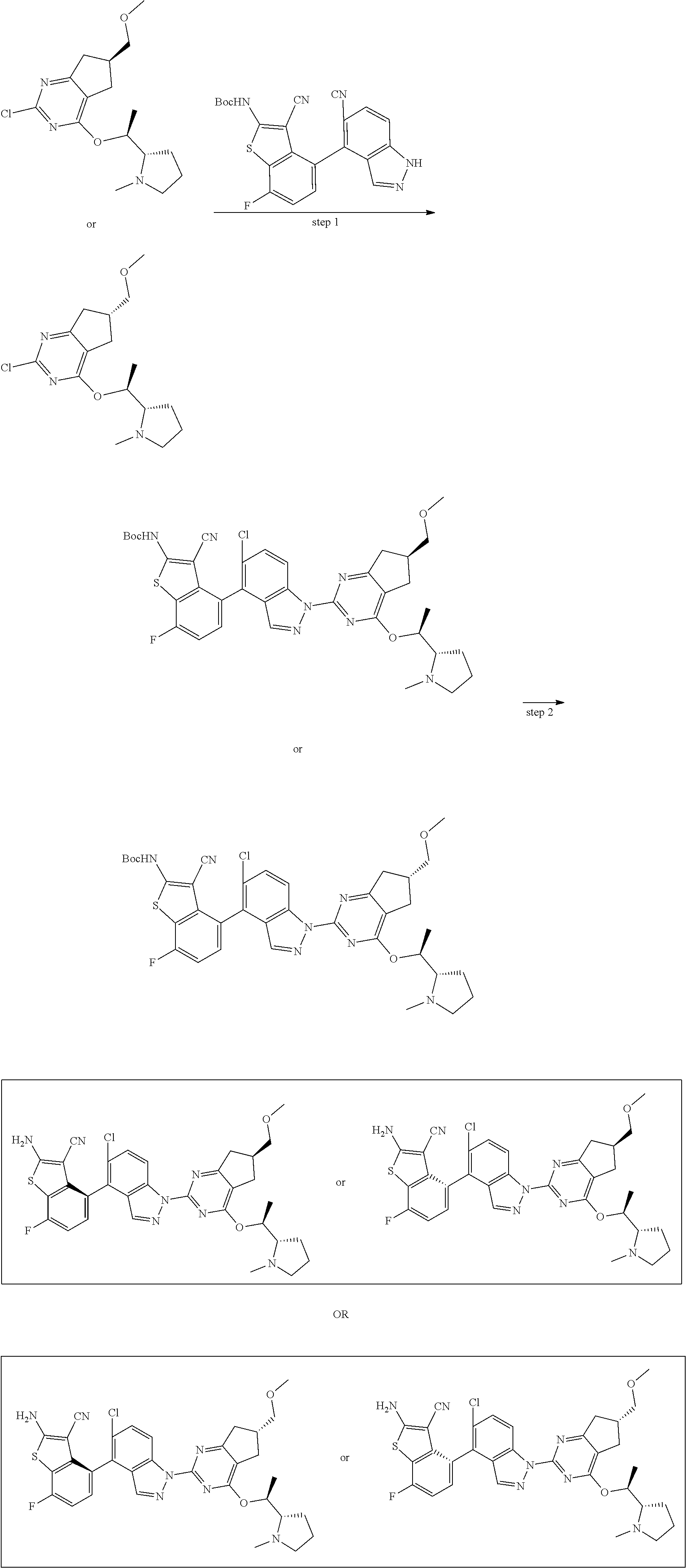

Step 1: Synthesis of tert-butyl (4-(5-chloro-1-((S)-6-(methoxymethyl)-4-((S)-1-((S)-1-methylpyrrolidin-2-yl)ethoxy)-6,7-dihydro-5H-cyclopenta[d]pyrimidin-2-yl)-1H-indazol-4-yl)-3-cyano-7-fluorobenzo[b]thiophen-2-yl)carbamate or tert-butyl (4-(5-chloro-1-((R)-6-(methoxymethyl)-4-((S)-1-((S)-1-methylpyrrolidin-2-yl)ethoxy)-6,7-dihydro-5H-cyclopenta[d]pyrimidin-2-yl)-1H-indazol-4-yl)-3-cyano-7-fluorobenzo[b]thiophen-2-yl)carbamate

The title compound was obtained as a yellow solid, 150 mg, 67%, from Intermediate 6A and Intermediate 16, following a similar procedure to that described in Example 77 and 78, step 2. LCMS: m/z=732 [M+H]+.

Step 2: Synthesis of [(R)-2-amino-4-(5-chloro-1-((S)-6-(methoxymethyl)-4-((S)-1-((S)-1-methylpyrrolidin-2-yl)ethoxy)-6,7-dihydro-5H-cyclopenta[d]pyrimidin-2-yl)-1H-indazol-4-yl)-7-fluorobenzo[b]thiophene-3-carbonitrile and (S)-2-amino-4-(5-chloro-1-((S)-6-(methoxymethyl)-4-((S)-1-((S)-1-methylpyrrolidin-2-yl)ethoxy)-6,7-dihydro-5H-cyclopenta[d]pyrimidin-2-yl)-1H-indazol-4-yl)-7-fluorobenzo[b]thiophene-3-carbonitrile] or [(R)-2-amino-4-(5-chloro-1-((R)-6-(methoxymethyl)-4-((S)-1-((S)-1-methylpyrrolidin-2-yl)ethoxy)-6,7-dihydro-5H-cyclopenta[d]pyrimidin-2-yl)-1H-indazol-4-yl)-7-fluorobenzo[b]thiophene-3-carbonitrile and (S)-2-amino-4-(5-chloro-1-((R)-6-(methoxymethyl)-4-((S)-1-((S)-1-methylpyrrolidin-2-yl)ethoxy)-6,7-dihydro-5H-cyclopenta[d]pyrimidin-2-yl)-1H-indazol-4-yl)-7-fluorobenzo[b]thiophene-3-carbonitrile]

The title compounds were obtained from the title compound of step 1, following a similar procedure to that described in Example 77 and 78, step 3.

Example 91, 53.0 mg, 41%, as a white solid. LCMS: m/z=632 [M+H]⁺ 1H NMR (400 MHz, DMSO-d₆) δ 8.66 (d, 1H), 8.03 (s, 2H), 7.90 (s, 1H), 7.72 (d, 1H), 7.28 (dd, 1H), 7.17 (dd, 1H), 5.40 (p, 1H), 3.39 (d, 2H), 3.29 (s, 3H), 3.18-3.06 (m, 1H), 3.01-2.88 (m, 2H), 2.87-2.74 (m, 2H), 2.68-2.55 (m, 2H), 2.40 (s, 3H), 2.23 (q, 1H), 1.94-1.81 (m, 1H), 1.80-1.62 (m, 3H), 1.35 (d, 3H).

Example 92, 56.2 mg 44% as a white solid. LCMS: m/z=632 [M+H]⁺; 1H NMR (400 MHz, DMSO-d₆) δ 8.66 (d, 1H), 8.03 (s, 2H), 7.90 (s, 1H), 7.72 (d, 1H), 7.28 (dd, 1H), 7.17 (t, 1H), 5.41 (p, 1H), 3.40 (d, 2H), 3.29 (s, 3H), 3.18-3.07 (m, 1H), 3.02-2.74 (m, 4H), 2.70-2.55 (m, 2H), 2.41 (s, 3H), 2.30-2.19 (m, 1H), 1.93-1.79 (m, 1H), 1.78-1.61 (m, 3H), 1.36 (d, 3H).

### Example 93

### [(R)-2-amino-4-(5-chloro-1-((R)-6-(methoxymethyl)-4-((S)-1-((S)-1-methylpyrrolidin-2-yl)ethoxy)-6,7-dihydro-5H-cyclopenta[d]pyrimidin-2-yl)-1H-indazol-4-yl)-7-fluorobenzo[b]thiophene-3-carbonitrile or (S)-2-amino-4-(5-chloro-1-((R)-6-(methoxymethyl)-4-((S)-1-((S)-1-methylpyrrolidin-2-yl)ethoxy)-6,7-dihydro-5H-cyclopenta[d]pyrimidin-2-yl)-1H-indazol-4-yl)-7-fluorobenzo[b]thiophene-3-carbonitrile] or [(R)-2-amino-4-(5-chloro-1-((S)-6-(methoxymethyl)-4-((S)-1-((S)-1-methylpyrrolidin-2-yl)ethoxy)-6,7-dihydro-5H-cyclopenta[d]pyrimidin-2-yl)-1H-indazol-4-yl)-7-fluorobenzo[b]thiophene-3-carbonitrile or (S)-2-amino-4-(5-chloro-1-((S)-6-(methoxymethyl)-4-((S)-1-((S)-1-methylpyrrolidin-2-yl)ethoxy)-6,7-dihydro-5H-cyclopenta[d]pyrimidin-2-yl)-1H-indazol-4-yl)-7-fluorobenzo[b]thiophene-3-carbonitrile]

### And Example 94

### [(S)-2-amino-4-(5-chloro-1-((R)-6-(methoxymethyl)-4-((S)-1-((S)-1-methylpyrrolidin-2-yl)ethoxy)-6,7-dihydro-5H-cyclopenta[d]pyrimidin-2-yl)-1H-indazol-4-yl)-7-fluorobenzo[b]thiophene-3-carbonitrile or (R)-2-amino-4-(5-chloro-1-((R)-6-(methoxymethyl)-4-((S)-1-((S)-1-methylpyrrolidin-2-yl)ethoxy)-6,7-dihydro-5H-cyclopenta[d]pyrimidin-2-yl)-1H-indazol-4-yl)-7-fluorobenzo[b]thiophene-3-carbonitrile] or [(S)-2-amino-4-(5-chloro-1-((S)-6-(methoxymethyl)-4-((S)-1-((S)-1-methylpyrrolidin-2-yl)ethoxy)-6,7-dihydro-5H-cyclopenta[d]pyrimidin-2-yl)-1H-indazol-4-yl)-7-fluorobenzo[b]thiophene-3-carbonitrile or (R)-2-amino-4-(5-chloro-1-((S)-6-(methoxymethyl)-4-((S)-1-((S)-1-methylpyrrolidin-2-yl)ethoxy)-6,7-dihydro-5H-cyclopenta[d]pyrimidin-2-yl)-1H-indazol-4-yl)-7-fluorobenzo[b]thiophene-3-carbonitrile]

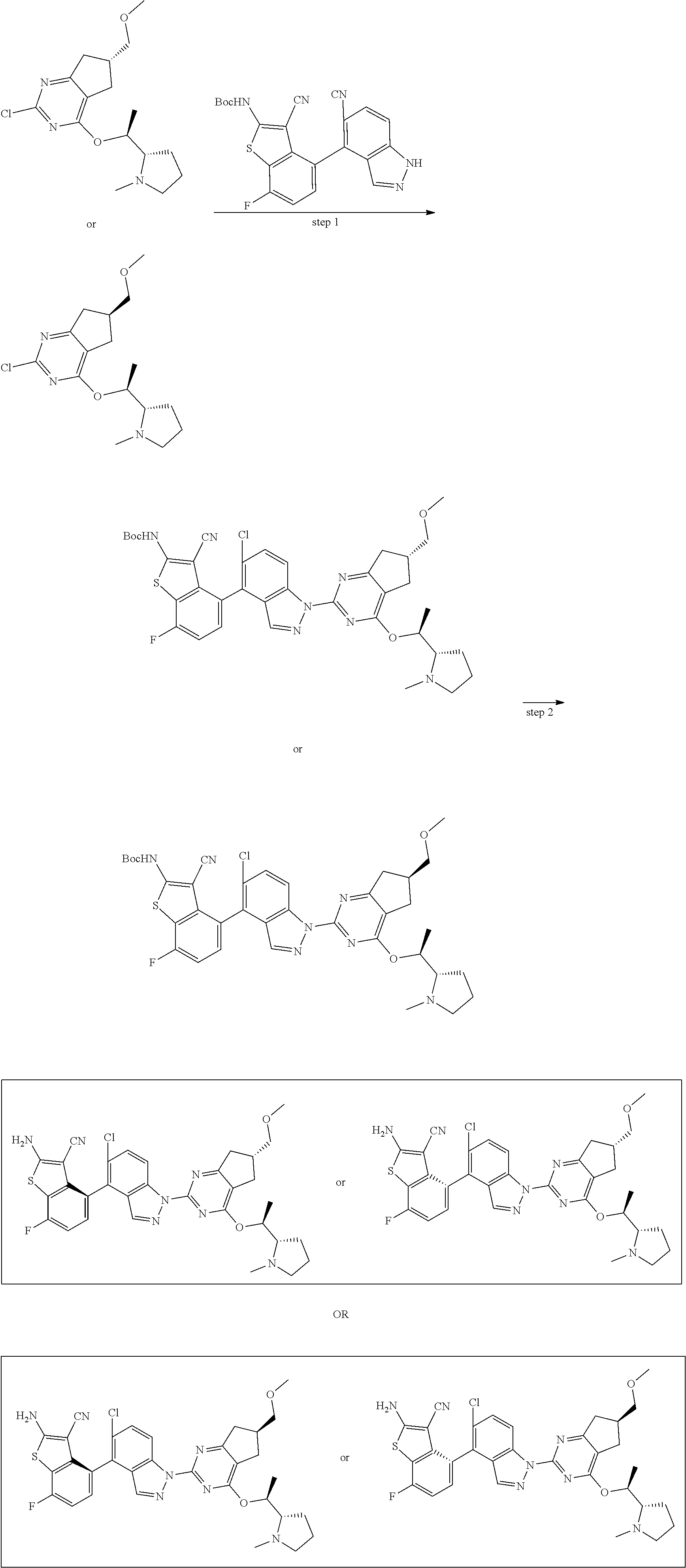

Step 1: Synthesis of tert-butyl (4-(5-chloro-1-((R)-6-(methoxymethyl)-4-((S)-1-((S)-1-methylpyrrolidin-2-yl)ethoxy)-6,7-dihydro-5H-cyclopenta[d]pyrimidin-2-yl)-1H-indazol-4-yl)-3-cyano-7-fluorobenzo[b]thiophen-2-yl)carbamate or tert-butyl (4-(5-chloro-1-((S)-6-(methoxymethyl)-4-((S)-1-((S)-1-methylpyrrolidin-2-yl)ethoxy)-6,7-dihydro-5H-cyclopenta[d]pyrimidin-2-yl)-1H-indazol-4-yl)-3-cyano-7-fluorobenzo[b]thiophen-2-yl)carbamate

The title compound was obtained as a yellow solid, 140 mg, 63%, from Intermediate 6B and Intermediate 16, following the procedure described in Example 77 and 78, step 2. LCMS m/z=732 [M+H]+

Step 2: Synthesis [(R)-2-amino-4-(5-chloro-1-((R)-6-(methoxymethyl)-4-((S)-1-((S)-1-methylpyrrolidin-2-yl)ethoxy)-6,7-dihydro-5H-cyclopenta[d]pyrimidin-2-yl)-1H-indazol-4-yl)-7-fluorobenzo[b]thiophene-3-carbonitrile and (S)-2-amino-4-(5-chloro-1-((R)-6-(methoxymethyl)-4-((S)-1-((S)-1-methylpyrrolidin-2-yl)ethoxy)-6,7-dihydro-5H-cyclopenta[d]pyrimidin-2-yl)-1H-indazol-4-yl)-7-fluorobenzo[b]thiophene-3-carbonitrile] or [(R)-2-amino-4-(5-chloro-1-((S)-6-(methoxymethyl)-4-((S)-1-((S)-1-methylpyrrolidin-2-yl)ethoxy)-6,7-dihydro-5H-cyclopenta[d]pyrimidin-2-yl)-1H-indazol-4-yl)-7-fluorobenzo[b]thiophene-3-carbonitrile and (S)-2-amino-4-(5-chloro-1-((S)-6-(methoxymethyl)-4-((S)-1-((S)-1-methylpyrrolidin-2-yl)ethoxy)-6,7-dihydro-5H-cyclopenta[d]pyrimidin-2-yl)-1H-indazol-4-yl)-7-fluorobenzo[b]thiophene-3-carbonitrile]

The title compounds were obtained from the title compound from step 1, following the procedure described in Example 77 and 78, step 3.

Example 93 (47.6 mg, 40%) as a white solid. LCMS: m/z=632 [M+H]⁺; 1H NMR (400 MHz, DMSO-d₆) δ 8.66 (d, 1H), 8.03 (s, 2H), 7.90 (s, 1H), 7.72 (d, 1H), 7.28 (dd, 1H), 7.17 (dd, 1H), 5.40 (p, 1H), 3.39 (d, 2H), 3.29 (s, 3H), 3.18-3.06 (m, 1H), 3.02-2.89 (m, 2H), 2.87-2.74 (m, 2H), 2.71-2.62 (m, 1H), 2.61-2.51 (m, 1H), 2.40 (s, 3H), 2.25 (h, 1H), 1.95-1.81 (m, 1H), 1.79-1.62 (m, 3H), 1.35 (d, 3H).

Example 94 (49.5 mg, 41%) as a white solid. LCMS: m/z=632 [M+H]⁺, 1H NMR (400 MHz, DMSO-d₆) δ 8.67 (d, 1H), 8.03 (s, 2H), 7.90 (s, 1H), 7.72 (d, 1H), 7.28 (dd, 1H), 7.17 (dd, 1H), 5.41 (p, 1H), 3.39 (d, 2H), 3.28 (s, 3H), 3.13 (q, 1H), 3.01-2.90 (m, 2H), 2.88-2.74 (m, 2H), 2.68-2.60 (m, 1H), 2.59-2.53 (m, 1H), 2.39 (s, 3H), 2.23 (td, 1H), 1.92-1.79 (m, 1H), 1.79-1.61 (m, 3H), 1.35 (d, 3H).

### Example 95

### (S)-2-amino-4-(5-chloro-3-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]selenophene-3-carbonitrile and (R)-2-amino-4-(5-chloro-3-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]selenophene-3-carbonitrile

### And Example 96

### (R)-2-amino-4-(5-chloro-3-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]selenophene-3-carbonitrile and (S)-2-amino-4-(5-chloro-3-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]selenophene-3-carbonitrile

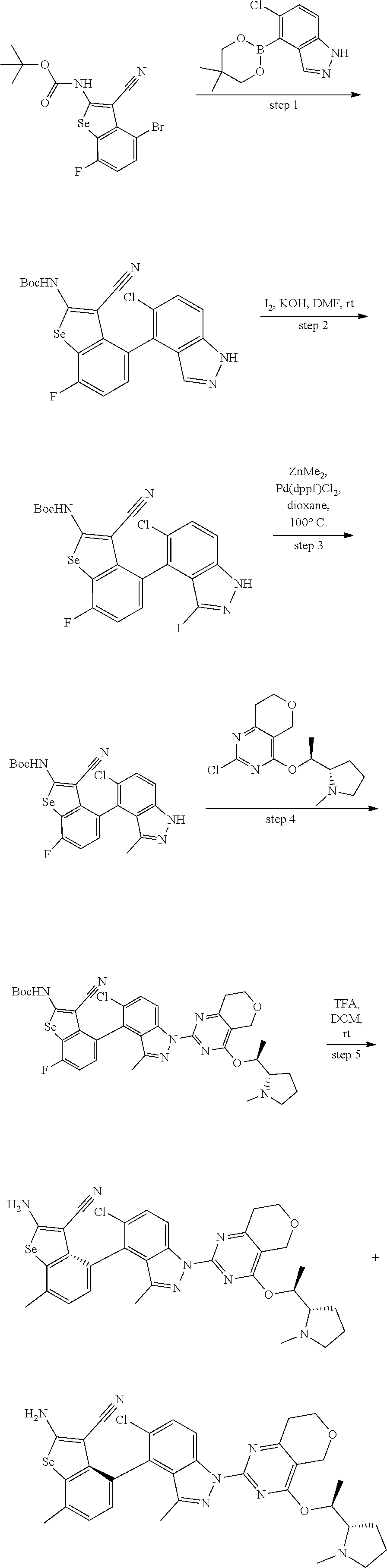

Step 1: Synthesis of tert-butyl (4-(5-chloro-1H-indazol-4-yl)-3-cyano-7-fluorobenzo[b]selenophen-2-yl)carbamate

The title compound was obtained as a yellow solid, 1 g, 85.4%, from Intermediate 20 and 5-chloro-4-(5,5-dimethyl-1,3,2-dioxaborinan-2-yl)-1H-indazole, following a similar procedure to that described in Example 83 and 84, step 2. LCMS m/z=491 [M+H]+

Step 2: Synthesis of tert-butyl (4-(5-chloro-3-iodo-1H-indazol-4-yl)-3-cyano-7-fluorobenzo[b]selenophen-2-yl)carbamate

A mixture of the title compound (900 mg, 1.83 mmol) and KOH (205 mg, 3.66 mmol) in DMF (15 mL) was stirred at 20° C. for 20 min. I₂ (510 mg, 2.01 mmol) in DMF (3 mL) was added, and the reaction mixture was stirred at 20° C. for 6 h. The reaction was poured into water (30 mL) and extracted with EtOAc (20 mL×3). The organic layer was washed with aq. Na₂S₂O₃ (10 mL), dried over Na₂SO₄ and evaporated to afford the title compound (900 mg, crude) as a yellow solid. LCMS: m/z=617 [M+H]⁺

Step 3: Synthesis of tert-butyl (4-(5-chloro-3-methyl-1H-indazol-4-yl)-3-cyano-7-fluorobenzo[b]selenophen-2-yl)carbamate

1M ZnMe₂ (5.64 mL, 5.64 mmol) was added to a mixture of the title compound from step 2 (700 mg, 1.13 mmol) and Pd(dppf)Cl₂·DCM (138 mg, 169 μmol) in dioxane (15 mL) and the reaction mixture was stirred at 100° C. for 30 min under N₂. The reaction was quenched with MeOH (5 mL) and filtered. The filtrate was poured into water (30 mL) and extracted with EtOAc (20 mL×3). The combined organic layer was washed with brine (20 mL), dried over Na₂SO₄ and evaporated. The crude product was purified by Prep-TLC with PE:EtOAc=3:1 to afford the title compound (150 mg, 26.3%) as a yellow solid. LCMS: m/z=505 [M+H]⁺

Step 4: Synthesis of tert-butyl (4-(5-chloro-3-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-3-cyano-7-fluorobenzo[b]selenophen-2-yl)carbamate

The title compound was obtained as a yellow solid, 90 mg, 59.6%, from the title compound from step 3 and Intermediate 1, following a similar procedure to that described in Example 77 and 78, step 2. LCMS: m/z=766 [M+H]⁺

Step 5: Synthesis of (S)-2-amino-4-(5-chloro-3-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]selenophene-3-carbonitrile and (R)-2-amino-4-(5-chloro-3-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]selenophene-3-carbonitrile

The title compounds were obtained from the title compound from step 4, following a similar procedure to that described in Example 66 and 67, step 3.

Peak 1, Example 95 (15.1 mg, 20.4%) as a white solid, LCMS: m/z=666 [M+H]⁺, 1H NMR (400 MHz, DMSO-d₆) δ 8.67 (d, 1H), 8.09 (s, 2H), 7.69 (d, 1H), 7.24 (d, 1H), 7.09 (t, 1H), 5.43 (p, 1H), 4.67-4.54 (m, 2H), 4.01 (t, 2H), 3.20-3.08 (m, 1H), 3.06-2.85 (m, 3H), 2.54 (s, 3H), 2.50-2.39 (m, 1H), 2.07-1.91 (m, 1H), 1.86-1.68 (m, 6H), 1.38 (d, 3H).

Peak 2, Example 96 (23.9 mg, 32.3%) as a white solid. LCMS: m/z=666 [M+H]⁺, 1H NMR (400 MHz, DMSO-d₆) δ 8.67 (d, 1H), 8.09 (s, 2H), 7.68 (d, H), 7.25 (d, 1H), 7.09 (t, 1H), 5.46 (p, 1H), 4.66-4.52 (m, 2H), 4.00 (t, 2H), 3.14-3.01 (m, 1H), 2.94-2.87 (m, 2H), 2.85-2.79 (m, 1H), 2.47 (s, 3H), 2.40-2.31 (m, 1H), 1.97-1.83 (m, 1H), 1.82-1.62 (m, 6H), 1.36 (d, 3H).

### Example 97

### (S)-2-amino-4-(5-chloro-1-(8,8-dimethyl-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (R)-2-amino-4-(5-chloro-1-(8,8-dimethyl-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

### And Example 98

### (R)-2-amino-4-(5-chloro-1-(8,8-dimethyl-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (S)-2-amino-4-(5-chloro-1-(8,8-dimethyl-4-((S)-1-((S)-1-methylpyrrolidin-2-yl)ethoxy)-7,8-dihydro-5H-pyrano[4,3-d]pyrimidin-2-yl)-1H-indazol -4-yl)-7-fluorobenzo[b]thiophene-3-carbonitrile

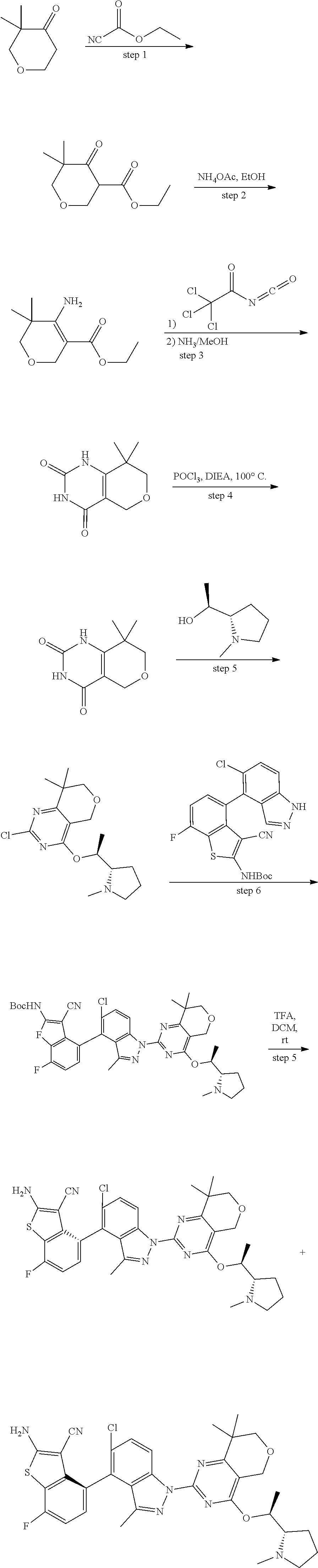

Step 1: Synthesis of ethyl 5,5-dimethyl-4-oxotetrahydro-2H-pyran-3-carboxylate

The title compound was obtained as a yellow solid, 600 mg, 38.4%, from,3-dimethyloxan-4-one and ethyl carbonocyanidate, following a similar procedure to that described in Intermediate 5A and B, step 1. LCMS m/z=199 [M−H]−

Step 2: Synthesis of ethyl 4-amino-5,5-dimethyl-5,6-dihydro-2H-pyran-3-carboxylate

A mixture of the title compound from step 1 (580 mg, 2.89 mmol) and NH₄OAc (222 mg, 2.89 mmol) in EtOH (10 mL) was stirred at 80° C. for 16 h. The reaction mixture was diluted with EtOAc (100 mL), washed with water (100 mL×3) and saturated brine (100 mL). The organic layer was dried over Na₂SO₄, filtered and concentrated in vacuo. The crude product was purified by silica gel column with DCM:MeOH=20:1 to give the title compound (450 mg, 78.2%) as a yellow solid. LCMS m/z=200 [M+H]+

Step 3: Synthesis of 8,8-dimethyl-1,5,7,8-tetrahydro-2H-pyrano[4,3-d]pyrimidine-2,4 (3H)-dione

A mixture of trichloroacetyl isocyanate (423 mg, 2.25 mmol) and the title compound from step 2 (450 mg, 2.25 mmol) in MeCN (5 mL) was stirred at rt for 0.5 h. The reaction mixture was evaporated under reduced pressure, the residue dissolved in 7N NH₃ in MeOH (4 mL) and the solution stirred at 50° C. for 6 h. The reaction mixture was diluted with EtOAc (100 mL), washed with water (100 mL×3) and saturated brine (100 mL). The organic layer was dried over Na₂SO₄, filtered and concentrated in vacuo. The crude product was purified by silica gel column with DCM:MeOH=20:1 to give the title compound (400 mg, 90.7%) as a yellow solid. LCMS m/z=197 [M+H]+

Step 4: Synthesis of 2,4-dichloro-8,8-dimethyl-7,8-dihydro-5H-pyrano[4,3-d]pyrimidine

The title compound was obtained as a yellow solid, 350 mg, 73.9%, from the title compound from step 3, following a similar procedure to that described in Intermediate 5A and B, step 4. LCMS m/z=233 [M+H]+

Step 5: Synthesis of 2-chloro-8,8-dimethyl-4-((S)-1-((S)-1-methylpyrrolidin-2-yl)ethoxy)-7,8-dihydro-5H-pyrano[4,3-d]pyrimidine

The title compound was obtained as a yellow solid, 50 mg, 21%, from the title compound from step 4 and (S)-1-((S)-1-methylpyrrolidin-2-yl)ethan-1-ol, following a similar procedure to that described in Example 47 and 48, step 3. LCMS m/z=326 [M+H]+

Step 6: Synthesis of tert-butyl (4-(5-chloro-1-(8,8-dimethyl-4-((S)-1-((S)-1-methylpyrrolidin-2-yl)ethoxy)-7,8-dihydro-5H-pyrano[4,3-d]pyrimidin-2-yl)-1H-indazol-4-yl)-3-cyano-7-fluorobenzo[b]thiophen-2-yl)carbamate

A mixture of the title compound from step 5 (40 mg, 122 μmol), Intermediate 16 (54.0 mg, 122 μmol), CS₂CO₃ (79.3 mg, 244 μmol), Gphos (65.3 mg, 122 μmol) and Gphos Pd (11.0 mg, 12.2 μmol) in dioxane (5 mL) was stirred at 80° C. under N₂ for 4 h. The reaction mixture was diluted with EtOAc (100 mL) washed with water (100 mL×3) and saturated brine (100 mL). The organic layer was dried over Na₂SO₄, filtered and concentrated in vacuo. The crude product was purified by silica gel column with DCM:MeOH=20:1, to give the title compound (60 mg, 67.1%) as a yellow solid. LCMS m/z=732 [M+H]+

Step 7: Synthesis of (S)-2-amino-4-(5-chloro-1-(8,8-dimethyl-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile and (R)-2-amino-4-(5-chloro-1-(8,8-dimethyl-4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

The title compounds were obtained from the title compound from step 6, following a similar procedure to that described in Example 44 and 45, step 2.

Example 97, (6.0 mg, 11.6%) as a white solid. LCMS: m/z=632 [M+H]⁺; 1H NMR (400 MHz, DMSO-d₆) δ 8.69 (d, 1H), 8.03 (s, 2H), 7.94 (s, 1H), 7.78 (d, 1H), 7.27 (dd, 1H), 7.22-7.13 (m, 1H), 5.40 (t, 1H), 4.67 (m, 2H), 3.72 (s, 2H), 2.97 (m, 1H), 2.70 (s, 1H), 2.40 (s, 3H), 2.32 (s, 1H), 1.90 (s, 1H), 1.70 (m, 3H), 1.34 (dd, 9H).

Example 98 (6.0 mg, 11.6%) as an off-white solid. LCMS: m/z=632 [M+H]⁺; 1H NMR (400 MHz, DMSO-d₆) δ 8.70 (d, 1H), 8.03 (s, 2H), 7.94 (s, 1H), 7.78 (d, 1H), 7.27 (dd, 1H), 7.22-7.13 (m, 1H), 5.46-5.38 (m, 1H), 4.70-4.56 (m, 2H), 3.72 (s, 2H), 2.97 (m, 1H), 2.62 (d, 1H), 2.36 (s, 3H), 2.20 (d, 1H), 1.82 (d, 1H), 1.72 (m, 3H), 1.34 (dd, 9H).

### Example 99

### (S)-2-amino-4-(3,5-dichloro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (R)-2-amino-4-(3,5-dichloro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

### And Example 100

### (R)-2-amino-4-(3,5-dichloro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (S)-2-amino-4-(3,5-dichloro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

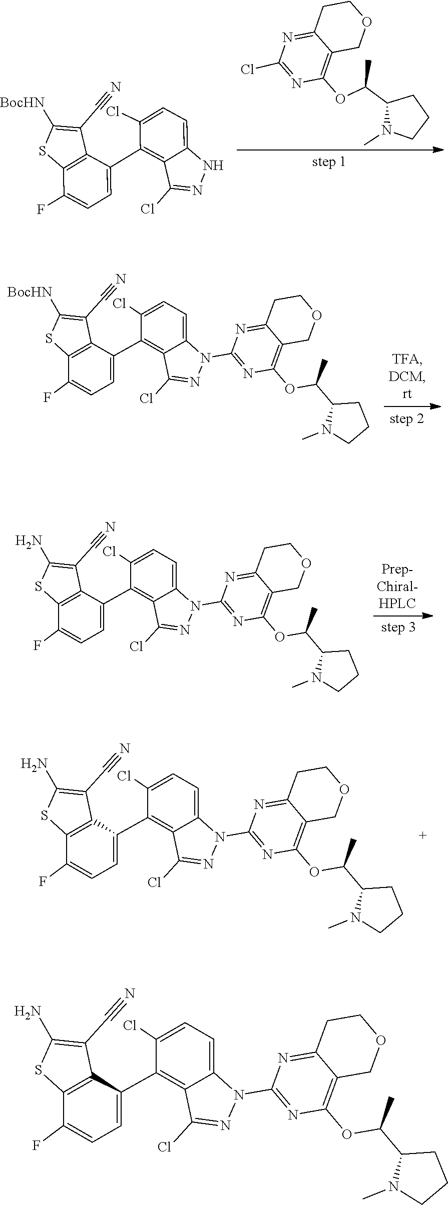

Step 1: Synthesis of tert-butyl (3-cyano-4-(3,5-dichloro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophen-2-yl)carbamate

To a stirred solution of Intermediate 17 (200 mg, 418 μmol) and Intermediate 1 (136 mg, 459 μmol) in dioxane (4 mL) were added Pd-PEPPSI-IHPentCl₃ (20.3 mg, 20.9 μmol) and Cs₂CO₃ (272 mg, 836 μmol) under N₂ and the reaction mixture was stirred for 12 h at 100° C. Water (10 mL) was added, the mixture was extracted with DCM (10 mL×3) and the combined organic layers were dried over Na₂SO₄ and concentrated. The crude product was purified by Prep-TLC with DCM:MeOH=10:1 to give the title compound (100 mg, 32%) as a yellow solid. LCMS m/z=738 [M+H]+

Step 2: Synthesis of 2-amino-4-(3,5-dichloro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

The title compound was obtained as a yellow solid, 40 mg, 46%, from the title compound of step 1, following a similar procedure to that described in Example 1 and 2, step 2. LCMS m/z=638 [M+H]+

Step 3: Synthesis of (S)-2-amino-4-(3,5-dichloro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile and (R)-2-amino-4-(3,5-dichloro-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

The title compound from step 2 was purified by Prep-Chiral-HPLC (Method B1, Gradient: 58% B to 83% B in 7 min) to give Peak 1, Example 99, (9.4 mg, 23%) as a white solid and Peak 2, Example 100, (8.8 mg, 22%) as a white solid.

Example 99, LCMS: m/z=638 [M+H]⁺, 1H NMR (400 MHz, DMSO-d₆) δ 8.76 (d, 1H), 8.04 (s, 2H), 7.84 (d, 1H), 7.26 (dd, 1H), 7.15 (dd, 1H), 5.41 (p, 1H), 4.67-4.53 (m, 2H), 4.01 (t, 2H), 3.02-2.91 (m, 4H), 2.43 (s, 3H), 2.43-2.40 (m, 1H), 1.88-1.72 (m, 4H), 1.35 (d, 3H).

Example 100, LCMS: m/z=638 [M+H]⁺, 1H NMR (400 MHz, DMSO-d₆) δ 8.77 (d, 1H), 8.04 (s, 2H), 7.84 (d, 1H), 7.26 (dd, 1H), 7.16 (dd, 1H), 5.43 (p, 1H), 4.68-4.54 (m, 2H), 4.02 (t, 2H), 3.02-2.91 (m, 4H), 2.43 (s, 3H), 2.43-2.40 (m, 1H), 1.89-1.67 (m, 4H), 1.37 (d, 3H).

### Example 101

### (S)-2-amino-4-(5-chloro-1-(4-((S)-1-((S)-1-methylpyrrolidin-2-yl)ethoxy)-5,6,8,9-tetrahydrooxepino[4,5-d]pyrimidin-2-yl)-1H-indazol-4-yl)-7-fluorobenzo[b]thiophene-3-carbonitrile or (R)-2-amino-4-(5-chloro-1-(4-((S)-1-((S)-1-methylpyrrolidin-2-yl)ethoxy)-5,6,8,9-tetrahydrooxepino[4,5-d]pyrimidin-2-yl)-1H-indazol-4-yl)-7-fluorobenzo[b]thiophene-3-carbonitrile

### And Example 102

### (R)-2-amino-4-(5-chloro-1-(4-((S)-1-((S)-1-methylpyrrolidin-2-yl)ethoxy)-5,6,8,9-tetrahydrooxepino[4,5-d]pyrimidin-2-yl)-1H-indazol-4-yl)-7-fluorobenzo[b]thiophene-3-carbonitrile or (S)-2-amino-4-(5-chloro-1-(4-((S)-1-((S)-1-methylpyrrolidin-2-yl)ethoxy)-5,6,8,9-tetrahydrooxepino[4,5-d]pyrimidin-2-yl)-1H-indazol-4-yl)-7-fluorobenzo[b]thiophene-3-carbonitrile

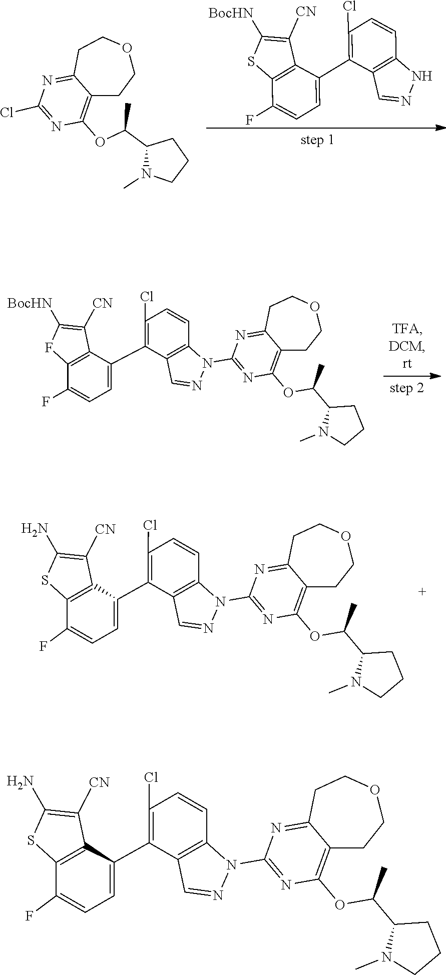

Step 1: Synthesis of tert-butyl (4-(5-chloro-1-(4-((S)-1-((S)-1-methylpyrrolidin-2-yl)ethoxy)-5,6,8,9-tetrahydrooxepino[4,5-d]pyrimidin-2-yl)-1H-indazol-4-yl)-3-cyano-7-fluorobenzo[b]thiophen-2-yl)carbamate

A solution of Intermediate 16 (270 mg, 609 μmol), the compound of Example 25 and 26, step 1 (284 mg, 913 μmol), t-BuBrettphos Pd G3 (103 mg, 121 μmol), t-BuBrettphos (58.5 mg, 121 μmol) and Cs₂CO₃ (394 mg, 1.21 mmol) in dioxane (5 mL) was stirred at 100° C. for 2 h. The cooled mixture was extracted with EtOAc (100 mL×3), the combined organic layers washed with brine (50 mL×2), dried with Na₂SO₄ and concentrated in vacuo. The crude product was purified by silica gel column chromatography with DCM:MeOH=10:1, to give the title compound, 350 mg, 77.2% as a yellow solid. LCMS m/z=719 [M+H]+

Step 2: Synthesis of (S)-2-amino-4-(5-chloro-1-(4-((S)-1-((S)-1-methylpyrrolidin-2-yl)ethoxy)-5,6,8,9-tetrahydrooxepino[4,5-d]pyrimidin-2-yl)-1H-indazol-4-yl)-7-fluorobenzo[b]thiophene-3-carbonitrile and (R)-2-amino-4-(5-chloro-1-(4-((S)-1-((S)-1-methylpyrrolidin-2-yl)ethoxy)-5,6,8,9-tetrahydrooxepino[4,5-d]pyrimidin-2-yl)-1H-indazol-4-yl)-7-fluorobenzo[b]thiophene-3-carbonitrile

To a solution of the title compound of step 1 (340 mg, 472.6 μmol) in DCM (4.2 mL) was added TFA (1.4 mL) at 0° C. and the reaction mixture was stirred at 25° C. for 2 h. The mixture was concentrated in vacuo and the residue was purified by prep HPLC (Method A2, Gradient: 9% B to 39% B in 18 min), to give Peak 1, Example 101, 56.5 mg (59.7%) as a white solid and Peak 2, Example 102, 33.9 mg (38.8%) as a white solid.

Example 101, LCMS: m/z=618 [M+H]⁺, 1H NMR (400 MHz, DMSO-d₆) δ 8.71 (dd, 1H), 8.02 (s, 2H), 7.91 (d, 1H), 7.71 (d, 1H), 7.28 (dd, 1H), 7.17 (dd, 1H), 5.41 (p, 1H), 3.81 (dd, 2H), 3.72 (dd, 2H), 3.21 (dd, 2H), 3.01-2.92 (m, 3H), 2.62 (d, 1H), 2.36 (s, 3H), 2.21 (d, 1H), 1.90-1.64 (m, 4H), 1.33 (d, 3H).

Example 102, LCMS: m/z=618 [M+H]+, 1H NMR (400 MHz, DMSO-d₆) δ 8.72 (d, 1H), 8.03 (s, 2H), 7.91 (s, 1H), 7.72 (d, 1H), 7.28 (dd, 1H), 7.17 (dd, 1H), 5.42 (p, 1H), 3.88-3.64 (m, 4H), 3.21 (q, 2H), 3.03-2.92 (m, 3H), 2.69 (dd, 1H), 2.39 (s, 3H), 2.25 (td, 1H), 1.93-1.63 (m, 4H), 1.34 (d, 3H).

### Example 103

### (R)-2-amino-5-chloro-7-fluoro-4-(1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophene-3-carbonitrile or (S)-2-amino-5-chloro-7-fluoro-4-(1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophene-3-carbonitrile

### And Example 104

### (S)-2-amino-5-chloro-7-fluoro-4-(1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophene-3-carbonitrile or (R)-2-amino-5-chloro-7-fluoro-4-(1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophene-3-carbonitrile

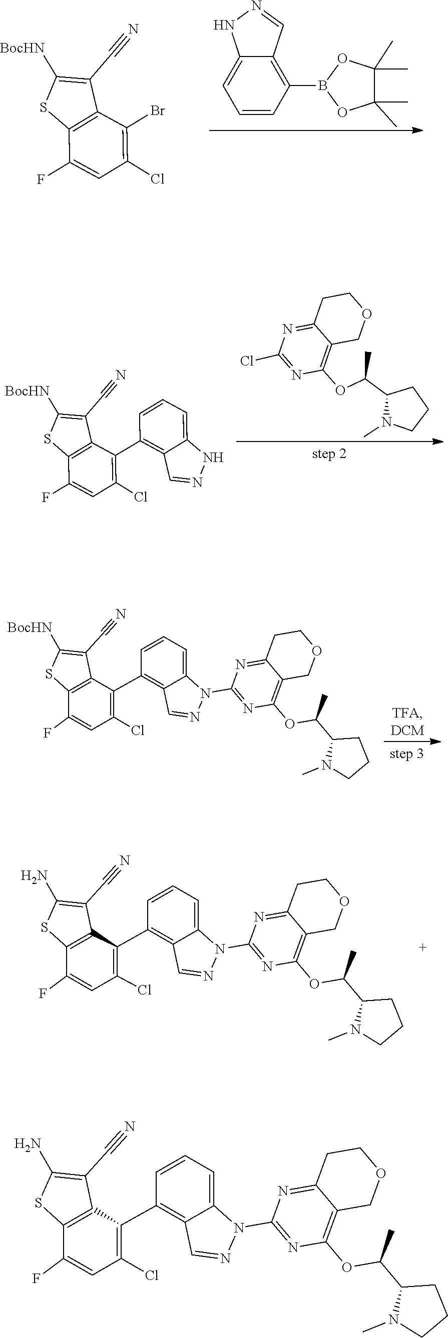

Step 1: Synthesis of tert-butyl (5-chloro-3-cyano-7-fluoro-4-(1H-indazol-4-yl)benzo[b]thiophen-2-yl)carbamate

The title compound was obtained as a yellow solid, 160 mg, 73%, from Intermediate 8 and 4-(4,4,5,5-tetramethyl-1,3,2-dioxaborolan-2-yl)-1H-indazole, following a similar procedure to that described in Example 42, step 1. LCMS m/z=443 [M+H]⁺

Step 2: Synthesis of tert-butyl (5-chloro-3-cyano-7-fluoro-4-(1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophen-2-yl)carbamate

To a stirred solution of the title compound from step 1 (120 mg, 270 μmol) and Intermediate 1 (176 mg, 590 μmol) in DMF (3 mL) was added Cs₂CO₃ (105.5 mg, 324 μmol) and the reaction was stirred for 2 h at 100° C. Water (10 mL) was added to the cooled reaction and the mixture was extracted with DCM (10 mL×3). The combined organic layers were washed with water (10 mL×2) and brine (10 mL), then dried over Na₂SO₄ and concentrated. The crude product was purified by Prep-TLC with DCM:MeOH=10:1 to give the title compound (70 mg, 36%) as a yellow solid. LCMS m/z=705 [M+H]+

Step 3: Synthesis of (R)-2-amino-5-chloro-7-fluoro-4-(1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophene-3-carbonitrile and (S)-2-amino-5-chloro-7-fluoro-4-(1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophene-3-carbonitrile

The title compounds were obtained from the title compound from step 2, following a similar procedure to that described in Example 77 and 78 step 3.

Example 103 mg, 2 mg, 7% as a white solid. LCMS: m/z=604 [M+H]⁺, 1H NMR (400 MHz, DMSO-d₆) δ 8.73 (d, 1H), 8.08 (s, 2H), 7.99 (s, 1H), 7.65 (t, 1H), 7.44 (d, 1H), 7.25 (d, 1H), 5.51-5.44 (m, 1H), 4.68-4.54 (m, 2H), 4.01 (d, 2H), 3.06-3.03 (m, 1H), 2.93-2.80 (m, 3H), 2.45 (s, 3H), 2.35-2.33 (m, 1H), 1.91-1.74 (m, 4H), 1.36 (d, 3H).

Example 104, 1.3 mg, 4% as a white solid. LCMS: m/z=604 [M+H]⁺, 1H NMR (400 MHz, DMSO-d₆) δ 8.74 (d, 1H), 8.08 (s, 2H), 7.98 (s, 1H), 7.65 (t, 1H), 7.44 (d, 1H), 7.24 (d, 1H), 5.51-5.48 (m, 1H), 4.68-4.54 (m, 2H), 4.01 (s, 2H), 2.97-2.92 (m, 4H), 2.39 (s, 3H), 1.85-1.66 (m, 5H), 1.35 (d, 3H).

### Examples 105 to 107

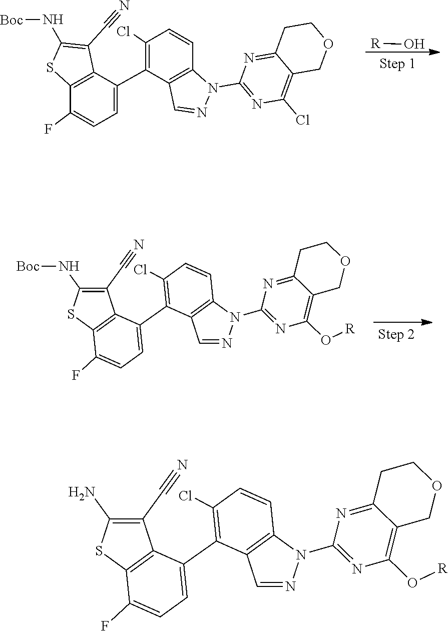

### Step 1:

KOtBu (22.40 mg, 0.20 mmol) was added to a solution of Intermediate 23 (61.1 mg, 0.10 mmol) and R—OH (3 eq) in THF (1.50 mL) and the reaction mixture was shaken at 80° C. for 16 h. The residue was diluted with H₂O (2.0 mL) and extracted with THF (2.0 mL×3).

The combined organic extracts were dried over anhydrous Na₂SO₄, filtered and concentrated under reduced pressure to give crude product.

### Step 2:

A solution of the product from step 1 (1 eq) and TFA (0.5 mL) in DCM (1.5 mL) was shaken at 30° C. for 2 h. The mixture was concentrated in vacuo and the residue purified by prep. HPLC to provide the title compounds.

| Example | | |
|---|---|---|
| No. | Structure, Name | Data |
| 105 | 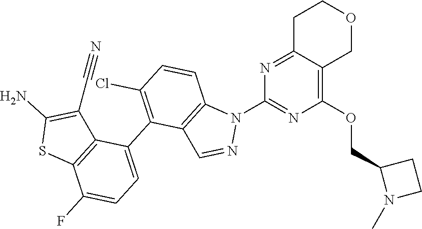 [SMILES: CN1CC[C@@H]1COc1nc(-n2ncc3c(-c4ccc(F)c5sc(N)c(C#N)c45)c(Cl)ccc32)nc2c1COCC2] | LCMS m/z = 577 [M + H]+ |
| | 2-amino-4-(5-chloro-1-(4-(((R)-1-methylazetidin- | |
| | 2-yl)methoxy)-7,8-dihydro-5H-pyrano[4,3- | |
| | d]pyrimidin-2-yl)-1H-indazol-4-yl)-7- | |
| | fluorobenzo[b]thiophene-3-carbonitrile | |
| 106 | 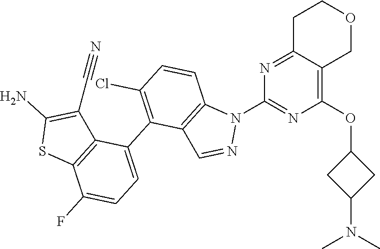 [SMILES: CN(C)C1CC(Oc2nc(-n3ncc4c(-c5ccc(F)c6sc(N)c(C#N)c56)c(Cl)ccc43)nc3c2COCC3)C1] | LCMS m/z = 590 [M + H]+ |
| | 2-amino-4-(5-chloro-1-(4-(3- | |
| | (dimethylamino)cyclobutoxy)-7,8-dihydro-5H- | |
| | pyrano[4,3-d]pyrimidin-2-yl)-1H-indazol-4-yl)-7- | |
| | fluorobenzo[b]thiophene-3-carbonitrile | |
| 107 | 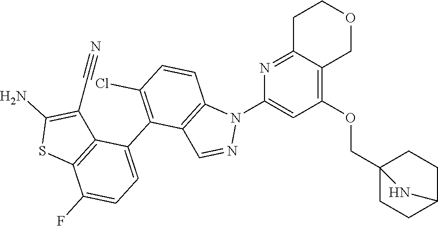 [SMILES: N#Cc1c(N)sc2c(F)ccc(-c3c(Cl)ccc4c3cnn4-c3cc(OCC45CCC(CC4)N5)c4c(n3)CCOC4)c12] | LCMS m/z = 602 [M + H]+ |
| | 4-(1-(4-((7-azabicyclo[2.2.1]heptan-1- | |
| | yl)methoxy)-7,8-dihydro-5H-pyrano[4,3- | |
| | d]pyrimidin-2-yl)-5-chloro-1H-indazol-4-yl)-2- | |
| | amino-7-fluorobenzo[b]thiophene-3-carbonitrile | |

### Examples 108 to 112

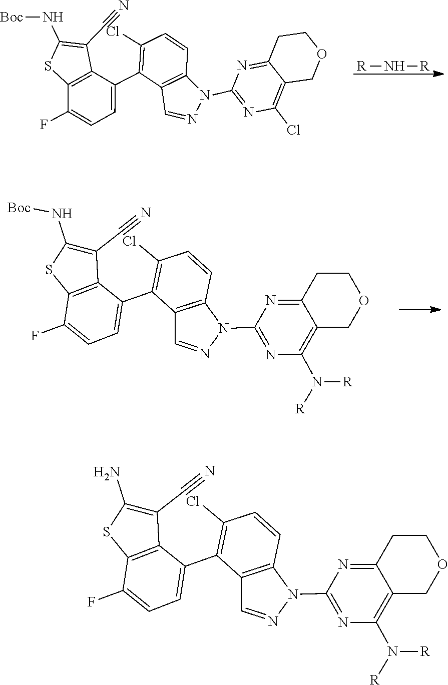

### Step 1:

Intermediate 23 (61.08 mg, 0.10 mmol, 1.0 eq) and R₂NH (3.0 eq) in DMSO (1.50 mL) were added DIEA (3.0 eq) and CsF (2.0 eq) and the reaction mixture was shaken at 100° C. for 16 h. The residue was diluted with H₂O (2.0 mL) and extracted with EtOAc (2.0 mL×3). The combined organic extracts were dried over anhydrous Na₂SO₄, filtered and concentrated under reduced pressure to give the crude product.

### Step 2:

The crude product from step 1 (1.0 eq) was dissolved in DCM (1.50 mL) and TFA (0.50 mL) and the reaction mixture was shaken at 30° C. for 2 h. The mixture was concentrated in vacuo and the residue was purified by prep HPLC (Xtimate C18 150*25 mm*5 μm, mobile phase A: NH₄OH/H₂O=0.05% v/v; mobile phase B: MeCN at appropriate gradient at 25 or 30 mL/min) to give the title compound.

| Example | | |
|---|---|---|
| No. | Structure, Name | Data |
| 108 | 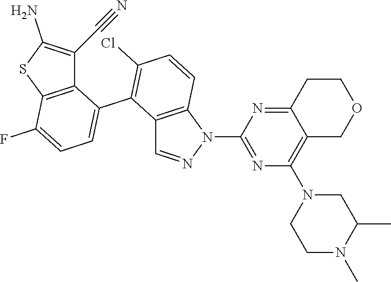 [SMILES: CC1CN(c2nc(-n3ncc4c(-c5ccc(F)c6sc(N)c(C#N)c56)c(Cl)ccc43)nc3c2COCC3)CCN1C] | LCMS: m/z = 589 [M + H]⁺ |
| | 2-amino-4-(5-chloro-1-(4-(3,4-dimethylpiperazin-1- | |
| | yl)-7,8-dihydro-5H-pyrano[4,3-d]pyrimidin-2-yl)-1H- | |
| | indazol-4-yl)-7-fluorobenzo[b]thiophene-3- | |
| | carbonitrile | |
| 109 | 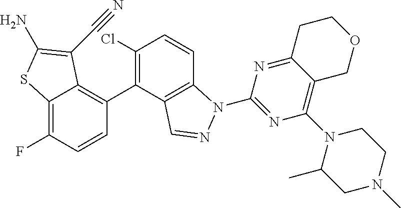 [SMILES: CC1CN(C)CCN1c1nc(-n2ncc3c(-c4ccc(F)c5sc(N)c(C#N)c45)c(Cl)ccc32)nc2c1COCC2] | LCMS: m/z = 589 [M + H]⁺ |
| | 2-amino-4-(5-chloro-1-(4-(2,4-dimethylpiperazin-1- | |
| | yl)-7,8-dihydro-5H-pyrano[4,3-d]pyrimidin-2-yl)-1H- | |
| | indazol-4-yl)-7-fluorobenzo[b]thiophene-3- | |
| | carbonitrile | |
| 110 | 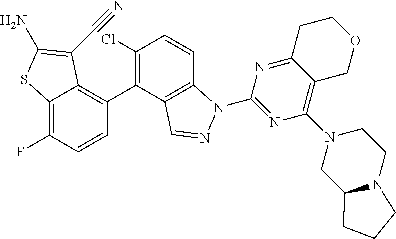 [SMILES: N#Cc1c(N)sc2c(F)ccc(-c3c(Cl)ccc4c3cnn4-c3nc4c(c(N5CCN6CCC[C@H]6C5)n3)COCC4)c12] | LCMS: m/z = 601 [M + H]⁺ |
| | 2-amino-4-(5-chloro-1-(4-((S)-hexahydropyrrolo[1,2- | |
| | a]pyrazin-2(1H)-yl)-7,8-dihydro-5H-pyrano[4,3- | |
| | d]pyrimidin-2-yl)-1H-indazol-4-yl)-7- | |
| | fluorobenzo[b]thiophene-3-carbonitrile | |
| 111 | 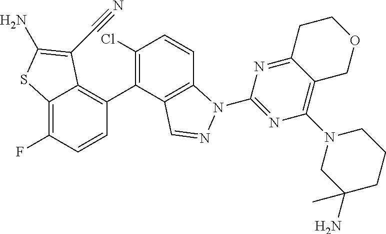 [SMILES: CC1(N)CCCN(c2nc(-n3ncc4c(-c5ccc(F)c6sc(N)c(C#N)c56)c(Cl)ccc43)nc3c2COCC3)C1] | LCMS: m/z = 589 [M + H]⁺ |
| | 2-amino-4-(1-(4-(3-amino-3-methylpiperidin-1-yl)- | |
| | 7,8-dihydro-5H-pyrano[4,3-d]pyrimidin-2-yl)-5- | |
| | chloro-1H-indazol-4-yl)-7-fluorobenzo[b]thiophene-3- | |
| | carbonitrile | |
| 112 | 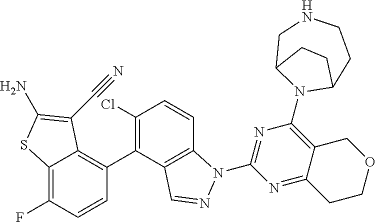 [SMILES: N#Cc1c(N)sc2c(F)ccc(-c3c(Cl)ccc4c3cnn4-c3nc4c(c(N5C6CCNCC5CC6)n3)COCC4)c12] | LCMS: m/z = 601 [M + H]⁺ |
| | 4-(1-(4-(3,9-diazabicyclo[4.2.1]nonan-9-yl)-7,8- | |
| | dihydro-5H-pyrano[4,3-d]pyrimidin-2-yl)-5-chloro- | |
| | 1H-indazol-4-yl)-2-amino-7-fluorobenzo[b]thiophene- | |
| | 3-carbonitrile | |

### Example 113

### (R)-2-amino-4-(5-chloro-3-fluoro-6-methyl-1-(4-((S)-1-((S)-1-methylpyrrolidin-2-yl)ethoxy)-6,7-dihydro-9H-[1,4]dioxepino[6,5-d]pyrimidin-2-yl)-1H-indazol -4-yl)-7-fluorobenzo[b]thiophene-3-carbonitrile

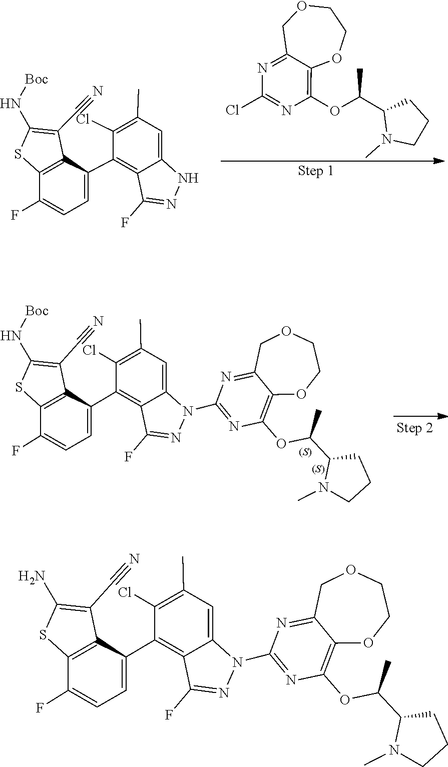

Step 1: Synthesis of tert-butyl ((R)-4-(5-chloro-3-fluoro-6-methyl-1-(4-((S)-1-((S)-1-methylpyrrolidin-2-yl)ethoxy)-6,7-dihydro-9H-[1,4]dioxepino[6,5-d]pyrimidin-2-yl)-1H-indazol-4-yl)-3-cyano-7-fluorobenzo[b]thiophen-2-yl)carbamate:

A solution of Intermediate 25 (5.80 g, 12.2 mmol), Intermediate 26 (3.93 g, 12.5 mmol), Cs₂CO₃ (7.96 g, 24.4 mmol) and EPhos Pd G4 (600 mg, 653 μmol) in dioxane (60 mL) was stirred at 80° C. for 1 h. The cooled reaction mixture was concentrated to give a residue and was purified by silica gel column chromatography (petroleum ether:EtOAc=1:0 to EtOAc: methanol=10:1) to give the title compound (9.50 g, 99% yield) as a yellow solid. LCMS m/z=752.2 [M+H]+

Step 2: Synthesis of (R)-2-amino-4-(5-chloro-3-fluoro-6-methyl-1-(4-((S)-1-((S)-1-methylpyrrolidin-2-yl)ethoxy)-6,7-dihydro-9H-[1,4]dioxepino[6,5-d]pyrimidin-2-yl)-1H-indazol-4-yl)-7-fluorobenzo[b]thiophene-3-carbonitrile

To a solution of the title compound from step 1 (9.50 g, 12.6 mmol) in dioxane (33 mL) was added HCl (6 N aqueous, 4 mL) and the reaction mixture was stirred at 50° C. for 3 h. The reaction mixture was treated with aqueous saturated NaHCO₃ and extracted with EtOAc (400 mL×3). The combined organic layers were dried over Na₂SO₄, filtered and concentrated in vacuo. The residue was purified by prep-HPLC (Kromasil Eternity XT 250*70 mm*10 um; mobile phase: [H2O (0.05% NH3H2O)-ACN]; gradient:60%-90% B over 20.0 min) to provide the title compound (5.15 g, 62%) as a yellow solid. LCMS m/z=652.2 [M+H]+, ¹H NMR (400 MHz, CD₃OD) δ ppm 8.70 (s, 1H), 7.27-7.21 (m, 1H), 7.07-7.00 (m, 1H), 5.41-5.32 (m, 1H), 4.84 (s, 2H), 4.22-4.15 (m, 2H), 4.09-4.02 (m, 2H), 3.10-3.04 (m, 1H), 2.81-2.74 (m, 1H), 2.63 (s, 3H), 2.57 (s, 3H), 2.43-2.36 (m, 1H), 2.17-2.06 (m, 1H), 1.87-1.71 (m, 3H), 1.50 (d, 3H).

### Example 114

### (R)-2-amino-4-(5-chloro-1-(4-((S)-1-((2S,4R)-1,4-dimethylpyrrolidin-2-yl)ethoxy)-6,7-dihydro-9H-[1,4]dioxepino[6,5-d]pyrimidin-2-yl)-3-fluoro-6-methyl-1H-indazol -4-yl)-7-fluorobenzo[b]thiophene-3-carbonitrile

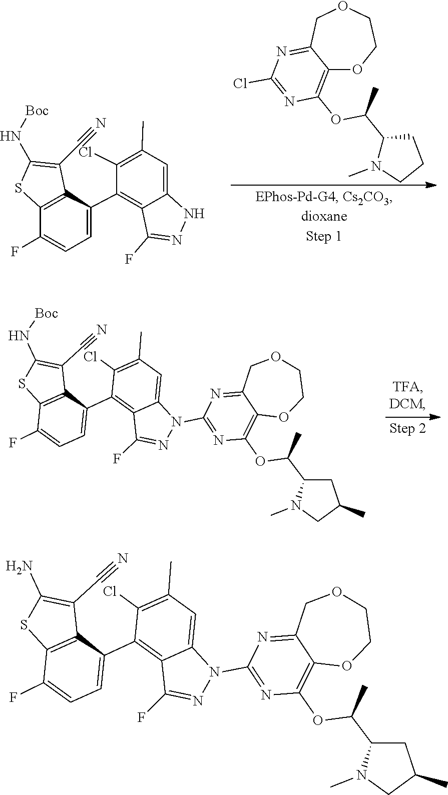

Step 1: Synthesis of tert-butyl ((R)-4-(5-chloro-1-(4-((S)-1-((2S,4R)-1,4-dimethylpyrrolidin-2-yl)ethoxy)-6,7-dihydro-9H-[1,4]dioxepino[6,5-d]pyrimidin-2-yl)-3-fluoro-6-methyl-1H-indazol-4-yl)-3-cyano-7-fluorobenzo[b]thiophen-2-yl)carbamate

A solution of Intermediate 25 (130 mg, 274 μmol), Intermediate 28 (90.0 mg, 274 μmol), Cs₂CO₃ (177 mg, 549 μmol) and EPhos Pd G4 (25.2 mg, 27.4 μmol) in dioxane (2.0 mL) was stirred at 80° C. for 0.5 h. The cooled reaction mixture was concentrated to give a residue and was purified by prep-HPLC (Method A4, gradient:36%-66% B over 10.0 min) to give the title compound (75.0 mg, 97.8 μmol, 35% yield) as a yellow solid. LCMS m/z=766.3 [M+H]+

Step 2: Synthesis of (R)-2-amino-4-(5-chloro-1-(4-((S)-1-((2S,4R)-1,4-dimethylpyrrolidin-2-yl)ethoxy)-6,7-dihydro-9H-[1,4]dioxepino[6,5-d]pyrimidin-2-yl)-3-fluoro-6-methyl-1H-indazol-4-yl)-7-fluorobenzo[b]thiophene-3-carbonitrile

To a solution of the title compound from step 1 (70.0 mg, 91.35 μmol) in DCM (2.1 mL) was added TFA (1.07 g, 9.42 mmol, 700 μL) and the reaction mixture was stirred at rt for 0.5 h. The reaction mixture was then concentrated in vacuo and the residue was purified by prep-HPLC (Method A4, gradient:28%-58% B over 15.0 min) to give the title compound (39.3 mg, 50.4 μmol, 55% yield, TFA) as an off-white solid. LCMS m/z=666.4 [M+H]+ ¹H NMR (400 MHz, 6d-DMSO): δ ppm 9.86 (br s, 1H), 8.70 (s, 1H), 8.07 (s, 2H), 7.36-7.28 (m, 1H), 7.22-7.10 (m, 1H), 5.34-5.21 (m, 1H), 5.00-4.87 (m, 2H), 4.34-4.24 (m, 1H), 4.23-4.15 (m, 1H), 4.13-4.02 (m, 2H), 4.01-3.91 (m, 1H), 3.65-3.59 (m, 1H), 3.09 (d, 3H), 2.89-2.77 (m, 1H), 2.62 (s, 3H), 2.40-2.31 (m, 1H), 2.19-2.08 (m, 1H), 2.00-1.88 (m, 1H), 1.55 (d, 3H), 1.07 (d, 3H).

### Examples 115, 28a, 12a, and 117 to 122

The compounds in the following table were prepared in a 2-step process, from Intermediate 25 and the appropriate chloride starting material (SM) using a suitable catalyst in step 1 and suitable deprotection method in step 2 following a similar procedure to that described above.

| Example | Step 1 Product, Starting Materials (SM), | Step 2 Product, Purification Method, |
|---|---|---|
| No | Catalyst, Data | Data |
| 115 | tert-butyl ((R)-4-(5-chloro-3-fluoro-6- | (R)-2-amino-4-(5-chloro-3-fluoro-6- |
| | methyl-1-(4-((S)-1-((S)-1- | methyl-1-(4-((S)-1-((S)-1- |
| | methylpyrrolidin-2-yl)ethoxy)-7-oxo- | methylpyrrolidin-2-yl)ethoxy)-7-oxo- |
| | 6,7,8,9-tetrahydro-5H-pyrimido[4,5- | 6,7,8,9-tetrahydro-5H-pyrimido[4,5- |
| | c]azepin-2-yl)-1H-indazol-4-yl)-3- | c]azepin-2-yl)-1H-indazol-4-yl)-7- |
| | cyano-7-fluorobenzo[b]thiophen-2- | fluorobenzo[b]thiophene-3- |
| | yl)carbamate | carbonitrile |
| | 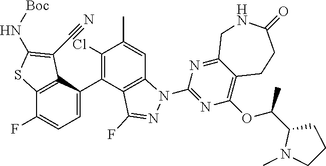 | 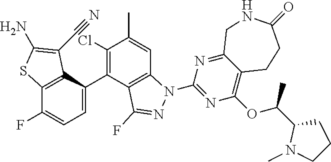 |
| | SM: Intermediate 27 | Method A4. Gradient: 18-48% B in |
| | Catalyst: 0.1 eq. Ephos Pd G4 | 15 min. |
| | LCMS: m/z = 764 [M + H]⁺ | LCMS: m/z = 663.4 [M + H]⁺ |
| | ¹H NMR (400 MHz, CD₃OD): δ | ¹H NMR (400 MHz, DMSO-d6) δ = |
| | 8.77 (s, 1H), 7.39 (dd, 1H), 7.27- | 9.91-9.77 (m, 1H), 8.73 (s, 1H), 8.11- |
| | 7.18 (m, 1H), 5.58-5.47 (m, 1H), | 7.99 (m, 3H), 7.35-7.27 (m, 1H), 7.20- |
| | 4.61-4.56 (m, 2H), 3.24-3.14 | 7.11 (m, 1H), 5.45-5.32 (m, 1H), 4.74- |
| | (m, 1H), 3.06-2.86 (m, 5H), | 4.62 (m, 1H), 4.51-4.42 (m, 1H), 3.86- |
| | 2.65 (s, 3H), 2.60 (s, 3H), | 3.75 (m, 1H), 3.61-3.51 (m, 1H), 3.30- |
| | 2.57-2.50 (m, 1H), 2.22- | 3.14 (m, 1H), 3.00-2.98 (m, 3H), 2.96- |
| | 2.07 (m, 1H), 1.96-1.77 (m, | 2.87 (m, 2H), 2.86-2.71 (m, 2H), 2.62 |
| | 3H), 1.56 (s, 9H), 1.48 | (s, 3H), 2.20-2.08 (m, 1H), 2.08-1.77 |
| | (d, 3H) | (m, 3H), 1.49 (d, 3H). |
| 28a | tert-butyl ((R)-4-(5-chloro-3-fluoro-6- | (R)-2-amino-4-(5-chloro-3-fluoro-6- |
| | methyl-1-(4-((S)-1-((S)-1- | methyl-1-(4-((S)-1-((S)-1- |
| | methylpyrrolidin-2-yl)ethoxy)-7,8- | methylpyrrolidin-2-yl)ethoxy)-7,8- |
| | dihydro-5H-pyrano[4,3-d]pyrimidin-2- | dihydro-5H-pyrano[4,3-d]pyrimidin-2- |
| | yl)-1H-indazol-4-yl)-3-cyano-7- | yl)-1H-indazol-4-yl)-7- |
| | fluorobenzo[b]thiophen-2-yl)carbamate | fluorobenzo[b]thiophene-3-carbonitrile |
| | 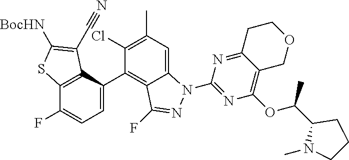 | 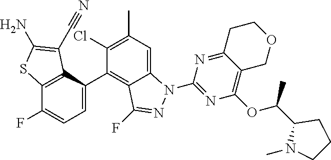 |
| | SM: Intermediate 1 | Method A4. Gradient: 23-53% B in 15 min. |
| | Catalyst: 0.1 eq. Ephos Pd G4 | LCMS: m/z = 636.3 [M + H]⁺. |
| | LCMS: m/z = 736.2 [M + H]⁺. | ¹H NMR (400 MHz, DMSO-d₆) δ = 9.99 (br |
| | | s, 1H), 8.72 (s, 1H), 8.06 (s, 2H), 7.31 (dd, |
| | | 1H), 7.16 (t, 1H), 5.46-5.36 (m, 1H), 4.73- |
| | | 4.57 (m, 2H), 4.03 (qd, 2H), 3.78 (br dd, |
| | | 2H), 3.59-3.53 (m, 1H), 3.17 (br dd, 1H), |
| | | 3.02-2.99 (m, 1H), 2.96 (br d, 3H), 2.60 (s, |
| | | 3H), 2.36-2.22 (m, 1H), 2.14-2.04 (m, |
| | | 1H), 1.99-1.83 (m, 2H), 1.50 (d, 3H) |
| 12a | tert-butyl ((R)-4-(5-chloro-3-fluoro-6- | (R)-2-amino-4-(5-chloro-3-fluoro-6- |
| | methyl-1-(4-((S)-1-((S)-1- | methyl-1-(4-((S)-1-((S)-1- |
| | methylpyrrolidin-2-yl)ethoxy)-5,7- | methylpyrrolidin-2-yl)ethoxy)-5,7- |
| | dihydrofuro[3,4-d]pyrimidin-2-yl)-1H- | dihydrofuro[3,4-d]pyrimidin-2-yl)-1H- |
| | indazol-4-yl)-3-cyano-7- | indazol-4-yl)-7- |
| | fluorobenzo[b]thiophen-2-yl)carbamate | fluorobenzo[b]thiophene-3-carbonitrile |
| | 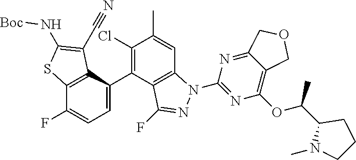 | 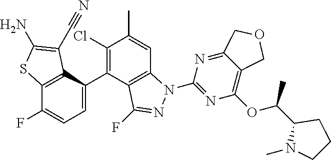 |
| | SM: Intermediate 2 | prep-HPLC column: Phenomenex Luna C18 |
| | Catalyst: 0.1 eq. Ephos Pd G4 | 150*25 mm* 10 um; mobile phase: [H2O |
| | LCMS m/z = 722.3 [M + H]⁺. | (0.225% FA)-ACN]; gradient: 25%-55% B |
| | | over 10.0 min |
| | | LCMS m/z = 622.2 [M + H]⁺. |
| | | 1H NMR (400 MHz, DMSO-d6) δ = 8.70 (s, |
| | | 1H), 8.14 (s, 1H), 8.06 (s, 2H), 7.37-7.28 |
| | | (m, 1H), 7.16 (t, 1H), 5.47 (t, 1H), 5.07 (br |
| | | s, 4H), 3.15-3.00 (m, 1H), 2.60 (s, 3H), 2.51 |
| | | (s, 5H), 2.02-1.89 (m, 1H), 1.76 (br s, 3H), |
| | | 1.42 (br d, 3H) |
| 117 | tert-butyl ((R)-4-(5-chloro-3-fluoro-6- | (R)-2-amino-4-(5-chloro-3-fluoro-6- |
| | methyl-1-(4′-((S)-1-((S)-1- | methyl-1-(4′-((S)-1-((S)-1- |
| | methylpyrrolidin-2-yl)ethoxy)-2,3,5,6- | methylpyrrolidin-2-yl)ethoxy)-2,3,5,6- |
| | tetrahydro-5′H,7′H-spiro[pyran-4,8′- | tetrahydro-5′H,7′H-spiro[pyran-4,8′- |
| | pyrano[4,3-d]pyrimidin]-2′-yl)-1H- | pyrano[4,3-d]pyrimidin]-2′-yl)-1H- |
| | indazol-4-yl)-3-cyano-7- | indazol-4-yl)-7- |
| | fluorobenzo[b]thiophen-2-yl)carbamate | fluorobenzo[b]thiophene-3-carbonitrile |
| | 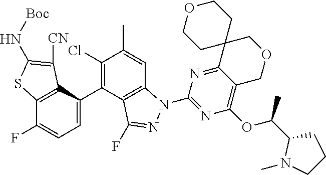 | 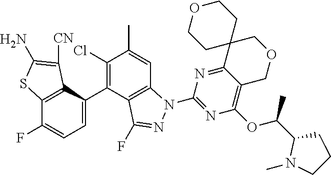 |
| | SM: Intermediate 29 | Method A4. Gradient: 30-60% B in |
| | Catalyst: 0.1 eq. Ephos Pd G4 | 10 min. |
| | LCMS m/z = 806.2 [M +H]⁺. | LCMS m/z = 706.3 [M + H]. |
| | | 1H NMR (400 MHz, DMSO-d6) δ = 8.72 |
| | | (s, 1H), 8.06 (s, 2H), 7.35-7.27 (m, 1H), |
| | | 7.16 (t, 1H), 5.49-5.28 (m, 1H), 4.78-4.54 |
| | | (m, 2H), 4.16-3.99 (m, 2H), 3.96-3.85 (m, |
| | | 2H), 3.62 (t, 2H), 3.02-2.64 (m, 3H), 2.60 |
| | | (s, 3H), 2.50 (s, 3H), 2.34-2.23 (m, 2H), |
| | | 2.22-2.04 (m, 1H), 2.01-1.74 (m, 3H), |
| | | 1.60 (d, 2H), 1.44 (d, 3H). |
| 118 | tert-butyl ((R)-4-(5-chloro-1-(4-((R)-1- | (R)-2-amino-4-(5-chloro-1-(4-((S)-1- |
| | ((R)-1-ethylpyrrolidin-2-yl)ethoxy)-7,8- | ((S)-1-ethylpyrrolidin-2-yl)ethoxy)-7,8- |
| | dihydro-5H-pyrano[4,3-d]pyrimidin-2- | dihydro-5H-pyrano[4,3-d]pyrimidin-2- |
| | yl)-3-fluoro-6-methyl-1H-indazol-4-yl)- | yl)-3-fluoro-6-methyl-1H-indazol-4-yl)- |
| | 3-cyano-7-fluorobenzo[b]thiophen-2- | 7-fluorobenzo[b]thiophene-3- |
| | yl)carbamate | carbonitrile |
| | 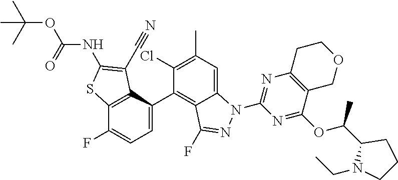 | 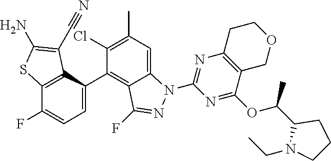 |
| | SM: Intermediate 30 | prep HPLC, ISCO XBridge 30 × 250 mm |
| | Catalyst: 0.1 eq. Ephos Pd G4 | column, 0.1% TFA, 42.5 mL/min, 10 to |
| | LCMS m/z = 750.5 [M + H]⁺. | 55% MeCN/H₂O, 25 min. |
| | | LCMS: m/z = 650.2 [M + H]⁺ |
| | | ¹H NMR (500 MHz, DMSO) δ 8.73 (s, |
| | | 1H), 8.05 (s, 2H), 7.32 (dd, 1H), 7.16 |
| | | (dd, 1H), 5.42 (p, 1H), 4.70-4.45 (m, |
| | | 2H), 4.01 (t, 2H), 3.05 (t, 1H), 2.99-2.79 |
| | | (m, 4H), 2.60 (s, 3H), 2.44-2.31 (m, |
| | | 1H), 2.24-2.15 (m, 1H), 1.88-1.58 (m, |
| | | 4H), 1.36 (d, 3H), 1.01 (t, 3H). |
| 119 | tert-butyl ((R)-4-(5-chloro-3-fluoro-6- | ((R)-2-amino-4-(5-chloro-3-fluoro-6- |
| | methyl-1-((R)-7-methyl-4-((S)-1-((S)-1- | methyl-1-((R)-7-methyl-4-((S)-1-((S)-1- |
| | methylpyrrolidin-2-yl)ethoxy)-6,7- | methylpyrrolidin-2-yl)ethoxy)-6,7- |
| | dihydro-9H-[1,4]dioxepino[6,5- | dihydro-9H-[1,4]dioxepino[6,5- |
| | d]pyrimidin-2-yl)-1H-indazol-4-yl)-3- | d]pyrimidin-2-yl)-1H-indazol-4-yl)-7- |
| | cyano-7-fluorobenzo[b]thiophen-2- | fluorobenzo[b]thiophene-3- |
| | yl)carbamate | carbonitrile |
| | 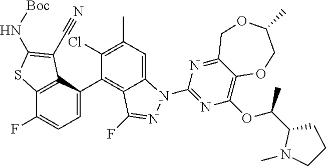 | 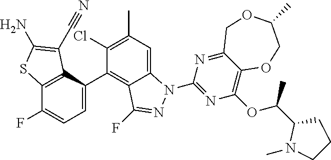 |
| | SM: Example 34 | Method A3. Gradient: 30% to 60% B in |
| | Catalyst: 0.1 eq. Ephos Pd G4 | 10 min. |
| | LCMS: m/z = 766.4 [M + H]⁺ | LCMS: m/z = 666.1 [M + H]⁺. |
| | | ¹H NMR (400 MHz, 6d-DMSO) δ ppm |
| | | 8.67 (s, 1H), 8.14 (s, 1H), 8.05 (s, 2H), |
| | | 7.39-7.25 (m, 1H), 7.15 (t, 1H), 5.37- |
| | | 5.20 (m, 1H), 4.91 (s, 2H), 4.43 ( d, 1H), |
| | | 4.17-4.02 (m, 1H), 3.76-3.63 (m, 1H), |
| | | 3.01-2.71 (m, 3H), 2.60 (s, 3H), 2.50 ( s, |
| | | 3H), 2.21-2.07 (m, 1H), 2.05-1.72 (m, |
| | | 3H), 1.47 ( d, 3H), 1.16 (d, 3H). |
| 120 | tert-butyl ((R)-4-(5-chloro-3-fluoro-6- | (R)-2-amino-4-(5-chloro-3-fluoro-6- |
| | methyl-1-((S)-7-methyl-4-((S)-1-((S)-1- | methyl-1-((S)-7-methyl-4-((S)-1-((S)-1- |
| | methylpyrrolidin-2-yl)ethoxy)-6,7- | methylpyrrolidin-2-yl)ethoxy)-6,7- |
| | dihydro-9H-[1,4]dioxepino[6,5-d] | dihydro-9H-[1,4]dioxepino[6,5-d] |
| | pyrimidin-2-yl)-1H-indazol-4-yl)-3- | pyrimidin-2-yl)-1H-indazol-4-yl)- |
| | cyano-7-fluorobenzo[b]thiophen-2- | 7-fluorobenzo[b]thiophene-3- |
| | yl)carbamate | carbonitrile |
| | 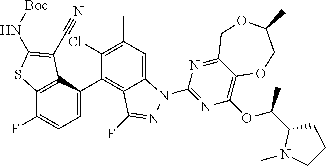 | 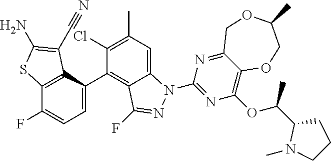 |
| | SM: Intermediate 31 | Method A3. Gradient: 32% to 62% B in |
| | Catalyst: 0.1 eq. Ephos Pd G4 | 10 min. |
| | LCMS: m/z = 766.4 [M + H]⁺ | LCMS: m/z = 666.1 [M + H]⁺ |
| | | ¹H NMR (400 MHz, 6d-DMSO) δ ppm |
| | | 9.97-9.65 (m, 1H), 8.68 (s, 1H), 8.04 (s, |
| | | 2H), 7.36-7.25 (m, 1H), 7.15 (t, 1H), |
| | | 5.39-5.25 (m, 1H), 5.03-4.80 (m, 2H), |
| | | 4.49-4.35 (m, 1H), 4.21-4.06 (m, 1H), |
| | | 3.86-3.74 (m, 1H), 3.16-2.84 (m, 3H), |
| | | 2.61 (s, 3H), 2.50 ( s, 3H), 2.29-1.79 |
| | | (m, 4H), 1.62-1.38 (m, 3H), 1.19 |
| | | (d, 3H) |
| 121 | tert-butyl ((R)-4-(5-chloro-3-fluoro-6- | (R)-2-amino-4-(5-chloro-3-fluoro-6- |
| | methyl-1-((S)-6-methyl-4-((S)-1-((S)-1- | methyl-1-((S)-6-methyl-4-((S)-1-((S)-1- |
| | methylpyrrolidin-2-yl)ethoxy)-6,7- | methylpyrrolidin-2-yl)ethoxy)-6,7- |
| | dihydro-9H-[1,4]dioxepino[6,5-d]- | dihydro-9H-[1,4]dioxepino[6,5-d] |
| | pyrimidin-2-yl)-1H-indazol-4-yl)-3- | pyrimidin-2-yl)-1H-indazol-4-yl)- |
| | cyano-7-fluorobenzo[b]thiophen-2- | 7-fluorobenzo[b]thiophene-3- |
| | yl)carbamate | carbonitrile |
| | 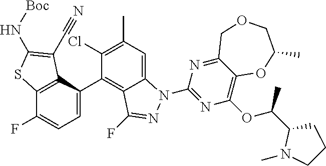 | 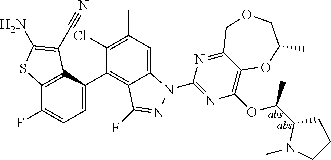 |
| | SM: Intermediate 33 | Method A4. Gradient: 23% to 53% B in |
| | Catalyst: 0.1 eq. Ephos Pd G4 | 15 min |
| | LCMS: m/z = 766.4 [M + H]⁺ | LCMS: m/z = 666.1 [M + H]⁺, |
| | | ¹H NMR (DMSO-d₆, 400 MHz) δ = |
| | | 10.23 (br s, 1H), 8.67 (s, 1H), 8.06 (s, |
| | | 2H), 7.30 (dd, 1H), 7.19-7.11 (m, 1H), |
| | | 5.41-5.30 (m, 1H), 4.98 (d, 1H), 4.81 |
| | | (d, 1H), 4.31-4.21 (m, 1H), 4.09 |
| | | (dd, 1H), 3.90-3.82 (m, 1H), 3.68 (br |
| | | d, 1H), 3.60-3.53 (m, 1H), 3.22-3.13 |
| | | (m, 1H), 3.06 (d, 3H), 2.61 (s, 3H), 2.35- |
| | | 2.24 (m, 1H), 2.15-2.04 (m, 1H), 2.02- |
| | | 1.88 (m, 2H), 1.52 (d, 3H), 1.26 (d, 3H) |
| 122 | tert-butyl ((R)-4-(5-chloro-3-fluoro-6- | (R)-2-amino-4-(5-chloro-3-fluoro-6- |
| | methyl-1-((R)-6-methyl-4-((S)-1-((S)-1- | methyl-1-((R)-6-methyl-4-((S)-1-((S)-1- |
| | methylpyrrolidin-2-yl)ethoxy)-6,7- | methylpyrrolidin-2-yl)ethoxy)-6,7- |
| | dihydro-9H-[1,4]dioxepino[6,5-d] | dihydro-9H-[1,4]dioxepino[6,5-d] |
| | pyrimidin-2-yl)-1H-indazol-4-yl)-3- | pyrimidin-2-yl)-1H-indazol-4-yl)- |
| | cyano-7-fluorobenzo[b]thiophen-2- | fluorobenzo[b]thiophene-3- |
| | yl)carbamate | 7-carbonitrile |
| | 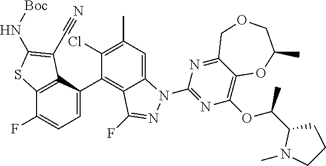 | 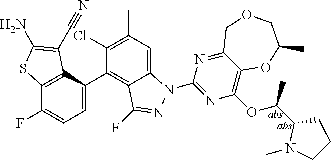 |
| | SM: Intermediate 32 | Method DAICEL CHIRALPAK IK |
| | Catalyst: 0.1 eq. Ephos Pd G4 | (250 mm*25 mm, 10 um); mobile phase: |
| | LCMS: m/z = 766.4 [M + H]⁺ | [CO2-IPA: ACN = 4:1 (0.1% NH3H2O)]; |
| | | B %: 25%, isocratic elution mode |
| | | LCMS: m/z = 666.2 [M + H]⁺. |
| | | 1H NMR (400 MHz, DMSO-d6) δ = |
| | | 8.70 (s, 1H), 8.05 (s, 2H), 7.38-7.23 (m, |
| | | 1H), 7.15 (t, 1H), 5.39-5.16 (m, 1H), |
| | | 5.05-4.86 (m, 1H), 4.86-4.69 (m, 1H), |
| | | 4.24 (t, 1H), 4.06 (d, 1H), 3.76-3.66 (m, |
| | | 1H), 3.19-2.77 (m, 2H), 2.60 (s, 3H), |
| | | 2.52-2.50 (m, 3H), 2.47-2.34 (m, 1H), |
| | | 2.09-1.61 (m, 4H), 1.57-1.34 (m, 3H), |
| | | 1.26 (d, 3H). |

### Example 123

### (R)-2-amino-4-(5-chloro-3-fluoro-6-methyl-1-(1-methyl-8-((S)-1-((S)-1-methylpyrrolidin-2-yl)ethoxy)-2-oxo-1,4-dihydro-2H-pyrimido[5,4-d][1,3]oxazin-6-yl)-1H-indazol -4-yl)-7-fluorobenzo[b]thiophene-3-carbonitrile

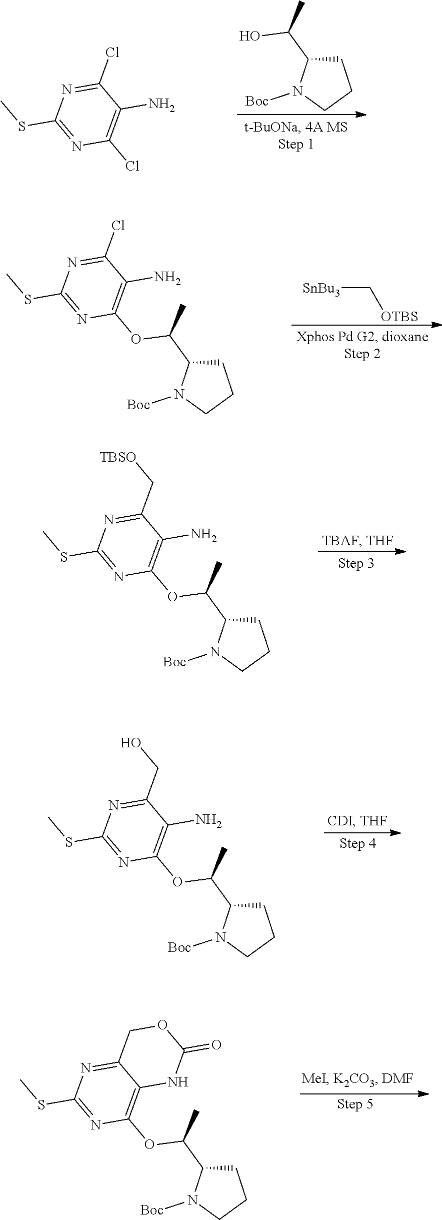

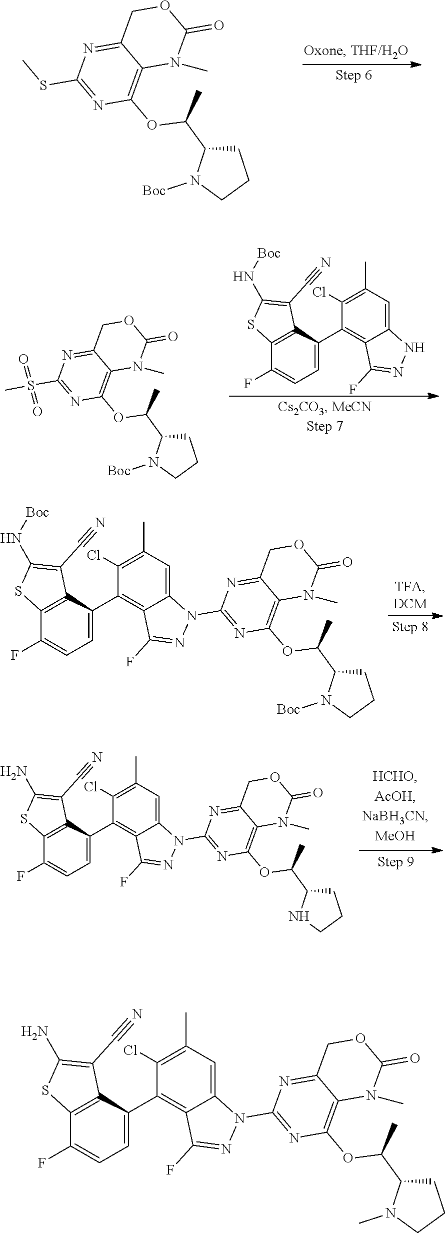

Step 1: Synthesis of tert-butyl (S)-2-((S)-1-((5-amino-6-chloro-2-(methylthio)pyrimidin-4-yl)oxy)ethyl)pyrrolidine-1-carboxylate

To a mixture of 4,6-dichloro-2-(methylthio)pyrimidin-5-amine (950 mg, 4.52 mmol), 4A MS (100 mg) and (S)-1-((S)-1-methylpyrrolidin-2-yl)ethan-1-ol (1.17 g, 5.43 mmol) in toluene (20 mL) was added sodium tert-butoxide (869 mg, 9.04 mmol) and stirred at 0° C. for 0.5 h. The mixture was partitioned with EtOAc (100 mL) and water (50 mL). The organic phase was separated, washed with brine (2×50 mL), dried over Na₂SO₄, filtered and concentrated under reduced pressure. The residue was purified by column chromatography (SiO₂, PE/EtOAc=40/1 to 3/1) to afford the title compound (1.7 g, 96.6% yield) as a yellow gum. LCMS: m/z=389.2 [M+H]⁺

Step 2: Synthesis of tert-butyl (S)-2-((S)-1-((5-amino-6-(((tert-butyldimethylsilyl)oxy)methyl)-2-(methylthio)pyrimidin-4-yl)oxy)ethyl)pyrrolidine-1-carboxylate

A mixture of the title compound from step 1 (1.1 g, 2.83 mmol), tert-butyldimethyl((tributylstannyl)methoxy)silane (3.73 g, 8.49 mmol), and XPHOS-PD-G2 (222 mg, 282 μmol) in dioxane (20 mL) was stirred at 100° C. for 12 h under N₂ atmosphere. The mixture was quenched by the addition of saturated potassium fluoride solution (15 mL) at 0° C. and then diluted with water (10 mL) and extracted with EtOAc (3×50 mL). The combined organic layers were washed with brine (50 mL×2), dried over with Na₂SO₄, filtered and concentrated under reduced pressure. The residue was purified by column chromatography (SiO2, PE/EtOAc 40/1 to 3/1) to give the title compound (900 mg, 39.5% yield) as a yellow gum. LCMS: m/z=499.4 [M+H]⁺

Step 3: Synthesis of tert-butyl (S)-2-((S)-1-((5-amino-6-(hydroxymethyl)-2-(methylthio)pyrimidin-4-yl)oxy)ethyl)pyrrolidine-1-carboxylate

A solution of the title compound from step 2 (900 mg, 1.80 mmol) in THF (10 mL) was treated with TBAF (1 M, 3.61 mL) at 25° C. for 1 h. The mixture was then partitioned between EtOAc (50 mL) and water (30 mL). The organic phase was separated, washed with brine (2×30 mL), dried over Na₂SO₄, filtered and concentrated under reduced pressure. The residue was purified by column chromatography (SiO₂, PE/EtOAc=40/1 to 1/1) to give the title compound (550 mg, 79.2% yield) as a yellow gum. LCMS: m/z=385.2 [M+H]⁺

Step 4: Synthesis of tert-butyl (S)-2-((S)-1-((6-(methylthio)-2-oxo-1,4-dihydro-2H-pyrimido[5,4-d][1,3]oxazin-8-yl)oxy)ethyl)pyrrolidine-1-carboxylate

To a solution of the title compound from Step 3 (500 mg, 1.30 mmol) in THF (10 mL) was added CDI (421 mg, 2.60 mmol) and the mixture was stirred at rt for 12 h. The mixture was diluted with water (50 mL), extracted with EtOAc (100 ml) dried over Na₂SO₄, filtered and concentrated under reduced pressure. The residue was purified by column chromatography (SiO2, PE/(EtOAc: DCM=1:1)=40/1 to 1/1) to give the title compound (390 mg, 73.06% yield) as a yellow solid. LCMS: m/z=411.2 [M+H]⁺

Step 5: Synthesis of tert-butyl (S)-2-((S)-1-((1-methyl-6-(methylthio)-2-oxo-1,4-dihydro-2H-pyrimido[5,4-d][1,3]oxazin-8-yl)oxy)ethyl)pyrrolidine-1-carboxylate

To a mixture of the title compound from step 4 (30 mg, 73.0 μmol) and iodomethane (12.4 mg, 87.7 μmol) in DMF (0.5 mL) was added potassium carbonate (20.2 mg, 146 μmol) at 0° C. under N₂. The mixture was stirred at rt for 12 h then partitioned between EtOAc (10 mL) and water (3 mL). The organic phase was separated, washed with brine (2×5 mL), dried over Na₂SO₄ filtered and concentrated under reduced pressure to give the title compound (25 mg, crude) as a yellow solid. LCMS: m/z=325.2 [M−100+H]⁺

Step 6: Synthesis of tert-butyl (S)-2-((S)-1-((1-methyl-6-(methylsulfonyl)-2-oxo-1,4-dihydro-2H-pyrimido[5,4-d][1,3]oxazin-8-yl)oxy)ethyl)pyrrolidine-1-carboxylate

To a mixture of the title compound from step 5 (25 mg, 58.8 μmol) in THF (0.5 mL) was added a solution of Oxone (108 mg, 176 μmol) in water (0.25 mL) at 0° C., the mixture was stirred at 25° C. for 1 h then quenched by added saturated aqueous sodium thiosulfate (2 ml), diluted with water (2 ml) and extracted with EtOAc (2×5 mL). The combined organic layers were washed with brine (5 mL), dried over Na₂SO₄, filtered and concentrated under reduced pressure to give the title compound (26 mg, crude) as a yellow solid. LCMS: m/z=357.2 [M−99]⁺

Step 7: Synthesis of tert-butyl (S)-2-((S)-1-((6-(4-((R)-2-((tert-butoxycarbonyl)amino)-3-cyano-7-fluorobenzo[b]thiophen-4-yl)-5-chloro-3-fluoro-6-methyl-1H-indazol-1-yl)-1-methyl-2-oxo-1,4-dihydro-2H-pyrimido[5,4-d][1,3]oxazin-8-yl)oxy)ethyl)pyrrolidine-1-carboxylate

A mixture of the title compound from step 6 (26 mg, 56.9 μmol), Intermediate 25 (27.0 mg, 56.9 μmol) and cesium carbonate (55.6 mg, 170 μmol) in ACN (1 mL) was stirred at 60° C. for 1 h. The mixture was partitioned between EtOAc (20 mL) and water (5 mL). The organic phase was separated, washed with brine (5 mL×2), dried over with Na₂SO₄, filtered and concentrated under reduced pressure to give the title compound (45 mg, crude) as a yellow solid. LCMS: m/z=851.4 [M+H]⁺

Step 8: Synthesis of (R)-2-amino-4-(5-chloro-3-fluoro-6-methyl-1-(1-methyl-2-oxo-8-((S)-1-((S)-pyrrolidin-2-yl)ethoxy)-1,4-dihydro-2H-pyrimido[5,4-d][1,3]oxazin-6-yl)-1H-indazol-4-yl)-7-fluorobenzo[b]thiophene-3-carbonitrile

A solution of the title compound from step 7 (35 mg, 41.1 μmol) in DCM (2 mL) was treated with TFA (1.07 g, 9.42 mmol) and the mixture was stirred at rt for 0.5 h. The mixture was concentrated under reduced pressure to give the title compound (31 mg, crude, TFA) as a yellow gum. LCMS: m/z=651.2 [M+H]⁺

Step 9: Synthesis of (R)-2-amino-4-(5-chloro-3-fluoro-6-methyl-1-(1-methyl-8-((S)-1-((S)-1-methylpyrrolidin-2-yl)ethoxy)-2-oxo-1,4-dihydro-2H-pyrimido[5,4-d][1,3]oxazin-6-yl)-1H-indazol-4-yl)-7-fluorobenzo[b]thiophene-3-carbonitrile

To a solution of the title compound (31 mg, 40.5 μmol) and formaldehyde (16.44 mg, 202 μmol, 15.0 μL, 37% aqueous solution) in methanol (2 mL) was added acetic acid (7.30 mg, 121 μmol), the mixture was stirred at 25° C. for 0.5 h. Then sodium cyanoborohydride (12.7 mg, 202 μmol) was added and the resulting mixture was stirred at 25° C. for 0.5 h. The mixture was partitioned between EtOAc (20 mL) and water (5 mL). The organic phase was separated, washed with brine (2×5 mL), dried over with Na₂SO₄, filtered and concentrated under reduced pressure to give a residue. The residue was purified by prep-HPLC (Method A3; gradient:25%-55% B over 10.0 min) and prep-HPLC (Method A4; gradient:23%-53% B over 15.0 min) to give the title compound (19.8 mg, 72.6% yield) as an off-white solid. LCMS: m/z=665.2 [M+H]⁺, ¹H NMR (400 MHz, 6d-DMSO) δ ppm 10.03-9.89 (m, 1H), 8.70 (s, 1H), 8.06 (s, 2H), 7.36-7.25 (m, 1H), 7.16 (t, 1H), 5.63-5.54 (m, 1H), 5.53-5.37 (m, 2H), 4.00-3.85 (m, 1H), 3.42 (s, 3H), 3.24-3.13 (m, 1H), 2.93 (d, 3H), 2.85-2.69 (m, 1H), 2.61 (s, 3H), 2.37-2.23 (m, 1H), 2.14-2.03 (m, 1H), 2.01-1.88 (m, 2H), 1.52 (d, 3H).

### Example 124

### (R)-2-amino-4-(5-chloro-3-fluoro-6-methyl-1-(5-methyl-4-((S)-1-((S)-1-methylpyrrolidin-2-yl)ethoxy)-6-oxo-6,7-dihydro-5H-pyrimido[4,5-b][1,4]oxazin-2-yl)-1H-indazol -4-yl)-7-fluorobenzo[b]thiophene-3-carbonitrile

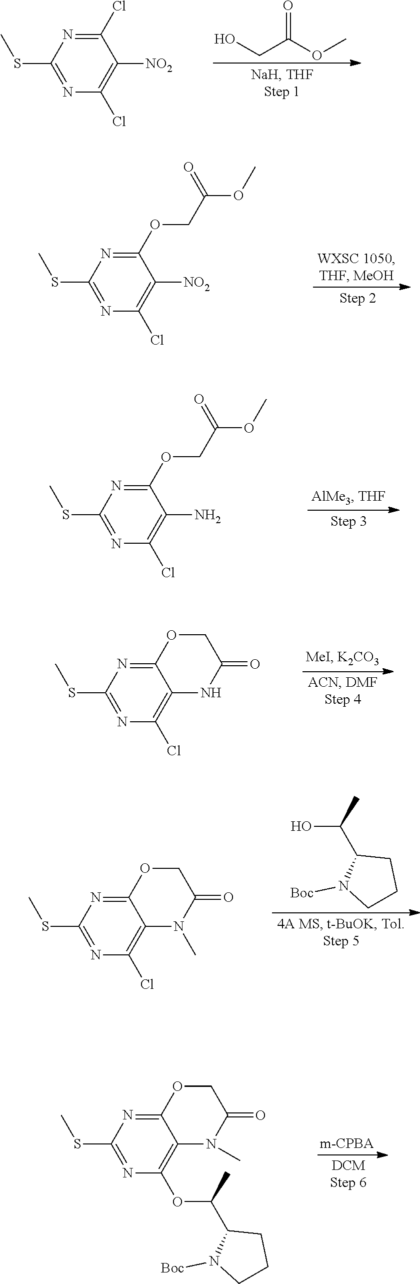

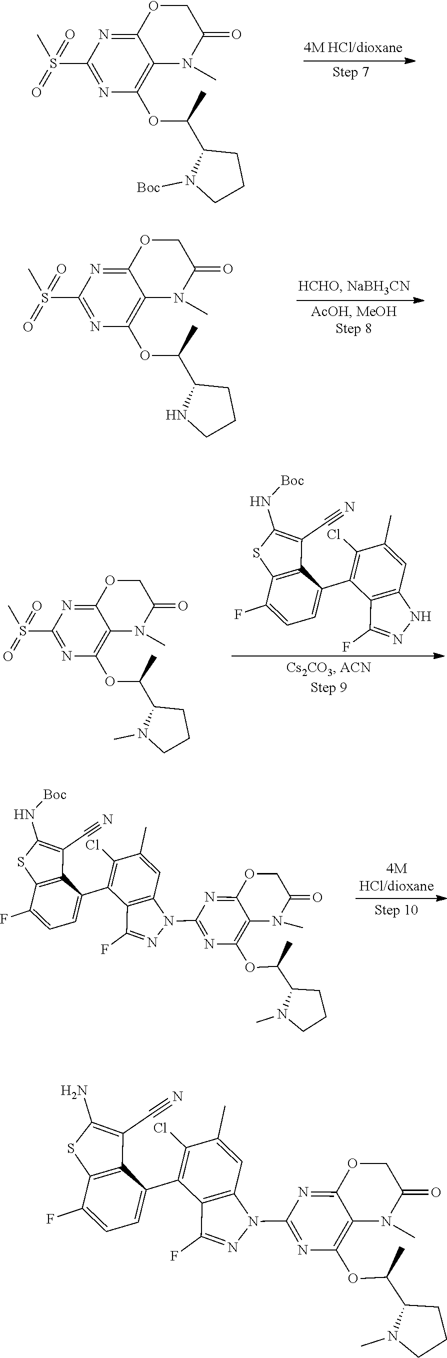

Step 1: Synthesis of methyl 2-((6-chloro-2-(methylthio)-5-nitropyrimidin-4-yl)oxy)acetate

To a solution of methyl 2-hydroxyacetate (2.25 g, 25.0 mmol, 1.93 mL) in THF (100 mL) was added NaH (1.00 g, 25.0 mmol, 60% in oil) at 0° C. under N₂, stirred for 10 min then warmed to rt. To the mixture was added 4,6-dichloro-2-methylsulfanyl-5-nitro-pyrimidine (5.00 g, 20.8 mmol) and stirred at 25° C. for 50 min. The mixture was quenched with sat'd. aqueous NH₄Cl (200 mL), extracted with EtOAc (3×150 mL). The combined organic layers were dried over Na₂SO₄, filtered and concentrated under reduced pressure. The residue was purified by column chromatography SiO₂ (PE:EtOAc=1:0 to 5:1) to give the title compound (4.30 g, 67% yield) as a yellow oil. ¹H NMR (400 MHz, CDCl₃) δ ppm 4.96 (s, 2H), 3.79 (s, 3H), 2.47 (s, 3H).

Step 2: Synthesis of methyl 2-((5-amino-6-chloro-2-(methylthio)pyrimidin-4-yl)oxy)acetate

A solution of the title compound from step 1 (4.00 g, 13.6 mmol) in methanol (40 mL) and tetrahydrofuran (40 mL) was prepared. The fixed bed (named FLR1, volume 5 mL) was completely packed with granular catalyst WXSC1050 (2.7 g). The H2 back pressure regulator was adjusted to 1 Mpa, and the H2 flow rate was {9.000 eq, 13.700 sccm}. The solution was pumped by Pump 1 {S1, P1, 0.4 mL/min} to flow reactor 1 {FLR1, SS, Fixed bed, 6.350(¼″) mm, 5.000 mL, 50.0° C.}. The reaction mixture was continuously collected from the reactor outlet into the container. The mixture was collected with a bottle. On completion, the reaction mixture was filtered and concentrated under reduced pressure to give a residue. The residue was purified by SiO₂ column chromatography (PE:EtOAc=1:0 to 1:1) to give the title compound (2.90 g, 76% yield) as a yellow solid. 1H NMR (400 MHz, CDCl₃) δ ppm 4.96 (s, 2H), 3.79 (s, 3H), 2.47 (s, 3H).

Step 3: Synthesis of 4-chloro-2-(methylthio)-5H-pyrimido[4,5-b][1,4]oxazin-6(7H)-one

To a solution of title compound from step 2 (2.65 g, 10.1 mmol) in THF (53 mL) was added trimethylaluminum (2 M, 15 mL) at 0° C. under N₂, then stirred at 25° C. for 2 h. The mixture was quenched by the addition of sat'd NH₄Cl aqueous solution (150 mL), filtered and the filtrate was extracted with EtOAc (3×100 mL). The combined organic layers were dried over Na₂SO₄, filtered and concentrated under reduced pressure to give the title compound (900 mg, 38% yield) as a white solid. ¹H NMR (400 MHz, DMSO-d6) δ ppm 10.93 (s, 1H), 4.91 (s, 2H), 2.46 (s, 3H).

Step 4: Synthesis of 4-chloro-5-methyl-2-(methylthio)-5H-pyrimido[4,5-b][1,4]oxazin-6(7H)-one

To a mixture of the title compound from step 3 (850 mg, 3.67 mmol) and potassium carbonate (1.27 g, 9.17 mmol) in ACN (17 mL) and DMF (34 mL) was added iodomethane (2.60 g, 18.4 mmol, 1.1 mL), the mixture was stirred at 25° C. for 0.5 h. The reaction mixture was diluted with water (100 mL) and extracted with EtOAc (3×80 mL). The combined organic layers were washed with brine (3×100 mL), dried over Na₂SO₄, filtered and concentrated under reduced pressure to give the title compound (1.18 g, crude) as a yellow solid. ¹H NMR (400 MHz, DMSO-d₆) δ ppm 4.93 (s, 2H), 3.34 (s, 3H), 2.49 (s, 3H).

Step 5: Synthesis of tert-butyl (S)-2-((S)-1-((5-methyl-2-(methylthio)-6-oxo-6,7-dihydro-5H-pyrimido[4,5-b][1,4]oxazin-4-yl)oxy)ethyl)pyrrolidine-1-carboxylate

To a mixture of tert-butyl (2S)-2-[(1S)-1-hydroxyethyl]pyrrolidine-1-carboxylate (1.42 g, 6.59 mmol), 4A molecular sieves (810 mg, 814 μmol) and the title compound from step 4 (810 mg, 3.30 mmol) in toluene (40 mL) was added potassium tert-butoxide (925 mg, 8.24 mmol) under N₂, and stirred at rt for 20 min. The mixture was diluted with EtOAc (50 mL) and sat'd aqueous NH₄Cl (60 mL), extracted with EtOAc (3×50 mL). The combined organic layers were dried over Na₂SO₄, filtered and concentrated under reduced pressure. The residue was purified by SiO₂ column chromatography (PE:EtOAc=1:0 to 2:1) to give the title compound (800 mg, 49% yield) as a yellow oil. LCMS: m/z=425.2 [M+H]⁺

Step 6: Synthesis of tert-butyl (S)-2-((S)-1-((5-methyl-2-(methylsulfonyl)-6-oxo-6,7-dihydro-5H-pyrimido[4,5-b][1,4]oxazin-4-yl)oxy)ethyl)pyrrolidine-1-carboxylate

To a solution of the title compound from step 5 (920 mg, 2.17 mmol) in DCM (16 mL) was added m-CPBA (1.32 g, 6.50 mmol, 85% purity) at 0° C., then stirred at 25° C. for 0.5 h. The mixture was quenched with sat'd Na₂SO₃ aqueous solution (30 mL) and sat'd aqueous NaHCO₃ (30 mL) and extracted with DCM (3×50 mL). The combined organic layers were dried over NaSO₄, filtered and concentrated under reduced pressure. The residue was purified by SiO₂ column chromatography (PE:EtOAc=1:0 to 1:1) to give the title compound (330 mg, 32% yield) as a colorless oil. LCMS: m/z=457 [M+H]⁺

Step 7: Synthesis of 5-methyl-2-(methylsulfonyl)-4-((S)-1-((S)-pyrrolidin-2-yl)ethoxy)-5H-pyrimido[4,5-b][1,4]oxazin-6(7H)-one

A solution of the title compound from step 6 (300 mg, 657 μmol) in HCl/dioxane (4 M, 10 mL) was stirred at 25° C. for 20 minutes. The mixture was then filtered and concentrated under reduced pressure to give the title compound (260 mg, crude, HCl) as a colorless oil. LCMS: m/z=357.1 [M+H]⁺

Step 8: Synthesis of 5-methyl-4-((S)-1-((S)-1-methylpyrrolidin-2-yl)ethoxy)-2-(methylsulfonyl)-5H-pyrimido[4,5-b][1,4]oxazin-6(7H)-one

To a solution of the title compound from step 7 (260 mg, 662 μmol, HCl salt) in MeOH (10 ml) was added formaldehyde (269 mg, 3.31 mmol, 246 μL) and acetic acid (40.0 mg, 662 μmol, 38 μL). The mixture was stirred at 25° C. for 10 min, then sodium cyanoborohydride (208 mg, 3.31 mmol) was added to the mixture, stirred at 25° C. for 20 min. The mixture was then diluted with saturated aqueous sodium bicarbonate (30 mL), concentrated to remove methanol, and extracted with EtOAc (30 mL×3). The combined organic layers were dried over Na₂SO₄, filtered and concentrated under reduced pressure to give the title compound (340 mg, crude) as a yellow oil. LCMS: m/z=371.1 [M+H]⁺

Step 9: Synthesis of tert-butyl ((R)-4-(5-chloro-3-fluoro-6-methyl-1-(5-methyl-4-((S)-1-((S)-1-methylpyrrolidin-2-yl)ethoxy)-6-oxo-6,7-dihydro-5H-pyrimido[4,5-b][1,4]oxazin-2-yl)-1H-indazol-4-yl)-3-cyano-7-fluorobenzo[b]thiophen-2-yl)carbamate

To a mixture of Intermediate 25 (150 mg, 316 μmol) and the title compound from step 8 (315 mg, 850 μmol) in ACN (10 mL) was added Cs₂CO₃ (206 mg, 632 μmol), then the mixture was stirred at 25° C. for 2 h. The mixture was diluted with water (30 mL) and extracted with DCM (3×30 mL). The combined organic layers were dried over Na₂SO₄, filtered and concentrated under reduced pressure. The residue was purified by prep-TLC (silica, EtOAc: MeOH=10:1) to give the title compound (100 mg, 41% yield) as a colorless solid. LCMS: m/z=765.2 [M+H]⁺

Step 10: Synthesis of (R)-2-amino-4-(5-chloro-3-fluoro-6-methyl-1-(5-methyl-4-((S)-1-((S)-1-methylpyrrolidin-2-yl)ethoxy)-6-oxo-6,7-dihydro-5H-pyrimido[4,5-b][1,4]oxazin-2-yl)-1H-indazol-4-yl)-7-fluorobenzo[b]thiophene-3-carbonitrile

A mixture of title compound from step 9 (80.0 mg, 105 μmol) in HCl/dioxane (4 M, 15 mL) was stirred at 35° C. for 2 h. The mixture was then concentrated under reduced pressure and purified by prep-HPLC (Method A3; gradient:25%-55% B over 12.0 min) to give the title compound (23.9 mg, 33% yield) as a white solid. LCMS: m/z=665.1 [M+H]⁺, ¹H NMR (400 MHz, MeOD-d₄) δ ppm 8.72 (s, 1H), 7.27-7.21 (m, 1H), 7.07-7.01 (m, 1H), 5.72-5.62 (m, 3H), 4.91 (s, 2H), 3.49 (s, 3H), 3.40-3.34 (m, 1H), 3.22-3.12 (m, 1H), 2.78-2.60 (m, 7H), 2.23-2.12 (m, 1H), 2.01-1.88 (m, 3H), 1.54 (d, 3H).

### Example 125

### (R)-2-amino-4-(5-chloro-1-(6,8-dimethyl-4-((S)-1-((S)-1-methylpyrrolidin-2-yl)ethoxy)-7-oxo-5,6,7,8-tetrahydropyrimido[4,5-d]pyrimidin-2-yl)-3-fluoro-6-methyl-1H-indazol -4-yl)-7-fluorobenzo[b]thiophene-3-carbonitrile

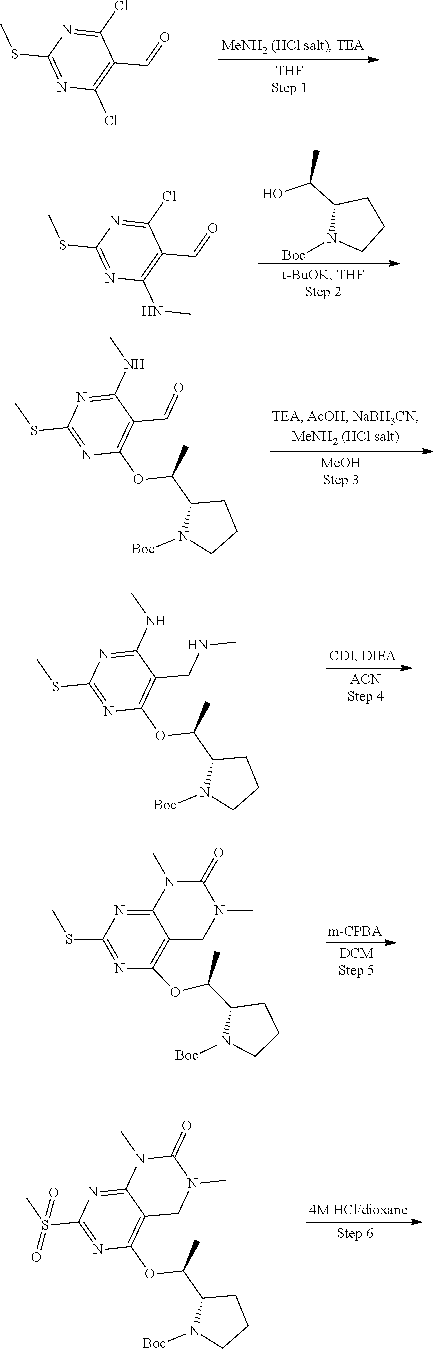

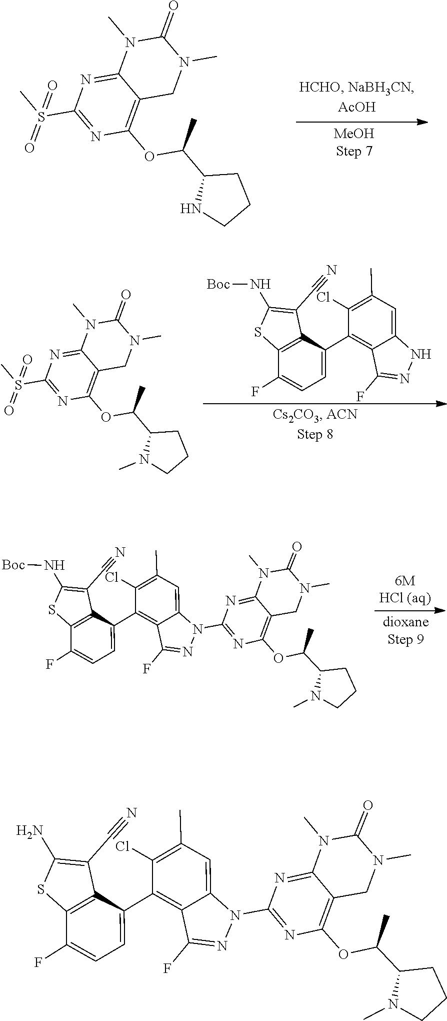

Step 1: Synthesis of 4-chloro-6-(methylamino)-2-(methylthio)pyrimidine-5-carbaldehyde

To a mixture of 4,6-dichloro-2-(methylthio)pyrimidine-5-carbaldehyde (3.00 g, 13.5 mmol) and methylamine hydrochloride (999 mg, 14.8 mmol) in THF (60 mL) was added TEA (3.40 g, 33.6 mmol, 4.7 mL) and stirred at 25° C. for 16 h. The mixture was diluted with water (100 mL) and extracted with EtOAc (3×60 mL). The combined organic layers were dried over Na₂SO₄, filtered and concentrated under reduced pressure to give the title compound (2.54 g, 75% yield) as a yellow solid. ¹H NMR (400 MHz, CDCl₃) δ ppm 10.24 (s, 1H), 9.21 (s, 1H), 3.13 (d, 3H), 2.57 (s, 3H).

Step 2: Synthesis of tert-butyl (S)-2-((S)-1-((5-formyl-6-(methylamino)-2-(methylthio)pyrimidin-4-yl)oxy)ethyl)pyrrolidine-1-carboxylate

To a mixture of the title compound from step 1 (1.00 g, 4.59 mmol) and tert-butyl (2S)-2-[(1S)-1-hydroxyethyl]pyrrolidine-1-carboxylate (1.19 g, 5.51 mmol) in tetrahydrofuran (50 mL) was added potassium tert-butoxide (1.03 g, 9.19 mmol), and stirred at 25° C. for 16 h. The mixture was diluted with water (50 mL) and extracted with EtOAc (3×50 mL). The combined organic layers were dried over Na₂SO₄, filtered and concentrated under reduced pressure. The residue was purified by silica gel column chromatography (PE:EtOAc=1:0 to 3:1) to afford the title compound (1.40 g, 74% yield) as a yellow oil. ¹H NMR (400 MHz, CDCl₃) δ ppm 10.09 (s, 1H), 9.17-9.06 (m, 1H), 5.85-5.48 (m, 1H), 4.13-4.07 (m, 1H), 3.70-3.41 (m, 1H), 3.33-3.22 (m, 1H), 3.08 (d, 3H), 2.53 (s, 3H), 2.02-1.82 (m, 4H), 1.54-1.32 (m, 12H).

Step 3: Synthesis of tert-butyl (S)-2-((S)-1-((6-(methylamino)-5-((methylamino)methyl)-2-(methylthio)pyrimidin-4-yl)oxy)ethyl)pyrrolidine-1-carboxylate

A mixture of the title compound from step 2 (1.40 g, 3.53 mmol), methanamine; hydrochloride (2.38 g, 35.3 mmol) and triethylamine (727 mg, 7.18 mmol, 1 mL) in methanol (14 mL) was stirred at 25° C. for 2.5 h, then acetic acid (1.05 g, 17.5 mmol, 1 mL) and sodium cyanoborohydride (666 mg, 10.6 mmol) were added to the mixture, stirred at 25° C. for 0.5 h. The mixture was diluted with sat'd aqueous NaHCO₃ (30 mL), concentrated to remove methanol, and extracted with EtOAc (3×20 mL). The combined organic layers were dried over Na₂SO₄, filtered and concentrated under reduced pressure to give the title compound (1.45 g, 91% yield) as a yellow oil. ¹H NMR (400 MHz, CDCl₃) δ ppm 6.11-6.00 (m, 1H), 5.44-5.34 (m, 1H), 3.97-3.90 (m, 1H), 3.89-3.80 (m, 2H), 3.43-3.26 (m, 2H), 3.02 (d, 3H), 2.83 (s, 3H), 2.50 (s, 3H), 1.99-1.88 (m, 3H), 1.79-1.75 (m, 1H), 1.31 (d, 3H), 1.26 (s, 9H)

Step 4: Synthesis of tert-butyl (S)-2-((S)-1-((6,8-dimethyl-2-(methylthio)-7-oxo-5,6,7,8-tetrahydropyrimido[4,5-d]pyrimidin-4-yl)oxy)ethyl)pyrrolidine-1-carboxylate

To a mixture of the title compound from step 3 (1.45 g, 3.52 mmol) and CDI (1.14 g, 7.05 mmol) in acetonitrile (20 mL) was added DIEA (1.14 g, 8.81 mmol, 1.53 mL), the mixture was stirred at 80° C. for 16 h. The mixture was quenched by the addition of water (30 mL) and extracted with EtOAc (3×30 mL). The combined organic layers were dried over Na₂SO₄, filtered and concentrated under reduced pressure. The residue was purified by silica gel column chromatography (PE:EtOAc=1:0 to 2:1) to afford the title compound (940 mg, 57% yield) as a white solid. ¹H NMR (400 MHz, CDCl₃) δ ppm 5.81-5.37 (m, 1H), 4.38-4.16 (m, 2H), 4.10-4.06 (m, 1H), 3.68-3.15 (m, 5H), 3.03 (s, 3H), 2.52 (s, 3H), 2.00-1.73 (m, 4H), 1.64 (s, 3H), 1.53-1.34 (m, 9H).

Step 5: Synthesis of tert-butyl (S)-2-((S)-1-((6,8-dimethyl-2-(methylsulfonyl)-7-oxo-5,6,7,8-tetrahydropyrimido[4,5-d]pyrimidin-4-yl)oxy)ethyl)pyrrolidine-1-carboxylate

To a solution of the title compound from step 4 (800 mg, 1.83 mmol) in DCM (10 mL) was added m-CPBA (1.11 g, 5.49 mmol, 85% purity) at 0° C., the mixture was stirred at 25° C. for 1 h. The mixture was quenched by sat'd aqueous Na₂SO₃ (50 mL) and sat'd. aqueous NaHCO₃ (20 mL) and extracted with DCM (3×40 mL). The combined organic layers were dried over Na₂SO₄, filtered and concentrated under reduced pressure. The residue was purified by silica gel column chromatography (PE:EtOAc=1:0 to 1:1) to afford the title compound (730 mg, 82% yield) as white solid. LCMS: m/z=470.2 [M+H]⁺,

Step 6: Synthesis of 1,3-dimethyl-7-(methylsulfonyl)-5-((S)-1-((S)-pyrrolidin-2-yl)ethoxy)-3,4-dihydropyrimido[4,5-d]pyrimidin-2(1H)-one

A mixture of the title compound from step 5 (700 mg, 1.49 mmol) in HCl/dioxane (4 M, 8 mL) was stirred at 25° C. for 0.5 h then concentrated under reduced pressure to give the title compound (605 mg, 100% yield, HCl) as a pink oil. LCMS: m/z=370.1 [M+H]⁺,

Step 7: Synthesis of 1,3-dimethyl-5-((S)-1-((S)-1-methylpyrrolidin-2-yl)ethoxy)-7-(methylsulfonyl)-3,4-dihydropyrimido[4,5-d]pyrimidin-2(1H)-one

A mixture of the title compound from step 6 (605 mg, 1.49 mmol, HCl), formaldehyde (605 mg, 7.45 mmol, 37% aqueous solution) and acetic acid (210 mg, 3.49 mmol, 0.2 mL) in MeOH (10 mL) was stirred at 25° C. for 15 min, then sodium cyanoborohydride (281 mg, 4.47 mmol) was added to the mixture, and stirred at 25° C. for 15 min. The mixture was diluted with sat'd aqueous NaHCO₃ (50 mL) and concentrated to remove MeOH and extracted with EtOAc (3×30 mL). The combined organic layers were dried over Na₂SO₄, filtered and concentrated under reduced pressure. The residue was purified by silica gel column chromatography (PE:EtOAc=1:0 to EtOAc:MeOH=10:1) to afford the title compound (500 mg, 87% yield) as a colorless oil. ¹H NMR (400 MHz, CDCl₃) δ ppm 5.44-5.33 (m, 1H), 4.40-4.29 (m, 2H), 3.41 (s, 3H), 3.30 (s, 3H), 3.12-3.04 (m, 4H), 2.67-2.60 (m, 1H), 2.44 (s, 3H), 2.36-2.27 (m, 1H), 1.91-1.80 (m, 4H), 1.34 (d, 3H).

Step 8: Synthesis of tert-butyl ((R)-4-(5-chloro-1-(6,8-dimethyl-4-((S)-1-((S)-1-methylpyrrolidin-2-yl)ethoxy)-7-oxo-5,6,7,8-tetrahydropyrimido[4,5-d]pyrimidin-2-yl)-3-fluoro-6-methyl-1H-indazol-4-yl)-3-cyano-7-fluorobenzo[b]thiophen-2-yl)carbamate

To a solution of Intermediate 25 (60.0 mg, 126 μmol) and the title compound from step 7 (60.0 mg, 156 μmol) in ACN (2 mL) was added Cs₂CO₃ (82.0 mg, 253 μmol), the mixture was stirred at 25° C. for 1 h. The mixture was diluted with water (20 mL) and extracted with EtOAc (3×20 mL). The combined organic layers were dried over Na₂SO₄, filtered and concentrated under reduced pressure. The residue was purified by silica gel column chromatography (PE:EtOAc=1:0 to EtOAc:MeOH=10:1). To afford the title compound (80.0 mg, 72% yield) as a white solid. LCMS: m/z=778.2 [M+H]⁺,

Step 9: Synthesis of (R)-2-amino-4-(5-chloro-1-(6,8-dimethyl-4-((S)-1-((S)-1-methylpyrrolidin-2-yl)ethoxy)-7-oxo-5,6,7,8-tetrahydropyrimido[4,5-d]pyrimidin-2-yl)-3-fluoro-6-methyl-1H-indazol-4-yl)-7-fluorobenzo[b]thiophene-3-carbonitrile

To a solution of the title compound from step 8 (75.0 mg, 96.4 μmol) in dioxane (5 mL) was added HCl (6 M, 5 mL), the mixture was stirred at 50° C. for 20 minutes. The mixture was basified by addition of aqueous sat'd NaHCO₃ to pH=7-8 and extracted with EtOAc (3×30 mL). The combined organic layers were dried over Na₂SO₄, filtered and concentrated under reduced pressure. The residue was purified by prep-HPLC (column: Phenomenex Luna C18 150*25 mm*10 um; mobile phase: [H2O (0.225% FA)-ACN]; gradient:28%-58% B over 15.0 min). to afford the title compound (54.8 mg, 83% yield) as a yellow solid. LCMS: m/z=678.2 [M+H]⁺, ¹H NMR (400 MHz, CD₃OD) δ ppm 8.73 (s, 1H), 7.27-7.22 (m, 1H), 7.07-7.01 (m, 1H), 5.57-5.48 (m, 1H), 4.45-4.33 (m, 2H), 3.52 (s, 3H), 3.29-3.23 (m, 1H), 3.09-2.98 (m, 4H), 2.71-2.61 (m, 7H), 2.21-2.10 (m, 1H), 1.98-1.80 (m, 3H), 1.46 (d, 3H).

### Example 126

### (R)-2-amino-4-(5-chloro-3-fluoro-6-methyl-1-((R)-9-methyl-4-((S)-1-((S)-1-methylpyrrolidin-2-yl)ethoxy)-6,7-dihydro-9H-[1,4]dioxepino[6,5-d]pyrimidin-2-yl)-1H-indazol-4-yl)-7-fluorobenzo[b]thiophene-3-carbonitrile or (R)-2-amino-4-(5-chloro-3-fluoro-6-methyl-1-((S)-9-methyl-4-((S)-1-((S)-1-methylpyrrolidin-2-yl)ethoxy)-6,7-dihydro-9H-[1,4]dioxepino[6,5-d]pyrimidin-2-yl)-1H-indazol-4-yl)-7-fluorobenzo[b]thiophene-3-carbonitrile

### And Example 127

### (R)-2-amino-4-(5-chloro-3-fluoro-6-methyl-1-((S)-9-methyl-4-((S)-1-((S)-1-methylpyrrolidin-2-yl)ethoxy)-6,7-dihydro-9H-[1,4]dioxepino[6,5-d]pyrimidin-2-yl)-1H-indazol-4-yl)-7-fluorobenzo[b]thiophene-3-carbonitrile or (R)-2-amino-4-(5-chloro-3-fluoro-6-methyl-1-((R)-9-methyl-4-((S)-1-((S)-1-methylpyrrolidin-2-yl)ethoxy)-6,7-dihydro-9H-[1,4]dioxepino[6,5-d]pyrimidin-2-yl)-1H-indazol-4-yl)-7-fluorobenzo[b]thiophene-3-carbonitrile

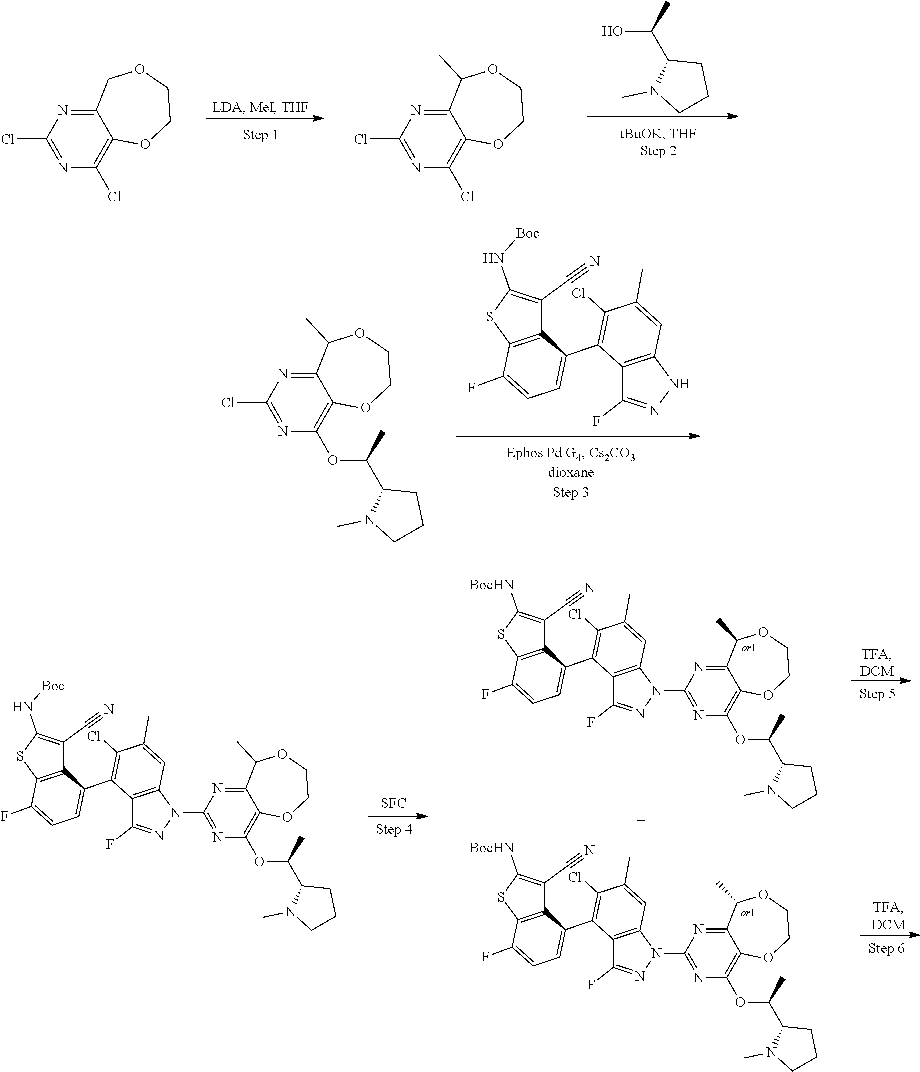

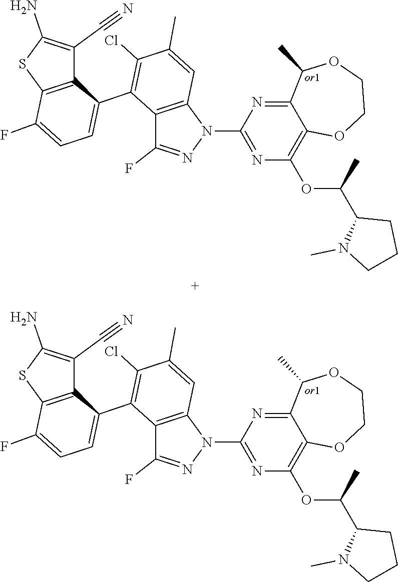

Step 1: Synthesis of 2,4-Dichloro-9-methyl-6,7-dihydro-9H-[1,4]dioxepino[6,5-d]pyrimidine

To a solution of the title compound from Intermediate 26, step 3 (0.5 g, 2.26 mmol) in THF (10 mL) was added LDA in tetrahydrofuran (2 M, 1.24 mL) dropwise at −78° C. under N₂. The mixture was stirred at −78° C. for 0.2 h, followed by the addition of methyl iodide (417 mg, 2.94 mmol). The mixture was stirred at −78° C. for 0.3 h then 25° C. for 0.5 h under N₂. The mixture was quenched by addition of sat'd. NH₄Cl aq. solution (3 mL) at 0° C. and extracted with EtOAc (100 mL). The organic phase was washed with brine (2×50 mL), dried over Na₂SO₄, filtered and concentrated in vacuo. The residue was purified by column chromatography (silica gel, n-hexanes: EtOAc, gradient from 0 to 8% EtOAc) to give the title compound (70 mg, 13% yield) as a white solid. LCMS: m/z=234.9 [M+H]⁺

Step 2: Synthesis of 2-Chloro-9-methyl-4-((S)-1-((S)-1-methylpyrrolidin-2-yl)ethoxy)-6,7-dihydro-9H-[1,4]dioxepino[6,5-d]pyrimidine

To a solution of the title compound from step 1 (50.0 mg, 0.387 mmol) in THF (2 mL) was added potassium tert-butoxide in THF (1 M, 0.33 mL) at 0° C. under N₂. The mixture was stirred at 0° C. for 0.2 h, followed by addition of (S)-1-((S)-1-methylpyrrolidin-2-yl)ethan-1-ol (70 mg, 0.298 mmol). The mixture was stirred at 25° C. for 0.3 h. The mixture was neutralized with formic acid and filtered. The filtrate was purified by reversed-phase HPLC (column: Spherical C18, 40-60 μm, 120 A; mobile phase: [water (0.1% ammonium bicarbonate)-acetonitrile]; gradient:40%-60% acetonitrile) to give the title compound (91 mg, 93% yield) as a gray gel. LCMS: m/z=328.0 [M+H]⁺

Step 3: Synthesis of tert-Butyl ((4R)-4-(5-chloro-3-fluoro-6-methyl-1-(9-methyl-4-((S)-1-((S)-1-methylpyrrolidin-2-yl)ethoxy)-6,7-dihydro-9H-[1,4]dioxepino[6,5-d]pyrimidin-2-yl)-1H-indazol-4-yl)-3-cyano-7-fluorobenzo[b]thiophen-2-yl)carbamate

A mixture of the title compound from step 2 (85.0 mg, 0.259 mmol), Intermediate 25 (135 mg, 0.285 mmol), EPhos Pd G4 (23.8 mg, 25.9 μmol), and Cs₂CO₃ (169 mg, 0.519 mmol) in dioxane (2 mL) was stirred at 100° C. for 1 h under N₂. The mixture was concentrated in vacuo. The residue was purified by column chromatography (silica gel, n-hexanes: EtOAc, gradient from 60% to 100% EtOAc) and further purified by reversed-phase HPLC (column: Spherical C18, 40-60 μm, 120 A; mobile phase: [water (0.1% formic acid)-acetonitrile]; gradient:50%-60% acetonitrile) to give the title compound (87 mg, 44% yield) as a yellow solid. LCMS: m/z=766.4 [M+H]⁺

Step 4: Synthesis of tert-Butyl ((R)-4-(5-chloro-3-fluoro-6-methyl-1-((R)-9-methyl-4-((S)-1-((S)-1-methylpyrrolidin-2-yl)ethoxy)-6,7-dihydro-9H-[1,4]dioxepino[6,5-d]pyrimidin-2-yl)-1H-indazol-4-yl)-3-cyano-7-fluorobenzo[b]thiophen-2-yl)carbamate and tert-Butyl ((R)-4-(5-chloro-3-fluoro-6-methyl-1-((S)-9-methyl-4-((S)-1-((S)-1-methylpyrrolidin-2-yl)ethoxy)-6,7-dihydro-9H-[1,4]dioxepino[6,5-d]pyrimidin-2-yl)-1H-indazol-4-yl)-3-cyano-7-fluorobenzo[b]thiophen-2-yl)carbamate

A solution of the title compound from step 3 (80.0 mg, 0.104 mmol) in methanol (20 mL) was separated by SFC (column: DAICEL CHIRALPAK IK (250 mm×25 mm, 10 um); mobile phase: [carbon dioxide-isopropyl alcohol (0.1% ammonium hydroxide)]; B %: 42%, isocratic elution mode) to give Peak1 tert-Butyl ((R)-4-(5-chloro-3-fluoro-6-methyl-1-((R)-9-methyl-4-((S)-1-((S)-1-methylpyrrolidin-2-yl)ethoxy)-6,7-dihydro-9H-[1,4]dioxepino[6,5-d]pyrimidin-2-yl)-1H-indazol-4-yl)-3-cyano-7-fluorobenzo[b]thiophen-2-yl)carbamate or tert-Butyl ((R)-4-(5-chloro-3-fluoro-6-methyl-1-((S)-9-methyl-4-((S)-1-((S)-1-methylpyrrolidin-2-yl)ethoxy)-6,7-dihydro-9H-[1,4]dioxepino[6,5-d]pyrimidin-2-yl)-1H-indazol-4-yl)-3-cyano-7-fluorobenzo[b]thiophen-2-yl)carbamate (40.0 mg, 50% yield, RT=1.316 min) as a pale-yellow solid and Peak 2 tert-Butyl ((R)-4-(5-chloro-3-fluoro-6-methyl-1-((S)-9-methyl-4-((S)-1-((S)-1-methylpyrrolidin-2-yl)ethoxy)-6,7-dihydro-9H-[1,4]dioxepino[6,5-d]pyrimidin-2-yl)-1H-indazol-4-yl)-3-cyano-7-fluorobenzo[b]thiophen-2-yl)carbamate or tert-Butyl ((R)-4-(5-chloro-3-fluoro-6-methyl-1-((R)-9-methyl-4-((S)-1-((S)-1-methylpyrrolidin-2-yl)ethoxy)-6,7-dihydro-9H-[1,4]dioxepino[6,5-d]pyrimidin-2-yl)-1H-indazol-4-yl)-3-cyano-7-fluorobenzo[b]thiophen-2-yl)carbamate (32.0 mg, 40% yield, RT=1.505 min) as a pale-yellow solid.

Step 5: Synthesis of (R)-2-Amino-4-(5-chloro-3-fluoro-6-methyl-1-((R)-9-methyl-4-((S)-1-((S)-1-methylpyrrolidin-2-yl)ethoxy)-6,7-dihydro-9H-[1,4]dioxepino[6,5-d]pyrimidin-2-yl)-1H-indazol-4-yl)-7-fluorobenzo[b]thiophene-3-carbonitrile or (R)-2-amino-4-(5-chloro-3-fluoro-6-methyl-1-((S)-9-methyl-4-((S)-1-((S)-1-methylpyrrolidin-2-yl)ethoxy)-6,7-dihydro-9H-[1,4]dioxepino[6,5-d]pyrimidin-2-yl)-1H-indazol-4-yl)-7-fluorobenzo[b]thiophene-3-carbonitrile

To a solution of Peak 1 from Step 4 (38 mg, 49.6 μmol) in DCM (1 mL) was added TFA (0.4 mL). The mixture was stirred at 25° C. for 3 h then concentrated in vacuo, the residue was purified by prep-HPLC (column: Phenomenex luna C18 150×25 mm×10 um; mobile phase: [water (0.225% formic acid)-acetonitrile]; gradient:25%-55% B over 15.0 min) to give the titled compound, Example 126, (26.0 mg, 78% yield) as an off-white solid. LCMS: m/z=666.2 [M+H]⁺, ¹H NMR (400 MHz, DMSO-6d) δ=8.66 (s, 1H), 8.07 (s, 2H), 7.32 (dd, 1H), 7.15 (t, 1H), 5.40-5.29 (m, 1H), 4.99 (q, 1H), 4.41-4.30 (m, 1H), 4.19-4.11 (m, 1H), 4.04-3.89 (m, 2H), 3.34-3.31 (m, 3H), 3.21-3.06 (m, 1H), 2.68-2.55 (m, 5H), 2.09-1.92 (m, 1H), 1.89-1.72 (m, 3H), 1.68 (d, 3H), 1.42 (d, 3H).

Step 6: Synthesis of (R)-2-amino-4-(5-chloro-3-fluoro-6-methyl-1-((S)-9-methyl-4-((S)-1-((S)-1-methylpyrrolidin-2-yl)ethoxy)-6,7-dihydro-9H-[1,4]dioxepino[6,5-d]pyrimidin-2-yl)-1H-indazol-4-yl)-7-fluorobenzo[b]thiophene-3-carbonitrile or (R)-2-Amino-4-(5-chloro-3-fluoro-6-methyl-1-((R)-9-methyl-4-((S)-1-((S)-1-methylpyrrolidin-2-yl)ethoxy)-6,7-dihydro-9H-[1,4]dioxepino[6,5-d]pyrimidin-2-yl)-1H-indazol-4-yl)-7-fluorobenzo[b]thiophene-3-carbonitrile

To a solution of peak 2 from step 4 (30 mg, 49.2 μmol) in DCM (1 mL) was added TFA (0.4 mL). The mixture was stirred at 25° C. for 3 h then concentrated in vacuo. The residue was purified by reverse-phase prep-HPLC (column: Phenomenex luna C18 150×25 mm×10 um; mobile phase: [water (0.225% formic acid)-acetonitrile]; gradient:28%-58% B over 15.0 min) to give the title compound, Example 127, (16.9 mg, 65% yield) as an off-white solid. LCMS: m/z=666.2 [M+H]^{+ 1}H NMR (400 MHz, DMSO-6d) δ=8.66 (s, 1H), 8.13 (s, 1H), 8.08 (s, 2H), 7.30 (dd, 1H), 7.15 (t, 1H), 5.39-5.26 (m, 1H), 4.96 (q, 1H), 4.40-4.29 (m, 1H), 4.19-4.10 (m, 1H), 4.07-3.97 (m, 1H), 3.95-3.85 (m, 1H), 3.34-3.14 (m, 4H), 2.81-2.65 (m, 2H), 2.57 (s, 3H), 2.13-1.96 (m, 1H), 1.94-1.73 (m, 3H), 1.68 (d, 3H), 1.43 (d, 3H).

### Example 128

### (R)-2-amino-4-(5-chloro-1-(4-((S)-1-((2S,4S)-1,4-dimethylpyrrolidin-2-yl)ethoxy)-5,7-dihydrofuro[3,4-d]pyrimidin-2-yl)-3-fluoro-6-methyl-1H-indazol-4-yl)-7-fluorobenzo[b]thiophene-3-carbonitrile or (S)-2-amino-4-(5-chloro-1-(4-((S)-1-((2S,4S)-1,4-dimethylpyrrolidin-2-yl)ethoxy)-5,7-dihydrofuro[3,4-d]pyrimidin-2-yl)-3-fluoro-6-methyl-1H-indazol-4-yl)-7-fluorobenzo[b]thiophene-3-carbonitrile

### And Example 129

### (S)-2-amino-4-(5-chloro-1-(4-((S)-1-((2S,4S)-1,4-dimethylpyrrolidin-2-yl)ethoxy)-5,7-dihydrofuro[3,4-d]pyrimidin-2-yl)-3-fluoro-6-methyl-1H-indazol-4-yl)-7-fluorobenzo[b]thiophene-3-carbonitrile or (R)-2-amino-4-(5-chloro-1-(4-((S)-1-((2S,4S)-1,4-dimethylpyrrolidin-2-yl)ethoxy)-5,7-dihydrofuro[3,4-d]pyrimidin-2-yl)-3-fluoro-6-methyl-1H-indazol-4-yl)-7-fluorobenzo[b]thiophene-3-carbonitrile

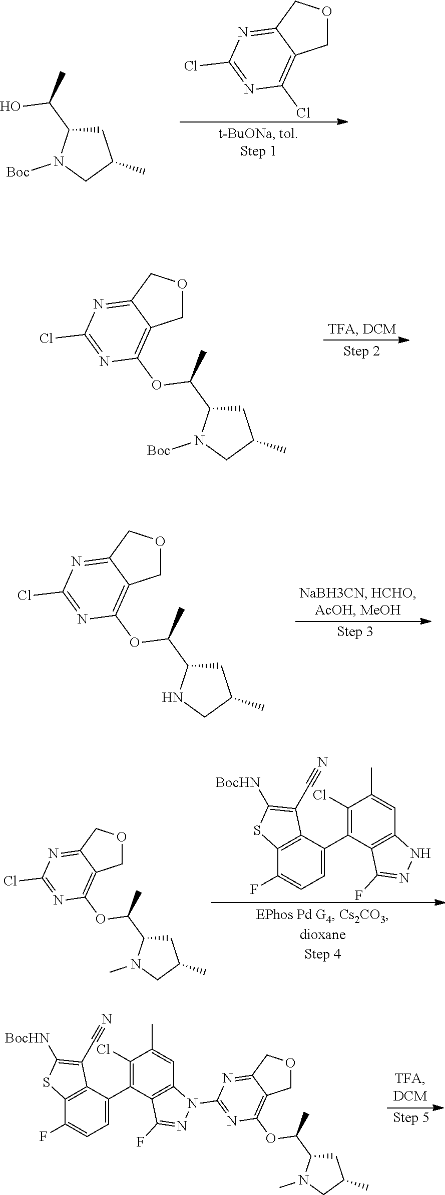

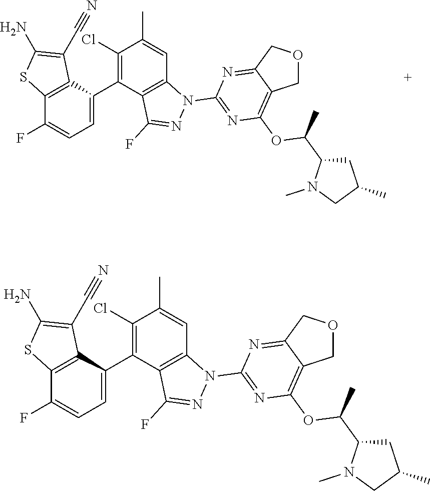

Step 1: Synthesis of tert-butyl (2S,4S)-2-((S)-1-((2-chloro-5,7-dihydrofuro[3,4-d]pyrimidin-4-yl)oxy)ethyl)-4-methylpyrrolidine-1-carboxylate

To a solution of tert-butyl (2S,4S)-2-((S)-1-hydroxyethyl)-4-methylpyrrolidine-1-carboxylate (500 mg, 2.18 mmol), 2,4-dichloro-5,7-dihydrofuro[3,4-d]pyrimidine (416 mg, 2.18 mmol) and molecular sieve (500 mg) in toluene (10 mL) was added sodium tert-butoxide (314.32 mg, 3.27 mmol) at 0° C. The mixture was stirred at 0-25° C. for 12 h. The mixture was diluted with water (30 mL) and extracted with EtOAc (3×30 mL). The combined organic layers were washed with brine, dried over Na₂SO₄, filtered and concentrated under reduced pressure. The crude product was purified by reversed-phase HPLC (0.1% FA condition) to give the title compound (150 mg, 17% yield) as a yellow oil. LCMS: m/z=284.1 [M+H]⁺

Step 2: Synthesis of 2-chloro-4-((S)-1-((2S,4S)-4-methylpyrrolidin-2-yl)ethoxy)-5,7-dihydrofuro[3,4-d]pyrimidine

To a solution of the title compound from step 1 (135 mg, 352 μmol) in DCM (2 mL) was added TFA (1.04 g, 9.09 mmol, 675 μL). The mixture was stirred at 25° C. for 0.5 h then concentrated under reduced pressure to give compound the title compound (116 mg, crude, TFA) as a yellow oil.

Step 3: Synthesis of 2-chloro-4-((S)-1-((2S,4S)-1,4-dimethylpyrrolidin-2-yl)ethoxy)-5,7-dihydrofuro[3,4-d]pyrimidine

To a mixture of the title compound from step 2 (106 mg, 266.48 μmol, TFA salt) and formaldehyde (109 mg, 1.34 mmol, 0.1 mL) in methanol (2 mL) was added acetic acid (52.5 mg, 873 μmol, 0.05 mL). The reaction mixture was stirred at 25° C. for 0.5 h. Then sodium cyanoborohydride (83.7 mg, 1.33 mmol) was added to the mixture and stirred at 25° C. for 0.5 h. The mixture was concentrated under reduced pressure, and the crude product was purified by reversed-phase HPLC (0.1% FA condition) to afford the title compound (90.0 mg, 262 μmol, 98% yield, FA) as a yellow oil. LCMS: m/z=298.1 [M+H]⁺

Step 4: Synthesis of tert-butyl (4-(5-chloro-1-(4-((S)-1-((2S,4S)-1,4-dimethylpyrrolidin-2-yl)ethoxy)-5,7-dihydrofuro[3,4-d]pyrimidin-2-yl)-3-fluoro-6-methyl-1H-indazol-4-yl)-3-cyano-7-fluorobenzo[b]thiophen-2-yl)carbamate

A mixture of the title compound from step 3 (85.0 mg, 247 μmol, FA salt), the title compound from Intermediate 11, step 4 (117 mg, 247 μmol), EPhos Pd G4 (22.7 mg, 24.7 μmol), and Cs₂CO₃ (161 mg, 494 mmol) in dioxane (2 mL) was stirred at 100° C. for 0.5 h under N₂. The reaction mixture was diluted with water (10 mL) and extracted with EtOAc (10 mL×3). The combined organic layers were washed with brine, dried over Na₂SO₄, filtered and concentrated under reduced pressure. The residue was purified by prep-HPLC (Method A4: gradient:38%-68% B over 15.0 min) to give the title compound (120 mg, 141 μmol, 57% yield, TFA) as a white solid. LCMS: m/z=736.4 [M+H]⁺

Step 5: Synthesis of (R)-2-amino-4-(5-chloro-1-(4-((S)-1-((2S,4S)-1,4-dimethylpyrrolidin-2-yl)ethoxy)-5,7-dihydrofuro[3,4-d]pyrimidin-2-yl)-3-fluoro-6-methyl-1H-indazol-4-yl)-7-fluorobenzo[b]thiophene-3-carbonitrile and (S)-2-amino-4-(5-chloro-1-(4-((S)-1-((2S,4S)-1,4-dimethylpyrrolidin-2-yl)ethoxy)-5,7-dihydrofuro[3,4-d]pyrimidin-2-yl)-3-fluoro-6-methyl-1H-indazol-4-yl)-7-fluorobenzo[b]thiophene-3-carbonitrile

To a solution of the title compound from step 4 (110 mg, 149 μmol) in DCM (1.65 mL) was added TFA (844 mg, 7.40 mmol, 550 μL) and the mixture was stirred at 25° C. for 0.5 h then concentrated under reduced pressure. The residue was purified by prep-HPLC (Method A4: gradient:20%-50% B over 15.0 min) and further purified by prep-HPLC (column: Phenomenex luna C18 150*25 mm*10 um; mobile phase: [H2O(0.225% FA)-ACN]; gradient:28%-58% B over 11.0 min) to afford Peak 1, Example 128, (R)-2-amino-4-(5-chloro-1-(4-((S)-1-((2S,4S)-1,4-dimethylpyrrolidin-2-yl)ethoxy)-5,7-dihydrofuro[3,4-d]pyrimidin-2-yl)-3-fluoro-6-methyl-1H-indazol-4-yl)-7-fluorobenzo[b]thiophene-3-carbonitrile or (S)-2-amino-4-(5-chloro-1-(4-((S)-1-((2S,4S)-1,4-dimethylpyrrolidin-2-yl)ethoxy)-5,7-dihydrofuro[3,4-d]pyrimidin-2-yl)-3-fluoro-6-methyl-1H-indazol-4-yl)-7-fluorobenzo[b]thiophene-3-carbonitrile (11.9 mg, 15.8 μmol, 10% yield, TFA) as an off-white solid and Peak 2, Example 129, (S)-2-amino-4-(5-chloro-1-(4-((S)-1-((2S,4S)-1,4-dimethylpyrrolidin-2-yl)ethoxy)-5,7-dihydrofuro[3,4-d]pyrimidin-2-yl)-3-fluoro-6-methyl-1H-indazol-4-yl)-7-fluorobenzo[b]thiophene-3-carbonitrile or (R)-2-amino-4-(5-chloro-1-(4-((S)-1-((2S,4S)-1,4-dimethylpyrrolidin-2-yl)ethoxy)-5,7-dihydrofuro[3,4-d]pyrimidin-2-yl)-3-fluoro-6-methyl-1H-indazol-4-yl)-7-fluorobenzo[b]thiophene-3-carbonitrile (20.3 mg, 27.1 μmol, 18% yield, TFA) as an off-white solid.

Peak 1, Example 128: LCMS: m/z=636.3 [M+H]^{+ 1}H NMR (400 MHz, 6d-DMSO): δ ppm 10.55-10.21 (m, 1H), 8.71 (s, 1H), 8.07 (s, 2H), 7.36-7.26 (m, 1H), 7.23-7.07 (m, 1H), 5.60-5.44 (m, 1H), 5.08 (br d, 4H), 3.33 (s, 3H), 3.16-2.99 (m, 1H), 2.93-2.72 (m, 2H), 2.61 (s, 3H), 2.36-2.25 (m, 1H), 1.68-1.38 (m, 4H), 1.10-1.02 (m, 3H), SFC (Rt=1.015, 100% ee).

Peak 2, Example 129: LCMS: m/z=636.3 [M+H]^{+ 1}H NMR (400 MHz, 6d-DMSO): δ ppm 10.55-10.06 (m, 1H), 8.69 (s, 1H), 8.07 (s, 2H), 7.37-7.27 (m, 1H), 7.16 (t, 1H), 5.58-5.46 (m, 1H), 5.08 (s, 4H), 3.33 (br s, 3H), 3.04-2.66 (m, 3H), 2.61 (s, 3H), 2.31-2.21 (m, 1H), 1.47 (br d, 4H), 1.05 (br d, 3H), SFC (Rt=1.561, 93.7% ee).

### Example 130

### (S)-2-amino-4-(5-chloro-3-fluoro-6-methoxy-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (R)-2-amino-4-(5-chloro-3-fluoro-6-methoxy-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

### And Example 131

### (R)-2-amino-4-(5-chloro-3-fluoro-6-methoxy-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (S)-2-amino-4-(5-chloro-3-fluoro-6-methoxy-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

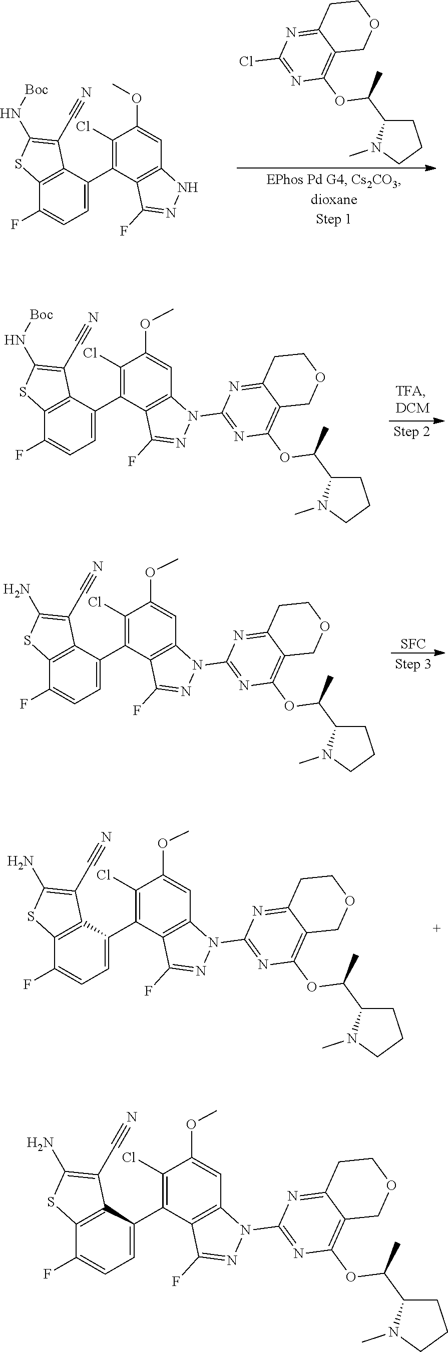

Step 1: Synthesis of tert-butyl (4-(5-chloro-3-fluoro-6-methoxy-1-(4-((S)-1-((S)-1-methylpyrrolidin-2-yl)ethoxy)-7,8-dihydro-5H-pyrano[4,3-d]pyrimidin-2-yl)-1H-indazol-4-yl)-3-cyano-7-fluorobenzo[b]thiophen-2-yl)carbamate

A mixture of Intermediate 35 (2 g, 4.07 mmol), Intermediate 1 (1.46 g, 4.89 mmol), Cs₂CO₃ (2.65 g, 8.15 mmol) and EPhos Pd G4 (374 mg, 0.4 mmol) in dioxane (45 mL) was degassed and purged with N₂ then the mixture was stirred at 100° C. for 0.5 h under N₂. The mixture was diluted with water (200 mL), extracted with EtOAc (100 mL×3). The combined organic layers were washed with brine (200 mL), dried over Na₂SO₄, filtered and concentrated in vacuo to give the title compound (3.2 g, crude) as a yellow oil. LCMS: m/z=752.2 [M+H]⁺

Step 2: Synthesis of 2-amino-4-(5-chloro-3-fluoro-6-methoxy-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

To a solution of the title compound from step 1 (3 g, 3.99 mmol) in DCM (60 mL) was added TFA (30.70 g, 270 mmol, 20 mL) at 20° C. stirred for 1 h. The mixture was concentrated in vacuo. The crude product was purified by prep-HPLC (column: Phenomenex luna C18 (250×70 mm, 10 um); mobile phase: [water (0.225% formic acid)-acetonitrile]; gradient:30%-60% B over 15.0 min) to give the title compound (2.5 g, 96% yield) as a yellow solid. LCMS: m/z=652.2 [M+H]⁺

Step 3: Synthesis of (S)-2-amino-4-(5-chloro-3-fluoro-6-methoxy-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile, and (R)-2-amino-4-(5-chloro-3-fluoro-6-methoxy-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile.

The title compound from step 2 (2.3 g, 3.53 mmol) was separated by chiral SFC ((column: DAICEL CHIRALPAK IG (250 mm×30 mm, 10 um); mobile phase: [carbon dioxide-ethanol: acetonitrile=4:1 (0.1% ammonium hydroxide)]; B %: 45%, isocratic elution mode) to give peak 1 which was further purified by reversed-phase HPLC (column: Phenomenex luna C18 250×50 mm×10 um; mobile phase: [water (0.225% formic acid)-acetonitrile]; gradient:30%-60% B over 22.0 min) to give Example 130, (S)-2-amino-4-(5-chloro-3-fluoro-6-methoxy-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (R)-2-amino-4-(5-chloro-3-fluoro-6-methoxy-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile. (867 mg, 35% yield, SFC (Rt=1.104 minutes, 100% ee) as a yellow solid and peak 2 which was further purified by reversed-phase HPLC (column: Phenomenex luna C18 250×50 mm×10 um; mobile phase: [water (0.225% formic acid)-acetonitrile]; gradient:30%-60% B over 22.0 min) to give Example 131, (R)-2-amino-4-(5-chloro-3-fluoro-6-methoxy-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (S)-2-amino-4-(5-chloro-3-fluoro-6-methoxy-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile. (759.3 mg, 31% yield, SFC Rt=1.771 minutes, 99.1% ee) as a yellow solid

Peak 1, Example 130, LCMS: m/z=652.2 [M+H]⁺. 1H NMR (400 MHz, 6d-DMSO): δ=8.38 (d, 1H), 8.16 (s, 1H), 8.06 (s, 2H), 7.29 (dd, 1H), 7.18-7.11 (m, 1H), 5.45-5.33 (m, 1H), 4.66-4.51 (m, 2H), 4.06 (s, 3H), 4.00 (t, 2H), 3.06-2.97 (m, 1H), 2.91 (t, 2H), 2.76-2.68 (m, 1H), 2.40 (s, 3H), 2.34-2.25 (m, 1H), 1.99-1.84 (m, 1H), 1.77-1.59 (m, 3H), 1.36 (d,)

Peak 2, Example 131, LCMS: m/z=652.2 [M+H]⁺. 1H NMR (400 MHz, 6d-DMSO): δ=8.37 (d, 1H), 8.16 (s, 1H), 8.06 (s, 2H), 7.28 (dd, 1H), 7.19-7.11 (m, 1H), 5.44-5.34 (m, 1H), 4.66-4.50 (m, 2H), 4.06 (s, 3H), 4.00 (t, 2H), 2.99-2.94 (m, 1H), 2.94-2.89 (m, 2H), 2.69-2.60 (m, 1H), 2.37 (s, 3H), 2.29-2.18 (m, 1H), 1.93-1.83 (m, 1H), 1.76-1.58 (m, 3H), 1.35 (d, 3H)

### Example 132

### (R)-2-amino-7-fluoro-4-(3-fluoro-1-(4-((S)-1-((S)-1-methylpyrrolidin-2-yl)ethoxy)-7,8-dihydro-5H-pyrano[4,3-d]pyrimidin-2-yl)-5,7-dihydro-1H-furo[3,4-f]indazol-4-yl)benzo[b]thiophene-3-carbonitrile or (S)-2-amino-7-fluoro-4-(3-fluoro-1-(4-((S)-1-((S)-1-methylpyrrolidin-2-yl)ethoxy)-7,8-dihydro-5H-pyrano[4,3-d]pyrimidin-2-yl)-5,7-dihydro-1H-furo[3,4-f]indazol-4-yl)benzo[b]thiophene-3-carbonitrile

### And Example 133

### (S)-2-amino-7-fluoro-4-(3-fluoro-1-(4-((S)-1-((S)-1-methylpyrrolidin-2-yl)ethoxy)-7,8-dihydro-5H-pyrano[4,3-d]pyrimidin-2-yl)-5,7-dihydro-1H-furo[3,4-f]indazol-4-yl)benzo[b]thiophene-3-carbonitrile or (R)-2-amino-7-fluoro-4-(3-fluoro-1-(4-((S)-1-((S)-1-methylpyrrolidin-2-yl)ethoxy)-7,8-dihydro-5H-pyrano[4,3-d]pyrimidin-2-yl)-5,7-dihydro-1H-furo[3,4-f]indazol-4-yl)benzo[b]thiophene-3-carbonitrile

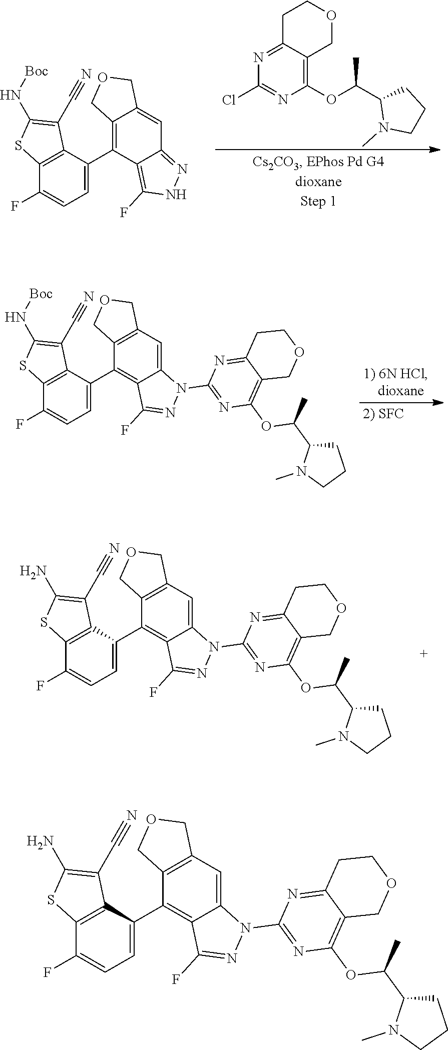

Step 1: Synthesis of tert-butyl (3-cyano-7-fluoro-4-(3-fluoro-1-(4-((S)-1-((S)-1-methylpyrrolidin-2-yl)ethoxy)-7,8-dihydro-5H-pyrano[4,3-d]pyrimidin-2-yl)-5,7-dihydro-1H-furo[3,4-f]indazol-4-yl)benzo[b]thiophen-2-yl)carbamate

A mixture of Intermediate 36 (280 mg, 598 μmol), Intermediate 1 (196 mg, 658 μmol), Cs₂CO₃ (389 mg, 1.20 mmol) and EPhos Pd G4 (55.0 mg, 59.8 μmol) in dioxane (20 mL) was degassed and purged with N₂ then the mixture was stirred at 80° C. for 1 h under N₂. The reaction mixture was filtered and concentrated under reduced pressure. The residue was purified by silica gel column chromatography (PE:EtOAc=1:0 to EtOAc:MeOH=10:1) to afford the title compound (350 mg, 76% yield) as a yellow solid. LCMS: m/z=730.3 [M+H]⁺

Step 2: Synthesis of (R)-2-amino-7-fluoro-4-(3-fluoro-1-(4-((S)-1-((S)-1-methylpyrrolidin-2-yl)ethoxy)-7,8-dihydro-5H-pyrano[4,3-d]pyrimidin-2-yl)-5,7-dihydro-1H-furo[3,4-f]indazol-4-yl)benzo[b]thiophene-3-carbonitrile and (S)-2-amino-7-fluoro-4-(3-fluoro-1-(4-((S)-1-((S)-1-methylpyrrolidin-2-yl)ethoxy)-7,8-dihydro-5H-pyrano[4,3-d]pyrimidin-2-yl)-5,7-dihydro-1H-furo[3,4-f]indazol-4-yl)benzo[b]thiophene-3-carbonitrile

To a solution of the title compound in Step 1 (350 mg, 480 μmol) in dioxane (6 mL) was added HCl (6 M, 6 mL, aq.), the mixture was heated to 50° C. and stirred for 20 min. The mixture was basified by addition of 40 mL of sat'd aqueous NaHCO₃ and extracted with DCM (30 mL×3). The combined organic layers were dried over anhydrous Na₂SO₄, filtered and concentrated under reduced pressure to give a residue. The residue was purified by prep-HPLC (column: Waters Xbridge Prep OBD C18 150*40 mm*10 um; mobile phase: [H2O(0.05% NH3H2O)-ACN]; gradient:62%-92% B over 15.0 min) to give title compound (210 mg) as a white solid, which was further separated by SFC (column: DAICEL CHIRALPAK AD(250 mm*30 mm, 10 um); mobile phase: [CO2-EtOH (0.1% NH3H2O)]; B %: 40%, isocratic elution mode) to afford:

Peak 1, Example 132, (79.8 mg, 38% yield) as a white solid.

### (R)-2-amino-7-fluoro-4-(3-fluoro-1-(4-((S)-1-((S)-1-methylpyrrolidin-2-yl)ethoxy)-7,8-dihydro-5H-pyrano[4,3-d]pyrimidin-2-yl)-5,7-dihydro-1H-furo[3,4-f]indazol-4-yl)benzo[b]thiophene-3-carbonitrile or (S)-2-amino-7-fluoro-4-(3-fluoro-1-(4-((S)-1-((S)-1-methylpyrrolidin-2-yl)ethoxy)-7,8-dihydro-5H-pyrano[4,3-d]pyrimidin-2-yl)-5,7-dihydro-1H-furo[3,4-f]indazol-4-yl)benzo[b]thiophene-3-carbonitrile

Peak 2, Example 133, (71.4 mg, 34% yield) as a white solid.

### (S)-2-amino-7-fluoro-4-(3-fluoro-1-(4-((S)-1-((S)-1-methylpyrrolidin-2-yl)ethoxy)-7,8-dihydro-5H-pyrano[4,3-d]pyrimidin-2-yl)-5,7-dihydro-1H-furo[3,4-f]indazol-4-yl)benzo[b]thiophene-3-carbonitrile or (R)-2-amino-7-fluoro-4-(3-fluoro-1-(4-((S)-1-((S)-1-methylpyrrolidin-2-yl)ethoxy)-7,8-dihydro-5H-pyrano[4,3-d]pyrimidin-2-yl)-5,7-dihydro-1H-furo[3,4-f]indazol-4-yl)benzo[b]thiophene-3-carbonitrile

### Example 134

### (S)-2-amino-4-(5-chloro-3-fluoro-6-(hydroxymethyl)-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (R)-2-amino-4-(5-chloro-3-fluoro-6-(hydroxymethyl)-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

### And Example 135

### (R)-2-amino-4-(5-chloro-3-fluoro-6-(hydroxymethyl)-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (S)-2-amino-4-(5-chloro-3-fluoro-6-(hydroxymethyl)-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

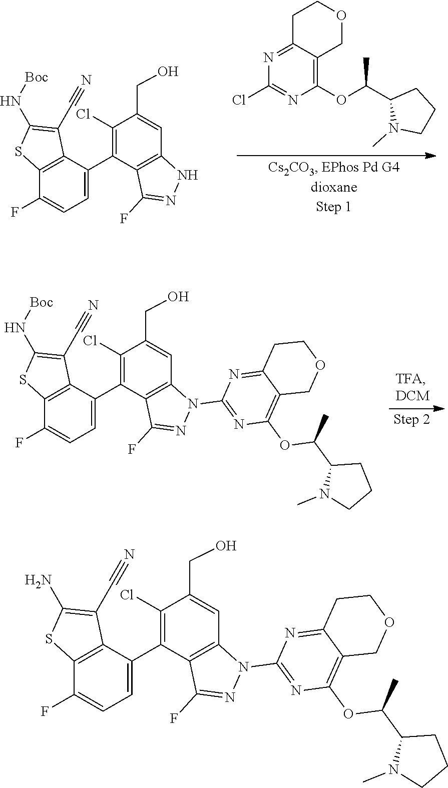 [SMILES: CC(C)(C)OC(=O)Nc1sc2c(F)ccc(-c3c(Cl)c(CO)cc4[nH]nc(F)c34)c2c1C#N ; C[C@H](Oc1nc(Cl)nc2c1COCC2)[C@@H]1CCCN1C ; C[C@H](Oc1nc(-n2nc(F)c3c(-c4ccc(F)c5sc(NC(=O)OC(C)(C)C)c(C#N)c45)c(Cl)c(CO)cc32)nc2c1COCC2)[C@@H]1CCCN1C ; C[C@H](Oc1nc(-n2nc(F)c3c(-c4ccc(F)c5sc(N)c(C#N)c45)c(Cl)c(CO)cc32)nc2c1COCC2)[C@@H]1CCCN1C]

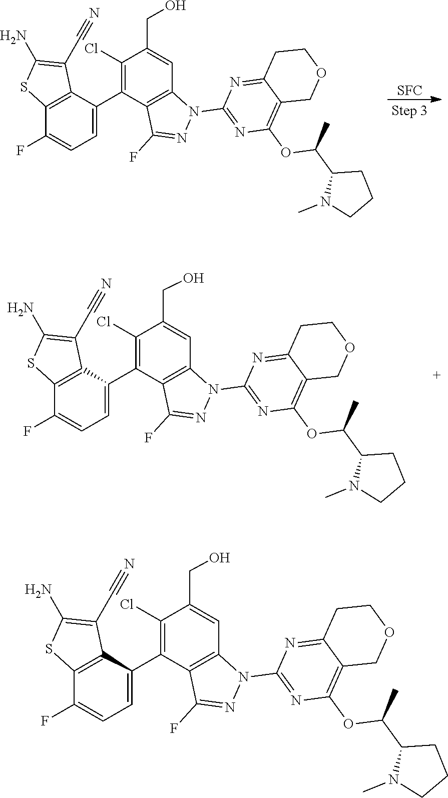

Step 1: Synthesis of tert-butyl (4-(5-chloro-3-fluoro-6-(hydroxymethyl)-1-(4-((S)-1-((S)-1-methylpyrrolidin-2-yl)ethoxy)-7,8-dihydro-5H-pyrano[4,3-d]pyrimidin-2-yl)-1H-indazol-4-yl)-3-cyano-7-fluorobenzo[b]thiophen-2-yl)carbamate

The mixture of Intermediate 38 (1.3 g, 2.65 mmol), Intermediate 1 (946 mg, 3.18 mmol), EPhos Pd G4 (243 mg, 264 μmol), Cs₂CO₃ (1.73 g, 5.30 mmol, 2 eq) in dioxane (10 mL) was degassed and purged with N₂ (3 times) then stirred at 80° C. for 0.5 h under N₂. The reaction mixture was filtered, and filtrate was concentrated in vacuo. The residue was purified by Prep-HPLC (column: Phenomenex luna C18 150*40 mm*15 um; mobile phase: [H2O(0.1% TFA)-ACN]; gradient:40%-70% B over 15.0 min) to give the title compound (1.1 g, 55% yield) as a yellow solid. 1H NMR (DMSO-d6, 400 MHz) δ 8.99 (br s, 1H), 7.0-7.3 (m, 2H), 5.89 (br d, 1H), 5.4-5.5 (m, 1H), 4.75 (br s, 2H), 4.62 (br d, 2H), 4.02 (q, 4H), 2.89 (br d, 2H), 2.6-2.8 (m, 3H), 1.7-2.0 (m, 4H), 1.4-1.5 (m, 12H)

Step 2: Preparation of tert-butyl ((R)-4-(5-chloro-3-fluoro-6-(hydroxymethyl)-1-(4-((S)-1-((S)-1-methylpyrrolidin-2-yl)ethoxy)-7,8-dihydro-5H-pyrano[4,3-d]pyrimidin-2-yl)-1H-indazol-4-yl)-3-cyano-7-fluorobenzo[b]thiophen-2-yl)carbamate and tert-butyl ((S)-4-(5-chloro-3-fluoro-6-(hydroxymethyl)-1-(4-((S)-1-((S)-1-methylpyrrolidin-2-yl)ethoxy)-7,8-dihydro-5H-pyrano[4,3-d]pyrimidin-2-yl)-1H-indazol-4-yl)-3-cyano-7-fluorobenzo[b]thiophen-2-yl)carbamate

The title compound from step 1 was separated by prep-HPLC (column: Phenomenex Luna C18 150*25 mm*10 um; mobile phase: [H2O(0.1% TFA)-ACN]; gradient:25%-55% B over 15.0 min) to give compound peak 1, tert-butyl ((R)-4-(5-chloro-3-fluoro-6-(hydroxymethyl)-1-(4-((S)-1-((S)-1-methylpyrrolidin-2-yl)ethoxy)-7,8-dihydro-5H-pyrano[4,3-d]pyrimidin-2-yl)-1H-indazol-4-yl)-3-cyano-7-fluorobenzo[b]thiophen-2-yl)carbamate or tert-butyl ((S)-4-(5-chloro-3-fluoro-6-(hydroxymethyl)-1-(4-((S)-1-((S)-1-methylpyrrolidin-2-yl)ethoxy)-7,8-dihydro-5H-pyrano[4,3-d]pyrimidin-2-yl)-1H-indazol-4-yl)-3-cyano-7-fluorobenzo[b]thiophen-2-yl)carbamate (30.0 mg, 39.9 μmol, 23% yield) as a white solid, LCMS: m/z=752.2 [M+H]⁺ and peak 2, tert-butyl ((S)-4-(5-chloro-3-fluoro-6-(hydroxymethyl)-1-(4-((S)-1-((S)-1-methylpyrrolidin-2-yl)ethoxy)-7,8-dihydro-5H-pyrano[4,3-d]pyrimidin-2-yl)-1H-indazol-4-yl)-3-cyano-7-fluorobenzo[b]thiophen-2-yl)carbamate or tert-butyl ((R)-4-(5-chloro-3-fluoro-6-(hydroxymethyl)-1-(4-((S)-1-((S)-1-methylpyrrolidin-2-yl)ethoxy)-7,8-dihydro-5H-pyrano[4,3-d]pyrimidin-2-yl)-1H-indazol-4-yl)-3-cyano-7-fluorobenzo[b]thiophen-2-yl)carbamate (30.0 mg, 38.6 μmol, 22% yield) as a white solid, LCMS: m/z=752.2 [M+H]⁺.

Step 3: Synthesis of (R)-2-amino-4-(5-chloro-3-fluoro-6-(hydroxymethyl)-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile and (S)-2-amino-4-(5-chloro-3-fluoro-6-(hydroxymethyl)-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

To a solution of Peak 1 from Step 1 (27.0 mg, 35.9 μmol) in DCM (0.9 mL) was added TFA (414 mg, 3.63 mmol, 270 μL). The mixture was stirred at 25° C. for 0.5 h then concentrated under reduced pressure to give a residue. The residue was purified by prep-HPLC (column: Phenomenex Luna C18 150*25 mm*10 um; mobile phase: [H2O(0.1% TFA)-ACN]; gradient:13%-43% B over 15.0 min) to give Example 134, (R)-2-amino-4-(5-chloro-3-fluoro-6-(hydroxymethyl)-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (S)-2-amino-4-(5-chloro-3-fluoro-6-(hydroxymethyl)-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile (14.8 mg, 19.3 μmol, 53% yield, 100% ee) as a white solid.

To a solution of Peak 2 from step 1 (27.0 mg, 35.9 μmol) in DCM (0.9 mL) was added TFA (414 mg, 3.63 mmol, 270 μL). The mixture was stirred at 25° C. for 0.5 h then concentrated under reduced pressure to give a residue. The residue was purified by prep-HPLC (column: Phenomenex Luna C18 150*25 mm*10 um; mobile phase: [H2O(0.1% TFA)-ACN]; gradient:13%-43% B over 15.0 min) to give Example 135 (12.9 mg, 16.8 μmol, 46% yield, TFA, 99% ee) as a white solid.

Example 134, LCMS: m/z=652.2 [M+H]⁺, ¹H NMR (400 MHz, 6d-DMSO): δ ppm 10.30-10.14 (m, 1H), 9.01 (s, 1H), 8.09 (s, 2H), 7.36-7.27 (m, 1H), 7.17 (t, 1H), 5.96 (br s, 1H), 5.58-5.45 (m, 1H), 4.83-4.73 (m, 2H), 4.73-4.60 (m, 2H), 4.11-3.93 (m, 2H), 3.87-3.74 (m, 1H), 3.60-3.54 (m, 1H), 3.23-3.13 (m, 1H), 2.97 (d, 3H), 2.93 (br t, 2H), 2.35-2.28 (m, 1H), 2.15-2.06 (m, 1H), 2.04-1.86 (m, 2H), 1.54 (d, 3H).

Example 135, LCMS: m/z=652.2 [M+H]⁺, ¹H NMR (400 MHz, 6d-DMSO): δ ppm 9.97-9.80 (m, 1H), 9.03 (s, 1H), 8.07 (s, 2H), 7.37-7.27 (m, 1H), 7.21-7.12 (m, 1H), 6.09-5.87 (m, 1H), 5.54-5.41 (m, 1H), 4.85-4.73 (m, 2H), 4.72-4.60 (m, 2H), 4.09-3.95 (m, 2H), 3.88-3.77 (m, 1H), 3.61-3.54 (m, 1H), 3.25-3.17 (m, 1H), 3.00 (d, 3H), 2.92 (br t, 2H), 2.37-2.30 (m, 1H), 2.18-2.09 (m, 1H), 2.04-1.87 (m, 2H), 1.53 (d, 3H).

### Example 136

### (S)-2-amino-4-(5-chloro-3-fluoro-6-(fluoromethyl)-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (R)-2-amino-4-(5-chloro-3-fluoro-6-(fluoromethyl)-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

### And Example 137

### (R)-2-amino-4-(5-chloro-3-fluoro-6-(fluoromethyl)-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (S)-2-amino-4-(5-chloro-3-fluoro-6-(fluoromethyl)-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

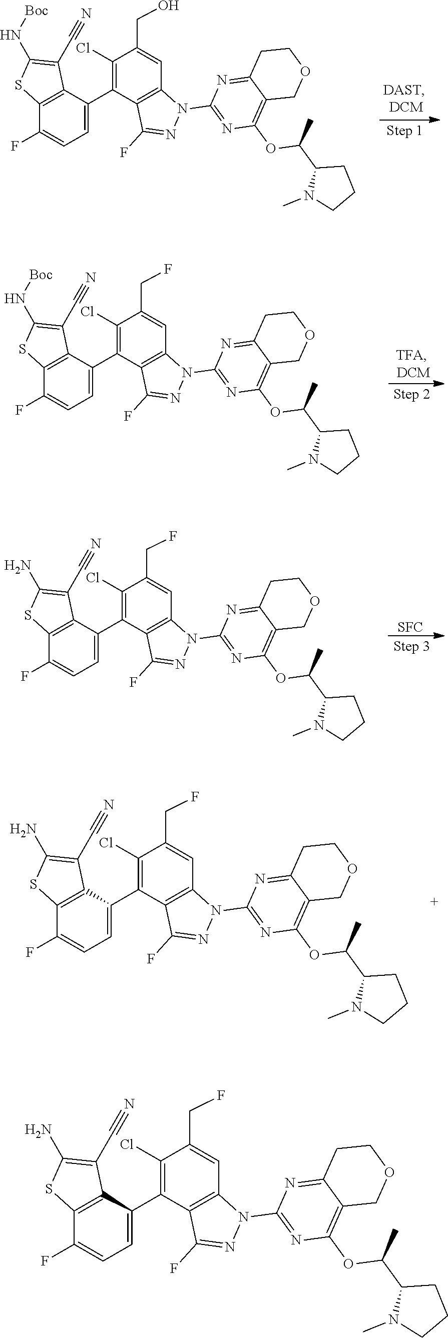

Step 1. Synthesis of tert-butyl N-[4-[5-chloro-3-fluoro-6-(fluoromethyl)-1-[4-[(1S)-1-[(2S)-1-methylpyrrolidin-2-yl]ethoxy]-7,8-dihydro-5H-pyrano[4,3-d]pyrimidin-2-yl]indazol-4-yl]-3-cyano-7-fluoro-benzothiophen-2-yl]carbamate

To a solution of Intermediate 38 (1 g, 1.33 mmol, 1 equiv) in DCM (20 mL) was added DAST (225 mg, 1.40 mmol) and stirred at 0° C. for 3 hr. The reaction mixture was quenched by addition water (60 mL) and then extracted with DCM (50 mL×3). The combined organic layers were dried over Na₂SO₄, filtered and concentrated under reduced pressure. The residue was purified by Prep-HPLC (column: Phenomenex luna C18 150*40 mm*15 um; mobile phase: [H2O(0.225% FA)-ACN]; gradient:38%-68% B over 15.0 min) to give the title compound (0.35 g, 34% yield) as yellow solid. 1H NMR (DMSO-d6, 400 MHz) δ 8.92 (br s, 1H), 7.4-7.4 (m, 1H), 7.25 (br t, 1H), 5.8-5.9 (m, 1H), 5.7-5.8 (m, 1H), 5.4-5.5 (m, 1H), 4.6-4.7 (m, 2H), 4.02 (br s, 2H), 2.95 (br d, 2H), 2.7-2.9 (m, 4H), 2.54 (s, 3H), 2.1-2.3 (m, 1H), 1.9-2.1 (m, 1H), 1.8-1.9 (m, 2H), 1.46 (s, 12H)

Step 2. Synthesis of 2-amino-4-[5-chloro-3-fluoro-6-(fluoromethyl)-1-[4-[(1S)-1-[(2S)-1-methylpyrrolidin-2-yl]ethoxy]-7,8-dihydro-5H-pyrano[4,3-d]pyrimidin-2-yl]indazol-4-yl]-7-fluoro-benzothiophene-3-carbonitrile

To a solution of the title compound in step 1 (0.3 g, 397 μmol) in DCM (2 mL) was added TFA (2.30 g, 20.19 mmol) and stirred at 30° C. for 1 hr. The reaction mixture was concentrated in vacuo. The residue was purified by Prep-HPLC (column: Phenomenex Luna C18 150*25 mm*10 um; mobile phase: [H2O(0.1% TFA)-ACN]; gradient:25%-55% B over 13.0 min) to give the title compound (220 mg, 84% yield) as a white solid. LCMS: m/z=654.2 [M+H]⁺

Step 3. Synthesis of (S)-2-amino-4-(5-chloro-3-fluoro-6-(fluoromethyl)-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile and (R)-2-amino-4-(5-chloro-3-fluoro-6-(fluoromethyl)-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

The title compound from step 2 (220 mg) was separated by SFC (column: DAICEL CHIRALPAK AD (250 mm*30 mm, 10 um); mobile phase: [CO2-EtOH (0.1% NH3H2O)]; B %: 35%, isocratic elution mode) to give Peak 1, Example 136, (S)-2-amino-4-(5-chloro-3-fluoro-6-(fluoromethyl)-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (R)-2-amino-4-(5-chloro-3-fluoro-6-(fluoromethyl)-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile (62.9 mg, 28% yield, 100% ee) as an off-white solid and

Peak 2, Example 137 (R)-2-amino-4-(5-chloro-3-fluoro-6-(fluoromethyl)-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (S)-2-amino-4-(5-chloro-3-fluoro-6-(fluoromethyl)-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile (117.9 mg, 45% yield, 99.1% ee) as an off-white solid.

### Peak 1, Example 136

LCMS: m/z=654.2 [M+H]⁺, ¹H NMR (400 MHz, DMSO-d₆) δ 8.92 (s, 1H), 8.09 (s, 2H), 7.35 (dd, 1H), 7.17 (t, 1H), 5.7-5.9 (m, 1H), 5.6-5.7 (m, 1H), 5.37 (t, 1H), 4.5-4.7 (m, 2H), 4.00 (t, 2H), 2.9-3.0 (m, 1H), 2.90 (br t, 2H), 2.6-2.7 (m, 1H), 2.37 (s, 3H), 2.24 (br d, 1H), 1.90 (br d, 1H), 1.69 (br dd, 3H), 1.34 (d, 3H)

### Peak 2, Example 137

LCMS: m/z=654.2 [M+H]⁺, 1H NMR (DMSO-d6, 400 MHz) δ 9.7-9.9 (m, 1H), 8.91 (s, 1H), 8.10 (s, 2H), 7.34 (dd, 1H), 7.18 (t, 1H), 5.8-5.9 (m, 1H), 5.72 (d, 1H), 5.40 (br dd, 1H), 4.6-4.7 (m, 2H), 4.0-4.1 (m, 2H), 3.7-3.9 (m, 1H), 3.5-3.6 (m, 1H), 3.1-3.2 (m, 1H), 2.97 (br d, 5H), 2.2-2.4 (m, 1H), 2.0-2.2 (m, 1H), 1.8-2.0 (m, 2H), 1.4-1.5 (m, 3H)

### Example 138

### (S)-2-amino-4-(5-chloro-3-fluoro-6-methyl-1-(5-methyl-4-((S)-1-((S)-1-methylpyrrolidin-2-yl)ethoxy)-6-oxo-5,6,7,8-tetrahydropyrido[3,2-d]pyrimidin-2-yl)-1H-indazol-4-yl)-7-fluorobenzo[b]thiophene-3-carbonitrile or (R)-2-amino-4-(5-chloro-3-fluoro-6-methyl-1-(5-methyl-4-((S)-1-((S)-1-methylpyrrolidin-2-yl)ethoxy)-6-oxo-5,6,7,8-tetrahydropyrido[3,2-d]pyrimidin-2-yl)-1H-indazol-4-yl)-7-fluorobenzo[b]thiophene-3-carbonitrile

### And Example 139

### (R)-2-amino-4-(5-chloro-3-fluoro-6-methyl-1-(5-methyl-4-((S)-1-((S)-1-methylpyrrolidin-2-yl)ethoxy)-6-oxo-5,6,7,8-tetrahydropyrido[3,2-d]pyrimidin-2-yl)-1H-indazol-4-yl)-7-fluorobenzo[b]thiophene-3-carbonitrile or (S)-2-amino-4-(5-chloro-3-fluoro-6-methyl-1-(5-methyl-4-((S)-1-((S)-1-methylpyrrolidin-2-yl)ethoxy)-6-oxo-5,6,7,8-tetrahydropyrido[3,2-d]pyrimidin-2-yl)-1H-indazol-4-yl)-7-fluorobenzo[b]thiophene-3-carbonitrile

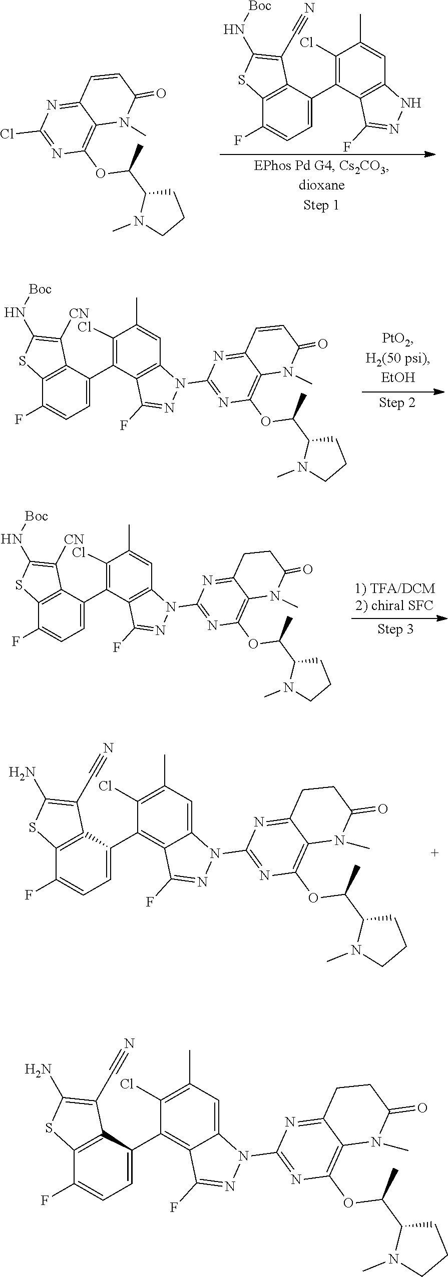

Step 1: Synthesis of tert-butyl (4-(5-chloro-3-fluoro-6-methyl-1-(5-methyl-4-((S)-1-((S)-1-methylpyrrolidin-2-yl)ethoxy)-6-oxo-5,6-dihydropyrido[3,2-d]pyrimidin-2-yl)-1H-indazol-4-yl)-3-cyano-7-fluorobenzo[b]thiophen-2-yl)carbamate

A mixture of 2-chloro-5-methyl-4-((S)-1-((S)-1-methylpyrrolidin-2-yl)ethoxy)pyrido[3,2-d]pyrimidin-6(5H)-one (239.6 mg, 504.54 μmol), the title compound from Examples 27 and 28, step 6 (190 mg, 458.67 μmol), Cs₂CO₃ (373.61 mg, 1.15 mmol) and EPhos Pd G4 (42.1 mg, 45.87 μmol) in dioxane (4 mL) was purged and degassed with N₂ and stirred at 80° C. for 1 h. The reaction mixture was purified by reversed-phase flash chromatography ([water (FA)-ACN]; gradient:10%-80% B, 9 min) to give the title compound (200 mg, 51.55% yield) as a white solid. LCMS: m/z=761.2 [M+H]⁺,

Step 2: Synthesis of tert-butyl (4-(5-chloro-3-fluoro-6-methyl-1-(5-methyl-4-((S)-1-((S)-1-methylpyrrolidin-2-yl)ethoxy)-6-oxo-5,6,7,8-tetrahydropyrido[3,2-d]pyrimidin-2-yl)-1H-indazol-4-yl)-3-cyano-7-fluorobenzo[b]thiophen-2-yl)carbamate

A suspension of the title compound from step 1 (90 mg, 118.23 μmol) and PtO₂ (90.0 mg, 396.34 μmol) in EtOH (5 mL) was stirred at 25° C. for 2 h under H₂ (50 psi) atmosphere. The mixture was filtered; the filtrate was concentrated under reduced pressure to give the title compound (70 mg, 89.56% yield) as white solid.-LCMS: m/z=762.2 [M+H]⁺

Step 3: Synthesis of (S)-2-amino-4-(5-chloro-3-fluoro-6-methyl-1-(5-methyl-4-((S)-1-((S)-1-methylpyrrolidin-2-yl)ethoxy)-6-oxo-5,6,7,8-tetrahydropyrido[3,2-d]pyrimidin-2-yl)-1H-indazol-4-yl)-7-fluorobenzo[b]thiophene-3-carbonitrile And (R)-2-amino-4-(5-chloro-3-fluoro-6-methyl-1-(5-methyl-4-((S)-1-((S)-1-methylpyrrolidin-2-yl)ethoxy)-6-oxo-5,6,7,8-tetrahydropyrido[3,2-d]pyrimidin-2-yl)-1H-indazol-4-yl)-7-fluorobenzo[b]thiophene-3-carbonitrile

A mixture of title compound from step 2 (80 mg, 104.81 μmol), TFA (3.07 g, 26.92 mmol, 2 mL) in DCM (4 mL) was stirred at 25° C. for 10 min. The reaction mixture was concentrated under reduced pressure and purified by reversed-phase HPLC (column: Phenomenex Luna C18 150*25 mm*10 um; mobile phase: [H2O(0.225% FA)-ACN]; gradient:22%-52% B over 15.0 min) to give racemic product. The racemic product was separated by chiral SFC (column: DAICEL CHIRALPAK IG (250 mm*30 mm, 10 um); mobile phase: [CO2-EtOH (0.1% NH3H2O)]; B %: 55%, isocratic elution mode) to give peak 1 and peak 2.

Peak 1 was further purified by reversed-phase HPLC (column: Phenomenex Luna C18 150*25 mm*10 um; mobile phase: [H2O(0.225% FA)-ACN]; gradient:22%-52% B over 15.0 min) to give the title compound, Example 138, (S)-2-amino-4-(5-chloro-3-fluoro-6-methyl-1-(5-methyl-4-((S)-1-((S)-1-methylpyrrolidin-2-yl)ethoxy)-6-oxo-5,6,7,8-tetrahydropyrido[3,2-d]pyrimidin-2-yl)-1H-indazol-4-yl)-7-fluorobenzo[b]thiophene-3-carbonitrile or (R)-2-amino-4-(5-chloro-3-fluoro-6-methyl-1-(5-methyl-4-((S)-1-((S)-1-methylpyrrolidin-2-yl)ethoxy)-6-oxo-5,6,7,8-tetrahydropyrido[3,2-d]pyrimidin-2-yl)-1H-indazol-4-yl)-7-fluorobenzo[b]thiophene-3-carbonitrile (9.1 mg, 18.20% yield) as a white solid. LCMS: m/z=662.2 [M+H]⁺1H NMR (400 MHz, 6d-DMSO) δ 8.70 (s, 1H), 8.33 (s, 1H), 8.05 (s, 2H), 7.32 (dd, 1H), 7.15 (t, 1H), 5.59-5.47 (m, 1H), 3.29 (s, 3H), 3.12-3.06 (m, 2H), 3.02-2.95 (m, 1H), 2.77-2.70 (m, 1H), 2.67 (br t,2H), 2.59 (s, 3H), 2.37 (s, 3H), 2.21 (dt, 1H), 1.90-1.79 (m, 2H), 1.75-1.61 (m, 2H), 1.38 (d, 3H). SFC: (100% purity, Rt=1.572 min)

Peak 2 was purified by reversed-phase HPLC (column: Phenomenex Luna C18 150*25 mm*10 um; mobile phase: [H2O(0.225% FA)-ACN]; gradient:22%-52% B over 15.0 min) to give the title compound, Example 139, (13.4 mg, 26.80% yield) as a white solid. LCMS: m/z=662.2 [M+H]⁺ 1H NMR (400 MHz, 6d-DMSO) δ 8.70 (s, 1H), 8.27 (s, 1H), 8.05 (s, 2H), 7.32 (dd, 1H), 7.21-7.12 (m, 1H), 5.60-5.47 (m, 1H), 3.29 (s, 3H), 3.12-3.06 (m, 2H), 3.01-2.95 (m, 1H), 2.74-2.64 (m, 3H), 2.59 (s, 3H), 2.37 (s, 3H), 2.24-2.17 (m, 1H), 1.88-1.78 (m, 2H), 1.75-1.62 (m, 2H), 1.39 (d, 3H). SFC: (100% purity, Rt=2.091 min)

### Example 140

### (S)-2-amino-4-(5,6-dichloro-3-fluoro-1-(4-((S)-1-((S)-1-methylpyrrolidin-2-yl)ethoxy)-6,7-dihydro-9H-[1,4]dioxepino[6,5-d]pyrimidin-2-yl)-1H-indazol-4-yl)-7-fluorobenzo[b]thiophene-3-carbonitrile or (R)-2-amino-4-(5,6-dichloro-3-fluoro-1-(4-((S)-1-((S)-1-methylpyrrolidin-2-yl)ethoxy)-6,7-dihydro-9H-[1,4]dioxepino[6,5-d]pyrimidin-2-yl)-1H-indazol-4-yl)-7-fluorobenzo[b]thiophene-3-carbonitrile

### And Example 141

### (R)-2-amino-4-(5,6-dichloro-3-fluoro-1-(4-((S)-1-((S)-1-methylpyrrolidin-2-yl)ethoxy)-6,7-dihydro-9H-[1,4]dioxepino[6,5-d]pyrimidin-2-yl)-1H-indazol-4-yl)-7-fluorobenzo[b]thiophene-3-carbonitrile or (S)-2-amino-4-(5,6-dichloro-3-fluoro-1-(4-((S)-1-((S)-1-methylpyrrolidin-2-yl)ethoxy)-6,7-dihydro-9H-[1,4]dioxepino[6,5-d]pyrimidin-2-yl)-1H-indazol-4-yl)-7-fluorobenzo[b]thiophene-3-carbonitrile

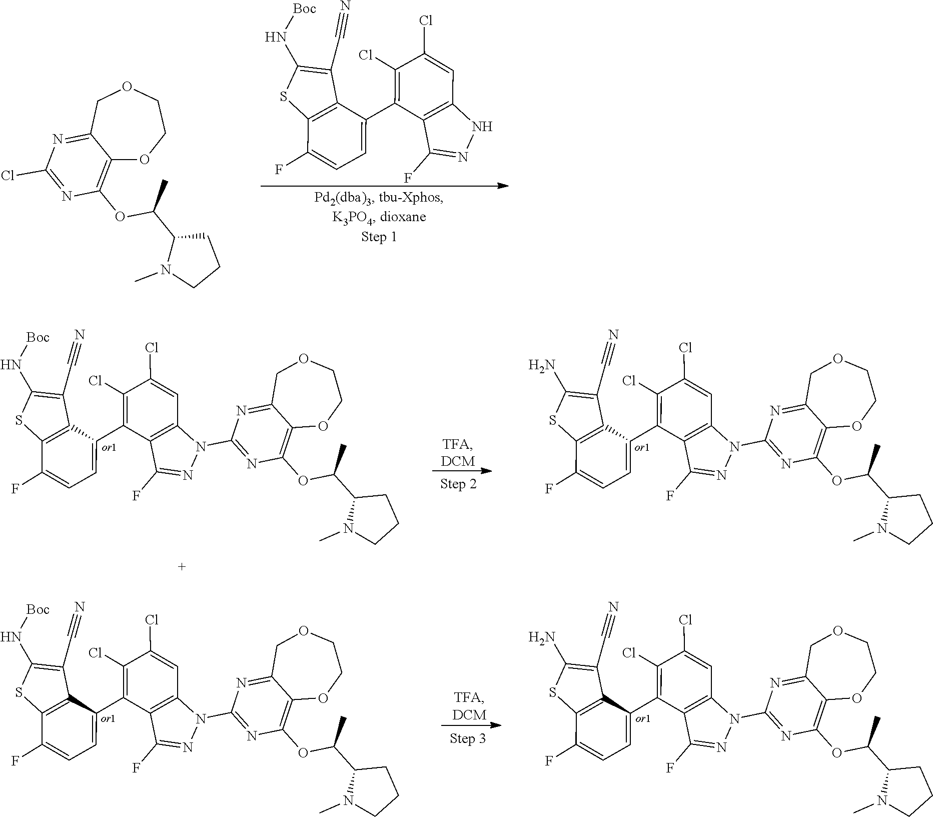

Step 1: Synthesis of tert-butyl ((S)-3-cyano-4-(5,6-dichloro-3-fluoro-1-(4-((S)-1-((S)-1-methylpyrrolidin-2-yl)ethoxy)-6,7-dihydro-9H-[1,4]dioxepino[6,5-d]pyrimidin-2-yl)-1H-indazol-4-yl)-7-fluorobenzo[b]thiophen-2-yl)carbamate and tert-butyl ((R)-3-cyano-4-(5,6-dichloro-3-fluoro-1-(4-((S)-1-((S)-1-methylpyrrolidin-2-yl)ethoxy)-6,7-dihydro-9H-[1,4]dioxepino[6,5-d]pyrimidin-2-yl)-1H-indazol-4-yl)-7-fluorobenzo[b]thiophen-2-yl)carbamate

A mixture of Intermediate 26 (150 mg, 417 μmol, FA), Intermediate 11 (206 mg, 417 μmol), potassium phosphate (265 mg, 1.25 mmol), Pd₂(dba)₃ (38.2 mg, 41.7 μmol) and di-tert-butyl-[2-(2,4,6-triisopropylphenyl)phenyl]phosphane (17.7 mg, 41.7 μmol) in dioxane (3 mL) was degassed and purged with N₂, stirred at 80° C. for 3 h under N₂. The mixture was diluted with water (2 mL) and extracted with EtOAc (2 mL×3). The combined organic layers were concentrated under reduced pressure. The residue was purified by prep-HPLC (column: Phenomenex Luna C18 150×25 mm×10 um; mobile phase: [H₂O (0.1% trifluoroacetic acid)-ACN]; gradient:33%-63% B over 15.0 min) to give fraction 1 (44 mg, 14% yield) LCMS: m/z=772.1 [M+H]⁺ and fraction 2 (30 mg, 9% yield) LCMS: m/z=772.1 [M+H]⁺ as white solids.

Step 2 and 3: Synthesis of (S)-2-amino-4-(5,6-dichloro-3-fluoro-1-(4-((S)-1-((S)-1-methylpyrrolidin-2-yl)ethoxy)-6,7-dihydro-9H-[1,4]dioxepino[6,5-d]pyrimidin-2-yl)-1H-indazol-4-yl)-7-fluorobenzo[b]thiophene-3-carbonitrile and (R)-2-amino-4-(5,6-dichloro-3-fluoro-1-(4-((S)-1-((S)-1-methylpyrrolidin-2-yl)ethoxy)-6,7-dihydro-9H-[1,4]dioxepino[6,5-d]pyrimidin-2-yl)-1H-indazol-4-yl)-7-fluorobenzo[b]thiophene-3-carbonitrile

To a solution of Fraction 1 from step 1 (34 mg, 44.00 μmol) in DCM (1 mL) was added TFA (460.50 mg, 4.04 mmol, 0.3 mL). The mixture was stirred at 25° C. for 1 h. The reaction mixture was concentrated under reduced pressure then purified by prep-HPLC (column: Phenomenex Luna C18 150×25 mm×10 um; mobile phase: [H2O (0.1% TFA)-ACN]; gradient:23%-53% B over 15.0 min) to give the title compound, Example 140 (30.9 mg, 35% yield) as a white solid.

LCMS: m/z=672.1 [M+H]⁺ 1H NMR (400 MHz, CD₃OD) δ ppm 8.97 (d, 1H), 7.29 (dd, 1H), 7.14-6.96 (m, 1H), 5.48 (dd, 1H), 4.90 (d, 2H), 4.26 (d, 2H), 4.09 (s, 2H), 3.89 (q,1H), 3.78-3.64 (m, 1H), 3.29-3.23 (m, 1H), 3.17 (s, 3H), 2.55-2.41 (m, 1H), 2.31-2.17 (m, 1H), 2.13 (td, 1H), 2.05-1.91 (m, 1H), 1.68-1.58 (m, 3H)

To a solution of Fraction 2 from step 1 (30.0 mg, 38.8 μmol) in DCM (1 mL) was added TFA (461 mg, 4.04 mmol, 0.3 mL). The mixture was stirred at 25° C. for 1 h. The reaction mixture was monitored by LCMS. The reaction mixture was concentrated under reduced pressure then purified by prep-HPLC (column: Phenomenex Luna C18 150×25 mm×10 um; mobile phase: [H2O (0.1% TFA)-ACN]; gradient:23%-53% B over 15.0 min) to give the title compound, Example 141, (18.1 mg, 69% yield) as an off-white solid.

LCMS: m/z=672.1 [M+H]⁺ 1H NMR (400 MHz, CD₃OD) δ ppm 8.98 (s, 1H), 7.28 (dd, 1H), 7.06 (t, 1H), 5.59-5.38 (m, 1H), 4.99-4.87 (m, 2H), 4.34-4.18 (m, 2H), 4.16-4.02 (m, 2H), 3.88 (q, 1H), 3.79-3.62 (m, 1H), 3.30-3.23 (m, 1H), 3.17 (s, 3H), 2.48 (qd, 1H), 2.31-2.17 (m, 1H), 2.17-2.05 (m, 1H), 2.05-1.92 (m, 1H), 1.63 (d, 3H)

### Example 142

### (S)-2-amino-4-(5-chloro-3-fluoro-6-methoxy-1-(4-((S)-1-((S)-1-methylpyrrolidin-2-yl)ethoxy)-6,7-dihydro-9H-[1,4]dioxepino[6,5-d]pyrimidin-2-yl)-1H-indazol-4-yl)-7-fluorobenzo[b]thiophene-3-carbonitrile or (R)-2-amino-4-(5-chloro-3-fluoro-6-methoxy-1-(4-((S)-1-((S)-1-methylpyrrolidin-2-yl)ethoxy)-6,7-dihydro-9H-[1,4]dioxepino[6,5-d]pyrimidin-2-yl)-1H-indazol-4-yl)-7-fluorobenzo[b]thiophene-3-carbonitrile

### And Example 143

### (R)-2-amino-4-(5-chloro-3-fluoro-6-methoxy-1-(4-((S)-1-((S)-1-methylpyrrolidin-2-yl)ethoxy)-6,7-dihydro-9H-[1,4]dioxepino[6,5-d]pyrimidin-2-yl)-1H-indazol-4-yl)-7-fluorobenzo[b]thiophene-3-carbonitrile or (S)-2-amino-4-(5-chloro-3-fluoro-6-methoxy-1-(4-((S)-1-((S)-1-methylpyrrolidin-2-yl)ethoxy)-6,7-dihydro-9H-[1,4]dioxepino[6,5-d]pyrimidin-2-yl)-1H-indazol-4-yl)-7-fluorobenzo[b]thiophene-3-carbonitrile

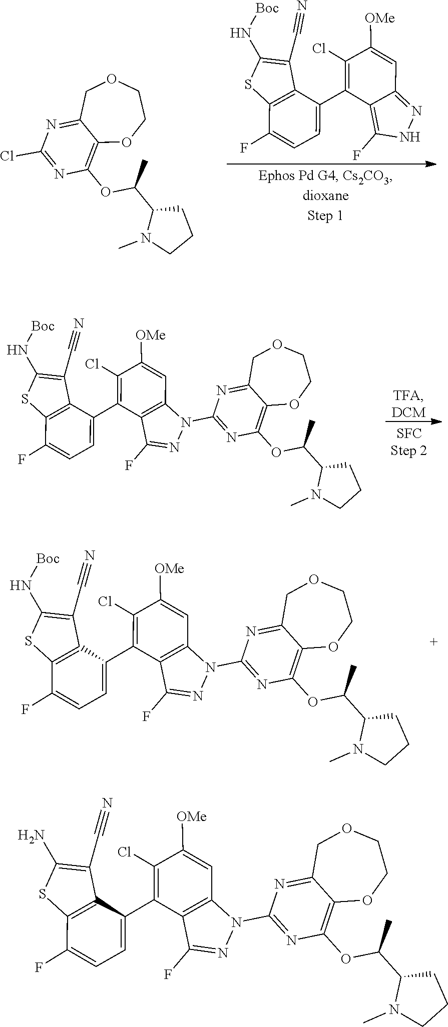

Step 1: Synthesis of tert-butyl (4-(5-chloro-3-fluoro-6-methoxy-1-(4-((S)-1-((S)-1-methylpyrrolidin-2-yl)ethoxy)-6,7-dihydro-9H-[1,4]dioxepino[6,5-d]pyrimidin-2-yl)-1H-indazol-4-yl)-3-cyano-7-fluorobenzo[b]thiophen-2-yl)carbamate

A mixture of Intermediate 26 (180 mg, 573 μmol), Intermediate 35 (281 mg, 573 μmol), EPhos Pd G4 (52.6 mg, 57.3 μmol) and Cs₂CO₃ (373 mg, 1.15 mmol) in dioxane (5 mL) was degassed and purged with N₂ then stirred at 80° C. for 0.5 h under N₂. The reaction mixture was filtered and concentrated under reduced pressure then purified by prep-HPLC (column: Phenomenex Luna C18 150*25 mm*10 um; mobile phase: [water (0.1% trifluoroacetic acid)-acetonitrile]; gradient:30%-60% B over 15.0 min) to give the title compound (360 mg, 80.0% yield) as a yellow solid. LCMS: m/z=768.4 [M+H]⁺

Step 2: (S)-2-amino-4-(5-chloro-3-fluoro-6-methoxy-1-(4-((S)-1-((S)-1-methylpyrrolidin-2-yl)ethoxy)-6,7-dihydro-9H-[1,4]dioxepino[6,5-d]pyrimidin-2-yl)-1H-indazol-4-yl)-7-fluorobenzo[b]thiophene-3-carbonitrile and (R)-2-amino-4-(5-chloro-3-fluoro-6-methoxy-1-(4-((S)-1-((S)-1-methylpyrrolidin-2-yl)ethoxy)-6,7-dihydro-9H-[1,4]dioxepino[6,5-d]pyrimidin-2-yl)-1H-indazol-4-yl)-7-fluorobenzo[b]thiophene-3-carbonitrile

To a solution of the title compound from step 1 (340 mg, 442 μmol) in DCM (3 mL) was added TFA (1.54 g, 13.4 mmol). The mixture was stirred at 25° C. for 0.5 h then concentrated under reduced pressure and purified by prep-HPLC (column: Phenomenex Luna C18 150*25 mm*10 um; mobile phase: [water (0.1% trifluoroacetic acid)-acetonitrile]; gradient:18%-48% B over 15.0 min) to give:

Peak 1, Example 142, (S)-2-amino-4-(5-chloro-3-fluoro-6-methoxy-1-(4-((S)-1-((S)-1-methylpyrrolidin-2-yl)ethoxy)-6,7-dihydro-9H-[1,4]dioxepino[6,5-d]pyrimidin-2-yl)-1H-indazol-4-yl)-7-fluorobenzo[b]thiophene-3-carbonitrile or (R)-2-amino-4-(5-chloro-3-fluoro-6-methoxy-1-(4-((S)-1-((S)-1-methylpyrrolidin-2-yl)ethoxy)-6,7-dihydro-9H-[1,4]dioxepino[6,5-d]pyrimidin-2-yl)-1H-indazol-4-yl)-7-fluorobenzo[b]thiophene-3-carbonitrile (110 mg, 37.3% yield) as a yellow solid LCMS: m/z=668.1 [M+H]^{+ 1}H NMR (400 MHz, 6d-DMSO) δ ppm 10.26-9.82 (m, 1H), 8.27 (d, 1H), 8.06 (s, 2H), 7.32-7.24 (m, 1H), 7.15 (t, 1H), 5.47-5.26 (m, 1H), 4.97-4.78 (m, 2H), 4.35-4.25 (m, 1H), 4.23-4.14 (m, 1H), 4.09 (s, 3H), 4.07-3.95 (m, 2H), 3.91-3.80 (m, 1H), 3.62-3.57 (m, 1H), 3.23-3.12 (m, 1H), 3.06 (d, 3H), 2.35-2.25 (m, 1H), 2.16-2.06 (m, 1H), 2.00-1.81 (m, 2H), 1.54 (d, 3H).

Peak 2, Example 143, (R)-2-amino-4-(5-chloro-3-fluoro-6-methoxy-1-(4-((S)-1-((S)-1-methylpyrrolidin-2-yl)ethoxy)-6,7-dihydro-9H-[1,4]dioxepino[6,5-d]pyrimidin-2-yl)-1H-indazol-4-yl)-7-fluorobenzo[b]thiophene-3-carbonitrile or (S)-2-amino-4-(5-chloro-3-fluoro-6-methoxy-1-(4-((S)-1-((S)-1-methylpyrrolidin-2-yl)ethoxy)-6,7-dihydro-9H-[1,4]dioxepino[6,5-d]pyrimidin-2-yl)-1H-indazol-4-yl)-7-fluorobenzo[b]thiophene-3-carbonitrile (104 mg, 35.2% yield, 92% ee) as a yellow solid. LCMS: m/z=668.1 [M+H]^{+ 1}H NMR (400 MHz, 6d-DMSO) δ ppm 10.08 (s, 1H), 8.25 (d, 1H), 8.07 (s, 2H), 7.32-7.24 (m, 1H), 7.15 (t, 1H), 5.44-5.25 (m, 1H), 4.95-4.81 (m, 2H), 4.35-4.24 (m, 1H), 4.23-4.15 (m, 1H), 4.12-3.99 (m, 5H), 3.89-3.77 (m, 1H), 3.61-3.54 (m, 1H), 3.22-3.11 (m, 1H), 3.05 (d 3H), 2.35-2.21 (m, 1H), 2.14-2.05 (m, 1H), 2.00-1.83 (m, 2H), 1.54 (d, 3H)

### Example 144

### (R)-2-amino-4-(5-chloro-3-fluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorothieno[3,2-c]pyridine-3-carbonitrile or (S)-2-amino-4-(5-chloro-3-fluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorothieno[3,2-c]pyridine-3-carbonitrile

### And Example 145

### (S)-2-amino-4-(5-chloro-3-fluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorothieno[3,2-c]pyridine-3-carbonitrile or (R)-2-amino-4-(5-chloro-3-fluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorothieno[3,2-c]pyridine-3-carbonitrile

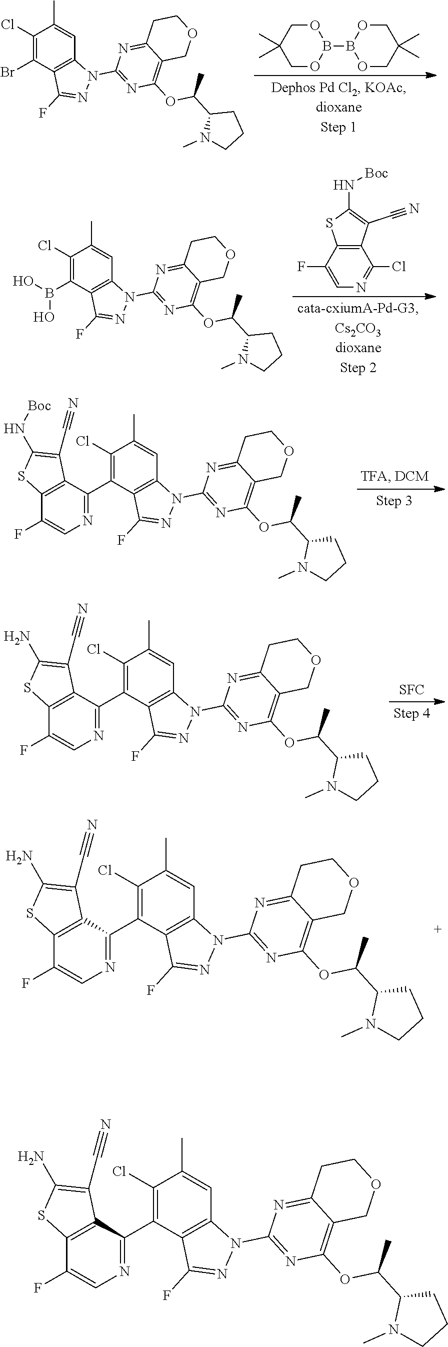

Step 1: Synthesis of (5-chloro-3-fluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)boronic acid

A mixture of Intermediate 39 (100 mg, 0.19 mmol), 2-(5,5-dimethyl-1,3,2-dioxaborinan-2-yl)-5,5-dimethyl-1,3,2-dioxaborinane (172 mg, 0.76 mmol), potassium acetate (56 mg, 0.57 mmol), DPEphosPdCl₂ (14 mg, 0.02 mmol) in dioxane (2 mL) was degassed and purged with N₂ for 3 times, and then the mixture was stirred at 100° C. for 2 h under N₂. The reaction mixture was concentrated under reduced pressure. The crude product was purified by reversed-phase HPLC [water (0.1% formic acid)-acetonitrile]; B %: 60%-80%, 7 min) to give the title compound (90 mg, 96% yield) as a white solid. LCMS: m/z=490.0 [M+H]⁺.

Step 2: Synthesis of tert-butyl (4-(5-chloro-3-fluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-3-cyano-7-fluorothieno[3,2-c]pyridin-2-yl)carbamate

A mixture of the title compound from Step 1 (80 mg, 0.16 mmol), tert-butyl N-(4-chloro-3-cyano-7-fluoro-thieno[3,2-c]pyridin-2-yl)carbamate (54 mg, 0.16 mmol), Cs₂CO₃ (106 mg, 0.33 mmol), cataCXium A Pd G3 (12 mg, 0.016 mmol) in dioxane (5 mL) was degassed and purged with N₂ for 3 times, and then the mixture was stirred at 110° C. for 2 h under N₂ in a microwave reactor. The reaction mixture was concentrated under reduced pressure. The residue was purified by prep-HPLC (column: Phenomenex Luna C18 150×25 mm×10 um; mobile phase: [water (0.225% formic acid)-acetonitrile]; gradient:32%-62% B over 10.0 min) to give the title compound (50 mg, 42% yield) as a white solid. LCMS: m/z=737.4 [M+H]⁺.

Step 3: Synthesis of 2-amino-4-(5-chloro-3-fluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorothieno[3,2-c]pyridine-3-carbonitrile

To a solution of title compound from step 2 (45 mg, 0.06 mmol) in DCM (1 mL) was added TFA (1.38 g, 12 mmol, 0.9 mL). The mixture was stirred at 25° C. for 0.5 h then concentrated under reduced pressure. The crude product was purified by reversed-phase HPLC [water (0.1% formic acid)-acetonitrile]; B %: 30%-50%, 7 min) to give the title compound (30 mg, 77% yield) as a white solid. LCMS: m/z=637.2 [M+H]⁺.

Step 4: Synthesis of (S)-2-amino-4-(5-chloro-3-fluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorothieno[3,2-c]pyridine-3-carbonitrile and (R)-2-amino-4-(5-chloro-3-fluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorothieno[3,2-c]pyridine-3-carbonitrile

The title compound from step 3 (30 mg, 0.47 mmol) was separated by SFC (column: DAICEL CHIRALPAK AD (250 mm×30 mm, 10 um); mobile phase: [CO2-isopropanol: acetonitrile=4:1 (0.1% ammonium hydroxide)]; B %: 45%, isocratic elution mode) and purified by prep-HPLC (column: Phenomenex Luna C18 150×25 mm×10 um; mobile phase: [water(0.225% formic acid)-acetonitrile]; gradient:18%-48% B over 15.0 min) to give Peak 1, Example 144 (3.0 mg, 10% yield) as an off-white solid and Peak 2, Example 145 (3.0 mg, 10% yield) as an off-white solid.

Peak 1, Example 144, LCMS: m/z=637.3 [M+H]⁺. ¹H NMR (400 MHz, 6d-DMSO): δ=8.77 (s, 1H), 8.48-8.38 (m, 3H), 5.41 (t, 1H), 4.72-4.49 (m, 2H), 4.01 (t, 2H), 3.00-2.89 (m, 3H), 2.67-2.62 (m, 1H), 2.60 (s, 3H), 2.37 (s, 3H), 2.28-2.15 (m, 1H), 1.94-1.81 (m, 1H), 1.78-1.60 (m, 3H), 1.34 (d, 3H)

Peak 2, Example 145, LCMS: m/z=637.3 [M+H]⁺. ¹H NMR (400 MHz, 6d-DMSO): δ=8.77 (s, 1H), 8.45 (s, 3H), 5.42 (t, 1H), 4.69-4.50 (m, 2H), 4.00 (t, 2H), 2.99-2.90 (m, 3H), 2.67-2.61 (m, 1H), 2.60 (s, 3H), 2.36 (s, 3H), 2.26-2.16 (m, 1H), 1.90-1.78 (m, 1H), 1.77-1.61 (m, 3H), 1.35 (d, 3H)

### Example 146

### (R)-2-amino-4-(5-chloro-3-fluoro-1-(4-((S)-1-((S)-1-methylpyrrolidin-2-yl)ethoxy)-7,8-dihydro-5H-pyrano[4,3-d]pyrimidin-2-yl)-1H-pyrazolo[3,4-c]pyridin-4-yl)-7-fluorobenzo[b]thiophene-3-carbonitrile or (S)-2-amino-4-(5-chloro-3-fluoro-1-(4-((S)-1-((S)-1-methylpyrrolidin-2-yl)ethoxy)-7,8-dihydro-5H-pyrano[4,3-d]pyrimidin-2-yl)-1H-pyrazolo[3,4-c]pyridin-4-yl)-7-fluorobenzo[b]thiophene-3-carbonitrile

### And Example 147

### (S)-2-amino-4-(5-chloro-3-fluoro-1-(4-((S)-1-((S)-1-methylpyrrolidin-2-yl)ethoxy)-7,8-dihydro-5H-pyrano[4,3-d]pyrimidin-2-yl)-1H-pyrazolo[3,4-c]pyridin-4-yl)-7-fluorobenzo[b]thiophene-3-carbonitrile or (R)-2-amino-4-(5-chloro-3-fluoro-1-(4-((S)-1-((S)-1-methylpyrrolidin-2-yl)ethoxy)-7,8-dihydro-5H-pyrano[4,3-d]pyrimidin-2-yl)-1H-pyrazolo[3,4-c]pyridin-4-yl)-7-fluorobenzo[b]thiophene-3-carbonitrile

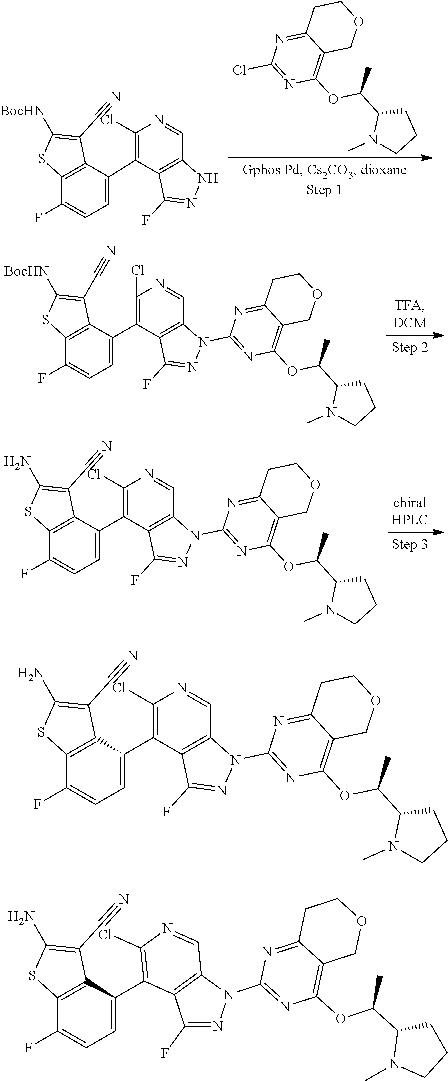

Step 1: Synthesis of tert-butyl (4-(5-chloro-3-fluoro-1-(4-((S)-1-((S)-1-methylpyrrolidin-2-yl)ethoxy)-7,8-dihydro-5H-pyrano[4,3-d]pyrimidin-2-yl)-1H-pyrazolo[3,4-c]pyridin-4-yl)-3-cyano-7-fluorobenzo[b]thiophen-2-yl)carbamate:

A mixture of Intermediate 40 (90 mg, 194 μmol), Intermediate 1 (86.6 mg, 291 μmol), Cs₂CO₃ (126 mg, 388 μmol) and Gphos Pd G6 (18.3 mg, 19.4 μmol) in dioxane (3 mL) was stirred at 100° C. for 3 h under N₂.

The reaction solution was poured into water (10 mL) and extracted with EtOAc (10 mL×3). The organic layer was washed with brine (10 mL), dried over Na₂SO₄ and evaporated. The crude product was purified by Prep-TLC with DCM:MeOH=10:1 to afford the title compound (100 mg, 71.4%) as a yellow solid. LCMS: m/z=723 [M+H]⁺

Step 2: Synthesis of 2-amino-4-(5-chloro-3-fluoro-1-(4-((S)-1-((S)-1-methylpyrrolidin-2-yl)ethoxy)-7,8-dihydro-5H-pyrano[4,3-d]pyrimidin-2-yl)-1H-pyrazolo[3,4-c]pyridin-4-yl)-7-fluorobenzo[b]thiophene-3-carbonitrile

A solution of the title compound from step 1 (95 mg, 131 μmol) in DCM (3 mL) was added TFA (1 mL) and the resulting mixture was stirred at rt for 3 h. The resulted solution was evaporated and purified using PREP-HPLC with following conditions: (Column: XBridge Prep OBD C18 Column, 30*150 mm, 5 μm; Mobile Phase A: Water (10 mmol/L NH₄HCO₃)+0.05% NH₃·H₂O, Mobile Phase B: ACN; Flow rate: 60 mL/min; Gradient (B %): 52% B to 83% B in 7 min; wavelength: 254 nm/220 nm) to afford the title compound (50 mg, 61.2%) as a white solid. LCMS: m/z=623 [M+H]⁺

Step 3: Synthesis of (S)-2-amino-4-(5-chloro-3-fluoro-1-(4-((S)-1-((S)-1-methylpyrrolidin-2-yl)ethoxy)-7,8-dihydro-5H-pyrano[4,3-d]pyrimidin-2-yl)-1H-pyrazolo[3,4-c]pyridin-4-yl)-7-fluorobenzo[b]thiophene-3-carbonitrile and (R)-2-amino-4-(5-chloro-3-fluoro-1-(4-((S)-1-((S)-1-methylpyrrolidin-2-yl)ethoxy)-7,8-dihydro-5H-pyrano[4,3-d]pyrimidin-2-yl)-1H-pyrazolo[3,4-c]pyridin-4-yl)-7-fluorobenzo[b]thiophene-3-carbonitrile

The title compound from step 2 (50 mg, 80.2 μmol) in MeOH (2 mL) was separated by PREP-CHIRAL-HPLC with following conditions: (Column: CHIRALPAK IK 3*25 cm, 5 μm; Mobile Phase A: Hex (0.5% 2M NH₃-MeOH)—HPLC, Mobile Phase B: ETOH: DCM=1:1-HPLC; Flow rate: 40 mL/min; Gradient (B %): 30% B; wavelength: 220/254 nm) to afford Peak 1, Example 146, (R)-2-amino-4-(5-chloro-3-fluoro-1-(4-((S)-1-((S)-1-methylpyrrolidin-2-yl)ethoxy)-7,8-dihydro-5H-pyrano[4,3-d]pyrimidin-2-yl)-1H-pyrazolo[3,4-c]pyridin-4-yl)-7-fluorobenzo[b]thiophene-3-carbonitrile or (S)-2-amino-4-(5-chloro-3-fluoro-1-(4-((S)-1-((S)-1-methylpyrrolidin-2-yl)ethoxy)-7,8-dihydro-5H-pyrano[4,3-d]pyrimidin-2-yl)-1H-pyrazolo[3,4-c]pyridin-4-yl)-7-fluorobenzo[b]thiophene-3-carbonitrile (22.0 mg, 44.0%) as a yellow solid, and Peak 2, Example 147 (S)-2-amino-4-(5-chloro-3-fluoro-1-(4-((S)-1-((S)-1-methylpyrrolidin-2-yl)ethoxy)-7,8-dihydro-5H-pyrano[4,3-d]pyrimidin-2-yl)-1H-pyrazolo[3,4-c]pyridin-4-yl)-7-fluorobenzo[b]thiophene-3-carbonitrile or (R)-2-amino-4-(5-chloro-3-fluoro-1-(4-((S)-1-((S)-1-methylpyrrolidin-2-yl)ethoxy)-7,8-dihydro-5H-pyrano[4,3-d]pyrimidin-2-yl)-1H-pyrazolo[3,4-c]pyridin-4-yl)-7-fluorobenzo[b]thiophene-3-carbonitrile (9.4 mg, yield: 38.8%) as a white solid

Peak 1, Example 146, LCMS: m/z=623 [M+H]⁺, ¹H NMR (400 MHz, DMSO) δ 9.85 (d, 1H), 8.18 (s, 2H), 7.43 (dd, 1H), 7.20 (t, 1H), 5.48-5.40 (m, 1H), 4.62 (q, 2H), 4.04-4.00 (m, 2H), 3.36-3.30 (m, 2H), 3.00-2.95 (m, 3H), 2.64-2.50 (m, 1H), 2.50-2.31 (m, 2H), 1.83-1.78 (m, 4H), 1.40 (d, 3H).

Peak 2, Example 147, LCMS: m/z=623 [M+H]⁺, ¹H NMR (400 MHz, DMSO) δ 9.85 (d, 1H), 8.18 (s, 2H), 7.43 (dd, 1H), 7.20 (dd, 1H), 5.47-5.39 (m, 1H), 4.69-4.55 (m, 2H), 4.02 (t, 2H), 3.37-3.33 (m, 2H), 3.00-2.90 (m, 3H), 2.55-2.51 (m, 1H), 2.48-2.32 (m, 2H), 1.81-1.77 (m, 4H), 1.39 (d, 3H).

### Intermediate 148

### (S)-2-amino-4-(5-chloro-6-methoxy-1-(4-((S)-1-((S)-1-methylpyrrolidin-2-yl)ethoxy)-7,8-dihydro-5H-pyrano[4,3-d]pyrimidin-2-yl)-1H-benzo[d][1,2,3]triazol-4-yl)-7-fluorobenzo[b]thiophene-3-carbonitrile or (R)-2-amino-4-(5-chloro-6-methoxy-1-(4-((S)-1-((S)-1-methylpyrrolidin-2-yl)ethoxy)-7,8-dihydro-5H-pyrano[4,3-d]pyrimidin-2-yl)-1H-benzo[d][1,2,3]triazol-4-yl)-7-fluorobenzo[b]thiophene-3-carbonitrile

### And Intermediate 149

### (R)-2-amino-4-(5-chloro-6-methoxy-1-(4-((S)-1-((S)-1-methylpyrrolidin-2-yl)ethoxy)-7,8-dihydro-5H-pyrano[4,3-d]pyrimidin-2-yl)-1H-benzo[d][1,2,3]triazol-4-yl)-7-fluorobenzo[b]thiophene-3-carbonitrile or (S)-2-amino-4-(5-chloro-6-methoxy-1-(4-((S)-1-((S)-1-methylpyrrolidin-2-yl)ethoxy)-7,8-dihydro-5H-pyrano[4,3-d]pyrimidin-2-yl)-1H-benzo[d][1,2,3]triazol-4-yl)-7-fluorobenzo[b]thiophene-3-carbonitrile

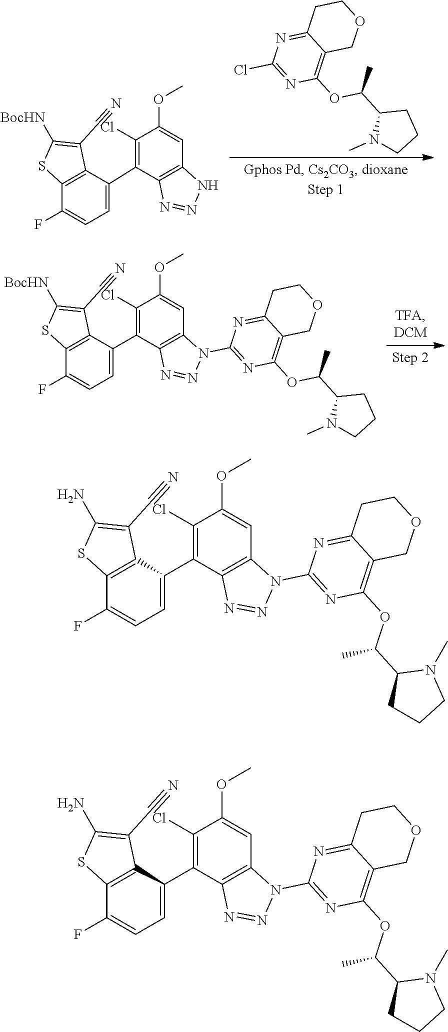

Step 1: Synthesis of tert-butyl (4-{5-chloro-6-methoxy-1-[4-({(1S)-1-[(2S)-1-methylpyrrolidin-2-yl]ethyl}oxy)-7,8-dihydro-5H-pyrano[4,3-d]pyrimidin-2-yl]-1H-benzotriazol-4-yl}-3-cyano-7-fluoro-1-benzothiophen-2-yl)carbamate

A mixture of Intermediate 41 (800 mg, 1.68 mmol), Intermediate 1 (752.8 mg, 2.528 mmol), GPhos Pd G6 (317.6 mg, 336.8 μmol) and Cs₂CO₃ (1.096 g, 3.376 mmol) in dioxane (10 mL) under N₂ was heated to 100° C. for 2 h. The mixture was then poured into H₂O (100 mL) and extracted with EtOAc (3×100 mL). The organic layer was washed with brine (50 mL), dried over Na₂SO₄ and evaporated. The crude product was purified by flash chromatography with DCM:MeOH=10:1 to afford the title compound (60 mg, 4.8%) as a yellow solid. LCMS: m/z=735 [M+H]⁺.

Step 2: Synthesis of (S)-2-amino-4-(5-chloro-6-methoxy-1-(4-((S)-1-((S)-1-methylpyrrolidin-2-yl)ethoxy)-7,8-dihydro-5H-pyrano[4,3-d]pyrimidin-2-yl)-1H-benzo[d][1,2,3]triazol-4-yl)-7-fluorobenzo[b]thiophene-3-carbonitrile and (R)-2-amino-4-(5-chloro-6-methoxy-1-(4-((S)-1-((S)-1-methylpyrrolidin-2-yl)ethoxy)-7,8-dihydro-5H-pyrano[4,3-d]pyrimidin-2-yl)-1H-benzo[d][1,2,3]triazol-4-yl)-7-fluorobenzo[b]thiophene-3-carbonitrile

To a solution of the title compound from step 1 (40 mg, 0.08 mmol) in DCM (3 mL) was added TFA (1 mL) at rt. The mixture was stirred for 2 h at rt. The mixture was concentrated and the residue was separated by Prep-Chiral-HPLC with the following conditions Column: CHIRALPAK IK, 5 μm,250 mm×20 mm; Mobile Phase A: Hex (with 0.5% NH3 (2 M in MeOH)), Mobile Phase B: 1:1 EtOH: DCM; Flow rate: 20 mL/min; Gradient (B %): isocratic 40% B; wavelength: 220/254 nm; RT1(min): 8.85; RT2(min): 12.44; Sample Solvent: EtOH; Injection Volume: 1.0 mL; Number Of Runs: 3; Single injection run time (min): 15 to afford, Peak 1, Example 148, (S)-2-amino-4-(5-chloro-6-methoxy-1-(4-((S)-1-((S)-1-methylpyrrolidin-2-yl) ethoxy)-7,8-dihydro-5H-pyrano[4,3-d]pyrimidin-2-yl)-1H-benzo[d][1,2,3]triazol-4-yl)-7-fluorobenzo[b]thiophene-3-carbonitrile or (R)-2-amino-4-(5-chloro-6-methoxy-1-(4-((S)-1-((S)-1-methylpyrrolidin-2-yl)ethoxy)-7,8-dihydro-5H-pyrano[4,3-d]pyrimidin-2-yl)-1H-benzo[d][1,2,3]triazol-4-yl)-7-fluorobenzo[b]thiophene-3-carbonitrile (10.5 mg, yield: 26%) as a white solid and Peak 2, Example 149, Synthesis of (R)-2-amino-4-(5-chloro-6-methoxy-1-(4-((S)-1-((S)-1-methylpyrrolidin-2-yl)ethoxy)-7,8-dihydro-5H-pyrano[4,3-d]pyrimidin-2-yl)-1H-benzo[d][1,2,3]triazol-4-yl)-7-fluorobenzo[b]thiophene-3-carbonitrile or (S)-2-amino-4-(5-chloro-6-methoxy-1-(4-((S)-1-((S)-1-methylpyrrolidin-2-yl)ethoxy)-7,8-dihydro-5H-pyrano[4,3-d]pyrimidin-2-yl)-1H-benzo[d][1,2,3]triazol-4-yl)-7-fluorobenzo[b]thiophene-3-carbonitrile (11.2 mg yield: 27%) as a white solid.

Peak 1, Example 148, LCMS: m/z=635 [M+H]⁺, ¹H NMR (300 MHz, DMSO) δ 8.15 (s, 1H), 8.02 (s, 2H), 7.32 (dd, 1H), 7.27-7.16 (m, 1H), 5.44 (t, 1H), 4.74-4.55 (m, 2H), 4.08 (s, 3H), 4.03 (t, 2H), 3.05-2.94 (m, 3H), 2.79-2.71 (m, 1H), 2.41 (s, 3H), 2.33-2.26 (m, 1H), 1.99-1.85 (m, 1H), 1.80-1.63 (m, 3H), 1.38 (d, 3H).

Peak 2, Example 149, LCMS: m/z=635 [M+H]⁺, ¹H NMR (300 MHz, DMSO) δ 8.13 (s, 1H), 8.02 (s, 2H), 7.32 (dd, 1H), 7.19 (dd, 1H), 5.43 (q, 1H), 4.77-4.52 (m, 2H), 4.08 (s, 3H), 4.03 (t, 2H), 3.03-2.92 (m, 3H), 2.70-2.61 (m, 1H), 2.36 (s, 3H), 2.29-2.19 (m, 1H), 1.92-1.84 (m, 1H), 1.73-1.62 (m, 3H), 1.38 (d, 3H).

### Example 150

### (S)-2-amino-4-(5-(difluoromethyl)-3-fluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (R)-2-amino-4-(5-(difluoromethyl)-3-fluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

### And Example 151

### (R)-2-amino-4-(5-(difluoromethyl)-3-fluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (S)-2-amino-4-(5-(difluoromethyl)-3-fluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

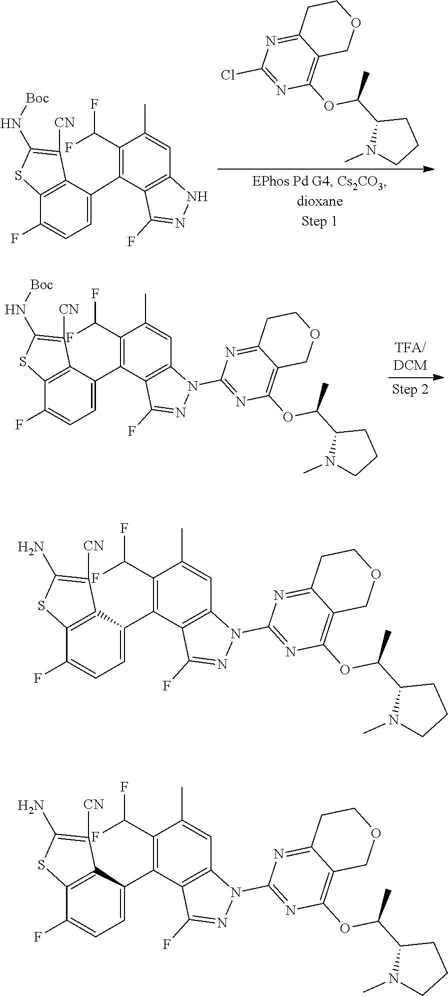

Step 1: Synthesis of tert-butyl (3-cyano-4-(5-(difluoromethyl)-3-fluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophen-2-yl)carbamate

To a solution of Intermediate 42 (100 mg, 203 μmol), Intermediate 1 (60.7 mg, 203 μmol), EPhos Pd G4 (8.73 mg, 20.3 μmol) and Cs₂CO₃ (32.8 mg, 406 mmol) in dioxane (2 mL) was degassed and purged with N₂ (3 times) then stirred at 80° C. for 1 h under N₂. The mixture was filtered and concentrated under reduced pressure. The residue was purified by prep-HPLC (column: Phenomenex Luna C18 150*25 mm*10 um; mobile phase: [H2O(0.1% TFA)-ACN]; gradient:30%-60% B over 15.0 min) to give compound the title compound (100 mg, 126 μmol, 61% yield) as a white solid. LCMS: m/z=752.3 [M+H]⁺

Step 2: Synthesis of (S)-2-amino-4-(5-(difluoromethyl)-3-fluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile and (R)-2-amino-4-(5-(difluoromethyl)-3-fluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

To a solution of the title compound from step 1 (90.0 mg, 119 μmol) in DCM (1.35 mL) was added TFA (690 mg, 6.06 mmol, 450 μL) and stirred at rt for 0.5 h. The mixture was then concentrated under reduced pressure to give a residue. The residue was purified by prep-HPLC (column: Phenomenex Luna C18 150*25 mm*10 um; mobile phase: [H2O(0.1% TFA)-ACN]; gradient:20%-50% B over 15.0 min) to give compound Peak 1, Example 150, (S)-2-amino-4-(5-(difluoromethyl)-3-fluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (R)-2-amino-4-(5-(difluoromethyl)-3-fluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile (7.70 mg, 10.0 μmol, 8% yield, TFA, 100% ee) as a white solid and Peak 2, Example 151, (R)-2-amino-4-(5-(difluoromethyl)-3-fluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (S)-2-amino-4-(5-(difluoromethyl)-3-fluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile (18.5 mg, 24.1 μmol, 20% yield, TFA, 91.2% ee) as a white solid.

Peak 1, Example 150 LCMS: m/z=652.2 [M+H]⁺, ¹H NMR (400 MHz, 6d-DMSO): δ ppm 9.99 (br d, 1H), 8.70 (s, 1H), 8.09 (s, 2H), 7.32-7.22 (m, 1H), 7.22-7.10 (m, 1H), 6.85-6.51 (m, 1H), 5.50-5.38 (m, 1H), 4.76-4.58 (m, 2H), 4.07-4.00 (m, 2H), 3.82-3.79 (m, 1H), 3.62-3.57 (m, 1H), 3.24-3.15 (m, 1H), 2.98 (br d, 5H), 2.75 (s, 3H), 2.39-2.25 (m, 1H), 2.16-2.07 (m, 1H), 2.04-1.87 (m, 2H), 1.52 (d, 3H).

LCMS data: M+1 (652.2); MW (651.2).

Peak 2, Example 151: LCMS: m/z=652.2 [M+H]⁺, ¹H NMR (400 MHz, 6d-DMSO): δ ppm 9.95 (br d, 1H), 8.67 (s, 1H), 8.09 (s, 2H), 7.28-7.22 (m, 1H), 7.20-7.14 (m, 1H), 6.81-6.50 (m, 1H), 5.49-5.37 (m, 1H), 4.74-4.58 (m, 2H), 4.07-4.01 (m, 2H), 3.81-3.78 (m, 1H), 3.59-3.53 (m, 1H), 3.24-3.14 (m, 1H), 3.04-2.92 (m, 5H), 2.74 (s, 3H), 2.34-2.24 (m, 1H), 2.15-2.05 (m, 1H), 2.00-1.87 (m, 2H), 1.51 (d, 3H).

### Example 152

### (S)-2-amino-4-(3,5-difluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (R)-2-amino-4-(3,5-difluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

### And Example 153

### (R)-2-amino-4-(3,5-difluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (S)-2-amino-4-(3,5-difluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol -4-yl)-7-fluorobenzo[b]thiophene-3-carbonitrile

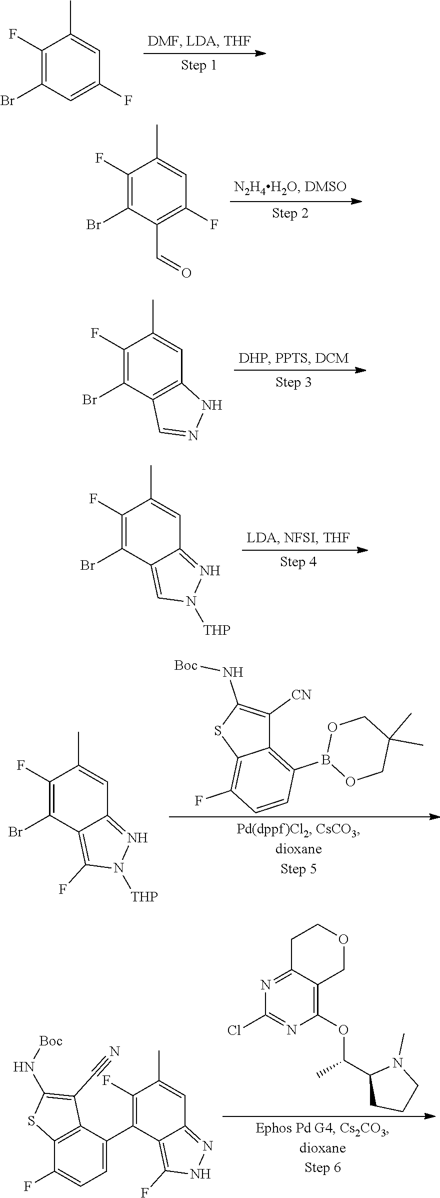

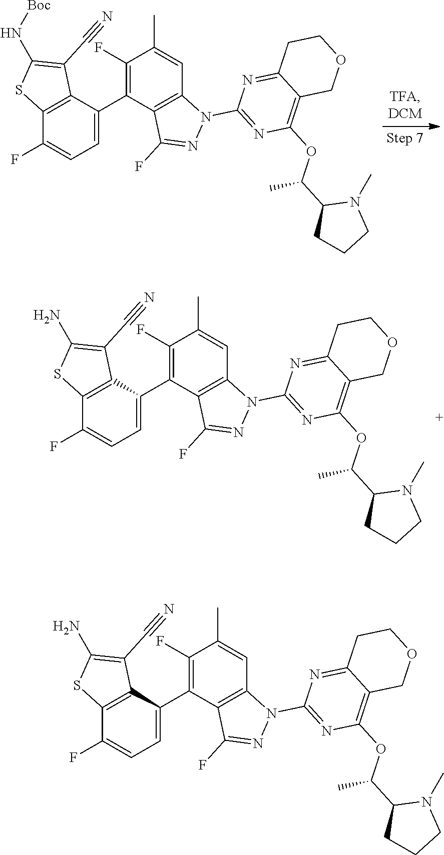

Step 1: Synthesis of 2-bromo-3,6-difluoro-4-methylbenzaldehyde

Solution 1:1-bromo-2,5-difluoro-3-methylbenzene (24.5 g, 118.35 mmol) in THF (400 mL)

Solution 2: LDA (1 M, 177.52 mL)

Solution 3: N,N-dimethylformamide (25.95 g, 355.05 mmol, 27.32 mL, 3 eq) in THF (400 mL)

Solution 1 was pumped by Pump 1 {S1, P1, 9.471 mL/min} to flow reactor1 {FLR1, PFA, Coils reactor, 1.590( 1/16″) mm, 0.636 mL, −20° C.}

Solution 2 was pumped by Pump 2 {S2, P2, 3.249 mL/min} to flow reactor1 {FLR1, PFA, Coils reactor, 1.590( 1/16″) mm, 0.636 mL, −20° C.}

Solution 3 was pumped by Pump 3 {S3, P3, 9.501 mL/min} to flow reactor2 {FLR2, PFA, Coils reactor, 1.590( 1/16″) mm, 0.636 mL, −20° C.}

The residence time of flow reactor 1 was {FLR1, 0.05 min}. The output of flow reactor 1 was pumped into flow reactor 2.

The residence time of flow reactor 2 was {FLR2, 0.029 min}.

All solutions were purged out within 2 h. The mixture was quenched with saturated aqueous NH₄Cl (100 mL) and extracted with EtOAc (200 mL). The organic phase was washed with brine (2×100 mL). The organic phase was dried over Na2SO4, filtered and concentrated in vacuo. The residue was purified by column chromatography (SiO2, PE: EtOAc=20/1 to 3/1) to give compound the title compound (24.6 g, 88.44% yield) as yellow solid. 1H NMR (400 MHz, CDCl3): ppm 10.29 (s, 1H), 7.03-6.99 (m, 1H), 2.393 (s, 3H)

Step 2: Synthesis of 4-bromo-5-fluoro-6-methyl-1H-indazole

Two batches of the reaction were performed as follows. To a solution of the title compound from step 2 (10 g, 42.55 mmol) in DMSO (150 mL) was added N₂H₄·H₂O (51.60 g, 1.03 mol, 50 mL, 24.23 equiv) at 20° C., and then the mixture was stirred at 20° C. for 1 h under N₂. The mixture was heated to 130° C. for 6 h under N₂. The reaction mixture was diluted with brine (50 mL) and extracted with EtOAc (3×100 mL). The combined organic layers were washed with saturated brine (3×50 mL), dried over Na₂SO₄, filtered and concentrated under reduced pressure to give a solid. The solid was purified by column chromatography (SiO2, PE/EtOAc=2/1 to 2/1) to give the title compound (2 batches, 5.5 g, 36.94% yield) as white solid. 1H NMR (400 MHz, 6d-DMSO): δ ppm 13.50-13.29 (m, 1H), 8.07-7.91 (m, 1H), 7.54-7.37 (m, 1H), 2.42-2.38 (m, 3H)

Step 3: Synthesis of 4-bromo-5-fluoro-6-methyl-2-(tetrahydro-2H-pyran-2-yl)-2H-indazole

A mixture of the title compound from step 2 (5.5 g, 24.01 mmol), PPTS (384.0 mg, 1.53 mmol) and DHP (3.86 g, 45.84 mmol) in DCM (45 mL) was stirred at 30° C. for 2 h. The reaction mixture was diluted with brine (20 mL) and extracted with EtOAc (3×20 mL). The combined organic layers were washed with brine (3×10 mL), dried over Na₂SO₄, filtered and concentrated under reduced pressure to give an oil. The oil was purified by column chromatography (SiO2, PE/EtOAc=10/1 to 1/1) to give the title compound (5.5 g, 73.08% yield) white solid. 1H NMR (400 MHz, 6d-DMSO): δ ppm 8.51-8.43 (m, 1H), 7.66-7.53 (m, 1H), 5.77-5.69 (m, 1H), 4.01-3.95 (m, 1H), 3.75-3.67 (m, 1H), 2.41-2.33 (m, 3H), 2.26-2.16 (m, 1H), 2.07-2.00 (m, 1H), 1.98-1.91 (m, 1H), 1.76-1.68 (m, 1H), 1.63-1.57 (m, 2H)

Step 4. 4-bromo-3,5-difluoro-6-methyl-2-(tetrahydro-2H-pyran-2-yl)-2H-indazole

Solution 1: title compound from step 3 (5.5 g, 17.56 mmol) in THF, 220 mL

Solution 2: LDA (1 M, 43.91 mL)

Solution 3: NFSI (8.31 g, 26.34 mmol) in THF, 220 mL.

Solution 1 was pumped by Pump 1 S1, P1, 2.751 mL/min to flow reactor 1 FLR1, PFA, Coils reactor, 1.590( 1/16″) mm, 0.636 mL, 25° C.

Solution 2 was pumped by Pump 2 S2, P2, 0.429 mL/min to flow reactor 1 FLR1, PFA, Coils reactor, 1.590( 1/16″) mm, 0.636 mL, 25° C.

Solution 3 was pumped by Pump 3 S3, P3, 2.781 mL/min to flow reactor2 FLR2, PFA, Coils reactor, 1.590( 1/16″) mm, 0.636 mL, 60° C.

The residence time of flow reactor 1 was FLR1, 0.2 min.

The residence time of flow reactor 2 was FLR2, 0.104 min.

The mixture was quenched by slowly adding to 300 mL saturated aqueous NH₄Cl, extracted with EtOAc (3×200 mL). The combined organic layers were washed with brine (3×50 mL), dried over Na₂SO₄, filtered and concentrated under reduced pressure. The residue was purified by column chromatography (SiO₂, PE/EtOAc=20/1 to 1/1) to give the title compound (1.7 g, 22.21% yield) as a white solid. LCMS: m/z=331 [M+H]⁺

Step 5: Synthesis of tert-butyl (3-cyano-4-(3,5-difluoro-6-methyl-2H-indazol-4-yl)-7-fluorobenzo[b]thiophen-2-yl)carbamate

A mixture of Intermediate 9 (976.61 mg, 2.42 mmol), the title compound from step 4 (800 mg, 2.42 mmol), Pd(dppf)Cl₂ (176.76 mg, 241.58 μmol) and Cs₂CO₃ (1.97 g, 6.04 mmol) in dioxane (15 mL) was stirred at 100° C. for 1 h under N₂. The reaction mixture was diluted with water (10 mL) and extracted with EtOAc (3×20 mL). The combined organic layers were washed with brine, dried over Na₂SO₄, filtered and concentrated under reduced pressure. The crude product was purified by reversed-phase flash chromatography ([water (FA)-ACN]; gradient:20%-100% B, 15 min). To the obtained solution was added FA (5 mL), then lyophilized to give the compound the title compound (930 mg, 39.05% yield) as white solid. LCMS: m/z=459.1 [M+H]⁺

Step 6: Synthesis of tert-butyl (3-cyano-4-(3,5-difluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophen-2-yl)carbamate

A mixture of Intermediate 1 (64.95 mg, 218.12 μmol), the title compound from Step 5 (100 mg, 218.12 μmol), EPhos Pd G4 (20.04 mg, 21.81 μmol) and Cs₂CO₃ (177.67 mg, 545.31 μmol) in dioxane (1 mL) was degassed and purged with N₂ for 3 times, and then the mixture was stirred at 100° C. for 1 h under N₂. The reaction mixture was diluted with water (10 mL) and extracted with EtOAc (3×20 mL). The combined organic layers were washed with brine (3×10 mL), dried over Na₂SO₄, filtered and concentrated under reduced pressure to give the title compound (200 mg, crude) as white solid. LCMS: m/z=720.3 [M+H]⁺

Step 7: Synthesis of (S)-2-amino-4-(3,5-difluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile and (R)-2-amino-4-(3,5-difluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

A mixture of the title compound from step 6 (100 mg, 138.93 μmol) and TFA (1.54 g, 13.46 mmol) in DCM (2 mL) was stirred at 25° C. for 10 min. The mixture was concentrated then purified by reversed-phase HPLC (column: Phenomenex Luna C18 150*25 mm*10 um; mobile phase: [H2O(0.1% TFA)-ACN]; gradient:20%-50% B over 15.0 min) to give the compound Peak 1, Example 152, (S)-2-amino-4-(3,5-difluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (R)-2-amino-4-(3,5-difluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile (22.8 mg, 26.48% yield, 98.9% ee) as yellow solid and Peak 2, Example 153, (R)-2-amino-4-(3,5-difluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (S)-2-amino-4-(3,5-difluoro-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile (24.6 mg, 28.44% yield, 97.4% ee) as yellow solid.

Peak 1, Example 152, LCMS: m/z=620.2 [M+H]⁺, 1H NMR (400 MHz, 6d-DMSO): δ ppm 9.97-9.82 (m, 1H), 8.71-8.62 (m, 1H), 8.15-8.03 (m, 2H), 7.43-7.33 (m, 1H), 7.22-7.12 (m, 1H), 5.47-5.35 (m, 1H), 4.73-4.58 (m, 2H), 4.06 (br s, 2H), 3.83-3.77 (m, 1H), 3.62-3.56 (m, 1H), 3.22-3.15 (m, 1H), 3.05-2.91 (m, 5H), 2.52-2.51 (m, 3H), 2.33-2.25 (m, 1H), 2.14-2.04 (m, 1H), 2.03-1.85 (m, 2H), 1.57-1.45 (m, 3H)

Peak 2, Example 153, LCMS: m/z=620.2 [M+H]⁺, 1H NMR (400 MHz, 6d-DMSO): δ ppm 10.08-9.91 (m, 1H), 8.71-8.60 (m, 1H), 8.21-7.99 (m, 2H), 7.45-7.34 (m, 1H), 7.22-7.11 (m, 1H), 5.47-5.32 (m, 1H), 4.56 (s, 1H), 4.08-3.98 (m, 2H), 3.83-3.75 (m, 1H), 3.61-3.53 (m, 1H), 3.22-3.12 (m, 1H), 3.08-2.98 (m, 2H), 2.97-2.85 (m, 3H), 2.52-2.50 (m, 3H), 2.34-2.24 (m, 1H), 2.13-2.04 (m, 1H), 2.02-1.86 (m, 2H), 1.58-1.44 (m, 3H)

### Example 154

### (R)-2-amino-7-fluoro-4-(3-fluoro-5,6-dimethyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophene-3-carbonitrile or (S)-2-amino-7-fluoro-4-(3-fluoro-5,6-dimethyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophene-3-carbonitrile

### And Example 155

### (S)-2-amino-7-fluoro-4-(3-fluoro-5,6-dimethyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophene-3-carbonitrile or (R)-2-amino-7-fluoro-4-(3-fluoro-5,6-dimethyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophene-3-carbonitrile

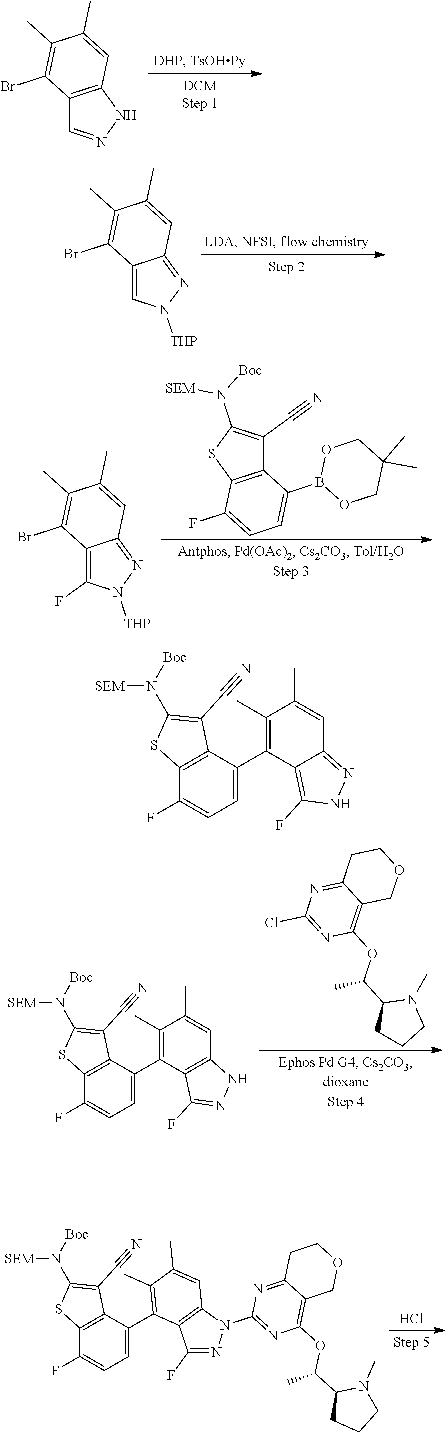

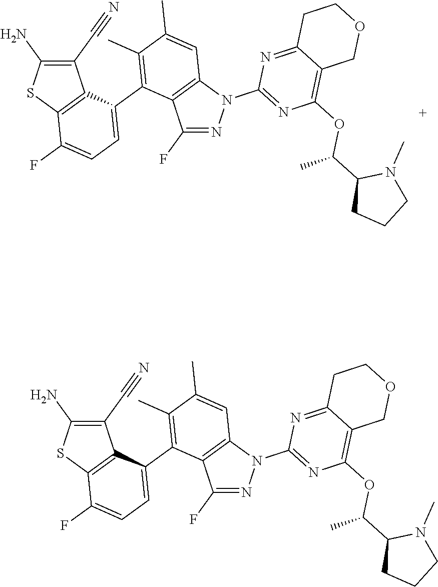

Step 1: Synthesis of 4-bromo-5,6-dimethyl-2-(tetrahydro-2H-pyran-2-yl)-2H-indazole

To a solution of 4-bromo-5,6-dimethyl-1H-indazole (2.80 g, 12.4 mmol) in DCM (30 mL) was added pyridinium p-toluenesulfonate (312 mg, 1.24 mmol) and 3,4-dihydro-2H-pyran (3.14 g, 37.3 mmol) at 25° C. and the mixture was stirred for 12 h. The mixture was cooled to 20° C., then quenched by sat'd aqueous NaHCO₃ solution slowly until pH=7~8. The mixture was stirred at 20° C. for 20 min then diluted with water (20 mL), extracted with DCM (10 mL). The organic layers were concentrated under reduced pressure. The residue was purified by column chromatography (SiO2, PE/EtOAc=40/1 to 1/1) to give title compound (800 mg, 21% yield) as a yellow solid. 1H NMR (400 MHz, CDCl₃) δ=8.06 (s, 1H), 7.43 (s, 1H), 5.72-5.58 (m, 1H), 4.24-4.09 (m, 1H), 3.93-3.66 (m, 1H), 2.42 (d, 6H), 2.25-2.17 (m, 2H), 2.08-2.01 (m, 1H), 1.80-1.62 (m, 3H).

Step 2: Synthesis of 4-bromo-3-fluoro-5,6-dimethyl-2-(tetrahydro-2H-pyran-2-yl)-2H-indazole

Solution 1: title compound from step 1 (700 mg, 2.26 mmol) in THF (20 mL).

Solution 2: LDA (2 M, 1.92 mL).

Solution 3: NSFI (1.21 g, 3.85 mmol) in THF (22 mL).

Solution 1 was pumped by Pump 1 S1, P1, 2.904 mL/min to flow reactor 1 FLR1, PFA, Coils reactor, 1.588( 1/16″) mm, 0.636 mL, 0° C.

Solution 2 was pumped by Pump 2 S2, P2, 0.276 mL/min to flow reactor 1 FLR1, PFA, Coils reactor, 1.588( 1/16″) mm, 0.636 mL, 0° C.

Solution 3 was pumped by Pump 3 S3, P3, 2.938 mL/min to flow reactor 2 FLR2, PFA, Coils reactor, 1.590( 1/16″) mm, 0.636 mL, 20° C.

The residence time of flow reactor 1 was FLR1, 0.2 min. The residence time of flow reactor 2 was FLR2, 0.104 min. The mixture was quenched by water (30 mL) at 0° C. and then diluted with water (10 mL) and extracted with EtOAc (2×50 mL). The combined organic layers were washed with brine (50 mL), dried over Na₂SO₄, filtered and concentrated under reduced pressure. The residue was purified by column chromatography (SiO2, PE/EtOAc=40/1 to 3/1) to give the title compound (440 mg, 56% yield) as a yellow solid. 1H NMR (400 MHz, CDCl₃) δ=7.26 (s, 1H), 5.73-5.55 (m, 1H), 4.20-4.06 (m, 1H), 3.82-3.59 (m, 1H), 2.57-2.42 (m, 1H), 2.37 (s, 6H), 2.22-2.11 (m, 1H), 2.04-1.93 (m, 1H), 1.84-1.70 (m, 2H), 1.70-1.58 (m, 1H).

Step 3: Synthesis of 4-bromo-3-fluoro-5,6-dimethyl-2-(tetrahydro-2H-pyran-2-yl)-2H-indazole

A mixture of the title compound from step 2 (340 mg, 1.04 mmol), Intermediate 10 (1.67 g, 3.12 mmol), Cs₂CO₃ (1.02 g, 3.12 mmol), 4-(9-anthryl)-3-tert-butyl-2H-1,3-benzoxaphosphole (38.4 mg, 103 μmol) and palladium (ii)acetate (23.3 mg, 103 μmol) in toluene (10 mL) and water (2 mL) was degassed and purged with N₂ for 3 times, and then the mixture was stirred at 100° C. for 12 h under N₂. The reaction mixture was partitioned between EtOAc (20 mL) and water (5 mL). The organic phase was separated, washed with brine (5 mL×2), dried over with Na₂SO₄, filtered and concentrated under reduced pressure. The residue was purified by prep-HPLC (column: Phenomenex luna C18 150*25 mm*10 um; mobile phase: [H2O (0.225% FA)-ACN]; gradient:80%-100% B over 10.0 min) to give the title compound (360 mg, 59% yield) as a yellow solid. 1H NMR (400 MHz, CDCl₃) δ=9.11 (s, 1H), 7.38-7.30 (m, 1H), 7.24 (d, 2H), 5.20-5.03 (m, 2H), 3.80-3.59 (m, 2H), 2.46 (s, 3H), 2.04 (s, 3H), 1.41 (s, 9H), 1.11-0.95 (m, 2H), 0.02 (s, 9H).

Step 4: Synthesis of tert-butyl (3-cyano-7-fluoro-4-(3-fluoro-5,6-dimethyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophen-2-yl)((2-(trimethylsilyl)ethoxy)methyl)carbamate

A mixture of the title compound from Step 3 (160 mg, 273 μmol), Intermediate 1 (81.4 mg, 273 μmol), EPhos Pd G4 (25.1 mg, 27.3 μmol), Cs₂CO₃ (178 mg, 547 μmol) in dioxane (4 mL) was degassed and purged with N₂ for 3 times, and then the mixture was stirred at 100° C. for 0.5 h under N₂. The reaction mixture was filtered and concentrated under reduced pressure to give a residue. The residue was purified by prep-HPLC (column: Phenomenex Luna C18 150*25 mm*10 um; mobile phase: [H2O(0.1% TFA)-ACN]; gradient:43%-73% B over 15.0 min) to give the title compound (150 mg, 64% yield) as a yellow solid. LCMS: m/z=846 [M+H]⁺

Step 5: Synthesis of (R)-2-amino-7-fluoro-4-(3-fluoro-5,6-dimethyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophene-3-carbonitrile and (S)-2-amino-7-fluoro-4-(3-fluoro-5,6-dimethyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophene-3-carbonitrile

A solution of the title compound from step 4 (130 mg, 153 μmol) in aqueous HCl (6 M, 6.50 mL) was stirred at 40° C. for 0.5 h. The reaction mixture was concentrated under reduced pressure to give a residue. The residue was dissolved in THF (0.5 mL), then ammonium hydroxide (0.5 mL) was added, the resulting mixture was stirred at 20° C. for 0.5 h. The mixture was diluted with water (10 mL) and extracted with EtOAc (2×10 mL). The combined organic layers were washed with brine (10 mL), dried over Na₂SO₄ filtered and concentrated under reduced pressure. The residue was purified by reversed-phase HPLC (column: Phenomenex luna C18 150*25 mm*10 um; mobile phase: [H2O (0.1% TFA)-ACN]; gradient:23%-53% B over 10.0 min) to give Peak 1, Example 154, (R)-2-amino-7-fluoro-4-(3-fluoro-5,6-dimethyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophene-3-carbonitrile or (S)-2-amino-7-fluoro-4-(3-fluoro-5,6-dimethyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophene-3-carbonitrile (16.3 mg, 17% yield) as a white solid and Peak 2, Example 155, (S)-2-amino-7-fluoro-4-(3-fluoro-5,6-dimethyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophene-3-carbonitrile or (R)-2-amino-7-fluoro-4-(3-fluoro-5,6-dimethyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophene-3-carbonitrile (7.00 mg, 7% yield) as a white solid.

Peak 1, Example 154, LCMS: m/z=616.3 [M+H]⁺, 1H NMR (400 MHz, DMSO-d6) δ=9.92-9.74 (m, 1H), 8.56 (s, 1H), 7.98 (s, 2H), 7.23-7.17 (m, 1H), 7.17-7.10 (m, 1H), 5.52-5.32 (m, 1H), 4.72-4.54 (m, 2H), 4.10-3.96 (m, 2H), 3.84-3.74 (m, 1H), 3.59 (dd, 1H), 3.24-3.14 (m, 1H), 2.97 (d, 5H), 2.51-2.51 (m, 3H), 2.35-2.29 (m, 1H), 2.16-2.06 (m, 1H), 2.02 (s, 3H), 2.00-1.85 (m, 2H), 1.51 (d, 3H).

Peak 2, Example 155, LCMS: m/z=616.3 [M+H]⁺, 1H NMR (400 MHz, DMSO-d6) δ=9.86-9.71 (m, 1H), 8.55 (s, 1H), 7.98 (s, 2H), 7.25-7.18 (m, 1H), 7.18-7.10 (m, 1H), 5.48-5.33 (m, 1H), 4.73-4.56 (m, 2H), 4.09-3.96 (m, 2H), 3.85-3.71 (m, 1H), 3.57-3.52 (m, 1H), 3.22-3.14 (m, 1H), 3.04-2.89 (m, 5H), 2.52-2.51 (m, 3H), 2.33-2.26 (m, 1H), 2.15-2.05 (m, 1H), 2.02 (s, 3H), 1.99-1.86 (m, 2H), 1.53-1.45 (m, 3H).

### Example 156

### (S)-2-amino-7-fluoro-4-(3-fluoro-5-methoxy-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophene-3-carbonitrile or (R)-2-amino-7-fluoro-4-(3-fluoro-5-methoxy-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophene-3-carbonitrile

### And Example 157

### (R)-2-amino-7-fluoro-4-(3-fluoro-5-methoxy-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophene-3-carbonitrile(S)-2-amino-7-fluoro-4-(3-fluoro-5-methoxy-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophene-3-carbonitrile

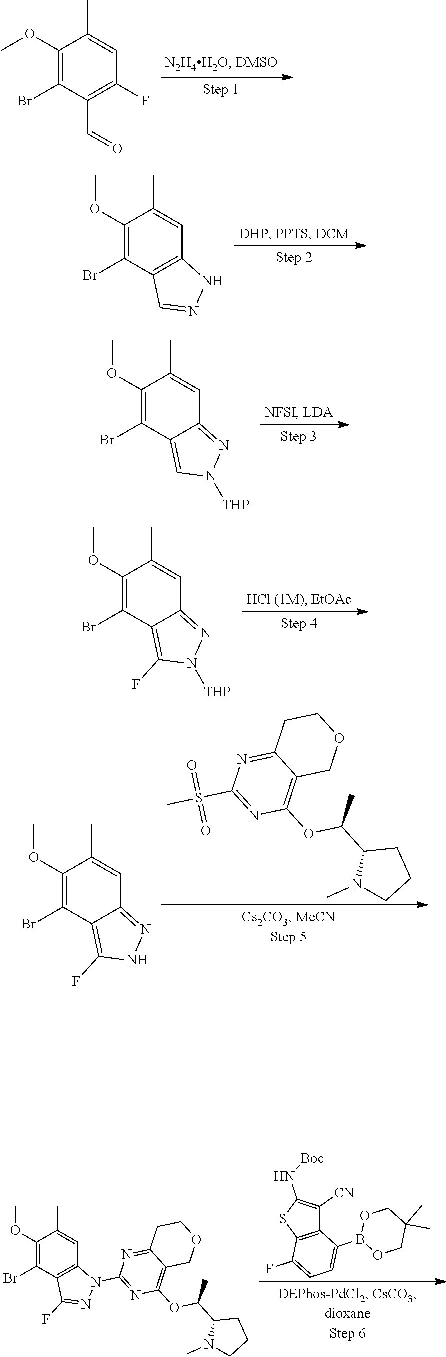

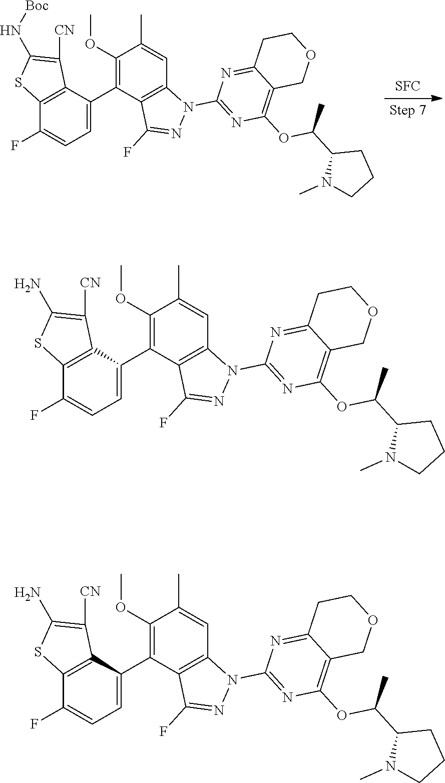

Step 1: Synthesis of 4-bromo-5-methoxy-6-methyl-1H-indazole

To a solution of 2-bromo-6-fluoro-3-methoxy-4-methylbenzaldehyde (1.5 g, 6.07 mmol) in DMSO (15 mL) was added hydrazine hydrate (3.20 g, 60.7 mmol, 95% purity), then the reaction mixture was stirred at 135° C. for 16 h. The mixture was cooled to 20° C., then 1 N aqueous HCl solution was added slowly adjusting to pH 6, stirred at 20° C. for 20 min, and extracted with EtOAc (2×40 mL). The combined organic layers were dried over Na₂SO₄, filtered and concentrated under reduced pressure. The residue was purified by column chromatography (silica gel, hexanes: EtOAc=5:1) to give the title compound (330 mg, 1.37 mmol, 22% yield) as a yellow solid. 1H NMR (400 MHz, CDCl₃) δ=8.04 (s, 1H), 7.29 (s, 1H), 3.89 (s, 3H), 2.49 (s, 3H).

Step 2: Synthesis of 4-bromo-5-methoxy-6-methyl-2-(tetrahydro-2H-pyran-2-yl)-2H-indazole

To a solution of the title compound from step 1 (280 mg, 1.16 mmol, 1 equiv) and 3,4-dihydro-2H-pyran (293 mg, 3.48 mmol) in DCM (6 mL) was added PPTS (29.1 mg, 116 μmol) and the mixture was stirred at 30° C. for 1.5 h. The reaction was washed with water and extracted with EtOAc (2×40 mL) dried over Na₂SO₄, filtered and concentrated under reduced pressure. The residue was purified by column chromatography (silica gel, hexanes: EtOAc=10:1) to give the title compound as a brown oil. ¹H NMR (400 MHz, CDCl₃) δ=8.09 (s, 1H), 7.45 (s, 1H), 5.69-5.58 (m, 1H), 4.20-4.08 (m, 1H), 3.86 (s, 3H), 3.78 (dt, 1H), 2.43 (s, 3H), 2.26-2.16 (m, 2H), 2.12-2.00 (m, 1H), 1.83-1.66 (m, 3H)

Step 3: Synthesis of 4-bromo-3-fluoro-5-methoxy-6-methyl-2-(tetrahydro-2H-pyran-2-yl)-2H-indazole

Solution 1: The title compound from step 3 (370 mg, 1.14 mmol) in THF (18.5 mL)

Solution 2: LDA (1 M, 1.37 mL)

Solution 3: NSFI (538.17 mg, 1.71 mmol) in THF (18.5 mL).

The solution1 was pumped by Pump 1 {S1, P1, 0.306 mL/min} to flow reactor 1 {FLR1, PFA, Coils reactor, 1.588( 1/16″) mm, 0.098 mL, 0.0° C.}. The solution 2 was pumped by Pump 2 {S2, P2, 0.086 mL/min} to flow reactor1 {FLR1, PFA, Coils reactor, 1.588( 1/16″) mm, 0.098 mL,0.0° C.}. The solution 3 was pumped by Pump 3 {S3, P3, 0.315 mL/min} to flow reactor 2 {FLR2, PFA, Coils reactor, 1.588( 1/16″) mm, 0.098 mL, 20.0° C.}. The residence time of flow reactor 1 {FLR1, 0.25 min}. The residence time of flow reactor 2 {FLR2, 0.139 min}. The mixture was collected with a bottle and quenched by 20 mL water at 0° C. The mixture was collected with a bottle and quenched by 20 mL brine at 0° C. The mixture was separated. The organic phase was washed with 20 mL of brine, dried over Na₂SO₄ and concentrated. The residue was purified by column chromatography (silica gel, hexanes: EtOAc=50:1 to 10:1) to give the title compound (30 mg, 87.4 μmol, 7% yield) as a brown oil. LCMS: m/z=343.0 [M+H]⁺.

Step 4: Synthesis of 4-bromo-3-fluoro-5-methoxy-6-methyl-2H-indazole

To a solution of the title compound from step 3 (30 mg, 87.4 μmol) in EtOAc (0.5 mL) was added HCl (1 M aqueous). The mixture was stirred at 20° C. for 0.5 h then quenched by NaHCO₃ until pH=8 at 15° C. and then extracted with EtOAc (2×10 mL). The combined organic layers were dried over Na₂SO₄, filtered and concentrated under reduced pressure. The residue was purified by column chromatography (silica gel, hexanes: EtOAc=10:1 to 3:1) to give the title compound (20 mg, crude) as a white solid. LCMS: m/z=259.0 [M+H]⁺.

Step 5: Synthesis of 2-(4-bromo-3-fluoro-5-methoxy-6-methyl-1H-indazol-1-yl)-4-((S)-1-((S)-1-methylpyrrolidin-2-yl)ethoxy)-7,8-dihydro-5H-pyrano[4,3-d]pyrimidine

To a mixture of the title compound from Step 4 (60 mg, 231 μmol) and title compound from Intermediate 39, step 1 (94.8 mg, 277 μmol) in acetonitrile (1 mL) was added Cs₂CO₃ (150 mg, 463 μmol) and the mixture was stirred at 25° C. for 2 h. The reaction was filtered. The filtrate was purified by Prep-HPLC column: Phenomenex Luna C18 150*25 mm*10 um; mobile phase: [H2O(0.225% FA)-ACN]; gradient:20%-50% B over 15.0 min to give the title compound (80 mg, 152 μmol, 65% yield) as a white solid. ¹H NMR (400 MHz, CDCl₃) δ=8.46 (s, 1H), 5.45 (quin, 1H), 4.77-4.60 (m, 2H), 4.13-3.98 (m, 2H), 3.89 (s, 3H), 3.16 (br t, 1H), 3.01 (t, 2H), 2.76-2.63 (m, 1H), 2.50 (s, 6H), 2.42-2.31 (m, 1H), 2.03-1.95 (m, 2H), 1.82 (br dd, 2H), 1.45 (d, 3H)

Step 6: Synthesis of tert-butyl (3-cyano-7-fluoro-4-(3-fluoro-5-methoxy-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophen-2-yl)carbamate

A mixture of the title compound from step 7 (80 mg, 153 μmol), Intermediate 9 (124 mg, 307 μmol), DPEPhos-PdCl₂ (22.0 mg, 30.7 μmol) and Cs₂CO₃ (150 mg, 461 μmol) in dioxane (2 mL) was degassed and purged with N₂ for 3 times and then stirred at 100° C. for 16 h under N₂. The reaction was then filtered, and the filtrate was concentrated. The residue was purified by Prep-HPLC (column: Phenomenex Luna C18 150*25 mm*10 um; mobile phase:[H2O(0.1% TFA)-ACN]; gradient:25%-55% B over 15.0 min) to give the title compound (45 mg, 49.1 μmol, 32% yield) as a yellow solid. LCMS: m/z=732.3 [M+H]⁺.

Step 7: Synthesis of (S)-2-amino-7-fluoro-4-(3-fluoro-5-methoxy-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophene-3-carbonitrile and (R)-2-amino-7-fluoro-4-(3-fluoro-5-methoxy-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophene-3-carbonitrile

To a solution of the title compound from step 6 (40 mg, 54.6 μmol) in DCM (1 mL) was added TFA (767 mg, 6.73 mmol) and the mixture was stirred at 20° C. for 1 h then concentrated. The residue was purified by Prep-HPLC (column: Phenomenex Luna C18 150*25 mm*10 um; mobile phase: [H2O(0.1% TFA)-ACN]; gradient:18%-48% B over 15.0 min) and SFC (column: DAICEL CHIRALPAK AD (250 mm*30 mm, 10 um); mobile phase: [CO2-EtOH (0.1% NH3H2O)]; B %: 40%, isocratic elution mode) to give Peak 1, Example 156, (S)-2-amino-7-fluoro-4-(3-fluoro-5-methoxy-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophene-3-carbonitrile or (R)-2-amino-7-fluoro-4-(3-fluoro-5-methoxy-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophene-3-carbonitrile (13.5 mg, 37% yield, TFA, 94.7% ee) and Peak 2, Example 157, (R)-2-amino-7-fluoro-4-(3-fluoro-5-methoxy-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophene-3-carbonitrile or (S)-2-amino-7-fluoro-4-(3-fluoro-5-methoxy-6-methyl-1-(4-((S)-1-((S)-1-methylpyrrolidin-2-yl)ethoxy)-7,8-dihydro-5H-pyrano[4,3-d]pyrimidin-2-yl)-1H-indazol-4-yl)benzo[b]thiophene-3-carbonitrile, (12 mg, 39% yield, 99.1% ee) as an off-white solid.

Peak 1, Example 156, LCMS: m/z=732.3 [M+H]⁺, ¹H NMR (DMSO-d6, 400 MHz) δ=9.92 (br s, 1H), 8.59 (s, 1H), 7.98 (s, 2H), 7.33 (dd, 1H), 7.14 (t, 1H), 5.41 (dd, 1H), 4.80-4.54 (m, 2H), 4.06-3.94 (m, 2H), 3.79 (br dd, 1H), 3.65-3.54 (m, 1H), 3.37 (s, 3H), 3.19 (br dd, 1H), 2.97 (br d, 5H), 2.49 (br s, 3H), 2.38-2.24 (m, 1H), 2.16-2.03 (m, 1H), 2.01-1.87 (m, 2H), 1.51 (d, 3H)

Peak 2, Example 157, LCMS: m/z=732.3 [M+H]⁺, ¹H NMR (DMSO-d6, 400 MHz) δ=8.58 (s, 1H), 7.99 (s, 2H), 7.33 (dd, 1H), 7.13 (t, 1H), 5.52-5.40 (m, 1H), 4.63 (br d, 2H), 4.01 (br s, 2H), 3.50-3.38 (m, 2H), 3.36 (br s, 3H), 2.94 (br s, 3H), 2.83-2.66 (m, 2H), 2.54-2.51 (m, 1H), 2.48 (s, 3H), 2.26-1.94 (m, 2H), 1.85 (br d, 2H), 1.46 (br d, 3H)

### Example 158

### (S)-2-amino-4-(5-chloro-3-fluoro-6-(methylamino)-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (R)-2-amino-4-(5-chloro-3-fluoro-6-(methylamino)-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

### And Example 159

### (R)-2-amino-4-(5-chloro-3-fluoro-6-(methylamino)-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (S)-2-amino-4-(5-chloro-3-fluoro-6-(methylamino)-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

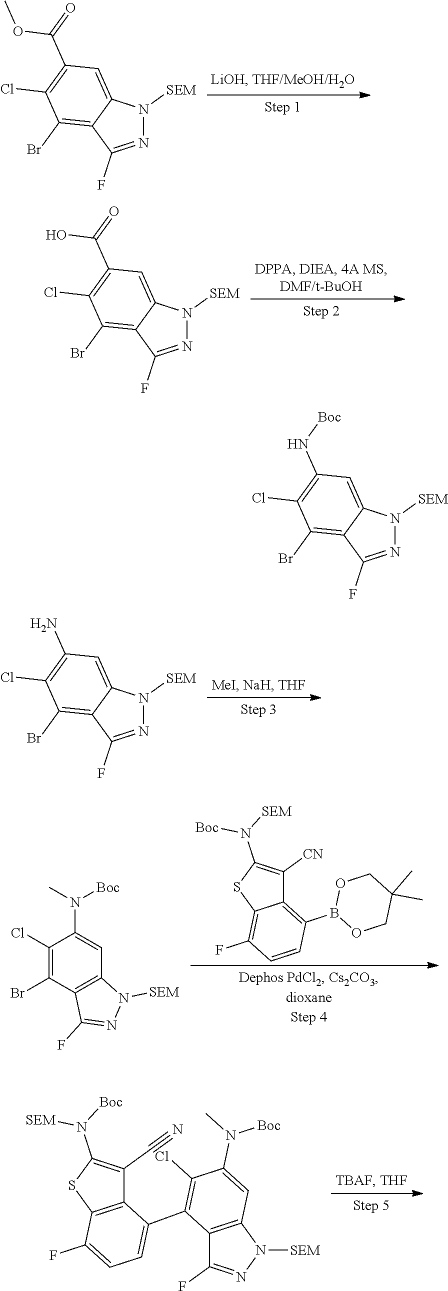

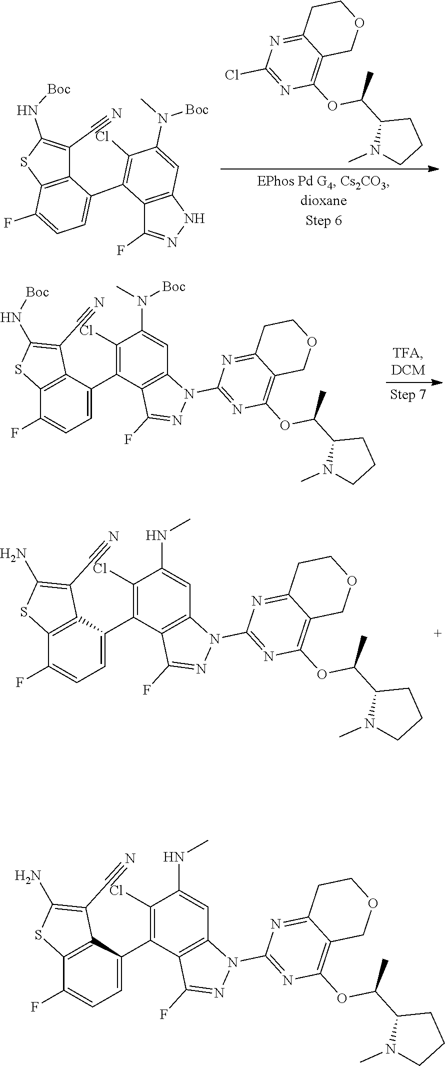

Step 1: Synthesis of 4-bromo-5-chloro-3-fluoro-1-((2-(trimethylsilyl)ethoxy)methyl)-1H-indazole-6-carboxylic acid

A mixture of the title compound from Intermediate 38, step 1 (550 mg, 1.22 mmol), LiOH·H2O(510.81 mg, 12.17 mmol) in THF (5 mL), H2O (5 mL) and MeOH (5 mL) was stirred at 40° C. for 1 h. To the mixture was added 1.2 mL 1 N HCl to adjust pH to 4, then the mixture was diluted with water (10 mL) and extracted with EtOAc (3×20 mL). The combined organic layers were washed with water (3×10 mL), dried over Na₂SO₄, filtered and concentrated under reduced pressure to give the title compound (510 mg, 98.87% yield) as a white solid. 1H NMR (400 MHz, 6d-DMSO) δ 8.04 (s, 1H), 5.67 (s, 2H), 3.53-3.50 (m, 2H), 0.80 (t, 2H), −0.09 (s, 8H).

Step 2: Synthesis of tert-butyl (4-bromo-5-chloro-3-fluoro-1-((2-(trimethylsilyl)ethoxy)methyl)-1H-indazol-6-yl)carbamate and 4-bromo-5-chloro-3-fluoro-1-((2-(trimethylsilyl)ethoxy)methyl)-1H-indazol-6-amine

A mixture of the title compound from step 1 (500 mg, 1.18 mmol), DPPA (649.46 mg, 2.36 mmol), DIEA (457.51 mg, 3.54 mmol), t-BuOH (3.87 g, 52.28 mmol, 5.00 mL) and 4A MS (1 g) in DMF (10 mL) was degassed and purged with N₂ 3 times, and then the mixture was stirred at 90° C. for 1 h under N₂. The reaction mixture was diluted with water (20 mL) and extracted with EtOAc (3×20 mL). The combined organic layers were washed with brine, dried over Na₂SO₄, filtered and concentrated under reduced pressure. The residue was purified by reversed-phase flash chromatography ([water (FA)-ACN]; gradient:30%-90% B, 15 min) to give the compounds tert-butyl (4-bromo-5-chloro-3-fluoro-1-((2-(trimethylsilyl)ethoxy)methyl)-1H-indazol-6-yl)carbamate (520 mg, 86.50% yield) LCMS: m/z=494.1 [M+H]⁺ and

4-bromo-5-chloro-3-fluoro-1-((2-(trimethylsilyl)ethoxy)methyl)-1H-indazol-6-amine (330 mg, 64.14% yield). LCMS: m/z=394.1 [M+H]⁺

Step 3: Synthesis of tert-butyl (4-bromo-5-chloro-3-fluoro-1-((2-(trimethylsilyl)ethoxy)methyl)-1H-indazol-6-yl) (methyl)carbamate

To a mixture of tert-butyl (4-bromo-5-chloro-3-fluoro-1-((2-(trimethylsilyl)ethoxy)methyl)-1H-indazol-6-yl)carbamate (280 mg, 565.82 μmol) in THF (10 mL) was added NaH (33.95 mg, 848.73 μmol, 60% in oil) at 0° C. under N₂. MeI (160.62 mg, 1.13 mmol, 70.45 μL) was added and the mixture was stirred at 25° C. for 1 h under N₂. The mixture was quenched with water (0.2 mL), then diluted with water (10 mL) and extracted with EtOAc (3×20 mL). The combined organic layers were washed with brine (3×10 mL), dried over Na₂SO₄ filtered and concentrated under reduced pressure. The residue was purified by column chromatography (SiO2, PE/EtOAc-50/1 to 5/1) to give compound the title compound (two batches, 340 mg, 55.77% yield) as white solid. LCMS: m/z=508.1 [M+H]⁺

Step 4: tert-butyl (4-(6-((tert-butoxycarbonyl)(methyl)amino)-5-chloro-3-fluoro-1-((2-(trimethylsilyl)ethoxy)methyl)-1H-indazol-4-yl)-3-cyano-7-fluorobenzo[b]thiophen-2-yl)((2-(trimethylsilyl)ethoxy)methyl)carbamate

A mixture of the title compound from step 3 (535.69 mg, 1.00 mmol), Intermediate 10 (340 mg, 668.13 μmol), DPEphos PdCl₂ (47.83 mg, 66.81 μmol), and Cs₂CO₃ (544.22 mg, 1.67 mmol) in dioxane (15 mL) was stirred at 80° C. for 1 h under N₂. The reaction mixture was diluted with water (10 mL) and extracted with EtOAc (3×20 mL). The combined organic layers were washed with brine (3×10 mL), dried over Na₂SO₄ filtered and concentrated under reduced pressure. The residue was purified by column chromatography (SiO2, PE/EtOAc=10:1) to give the compound the title compound (367 mg, 62.28% yield) as white solid. LCMS: m/z=850.3 [M+H]⁺

Step 5: tert-butyl (4-(2-((tert-butoxycarbonyl)amino)-3-cyano-7-fluorobenzo[b]thiophen-4-yl)-5-chloro-3-fluoro-1H-indazol-6-yl)(methyl)carbamate

A mixture of the title compound from step 4 (367 mg, 431.48 μmol) in TBAF (1 M, 25 mL) was stirred at 70° C. for 16 h then concentrated under reduced pressure. The residue was purified by column chromatography (SiO2, PE/EtOAc=10/1 to 3/1) to give the title compound (130 mg, 50.22% yield) as a white solid. LCMS: m/z=590.1 [M+H]⁺

Step 6: Synthesis of tert-butyl (4-(2-((tert-butoxycarbonyl)amino)-3-cyano-7-fluorobenzo[b]thiophen-4-yl)-5-chloro-3-fluoro-1-(4-((S)-1-((S)-1-methylpyrrolidin-2-yl)ethoxy)-7,8-dihydro-5H-pyrano[4,3-d]pyrimidin-2-yl)-1H-indazol-6-yl)(methyl)carbamate

A mixture of the title compound from step 5 (100 mg, 169.48 μmol), Intermediate 1 (50.47 mg, 169.48 μmol), Cs₂CO₃ (138.05 mg, 423.70 μmol), and EPhos Pd G4 (15.57 mg, 16.95 μmol) in dioxane (10 mL) was degassed and purged with N₂ for 3 times, then stirred at 100° C. for 1 h under N₂. The mixture was filtered and concentrated to give the title compound (crude, 130 mg, 90.10% yield) as a yellow solid.

Step 7: Synthesis of (S)-2-amino-4-(5-chloro-3-fluoro-6-(methylamino)-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile and (R)-2-amino-4-(5-chloro-3-fluoro-6-(methylamino)-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile

A mixture of the title compound from step 6 (125 mg, 146.82 μmol) and TFA (6.14 g, 53.85 mmol, 4 mL) in DCM (10 mL) was stirred at 25° C. for 10 min. The mixture was concentrated to give a residue, which was purified by reversed-phase HPLC (column: Phenomenex Luna C18 150*25 mm*10 um; mobile phase: [H2O(0.225% FA)-ACN]; gradient:18%-48% B over 15.0 min) to give compound Peak 1, Example 158, (S)-2-amino-4-(5-chloro-3-fluoro-6-(methylamino)-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (R)-2-amino-4-(5-chloro-3-fluoro-6-(methylamino)-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile (16.1 mg, 16.72% yield, 98.6% ee) and Peak 2, Example 159, (R)-2-amino-4-(5-chloro-3-fluoro-6-(methylamino)-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile or (S)-2-amino-4-(5-chloro-3-fluoro-6-(methylamino)-1-(4-((S)-1-((S)-1-methylpyrrolidin-2-yl)ethoxy)-7,8-dihydro-5H-pyrano[4,3-d]pyrimidin-2-yl)-1H-indazol-4-yl)-7-fluorobenzo[b]thiophene-3-carbonitrile (20.2 mg, 20.97% yield, 98.5% ee) as a white solid.

Peak 1, Example 158, LCMS: m/z=651.2 [M+H]⁺ 1H NMR (400 MHz, 6d-DMSO) δ 8.33-8.25 (m, 1H), 8.09-7.99 (m, 2H), 7.83-7.78 (m, 1H), 7.31-7.19 (m, 1H), 7.17-7.10 (m, 1H), 6.48-6.39 (m, 1H), 5.42-5.31 (m, 1H), 4.65-4.48 (m, 2H), 4.02-3.95 (m, 2H), 3.00-2.95 (m, 1H), 2.95-2.91 (m, 3H), 2.90-2.84 (m, 2H), 2.66-2.58 (m, 1H), 2.40-2.34 (m, 3H), 2.27-2.20 (m, 1H), 1.96-1.85 (m, 1H), 1.73-1.62 (m, 3H), 1.39-1.32 (m, 3H).

Peak 2, Example 159, LCMS: m/z=651.2 [M+H]⁺ 1H NMR (400 MHz, 6d-DMSO) δ 8.23 (s, 1H), 8.08-7.97 (m, 2H), 7.84-7.75 (m, 1H), 7.27-7.20 (m, 1H), 7.16-7.06 (m, 1H), 6.47-6.36 (m, 1H), 5.43-5.33 (m, 1H), 4.65-4.50 (m, 2H), 4.05-3.92 (m, 2H), 2.99-2.94 (m, 1H), 2.94-2.90 (m, 3H), 2.88-2.80 (m, 2H), 2.65-2.57 (m, 1H), 2.36-2.33 (m, 3H), 2.26-2.19 (m, 1H), 1.94-1.84 (m, 1H), 1.73-1.61 (m, 3H), 1.38-1.31 (m, 3H).

### Example 160

### (S)-2-amino-4-(5-chloro-3-fluoro-1-(4-((S)-1-((S)-1-methylpyrrolidin-2-yl)ethoxy)-7,8-dihydro-5H-pyrano[4,3-d]pyrimidin-2-yl)-6-(trifluoromethyl)-1H-indazol-4-yl)-7-fluorobenzo[b]thiophene-3-carbonitrile or (R)-2-amino-4-(5-chloro-3-fluoro-1-(4-((S)-1-((S)-1-methylpyrrolidin-2-yl)ethoxy)-7,8-dihydro-5H-pyrano[4,3-d]pyrimidin-2-yl)-6-(trifluoromethyl)-1H-indazol-4-yl)-7-fluorobenzo[b]thiophene-3-carbonitrile

### And Example 161

### (R)-2-amino-4-(5-chloro-3-fluoro-1-(4-((S)-1-((S)-1-methylpyrrolidin-2-yl)ethoxy)-7,8-dihydro-5H-pyrano[4,3-d]pyrimidin-2-yl)-6-(trifluoromethyl)-1H-indazol-4-yl)-7-fluorobenzo[b]thiophene-3-carbonitrile or (S)-2-amino-4-(5-chloro-3-fluoro-1-(4-((S)-1-((S)-1-methylpyrrolidin-2-yl)ethoxy)-7,8-dihydro-5H-pyrano[4,3-d]pyrimidin-2-yl)-6-(trifluoromethyl)-1H-indazol-4-yl)-7-fluorobenzo[b]thiophene-3-carbonitrile

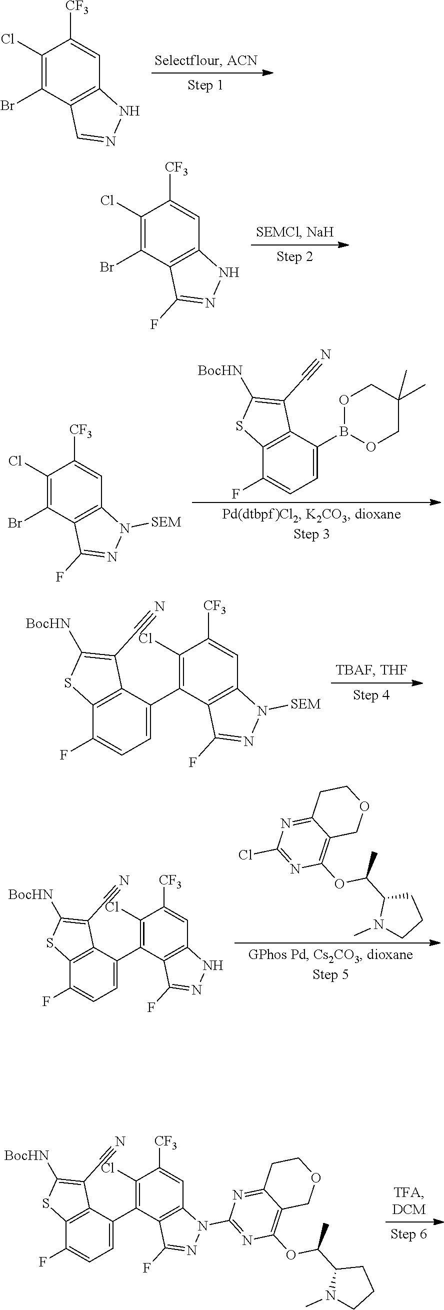

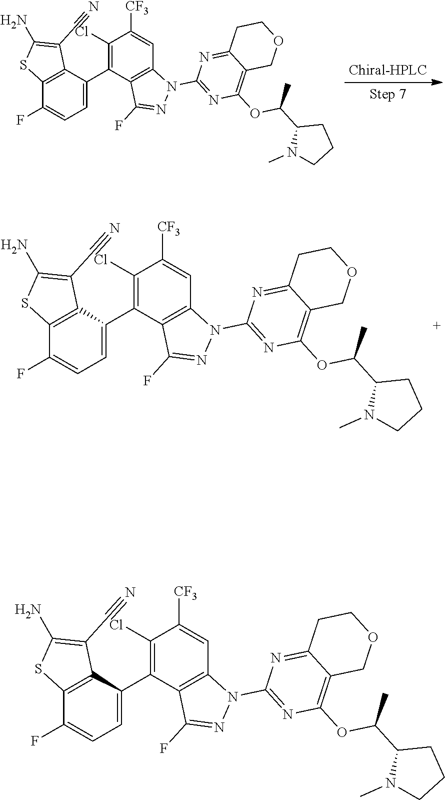

Step 1: Synthesis of 4-bromo-5-chloro-3-fluoro-6-(trifluoromethyl)-1H-indazole

To a stirred solution of 4-bromo-5-chloro-6-(trifluoromethyl)-1H-indazole (1.0 g, 3.33 mmol) in ACN (10 mL) was added Selectfluor (1.76 g, 4.99 mmol). The mixture was stirred for 12 h at 80° C. and concentrated. Water (50 mL) was added, and the mixture was extracted with DCM (3×50 mL). The combined organic layers were dried over Na₂SO₄ and concentrated. The crude product was purified by silica gel column with PE:EtOAc=3:1 to provide the title compound (330 mg, yield: 31%) as a yellow solid. LCMS: m/z=317 [M−H]

Step 2: Synthesis of 4-bromo-5-chloro-3-fluoro-6-(trifluoromethyl)-1-((2-(trimethylsilyl)ethoxy)methyl)-1H-indazole

To a stirred solution of the title compound from step 1 (300 mg, 0.944 mmol) and SEM-Cl (235 mg, 1.41 mmol) in DCM (5 mL) was added NaH (56.3 mg, 1.41 mmol) at 0° C. The mixture was stirred for 2 h at rt. Water (20 mL) was added and the mixture was extracted with DCM (3×20 mL). The combined organic layers were dried over Na₂SO₄ and concentrated. The crude product was purified by Prep-TLC with PE:EA=3:1 to afford the title compound (220 mg, yield: 52%) as a yellow oil. ¹H NMR (400 MHz, CDCl₃) δ 7.90-7.85 (m, 1H), 5.59 (s, 2H), 3.58-3.50 (m, 2H), 0.97-0.87 (m, 2H), 0.05 (s, 9H).

Step 3: Synthesis of tert-butyl (4-(5-chloro-3-fluoro-6-(trifluoromethyl)-1-((2-(trimethylsilyl)ethoxy)methyl)-1H-indazol-4-yl)-3-cyano-7-fluorobenzo[b]thiophen-2-yl)carbamate

To a stirred solution of the title compound from step 2 (220 mg, 0.491 mmol) and Intermediate 9 (238 mg, 0.589 mmol) in dioxane (6 mL) were added K₂CO₃ (135 mg, 0.982 mmol) and Pd(dtbpf)Cl₂ (64.0 mg, 0.098 mmol). The mixture was stirred for 2 h at 100° C. under N₂. Water (15 mL) was added, and the mixture was extracted with DCM (3×15 mL). The combined organic layers were dried over Na₂SO₄ and concentrated. The crude product was purified by Prep-TLC with DCM:MeOH=12:1 to afford the title compound (220 mg, yield: 52%) as a yellow oil. LCMS: m/z=657 [M−H]

Step 4: Synthesis of tert-butyl (4-(5-chloro-3-fluoro-6-(trifluoromethyl)-1H-indazol-4-yl)-3-cyano-7-fluorobenzo[b]thiophen-2-yl)carbamate

To a stirred solution of the title compound from step 3 (200 mg, 0.303 mmol) in THF (5 mL) was added TBAF (1 mL, 1 M in THF). The mixture was stirred for 1 h at rt. The mixture was concentrated. Aqueous NaHCO₃ solution was added, and the mixture was extracted with EtOAc (3×10 mL). The combined organic layers were washed with water (3×10 mL) and dried over Na₂SO₄ and concentrated. The crude product was purified by Prep-TLC with DCM: MeOH=10:1 to afford the title compound (100 mg, yield: 62%) as a yellow oil. LCMS: m/z=527 [M−H]

Step 5: Synthesis of tert-butyl (4-(5-chloro-3-fluoro-1-(4-((S)-1-((S)-1-methylpyrrolidin-2-yl)ethoxy)-7,8-dihydro-5H-pyrano[4,3-d]pyrimidin-2-yl)-6-(trifluoromethyl)-1H-indazol-4-yl)-3-cyano-7-fluorobenzo[b]thiophen-2-yl)carbamate

To a stirred solution of the title compound from step 4 (90 mg, 0.170 mmol) and Intermediate 1 (50.6 mg, 0.170 mmol) in dioxane (2 mL) were added Cs₂CO₃ (110 mg, 0.340 mmol), Ephos (18.1 mg, 0.034 mmol) and Ephos Pd G4 (31.2 mg, 0.034 mmol). The mixture was stirred for 2 h at 80° C. under N₂. Water (10 mL) was added, and the mixture was extracted with DCM (3×10 mL). The combined organic layers were dried over Na₂SO₄ and concentrated. The crude product was purified by Prep-TLC with DCM:MeOH=12:1 to afford the title compound (120 mg, yield: 89%) as a yellow oil. LCMS: m/z=790 [M+H]

Step 6: Synthesis of 2-amino-4-(5-chloro-3-fluoro-1-(4-((S)-1-((S)-1-methylpyrrolidin-2-yl)ethoxy)-7,8-dihydro-5H-pyrano[4,3-d]pyrimidin-2-yl)-6-(trifluoromethyl)-1H-indazol-4-yl)-7-fluorobenzo[b]thiophene-3-carbonitrile

To a stirred solution of the title compound from step 5 (50 mg, 0.063 mmol) in DCM (1 mL) was added TFA (0.2 mL). The mixture was stirred for 1 h at rt and concentrated. Aqueous NaHCO₃ solution was added to adjust the pH to 7. The mixture was concentrated and purified by Prep-HPLC with following condition: Column: XBridge Prep OBD C18 Column, 30*150 mm, 5 μm; Mobile Phase A: Water (10 mmol/L NH₄HCO₃+0.05% NH₃·H₂O), Mobile Phase B: ACN; Flow rate: 60 mL/min; Gradient (B %): 61% B to 91% B in 8 min; Wave Length: 220 nm to afford the title compound (80 mg, yield: 83%) as a yellow solid. LCMS: m/z=690 [M+H]

Step 7: Synthesis of (S)-2-amino-4-(5-chloro-3-fluoro-1-(4-((S)-1-((S)-1-methylpyrrolidin-2-yl)ethoxy)-7,8-dihydro-5H-pyrano[4,3-d]pyrimidin-2-yl)-6-(trifluoromethyl)-1H-indazol-4-yl)-7-fluorobenzo[b]thiophene-3-carbonitrile and (R)-2-amino-4-(5-chloro-3-fluoro-1-(4-((S)-1-((S)-1-methylpyrrolidin-2-yl)ethoxy)-7,8-dihydro-5H-pyrano[4,3-d]pyrimidin-2-yl)-6-(trifluoromethyl)-1H-indazol-4-yl)-7-fluorobenzo[b]thiophene-3-carbonitrile

The title compound from Step 6 (80 mg, 0.115 mmol) was separated by Prep-Chiral-HPLC, Column: CHIRALPAK IG, 2*25 cm, 5 μm; Mobile Phase A: Hex (0.5% 2M NH₃-MeOH), Mobile Phase B: EtOH: DCM=1:1-HPLC; Flow rate: 20 mL/min; Gradient (B %): 20% B to 20% B in 16 min. This resulted in Peak 1, Example 160, (S)-2-amino-4-(5-chloro-3-fluoro-1-(4-((S)-1-((S)-1-methylpyrrolidin-2-yl)ethoxy)-7,8-dihydro-5H-pyrano[4,3-d]pyrimidin-2-yl)-6-(trifluoromethyl)-1H-indazol-4-yl)-7-fluorobenzo[b]thiophene-3-carbonitrile or (R)-2-amino-4-(5-chloro-3-fluoro-1-(4-((S)-1-((S)-1-methylpyrrolidin-2-yl)ethoxy)-7,8-dihydro-5H-pyrano[4,3-d]pyrimidin-2-yl)-6-(trifluoromethyl)-1H-indazol-4-yl)-7-fluorobenzo[b]thiophene-3-carbonitrile (26.4 mg; yield: 27%), as an off-white solid and Peak 2, Example 161 (R)-2-amino-4-(5-chloro-3-fluoro-1-(4-((S)-1-((S)-1-methylpyrrolidin-2-yl)ethoxy)-7,8-dihydro-5H-pyrano[4,3-d]pyrimidin-2-yl)-6-(trifluoromethyl)-1H-indazol-4-yl)-7-fluorobenzo[b]thiophene-3-carbonitrile or (S)-2-amino-4-(5-chloro-3-fluoro-1-(4-((S)-1-((S)-1-methylpyrrolidin-2-yl)ethoxy)-7,8-dihydro-5H-pyrano[4,3-d]pyrimidin-2-yl)-6-(trifluoromethyl)-1H-indazol-4-yl)-7-fluorobenzo[b]thiophene-3-carbonitrile (35.3 mg; yield: 36%) as an off-white solid.

Peak 1, Example 160, LCMS: m/z=690 [M+H]; ¹H NMR (400 MHz, DMSO-d₆) δ 9.26 (s, 1H), 8.17 (s, 2H), 7.47-7.44 (m, 1H), 7.24-7.16 (m, 1H), 5.37-5.31 (m, 1H), 4.65-4.55 (m, 2H), 4.00 (t, 2H), 3.03-2.89 (m, 4H), 2.51-2.33 (m, 4H), 1.94-1.69 (m, 4H), 1.37 (d, 3H).

Peak 2, Example 161, LCMS: m/z=690 [M+H]; ¹H NMR (400 MHz, DMSO-d₆) δ 9.25 (s, 1H), 8.17 (s, 2H), 7.45-7.40 (m, 1H), 7.24-7.13 (m, 1H), 5.39-5.33 (m, 1H), 4.67-4.52 (m, 2H), 4.00 (t, 2H), 3.00-2.67 (m, 4H), 2.39 (s, 3H), 2.26-2.19 (m, 1H), 1.91-1.66 (m, 4H), 1.36 (d, 3H).

### Example 162

### (S)-2-amino-4-(5-chloro-3-fluoro-6-methyl-1-(4-((S)-1-((S)-1-methylpyrrolidin-2-yl)ethoxy)-5,7-dihydrofuro[3,4-b]pyridin-2-yl)-1H-indazol-4-yl)-7-fluorobenzo[b]thiophene-3-carbonitrile or (R)-2-amino-4-(5-chloro-3-fluoro-6-methyl-1-(4-((S)-1-((S)-1-methylpyrrolidin-2-yl)ethoxy)-5,7-dihydrofuro[3,4-b]pyridin-2-yl)-1H-indazol-4-yl)-7-fluorobenzo[b]thiophene-3-carbonitrile

### And Example 163

### (R)-2-amino-4-(5-chloro-3-fluoro-6-methyl-1-(4-((S)-1-((S)-1-methylpyrrolidin-2-yl)ethoxy)-5,7-dihydrofuro[3,4-b]pyridin-2-yl)-1H-indazol-4-yl)-7-fluorobenzo[b]thiophene-3-carbonitrile or ( )₂-amino-4-(5-chloro-3-fluoro-6-methyl-1-(4-((S)-1-((S)-1-methylpyrrolidin-2-yl)ethoxy)-5,7-dihydrofuro[3,4-b]pyridin-2-yl)-1H-indazol-4-yl)-7-fluorobenzo[b]thiophene-3-carbonitrile

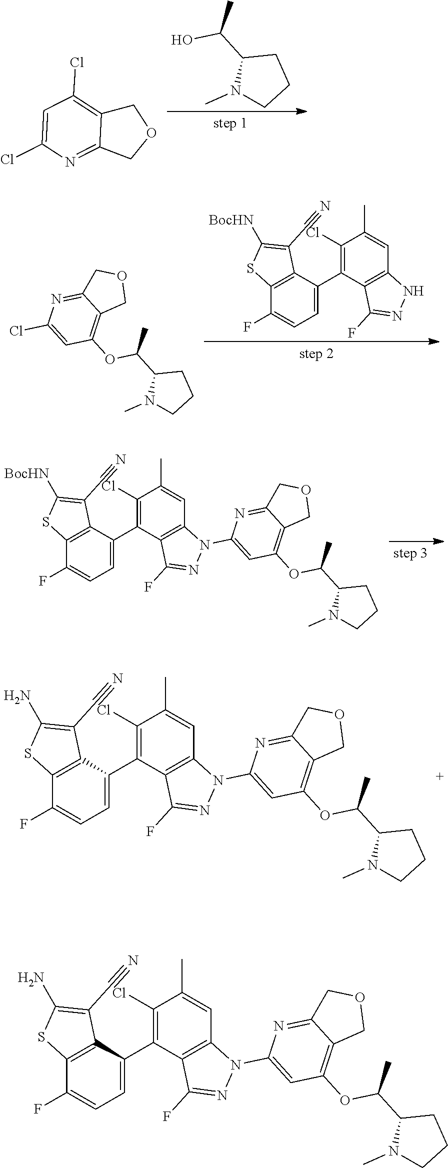

Step 1: Synthesis of 2-chloro-4-((S)-1-((S)-1-methylpyrrolidin-2-yl)ethoxy)-5,7-dihydrofuro[3,4-b]pyridine

To a stirred solution of 2,4-dichloro-5,7-dihydrofuro[3,4-b]pyridine (200 mg, 1.05 mmol) in THF (4 mL) was added(S)-1-((S)-1-methylpyrrolidin-2-yl)ethan-1-ol (202 mg, 1.57 mmol) and t-BuOK (235 mg, 2.10 mmol) at 0° C. The mixture was stirred overnight at 60° C. and then cooled to rt. Water (20 mL) was added and the resulting solution was extracted with DCM (3×20 mL). The combined organic layers were dried over Na₂SO₄ and concentrated. The crude product was purified by Prep-TLC with PE:EA=1:1 to give the title compound (150 mg, yield: 51%) as a yellow solid. LCMS: m/z=283 [M+H]

Step 2: Synthesis of tert-butyl (4-(5-chloro-3-fluoro-6-methyl-1-(4-((S)-1-((S)-1-methylpyrrolidin-2-yl)ethoxy)-5,7-dihydrofuro[3,4-b]pyridin-2-yl)-1H-indazol-4-yl)-3-cyano-7-fluorobenzo[b]thiophen-2-yl)carbamate

To a stirred solution of the title compound from step 1 (150 mg, 530 μmol) in dioxane (6 mL) were added the title compound from Examples 27 and 28, step 6 (251 mg, 530 μmol), Cs₂CO₃ (342 mg, 1.05 mmol) and GPhos Pd G6 (50.0 mg, 53.0 μmol) under N₂. The mixture was stirred for 2 hours at 100° C. and then cooled to rt. Water (10 mL) was added and the resulting solution was extracted with DCM (3×10 mL). The combined organic layers were dried over Na₂SO₄ and concentrated. The crude product was purified by silica gel column with DCM: MeOH=20:1 to afford the title compound (300 mg, yield: 79%) as a yellow solid. LCMS: m/z=721 [M+H]

Step 3: Synthesis of (S)-2-amino-4-(5-chloro-3-fluoro-6-methyl-1-(4-((S)-1-((S)-1-methylpyrrolidin-2-yl)ethoxy)-5,7-dihydrofuro[3,4-b]pyridin-2-yl)-1H-indazol-4-yl)-7-fluorobenzo[b]thiophene-3-carbonitrile and (R)-2-amino-4-(5-chloro-3-fluoro-6-methyl-1-(4-((S)-1-((S)-1-methylpyrrolidin-2-yl)ethoxy)-5,7-dihydrofuro[3,4-b]pyridin-2-yl)-1H-indazol-4-yl)-7-fluorobenzo[b]thiophene-3-carbonitrile

To a stirred solution of the title compound from step 2 (250 mg, 346 μmol) in DCM (4 mL) was added TFA (1 mL). The mixture was stirred overnight at rt. Saturated sodium bicarbonate solution was added to adjust the pH to 7. The mixture was extracted with DCM (3×10 mL). The combined organic layers were dried over Na₂SO₄ and concentrated. The crude product was purified by Prep-TLC with DCM:MeOH=10:1, followed by Prep-HPLC as following condition: Column: X Bridge Prep OBD C18 Column, 30*150 mm, 5 μm; Mobile Phase A: Water (10 mmol/L NH₄HCO₃+0.05% NH₃·H₂O), Mobile Phase B: ACN; Flow rate: 60 mL/min; Gradient (B %): 65% B to 85% B in 10 min to afford Peak 1, Example 162, (S)-2-amino-4-(5-chloro-3-fluoro-6-methyl-1-(4-((S)-1-((S)-1-methylpyrrolidin-2-yl)ethoxy)-5,7-dihydrofuro[3,4-b]pyridin-2-yl)-1H-indazol-4-yl)-7-fluorobenzo[b]thiophene-3-carbonitrile or (R)-2-amino-4-(5-chloro-3-fluoro-6-methyl-1-(4-((S)-1-((S)-1-methylpyrrolidin-2-yl)ethoxy)-5,7-dihydrofuro[3,4-b]pyridin-2-yl)-1H-indazol-4-yl)-7-fluorobenzo[b]thiophene-3-carbonitrile (83.3 mg, yield: 39%, 100% ee) as an off-white solid and Peak 2, Example 163, (R)-2-amino-4-(5-chloro-3-fluoro-6-methyl-1-(4-((S)-1-((S)-1-methylpyrrolidin-2-yl)ethoxy)-5,7-dihydrofuro[3,4-b]pyridin-2-yl)-1H-indazol-4-yl)-7-fluorobenzo[b]thiophene-3-carbonitrile or (S)-2-amino-4-(5-chloro-3-fluoro-6-methyl-1-(4-((S)-1-((S)-1-methylpyrrolidin-2-yl)ethoxy)-5,7-dihydrofuro[3,4-b]pyridin-2-yl)-1H-indazol-4-yl)-7-fluorobenzo[b]thiophene-3-carbonitrile (93.2 mg, yield: 44%, 98.1% ee) as an off-white solid.

Peak 1, Example 162, LCMS: m/z=621 [M+H], ¹H NMR (400 MHz, DMSO-d₆) δ 8.75 (s, 1H), 8.04 (s, 2H), 7.32 (dd, 1H), 7.27 (s, 1H), 7.15 (dd, 1H), 5.14-5.01 (m, 4H), 4.71 (p, 1H), 2.99-2.89 (m, 1H), 2.57-2.54 (m, 4H), 2.35 (s, 3H), 2.22 (t, 1H), 1.84 (dt, 1H), 1.73-1.60 (m, 3H), 1.26 (d, 3H).

Peak 2, Example 163, LCMS: m/z=621 [M+H], ¹H NMR (400 MHz, DMSO-d₆) δ 8.75 (s, 1H), 8.04 (s, 2H), 7.32 (dd, 1H), 7.28 (s, 1H), 7.15 (dd, 1H), 5.14-5.00 (m, 4H), 4.72 (p, 1H), 2.94-2.90 (m, 1H), 2.60-2.53 (m, 4H), 2.35 (s, 3H), 2.20 (d, 1H), 1.88-1.63 (m, 4H), 1.26 (d, 3H).

### Example 164

### (R)-2-amino-4-(5-chloro-3-fluoro-6-methyl-1-(4-((S)-1-((S)-1-methylpyrrolidin-2-yl)ethoxy)-1,3-dihydrofuro[3,4-c]pyridin-6-yl)-1H-indazol-4-yl)-7-fluorobenzo[b]thiophene-3-carbonitrile or (S)-2-amino-4-(5-chloro-3-fluoro-6-methyl-1-(4-((S)-1-((S)-1-methylpyrrolidin-2-yl)ethoxy)-1,3-dihydrofuro[3,4-c]pyridin-6-yl)-1H-indazol-4-yl)-7-fluorobenzo[b]thiophene-3-carbonitrile

### And Example 165

### (S)-2-amino-4-(5-chloro-3-fluoro-6-methyl-1-(4-((S)-1-((S)-1-methylpyrrolidin-2-yl)ethoxy)-1,3-dihydrofuro[3,4-c]pyridin-6-yl)-1H-indazol-4-yl)-7-fluorobenzo[b]thiophene-3-carbonitrile or (R)-2-amino-4-(5-chloro-3-fluoro-6-methyl-1-(4-((S)-1-((S)-1-methylpyrrolidin-2-yl)ethoxy)-1,3-dihydrofuro[3,4-c]pyridin-6-yl)-1H-indazol-4-yl)-7-fluorobenzo[b]thiophene-3-carbonitrile

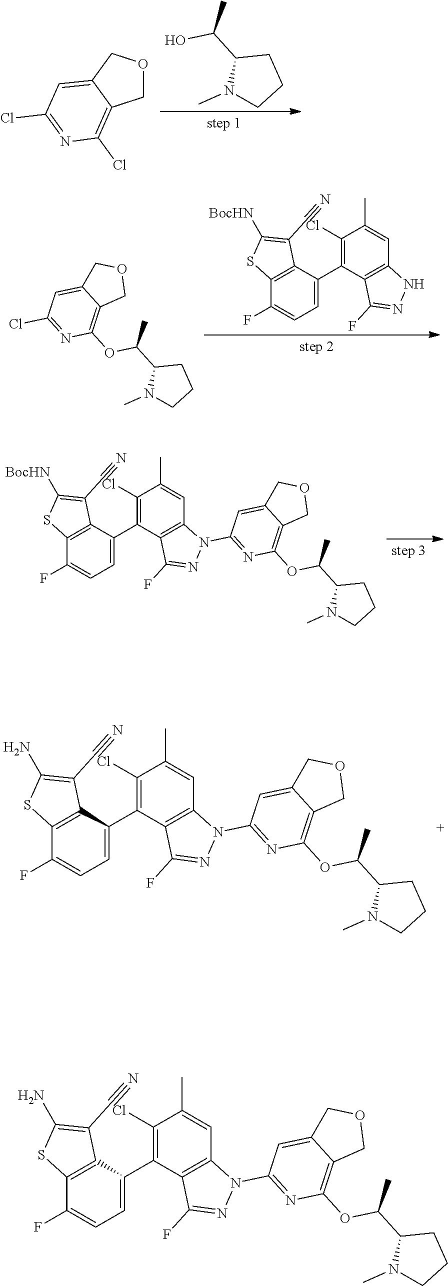

Step 1: Synthesis of 6-chloro-4-((S)-1-((S)-1-methylpyrrolidin-2-yl)ethoxy)-1,3-dihydrofuro[3,4-c]pyridine

To a stirred solution of 4,6-dichloro-1,3-dihydrofuro[3,4-c]pyridine (300 mg, 1.57 mmol) and (S)-1-((S)-1-methylpyrrolidin-2-yl)ethan-1-ol (202 mg, 1.57 mmol) in THF (6 mL) was added NaH (125 mg, 3.14 mmol) at 0° C. The mixture was stirred for 1 hour at 50° C. and cooled to rt. Water (10 mL) was added and the resulting solution was extracted with DCM (3×10 mL). The combined organic layers were dried over Na₂SO₄ and concentrated. The crude product was purified by Prep-TLC with PE:EA=1:1 to give the title compound (150 mg; yield: 34%) as a brown oil. LCMS: m/z=283 [M+H].

Step 2: Synthesis of tert-butyl (4-(5-chloro-3-fluoro-6-methyl-1-(4-((S)-1-((S)-1-methylpyrrolidin-2-yl)ethoxy)-1,3-dihydrofuro[3,4-c]pyridin-6-yl)-1H-indazol-4-yl)-3-cyano-7-fluorobenzo[b]thiophen-2-yl)carbamate

A mixture of the title compound from step 1 (130 mg, 459 μmol), the title compound from Examples 27 and 28, step 6, Gphos Pd G6 (43.3 mg, 45.9 μmol) and Cs₂CO₃ (298 mg, 918 μmol) in dioxane (3 mL) under N₂ was stirred for 2 hours at 80° C. and then cooled to rt. The mixture was filtered, and the filter cake was washed with DCM. The filtrate was concentrated, and the crude product was purified by Prep-TLC with DCM:MeOH=15:1 to give the title compound (90 mg; yield: 40%) as a brown solid. LCMS: m/z=721 [M+H].

Step 3: Synthesis of (R)-2-amino-4-(5-chloro-3-fluoro-6-methyl-1-(4-((S)-1-((S)-1-methylpyrrolidin-2-yl)ethoxy)-1,3-dihydrofuro[3,4-c]pyridin-6-yl)-1H-indazol-4-yl)-7-fluorobenzo[b]thiophene-3-carbonitrile and (S)-2-amino-4-(5-chloro-3-fluoro-6-methyl-1-(4-((S)-1-((S)-1-methylpyrrolidin-2-yl)ethoxy)-1,3-dihydrofuro[3,4-c]pyridin-6-yl)-1H-indazol-4-yl)-7-fluorobenzo[b]thiophene-3-carbonitrile

To a stirred solution of the title compound from step 2 (120 mg, 166 μmol) in DCM (1 mL) was added TFA (0.3 mL) and stirred for 2 hours at rt. The mixture was adjusted to pH=7 with aqueous NaHCO₃ solution and concentrated. The crude product was purified by Prep-HPLC: Column: Xselect CSH C18 OBD Column 30*150 mm 5 μm; Mobile Phase A: Water (0.1% FA), Mobile Phase B: ACN; Flow rate: 60 mL/min; Gradient (B %): 23% B to 53% B in 10 min; followed by Prep-CHIRAL-HPLC, Column: CHIRALPAK IK 3*25 cm, 5 μm; Mobile Phase A: Hex (0.5% 2 M NH₃-MeOH), Mobile Phase B: EtOH: DCM=1:1-HPLC; Flow rate: 40 mL/min; Gradient (B %): 30% B to 30% B in 13 min to give Peak 1, Example 164, (R)-2-amino-4-(5-chloro-3-fluoro-6-methyl-1-(4-((S)-1-((S)-1-methylpyrrolidin-2-yl)ethoxy)-1,3-dihydrofuro[3,4-c]pyridin-6-yl)-1H-indazol-4-yl)-7-fluorobenzo[b]thiophene-3-carbonitrile or (S)-2-amino-4-(5-chloro-3-fluoro-6-methyl-1-(4-((S)-1-((S)-1-methylpyrrolidin-2-yl)ethoxy)-1,3-dihydrofuro[3,4-c]pyridin-6-yl)-1H-indazol-4-yl)-7-fluorobenzo[b]thiophene-3-carbonitrile (29.2 mg, yield: 28%) as a white solid and Peak 2, Example 165, (S)-2-amino-4-(5-chloro-3-fluoro-6-methyl-1-(4-((S)-1-((S)-1-methylpyrrolidin-2-yl)ethoxy)-1,3-dihydrofuro[3,4-c]pyridin-6-yl)-1H-indazol-4-yl)-7-fluorobenzo[b]thiophene-3-carbonitrile or (R)-2-amino-4-(5-chloro-3-fluoro-6-methyl-1-(4-((S)-1-((S)-1-methylpyrrolidin-2-yl)ethoxy)-1,3-dihydrofuro[3,4-c]pyridin-6-yl)-1H-indazol-4-yl)-7-fluorobenzo[b]thiophene-3-carbonitrile (27.7 mg, yield: 27%) as a white solid.

Peak 1, Example 164, LCMS: m/z=621 [M+H], ¹H NMR (400 MHz, DMSO-d₆) δ 8.69 (s, 1H), 8.07 (s, 2H), 7.37-7.26 (m, 2H), 7.15 (dd, 1H), 5.39 (p, 1H), 5.07 (d, 2H), 4.97 (qt, 2H), 3.03-2.94 (m, 1H), 2.62 (dt, 1H), 2.57 (s, 3H), 2.39 (s, 3H), 2.22 (q, 1H), 1.90 (dq, 1H), 1.71 (dtt, 3H), 1.42 (d, 3H).

Peak 2, Example 165, LCMS: m/z=621 [M+H], ¹H NMR (400 MHz, DMSO-d6) δ 8.70 (s, 1H), 8.06 (s, 2H), 7.38-7.27 (m, 2H), 7.16 (dd, 1H), 5.36 (p, 1H), 5.14-4.87 (m, 4H), 3.01 (dd, 1H), 2.65 (dd, 1H), 2.57 (s, 3H), 2.40 (s, 3H), 2.24 (q, 1H), 1.92 (tt, 1H), 1.71 (td, 3H), 1.41 (d, 3H).

### Example 166

### (S)-2-amino-4-(5-chloro-3-fluoro-6-methyl-1-((S)-5-methyl-4-((S)-1-((S)-1-methylpyrrolidin-2-yl)ethoxy)-5,7-dihydrofuro[3,4-d]pyrimidin-2-yl)-1H-indazol-4-yl)-7-fluorobenzo[b]thiophene-3-carbonitrile or (R)-2-amino-4-(5-chloro-3-fluoro-6-methyl-1-((S)-5-methyl-4-((S)-1-((S)-1-methylpyrrolidin-2-yl)ethoxy)-5,7-dihydrofuro[3,4-d]pyrimidin-2-yl)-1H-indazol-4-yl)-7-fluorobenzo[b]thiophene-3-carbonitrile or (S)-2-amino-4-(5-chloro-3-fluoro-6-methyl-1-((R)-5-methyl-4-((S)-1-((S)-1-methylpyrrolidin-2-yl)ethoxy)-5,7-dihydrofuro[3,4-d]pyrimidin-2-yl)-1H-indazol-4-yl)-7-fluorobenzo[b]thiophene-3-carbonitrile or (R)-2-amino-4-(5-chloro-3-fluoro-6-methyl-1-((R)-5-methyl-4-((S)-1-((S)-1-methylpyrrolidin-2-yl)ethoxy)-5,7-dihydrofuro[3,4-d]pyrimidin-2-yl)-1H-indazol-4-yl)-7-fluorobenzo[b]thiophene-3-carbonitrile

### And Example 167

### (R)-2-amino-4-(5-chloro-3-fluoro-6-methyl-1-((S)-5-methyl-4-((S)-1-((S)-1-methylpyrrolidin-2-yl)ethoxy)-5,7-dihydrofuro[3,4-d]pyrimidin-2-yl)-1H-indazol-4-yl)-7-fluorobenzo[b]thiophene-3-carbonitrile or (S)-2-amino-4-(5-chloro-3-fluoro-6-methyl-1-((S)-5-methyl-4-((S)-1-((S)-1-methylpyrrolidin-2-yl)ethoxy)-5,7-dihydrofuro[3,4-d]pyrimidin-2-yl)-1H-indazol-4-yl)-7-fluorobenzo[b]thiophene-3-carbonitrile or (S)-2-amino-4-(5-chloro-3-fluoro-6-methyl-1-((R)-5-methyl-4-((S)-1-((S)-1-methylpyrrolidin-2-yl)ethoxy)-5,7-dihydrofuro[3,4-d]pyrimidin-2-yl)-1H-indazol-4-yl)-7-fluorobenzo[b]thiophene-3-carbonitrile or (R)-2-amino-4-(5-chloro-3-fluoro-6-methyl-1-((R)-5-methyl-4-((S)-1-((S)-1-methylpyrrolidin-2-yl)ethoxy)-5,7-dihydrofuro[3,4-d]pyrimidin-2-yl)-1H-indazol-4-yl)-7-fluorobenzo[b]thiophene-3-carbonitrile

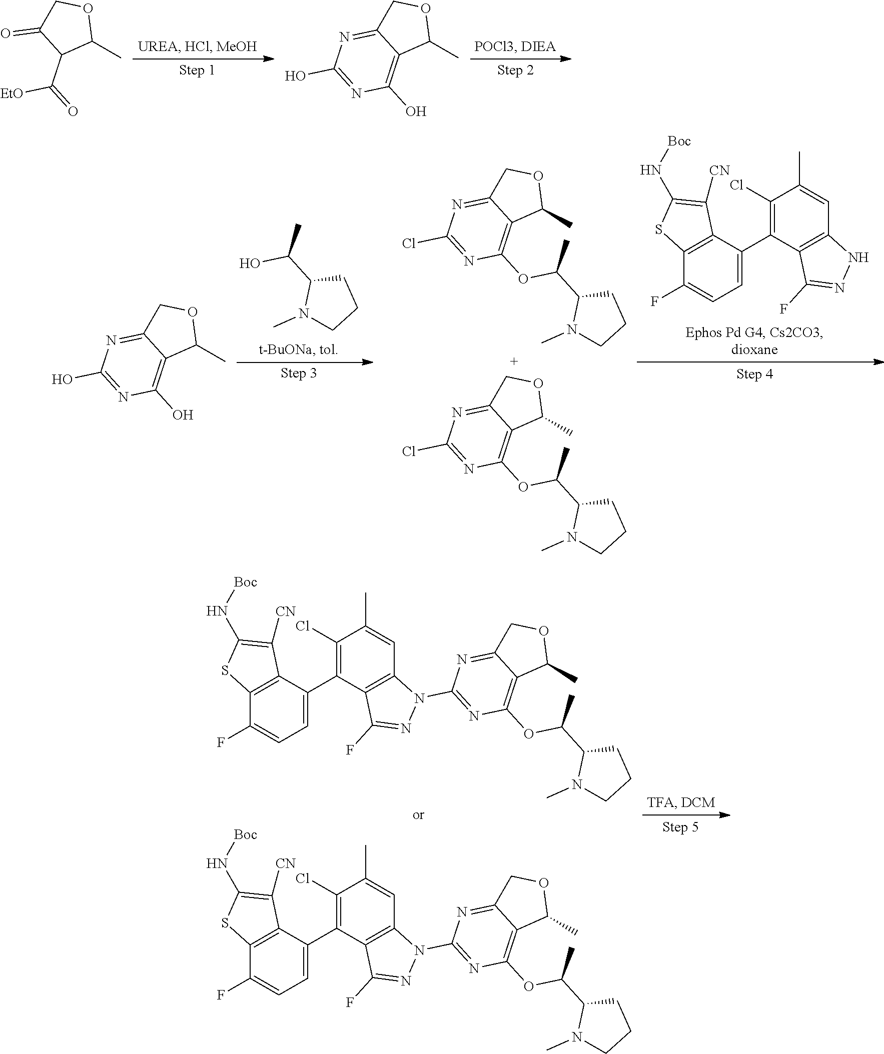

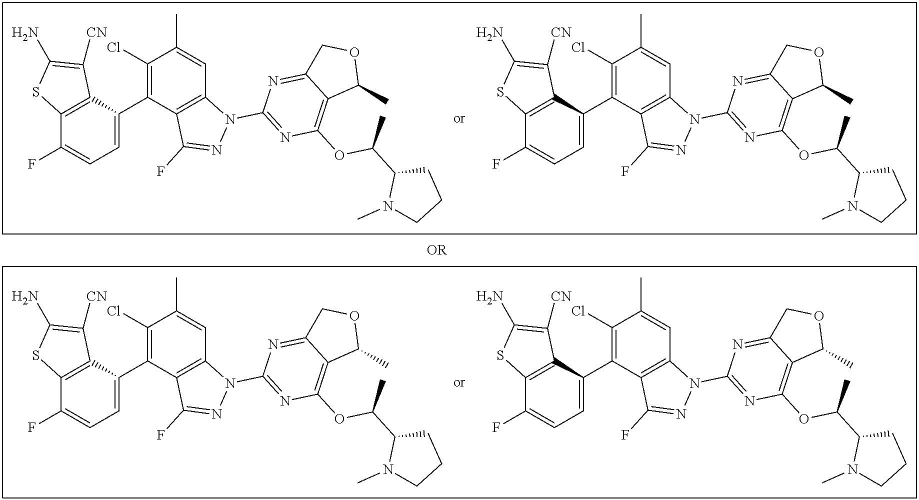

Step 1: Synthesis of 5-methyl-5,7-dihydrofuro[3,4-d]pyrimidine-2,4-diol

To a solution of ethyl 2-methyl-4-oxotetrahydrofuran-3-carboxylate (1.2 g, 6.97 mmol) in MeOH (6 mL) was added urea (628 mg, 10.5 mmol) and hydrochloric acid (12 M, 300 μL). The mixture was stirred at 70° C. for 12 h. The reaction mixture was cooled to 0° C. for 15 minutes, then mixture was filtered and the solid was quenched by addition sodium hydroxide solution (2 M, 1.2 mL), the resulting mixture was refluxed for 1 h. The solution was then cooled to rt and hydrochloric acid was slowly added which resulted in a precipitate. The mixture was filtered, the solid was concentrated under reduced pressure to give the title compound (420 mg, 36% yield) as a white solid. ¹H NMR (400 MHz, DMSO-d6) δ 11.26 (s, 1H), 10.99 (s, 1H), 5.09-4.95 (m, 1H), 4.79-4.69 (m, 1H), 4.69-4.58 (m, 1H), 1.32 (d, 3H)

Step 2: Synthesis of 2,4-dichloro-5-methyl-5,7-dihydrofuro[3,4-d]pyrimidine

A mixture of the title compound from Step 1 (420 mg, 2.50 mmol), POCl₃ (4.2 mL) and DIPEA (1.61 g, 12.5 mmol) was degassed and purged with N₂, and stirred at 110° C. for 12 h under N₂. The mixture was poured into ice water (20 mL) then extracted with extracted with EtOAc (20 mL×3). The combined organic layers were washed with brine (20 mL×3), dried over anhydrous Na₂SO₄, filtered and concentrated under reduced pressure. The residue was purified by column chromatography (SiO₂, hexanes/EtOAc=100/1 to 4/1) to give the title compound (240 mg, 42% yield) as a yellow oil. ¹1H NMR (400 MHz, DMSO-d6) δ 5.48-5.40 (m, 1H), 5.13-5.04 (m, 1H), 5.02-4.94 (m, 1H), 1.51 (d, 3H)

Step 3: Synthesis of (S)-2-chloro-5-methyl-4-((S)-1-((S)-1-methylpyrrolidin-2-yl)ethoxy)-5,7-dihydrofuro[3,4-d]pyrimidine and (R)-2-chloro-5-methyl-4-((S)-1-((S)-1-methylpyrrolidin-2-yl)ethoxy)-5,7-dihydrofuro[3,4-d]pyrimidine

To a mixture of the title compound from Step 2 (290 mg, 1.41 mmol) and (1S)-1-[(2S)-1-methylpyrrolidin-2-yl]ethanol (201 mg, 1.56 mmol) in toluene (5 mL) was added sodium tert-butoxide (204 mg, 2.12 mmol). The mixture was stirred at rt for 0.5 h then diluted with water (20 mL) and extracted with EtOAc (20 mL×3). The combined organic layers were washed with brine (20 mL×3), dried over anhydrous Na₂SO₄, filtered and concentrated under reduced pressure. The residue was purified by prep-HPLC (column: Phenomenex Luna C18 150*25 mm*10 um; mobile phase: [H2O(0.1% TFA)-ACN]; gradient:0%-25% B over 15.0 min) to give peak 1, (S)-2-chloro-5-methyl-4-((S)-1-((S)-1-methylpyrrolidin-2-yl)ethoxy)-5,7-dihydrofuro[3,4-d]pyrimidine or (R)-2-chloro-5-methyl-4-((S)-1-((S)-1-methylpyrrolidin-2-yl)ethoxy)-5,7-dihydrofuro[3,4-d]pyrimidine (150 mg, 84% yield) as a white solid, LCMS: m/z=297.1 [M+H] and peak 2, (R)-2-chloro-5-methyl-4-((S)-1-((S)-1-methylpyrrolidin-2-yl)ethoxy)-5,7-dihydrofuro[3,4-d]pyrimidine or (S)-2-chloro-5-methyl-4-((S)-1-((S)-1-methylpyrrolidin-2-yl)ethoxy)-5,7-dihydrofuro[3,4-d]pyrimidine, which was further purified by SFC (column: DAICEL CHIRALPAK IG 250 mm*30 mm*5 μm; mobile phase: [CO2-MeOH (0.2% NH3H2O)]; B %: 15%, isocratic elution mode) to afford a white solid (40 mg, 23% yield), LCMS: m/z=297.1 [M+H].

Step 4: Synthesis of tert-butyl (4-(5-chloro-3-fluoro-6-methyl-1-((S)-5-methyl-4-((S)-1-((S)-1-methylpyrrolidin-2-yl)ethoxy)-5,7-dihydrofuro[3,4-d]pyrimidin-2-yl)-1H-indazol-4-yl)-3-cyano-7-fluorobenzo[b]thiophen-2-yl)carbamate or tert-butyl (4-(5-chloro-3-fluoro-6-methyl-1-((R)-5-methyl-4-((S)-1-((S)-1-methylpyrrolidin-2-yl)ethoxy)-5,7-dihydrofuro[3,4-d]pyrimidin-2-yl)-1H-indazol-4-yl)-3-cyano-7-fluorobenzo[b]thiophen-2-yl)carbamate

To a stirred solution of Peak 1 from Step 3 (130 mg, 437 μmol), the title compound from Examples 27 and 28, Step 6 (207 mg, 437 μmol) and Cs₂CO₃ (427 mg, 1.31 mmol) in dioxane (3 mL) were added Ephos Pd G4 (40.1 mg,43.7 μmol) under N₂. The reaction mixture was stirred for 0.5 h at 80° C., cooled to rt, diluted with water (10 mL) and extracted with EtOAc (15 mL×3). The combined organic layers were washed with brine (15 mL), dried over anhydrous Na₂SO₄, filtered and concentrated under reduced pressure. The residue was purified by prep-HPLC (column: Phenomenex Luna C18 150*25 mm*10 um; mobile phase: [H2O(0.1% TFA)-ACN]; gradient:35%-65% B over 15.0 min) to give the title compound (190 mg, 56% yield) as a white solid. LCMS: m/z=736.4 [M+H].

Step 5: Synthesis of (S)-2-amino-4-(5-chloro-3-fluoro-6-methyl-1-((S)-5-methyl-4-((S)-1-((S)-1-methylpyrrolidin-2-yl)ethoxy)-5,7-dihydrofuro[3,4-d]pyrimidin-2-yl)-1H-indazol-4-yl)-7-fluorobenzo[b]thiophene-3-carbonitrile and (R)-2-amino-4-(5-chloro-3-fluoro-6-methyl-1-((S)-5-methyl-4-((S)-1-((S)-1-methylpyrrolidin-2-yl)ethoxy)-5,7-dihydrofuro[3,4-d]pyrimidin-2-yl)-1H-indazol-4-yl)-7-fluorobenzo[b]thiophene-3-carbonitrile or (S)-2-amino-4-(5-chloro-3-fluoro-6-methyl-1-((R)-5-methyl-4-((S)-1-((S)-1-methylpyrrolidin-2-yl)ethoxy)-5,7-dihydrofuro[3,4-d]pyrimidin-2-yl)-1H-indazol-4-yl)-7-fluorobenzo[b]thiophene-3-carbonitrile and (R)-2-amino-4-(5-chloro-3-fluoro-6-methyl-1-((R)-5-methyl-4-((S)-1-((S)-1-methylpyrrolidin-2-yl)ethoxy)-5,7-dihydrofuro[3,4-d]pyrimidin-2-yl)-1H-indazol-4-yl)-7-fluorobenzo[b]thiophene-3-carbonitrile

To a stirred solution of the title compound from step 4 (180 mg, 244 μmol) in DCM (6 mL) was added TFA (2 mL) and the reaction mixture was stirred for 0.5 h at rt and then concentrated under reduced pressure. The residue was purified by prep-HPLC (column: Phenomenex Luna C18 150*25 mm*10 um; mobile phase: [H2O(0.1% TFA)-ACN]; gradient:20%-50% B over 15.0 min) to give Peak 1, Example 166 (49 mg, 31% yield) as a pink solid and Peak 2, Example 167, (29.4 mg, 19% yield) as a pink solid.

Example 166, LCMS: m/z=635.2 [M+H]⁺, 1H NMR (400 MHz, DMSO-d6) δ 9.99 (s, 1H), 8.69 (s, 1H), 8.06 (s, 2H), 7.31 (dd, 1H), 7.16 (t, 1H), 5.67-5.56 (m, 1H), 5.46-5.34 (m, 1H), 5.18-5.06 (m, 1H), 5.04-4.93 (m, 1H), 3.86 (dd, 1H), 3.61 (dd, 1H), 3.24-3.14 (m, 1H), 2.96 (d, 3H), 2.61 (s, 3H), 2.31 (d, 1H), 2.16-2.05 (m, 1H), 2.02-1.84 (m, 2H), 1.49 (dd, 6H).

Example 167, LCMS: m/z=635.2 [M+H]⁺, 1H NMR (400 MHz, DMSO-d6) δ 10.03 (s, 1H), 8.68 (s, 1H), 8.06 (s, 2H), 7.32 (dd, 1H), 7.16 (dd, 1H), 5.63 (dd, 1H), 5.47-5.36 (m, 1H), 5.23-5.08 (m, 1H), 5.05-4.90 (m, 1H), 3.85 (dd, 1H), 3.58 (s, 1H), 3.19 (d, 1H), 2.95 (d, 3H), 2.61 (s, 3H), 2.35-2.24 (m, 1H), 2.16-2.04 (m, 1H), 2.02-1.85 (m, 2H), 1.49 (t, 6H).

### Example 168

### (S)-2-amino-4-(5-chloro-3-fluoro-6-methyl-1-((R)-5-methyl-4-((S)-1-((S)-1-methylpyrrolidin-2-yl)ethoxy)-5,7-dihydrofuro[3,4-d]pyrimidin-2-yl)-1H-indazol-4-yl)-7-fluorobenzo[b]thiophene-3-carbonitrile or (R)-2-amino-4-(5-chloro-3-fluoro-6-methyl-1-((R)-5-methyl-4-((S)-1-((S)-1-methylpyrrolidin-2-yl)ethoxy)-5,7-dihydrofuro[3,4-d]pyrimidin-2-yl)-1H-indazol-4-yl)-7-fluorobenzo[b]thiophene-3-carbonitrile or (S)-2-amino-4-(5-chloro-3-fluoro-6-methyl-1-((S)-5-methyl-4-((S)-1-((S)-1-methylpyrrolidin-2-yl)ethoxy)-5,7-dihydrofuro[3,4-d]pyrimidin-2-yl)-1H-indazol-4-yl)-7-fluorobenzo[b]thiophene-3-carbonitrile or (R)-2-amino-4-(5-chloro-3-fluoro-6-methyl-1-((S)-5-methyl-4-((S)-1-((S)-1-methylpyrrolidin-2-yl)ethoxy)-5,7-dihydrofuro[3,4-d]pyrimidin-2-yl)-1H-indazol-4-yl)-7-fluorobenzo[b]thiophene-3-carbonitrile

### And Example 169

### (R)-2-amino-4-(5-chloro-3-fluoro-6-methyl-1-((R)-5-methyl-4-((S)-1-((S)-1-methylpyrrolidin-2-yl)ethoxy)-5,7-dihydrofuro[3,4-d]pyrimidin-2-yl)-1H-indazol-4-yl)-7-fluorobenzo[b]thiophene-3-carbonitrile or (S)-2-amino-4-(5-chloro-3-fluoro-6-methyl-1-((R)-5-methyl-4-((S)-1-((S)-1-methylpyrrolidin-2-yl)ethoxy)-5,7-dihydrofuro[3,4-d]pyrimidin-2-yl)-1H-indazol-4-yl)-7-fluorobenzo[b]thiophene-3-carbonitrile or (S)-2-amino-4-(5-chloro-3-fluoro-6-methyl-1-((S)-5-methyl-4-((S)-1-((S)-1-methylpyrrolidin-2-yl)ethoxy)-5,7-dihydrofuro[3,4-d]pyrimidin-2-yl)-1H-indazol-4-yl)-7-fluorobenzo[b]thiophene-3-carbonitrile or (R)-2-amino-4-(5-chloro-3-fluoro-6-methyl-1-((S)-5-methyl-4-((S)-1-((S)-1-methylpyrrolidin-2-yl)ethoxy)-5,7-dihydrofuro[3,4-d]pyrimidin-2-yl)-1H-indazol-4-yl)-7-fluorobenzo[b]thiophene-3-carbonitrile

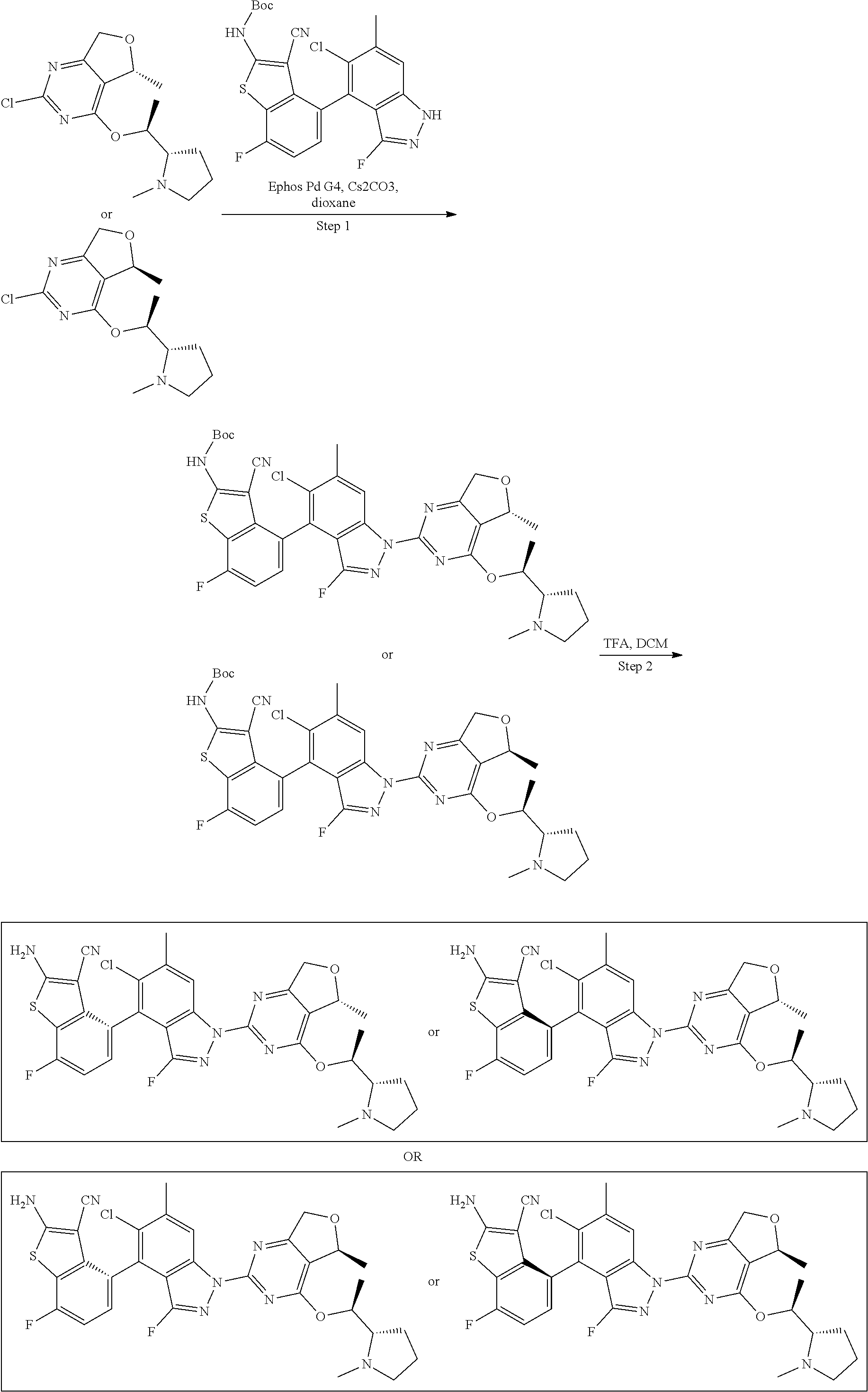

Step 1: Synthesis of tert-butyl (4-(5-chloro-3-fluoro-6-methyl-1-((R)-5-methyl-4-((S)-1-((S)-1-methylpyrrolidin-2-yl)ethoxy)-5,7-dihydrofuro[3,4-d]pyrimidin-2-yl)-1H-indazol-4-yl)-3-cyano-7-fluorobenzo[b]thiophen-2-yl)carbamate or tert-butyl (4-(5-chloro-3-fluoro-6-methyl-1-((S)-5-methyl-4-((S)-1-((S)-1-methylpyrrolidin-2-yl)ethoxy)-5,7-dihydrofuro[3,4-d]pyrimidin-2-yl)-1H-indazol-4-yl)-3-cyano-7-fluorobenzo[b]thiophen-2-yl)carbamate

To a stirred mixture of Peak 2 from Step 3 (40 mg, 134.33 μmol), the title compound from Examples 27 and 28, Step 6 (63.8 mg, 134 μmol) and Cs₂CO₃ (131 mg, 403 μmol) in dioxane (1 mL) was added Ephos Pd G4 (12.3 mg, 13.4 μmol) under N₂. The reaction mixture was stirred for 0.5 h at 80° C., cooled to rt, diluted with water (5 mL) and extracted with EtOAc (5 mL×3). The combined organic layers were washed with brine (5 mL), dried over anhydrous Na₂SO₄, filtered and concentrated under reduced pressure. The residue was purified by prep-HPLC (column: Phenomenex Luna C18 150*25 mm*10 um; mobile phase: [H2O(0.1% TFA)-ACN]; gradient:30%-60% B over 15.0 min) to give the title compound (75 mg, 68% yield) as a white solid. LCMS: m/z=735.2 [M+H].

Step 2: Synthesis of (S)-2-amino-4-(5-chloro-3-fluoro-6-methyl-1-((R)-5-methyl-4-((S)-1-((S)-1-methylpyrrolidin-2-yl)ethoxy)-5,7-dihydrofuro[3,4-d]pyrimidin-2-yl)-1H-indazol-4-yl)-7-fluorobenzo[b]thiophene-3-carbonitrile and (R)-2-amino-4-(5-chloro-3-fluoro-6-methyl-1-((R)-5-methyl-4-((S)-1-((S)-1-methylpyrrolidin-2-yl)ethoxy)-5,7-dihydrofuro[3,4-d]pyrimidin-2-yl)-1H-indazol-4-yl)-7-fluorobenzo[b]thiophene-3-carbonitrile or (S)-2-amino-4-(5-chloro-3-fluoro-6-methyl-1-((S)-5-methyl-4-((S)-1-((S)-1-methylpyrrolidin-2-yl)ethoxy)-5,7-dihydrofuro[3,4-d]pyrimidin-2-yl)-1H-indazol-4-yl)-7-fluorobenzo[b]thiophene-3-carbonitrile and (R)-2-amino-4-(5-chloro-3-fluoro-6-methyl-1-((S)-5-methyl-4-((S)-1-((S)-1-methylpyrrolidin-2-yl)ethoxy)-5,7-dihydrofuro[3,4-d]pyrimidin-2-yl)-1H-indazol-4-yl)-7-fluorobenzo[b]thiophene-3-carbonitrile

To a stirred solution of the title compound from step 1 (65.0 mg, 88.3 μmol) in DCM (3 mL) was added TFA (1 mL) and the reaction mixture was stirred for 0.5 h at rt and then concentrated under reduced pressure. The residue was purified by prep-HPLC (column: Phenomenex Luna C18 150*25 mm*10 um; mobile phase: [H2O(0.1% TFA)-ACN]; gradient:23%-53% B over 15.0 min) to give Peak 1, Example 168 (22.7 mg, 40% yield) as an off-white solid, and Peak 2, Example 169, (23.1 mg, 41% yield) as an off-white solid.

Example 168, LCMS: m/z=635.2 [M+H]⁺, 1H NMR (400 MHz, DMSO-d6) δ 10.05-9.86 (m, 1H), 8.70 (s, 1H), 8.06 (s, 2H), 7.31 (dd, 1H), 7.16 (t, 1H), 5.54-5.38 (m, 2H), 5.18-5.08 (m, 1H), 5.06-4.94 (m, 1H), 3.89-3.80 (m, 1H), 3.58 (d, 1H), 3.19 (dd, 1H), 2.96 (d, 3H), 2.61 (s, 3H), 2.31 (d, 1H), 2.10 (dd, 1H), 2.03-1.90 (m, 2H), 1.54 (d, 3H), 1.50 (d, 3H).

Example 169, LCMS: m/z=635.2 [M+H]⁺, 1H NMR (400 MHz, DMSO-d6) δ 10.12-9.96 (m, 1H), 8.68 (s, 1H), 8.06 (s, 2H), 7.32 (dd, 1H), 7.16 (t, 1H), 5.54-5.37 (m, 2H), 5.20-5.09 (m, 1H), 5.07-4.97 (m, 1H), 3.83 (d, 1H), 3.60-3.55 (m, 1H), 3.23-3.11 (m, 1H), 2.95 (d, 3H), 2.60 (s, 3H), 2.35-2.27 (m, 1H), 2.08 (d, 1H), 1.95 (dd, 2H), 1.54 (d, 3H), 1.50 (d, 3H).

### Example 170

### (S)-2-amino-4-(5-chloro-3-fluoro-6-methyl-1-(4-(1-methylpiperidin-4-yl)-7,8-dihydro-5H-pyrano[4,3-d]pyrimidin-2-yl)-1H-indazol-4-yl)-7-fluorobenzo[b]thiophene-3-carbonitrile or (R)-2-amino-4-(5-chloro-3-fluoro-6-methyl-1-(4-(1-methylpiperidin-4-yl)-7,8-dihydro-5H-pyrano[4,3-d]pyrimidin-2-yl)-1H-indazol-4-yl)-7-fluorobenzo[b]thiophene-3-carbonitrile

### And Example 171

### (R)-2-amino-4-(5-chloro-3-fluoro-6-methyl-1-(4-(1-methylpiperidin-4-yl)-7,8-dihydro-5H-pyrano[4,3-d]pyrimidin-2-yl)-1H-indazol-4-yl)-7-fluorobenzo[b]thiophene-3-carbonitrile or (S)-2-amino-4-(5-chloro-3-fluoro-6-methyl-1-(4-(1-methylpiperidin-4-yl)-7,8-dihydro-5H-pyrano[4,3-d]pyrimidin-2-yl)-1H-indazol-4-yl)-7-fluorobenzo[b]thiophene-3-carbonitrile

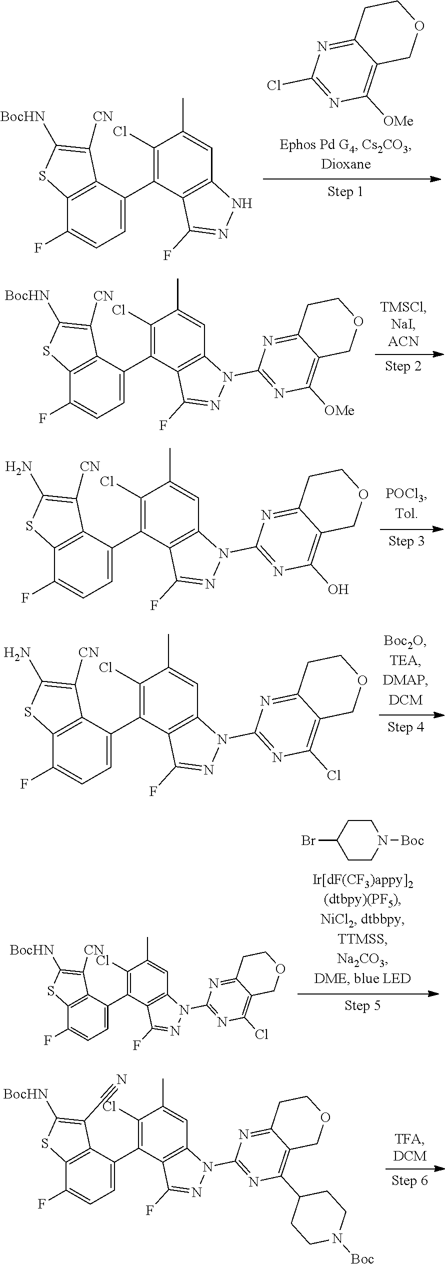

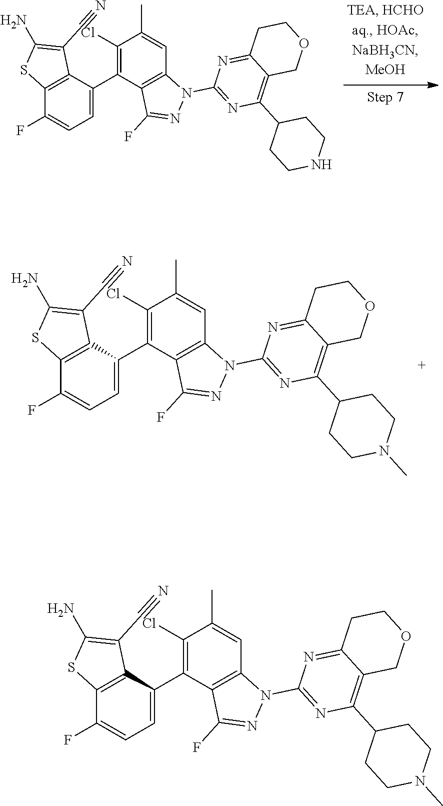

Step 1: Synthesis of tert-butyl (4-(5-chloro-3-fluoro-1-(4-methoxy-7,8-dihydro-5H-pyrano[4,3-d]pyrimidin-2-yl)-6-methyl-1H-indazol-4-yl)-3-cyano-7-fluorobenzo[b]thiophen-2-yl)carbamate

To a mixture of the title compound title compound from Example 27 and 28. Step 6 (78.5 g, 165 mmol), 2-chloro-7,8-dihydro-4-methoxy-5H-pyrano[4,3-d]pyrimidine (33.2 g, 165 mmol), Cs₂CO₃ (108 g, 331 mmol) in dioxane (785 mL) was added EPhos Pd G4 (15.2 g, 16.5 mmol) at rt and under N₂. The mixture was stirred at 85° C. for 2 h then poured into water (1.5 L) and 2-Me-THF (1.0 L) upon cooling to rt. The mixture was stirred for 0.5 h at rt then filtered. The filter cake was washed with water (2×300 mL) then azeotroped with toluene (4×500 mL) under reduced pressure to afford the title compound (64.0 g, 59.5% yield) as a brown solid. LCMS: m/z=639.2 [M+H]⁺.

Step 2: Synthesis of 2-amino-4-(5-chloro-3-fluoro-1-(4-hydroxy-7,8-dihydro-5H-pyrano[4,3-d]pyrimidin-2-yl)-6-methyl-1H-indazol-4-yl)-7-fluorobenzo[b]thiophene-3-carbonitrile

A mixture of the title compound from Step 1 (60.0 g, 93.9 mmol), NaI (21.1 g, 141 mmol), TMSCl (15.3 g, 17.9 mL, 141 mmol) in ACN (600 mL) was stirred at 75° C. for 3 h. The mixture was poured into water (1.5 L) upon cooling to rt and then filtered. The filter cake was washed with water (2×300 mL) then azeotroped with toluene (4×500 mL) under reduced pressure to afford the title compound (45.0 g, 69.8% yield) as a brown solid. LCMS: m/z=525.5 [M+H]⁺.

Step 3: Synthesis of 2-amino-4-(5-chloro-1-(4-chloro-7,8-dihydro-5H-pyrano[4,3-d]pyrimidin-2-yl)-3-fluoro-6-methyl-1H-indazol-4-yl)-7-fluorobenzo[b]thiophene-3-carbonitrile

To a solution of the title compound from Step 2 (45.0 g, 72.0 mmol) in toluene (450 mL) was added POCl₃ (117 g, 763 mmol, 71.1 mL) at rt. The mixture was then stirred at 100° C. for 2 h the diluted with water (800 mL) upon cooling to rt under N₂. The pH of the mixture was adjusted to pH 7-8 with the addition of Na2CO₃ solution then extracted with EtOAc (2×1.0 L). The combined organics were washed with brine (2×1.2 L), dried over Na₂SO₄, filtered and concentrated under reduced pressure. The residue was purified by SiO₂ chromatography eluting with PE:EtOAc:DCM=20:1:0 to 5:1:5 to afford the title compound (31.0 g) as a brown solid. LCMS: m/z=543.2 [M+H]⁺.

Step 4: Synthesis of tert-butyl (4-(5-chloro-1-(4-chloro-7,8-dihydro-5H-pyrano[4,3-d]pyrimidin-2-yl)-3-fluoro-6-methyl-1H-indazol-4-yl)-3-cyano-7-fluorobenzo[b]thiophen-2-yl)carbamate

To a solution of the title compound from Step 3 (21.5 g, 39.6 mmol) in DCM (215 mL) at rt was added TEA (8.01 g, 11.0 mL, 79.1 mmol), Boc₂O(10.4 g, 10.9 mL, 47.5 mmol) and DMAP (2.42 g, 19.8 mmol) and the mixture was stirred for 1 hr at rt. The mixture was diluted with water (200 mL) and extracted with DCM (2×150 mL). The combined organics were washed with brine (2×200 mL), dried over Na₂SO₄, filtered, and concentrated under reduced pressure. The residue was purified via SiO₂ chromatography eluting with PE:EtOAc=1:0-PE:EtOAc:DCM=5:1:2 to afford the title compound (18.5 g, 70.0% yield) as a brown solid. LCMS: m/z=643.2 [M+H]⁺.

Step 5: Synthesis of tert-butyl 4-[2-[4-[2-(tert-butoxycarbonylamino)-3-cyano-7-fluoro-benzothiophen-4-yl]-5-chloro-3-fluoro-6-methyl-indazol-1-yl]-7,8-dihydro-5H-pyrano[4,3-d]pyrimidin-4-yl]piperidine-1-carboxylate

A mixture of the title compound fro Step 4 (0.5 g, 777 μmol), tert-butyl 4-bromopiperidine-1-carboxylate (821 mg, 3.11 mmol), bis[3,5-difluoro-2-[5-(trifluoromethyl)-2-pyridyl]phenyl]iridium (1+); 4-tert-butyl-2-(4-tert-butyl-2-pyridyl)pyridine; hexafluorophosphate (8.72 mg, 7.77 μmol), 4-tert-butyl-2-(4-tert-butyl-2-pyridyl)pyridine; dichloronickel (1.55 mg, 3.89 μmol), bis(trimethylsilyl) silyl-trimethyl-silane (193 mg, 777 μmol) and Na₂CO₃ (165 mg, 1.55 mmol) in DME (10 mL) was degassed and purged with N₂, and then the mixture was stirred at rt for 16 h and irradiated with a 455 nm blue LED. The reaction mixture was filtered and concentrated in vacuo. The residue was purified by SiO₂ column chromatography (PE/EtOAc=60/40) to give the title compound (490 mg, 80% yield) as yellow solid. ¹H NMR (400 MHz, CDCl₃) δ=8.80 (s, 1H), 7.88 (s, 1H), 7.31 (dd, 1H), 7.21-7.11 (m, 1H), 4.86 (s, 2H), 4.37 (s, 2H), 4.08 (t, 2H), 3.12 (t, 2H), 2.87 (s, 2H), 2.75 (t, 1H), 2.68-2.60 (m, 3H), 2.00 (s, 2H), 1.85 (s, 2H), 1.53 (d, 18H).

Step 6: Synthesis of 2-amino-4-[5-chloro-3-fluoro-6-methyl-1-[4-(4-piperidyl)-7,8-dihydro-5H-pyrano[4,3-d]pyrimidin-2-yl]indazol-4-yl]-7-fluoro-benzothiophene-3-carbonitrile

To a solution of the title compound from Step 5 (220 mg, 278 μmol) in DCM (3 L) was added TFA (2.30 g, 20.2 mmol) and the mixture was stirred at 30° C. for 0.5 h then concentrated in vacuo to give the title (0.196 g, crude, TFA) as yellow solid. LCMS: m/z=592.2 [M+H]⁺, The crude was used for next step without purification.

Step 7: Synthesis of (S)-2-amino-4-(5-chloro-3-fluoro-6-methyl-1-(4-(1-methylpiperidin-4-yl)-7,8-dihydro-5H-pyrano[4,3-d]pyrimidin-2-yl)-1H-indazol-4-yl)-7-fluorobenzo[b]thiophene-3-carbonitrile and (R)-2-amino-4-(5-chloro-3-fluoro-6-methyl-1-(4-(1-methylpiperidin-4-yl)-7,8-dihydro-5H-pyrano[4,3-d]pyrimidin-2-yl)-1H-indazol-4-yl)-7-fluorobenzo[b]thiophene-3-carbonitrile

To a solution of the title compound from Step 6 (196 mg, 278 μmol, TFA) in MeOH (5 mL) was added TEA (140 mg, 1.39 mmol) and the mixture was stirred at 20° C. for 5 min. AcOH (50.0 mg, 833 μmol) and formaldehyde (113 mg, 1.39 mmol, 37%) was added to the mixture, stirred at 20° C. for 25 min then sodium cyanoborohydride (87.2 mg, 1.39 mmol) was added. The reaction mixture was stirred at 20° C. for 0.5 h then basified with saturated sodium bicarbonate solution (10 mL), diluted with water (20 mL), extracted with EtOAc (3×20 mL). The combined organic layer was washed with brine (40 mL), dried over Na₂SO₄, filtered and concentrated in vacuo. The residue was purified by prep-HPLC (column: WePure PrePulite Platinum C18 150*25 mm*7 μm; mobile phase: [H₂O (0.225% FA)-ACN]; gradient:18%-48% B over 15.0 min) to give a crude. The crude was purified by prep-SFC (column: DAICEL CHIRALPAK AD (250 mm*30 mm, 10 um); mobile phase: [CO₂-EtOH (0.1% NH₃H₂O)]; B %: 50%, isocratic elution mode) to give Peak 1, (18.4 mg) as yellow solid and Peak 2 (10.1 mg) as yellow solid.

Peak 1, (S)-2-amino-4-(5-chloro-3-fluoro-6-methyl-1-(4-(1-methylpiperidin-4-yl)-7,8-dihydro-5H-pyrano[4,3-d]pyrimidin-2-yl)-1H-indazol-4-yl)-7-fluorobenzo[b]thiophene-3-carbonitrile or (R)-2-amino-4-(5-chloro-3-fluoro-6-methyl-1-(4-(1-methylpiperidin-4-yl)-7,8-dihydro-5H-pyrano[4,3-d]pyrimidin-2-yl)-1H-indazol-4-yl)-7-fluorobenzo[b]thiophene-3-carbonitrile

LCMS: m/z=606.3 [M+H]⁺, ¹H NMR (400 MHz, DMSO-d₆) δ 8.75 (s, 1H), 8.05 (s, 2H), 7.32 (dd, 1H), 7.15 (t, 1H), 4.86 (s, 2H), 4.00 (t, 2H), 2.99 (t, 2H), 2.93 (d, 2H), 2.71-2.61 (m, 1H), 2.58 (s, 3H), 2.23 (s, 3H), 2.06-1.86 (m, 4H), 1.84-1.73 (m, 2H)

Peak 2, (R)-2-amino-4-(5-chloro-3-fluoro-6-methyl-1-(4-(1-methylpiperidin-4-yl)-7,8-dihydro-5H-pyrano[4,3-d]pyrimidin-2-yl)-1H-indazol-4-yl)-7-fluorobenzo[b]thiophene-3-carbonitrile or (S)-2-amino-4-(5-chloro-3-fluoro-6-methyl-1-(4-(1-methylpiperidin-4-yl)-7,8-dihydro-5H-pyrano[4,3-d]pyrimidin-2-yl)-1H-indazol-4-yl)-7-fluorobenzo[b]thiophene-3-carbonitrile

LCMS: m/z=606.3 [M+H]⁺, ¹H NMR (400 MHz, DMSO-d₆) δ 8.74 (s, 1H), 8.05 (s, 2H), 7.32 (dd, 1H), 7.15 (t, 1H), 4.86 (s, 2H), 4.01 (t, 2H), 3.09-2.90 (m, 4H), 2.76-2.65 (m, 1H), 2.58 (s, 3H), 2.30 (s, 3H), 2.15 (t, 2H), 2.03-1.89 (m, 2H), 1.83 (d, 2H)

### Example 172

### (S)-2-amino-4-(5-chloro-3-fluoro-6-methyl-1-(4-((4-methylpiperazin-1-yl)methyl)-7,8-dihydro-5H-pyrano[4,3-d]pyrimidin-2-yl)-1H-indazol-4-yl)-7-fluorobenzo[b]thiophene-3-carbonitrile or (R)-2-amino-4-(5-chloro-3-fluoro-6-methyl-1-(4-((4-methylpiperazin-1-yl)methyl)-7,8-dihydro-5H-pyrano[4,3-d]pyrimidin-2-yl)-1H-indazol-4-yl)-7-fluorobenzo[b]thiophene-3-carbonitrile

### And Example 173

### (R)-2-amino-4-(5-chloro-3-fluoro-6-methyl-1-(4-((4-methylpiperazin-1-yl)methyl)-7,8-dihydro-5H-pyrano[4,3-d]pyrimidin-2-yl)-1H-indazol-4-yl)-7-fluorobenzo[b]thiophene-3-carbonitrile or (S)-2-amino-4-(5-chloro-3-fluoro-6-methyl-1-(4-((4-methylpiperazin-1-yl)methyl)-7,8-dihydro-5H-pyrano[4,3-d]pyrimidin-2-yl)-1H-indazol-4-yl)-7-fluorobenzo[b]thiophene-3-carbonitrile

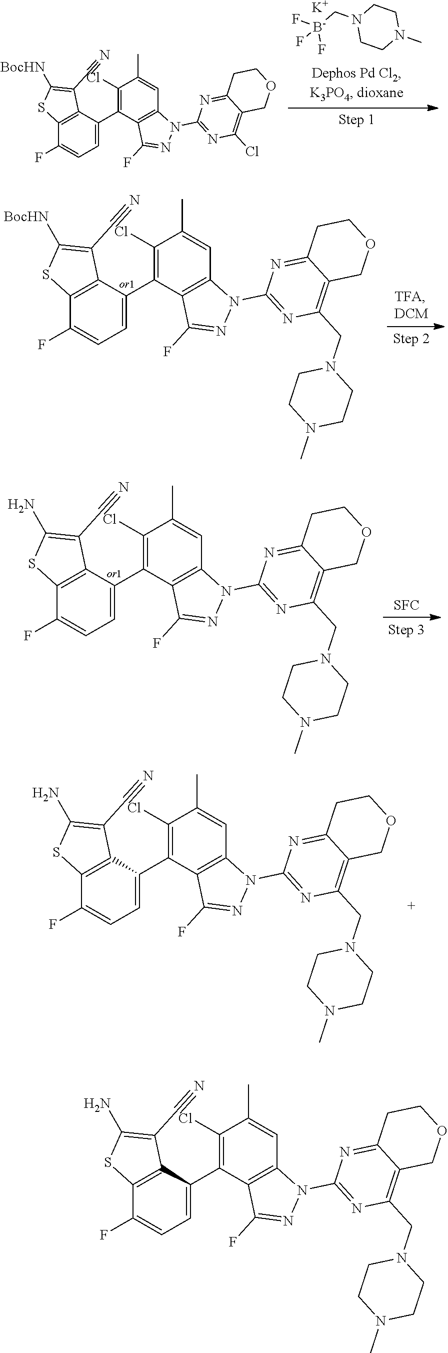

Step 1: Synthesis of tert-butyl (4-(5-chloro-3-fluoro-6-methyl-1-(4-((4-methylpiperazin-1-yl)methyl)-7,8-dihydro-5H-pyrano[4,3-d]pyrimidin-2-yl)-1H-indazol-4-yl)-3-cyano-7-fluorobenzo[b]thiophen-2-yl)carbamate

A mixture of the title compound from Example 170 and 171, Step 4 (700 mg, 1.09 mmol), Potassium trifluoro ((4-methylpiperazin-1-yl)methyl) borate (718.2 mg, 3.26 mmol), Dephos Pd Cl₂ (77.9 mg, 108.7 μmol), K₃PO₄ (692.7 mg, 3.26 mmol) in dioxane (10 mL) was degassed and purged with N₂ for 3 times, and then the mixture was stirred at 100° C. for 16 h under N₂. The mixture was diluted with water 10 mL and extracted with EtOAc 60 mL (20 mL×3). The combined organic layers were washed with water 30 mL (10 mL*3), dried over anhydrous Na₂SO₄, filtered and concentrated under reduced pressure. The residue was purified by reversed-phase HPLC([water (FA)-ACN]; gradient:10%-100% B, 15 min) to give compound the title compound (300 mg, 34.03% yield) as white solid, LCMS: m/z=721.2 [M+H]⁺.

Step 2: Synthesis of 2-amino-4-(5-chloro-3-fluoro-6-methyl-1-(4-((4-methylpiperazin-1-yl)methyl)-7,8-dihydro-5H-pyrano[4,3-d]pyrimidin-2-yl)-1H-indazol-4-yl)-7-fluorobenzo[b]thiophene-3-carbonitrile

A mixture of the title compound from Step 1 (100 mg, 138.6 μmol), TFA (767.5 mg, 6.7 mmol, 0.5 mL) in DCM (1 mL) was stirred at 25° C. for 10 min, the mixture was concentrated and the residue was purified by reversed-phase flash([water (FA)-ACN]; gradient:0%-70% B, 15 min) to give the title compound (60 mg, 69.67% yield) as off-white solid, LCMS: m/z=621.2 [M+H]⁺.

Step 3: Synthesis of (S)-2-amino-4-(5-chloro-3-fluoro-6-methyl-1-(4-((4-methylpiperazin-1-yl)methyl)-7,8-dihydro-5H-pyrano[4,3-d]pyrimidin-2-yl)-1H-indazol-4-yl)-7-fluorobenzo[b]thiophene-3-carbonitrile and (R)-2-amino-4-(5-chloro-3-fluoro-6-methyl-1-(4-((4-methylpiperazin-1-yl)methyl)-7,8-dihydro-5H-pyrano[4,3-d]pyrimidin-2-yl)-1H-indazol-4-yl)-7-fluorobenzo[b]thiophene-3-carbonitrile

The title compound from Step 2 (60 mg, 96.60 μmol) was separated by chiral SFC (column: DAICEL CHIRALCEL OD (250 mm*30 mm, 10 um); mobile phase: [CO2-IPA:ACN=4:1 (0.1% NH3H2O)]; B %: 37%, isocratic elution mode) to give Peak 1, (S)-2-amino-4-(5-chloro-3-fluoro-6-methyl-1-(4-((4-methylpiperazin-1-yl)methyl)-7,8-dihydro-5H-pyrano[4,3-d]pyrimidin-2-yl)-1H-indazol-4-yl)-7-fluorobenzo[b]thiophene-3-carbonitrile or (R)-2-amino-4-(5-chloro-3-fluoro-6-methyl-1-(4-((4-methylpiperazin-1-yl)methyl)-7,8-dihydro-5H-pyrano[4,3-d]pyrimidin-2-yl)-1H-indazol-4-yl)-7-fluorobenzo[b]thiophene-3-carbonitrile, (20.1 mg, 32.66% yield) as yellow solid and Peak 2 (R)-2-amino-4-(5-chloro-3-fluoro-6-methyl-1-(4-((4-methylpiperazin-1-yl)methyl)-7,8-dihydro-5H-pyrano[4,3-d]pyrimidin-2-yl)-1H-indazol-4-yl)-7-fluorobenzo[b]thiophene-3-carbonitrile or (S)-2-amino-4-(5-chloro-3-fluoro-6-methyl-1-(4-((4-methylpiperazin-1-yl)methyl)-7,8-dihydro-5H-pyrano[4,3-d]pyrimidin-2-yl)-1H-indazol-4-yl)-7-fluorobenzo[b]thiophene-3-carbonitrile (18.9 mg, 30.38% yield) as a yellow solid

Peak 1: LCMS data: M+1 (621.2); 1H NMR (400 MHz, 6d-DMSO): δ ppm 8.80 (s, 1H), 8.05 (s, 2H), 7.30 (dd, 1H), 7.23-7.09 (m, 1H), 4.89 (s, 2H), 4.03 (t, 2H), 3.73 (s, 2H), 3.04 (t, 2H), 2.95-2.70 (m, 4H), 2.67 (d, 2H), 2.62 (s, 3H), 2.59-2.51 (m, 4H), 2.48-2.47 (m, 1H);

Peak 2: LCMS data: M+1 (621.2); 1H NMR (400 MHz, 6d-DMSO): δ ppm 8.81 (s, 1H), 8.05 (s, 2H), 7.30 (dd, 1H), 7.15 (t, 1H), 4.89 (s, 2H), 4.03 (t, 2H), 3.71 (s, 2H), 3.03 (t, 2H), 2.66 (s, 3H), 2.62 (s, 3H), 2.60-2.53 (m, 4H), 2.44-2.23 (m, 4H);

### Biological Examples

### Example B1. Nucleotide Exchange Assay (NEA)

### Materials and Reagents

Buffer composition: 10 mM HEPES pH 7.4, 150 mM NaCl, 0.05% BSA, 5 mM MgCl2, 0.0025% IGEPAL prepared from HEPES, 1.0 M solution, pH 7.2-7.4 (H1095, Solarbio), NaCl 5.0 M solution (71386-1L, Sigma), BSA (ST023-50g, Beyotime), MgCl₂ 1.0 M solution (7786-30-3, Sigma) and Igepal (9002-93-1, Sigma).

Reagents: GST-tagged KRAS mutants (Biortus); GST-tagged HRAS Wt (Biortus, HRas-H02C-GDP); GST-tagged NRAS Wt (Biortus, NRas-H02I-GDP); SOS1 (220782-WDF220803-BCS, BCS); EDA-GTP-DY-647P1 (NU-820-647P1, Jena Bioscience); anti-GST-Tb

Final concentration of the reagents in the assay: reagent protein: 5 nM; SOS1: 20 nM; EDA-GTP-DY-647P1: 10 nM; anti-GST-Tb: 1 nM

Plate: Proxiplate-384 plus (6008260, PerkinElmer)

| Reagent | Source | Catalog |
|---|---|---|
| HEPES, 1.0M solution, | Solarbio | H1095 |
| pH 7.2-7.4 | | |
| NaCl 5.0M solution | SIGMA | 71386-1L |
| BSA | Beyotime | ST023-50g |
| Igepal | SIGMA | 9002-93-1 |
| anti-GST-Tb | Cisbio | 61GSTTLB |
| MgCl2 1.0 M solution | SIGMA | 7786-30-3 |
| EDA-GTP-DY-647P1 | Jena Bioscience | NU-820-647P1 |
| 384-well Proxiplate-384 plus | PerkinElmer | 6008260 |
| GST-tagged KRAS G12D | Biortus | KRas4B-H02K-GDP |
| GST-tagged KRAS G12V | Biortus | KRas4B-H02I-GDP |
| GST-tagged KRAS G12C | Biortus | KRas4B-H02M-GDP |
| GST-tagged KRAS G12A | Biortus | KRas4B-H02Q-GDP |
| GST-tagged KRAS G12R | Biortus | BP18375-2DA |
| GST-tagged KRAS G13D | Biortus | KRas4B-H02L-GDP |
| GST-tagged KRAS Wt | Biortus | KRas4B-H02O-GDP |
| GST-tagged HRAS Wt | Biortus | HRas-H02C-GDP |
| GST-tagged NRAS Wt | Biortus | NRas-H02I-GDP |
| SOS1 | BCS | 220782-WDF220803- |
| | | BCS |

### Procedure

After distributing 100 nL of the compounds (in DMSO) with serial 2-fold dilutions from 100 μM to the assay plate by Echo 550 (Labcyte), 5 μL of a mixture of GST-tagged KRAS and anti-GST-Tb were delivered to the plate by multidrop (ThermoFisher Scientific). The plate was centrifuged (Cence) at 1000 rpm for 1 min and pre-incubated for 10 minutes at RT. After the pre-incubation, 5 μL of a mixture of SOS1 and EDA-GTP-DY-647P1 was added to the plate by multidrop. After centrifugation at 1000 rpm for 1 min and incubation for 60 minutes at RT, the plate was read in Neo2 (Bio Tek) at 665 nm and 620 nm. Results are shown in Table 2.

TABLE 2

| | NEA | NEA | NEA | NEA | NEA | NEA | NEA | NEA | NEA |
|---|---|---|---|---|---|---|---|---|---|
| | KRAS | KRAS | KRAS | KRAS | KRAS | KRAS | KRAS | HRAS | NRAS |
| | G12D | G12V | G12C | G12A | G12R | G13D | WT | WT | WT |
| Example | IC50 | IC50 | IC50 | IC50 | IC50 | IC50 | IC50 | IC50 | IC50 |
| No. | (nM) | (nM) | (nM) | (nM) | (nM) | (nM) | (nM) | (nM) | (nM) |
| 1 | 2008.45 | 584.50 | ND | ND | ND | ND | ND | ND | ND |
| 2 | 2.77 | 1.51 | 1.67 | ND | 1.66 | ND | ND | 592.35 | 490.13 |
| 3 | 2.23 | 1.33 | 0.93 | 1.16 | 0.93 | 1.41 | 1.41 | 76.59 | 208.87 |
| 4 | 313.82 | 200.94 | ND | ND | ND | ND | ND | ND | ND |
| 5 | 35.27 | 24.08 | 34.26 | 44.09 | 26.91 | 67.44 | 55.79 | 1339.95 | 2337.95 |
| 6 | 3.38 | 2.74 | 3.17 | 3.21 | 1.86 | 3.01 | 2.43 | 10.87 | 27.06 |
| 7 | 227.36 | 86.88 | ND | ND | ND | ND | ND | ND | ND |
| 8 | 3.17 | 1.64 | 2.86 | 3.01 | 2.08 | 7.08 | 4.41 | 421.16 | 698.05 |
| 9 | 167.58 | 157.89 | ND | ND | ND | ND | ND | ND | ND |
| 10 | 2.50 | 1.82 | 1.76 | 2.09 | 1.49 | 2.20 | 2.22 | 52.13 | 165.16 |
| 11 | 62.65 | 36.84 | 47.20 | 85.69 | 56.23 | 174.13 | 103.15 | 4543.72 | 6854.20 |
| 12 | 1.99 | 1.72 | 2.38 | 2.64 | 1.54 | 3.85 | 3.21 | 13.95 | 33.17 |
| 13 | 37.74 | 30.07 | 30.05 | 43.54 | 27.96 | 165.78 | 32.11 | 7342.18 | 8589.27 |
| 14 | 2.50 | 1.69 | 2.16 | 2.35 | 1.50 | 2.33 | 2.22 | 30.71 | 82.60 |
| 15 | 2.93 | 2.11 | 3.10 | 3.62 | 1.75 | 5.14 | 4.29 | 190.04 | 395.36 |
| 16 | 60.69 | 54.28 | 49.65 | 89.87 | 54.10 | 191.50 | 122.02 | 12068.99 | 13464.31 |
| 17 | 218.16 | 489.87 | ND | ND | ND | ND | ND | ND | ND |
| 18 | 2.09 | 1.73 | 2.45 | 2.81 | 1.80 | 4.16 | 1.30 | 121.89 | 430.40 |
| 19 | 3.05 | 2.07 | 2.43 | 3.21 | 1.81 | 6.09 | 3.83 | 263.05 | 551.76 |
| 20 | 176.60 | 158.85 | ND | ND | ND | ND | ND | ND | ND |
| 21 | 447.36 | 203.01 | ND | ND | ND | ND | ND | ND | ND |
| 22 | 2.07 | 1.58 | ND | ND | ND | ND | ND | 115.67 | 288.26 |
| 23 | 227.50 | 116.43 | ND | ND | ND | ND | ND | ND | ND |
| 24 | 2.14 | 1.30 | 1.89 | 2.17 | 1.56 | 2.43 | 2.44 | 50.87 | 117.02 |
| 25 | 5762.49 | 1859.79 | ND | ND | ND | ND | ND | ND | ND |
| 26 | 2.57 | 1.67 | 3.01 | 2.83 | 1.96 | 5.12 | 2.06 | 455.44 | 1075.73 |
| 27 | 648.39 | 506.85 | ND | ND | ND | ND | ND | ND | ND |
| 28 | 1.61 | 1.33 | 1.74 | 1.89 | 1.31 | 2.14 | 1.95 | 8.49 | 20.11 |
| 29 | 11754.13 | 2492.64 | ND | ND | ND | ND | ND | ND | ND |
| 30 | 29.53 | 2.71 | 5.48 | ND | 7.80 | ND | ND | 2557.06 | 2179.58 |
| 31 | 4427.38 | 547.49 | ND | ND | ND | ND | ND | ND | ND |
| 32 | 36.59 | 3.09 | 7.16 | ND | 7.75 | ND | ND | 2054.74 | 2162.30 |
| 33 | 2983.53 | 804.26 | ND | ND | ND | ND | ND | ND | ND |
| 34 | 16.73 | 2.89 | 5.35 | 12.58 | 7.55 | 2251.91 | 12.15 | 4701.16 | 3305.21 |
| 35 | 2407.38 | 294.23 | ND | ND | ND | ND | ND | ND | ND |
| 36 | 5.99 | 2.11 | 2.47 | 4.46 | 3.38 | 2154.15 | 5.83 | 1763.39 | 1855.11 |
| 37 | 24.34 | 2.65 | 8.04 | 21.15 | 11.29 | 222.51 | 34.26 | 20194.10 | 20654.78 |
| 38 | 4402.84 | 1291.61 | ND | ND | ND | ND | ND | ND | ND |
| 39 | 2.87 | 1.30 | 1.85 | ND | 2.20 | ND | ND | 1060.10 | 1891.55 |
| 40 | 49.81 | 4.00 | 9.91 | 29.81 | 16.60 | 12154.01 | 44.27 | 52822.78 | 43193.22 |
| 41 | 30.86 | 4.04 | 8.99 | 22.83 | 13.11 | 64822.19 | 28.96 | 35347.79 | 36598.78 |
| 42 | 30.03 | 4.83 | 8.68 | 22.36 | 12.92 | 12023.65 | 34.08 | 26072.09 | 24666.90 |
| 43 | 3.52 | 1.72 | 2.23 | 2.72 | 1.82 | 3.60 | 2.96 | 343.23 | 537.81 |
| 44 | 2555.70 | 863.16 | ND | ND | ND | ND | ND | ND | ND |
| 45 | 3.30 | 1.41 | 1.82 | ND | 1.70 | ND | ND | 297.05 | 330.67 |
| 46 | 21.87 | 3.98 | 6.55 | ND | 12.86 | ND | ND | 12931.93 | 14407.48 |
| 47 | 18104.37 | 2278.90 | ND | ND | ND | ND | ND | ND | ND |
| 48 | 19.60 | 2.33 | 5.52 | 9.62 | 6.54 | 60363.19 | 19.62 | 11485.81 | 9609.54 |
| 49 | 15622.05 | 3934.70 | ND | ND | ND | ND | ND | ND | ND |
| 50 | 27.00 | 4.17 | 11.43 | 27.95 | 14.56 | 15822.86 | 31.02 | 14899.69 | 15986.85 |
| 51 | 4004.88 | 320.19 | ND | ND | ND | ND | ND | ND | ND |
| 52 | 15.25 | 1.72 | 3.41 | 7.80 | 3.08 | 21325.77 | 11.39 | 8342.48 | 6546.81 |
| 53 | 6330.07 | 2781.21 | ND | ND | ND | ND | ND | ND | ND |
| 54 | 4.91 | 1.56 | 2.36 | 4.41 | 2.16 | 4105.81 | 5.20 | 3392.97 | 3705.83 |
| 55 | 13399.95 | 3737.48 | ND | ND | ND | ND | ND | ND | ND |
| 56 | 25.31 | 6.84 | 19.97 | 57.51 | 37.10 | 78518.82 | 55.64 | 34359.59 | 35523.02 |
| 57 | 6944.32 | 2256.92 | ND | ND | ND | ND | ND | ND | ND |
| 58 | 8.22 | 1.94 | 5.20 | 12.18 | 4.88 | 69.06 | 11.85 | 2454.14 | 2923.97 |
| 59 | 9462.90 | 1516.09 | ND | ND | ND | ND | ND | ND | ND |
| 60 | 20.73 | 3.52 | 5.06 | 7.28 | 5.40 | 4042.32 | 11.70 | 7819.89 | 8549.68 |
| 61 | 8.57 | 2.49 | 3.13 | 5.61 | 3.07 | 3583.52 | 7.16 | 402.78 | 527.22 |
| 62 | 1946.84 | 581.95 | ND | ND | ND | ND | ND | ND | ND |
| 63 | 11.33 | 1.58 | 4.69 | 9.60 | 5.21 | 5648.59 | 11.26 | 5708.36 | 3402.74 |
| 64 | 1298.67 | 980.91 | ND | ND | ND | ND | ND | ND | ND |
| 65 | 2.35 | 1.84 | ND | ND | ND | ND | ND | 481.73 | 904.31 |
| 66 | 15408.86 | 4634.06 | ND | ND | ND | ND | ND | ND | ND |
| 67 | 10.00 | 2.09 | 2.59 | 3.52 | 2.45 | 3774.49 | 4.50 | 1915.07 | 1669.58 |
| 68 | 3118.27 | 1179.89 | ND | ND | ND | ND | ND | ND | ND |
| 69 | 5.00 | 2.37 | 3.72 | 2.19 | 2.20 | 7.24 | 1.53 | 408.39 | 621.74 |
| 70 | 12618.46 | 2751.70 | ND | ND | ND | ND | ND | ND | ND |
| 71 | 25.88 | 2.23 | 5.41 | 12.55 | 5.33 | 969.53 | 22.16 | 9988.64 | 8980.68 |
| 72 | 65100.86 | 100000.00 | ND | ND | ND | ND | ND | ND | ND |
| 73 | 47.11 | 4.80 | 8.05 | ND | 12.50 | ND | ND | 7902.62 | 6300.81 |
| 74 | 16.61 | 2.15 | 5.28 | 13.99 | 7.69 | 207.95 | 19.83 | 14340.36 | 12991.67 |
| 75 | 474.27 | 181.01 | ND | ND | ND | ND | ND | ND | ND |
| 76 | 3.08 | 1.42 | 2.39 | 2.90 | 1.39 | 5.15 | 4.33 | 508.96 | 1048.55 |
| 77 | 920.03 | 241.82 | ND | ND | ND | ND | ND | ND | ND |
| 78 | 3.40 | 1.93 | 3.26 | 3.45 | 1.60 | 9.14 | 2.30 | 511.86 | 606.18 |
| 79 | 1656.78 | 609.18 | ND | ND | ND | ND | ND | ND | ND |
| 80 | 2.60 | 1.50 | 1.55 | 2.01 | 1.46 | 2.13 | 1.76 | 74.82 | 329.51 |
| 81 | 19.39 | 2.99 | 7.89 | 18.00 | 9.52 | 13410.82 | 25.65 | 12319.99 | 13343.34 |
| 82 | 23.20 | 2.49 | 6.80 | 16.99 | 8.73 | 2982.22 | 27.51 | 24209.30 | 26727.57 |
| 83 | 48.40 | 22.47 | 31.34 | 58.08 | 30.30 | 1995.82 | 38.00 | 19981.04 | 14768.60 |
| 84 | 10029.28 | 2330.10 | ND | ND | ND | ND | ND | ND | ND |
| 85 | 286.16 | 59.46 | ND | ND | ND | ND | ND | ND | ND |
| 86 | 2.51 | 1.29 | 2.05 | 2.05 | 1.41 | 2.88 | 2.82 | 88.15 | 171.45 |
| 87 | 1452.48 | 367.33 | ND | ND | ND | ND | ND | ND | ND |
| 88 | 7.08 | 1.88 | 3.13 | 3.64 | 2.04 | 21.37 | 4.47 | 709.78 | 1503.21 |
| 89 | 11262.92 | 2962.51 | ND | ND | ND | ND | ND | ND | ND |
| 90 | 25.00 | 4.30 | 4.63 | 9.42 | 7.53 | 16240.06 | 14.39 | 5130.22 | 4243.04 |
| 91 | 8774.14 | 1059.69 | ND | ND | ND | ND | ND | ND | ND |
| 92 | 29.47 | 3.27 | 8.63 | 23.44 | 12.57 | 22859.61 | 29.73 | 11648.10 | 13000.89 |
| 93 | 6200.33 | 949.24 | ND | ND | ND | ND | ND | ND | ND |
| 94 | 21.65 | 4.02 | 7.95 | 21.81 | 11.25 | 23242.38 | 20.66 | 9068.20 | 7425.46 |
| 95 | 776.97 | 192.16 | ND | ND | ND | ND | ND | ND | ND |
| 96 | 2.92 | 1.12 | 2.16 | 2.70 | 1.73 | 5.58 | 4.67 | 315.30 | 192.83 |
| 97 | 18943.49 | 3799.53 | ND | ND | ND | ND | ND | ND | ND |
| 98 | 74.15 | 10.48 | 14.93 | 22.45 | 12.32 | 15375.97 | 33.29 | 11072.77 | 15746.40 |
| 99 | 1514.03 | 801.86 | ND | ND | ND | ND | ND | ND | ND |
| 100 | 3.46 | 2.02 | 2.61 | 3.26 | 1.70 | 3.78 | 4.07 | 308.92 | 514.75 |
| 101 | 6831.45 | 1358.01 | ND | ND | ND | ND | ND | ND | ND |
| 102 | 17.59 | 3.13 | 5.03 | 11.48 | 6.30 | 8119.44 | 11.63 | 8787.06 | 8180.83 |
| 103 | 8011.20 | 3533.30 | ND | ND | ND | ND | ND | ND | ND |
| 104 | 161.64 | 15.43 | ND | ND | ND | ND | ND | ND | ND |
| 105 | 170.06 | 19.55 | ND | ND | ND | ND | ND | ND | ND |
| 106 | 515.65 | 44.36 | ND | ND | ND | ND | ND | ND | ND |
| 107 | 265.04 | 29.23 | ND | ND | ND | ND | ND | ND | ND |
| 108 | 635.09 | 48.97 | ND | ND | ND | ND | ND | ND | ND |
| 109 | 736.87 | 56.57 | ND | ND | ND | ND | ND | ND | ND |
| 110 | 715.47 | 49.33 | ND | ND | ND | ND | ND | ND | ND |
| 111 | 454.92 | 50.73 | ND | ND | ND | ND | ND | ND | ND |
| 112 | 276.27 | 28.57 | ND | ND | ND | ND | ND | ND | ND |
| 113 | 2.17 | 1.86 | 1.62 | 1.94 | 1.5 | 2.1 | 2.00 | 6.1 | 13.6 |
| 114 | 3.51 | 2.19 | 1.79 | 1.96 | 1.9 | 2.4 | 2.57 | 7.9 | 27.8 |
| 115 | 3.41 | 2.21 | 2.09 | 2.12 | 1.9 | 2.3 | 2.40 | 21.7 | 66.5 |
| 28a | 1.58 | 1.28 | 1.74 | 1.89 | 1.4 | 2.1 | 1.95 | 8.5 | 20.1 |
| 12a | 1.99 | 1.72 | 2.38 | 2.64 | 1.3 | 3.8 | 3.21 | 14.0 | 33.2 |
| 117 | 7.71 | 2.09 | 2.85 | 2.33 | 2.3 | 3.4 | 3.21 | 136.0 | 311.3 |
| 118 | 1.95 | 1.66 | 1.48 | 1.58 | 1.3 | 1.8 | 1.63 | 20.6 | 43.9 |
| 119 | 2.04 | 1.70 | 1.41 | 1.54 | 1.1 | 1.7 | 1.76 | 12.9 | 27.4 |
| 120 | 3.87 | 2.41 | 1.79 | 1.89 | 1.6 | 2.3 | 2.64 | 8.4 | 24.0 |
| 121 | 2.44 | 1.82 | 1.52 | 1.65 | 1.3 | 2.0 | 2.25 | 7.0 | 15.4 |
| 122 | 1.94 | 1.67 | 1.69 | 1.74 | 1.3 | 2.0 | 1.81 | 18.4 | 55.9 |
| 123 | 1.95 | 1.60 | 1.60 | 1.78 | 1.1 | 2.2 | 1.80 | 5.6 | 14.0 |
| 124 | 2.25 | 1.72 | 1.77 | 1.78 | 1.6 | 2.0 | 1.83 | 15.9 | 69.0 |
| 125 | 1.37 | 0.79 | 0.68 | 0.75 | 0.6 | 0.9 | 0.92 | 16.9 | 59.3 |
| 126 | 2.17 | 1.59 | 1.38 | 1.62 | 1.3 | 1.8 | 1.77 | 27.0 | 73.0 |
| 127 | 4.93 | 2.35 | 1.38 | 2.67 | 2.0 | 3.2 | 3.51 | 65.3 | 227.7 |
| 128 | 37.74 | 26.98 | 27.46 | 31.32 | 21.9 | 47.8 | 31.53 | 1108.9 | 2355.1 |
| 129 | 2.19 | 1.47 | 1.81 | 1.93 | 1.8 | 2.1 | 1.99 | 16.6 | 55.4 |
| 130 | 91.08 | 64.69 | 31.05 | 31.11 | 28.8 | 35.7 | 32.91 | 456.4 | 722.7 |
| 131 | 1.91 | 1.61 | 1.24 | ND | 1.2 | 1.3 | 1.50 | 3.8 | 5.3 |
| 132 | 93.51 | 49.62 | 54.45 | 69.04 | 51.5 | 73.3 | 65.74 | 518.5 | 1103.1 |
| 133 | 2.23 | 1.81 | 1.77 | 1.75 | 1.5 | 2.2 | 1.90 | 6.4 | 14.9 |
| 134 | 276.34 | 191.32 | 163.22 | 213.45 | 150.0 | 233.2 | 284.47 | 2551.9 | 6975.4 |
| 135 | 1.80 | 1.68 | 1.13 | ND | 1.4 | 2.3 | 2.36 | 18.0 | 51.6 |
| 136 | 532.26 | 416.02 | 326.83 | 374.74 | 277.5 | 469.7 | 446.49 | 2485.1 | 5888.9 |
| 137 | 2.22 | 1.93 | 1.60 | 1.73 | 1.5 | 1.8 | 1.87 | 15.5 | 55.0 |
| 138 | 689.48 | 449.74 | 346.14 | 536.04 | 408.0 | 1037.4 | 636.34 | 3184.7 | 6680.1 |
| 139 | 1.80 | 1.80 | 1.65 | 1.68 | 1.3 | 1.8 | 1.91 | 8.7 | 23.3 |
| 140 | 49.01 | 32.32 | 30.43 | 34.03 | 27.7 | 38.9 | 39.66 | 866.6 | 1684.5 |
| 141 | 2.95 | 2.15 | 1.95 | 1.99 | 1.7 | 2.8 | 2.13 | 7.0 | 13.4 |
| 142 | 128.22 | 100.15 | 90.90 | 102.52 | 76.6 | 110.1 | 110.25 | 400.2 | 891.0 |
| 143 | 2.11 | 1.58 | 1.40 | 1.43 | 1.1 | 1.6 | 1.76 | 2.6 | 3.2 |
| 144 | 1930.76 | 817.77 | 846.35 | 1143.23 | 806.8 | 1915.6 | 1640.43 | 14523.0 | 24634.7 |
| 145 | 1.65 | 1.80 | 1.86 | 1.89 | 1.7 | 2.7 | 1.99 | 23.2 | 109.1 |
| 146 | 1658.99 | 703.53 | 740.63 | 1165.95 | 487.0 | 4567.0 | 1727.99 | 59700.1 | 54538.9 |
| 147 | 4.18 | 1.51 | 1.17 | 1.57 | 1.2 | 1.8 | 1.95 | 39.5 | 191.8 |
| 148 | 515.85 | 316.68 | 296.39 | 322.56 | 247.9 | 431.5 | 426.94 | 6880.5 | 9005.8 |
| 149 | 2.57 | 1.85 | 1.93 | 1.85 | 1.8 | 2.5 | 2.37 | 24.1 | 68.7 |
| 150 | 87.64 | 43.05 | 40.23 | 47.74 | 36.5 | 79.9 | 60.88 | 2840.5 | 4935.5 |
| 151 | 2.46 | 1.87 | 1.88 | 1.89 | 1.6 | 2.3 | 2.02 | 36.1 | 107.2 |
| 152 | 194.33 | 101.58 | 118.08 | 148.14 | 117.1 | 330.9 | 216.45 | 728.0 | 792.0 |
| 153 | 2.71 | 2.36 | 1.44 | 1.66 | 1.4 | 1.9 | 1.88 | 55.5 | 141.5 |
| 154 | 463.83 | 277.20 | 923.96 | 1178.89 | 841.8 | 1417.9 | 1302.68 | 6501.4 | 12069.2 |
| 155 | 2.09 | 1.49 | 1.94 | 1.93 | 1.7 | 2.6 | 2.05 | 35.3 | 82.0 |
| 156 | 470.65 | 155.07 | 169.90 | 345.28 | 236.1 | 1148.5 | 458.89 | 16263.6 | 34062.1 |
| 157 | 2.71 | 1.81 | 1.69 | 1.93 | 1.5 | 2.7 | 2.09 | 78.5 | 272.3 |
| 158 | 119.97 | 60.11 | 42.96 | 82.75 | 57.3 | 173.3 | 111.54 | 3077.8 | 7474.2 |
| 159 | 2.22 | 1.39 | 0.93 | 1.53 | 1.0 | 1.6 | 1.61 | 46.3 | 147.1 |
| 160 | 1443.40 | 972.42 | 840.96 | 1045.56 | 741.0 | 1546.2 | 1407.92 | 24189.2 | 97279.4 |
| 161 | 2.07 | 1.47 | 1.46 | 1.87 | 1.6 | 3.1 | 2.18 | 90.1 | 281.4 |
| 162 | 256.6 | 184.0 | 134.9 | 196.3 | 143.7 | 294.4 | 315.6 | 4,545.2 | 3,631.2 |
| 163 | 2.3 | 1.9 | 1.85 | 2.0 | 1.7 | 3.8 | 3.10 | 53.9 | 63.8 |
| 164 | 8.4 | 6.6 | 6.9 | 14.7 | 14.2 | 150.0 | 19.1 | 25,115.4 | 12,637.3 |
| 165 | 70,403.2 | 75,729.6 | 60,827.1 | 92,274.5 | 100,000.0 | 48,516.0 | 70,238.9 | 100,000.0 | 62,899.9 |
| 166 | 327.7 | 202.9 | 220.2 | 375.7 | 180.4 | 363.3 | 370.4 | 1122.4 | 2195.2 |
| 167 | 1.8 | 1.6 | 1.4 | 1.9 | 1.5 | 2.3 | 2.0 | 21.7 | 79.7 |
| 168 | 184.2 | 123.2 | 133.1 | 220.1 | 126.3 | 335.2 | 200.9 | 2402.8 | 4997.2 |
| 169 | 1.7 | 1.6 | 1.4 | 2.0 | 1.4 | 2.4 | 1.9 | 21.1 | 72.5 |
| 170 | 1266.3 | 506.3 | 618.7 | 1211.0 | 801.0 | 3703.1 | 1395.5 | 24173.8 | 24689.8 |
| 171 | 6.6 | 2.6 | 3.2 | 7.0 | 3.8 | 34.1 | 7.4 | 503.4 | 1060.0 |
| 172 | 12.4 | 3.4 | 5.5 | 13.7 | 6.9 | 75.1 | 15.4 | 996.8 | 1604.7 |
| 173 | 1070.7 | 261.5 | 401.1 | 888.2 | 441.9 | 4639.3 | 1052.8 | 35758.1 | 31487.6 |

ND = not determined

The foregoing disclosure has been described in some detail by way of illustration and example, for purposes of clarity and understanding. Therefore, it is to be understood that the above description is intended to be illustrative and not restrictive. The scope of the disclosure should, therefore, be determined not with reference to the above description, but should instead be determined with reference to the following appended claims, along with the full scope of equivalents to which such claims are entitled.

1. A compound of Formula (I): 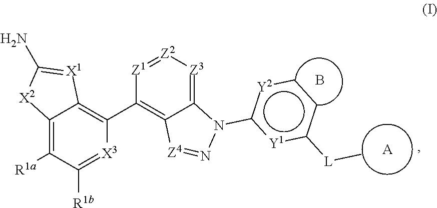
or a pharmaceutically acceptable salt thereof, wherein:
X¹ is N or C—CN;
X² is S or Se;
X³ is N or CR^{1c},
Z¹ is CR^{a} or N, Z² is CR^{b} or N; and Z³ is CR^{c} or N, provided that only one of Z¹, Z², and Z³ is N;
Z⁴ is CR^{d} or N;
Y¹ is CR^{e} or N;
Y² is CR^{f} or N;
wherein at least one of Y¹ and Y² is N;
L is selected from a bond, —(CR^{La}R^{Lb})ₙ—, —(CR^{La}R^{Lb})ₘ₁O(CR^{La}R^{Lb})ₘ₂—, and —(CR^{La}R^{Lb})ₘ₁NR^{L}(CR^{La}R^{Lb})ₘ₂—;
R^{L} is H or C₁-C₄ alkyl;
R^{La} and R^{Lb} are each independently selected from H, D, and C₁-C₄ alkyl, wherein the alkyl is optionally substituted with 1 to 5 groups independently selected from D and halo;
n is selected from 1, 2, and 3;
m1 and m2 are each independently selected from 0, 1, 2, and 3, wherein m1+m2 is ≤4;
Ring A is a C₃-C₈ cycloalkyl, phenyl, 3 to 10 membered heterocyclyl ring, or 5 to 10 membered heteroaryl ring, wherein the cycloalkyl, phenyl, heterocyclyl, or heteroaryl ring are optionally substituted with 1 to 5 groups independently selected from D, halo, ═O, OR^{g}, NR^{g}R^{g}, C(O)NR^{g}R^{g}, —CN, C₁-C₄ alkyl, and C₂-C₄ alkenyl, and wherein the alkyl and alkenyl are optionally substituted with 1 to 5 groups independently selected from D, halo, ═O, OR^{g}, NR^{g}R^{g}, C(O)NR^{g}R^{g}, CN, and 3 to 8 membered heterocyclyl ring optionally substituted with 1 to 5 groups independently selected from C₁-C₄ alkyl, wherein the heterocyclyl or heteroaryl ring comprises 1 or 2 heteroatoms selected from N, NR^{g}, O, and S;
Ring B is a C₃-C₁₂ cycloalkyl or 4 to 14 membered heterocyclyl, wherein the cycloalkyl or heterocyclyl are optionally substituted with 1 to 5 groups independently selected from D, halo, ═O, OR^{g}, NR^{g}R^{g}, C(O)NR^{g}R^{g}, —CN, C₁-C₄ alkyl, and C₂-C₄ alkenyl, wherein the alkyl and alkenyl are optionally substituted with 1 to 5 groups independently selected from D, halo, ═O, OR^{g}, NR^{g}R^{g}, C(O)NR^{g}R^{g}, and CN, wherein the heterocyclyl comprises 1 or 2 heteroatoms selected from N, NR^{g}, O, and S; or
R^{1a}, R^{1b}, and R^{1c} are each independently selected from H, D, halo, and C₁-C₄ alkyl, wherein the alkyl is optionally substituted with 1 to 3 groups independently selected from D and halo;
R^{a}, R^{b}, R^{c} and R^{d} are each independently selected from H, D, halo, CN, OR^{g}, N(R^{g})₂, C₁-C₄ alkyl, C₃-C₆ cycloalkyl, 5 to 6 membered heterocyclyl, and 5 or 6 membered heteroaryl, wherein the alkyl is optionally substituted with 1 to 5 groups independently selected from D, halo, ═O, OR^{g}, N(R^{g})₂C(O)NR^{g}R^{g}, 5 to 6 membered heterocyclyl and 5 to 6 membered heteroaryl, and wherein the heteroaryl and heterocyclyl comprises 1-3 heteroatoms selected from NR^{g}, O, and S and are optionally substituted with 1 to 5 groups independently selected from D, halo, ═O, C₁-C₄ alkyl, and C₁-C₄ alkoxy, wherein the alkyl and alkoxy are optionally substituted with 1 to 5 groups independently selected from D, halo, and OR^{g}, and wherein the cycloalkyl is optionally substituted with 1 to 5 groups independently selected from D, halo, ═O, C₁-C₄ alkyl, and C₁-C₄ alkoxy, wherein the alkyl and alkoxy are optionally substituted with 1 to 5 groups independently selected from D, halo, and OR^{g}; or
R^{a} and R^{b}, together with the carbon atoms to which they are attached, form a cyclopentyl, cyclohexyl, or a 5 to 6 membered heterocyclyl, wherein the cyclopentyl, cyclohexyl, or 5 to 6 membered heterocyclyl are optionally substituted with 1 to 3 groups independently selected from D, halo, C₁-C₄ alkyl, and C₁-C₄ alkoxy, wherein the alkyl and alkoxy are optionally substituted with 1 to 5 groups independently selected from D, halo, and OR^{g}, and the heterocyclyl comprises 1 or 2 heteroatoms selected from N, NR^{g}, O, and S; or
R^{b} and R^{c}, together with the carbon atoms to which they are attached, form a cyclopentyl, cyclohexyl, or a 5 to 6 membered heterocyclyl, wherein the cyclopentyl, cyclohexyl, or 5 to 6 membered heterocyclyl are optionally substituted with 1 to 3 groups independently selected from D, halo, C₁-C₄ alkyl, and C₁-C₄ alkoxy, wherein the alkyl and alkoxy are optionally substituted with 1 to 5 groups independently selected from D, halo, and OR^{g}, and the heterocyclyl comprises 1 or 2 heteroatoms selected from N, NR^{g}, O, and S;
R^{e} and R^{f} are each selected from H, halo, and C₁-C₄ alkyl, wherein the alkyl is optionally substituted with 1 to 5 groups independently selected from D and halo; and
R^{g} is each independently selected from H and C₁-C₄ alkyl, wherein the alkyl is optionally substituted with 1 to 5 groups independently selected from D and halo.

2. A compound of claim 1, wherein the compound is of Formula (II): 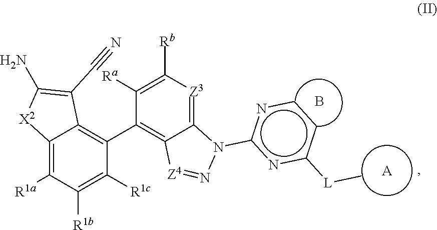
or a pharmaceutically acceptable salt thereof, wherein:
X² is S or Se;
Z³ is CR^{c} or N;
Z⁴ is CR^{d} or N;
L is selected from a bond and O(CR^{La}R^{Lb})ₘ₂—;
R^{La} and R^{Lb} are each independently selected from H, D, and C₁-C₄ alkyl, wherein the alkyl is optionally substituted with 1 to 5 groups independently selected from D and halo;
m2 is independently selected from 0, 1, and 2;
Ring A is a C₃-C₈ cycloalkyl or 3 to 10 membered heterocyclyl ring, wherein the cycloalkyl or heterocyclyl are optionally substituted with 1 to 5 groups independently selected from D, halo, ═O, OR^{g}, NR^{g}R^{g}, and C₁-C₄ alkyl, and wherein the alkyl is optionally substituted with 1 to 5 groups independently selected from D and halo, wherein the heterocyclyl ring comprises 1 or 2 heteroatoms selected from N, NR^{g}, O, and S;
Ring B is a C₃-C₈ cycloalkyl or 5 to 12 membered heterocyclyl, wherein the cycloalkyl or heterocyclyl are optionally substituted with 1 to 5 groups independently selected from D, halo, ═O, OR^{g}, and C₁-C₄ alkyl, wherein the alkyl is optionally substituted with 1 to 5 groups independently selected from D, halo, ═O, OR^{g}, and NR^{g}R^{g}, wherein the heterocyclyl comprises 1 or 2 heteroatoms selected from N, NR^{g}, O, and S; or
R^{1a}, R^{1b}, and R^{1c} are each independently selected from H, D, halo, and C₁-C₄ alkyl;
R^{a}, R^{b}, R^{c} and R^{d} are each independently selected from H, D, halo, OR^{g}, N(R^{g})₂, C₁-C₄ alkyl, and C₃-C₆ cycloalkyl, wherein the alkyl is optionally substituted with 1 to 5 groups independently selected from D, halo, and OR^{g}, and wherein the cycloalkyl is optionally substituted with 1 to 5 groups independently selected from halo, ═O, C₁-C₄ alkyl, and C₁-C₄ alkoxy, wherein the alkyl and alkoxy are optionally substituted with 1 to 5 groups independently selected from halo and OR^{g}; or
R^{a} and R^{b}, together with the carbon atoms to which they are attached, form a cyclopentyl, cyclohexyl, or a 5 to 6 membered heterocyclyl, wherein the heterocyclyl comprises 1 or 2 oxygen atoms; or
R^{b} and R^{c}, together with the carbon atoms to which they are attached, form a cyclopentyl, cyclohexyl, or a 5 to 6 membered heterocyclyl, wherein the heterocyclyl comprises 1 or 2 oxygen atoms; and
R^{g} is each independently selected from H and C₁-C₄ alkyl, wherein the alkyl is optionally substituted with 1 to 5 groups independently selected from D and halo.

3. A compound of claim 1, wherein the compound is of Formula (III): 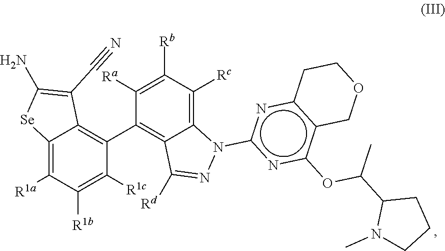
or a pharmaceutically acceptable salt thereof, wherein:
R^{1a}, R^{1b}, and R^{1c} are each independently selected from H, halo, and C₁-C₄ alkyl;
R^{a}, R^{b}, R^{c} and R^{d} are each independently selected from H, D, halo, OR^{g}, N(R^{g})₂, C₁-C₄ alkyl, and C₃-C₆ cycloalkyl, wherein the alkyl is optionally substituted with 1 to 5 groups independently selected from D, halo, and OR^{g}, and wherein the cycloalkyl is optionally substituted with 1 to 5 groups independently selected from halo, ═O, C₁-C₄ alkyl, and C₁-C₄ alkoxy, wherein the alkyl and alkoxy are optionally substituted with 1 to 5 groups independently selected from halo and OR^{g}; or
R^{g} is each independently selected from H and C₁-C₄ alkyl, wherein the alkyl is optionally substituted with 1 to 5 groups independently selected from D and halo.

4. The compound of claim 1, wherein the compound is of Formula (IVa), (IVb), (IVc), (IVd), or (IVf): 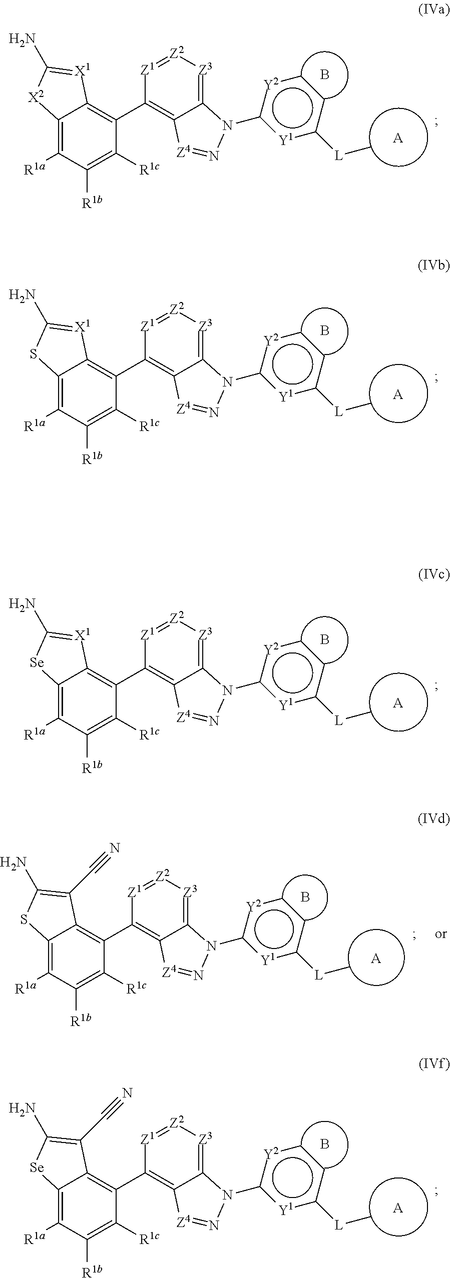
or a pharmaceutically acceptable salt thereof.

5. The compound of claim 1, wherein;
R^{1a} and R^{1c} are each independently selected from H, halo, and C₁-C₄ alkyl;
R^{1b} is H;
Z¹ is CR^{a}, Z² is CR^{b}; and Z³ is CR^{c} or N;
Z⁴ is CR^{d} or N;
Y¹ is N;
Y² is N;
L is selected from a bond and O(CR^{La}R^{Lb})ₘ₂—;
R^{La} and R^{Lb} are each independently selected from H, D, and C₁-C₄ alkyl, wherein the alkyl is optionally substituted with 1 to 4 groups independently selected from D and halo;
m2 is selected from 0, 1, and 2;
Ring A is a C₃-C₈ cycloalkyl or 3 to 10 membered heterocyclyl ring, wherein the cycloalkyl or heterocyclyl ring are optionally substituted with 1 to 4 groups independently selected from D, halo, ═O, OR^{g}, N(R^{g})₂, and C₁-C₄ alkyl, and wherein the alkyl is optionally substituted with 1 to 4 groups independently selected from D and halo, wherein the heterocyclyl ring comprises 1 or 2 heteroatoms selected from N, NR^{g}, and O;
Ring B is a C₃-C₈ cycloalkyl or 5 to 12 membered heterocyclyl, wherein the cycloalkyl or heterocyclyl are optionally substituted with 1 to 4 groups independently selected from D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂, wherein the heterocyclyl comprises 1 or 2 heteroatoms selected from N, NR^{g}, and O; or
R^{a}, R^{b}, R^{c} and R^{d} are each independently selected from H, D, halo, OR^{g}, N(R^{g})₂, and C₁-C₄ alkyl, wherein the alkyl is optionally substituted with 1 to 4 groups independently selected from D, halo, ═O, OR^{g}, and N(R^{g})₂; or
R^{a} and R^{b}, together with the carbon atoms to which they are attached, form a cyclopentyl, cyclohexyl, or a 5 to 6 membered heterocyclyl, wherein the cyclopentyl, cyclohexyl, or 5 to 6 membered heterocyclyl are optionally substituted with 1 to 3 groups independently selected from D, halo, C₁-C₄ alkyl, and C₁-C₄ alkoxy, wherein the alkyl and alkoxy are optionally substituted with 1 to 3 groups independently selected from D, halo and OR^{g}, and the heterocyclyl comprises 1 or 2 heteroatoms selected from NR^{g} and O; or
R^{b} and R^{c}, together with the carbon atoms to which they are attached, form a cyclopentyl, cyclohexyl, or a 5 to 6 membered heterocyclyl, wherein the cyclopentyl, cyclohexyl, or 5 to 6 membered heterocyclyl are optionally substituted with 1 to 3 groups independently selected from D, halo, C₁-C₄ alkyl, and C₁-C₄ alkoxy, wherein the alkyl and alkoxy are optionally substituted with 1 to 3 groups independently selected from D, halo, and OR^{g}, and the heterocyclyl comprises 1 or 2 heteroatoms selected from NR^{g} and O; and
R^{g} is each independently selected from H and C₁-C₄ alkyl, wherein the alkyl is optionally substituted with 1 to 4 groups independently selected from D and halo.

6. (canceled)

7. The compound of claim 1, wherein the compound is of Formula (Va): 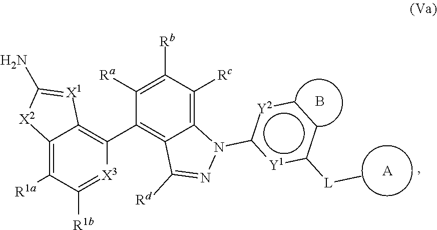
or a pharmaceutically acceptable salt thereof, wherein:
X¹ is N or C—CN;
X² is S or Se;
X³ is N or CR^{1c};
Y¹ is N;
Y² is N;
L is selected from a bond and O(CR^{La}R^{Lb})ₘ₂—;
R^{La} and R^{Lb} are each independently selected from H, D, and C₁-C₄ alkyl, wherein the alkyl is optionally substituted with 1 to 4 groups independently selected from D and halo;
m2 is selected from 0, 1, and 2;
Ring A is a C₃-C₈ cycloalkyl or 3 to 10 membered heterocyclyl ring, wherein the cycloalkyl or heterocyclyl ring are optionally substituted with 1 to 4 groups independently selected from D, halo, ═O, OR^{g}, N(R^{g})₂, and C₁-C₄ alkyl, and wherein the alkyl is optionally substituted with 1 to 4 groups independently selected from D and halo, wherein the heterocyclyl ring comprises 1 or 2 heteroatoms selected from N, NR^{g}, and O;
Ring B is a C₃-C₈ cycloalkyl or 5 to 12 membered heterocyclyl, wherein the cycloalkyl or heterocyclyl are optionally substituted with 1 to 4 groups independently selected from D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂, wherein the heterocyclyl comprises 1 or 2 heteroatoms selected from N, NR^{g}, and O; or
R^{1a} and R^{1c} are each independently selected from H, halo, and C₁-C₄ alkyl and R^{1b} is H;
R^{a}, R^{b}, R^{c} and R^{d} are each independently selected from H, D, halo, OR^{g}, N(R^{g})₂, and C₁-C₄ alkyl, wherein the alkyl is optionally substituted with 1 to 4 groups independently selected from D, halo, ═O, OR^{g}, and N(R^{g})₂; or
R^{a} and R^{b}, together with the carbon atoms to which they are attached, form a cyclopentyl, cyclohexyl, or a 5 to 6 membered heterocyclyl, wherein the cyclopentyl, cyclohexyl, or 5 to 6 membered heterocyclyl are optionally substituted with 1 to 3 groups independently selected from D, halo, C₁-C₄ alkyl, and C₁-C₄ alkoxy, wherein the alkyl and alkoxy are optionally substituted with 1 to 3 groups independently selected from D, halo, and OR^{g}, and the heterocyclyl comprises 1 or 2 heteroatoms selected from NR and O; or
R^{b} and R^{c}, together with the carbon atoms to which they are attached, form a cyclopentyl, cyclohexyl, or a 5 to 6 membered heterocyclyl, wherein the cyclopentyl, cyclohexyl, or 5 to 6 membered heterocyclyl are optionally substituted with 1 to 3 groups independently selected from D, halo, C₁-C₄ alkyl, and C₁-C₄ alkoxy, wherein the alkyl and alkoxy are optionally substituted with 1 to 3 groups independently selected from D, halo, and OR^{g}, and the heterocyclyl comprises 1 or 2 heteroatoms selected from NR^{g} and O; and
R^{g} is each independently selected from H and C₁-C₄ alkyl, wherein the alkyl is optionally substituted with 1 to 4 groups independently selected from D and halo.

8. (canceled)

9. (canceled)

10. The compound of claim 1, wherein the compound is of Formula (VIa): 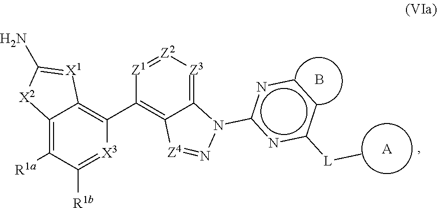
or a pharmaceutically acceptable salt thereof, wherein:
Ring B is a C₃-C₈ cycloalkyl or 5 to 12 membered heterocyclyl, wherein the cycloalkyl or heterocyclyl are optionally substituted with 1 to 4 groups independently selected from D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂, wherein the heterocyclyl comprises 1 or 2 heteroatoms selected from N, NR^{g}, and O.

11. The compound of claim 1, wherein the compound is of Formula (VIb), (VIk), or (VIu): 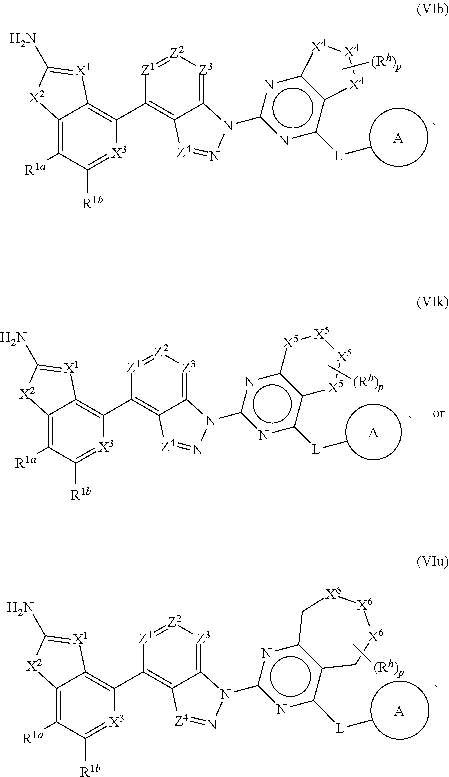
or a pharmaceutically acceptable salt thereof, wherein:
X⁴ is each independently selected from O, NR^{g}, and C(R^{h})₂, provided that at least one X⁴ is C(R^{h})₂;
X⁵ is each independently selected from O, NR^{g}, and C(R^{h})₂, provided that at least two X⁵ are C(R^{h})₂;
X⁶ is each independently selected from O, NR^{g}, or C(R^{h})₂, provided that at least two X⁶ are C(R^{h})₂;
R^{h} is each independently selected from H, D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂; or
2 R^{h}, along with the carbon atom to which they are attached, form a 4 to 6 membered heterocyclyl; and
p is selected from 0, 1, 2, 3, and 4.

12. The compound of claim 1, wherein the compound is of Formula (VIc) or (VII): 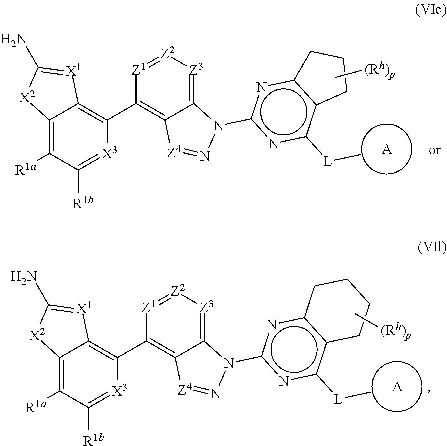
or a pharmaceutically acceptable salt thereof, wherein:
R^{h} is H, D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂; and
p is selected from 0, 1, 2, 3, and 4.

13. The compound of claim 1, wherein the compound is of Formula (VId) or (VIe): 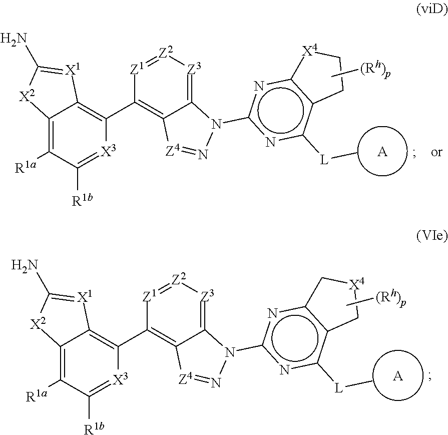
or a pharmaceutically acceptable salt thereof, wherein:
X⁴ is selected from O and NR^{g};
R^{h} is each independently selected from H, D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂; and
p is selected from 0, 1, 2, 3, and 4.

14. (canceled)

15. (canceled)

16. The compound of claim 1, wherein the compound is of Formula (VIm), (VIn), (VIo), or (VIr): 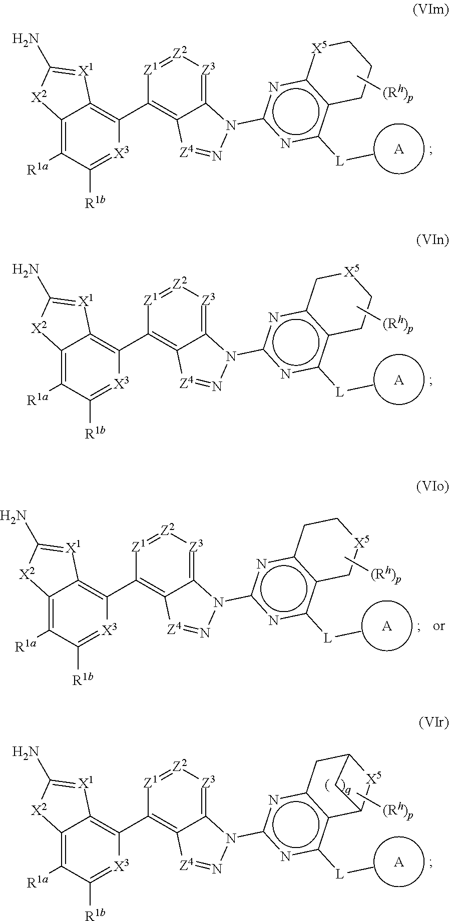
or a pharmaceutically acceptable salt thereof, wherein:
X⁵ is selected from O and NR^{g};
R^{h} is each independently selected from H, D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂;
p is selected from 0, 1, 2, 3, and 4; and
q is selected from 1 and 2.

17. The compound of claim 1, wherein the compound is of Formula (VIq): 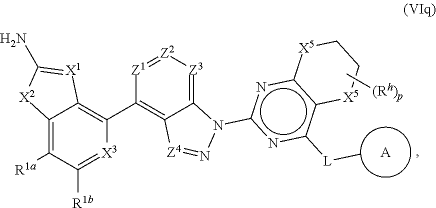
or a pharmaceutically acceptable salt thereof, wherein:
X⁵ is each independently selected from O and NR^{g};
R^{h} is each independently selected from H, D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂; and
p is selected from 0, 1, 2, 3, and 4.

18. (canceled)

19. (canceled)

20. The compound of claim 1, wherein the compound is of Formula (VIw): 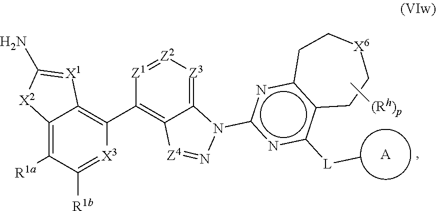
or a pharmaceutically acceptable salt thereof, wherein:
X⁶ is selected from O and NR^{g};
R^{h} is each independently selected from D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂; and
p is selected from 0, 1, 2, 3, and 4.

21. The compound of claim 1, wherein the compound is of Formula (VIy), (VIz), (VIaa), (VIab), (VIad), (VIah), (VIal), or (VIam): 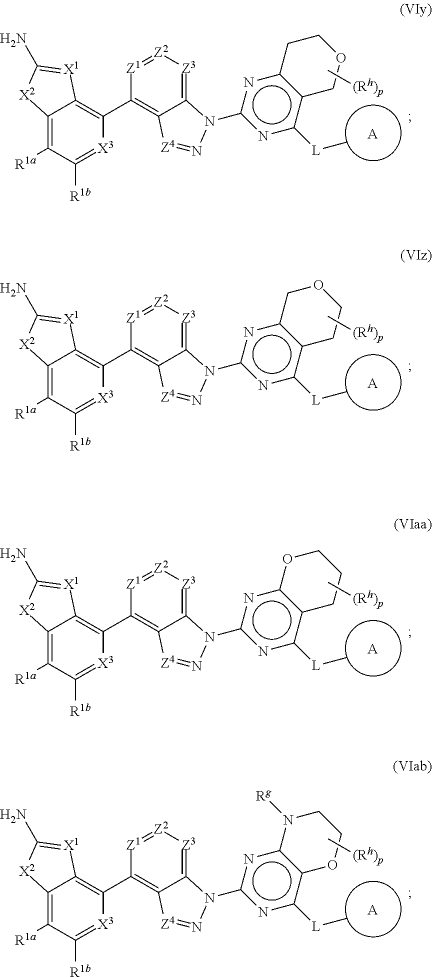 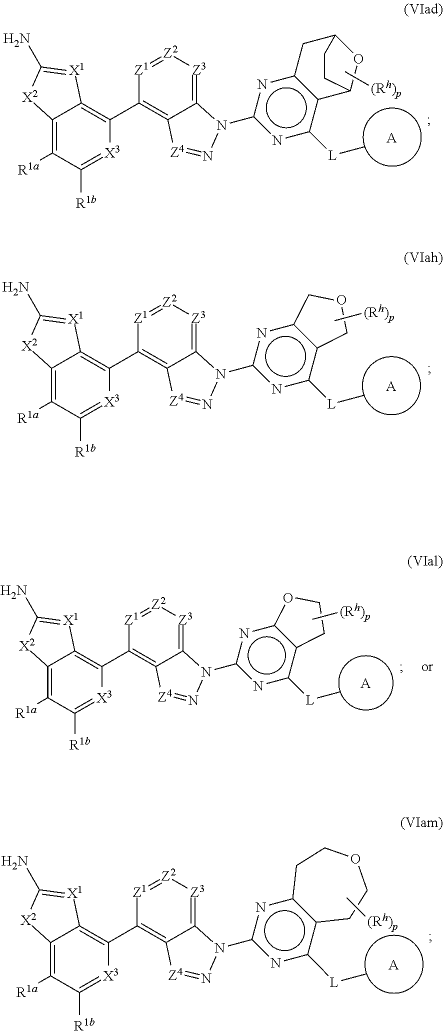
or a pharmaceutically acceptable salt thereof, wherein:
R^{h} is each independently selected from D, halo, ═O, OR^{g}, N(R^{g})₂, and C₁-C₄ alkyl, wherein the alkyl is optionally substituted with 1 to 4 groups independently selected from D, halo, ═O, OR^{g}, and N(R^{g})₂; and
p is selected from 0, 1, 2, 3, and 4.

22. The compound of claim 10, wherein
X¹ is N or C—CN;
X² is S or Se;
X³ is N or CR^{1c};
Z¹ is CR^{a}, Z² is CR^{b}; and Z³ is CR^{c} or N;
Z⁴ is CR^{d} or N;
L is selected from a bond and O(CR^{La}R^{Lb})ₘ₂—;
R^{La} and R^{Lb} are each independently selected from H, D, and C₁-C₄ alkyl, wherein the alkyl is optionally substituted with 1 to 4 groups independently selected from D and halo;
m2 is selected from 0, 1, and 2;
Ring A is a C₃-C₈ cycloalkyl or 3 to 10 membered heterocyclyl ring, wherein the cycloalkyl or heterocyclyl ring are optionally substituted with 1 to 4 groups independently selected from D, halo, ═O, OR^{g}, N(R^{g})₂, and C₁-C₄ alkyl, and wherein the alkyl is optionally substituted with 1 to 4 groups independently selected from D and halo, wherein the heterocyclyl ring comprises 1 or 2 heteroatoms selected from N, NR^{g}, and O;
R^{1a} and R^{1c} are each independently selected from H, halo, and C₁-C₄ alkyl and R^{1b} is H;
R^{a}, R^{b}, R^{c} and R^{d} are each independently selected from H, D, halo, OR^{g}, N(R^{g})₂, and C₁-C₄ alkyl, wherein the alkyl is optionally substituted with 1 to 4 groups independently selected from D, halo, ═O, OR^{g}, and N(R^{g})₂; or
R^{a} and R^{b}, together with the carbon atoms to which they are attached, form a cyclopentyl, cyclohexyl, or a 5 to 6 membered heterocyclyl, wherein the cyclopentyl, cyclohexyl, or 5 to 6 membered heterocyclyl are optionally substituted with 1 to 3 groups independently selected from D, halo, C₁-C₄ alkyl, and C₁-C₄ alkoxy, wherein the alkyl and alkoxy are optionally substituted with 1 to 3 groups independently selected from D, halo, and OR^{g}, and the heterocyclyl comprises 1 or 2 heteroatoms selected from NR^{g} and O; or
R^{b} and R^{c}, together with the carbon atoms to which they are attached, form a cyclopentyl, cyclohexyl, or a 5 to 6 membered heterocyclyl, wherein the cyclopentyl, cyclohexyl, or 5 to 6 membered heterocyclyl are optionally substituted with 1 to 3 groups independently selected from D, halo, C₁-C₄ alkyl, and C₁-C₄ alkoxy, wherein the alkyl and alkoxy are optionally substituted with 1 to 3 groups independently selected from D, halo, and OR^{g}, and the heterocyclyl comprises 1 or 2 heteroatoms selected from NR^{g} and O; and
R^{g} is each independently selected from H and C₁-C₄ alkyl, wherein the alkyl is optionally substituted with 1 to 4 groups independently selected from D and halo.

23.-50. (canceled)

51. A compound selected from Table 1, or a pharmaceutically acceptable salt thereof.

52. The compound of claim 1, or a pharmaceutically acceptable salt thereof, wherein:
X¹ is C—CN;
X² is S or Se;
X³ is N or CR^{1c};
Z¹ is CR^{a}, Z² is CR^{b} or N; and Z³ is CR^{c};
Z⁴ is CR^{d} or N;
Y¹ is N;
Y² is N;
L is selected from a bond and —(CR^{La}R^{Lb})ₘ₁O(CR^{La}R^{Lb})ₘ₂—;
R^{La} and R^{Lb} are each independently selected from H and C₁-C₄ alkyl, wherein the alkyl is optionally substituted with 1 to 3 groups independently selected from D and halo;
m1 and m2 are each independently selected from 0 and 1;
Ring A is a C₃-C₈ cycloalkyl or 3 to 10 membered heterocyclyl ring, wherein the cycloalkyl or heterocyclyl ring are optionally substituted with 1 to 3 groups independently selected from D, halo, ═O, OR^{g}, NR^{g}R^{g}, C(O)NR^{g}R^{g}, —CN, C₁-C₄ alkyl, and C₂-C₄ alkenyl, and wherein the alkyl and alkenyl are optionally substituted with 1 to 3 groups independently selected from D, halo, ═O, OR^{g}, NR^{g}R^{g}, C(O)NR^{g}R^{g}, CN, and 3 to 8 membered heterocyclyl ring optionally substituted with 1 to 3 groups independently selected from C₁-C₄ alkyl, wherein the heterocyclyl or heteroaryl ring comprises 1 or 2 heteroatoms selected from N, NR^{g}, O, and S;
Ring B is a C₃-C₁₂ cycloalkyl or 4 to 14 membered heterocyclyl, wherein the cycloalkyl or heterocyclyl are optionally substituted with 1 to 3 groups independently selected from D, halo, ═O, OR^{g}, NR^{g}R^{g}, C(O)NR^{g}R^{g}, —CN, C₁-C₄ alkyl, and C₂-C₄ alkenyl, wherein the alkyl and alkenyl are optionally substituted with 1 to 3 groups independently selected from D, halo, ═O, OR^{g}, NR^{g}R^{g}, C(O)NR^{g}R^{g}, and CN, wherein the heterocyclyl comprises 1 or 2 heteroatoms selected from N, NR^{g}, O, and S; or
R^{1a}, R^{1b}, and R^{1c} are each independently selected from H and halo;
R^{a}, R^{b}, R^{c} and R^{d} are each independently selected from H, halo, OR^{g}, N(R^{g})₂, and C₁-C₄ alkyl, wherein the alkyl is optionally substituted with 1 to 3 groups independently selected from D, halo, ═O, OR^{g}, N(R^{g})₂C(O)NR^{g}R^{g}, 5 to 6 membered heterocyclyl and 5 to 6 membered heteroaryl, and wherein the heteroaryl and heterocyclyl comprises 1-3 heteroatoms selected from NR^{g}, O, and S and are optionally substituted with 1 to 3 groups independently selected from D, halo, ═O, C₁-C₄ alkyl, and C₁-C₄ alkoxy, wherein the alkyl and alkoxy are optionally substituted with 1 to 3 groups independently selected from D, halo, and OR^{g}; or
R^{a} and R^{b}, together with the carbon atoms to which they are attached, form a 5 to 6 membered heterocyclyl optionally substituted with 1 to 2 groups independently selected from D, halo, C₁-C₄ alkyl, and C₁-C₄ alkoxy, wherein the alkyl and alkoxy are optionally substituted with 1 to 3 groups independently selected from D, halo, and OR^{g}, and the heterocyclyl comprises 1 or 2 heteroatoms selected from N, NR^{g}, O, and S; and
R^{g} is each independently selected from H and C₁-C₄ alkyl, wherein the alkyl is optionally substituted with 1 to 3 groups independently selected from D and halo.

53. (canceled)

54. The compound of claim 1, wherein the compound is of Formula (I-a) or Formula (I-b): 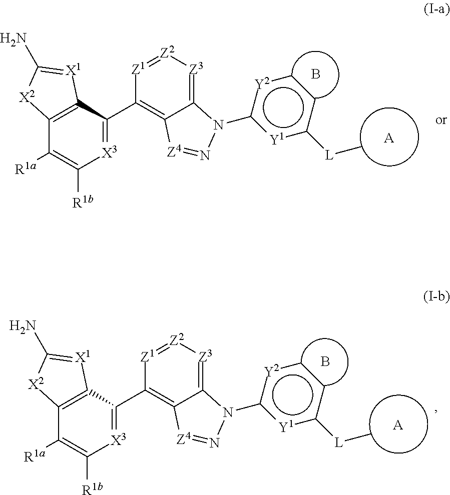
or a pharmaceutically acceptable salt thereof.

55. (canceled)

56. (canceled)

57. (canceled)

58. (canceled)

59. A pharmaceutical composition comprising a compound or pharmaceutically acceptable salt of claim 1 and at least one pharmaceutically acceptable excipient.

60. A method of treating a disease or disorder mediated by KRAS, comprising providing to a subject in need thereof a compound of Formula (I): 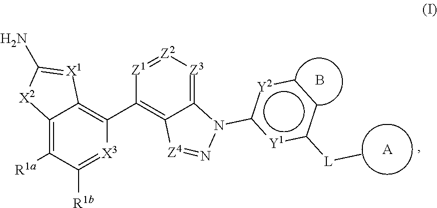
or a pharmaceutically acceptable salt thereof, wherein:
X¹ is N or C—CN;
X² is S or Se;
X³ is N or CR^{1c};
Z¹ is CR^{a} or N, Z² is CR^{b} or N; and Z³ is CR^{c} or N, provided that only one of Z¹, Z², and Z³ is N;
Z⁴ is CR^{d} or N;
Y¹ is CR^{e} or N;
Y² is CR^{f} or N;
wherein at least one of Y¹ and Y² is N;
L is selected from a bond, —(CR^{La}R^{Lb})ₙ—, —(CR^{La}R^{Lb})ₘ₁O(CR^{La}R^{Lb})ₘ₂—, and —(CR^{La}R^{Lb})ₘ₁NR^{L}(CR La R^{Lb})ₘ₂—;
R^{L} is H or C₁-C₄ alkyl;
R^{La} and R^{Lb} are each independently selected from H, D, and C₁-C₄ alkyl, wherein the alkyl is optionally substituted with 1 to 5 groups independently selected from D and halo;
n is selected from 1, 2, and 3;
m1 and m2 are each independently selected from 0, 1, 2, and 3, wherein m1+m2 is ≤4;
Ring A is a C₃-C₈ cycloalkyl, phenyl, 3 to 10 membered heterocyclyl ring, or 5 to 10 membered heteroaryl ring, wherein the cycloalkyl, phenyl, heterocyclyl, or heteroaryl ring are optionally substituted with 1 to 5 groups independently selected from D, halo, ═O, OR^{g}, NR^{g}R^{g} C(O)NR^{g}R^{g}, —CN, C₁-C₄ alkyl, and C₂-C₄ alkenyl, and wherein the alkyl and alkenyl are optionally substituted with 1 to 5 groups independently selected from D, halo, ═O, OR^{g}, NR^{g}R^{g}C(O)NR^{g}R^{g}, CN, and 3 to 8 membered heterocyclyl ring optionally substituted with 1 to 5 groups independently selected from C₁-C₄ alkyl, wherein the heterocyclyl or heteroaryl ring comprises 1 or 2 heteroatoms selected from N, NR^{g}, O, and S;
Ring B is a C₃-C₁₂ cycloalkyl or 4 to 14 membered heterocyclyl, wherein the cycloalkyl or heterocyclyl are optionally substituted with 1 to 5 groups independently selected from D, halo, ═O, OR^{g}, NR^{g}R^{g}, C(O)NR^{g}R^{g}, —CN, C₁-C₄ alkyl, and C₂-C₄ alkenyl, wherein the alkyl and alkenyl are optionally substituted with 1 to 5 groups independently selected from D, halo, ═O, OR^{g}, NR^{g}R^{g}, C(O)NR^{g}R^{g}, and CN, wherein the heterocyclyl comprises 1 or 2 heteroatoms selected from N, NR^{g}, O, and S; or
R^{1a}, R^{1b}, and R^{1c} are each independently selected from H, D, halo, and C₁-C₄ alkyl, wherein the alkyl is optionally substituted with 1 to 3 groups independently selected from D and halo;
R^{a}, R^{b}, R^{c} and R^{d} are each independently selected from H, D, halo, CN, OR^{g}, N(R^{g})₂, C₁-C₄ alkyl, C₃-C₆ cycloalkyl, 5 to 6 membered heterocyclyl, and 5 or 6 membered heteroaryl, wherein the alkyl is optionally substituted with 1 to 5 groups independently selected from D, halo, ═O, OR^{g}, N(R^{g})₂C(O)NR^{g}R^{g}, 5 to 6 membered heterocyclyl and 5 to 6 membered heteroaryl, and wherein the heteroaryl and heterocyclyl comprises 1-3 heteroatoms selected from NR^{g}, O, and S and are optionally substituted with 1 to 5 groups independently selected from D, halo, ═O, C₁-C₄ alkyl, and C₁-C₄ alkoxy, wherein the alkyl and alkoxy are optionally substituted with 1 to 5 groups independently selected from D, halo, and OR^{g}, and wherein the cycloalkyl is optionally substituted with 1 to 5 groups independently selected from D, halo, ═O, C₁-C₄ alkyl, and C₁-C₄ alkoxy, wherein the alkyl and alkoxy are optionally substituted with 1 to 5 groups independently selected from D, halo, and OR^{g}; or
R^{a} and R^{b}, together with the carbon atoms to which they are attached, form a cyclopentyl, cyclohexyl, or a 5 to 6 membered heterocyclyl, wherein the cyclopentyl, cyclohexyl, or 5 to 6 membered heterocyclyl are optionally substituted with 1 to 3 groups independently selected from D, halo, C₁-C₄ alkyl, and C₁-C₄ alkoxy, wherein the alkyl and alkoxy are optionally substituted with 1 to 5 groups independently selected from D, halo, and OR^{g}, and the heterocyclyl comprises 1 or 2 heteroatoms selected from N, NR^{g}, O, and S; or
R^{b} and R^{c}, together with the carbon atoms to which they are attached, form a cyclopentyl, cyclohexyl, or a 5 to 6 membered heterocyclyl, wherein the cyclopentyl, cyclohexyl, or 5 to 6 membered heterocyclyl are optionally substituted with 1 to 3 groups independently selected from D, halo, C₁-C₄ alkyl, and C₁-C₄ alkoxy, wherein the alkyl and alkoxy are optionally substituted with 1 to 5 groups independently selected from D, halo, and OR^{g}, and the heterocyclyl comprises 1 or 2 heteroatoms selected from N, NR^{g}, O, and S;
R^{e} and R^{f} are each selected from H, halo, and C₁-C₄ alkyl, wherein the alkyl is optionally substituted with 1 to 5 groups independently selected from D and halo; and
R^{g} is each independently selected from H and C₁-C₄ alkyl, wherein the alkyl is optionally substituted with 1 to 5 groups independently selected from D and halo.

61. (canceled)

62. (canceled)

63. (canceled)

64. (canceled)

65. The method of claim 60, wherein the KRAS is KRAS wild type (KRAS^{WT}).

66. The method of claim 65, wherein the KRAS^{WT} is amplified, overexpressed, or activated.

67. The method of claim 60, wherein the KRAS comprises a mutation.

68. The method of claim 60, wherein the disease or disorder is mediated by two or more types of KRAS variants.

69. The method of claim 67, wherein the KRAS mutation is selected from KRAS^{G12A}, KRAS^{G12C}, KRAS^{G12D}, KRAS^{G12R}, KRAS^{G12S} KRAS^{G12V}, KRAS^{G13A}, KRAS^{G13C}, KRAS^{G13D}, KRAS^{G13R}, KRAS^{G12S}, KRAS^{G13V}, KRAS^{Q61E}, KRAS^{Q61H}, KRAS^{Q61K}, KRAS^{Q61L}, KRAS^{Q61P}, KRAS^{Q61R}, KRAS^{A146P}, KRAS^{A146T}, and KRAS^{A146V}.

70. (canceled)

71. The method of claim 60, wherein the disease or disorder is cancer.

72. The method of claim 71, wherein the cancer is selected from pancreatic adenocarcinoma, colorectal adenocarcinoma, lung adenocarcinoma, prostate adenocarcinoma, uterine endometrial carcinoma, cholangiocarcinoma, testicular cancer, cervical squamous cell carcinoma, appendiceal cancer, myelodysplastic syndrome, acute myeloid leukemia (AML), and juvenile myelomonocytic leukemia.

73. The method of claim 67, wherein the KRAS mutation is selected from KRAS^{V14I}, KRAS^{Q22R}, KRAS^{P34L}, KRAS^{P34Q}, KRAS^{P34R}, KRAS^{I36M}, KRAS^{T58I}, KRAS^{G60R}, KRAS^{G60S}, KRAS^{G60V}, KRAS^{Y71H}, KRAS^{K147E}, KRAS^{D153V}, and KRAS^{V152G}.

74. The method of claim 60, wherein the disease or disorder is a developmental disease or syndrome.

75. (canceled)
